(19) United States
(12) Patent Application Publication
(10) Pub. No.: US 2013/0338367 A1
(43) Pub. Date: Dec. 19, 2013

(54) PYRAZOLE DERIVATIVES AND PESTICIDES

(71) Applicants: Akira Numata, Funabashi-shi (JP); Daisuke Tanima, Funabashi-shi (JP); Masanori Ando, Minamisaitama-gun (JP); Fumiyo Saito, Funabashi-shi (JP); Yuji Iwawaki, Funabashi-shi (JP)

(72) Inventors: Akira Numata, Funabashi-shi (JP); Daisuke Tanima, Funabashi-shi (JP); Masanori Ando, Minamisaitama-gun (JP); Fumiyo Saito, Funabashi-shi (JP); Yuji Iwawaki, Funabashi-shi (JP)

(73) Assignee: Nissan Chemical Industries, Ltd., Tokyo (JP)

(21) Appl. No.: 13/984,796

(22) PCT Filed: Feb. 9, 2012
(86) PCT No.: PCT/JP12/52999

§ 371 Date: Aug. 9, 2013

(30) Foreign Application Priority Data
Feb. 9, 2011 (JP) 2011-025875
May 12, 2011 (JP) 2011-106993
Jun. 29, 2011 (JP) 2011-143871
Aug. 11, 2011 (JP) 2011-176256
Oct. 11, 2011 (JP) 2011-223837
Oct. 26, 2011 (JP) 2011-234671
Nov. 18, 2011 (JP) 2011-252596

(51) Int. Cl. C07D 401/04; A01N 43/56; C07D 401/14

(52) U.S. Cl. CPC C07D 401/04; C07D 401/14; A01N 43/56
USPC 546/193; 546/275.4; 546/256

(57) ABSTRACT

Novel pesticide, especially insecticides or miticides are provided.

Pyrazole derivative and pesticides represented by the formula (1):

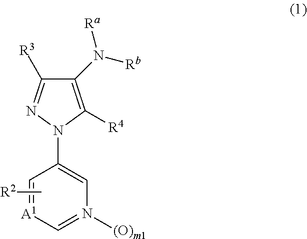

wherein A¹ is —N(O)ₘ₂ or —CR¹, each of R¹, R³, R⁴ is a hydrogen atom, a halogen atom, C₁-C₆ alkyl or the like, R^{a} is a hydrogen atom, C₁-C₆ alkyl or the like, R^{b} is —C(O)R⁷, —C(O)N(R^{8a})R⁸ or the like, each of R⁷, R⁸, R^{8a} is a hydrogen atom, C₁-C₆ alkyl or the like, and m1 is an integer of 0 or 1.

### TECHNICAL FIELD

The present invention relates to a pesticide containing pyrazole derivatives and salts thereof and pesticides containing them as an active ingredient. In the present invention, a pesticide means an agent for controlling agriculturally, zootechnically or hygienically noxious arthropods (such as internal and external parasites in and on farm animals or pet mammals and birds and hygienic or annoying home and workplace insects) as target pests. In the present invention, an agrochemical means an insecticide, miticide, nematocide, herbicide or fungicide or the like used in the field of agriculture.

### BACKGROUND ART

Although pyrazole derivatives are disclosed, for example, in Patent Documents 1 to 9, they do not disclose the pyrazole derivatives of the present invention at all. Their usefulness as pesticides, especially, as insecticides, miticides or parasticides for internal or external, mammal or bird parasites is not known at all, either.

### PRIOR ART DOCUMENT

Patent Document 1: WO2011/128304

Patent Document 2: WO2009/076454

Patent Document 3: WO2008/044767

Patent Document 4: US-A-2004-214838

Patent Document 5: JP-A-2003-313103

Patent Document 6: WO99/010350

Patent Document 7: WO97/034893

Patent Document 8: JP-A-63-174905

Patent Document 9: JP-A-62-153273

### DISCLOSURE OF THE INVENTION

### Technical Problem

With the advance of development of pesticides targeted at various pest insects such as agricultural pest insects, forest pest insects or hygienic pest insects, various pesticides have been put into practical use.

However, recently, control of pest insects with conventional insecticides or fungicides has become difficult in more and more cases, as pest insects acquire resistance to them over many years of their use. Problems of the high toxicity of some conventional pesticides and of the disturbance of the ecosystem by some conventional pesticides which remain in the environment for a long period are becoming apparent. Under these circumstances, development of novel pesticides with high pesticidal activity, low toxicity and low persistence is always expected.

### Solution to Problems

As a result of their extensive studies to accomplish the above object and, the present inventors found that the novel pyrazole derivative of the present invention represented by the following formula (I) are very useful compounds which are excellent in pesticidal activities, especially in insecticidal and miticidal activities, and have little harmful effect on non-target organisms such as mammals, fish and beneficial insects and accomplished the present invention.

Namely, the present invention relates to the following [1] to [25].

[1] A pyrazole derivative represented by the formula (1):

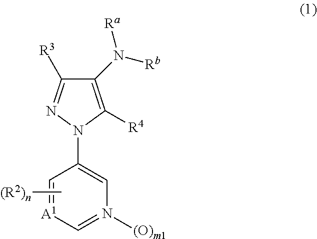

wherein A¹ is —N(—O)ₘ₂ or —CR¹,
each of R¹ and R³ is independently a hydrogen atom, a halogen atom, cyano, nitro, —OH, —SH, —NH₂, C₁-C₆ alkyl, (C₁-C₆) alkyl optionally substituted with R^{28a}, C₃-C₈ cycloalkyl, (C₃-C₈) cycloalkyl optionally substituted with R^{28a}, C₂-C₆ alkenyl, (C₂-C₆) alkenyl optionally substituted with R^{28a}, C₃-C₈ cycloalkenyl, (C₃-C₈) cycloalkenyl optionally substituted with R^{28a}, C₂-C₆ alkynyl, (C₂-C₆) alkynyl optionally substituted with R^{28a}, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylcarbonyl, C₃-C₈ cycloalkylcarbonyl, C₁-C₆ haloalkylcarbonyl, C₃-C₈ halocycloalkylcarbonyl, C₁-C₆ alkoxycarbonyl, C₁-C₆ haloalkoxycarbonyl, C₁-C₆ alkylaminosulfonyl, di(C₁-C₆ alkyl)aminosulfonyl, C₁-C₆ alkylamino or di(C₁-C₆ alkyl)amino,
R² is a halogen atom, cyano, nitro, —OH, —SH, —NH₂, C₁-C₆ alkyl, (C₁-C₆) alkyl optionally substituted with R^{28a}, C₃-C₈ cycloalkyl, (C₃-C₈) cycloalkyl optionally substituted with R^{28a}, C₂-C₆ alkenyl, (C₂-C₆) alkenyl optionally substituted with R^{28a}, C₃-C₈ cycloalkenyl, (C₃-C₈) cycloalkenyl optionally substituted with R^{28a}, C₂-C₆ alkynyl, (C₂-C₆) alkynyl optionally substituted with R^{28a}, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylcarbonyl, C₃-C₈ cycloalkylcarbonyl, C₁-C₆ haloalkylcarbonyl, C₃-C₈ halocycloalkylcarbonyl, C₁-C₆ alkoxycarbonyl, C₁-C₆ haloalkoxycarbonyl, C₁-C₆ alkylaminosulfonyl, di(C₁-C₆ alkyl)aminosulfonyl, C₁-C₆ alkylamino or di(C₁-C₆ alkyl)amino, provided that when n is an integer of at least 2, each R² may be identical with or different from one another,
R⁴ is a hydrogen atom, a halogen atom, cyano, nitro, —OH, —SH, —NH₂, C₁-C₆ alkyl, (C₁-C₆) alkyl optionally substituted with R^{28a}, C₃-C₈ cycloalkyl, (C₃-C₈) cycloalkyl optionally substituted with R^{28a}, C₂-C₆ alkenyl, (C₂-C₆) alkenyl optionally substituted with R^{28a}, C₃-C₈ cycloalkenyl, (C₃-C₈) cycloalkenyl optionally substituted with R^{28a}, C₂-C₆ alkynyl, (C₂-C₆) alkynyl optionally substituted with R^{28a}, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylcarbonyl, C₃-C₈ cycloalkylcarbonyl, C₁-C₆ haloalkylcarbonyl, C₃-C₈ halocycloalkylcarbonyl, C₁-C₆ alkylamino or di(C₁-C₆ alkyl)amino,
R^{a} is a hydrogen atom, cyano, C₁-C₁₅ alkyl, (C₁-C₁₅) alkyl optionally substituted with R⁵, C₃-C₁₅ cycloalkyl, (C₃-C₁₅) cycloalkyl optionally substituted with R⁵, C₂-C₁₅ alkenyl, (C₂-C₁₅) alkenyl optionally substituted with R⁵, C₃-C₁₂ cycloalkenyl, (C₃-C₁₅) cycloalkenyl optionally substituted with R⁵, C₂-C₁₂ alkynyl, (C₂-C₁₅) alkynyl optionally substituted with R⁵, —OR⁶, —S(O)ᵣR⁶, —C(O)R^{7a}, —C(O)OR^{6a}, —NR^{8c}R^{8d}, —C(═NR^{8b})R^{7a}, —S(O)ᵣN(R^{8a})R⁸, phenyl, phenyl substituted with (Z)_{q}, naphthyl, naphthyl substituted with (Z)_{q} or D1-1 to D1-99,
R^{b} is —S(O)ᵣR⁶, —C(O)R⁷, —C(S)R⁷, —C(O)OR^{6a}, —C(O)SR^{6a}, —C(S)OR^{6a}, —C(S)SR^{6a}, —C(O)N(R^{8a})R⁸, —C(S)N(R^{8a})R⁸, —C(O)N(R^{8b})N(R^{8a})R⁸, —C(O)N(R^{8a})OR^{6a}, —C(═NR^{8b})OR^{6a}, —C(═NR^{8b})SR^{6a}, —C(═NR^{8b})N(R^{8a})R⁸, —C(═NR^{8b})R⁷, D1-49, D1-51, D1-53, D1-59, D1-61 or D1-63 or R^{b} may form ═C(R^{b2})R^{b3} together with R^{a},
R^{b2} is a hydrogen atom, C₁-C₁₅ alkyl or —S(O)ᵣR⁶,
R^{b3} is (C₁-C₁₅) alkyl optionally substituted with R¹⁴, —OR⁶, —S(O)ᵣR⁶ or —N(R^{8b})R⁸, or R^{b3} may form, together with R^{b2}, a C₄-C₆ alkylene chain or a C₄-C₆ alkenylene chain to form a 5 to 7-membered ring together with the carbon atom attached to R^{b3} and R^{b2}, wherein the alkylene chain or the alkenylene chain may contain from 1 to 3 oxygen atoms, sulfur atoms or nitrogen atoms and may optionally be substituted with a halogen atom, a cyano group, a nitro group, C₁-C₆ alkyl, (C₁-C₁₂) alkyl optionally substituted with R¹⁴, —S(O)ᵣR⁶, —C(O)R⁷, —C(S)R⁷, —C(O)OR^{6a}, —C(O)SR^{6a}, —C(S)OR^{6a}, —C(S)SR^{6a}, —C(O)N(R^{8a})R⁸, —C(S)N(R^{8a})R⁸, —C(O)N(R^{8b})N(R^{8a})R⁸, —C(O)N(R^{8a})OR⁶, —C(═NR^{8b})OR^{6a}, —C(═NR^{8b})SR^{6a}, —C(═NR^{8b})N(R^{8a})R⁸, —C(═NR^{8b})R⁷, phenyl, phenyl substituted with (Z)_{q}, an oxo group, a thioxo group, ═NR^{8b} or a C₁-C₆ alkylidene group,
R⁵ is a halogen atom, cyano, nitro, C₃-C₁₅ cycloalkyl, (C₃-C₁₅) cycloalkyl optionally substituted with R¹⁴, —OH, —OR¹¹, —SH, —S(O)ᵣR¹¹, —C(O)R¹², —C(O)OR^{11a}, —C(O)SR^{6a}, —C(S)OR^{6a}, —C(S)SR^{6a}, —C(O)N(R^{13a})R¹³, —C(S)N(R^{13a})R¹³, —C(═NOH)R¹², —C(═NOR¹¹)R¹², —N(R^{13a})R¹³, —Si(R^{9a})(R^{9b})R⁹, —P(O)(OR¹⁰)₂, —P(S)(OR¹⁰)₂, phenyl, phenyl substituted with (Z³)_{q}, naphthyl, naphthyl substituted with (Z)_{q} or D1-1 to D1-99,
each of R⁶ and R^{6a} is independently C₁-C₁₅ alkyl, (C₁-C₁₅) alkyl optionally substituted with R¹⁴, C₃-C₁₅ cycloalkyl, (C₃-C₁₅) cycloalkyl optionally substituted with R¹⁴, C₂-C₁₅ alkenyl, (C₂-C₁₅) alkenyl optionally substituted with R¹⁴, C₃-C₁₅ cycloalkenyl, (C₃-C₁₅) cycloalkenyl optionally substituted with R¹⁴, C₂-C₁₅ alkynyl, (C₂-C₁₅) alkynyl optionally substituted with R¹⁴, phenyl, phenyl substituted with (Z)_{q}, naphthyl, naphthyl substituted with (Z)_{q} or D1-1 to D1-99,
R⁷ is a hydrogen atom, C₁-C₁₅ alkyl, (C₁-C₁₅) alkyl optionally substituted with R¹⁴, C₃-C₁₅ cycloalkyl, (C₃-C₁₅) cycloalkyl optionally substituted with R¹⁴, C₂-C₁₅ alkenyl, (C₂-C₁₅) alkenyl optionally substituted with R¹⁴, C₃-C₁₅ cycloalkenyl, (C₃-C₁₅) cycloalkenyl optionally substituted with R¹⁴, C₂-C₁₅ alkynyl, (C₂-C₁₅) alkynyl optionally substituted with R¹⁴, —C(O)R¹², —C(O)OR¹¹, —C(O)N(R^{13a})R¹³, —C(S)N(R^{13a})R¹³, —C(═NOH)R¹², —C(═NOR¹¹)R¹², —C{═NN(R^{13a})R¹³}R¹², phenyl, phenyl substituted with (Z)_{q}, naphthyl, naphthyl substituted with (Z)_{q} or D1-1 to D1-99, or R⁷ may form, together with R^{a}, a C₂-C₆ alkylene chain or a C₂-C₆ alkenylene chain containing a double bond to form a 4 to 8-membered ring together with the carbon atom attached to R⁷ and the nitrogen atom attached to R^{a}, wherein the alkylene chain or the alkenylene chain may contain one or two oxygen atoms, sulfur atoms or nitrogen atoms and may optionally be substituted with a halogen atom, cyano, nitro, C₁-C₆ alkyl, (C₁-C₆) alkyl optionally substituted with R¹⁴, —OH, —OR¹¹, —SH, —S(O)ᵣR¹¹, an oxo group, a thioxo group, ═NR^{8b} or a C₁-C₆ alkylidene group,
R^{7a} is a hydrogen atom or R^{6a},
R⁸ is a hydrogen atom, cyano, R^{6a}, —S(O)ᵣR¹¹, —C(O)R¹², —C(O)OR^{11a}, —C(O)N(R^{13a})R¹³, —C(S)N(R^{13a})R¹³, —C(═NOH)R¹², —C(═NOR¹¹)R¹², —S(O)₂N(R^{13a})R¹³, phenyl, phenyl substituted with (Z)_{q}, naphthyl, naphthyl substituted with (Z)_{q} or D1-1 to D1-99, or R⁸ may form, together with R^{8a}, a C₂-C₇ alkylene chain to form a 3 to 8-membered ring together with the nitrogen atom attached to R⁸ and R^{8a}, wherein the alkylene chain may contain an oxygen atom, a sulfur atom or a nitrogen atom and may optionally be substituted with a halogen atom, cyano, C₁-C₆ alkyl, (C₁-C₆) alkyl optionally substituted with R¹⁴, phenyl, phenyl substituted with (Z)_{q}, an oxo group or a thioxo group, or R⁸ may form ═C(R^{8f})R^{8e} together with R^{8a},
R^{8a} is a hydrogen atom, cyano, C₁-C₁₅ alkyl, (C₁-C₁₅) alkyl optionally substituted with R^{14a}, C₃-C₁₅ cycloalkyl, (C₃-C₁₅) cycloalkyl optionally substituted with R^{14a}, C₂-C₁₅ alkenyl, (C₂-C₁₅) alke or nyl optionally substituted with R^{14a}, C₃-C₁₅ cycloalkenyl, (C₃-C₁₅) cycloalkenyl optionally substituted with R^{14a}, C₂-C₁₅ alkynyl or (C₂-C₁₅) alkynyl optionally substituted with R^{14a}, or R^{8a} may form, together with R^{a}, a C₂-C₅ alkylene chain to form a 5 to 8-membered ring together with the nitrogen atom attached to R^{8a} and the nitrogen atom attached to R^{a}, wherein the alkylene chain may contain an oxygen atom, a sulfur atom or a nitrogen atom and may optionally be substituted with a halogen atom, cyano, nitro, C₁-C₆ alkyl, (C₁-C₆) alkyl optionally substituted with R¹⁴, —OH, —OR¹¹, —SH, —S(O)ᵣR¹¹, an oxo group, a thioxo group or ═NR^{8b},
R^{8b} is a hydrogen atom, cyano, nitro, C₁-C₁₅ alkyl, (C₁-C₁₅) alkyl optionally substituted with R¹⁴, C₃-C₁₅ cycloalkyl, (C₃-C₁₅) cycloalkyl optionally substituted with R¹⁴, C₂-C₁₅ alkenyl, (C₂-C₁₅) alkenyl optionally substituted with R¹⁴, C₃-C₁₅ cycloalkenyl, (C₃-C₁₅) cycloalkenyl optionally substituted with R¹⁴, C₂-C₁₅ alkynyl, (C₂-C₁₅) alkynyl optionally substituted with R¹⁴, —OR¹¹, —S(O)ᵣR¹¹, —C(O)OR^{11a}, —C(O)R¹², —C(O)N(R^{13a})R¹³, —C(S)N(R^{13a})R¹³ or —S(O)₂N(R^{13a})R¹³,
each of R^{8c} and R^{8d} is independently a hydrogen atom, C₁-C₆ alkyl, —S(O)ᵣR¹¹, —C(O)OR^{11a}, —C(O)R¹², —C(O)N(R^{13a})R¹³ or —C(S)N(R^{13a})R¹³,
R^{8e} is a hydrogen atom, C₁-C₁₅ alkyl or —S(O)ᵣR¹¹,
R^{8f} is C₁-C₁₅ alkyl, (C₁-C₁₅) alkyl optionally substituted with R¹⁴, —OR¹¹, —S(O)ᵣR¹¹, —N(R^{13a})R¹³, phenyl, phenyl substituted with (Z)_{q}, naphthyl, naphthyl substituted with (Z)_{q} or D1-1 to D1-99,
each of R⁹, R^{9a} and R^{9b} is independently C₁-C₆ alkyl,
R¹⁰ is a hydrogen atom or C₁-C₆ alkyl,
each of R¹¹ and R^{11a} is independently C₁-C₆ alkyl, (C₁-C₆) alkyl optionally substituted with R^{14a}, C₂-C₆ alkenyl, (C₂-C₆) alkenyl optionally substituted with R^{14a}, C₃-C₈ cycloalkenyl, (C₃-C₈) cycloalkenyl optionally substituted with R^{14a}, C₂-C₆ alkynyl, (C₂-C₆) alkynyl optionally substituted with R^{14a}, C₃-C₈ cycloalkyl, (C₃-C₈) cycloalkyl optionally substituted with R^{14a}, —Si(R^{9a})(R^{9b})R⁹, phenyl, phenyl substituted with (Z)_{q}, naphthyl, naphthyl substituted with (Z)_{q} or D1-1 to D1-99,
R¹² is a hydrogen atom, R^{11a}, —C(O)R¹⁶, —C(═NOH)R¹⁶, —C(═NOR¹⁵)R¹⁶, phenyl, phenyl substituted with (Z)_{q}, naphthyl, naphthyl substituted with (Z)_{q} or or D1-1 to D1-99,
each of R¹³ and R^{13a} is independently a hydrogen atom, R^{11a}, —S(O)ᵣR¹⁵, —C(O)OR^{15a}, —C(O)R¹⁶, —C(O)N(R^{17a})R¹⁷, —C(S)N(R^{17a})R¹⁷, —S(O)₂N(R^{17a})R¹⁷, phenyl, phenyl substituted with (Z)_{q}, naphthyl, naphthyl substituted with (Z)_{q} or or D1-1 to D1-99, or R¹³ and R^{13a} may form, together with each other, a C₂-C₇ alkylene chain to form a 3 to 8-membered ring together with the nitrogen atom attached to R¹³ and R^{13a}, wherein the alkylene chain may contain an oxygen atom, a sulfur atom or a nitrogen atom and may optionally be substituted with a halogen atom, cyano, C₁-C₆ alkyl, (C₁-C₆) alkyl optionally substituted with R¹⁴, phenyl, phenyl substituted with (Z)_{q}, an oxo group or a thioxo group,
each of R¹⁴ and R^{14a} is independently a halogen atom, cyano, nitro, C₃-C₈ cycloalkyl, (C₃-C₈) cycloalkyl optionally substituted with R¹⁹, —OH, —OR¹⁵, —SH, —S(O)ᵣR¹⁵, —S(═NR^{17b})R^{15a}, —S(O)(═NR^{17b})R^{15a}, —C(O)OH, —C(O)OR^{15a}, —C(O)SR^{15a}, —C(S)OR^{15a}, —C(S)SR^{15a}, —C(O)R¹⁶, —C(O)N(R^{17a})R¹⁷, —C(S)N(R^{17a})R¹⁷, —N(R^{17a})R¹⁷, —C(═NOH)R¹⁶, —C(═NOR¹⁵)R¹⁶, —ON═C(R^{16a})R¹⁶, —S(O)₂N(R^{17a})R¹⁷, —Si(R^{9a})(R^{9b})R⁹, —P(O)(OR¹⁰)₂, —P(S)(OR¹⁰)₂, phenyl, phenyl substituted with (Z)_{q}, naphthyl, naphthyl substituted with (Z)_{q} or D1-1 to D1-99,
each of R¹⁵ and R^{15a} is independently cyano, C₁-C₆ alkyl, (C₁-C₆) alkyl optionally substituted with R¹⁹, C₂-C₆ alkenyl, (C₂-C₆) alkenyl optionally substituted with R¹⁹, C₂-C₆ alkynyl, (C₂-C₆) alkynyl optionally substituted with R¹⁹, C₃-C₈ cycloalkyl, (C₃-C₈) cycloalkyl optionally substituted with R¹⁹, C₃-C₈ cycloalkenyl, (C₃-C₈) cycloalkenyl optionally substituted with R¹⁹, —S(O)ᵣR²⁰, —C(O)OR^{20a}, —C(O)R²¹, —C(O)N(R^{22a})R²², —C(S)N(R^{22a})R²², phenyl, phenyl substituted with (Z)_{q2}, naphthyl, naphthyl substituted with (Z)_{q} or D1-1 to D1-99,
each of R¹⁶ and R^{16a} is independently a hydrogen atom, C₁-C₆ alkyl, (C₁-C₆) alkyl optionally substituted with R¹⁹, C₂-C₆ alkenyl, (C₂-C₆) alkenyl optionally substituted with R¹⁹, C₂-C₆ alkynyl, (C₂-C₆) alkynyl optionally substituted with R¹⁹, C₃-C₈ cycloalkyl, (C₃-C₈) cycloalkyl optionally substituted with R¹⁹, C₃-C₈ cycloalkenyl, (C₃-C₈) cycloalkenyl optionally substituted with R¹⁹, —C(O)R²¹, phenyl, phenyl substituted with (Z)_{q}, naphthyl, naphthyl substituted with (Z)_{q} or D1-1 to D1-99,
each of R¹⁷ and R^{17a} is independently a hydrogen atom, cyano, C₁-C₆ alkyl, (C₁-C₆) alkyl optionally substituted with R¹⁹, C₂-C₆ alkenyl, (C₂-C₆) alkenyl optionally substituted with R¹⁹, C₂-C₆ alkynyl, (C₂-C₆) alkynyl optionally substituted with R¹⁹, C₃-C₈ cycloalkyl, (C₃-C₈) cycloalkyl optionally substituted with R¹⁹, C₃-C₈ cycloalkenyl, (C₃-C₈) cycloalkenyl optionally substituted with R¹⁹, —CHO, —S(O)ᵣR²⁰, —C(O)R²¹, —C(O)OR^{20a}, —C(O)N(R^{22a})R²², —C(S)N(R^{22a})R²² or —S(O)₂N(R^{22a})R²², or R¹⁷ and R^{17a} may form, together with each other, a C₂-C₇ alkylene chain to form a 3 to 8-membered ring together with the nitrogen atom attached to R¹⁷ and R^{17a}, wherein the alkylene chain may contain an oxygen atom, a sulfur atom or a nitrogen atom and may optionally be substituted with a halogen atom, cyano, C₁-C₆ alkyl, (C₁-C₆) alkyl optionally substituted with R¹⁴, phenyl, phenyl substituted with (Z)_{q}, an oxo group or a thioxo group,
R^{17b} is a hydrogen atom, cyano, nitro, —OR²⁰, —S(O)ᵣR²⁰, —C(O)OR^{20a}, —C(O)R²¹, —C(O)N(R^{22a})R²², —C(S)N(R^{22a})R²² or —S(O)₂N(R^{22a})R²²,
R¹⁹ is a halogen atom, cyano, C₃-C₈ cycloalkyl, —OR²⁰, —S(O)ᵣR²⁰, —C(O)OR^{20a}, —C(O)N(R^{22a})R²², —C(S)N(R^{22a})R²², —C(═NOR²⁰)R²¹, —Si(R^{9a})(R^{9b})R⁹, phenyl, phenyl substituted with (Z)_{q}, naphthyl, naphthyl substituted with (Z)_{q} or D1-1 to D1-99,
each of R²⁰ and R²¹ is independently a hydrogen atom, C₁-C₆ alkyl, (C₁-C₆) alkyl optionally substituted with R³², C₂-C₆ alkenyl, (C₂-C₆) alkenyl optionally substituted with R³², C₂-C₆ alkynyl, (C₂-C₆) alkynyl optionally substituted with R³², C₃-C₈ cycloalkyl, (C₃-C₈) cycloalkyl optionally substituted with R³², C₃-C₈ cycloalkenyl, (C₃-C₈) cycloalkenyl optionally substituted with R³², phenyl, phenyl substituted with (Z)_{q}, naphthyl, naphthyl substituted with (Z)_{q} or D1-1 to D1-99,
each of R^{20a}, R²² and R^{22a} is independently a hydrogen atom, C₁-C₆ alkyl, (C₁-C₆) alkyl optionally substituted with R³², C₂-C₆ alkenyl, (C₂-C₆) alkenyl optionally substituted with R³², C₂-C₆ alkynyl, (C₂-C₆) alkynyl optionally substituted with R³², C₃-C₈ cycloalkyl, (C₃-C₈) cycloalkyl optionally substituted with R³², C₃-C₈ cycloalkenyl, (C₃-C₈) cycloalkenyl optionally substituted with R³², phenyl, phenyl substituted with (Z)_{q}, naphthyl, naphthyl substituted with (Z)_{q} or D1-1 to D1-99,
D1-1 to D1-99 are rings represented by the following structures, respectively,

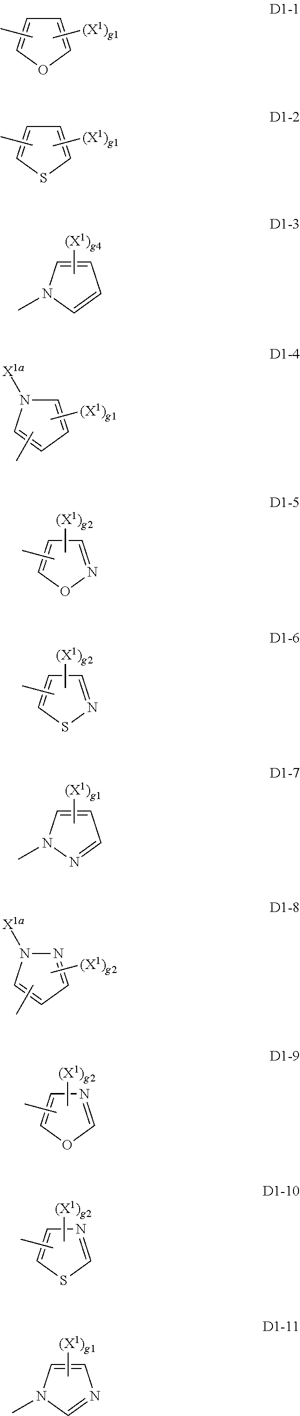

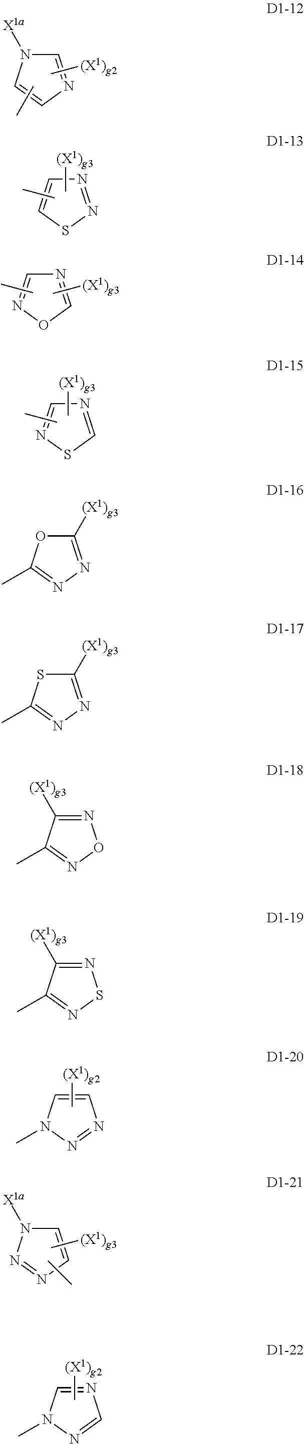

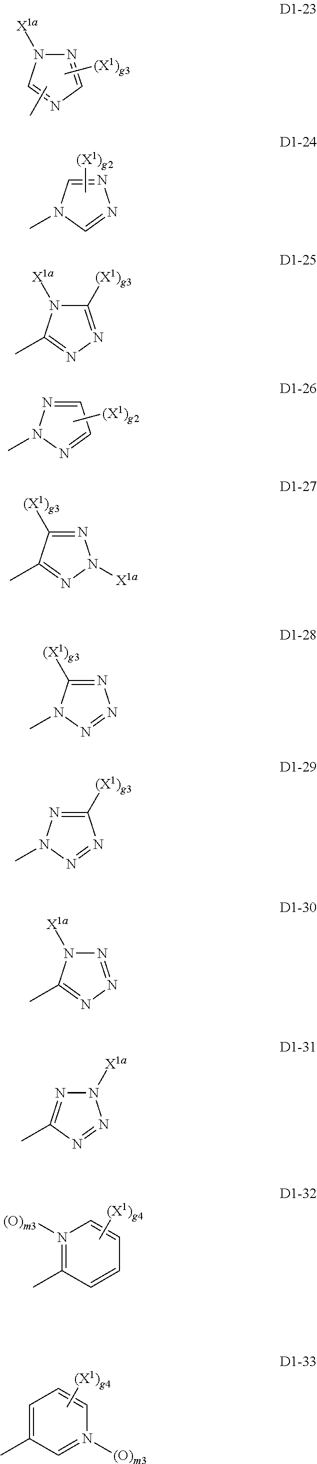

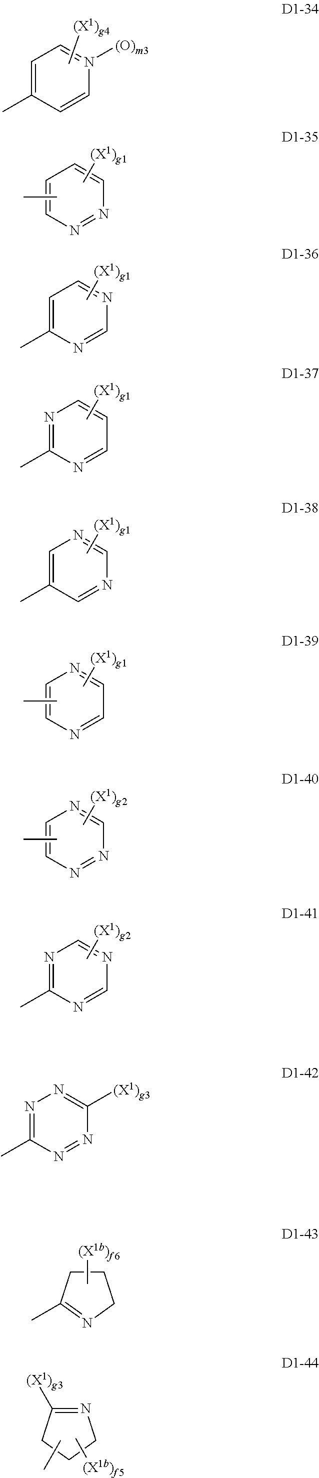

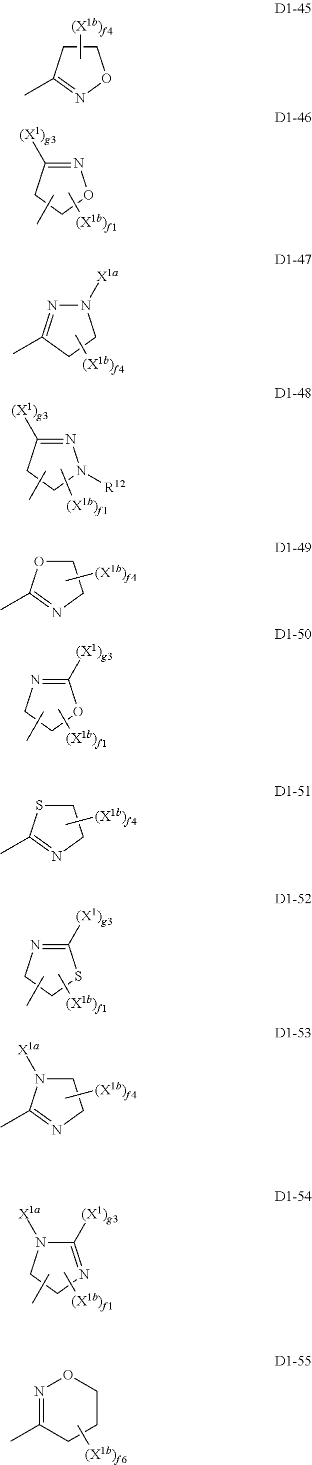

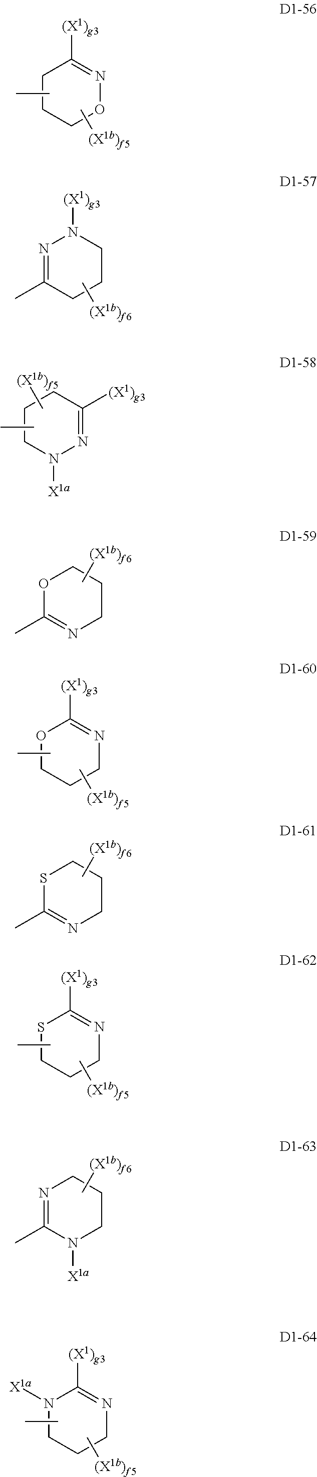

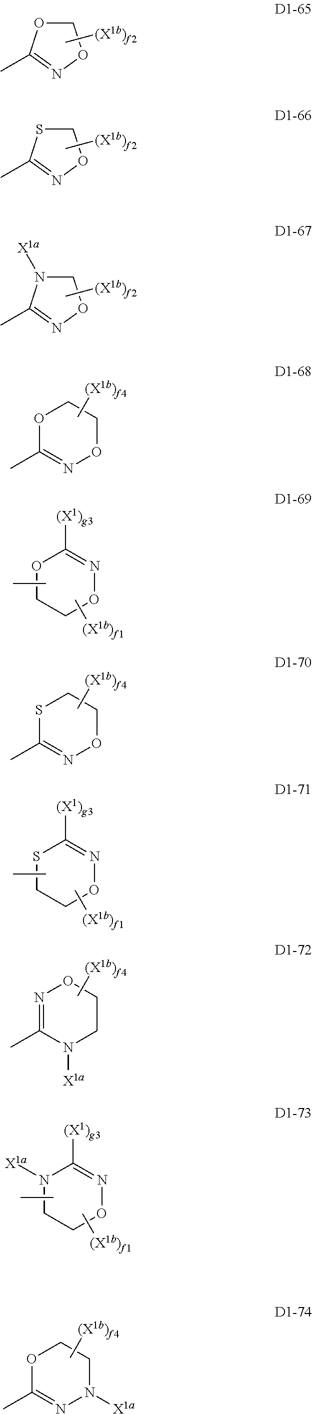

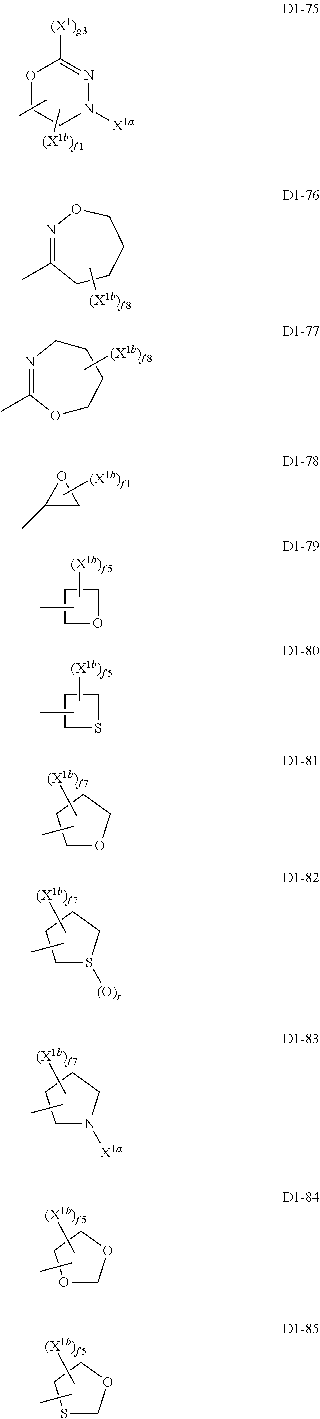

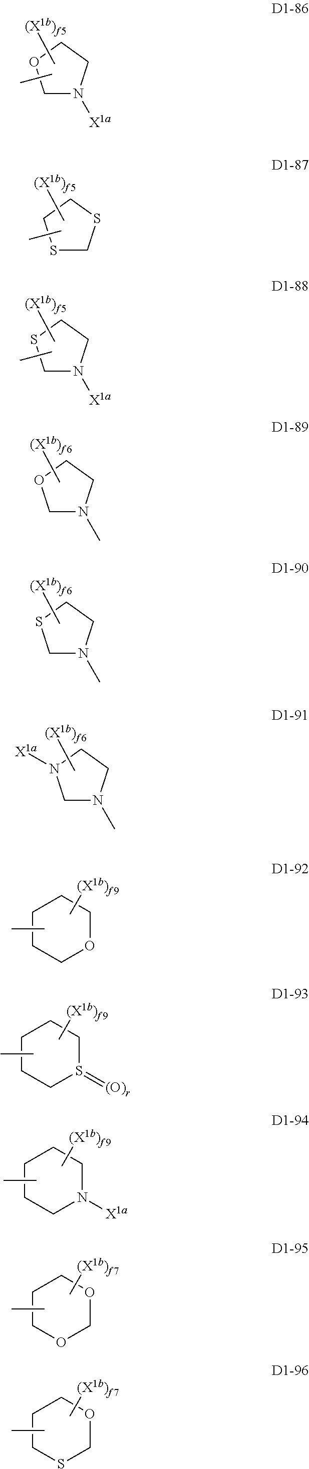

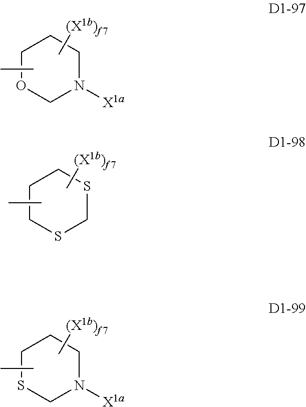

X¹ is a halogen atom, cyano, nitro, —OH, —SH or R²⁴, provided that when g1, g2 or g4 is an integer of at least 2, each X¹ may be identical with or different from one another, and when there are two neighboring X¹'s, the two neighboring X¹'s may form —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —CH₂CH₂CH₂S— or —OCH₂CH₂S— to form, together with the carbon atoms attached to the X¹'s, a 5-membered ring or 6-membered ring which may have one or more hydrogen atoms on the ring-constituting carbon atoms optionally replaced by one or more halogen atoms, one or more C₁-C₆ alkyl groups, one or more C₁-C₆ haloalkyl groups, one or more C₁-C₆ alkoxy groups, one or more C₁-C₆ alkylthio groups, one or more C₁-C₆ alkylsulfinyl groups or one or more C₁-C₆ alkylsulfonyl groups,
X^{1a} is a hydrogen atom, cyano, —OH or R²⁴,
X^{1b} is a halogen atom, cyano, nitro, —OH, —SH or R²⁴, provided that when f1, f2, f4, f5, f6, f7, f8 or f9 is an integer of at least 2, each X^{1b} may be identical with or different from one another, and when there are two X^{1b}'s on the same carbon, the two X^{1b}'s may form oxo, thioxo, imino, C₁-C₆ alkylimino, C₁-C₆ alkoxyimino or C₁-C₆ alkylidene together with each other,
Z is a halogen atom, cyano, nitro, —OH, —SH or R²⁴, provided that q is an integer of at least 2, each Z may be identical with or different from one another, and when there are two neighboring Z's, the two neighboring Z's may form —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —CH₂CH₂CH₂S— or —OCH₂CH₂S— to form, together with the carbon atoms attached to the Z's, a 5-membered ring or a 6-membered ring which may have one or more hydrogen atoms on the ring-constituting carbon atoms optionally replaced by one or more halogen atoms, one or more C₁-C₆ alkyl groups, one or more C₁-C₆ haloalkyl groups, one or more C₁-C₆ alkoxy groups, one or more C₁-C₆ alkylthio groups, one or more C₁-C₆ alkylsulfinyl groups or one or more C₁-C₆ alkylsulfonyl groups,
Z³ is a halogen atom, nitro, —OH, —SH or R²⁴, provided that q is an integer of at least 2, each Z³ may be identical with or different from one another, and when there are two neighboring Z³'s, the neighboring two Z's may form —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —CH₂CH₂CH₂S— or —OCH₂CH₂S— to form, together with the carbon atoms attached to the Z³'s, a 5-membered ring or a 6-membered ring which may have one or more hydrogen atoms on the ring-constituting carbon atoms optionally replaced by one or more halogen atoms, one or more C₁-C₆ alkyl groups, one or more C₁-C₆ haloalkyl groups, one or more C₁-C₆ alkoxy groups, one or more C₁-C₆ alkylthio groups, one or more C₁-C₆ alkylsulfinyl groups or one or more C₁-C₆ alkylsulfonyl groups,
R²⁴ is a C₁-C₆ alkyl, (C₁-C₆) alkyl optionally substituted with R²⁸, C₂-C₆ alkenyl, (C₂-C₆) alkenyl optionally substituted with R²⁸, C₃-C₈ cycloalkenyl, (C₃-C₈) cycloalkenyl optionally substituted with R²⁸, C₂-C₆ alkynyl, (C₂-C₆) alkynyl optionally substituted with R²⁸, C₃-C₈ cycloalkyl, (C₃-C₈) cycloalkyl optionally substituted with R²⁸, —OR²⁵, —S(O)ᵣR²⁵, —C(O)OH, —C(O)OR^{25a}, —C(O)R²⁶, —C(O)N(R^{27a})R²⁷, —C(S)N(R^{27a})R²⁷, —N(R^{27a})R²⁷, —C(═NOR²⁵)R²⁶ or —S(O)₂N(R^{27a})R²⁷,
each of R²⁵, R^{25a} and R²⁶ is independently C₁-C₆ alkyl or (C₁-C₆) alkyl optionally substituted with R^{32a},
each of R²⁷ and R^{27a} is independently a hydrogen atom, C₁-C₆ alkyl, —S(O)ᵣR³⁴ or —C(O)R³⁴,
each of R²⁸ and R^{28a} is independently a halogen atom, —OH, —OR²⁹, —SH or —S(O)ᵣR²⁹,
each of R²⁹ and R³³ is independently C₁-C₆ alkyl,
each of R³² and R^{32a} is independently a halogen atom, —OR³³ or —S(O)ᵣR³³,
R³⁴ is C₁-C₆ alkyl, C₃-C₈ cycloalkyl or C₁-C₆ haloalkyl,
each of n, g1 and f1 is independently an integer of from 0 to 3,
each of g2 and f2 is independently an integer of from 0 to 2,
g3 is an integer of from 0 to 1,
each of g4 and f4 is independently an integer of from 0 to 4,
f5 is an integer of from 0 to 5,
f6 is an integer of from 0 to 6,
f7 is an integer of from 0 to 7,
f8 is an integer of from 0 to 8,
f9 is an integer of from 0 to 9,
q is independently an integer of from 1 to 5,
q2 is an integer of from 0, 1 or 3 to 5,
each of m1, m2 and m3 is independently an integer of from 0 or 1, and
r is an integer of from 0 to 2.
[2] The pyrazole derivative or a salt thereof according to [1], wherein R^{b} is —C(O)R⁷, —C(S)R⁷, —C(O)OR^{6a}, —C(O)SR^{6a}, —C(S)OR^{6a}, —C(S)SR^{6a}, —C(O)N(R^{8a})R⁸, —C(S)N(R^{8a})R⁸, —C(O)N(R^{8b})N(R^{8a})R⁸, —C(O)N(R^{8a})OR^{6a}, —C(═NR^{8b})OR^{6a}, —C(═NR^{8b})SR^{6a}, —C(═NR^{8b})N(R^{8a})R⁸, —C(═NR^{8b})R⁷, D1-49, D1-51, D1-53, D1-59, D1-61 or D1-63, or R^{b} may form ═C(R^{b2})R^{b3} together with R^{a}.
[3] The pyrazole derivative or a salt thereof according to [2], wherein A¹ is —CR¹,
each of R¹ and R³ is independently a hydrogen atom, a halogen atom, cyano, —NH₂, C₁-C₆ alkyl, (C₁-C₆) alkyl optionally substituted with R^{28a}, C₁-C₆ alkoxy, C₁-C₆ alkylthio, C₁-C₆ alkylsulfinyl, C₁-C₆ alkylsulfonyl or C₁-C₆ alkoxycarbonyl,
R² is a halogen atom, C₁-C₆ alkyl or C₁-C₆ alkoxy, and
R⁴ is a hydrogen atom, a halogen atom, cyano, —NH₂, C₁-C₆ alkyl, (C₁-C₆) alkyl optionally substituted with R^{28a}, C₁-C₆ alkoxy, C₁-C₆ alkylthio, C₁-C₆ alkylsulfinyl or C₁-C₆ alkylsulfonyl.
[4] The pyrazole derivative or a salt thereof according to [2], wherein A¹ is —N(—O)ₘ₂, R² is a halogen atom, C₁-C₆ alkyl or C₁-C₆ alkoxy, and
each of R³ and R⁴ is independently a hydrogen atom, a halogen atom, C₁-C₆ alkyl or C₁-C₆ alkoxy.
[5] The pyrazole derivative or a salt thereof according to [3], wherein R¹ is a hydrogen atom, a halogen atom, cyano, —NH₂, C₁-C₆ alkyl, C₁-C₆ haloalkyl or C₁-C₆ alkoxy,
R² is C₁-C₆ alkyl,
R³ is a hydrogen atom, a halogen atom, cyano, C₁-C₆ alkyl, (C₁-C₆) alkyl optionally substituted with R^{28a}, C₁-C₆ alkoxy, C₁-C₆ alkylthio, C₁-C₆ alkylsulfonyl or C₁-C₆ alkoxycarbonyl,
R⁴ is a hydrogen atom, a halogen atom, C₁-C₆ alkyl or C₁-C₆ alkylthio,
R^{a} is a hydrogen atom, C₁-C₆ alkyl, (C₁-C₆) alkyl optionally substituted with R⁵, C₂-C₆ alkenyl, C₂-C₆ alkynyl, —S(O)ᵣR⁶, —C(O)R^{7a}, —C(O)OR^{6a}, —NR^{8c}R^{8d} or —C(═NR^{8b})R^{7a},
R^{b} is —C(O)R⁷, —C(S)R⁷, —C(O)OR^{6a}, —C(O)SR^{6a}, —C(O)N(R^{8a})R⁸, —C(S)N(R^{8a})R⁸, —C(O)N(R^{8b})N(R^{8a})R⁸, —C(═NR^{8b})SR^{6a}, —C(═NR^{8b})N(R^{8a})R⁸, —C(═NR^{8b})R⁷ or D1-51, or R^{b} may form ═C(R^{b2})R^{b3} together with R^{a},
R^{b2} is a hydrogen atom, C₁-C₆ alkyl or —S(O)ᵣR⁶,
R^{b3} is (C₁-C₆) alkyl optionally substituted with R¹⁴, —OR⁶ or —N(R^{8b})R⁸, or R^{b3} may form, together with R^{b2}, a C₄ alkylene chain to form a 5-membered ring together with the carbon atom attached to R^{b3} and R^{b2}, wherein the alkylene chain may contain a sulfur atom and a nitrogen atom and may optionally be substituted with —C(O)R⁷,
R⁵ is a halogen atom, cyano, C₃-C₈ cycloalkyl, —OR¹¹, —S(O)ᵣR¹¹, —C(O)OR^{11a}, —C(═NOR¹¹)R¹², —Si(R^{9a})(R^{9b})R⁹, phenyl or D1-37,
R⁶ is C₁-C₆ alkyl or (C₁-C₆) alkyl optionally substituted with R¹⁴
R^{6a} is C₁-C₆ alkyl or phenyl substituted with (Z)_{q},
R⁷ is C₁-C₁₅ alkyl, (C₁-C₆) alkyl optionally substituted with R¹⁴, C₃-C₈ cycloalkyl, (C₃-C₈) cycloalkyl optionally substituted with R¹⁴, C₂-C₆ alkenyl, (C₂-C₆) alkenyl optionally substituted with R¹⁴, C₃-C₈ cycloalkenyl, C₂-C₆ alkynyl, (C₂-C₆) alkynyl optionally substituted with R¹⁴, —C(O)OR^{11a}, —C(O)R¹², —C(O)N(R^{13a})R¹³, —C(═NOR¹¹)R¹², phenyl substituted with (Z)_{q}, D1-1, D1-2, D1-4, D1-5, D1-6, D1-8, D1-9, D1-10, D1-12, D1-13, D1-19, D1-32, D1-33, D1-35, D1-38, D1-45, D1-81, D1-82, D1-87, D1-88, D1-92 or D1-94, or R⁷ may form, together with R^{a}, a C₂-C₄ alkylene chain or a C₂-C₄ alkenylene chain containing a double bond to form a 4 to 6-membered ring together with the carbon atom attached to R⁷ and the nitrogen atom attached to R^{a}, wherein the alkylene chain may contain one or two oxygen atoms or nitrogen atoms and may optionally be substituted with a halogen atom, C₁-C₆ alkyl, (C₁-C₆) alkyl optionally substituted with R¹⁴, —OR¹¹, —S(O)ᵣR¹¹, an oxo group or a methylidene group,
R^{7a} is a hydrogen atom or C₁-C₆ alkyl,
R⁸ is a hydrogen atom, C₁-C₆ alkyl, (C₁-C₆) alkyl optionally substituted with R¹⁴, C₃-C₈ cycloalkyl, C₂-C₆ alkenyl, C₂-C₆ alkynyl, —C(O)R¹², —C(O)OR^{11a}, —C(═NOR¹¹)R¹², —S(O)₂R^{11a}, phenyl, phenyl substituted with (Z)_{q}, D1-32, D1-33, D1-34 or D1-80,
R^{8a} is a hydrogen atom, C₁-C₆ alkyl or (C₁-C₆) alkyl optionally substituted with R^{14a}, or R^{8a} may form, together with R^{a}, a C₂-C₃ alkylene chain to form a 5 to 6-membered ring together with the nitrogen atom attached to R^{8a} and the nitrogen atom attached to R^{a}, wherein the alkylene chain may contain an oxygen atom and may optionally be substituted with (C₁-C₆) alkyl optionally substituted with R¹⁴ or an oxo group, or R⁸ and R^{8a} may form, together with each other, a C₅ alkylene chain to form a 6-membered ring together with the nitrogen atom attached to R⁸ and R^{8a}, wherein the alkylene chain may optionally be substituted with C₁-C₆ alkyl, or R⁸ may form ═C(R^{8f})R^{8e} together with R^{8a}, R^{8b} is a hydrogen atom, cyano, C₁-C₆ alkyl or —OR¹¹,
R^{8c} is a hydrogen atom, C₁-C₆ alkyl or —C(O)OR^{11a},
R^{8d} is a hydrogen atom, —C(O)OR^{11a} or —C(O)R¹²,
R^{8e} is a hydrogen atom,

### R^{8f} is —N(R^{13a})R¹³,

each of R⁹, R^{9a}, R^{9b} and R¹⁰ is independently C₁-C₆ alkyl,
R¹¹ is C₁-C₆ alkyl, (C₁-C₆) alkyl optionally substituted with R^{14a} or C₁-C₆ alkenyl,
R^{11a} is C₁-C₆ alkyl, (C₁-C₆) alkyl optionally substituted with R^{14a} or phenyl,
R¹² is a hydrogen atom, C₁-C₆ alkyl, (C₁-C₆) alkyl optionally substituted with R^{14a}, C₃-C₈ cycloalkyl, —C(═NOR¹⁵)R¹⁶ or D1-2,
R¹³ is C₁-C₆ alkyl or phenyl,
R^{13a} is a hydrogen atom or C₁-C₆ alkyl,
R¹⁴ is a halogen atom, cyano, C₃-C₈ cycloalkyl, —OH, —OR¹⁵, —SH, —S(O)ᵣR¹⁵, —S(═NR^{17b})R^{15a}, —S(O)(═NR^{17b})R^{15a}, —C(O)OH, —C(O)OR^{15a}, —C(O)N(R^{17a})R¹⁷, —C(═NOR¹⁵)R¹⁶, —N(R^{17a})R¹⁷, —ON═C(R^{16a})R¹⁶, —S(O)₂N(R^{17a})R¹⁷, —Si(R^{9a})(R^{9b})R⁹, —P(O)(OR¹⁰)₂, phenyl, phenyl substituted with (Z)_{q}, D1-1, D1-2, D1-5, D1-7, D1-8, D1-28, D1-32, D1-33, D1-34, D1-84, D1-85, D1-87, D1-93 or D1-98,
R^{14a} is a halogen atom, —OR¹⁵, —S(O)ᵣR¹⁵, —Si(R^{9a})(R^{9b})R⁹ or phenyl,
R¹⁵ is cyano, C₁-C₆ alkyl, C₂-C₆ alkenyl, C₂-C₆ alkynyl, (C₁-C₆) alkyl optionally substituted with R¹⁹, —C(O)R²¹, phenyl substituted with (Z)_{q2}, D1-12, D1-32, D1-37 or D1-51,
R^{15a} is C₁-C₆ alkyl,
R¹⁶ is C₁-C₆ alkyl or (C₁-C₆) alkyl optionally substituted with R¹⁹,
R^{16a} is a hydrogen atom,
R¹⁷ is a hydrogen atom, C₁-C₆ alkyl, (C₁-C₆) alkyl optionally substituted with R¹⁹, —S(O)ᵣR²⁰, —C(O)OR^{20a}, —C(O)R²¹, —C(O)N(R^{22a})R²², —C(S)N(R^{22a})R²², —C(═NOR²⁰)R²¹, —S(O)₂N(R^{22a})R²² or phenyl,
R^{17a} is a hydrogen atom or C₁-C₆ alkyl, or R^{17a} may form ═C(R^{17c})R^{17d} together with R¹⁷, R^{17b} is cyano,

### R^{17c} is —N(R^{22a})R²²

R^{17d} is a hydrogen atom,
R¹⁹ is a halogen atom, cyano, C₃-C₈ cycloalkyl, —OR²⁰, —S(O)ᵣR²⁰, —C(O)OR^{20a}, —C(O)N(R^{22a})R²², —C(═NOR²⁰)R²¹, —Si(R^{9a})(R^{9b})R⁹, phenyl or D1-34,
R²⁰ is C₁-C₆ alkyl or (C₁-C₆) alkyl optionally substituted with R³²
R^{20a} is C₁-C₆ alkyl,
R²¹ is a hydrogen atom, C₁-C₆ alkyl or (C₁-C₆) alkyl optionally substituted with R³²,
R²² is C₁-C₆ alkyl,
R^{22a} is a hydrogen atom or C₁-C₆ alkyl,
X¹ is a halogen atom, cyano, nitro, C₁-C₆ alkyl, (C₁-C₆) alkyl optionally substituted with R²⁸, C₃-C₈ cycloalkyl, —OR²⁵, —S(O)ᵣR²⁵, —C(O)R²⁶, —C(═NOR²⁵)R²⁶ or —S(O)₂N(R^{27a})R²⁷, provided that when g2 is an integer of 2, each X¹ may be identical with or different from each other,
X^{1a} is C₁-C₆ alkyl, (C₁-C₆) alkyl optionally substituted with R²⁸, —C(O)OR^{25a} or —C(O)R²⁶,
X^{1b} is (C₁-C₆) alkyl optionally substituted with R²⁸, provided that when f5 is an integer of 3, each X^{1b} may be identical with or different from one another, and when there are two X^{1b}'s on the same carbon, the two X^{1b}'s may form oxo together with each other,
Z is a halogen atom, nitro, C₁-C₆ alkyl, (C₁-C₆) alkyl optionally substituted with R²⁸, —S(O)ᵣR²⁵, —C(O)OH or —C(O)N(R^{27a})R²⁷, provided that when q is an integer of at least 2, and there are two neighboring Z's, the two neighboring Z's may form —OCH₂O— to form, together with the carbon atoms attached to the Z's, a 5-membered ring which may have one or more hydrogen atoms on the ring-constituting carbon atoms optionally replaced by one or more halogen atoms,
each of R²⁵ and R²⁶ is independently C₁-C₆ alkyl or (C₁-C₆) alkyl optionally substituted with R^{32a},
each of R^{25a}, R²⁹, R³³ and R³⁴ is independently C₁-C₆ alkyl,
R²⁷ is a hydrogen atom or —S(O)ᵣR³⁴,
R^{27a} is a hydrogen atom,
each of R²⁸ and R^{28a} is independently a halogen atom, —OH, —OR²⁹ or —S(O)ᵣR²⁹,
R³² is a halogen atom, —OR³³ or —S(O)ᵣR³³,
R^{32a} is a halogen atom or —S(O)ᵣR³³,
n is an integer of from 0 to 1,
each of g1 and g2 is independently an integer of from 0 to 2,
each of g3, f7, f9 and m3 is independently an integer of 0,
g4 is an integer of from 0 to 1,
f5 is an integer of from 0 to 3,
q is an integer of from 1 to 2,
q2 is an integer of 1, and
r is an integer of from 0 to 2.
[6] The pyrazole derivative or a salt thereof according to [3], wherein R¹, R² and R⁴ are hydrogen atoms,
R^{a} is a hydrogen atom or C₁-C₆ alkyl,

### R^{b} is —C(O)R⁷, —C(O)OR^{6a}, —C(O)N(R^{8a})R⁸ or —C(═NR^{8b})N(R^{8a})R⁸,

each of R^{6a}, R¹¹ and R¹⁵ is independently C₁-C₆ alkyl,
R⁷ is C₁-C₆ alkyl, C₁-C₆ alkyl optionally substituted with R¹⁴, C₁-C₆ alkenyl optionally substituted with R¹⁴ or D1-8, or R⁷ may form, together with R^{a}, a C₃-C₄ alkylene chain or a C₃-C₄ alkenylene chain containing a double bond to form a 5 to 6-membered ring together with the carbon atom attached to R⁷ and the nitrogen atom attached to R^{a}, wherein the alkylene chain may contain an oxygen atom and may optionally be substituted with a halogen atom, (C₁-C₆) alkyl optionally substituted with R¹⁴ or —S(O)ᵣR¹¹,
R⁸ is C₁-C₆ alkyl or C₁-C₆ alkyl optionally substituted with R¹⁴
R^{8a} is a hydrogen atom, or R^{8a} may form, together with R^{a}, a C₂ alkylene chain to form a 6-membered ring together with the nitrogen atom attached to R^{8a} and the nitrogen atom attached to R^{a}, wherein the alkylene chain may contain an oxygen atom,
R^{8b} is cyano,
R¹⁴ is a halogen atom, —OH, —S(O)ᵣR¹⁵ or phenyl substituted with (Z)_{q},
each of X^{1a} and Z is independently C₁-C₆ haloalkyl,
g2 is an integer of 0,
q is an integer of 1, and
r is an integer of from 0 to 2.
[7] The pyrazole derivative or a salt thereof according to [1], wherein R¹, R² and R⁴ are hydrogen atoms,
R^{a} is a hydrogen atom or C₁-C₆ alkyl,

### R^{b} is —S(O)ᵣR⁶,

R⁶ is C₁-C₆ alkyl, C₂-C₆ alkenyl or (C₁-C₆) alkyl optionally substituted with R¹⁴,

### R¹⁴ is —S(O)ᵣR¹⁵,

R¹⁵ is C₁-C₆ alkyl, and
r is an integer of from 0 to 2.
[8] The pyrazole derivative or a salt thereof according to [5], wherein R⁷ is C₁-C₁₅ alkyl, (C₁-C₆) alkyl optionally substituted with R¹⁴, C₃-C₈ cycloalkyl, (C₃-C₈) cycloalkyl optionally substituted with R¹⁴, C₂-C₆ alkenyl, (C₂-C₆) alkenyl optionally substituted with R¹⁴, C₃-C₈ cycloalkenyl, C₂-C₆ alkynyl, (C₂-C₆) alkynyl optionally substituted with R¹⁴, —C(O)OR^{11a}, —C(O)R¹², —C(O)N(R^{13a})R¹³, —C(═NOR¹¹)R¹², phenyl substituted with (Z)_{q}, D1-1, D1-2, D1-4, D1-5, D1-6, D1-8, D1-9, D1-10, D1-12, D1-13, D1-19, D1-32, D1-33, D1-35, D1-38, D1-45, D1-81, D1-82, D1-87, D1-88, D1-92 or D1-94,
R^{8a} is a hydrogen atom, C₁-C₆ alkyl or (C₁-C₆) alkyl optionally substituted with R^{14a}, or R⁸ may form, together with R^{8a}, a C₅ alkylene chain to form a 6-membered ring together with the nitrogen atom attached to R⁸ and R^{8a}, wherein the alkylene chain may optionally be substituted with C₁-C₆ alkyl, or R⁸ and R^{8a} may form ═C(R^{8f})R^{8e} together with each other.
[9] The pyrazole derivative or a salt thereof according to [5], wherein R⁷ is C₁-C₆ alkyl, (C₁-C₆) alkyl optionally substituted with R¹⁴, C₃-C₈ cycloalkyl, (C₃-C₈) cycloalkyl optionally substituted with R¹⁴, C₂-C₆ alkenyl, (C₂-C₆) alkenyl optionally substituted with R¹⁴, C₃-C₈ cycloalkenyl, C₂-C₆ alkynyl, (C₂-C₆) alkynyl optionally substituted with R¹⁴, —C(O)R¹², —C(═NOR¹¹)R¹², D1-1, D1-2, D1-5, D1-6, D1-8, D1-10, D1-12, D1-13, D1-19, D1-32, D1-33, D1-35, D1-38, D1-45, D1-81, D1-82, D1-87 or D1-88, and
R^{8a} is a hydrogen atom or C₁-C₆ alkyl.
[10] The pyrazole derivative or a salt thereof according to [5], wherein R⁷ is C₁-C₆ alkyl, (C₁-C₆) alkyl optionally substituted with R¹⁴, C₃-C₈ cycloalkyl, (C₃-C₈) cycloalkyl optionally substituted with R¹⁴, C₂-C₆ alkenyl, (C₂-C₆) alkenyl optionally substituted with R¹⁴, C₃-C₈ cycloalkenyl, —C(O)R¹², —C(═NOR¹¹)R¹², D1-8 or D1-82, and
R^{8a} is a hydrogen atom or C₁-C₆ alkyl.
[11] The pyrazole derivative or a salt thereof according to [5], wherein R⁷ is C₁-C₆ alkyl, (C₁-C₆) alkyl optionally substituted with R¹⁴, C₂-C₆ alkenyl, (C₂-C₆) alkenyl optionally substituted with R¹, —C(O)R¹², —C(═NOR¹¹)R¹², D1-8 or D1-82, and
R^{8a} is a hydrogen atom or C₁-C₆ alkyl.
[12] The pyrazole derivative or a salt thereof according to [5], wherein R⁷ is C₁-C₆ alkyl, C₁-C₆ alkyl optionally substituted with R¹⁴, C₁-C₆ alkenyl optionally substituted with R¹⁴, —C(O)R¹², D1-8 or D1-82, and
R^{8a} is a hydrogen atom or C₁-C₆ alkyl.
[13] The pyrazole derivative or a salt thereof according to [5], wherein R⁷ may form, together with R^{a}, a C₂-C₄ alkylene chain or a C₂-C₄ alkenylene chain containing a double bond to form a 4 to 6-membered ring together with the carbon atom attached to R⁷ and the nitrogen atom attached to R^{a}, wherein the alkylene chain may contain one or two oxygen atoms or nitrogen atoms and may optionally be substituted with a halogen atom, C₁-C₆ alkyl, (C₁-C₆) alkyl optionally substituted with R¹⁴, —OR¹¹, —S(O)ᵣR¹¹, an oxo group or a methylidene group,
R⁸ is a hydrogen atom, C₁-C₆ alkyl, (C₁-C₆) alkyl optionally substituted with R¹⁴, C₃-C₈ cycloalkyl, C₂-C₆ alkenyl, C₂-C₆ alkynyl, —C(O)R¹², —C(O)OR^{11a}, —C(═NOR¹¹)R¹², —S(O)₂R^{11a}, phenyl, phenyl substituted with (Z)_{q}, D1-32, D1-33, D1-34 or D1-80,
R^{8a} may form, together with R^{a}, a C₂-C₃ alkylene chain to form a 5 to 6-membered ring together with the nitrogen atom attached to R^{8a} and the nitrogen atom attached to R^{a}, wherein the alkylene chain may contain an oxygen atom and may optionally be substituted with (C₁-C₆) alkyl optionally substituted with R¹⁴ or an oxo group,
R¹⁴ is a halogen atom or —S(O)ᵣR¹⁵, and
R¹⁵ is C₁-C₆ alkyl.
[14] The pyrazole derivative or a salt thereof according to [5], wherein R⁷ may form, together with R^{a}, a C₂-C₄ alkylene chain or a C₂-C₄ alkenylene chain containing a double bond to form a 4 to 6-membered ring together with the carbon atom attached to R⁷ and the nitrogen atom attached to R^{a}, wherein the alkylene chain may contain one or two oxygen atoms or nitrogen atoms and may optionally be substituted with a halogen atom, C₁-C₆ alkyl, (C₁-C₆) alkyl optionally substituted with R¹⁴, —OR¹¹, —S(O)ᵣR¹¹, an oxo group or a methylidene group,
R⁸ is a hydrogen atom or C₁-C₆ alkyl optionally substituted with R¹⁴,
R^{8a} may form, together with R^{a}, a C₂ alkylene chain to form a 5 to 6-membered ring together with the nitrogen atom attached to R^{8a} and the nitrogen atom attached to R^{a}, wherein the alkylene chain may contain an oxygen atom and may optionally be substituted with (C₁-C₆) alkyl optionally substituted with R¹⁴ or an oxo group,
R¹⁴ is a halogen atom or —S(O)ᵣR¹⁵, and
R¹⁵ is C₁-C₆ alkyl.
[15] The pyrazole derivative or a salt thereof according to [5], wherein R⁷ may form, together with R^{a}, a C₃-C₄alkylene chain or a C₃-C₄ alkenylene chain containing a double bond to form a 5 to 6-membered ring together with the carbon atom attached to R⁷ and the nitrogen atom attached to R^{a}, wherein the alkylene chain may contain an oxygen atom and may optionally be substituted with a halogen atom, C₁-C₆ alkyl, (C₁-C₆) alkyl optionally substituted with R¹⁴, —S(O)ᵣR¹¹, oxo group or a methylidene group,
R⁸ is a hydrogen atom or C₁-C₆ alkyl optionally substituted with R¹⁴,
R^{8a} may form, together with R^{a}, a C₂ alkylene chain to form a 5- to 6-membered ring together with the nitrogen atom attached to R^{8a} and the nitrogen atom attached to R^{a}, wherein the alkylene chain may contain an oxygen atom and may optionally be substituted with (C₁-C₆) alkyl optionally substituted with R¹⁴ or an oxo group,
R¹⁴ is a halogen atom or —S(O)ᵣR¹⁵, and
R¹⁵ is C₁-C₆ alkyl.
[16] The pyrazole derivative or a salt thereof according to [5], wherein R⁷ may form, together with R^{a}, a C₃-C₄ alkylene chain or a C₃-C₄ alkenylene containing a double bond to form a 5- to 6-membered ring together with the carbon atom attached to R⁷ and the nitrogen atom attached to R^{a}, wherein the alkylene chain may contain an oxygen atom and may optionally be substituted with a halogen atom, C₁-C₆ alkyl, (C₁-C₆) alkyl optionally substituted with R¹⁴, —S(O)ᵣR¹¹, oxo group or a methylidene group,
R⁸ is C₁-C₆ alkyl optionally substituted with R¹⁴,
R^{8a} may form, together with R^{a}, a C₂ alkylene chain to form a 6-membered ring together with the nitrogen atom attached to R^{8a} and the nitrogen atom attached to R^{a}, wherein the alkylene chain may contain an oxygen atom, and
R¹⁴ is a halogen atom.
[17] The pyrazole derivative or a salt thereof according to [4] wherein R⁴ is a hydrogen atom,
R³ is a halogen atom,
R^{a} is C₁-C₆ alkyl,

### R^{b} is —C(O)R⁷,

R⁷ is (C₁-C₆) alkyl optionally substituted with R¹⁴,

### R¹⁴ is —S(O)ᵣR¹⁵,

R¹⁵ is C₁-C₆ alkyl, and
m2, n and r are integers of 0.
[18] A pesticide containing one or more pyrazole derivatives or salts thereof selected from the pyrazole derivatives and salts thereof as defined in [1] to [17], as active ingredient(s).
[19] An agrochemical containing one or more pyrazole derivatives or salts thereof selected from the pyrazole derivatives and salts thereof as defined in [1] to [17], as active ingredient(s).
[20] A parasticide for an internal or external parasite in or on a mammal or bird, which contains one or more pyrazole derivatives or salts thereof selected from the pyrazole derivatives and salts thereof as defined in [1] to [17], as active ingredient(s).
[21] An insecticide or miticide containing one or more pyrazole derivatives or salts thereof selected from the pyrazole derivatives and salts thereof as defined in [1] to [17], as active ingredient(s).
[22] A seed treatment agent containing one or more pyrazole derivatives or salts thereof selected from the pyrazole derivatives and salts thereof as defined in [1] to [17], as active ingredient(s).
[23] The seed treatment agent according to [22], which is used to treat seeds by dipping.
[24] A soil treatment agent containing one or more pyrazole derivatives or salts thereof selected from the pyrazole derivatives and salts thereof as defined in [1] to [17], as active ingredient(s).
[25] The soil treatment agent according to [24], which is used to treat soil by irrigation.

### ADVANTAGEOUS EFFECT(S)OF INVENTION

The compounds of the present invention have excellent insecticidal and miticidal activities on many agricultural pest insects, spider mites, internal or external mammal or bird parasites and have sufficient controlling effect on pest insects which have acquired resistance to conventional insecticides. The compounds of the present invention have little harmful effect on mammals, fish and beneficial insects, show low persistence and are environmentally friendly.

Thus, the present invention can provide useful novel pesticides.

### DESCRIPTION OF EMBODIMENT(S)

The compounds of the present invention can have geometrical isomers such as E-isomers and Z-isomers, depending on the types of substituents in them, and the present invention covers both E-isomers and Z-isomers and mixtures containing them in any ratios. The compounds of the present invention can have optically active isomers due to the presence of one or more asymmetric carbon atoms, and the present invention covers any optically active isomers and any racemates.

Some of the compounds of the present invention can be converted, by ordinary methods, to acid addition salts with hydrogen halides such as hydrofluoric acid, hydrochloric acid, hydrobromic acid and hydroiodic acid, with inorganic acids such as nitric acid, sulfuric acid, phosphoric acid, chloric acid and perchloric acid, with sulfonic acids such as methanesulfonic acid, ethanesulfonic acid, trifluoromethanesulfonic acid, benzenesulfonic acid and p-toluenesulfonic acid, with carboxylic acids such as formic acid, acetic acid, propionic acid, trifluoroacetic acid, fumaric acid, tartaric acid, oxalic acid, maleic acid, malic acid, succinic acid, benzoic acid, mandelic acid, ascorbic acid, lactic acid, gluconic acid and citric acid, with amino acids such as glutamic acid aspartic acid.

Some of the compounds of the present invention can be converted, by ordinary methods, to metal salts with alkali metals such as lithium sodium and potassium, with alkaline earth metals such as calcium, barium and magnesium, with metals such as aluminum.

Next, specific examples of each substituent used herein will be given below. n—denotes normal, i—iso, s—secondary, tert—tertiary, and Ph phenyl.

As a halogen atom in the compounds of the present invention, a fluorine atom, a chlorine atom, a bromine atom or an iodine atom may be mentioned. Herein, the expression “halo” also means such a halogen atom.

The expression Cₐ-C_{b} alkyl herein means a linear or branched hydrocarbon group containing from a to b carbon atoms such as a methyl group, an ethyl group, a n-propyl group, an i-propyl group, a n-butyl group, an i-butyl group, a s-butyl group, a t-butyl group, a n-pentyl group, a 1,1-dimethylpropyl group or a n-hexyl group, and those within the designated carbon number range are selected.

The expression Cₐ-C_{b} haloalkyl herein means a linear or branched hydrocarbon group containing from a to b carbon atoms in which hydrogen atom(s) on carbon atom(s) are optionally substituted with halogen atom(s) which may be identical with or different from one another if two or more halogen atoms are present, such as a fluoromethyl group, a chloromethyl group, a bromomethyl group, an iodomethyl group, a difluoromethyl group, a dichloromethyl group, a trifluoromethyl group, a chlorodifluoromethyl group, a trichloromethyl group, a bromodifluoromethyl group, a 2-fluoroethyl group, a 2-chloroethyl group, a 2-bromoethyl group, a 2,2-difluoroethyl group, a 2,2,2-trifluoroethyl group, a 2-chloro-2,2-difluoroethyl group, a 2,2,2-trichloroethyl group, a 1,1,2,2-tetrafluoroethyl group, a 2-chloro-1,1,2-trifluoroethyl group, a pentafluoroethyl group, a 3,3,3-trifluoropropyl group, a 2,2,3,3,3-pentafluoropropyl group, a 1,1,2,3,3,3-hexafluoropropyl group, a heptafluoropropyl group, a 2,2,2-trifluoro-1-(trifluoromethyl)ethyl group, a 1,2,2,2-tetrafluoro-1-(trifluoromethyl)ethyl group, a 2,2,3,3,4,4,4-heptafluorobutyl group or a nonafluorobutyl group, and those within the designated carbon number range are selected.

The expression Cₐ-C_{b} cycloalkyl herein means a cyclic hydrocarbon group containing from a to b carbon atoms in the form of a 3- to 6-membered monocyclic or polycyclic ring which may optionally be substituted with an alkyl group as long as the number of carbon atoms does not exceed the designated carbon number range, such as a cyclopropyl group, a 1-methylcyclopropyl group, a 2-methylcyclopropyl group, a 2,2-dimethylcyclopropyl group, a cyclobutyl group, a cyclopentyl group or a cyclohexyl group, and those within the designated carbon number range are selected.

The expression Cₐ-C_{b} halocycloalkyl means a cyclic hydrocarbon group containing from a to b carbon atoms in the form of a 3- to 6-membered monocyclic or polycyclic ring which may optionally be substituted with an alkyl group as long as the number of carbon atoms does not exceed the designated carbon number range, in which hydrogen atom(s) on carbon atom(s) in a ring moiety and/or in a side chain are optionally substituted with halogen atom(s) which may be identical with or different from one another if two or more halogen atoms are present, such as a 2,2-difluorocyclopropyl group, a 2,2-dichlorocyclopropyl group, a 2,2-dibromocyclopropyl group, a 2,2-difluoro-methylcyclopropyl group, a 2,2-dichloro-1-methylcyclopropyl group, a 2,2-dibromo-1-methylcyclopropyl group or a 2,2,3,3-tetrafluorocyclobutyl group, and those within the designated carbon atom range are selected.

The expression Cₐ-C_{b} alkenyl herein means a linear or branched unsaturated hydrocarbon group containing from a to b carbon atoms and having one or more double bonds in the molecule such as a vinyl group, a 1-propenyl group, a 2-propenyl group, a 1-methylethenyl group, a 2-butenyl group, a 2-methyl-2-propenyl group, a 3-methyl-2-butenyl group or a 1,1-dimethyl-2-propenyl group, and those within the designated carbon atom range are selected.

The expression Cₐ-C_{b} haloalkenyl herein means a linear or branched unsaturated hydrocarbon group containing from a to b carbon atoms and having one or more double bonds in the molecule, in which hydrogen atom(s) on carbon atom(s) are optionally substituted with halogen atom(s) which may be identical with or different from one another if two or more halogen atoms are present, such as a 2,2-dichlorovinyl group, a 2-fluoro2-propenyl group, a 2-chloro-2-propenyl group, a 3-chloro-2-propenyl group, a 2-bromo-2-propenyl group, a 3,3-difluoro-2-propenyl group, a 2,3-dichloro-2-propenyl group, a 3,3-dichloro-2-propenyl group, a 2,3,3-trifluoro-2-propenyl group, a 2,3,3-trichloro-2-propenyl group, a 1-(trifluoromethyl)ethenyl group, a 4,4-difluoro-3-butenyl group, a 3,4,4-trifluoro-3-butenyl group or a 3-chloro-4,4,4-trifluoro-2-butenyl group, and those within the designated carbon atom range are selected.

The expression Cₐ-C_{b} cycloalkenyl herein means a cyclic unsaturated hydrocarbon group containing from a to b carbon atoms and containing one or more end or exo double bonds in the form of a 3- to 6-membered monocyclic or polycyclic ring which may optionally be substituted with an alkyl group as long as the number of carbon atoms does not exceed the designated carbon number range, such as a 1-cyclopenten-1-yl group, a 2-cyclopenten-1-yl group, a 1-cyclohexen-1-yl group or a 2-cyclohexen-1-yl group, and those within the designated carbon atom range are selected.

The expression Cₐ-C_{b} alkylidene herein means a linear or branched hydrocarbon group containing from a to b carbon atoms which attaches by a double bond, such as a methylidene group, an ethylidene group, a propylidene group or a 1-methylethylidene group, and those within the designated carbon number range are selected.

The expression Cₐ-C_{b} alkynyl herein means a linear or branched unsaturated hydrocarbon group containing from a to b carbon atoms and having one or more triple bonds in the molecule such as an ethynyl group, a 1-propynyl group, a 2-propynyl group, a 1-butynyl group, a 2-butynyl group, a 3-butynyl group or a 1,1-dimethyl-2-propynyl group, and those within the designated carbon atom range are selected.

The expression Cₐ-C_{b} haloalkynyl herein means a linear or branched unsaturated hydrocarbon group containing from a to b carbon atoms and having one or more triple bonds in the molecule, in which hydrogen atom(s) on carbon atom(s) are optionally substituted with halogen atom(s) which may be identical with or different from one another if two or more halogen atoms are present, such as a 2-chloroethynyl group, a 2-bromoethynyl group, a 2-iodoethynyl group, a 3-chloro-2-propynyl group or a 3-bromo-2-propynyl group, a 3-iodo-2-propynyl group, and those within the designated carbon number range are selected.

The expression Cₐ-C_{b} alkoxy herein means an alkyl-O— group in which the alkyl is a previously mentioned alkyl group containing from a to b carbon atoms, such as a methoxy group, an ethoxy group, a n-propyloxy group, an i-propyloxy group, a n-butyloxy group, an i-butyloxy group, a s-butyloxy group or a tert-butyloxy group, and those within the designated carbon atom range are selected.

The expression Cₐ-C_{b} haloalkoxy herein means a haloalkyl-O— group in which the haloalkyl is a previously mentioned haloalkyl group containing from a to b carbon atoms, such as a difluoromethoxy group, a trifluoromethoxy group, a chlorodifluoromethoxy group, a bromodifluoromethoxy group, a 2-fluoroethoxy group, a 2-chloroethoxy group, a 2,2,2-trifluoroethoxy group, a 1,1,2,2-tetrafluoroethoxy group, a 2-chloro-1,1,2-trifluoroethoxy group or a 1,1,2,3,3,3-hexafluoropropyloxy group, and those within the designated carbon atom range are selected.

The expression Cₐ-C_{b} alkylthio herein means an alkyl-S— group in which the alkyl is a previously mentioned alkyl group containing from a to b carbon atoms, such as a methylthio group, an ethylthio group, a n-propylthio group, an i-propylthio group, a n-butylthio group, an i-butylthio group, a s-butylthio group or a tert-butylthio group, and those within the designated carbon atom range are selected.

The expression Cₐ-C_{b} haloalkylthio herein means a haloalkyl-S— group in which the haloalkyl is a previously mentioned haloalkyl group containing from a to b carbon atoms, such as a difluoromethylthio group, a trifluoromethylthio group, a chlorodifluoromethylthio group, a bromodifluoromethylthio group, a 2,2,2-trifluoroethylthio group, a 1,1,2,2-tetrafluoroethylthio group, a 2-chloro-1,1,2-trifluoroethylthio group, a pentafluoroethylthio group, a 1,1,2,3,3,3-hexafluoropropylthio group, a heptafluoropropylthio group, a 1,2,2,2-tetrafluoro-1-(trifluoromethyl)ethylthio group or a nonafluorobutylthio group, and those within the designated carbon atom range are selected.

The expression Cₐ-C_{b} alkylsulfinyl herein means an alkyl-S(O)— group in which the alkyl is a previously mentioned alkyl group containing from a to b carbon atoms, such as a methylsulfinyl group, an ethylsulfinyl group, a n-propylsulfinyl group, an i-propylsulfinyl group, a n-butylsulfinyl group, an i-butylsulfinyl group, a s-butylsulfinyl group or a tert-butylsulfinyl group, and those within the designated carbon atom range are selected.

The expression Cₐ-C_{b} haloalkylsulfinyl herein means a haloalkyl-S(O)— group in which the haloalkyl is a previously mentioned haloalkyl group containing from a to b carbon atoms, such as a difluoromethylsulfinyl group, a trifluoromethylsulfinyl group, a chlorodifluoromethylsulfinyl group, a bromodifluoromethylsulfinyl group, a 2,2,2-trifluoroethylsulfinyl group, a 1,2,2,2-tetrafluoro-1-(trifluoromethyl)ethylsulfinyl group or a nonafluorobutylsulfinyl group, and those within the designated carbon atom range are selected.

The expression Cₐ-C_{b} alkylsulfonyl herein means an alkyl-SO₂— group in which the alkyl is a previously mentioned alkyl group containing from a to b carbon atoms, such as a methylsulfonyl group, an ethylsulfonyl group, a n-propylsulfonyl group, an i-propylsulfonyl group, a n-butylsulfonyl group, an i-butylsulfonyl group, a s-butylsulfonyl group or a tert-butylsulfonyl group, and those within the designated carbon atom range are selected.

The expression Cₐ-C_{b} haloalkylsulfonyl herein means a haloalkyl-SO₂— group in which the haloalkyl is a previously mentioned haloalkyl group containing from a to b carbon atoms, such as a difluoromethylsulfonyl group, a trifluoromethylsulfonyl group, a chlorodifluoromethylsulfonyl group, a bromodifluoromethylsulfonyl group, a 2,2,2-trifluoroethylsulfonyl group, a 1,1,2,2-tetrafluoroethylsulfonyl group or a 2-chloro-1,1,2-trifluoroethylsulfonyl group, and those within the designated carbon atom range are selected.

The expression Cₐ-C_{b} alkylamino herein means an amino group in which either hydrogen atom is replaced by a previously mentioned alkyl group containing from a to b carbon atoms, such as a methylamino group, an ethylamino group, a n-propylamino group, an i-propylamino group, a n-butylamino group, an i-butylamino group or a tert-butylamino group, and those within the designated carbon atom range are selected.

The expression di(Cₐ-C_{b} alkyl)amino herein means an amino group in which both hydrogen atoms are replaced by previously mentioned alkyl groups containing from a to b carbon atoms which may be identical with or different from each other, such as a dimethylamino group, an ethyl(methyl)amino group, a diethylamino group, a n-propyl(methyl)amino group, an i-propyl(methyl)amino group, a di(n-propyl)amino group or a di(n-butyl)amino group, and those within the designated carbon atom range are selected.

The expression Cₐ-C_{b} alkylimino herein means an alkyl-N═ group in which the alkyl means a previously mentioned alkyl group containing from a to b carbon atoms, such as a methylimino group, an ethylimino group, a n-propylimino group, an i-propylimino group, a n-butylimino group, an i-butylimino group or a s-butylimino group, and those within the designated carbon atom range are selected.

The expression Cₐ-C_{b} alkoxyimino herein means an alkoxy-N═ group in which the alkoxy means a previously mentioned alkoxy group containing from a to b carbon atoms, such as a methoxyimino group, an ethoxyimino group, a n-propyloxyimino group, an i-propyloxyimino group or a n-butyloxyimino group, and those within the designated carbon atom range are selected.

The expression Cₐ-C_{b} alkylcarbonyl herein means an alkyl-C(O)— group in which the alkyl means a previously mentioned alkyl group containing from a to b carbon atoms, such as an acetyl group, a propionyl group, a butyryl group, an isobutyryl group, a valeryl group, an isovaleryl group, a 2-methylbutanoyl group, a pivaloyl group, a hexanoyl group or a heptanoyl group, and those within the designated carbon atom range are selected.

The expression Cₐ-C_{b} haloalkylcarbonyl herein means a haloalkyl-C(O)— group in which the haloalkyl means a previously mentioned haloalkyl group containing from a to b carbon atoms, such as a fluoroacetyl group, a chloroacetyl group, a difluoroacetyl group, a dichloroacetyl group, a trifluoroacetyl group, a chlorodifluoroacetyl group, a bromodifluoroacetyl group, a trichloroacetyl group, a pentafluoropropionyl group, a heptafluorobutanoyl group or a 3-chloro-2,2-dimethylpropanoyl group, and those within the designated carbon atom range are selected.

The expression Cₐ-C_{b} cycloalkylcarbonyl herein means a cycloalkyl-C(O)— group in which the cycloalkyl means a previously mentioned cycloalkyl group containing from a to b carbon atoms, such as a cyclopropylcarbonyl group, a 2-methylcyclopropylcarbonyl group or a cyclobutylcarbonyl group, and those within the designated carbon atom range are selected.

The expression Cₐ-C_{b} alkoxycarbonyl herein means an alkyl-O—C(O)— group in which the alkyl means a previously mentioned alkyl group containing from a to b carbon atoms, such as a methoxycarbonyl group, an ethoxycarbonyl group, a n-propyloxycarbonyl group, an i-propyloxycarbonyl group, a n-butoxycarbonyl group, an i-butoxycarbonyl group or a tert-butoxycarbonyl group, and those within the designated carbon atom range are selected.

The expression Cₐ-C_{b} haloalkoxycarbonyl herein means a haloalkyl-O—C(O)— group in which the haloalkyl means a previously mentioned haloalkyl group containing from a to b carbon atoms, such as a chloromethoxycarbonyl group, a 2-chloroethoxycarbonyl group, a 2,2-difluoroethoxycarbonyl group, a 2,2,2-trifluoroethoxycarbonyl group or a 2,2,2-trichloroethoxycarbonyl group, and those within the designated carbon atom range are selected.

The expression Cₐ-C_{b} alkylaminocarbonyl herein means a carbamoyl group in which either hydrogen atom is replaced by a previously mentioned alkyl group containing from a to b carbon atoms, such as a methylcarbamoyl group, an ethylcarbamoyl group, a n-propylcarbamoyl group, an i-propylcarbamoyl group, a n-butylcarbamoyl group, an i-butylcarbamoyl group, a s-butylcarbamoyl group or a tert-butylcarbamoyl group, and those within the designated carbon atom range are selected.

The expression Cₐ-C_{b} haloalkylaminocarbonyl herein means a carbamoyl group in which either hydrogen atom is replaced by a previously mentioned haloalkyl group containing from a to b carbon atoms, such as a 2-fluoroethylcarbamoyl group, a 2-chloroethylcarbamoyl group, a 2,2-difluoroethylcarbamoyl group or a 2,2,2-trifluoroethylcarbamoyl group, and those within the designated carbon atom range are selected.

The expression di(Cₐ-C_{b} alkyl)aminocarbonyl herein means a carbamoyl group in which both hydrogen atoms are replaced by previously mentioned alkyl groups containing from a to b carbon atoms which may be identical with or different from each other, such as an N,N-dimethylcarbamoyl group, an N-ethyl-N-methylcarbamoyl group, an N,N-diethylcarbamoyl group, an N,N-di(n-propyl)carbamoyl group or an N,N-di(n-butyl)carbamoyl group, and those within the designated carbon atom range are selected.

The expression Cₐ-C_{b} alkylaminosulfonyl herein means a sulfamoyl group in which either hydrogen atom is replaced by a previously mentioned alkyl group containing from a to b carbon atoms, such as a methylsulfamoyl group, an ethylsulfamoyl group, a n-propylsulfamoyl group, an i-propylsulfamoyl group, a n-butylsulfamoyl group, an i-butylsulfamoyl group, a s-butylsulfamoyl group or a tert-butylsulfamoyl group, and those within the designated carbon atom range are selected.

The expression di(Cₐ-C_{b} alkyl)aminosulfonyl herein means a sulfamoyl group in which both hydrogen atoms are replaced by previously mentioned alkyl groups containing from a to b carbon atoms which may be identical with or different from each other, such as an N,N-dimethylsulfamoyl group, an N-ethyl-N-methylsulfamoyl group, an N,N-diethylsulfamoyl group, an N,N-di(n-propyl)sulfamoyl group or an N,N-di(n-butyl)sulfamoyl group, and those within the designated carbon atom range are selected.

The expression such as (Cₐ-C_{b}) alkyl optionally substituted with R⁵, (Cₐ-C_{b}) alkyl optionally substituted with R¹⁴, (Cₐ-C_{b}) alkyl optionally substituted with R^{14a}, (Cₐ-C_{b}) alkyl optionally substituted with R¹⁹, (Cₐ-C_{b}) alkyl optionally substituted with R²⁸, (Cₐ-C_{b}) alkyl optionally substituted with R^{28a}, (Cₐ-C_{b}) alkyl optionally substituted with R³² or (Cₐ-C_{b}) alkyl optionally substituted with R^{32a} herein means a previously mentioned alkyl group having from a to b carbon atoms in which hydrogen atom(s) on carbon atom(s) are optionally substituted with optional R⁵, R¹⁴, R^{14a}, R¹⁹, R²⁸, R^{28a}, R³² or R^{32a}, and those within the designated carbon atom range are selected. When there are two or more R⁵'s, R¹⁴'s, R^{14a}'s, R¹⁹'s, R²⁸'s, R^{28a}'s, R³²'s or R^{32a}'s on a (Cₐ-C_{b}) alkyl group, each R⁵, R¹⁴, R^{14a}, R¹⁹, R²⁸, R^{28a}, R³² or R^{32a} may be identical with or different from one another.

The expression such as (Cₐ-C_{b}) cycloalkyl optionally substituted with R⁵, (Cₐ-C_{b}) cycloalkyl optionally substituted with R¹⁴, (Cₐ-C_{b}) cycloalkyl optionally substituted with R^{14a}, (Cₐ-C_{b}) cycloalkyl optionally substituted with R¹⁹, (Cₐ-C_{b}) cycloalkyl optionally substituted with R²⁸, (Cₐ-C_{b}) cycloalkyl optionally substituted with R^{28a} or (Cₐ-C_{b}) cycloalkyl optionally substituted with R³² herein means a previously mentioned cycloalkyl group having from a to b carbon atoms in which hydrogen atom(s) on carbon atom(s) in the ring moiety and/or in the side chain are optionally substituted with (Cₐ-C_{b}) cycloalkyl optionally substituted with optional R⁵, R¹⁴, R^{14a}, R¹⁹, R²⁸, R^{28a} or R³², and those within the designated carbon atom range are selected. When there are two or more R⁵'s, R¹⁴'s, R^{14a}'s, R¹⁹'s, R²⁸'s, R^{28a}'s or R³²'s on a (Cₐ-C_{b}) cycloalkyl group, each R⁵, R¹⁴, R^{14a}, R¹⁹, R²⁸, R^{28a} or R³² may be identical with or different from one another.

The expression such as (Cₐ-C_{b}) alkenyl optionally substituted with R⁵, (Cₐ-C_{b}) alkenyl optionally substituted with R¹⁴, (Cₐ-C_{b}) alkenyl optionally substituted with R^{14a} (Cₐ-C_{b}) alkenyl optionally substituted with R¹⁹, (Cₐ-C_{b}) alkenyl optionally substituted with R²⁸, (Cₐ-C_{b}) alkenyl optionally substituted with R^{28a} or (Cₐ-C_{b}) alkenyl optionally substituted with R³² herein means a previously mentioned alkenyl group having from a to b carbon atoms in which hydrogen atom(s) on carbon atom(s) are optionally substituted with optional R⁵, R¹⁴, R^{14a}, R¹⁹, R²⁸, R^{28a} or R³², and those within the designated carbon atom range are selected. When there are two or more R⁵'s, R¹⁴'s, R^{14a}'s, R¹⁹'s, R²⁸'s, R^{28a}'s or R³²'s on a (Cₐ-C_{b}) alkenyl group, each R⁵, R¹⁴, R^{14a}, R¹⁹, R²⁸, R^{28a} or R³² may be identical with or different from one another.

The expression such as (Cₐ-C_{b}) cycloalkenyl optionally substituted with R⁵, (Cₐ-C_{b}) cycloalkenyl optionally substituted with R¹⁴, (Cₐ-C_{b}) cycloalkenyl optionally substituted with R^{14a}, (Cₐ-C_{b}) cycloalkenyl optionally substituted with R¹⁹, (Cₐ-C_{b}) cycloalkenyl optionally substituted with R²⁸, (Cₐ-C_{b}) cycloalkenyl optionally substituted with R^{28a} or (Cₐ-C_{b}) cycloalkenyl optionally substituted with R³² herein mean a previously mentioned cycloalkenyl group having from a to b carbon atoms in which hydrogen atom(s) on carbon atom(s) in the ring moiety and/or in the side chain are optionally substituted with optional R⁵, R¹⁴, R^{14a}, R¹⁹, R²⁸, R^{28a} or R³², and those within the designated carbon atom range are selected. When there are two or more R⁵'s, R¹⁴'s, R^{14a}'s, R¹⁹'s, R²⁸'s, R^{28a}'s or R³²'s on a (Cₐ-C_{b}) cycloalkenyl group, each R⁵, R¹⁴, R^{14a}, R¹⁹, R²⁸, R^{28a} or R³² may be identical with or different from one another.

The expression such as (Cₐ-C_{b}) alkynyl optionally substituted with R⁵, (Cₐ-C_{b}) alkynyl optionally substituted with R¹⁴, (Cₐ-C_{b}) alkynyl optionally substituted with R^{14a} (Cₐ-C_{b}) alkynyl optionally substituted with R¹⁹, (Cₐ-C_{b}) alkynyl optionally substituted with R²⁸, (Cₐ-C_{b}) alkynyl optionally substituted with R^{28a} or (Cₐ-C_{b}) alkynyl optionally substituted with R³² herein means a previously mentioned alkynyl group having from a to b carbon atoms in which hydrogen atom(s) on carbon atom(s) are optionally substituted with optional R⁵, R¹⁴, R^{14a}, R¹⁹, R²⁸, R^{28a} or R³², and those within the designated carbon atom range are selected. When there are two or more R⁵'s, R¹⁴'s, R^{14a}'s, R¹⁹'s, R²⁸'s, R^{28a}'s or R³²'s on a (Cₐ-C_{b}) alkynyl group, each R⁵, R¹⁴, R^{14a}, R¹⁹, R²⁸, R^{28a} or R³² may be identical with or different from one another.

The expression such as “R⁸ may form, together with R^{8a}, a C₂-C₇ alkylene chain to form a 3 to 8-membered ring together with the nitrogen atom attached to R⁸ and R^{8a} wherein the alkylene chain may contain an oxygen atom, sulfur atom or nitrogen atom”, “R¹³ and R^{13a} may form, together with each other, a C₂-C₇ alkylene chain to form a 3 to 8-membered ring together with the nitrogen atom attached to R¹³ and R^{13a}, wherein the alkylene chain may contain an oxygen atom, sulfur atom or nitrogen atom” or “R¹⁷ and R^{17a} may form, together with each other, a C₂-C₇ alkylene chain to form a 3 to 8-membered ring together with the nitrogen atom attached to R¹⁷ and R^{17a}, wherein the alkylene chain may contain an oxygen atom, sulfur atom or nitrogen atom” is specifically exemplified by aziridine, azetidine, azetidin-2-one, pyrrolidine, pyrrolidin-2-one, oxazolidine, oxazolidin-2-one, oxazolidine-2-thione, thiazolidine, thiazolidin-2-one, thiazolidine-2-thione, imidazolidine, imidazolidin-2-one, imidazolidine-2-thione, piperidine, piperidin-2-one, piperidine-2-thione, 2H-3,4,5,6-tetrahydro-1,3-oxazin-2-one, 2H-3,4,5,6-tetrahydro-1,3-oxazine-2-thione, morpholine, 2H-3,4,5,6-tetrahydro-1,3-thiazin-2-one, 2H-3,4,5,6-tetrahydro-1,3-thiazine-2-thione, thiomorpholine, perhydropyrimidin-2-one, piperazine, homopiperidine, homopiperidin-2-one, heptamethylenimine or the like, and those within the designated carbon atom range are selected.

The expression “R^{b3} may form, together with R^{b2}, a C₄-C₆ alkylene chain or a C₄-C₆ alkenylene chain to form a 5 to 7-membered ring together with the carbon atom attached to R^{b3} and R^{b2}, wherein the alkylene chain or the alkenylene chain may contain from 1 to 3 oxygen atoms, sulfur atoms or nitrogen atoms” is specifically exemplified by thiazolidin-2-ylidene, 2,3-dihydrothiazol-2-ylidene, imidazolidin-2-ylidene, 2,3-dihydroimidazol-2-ylidene, 2,3-dihydro-1,3,4-thiadiazol-2-ylidene, 1,2-dihydropyridin-2-ylidene, 2,3-dihydropyridazin-3-ylidene, 1,2-dihydropyrazin-2-ylidene, 1,2-dihydropyrimidin-2-ylidene, 6H-2,3-dihydro-1,3,4-thiadiazin-2-ylidene or the like, and those within the designated carbon atom range are selected.

The expression “R⁷ may form, together with R^{a}, a C₂-C₆ alkylene chain or a C₂-C₆ alkenylene chain containing a double bond to form a 4 to 8-membered ring together with the carbon atom attached to R⁷ and the nitrogen atom attached to R^{a}, wherein the alkylene chain or the alkenylene chain may contain one or two oxygen atoms, sulfur atoms or nitrogen atoms” is specifically exemplified by pyrrolidin-2-one, pyrrolidine-2-thione, 1H-pyrrol-2(5H)-one, pyrrolidine-2,5-dione, pyrrolidin-2-imine, piperidin-2-one, piperidine-2-thione, piperidin-2-imine, 5,6-dihydropyridin-2(1H)-one, 1,3-oxazinan-2-one, 1,3-oxazinane-2-thione, 1,3-oxazinan-4-one, 1,3-thiazinan-4-one, tetrahydropyrimidin-4(1H)-one, piperidine-2,3-dione, piperidine-2,6-dione or the like, and those within the designated carbon atom range are selected.

The expression “R^{8a} may form, together with R^{a}, a C₂-C₅ alkylene chain to form a 5 to 8-membered ring together with the nitrogen atom attached to R^{8a} and the nitrogen atom attached to R^{a}, wherein the alkylene chain may contain an oxygen atom, a sulfur atom or a nitrogen atom” is specifically exemplified by imidazolidin-2-one, imidazolidine-2-thione, imidazolidin-2-imine, imidazolidine-2,4-dione, tetrahydropyrimidin-2(1H)-one, tetrahydropyrimidine-2(1H)-thione, tetrahydropyrimidin-2(1H)-imine, 1,2,4-oxazinan-3-one, 1,3,5-oxazinan-4-one, 1,3,5-thiazinan-4-one, 1,3,5-triazinan-2-one, dihydropyrimidine-2,4(1H,3H)-dione or the like, and those within the designated carbon atom range are selected.

As the scope of the substituent represented by R³ in the compounds which fall within the present invention, the following sets may, for example, be mentioned.

R³I: a hydrogen atom, a halogen atom, C₁-C₆ alkyl.

R³II: a halogen atom, C₁-C₆ alkyl.

R³III: a halogen atom.

As the scope of the substituent represented by R^{a} in the compounds which fall within the present invention, the following sets may, for example, be mentioned.

R^{a}I: C₁-C₆ alkyl, (C₁-C₆) alkyl optionally substituted with R⁵ [wherein R⁵ is —OR¹¹, and R¹¹ is C₁-C₆ alkyl], —C(═NR^{8b})R^{7a} [wherein R^{7a} is a hydrogen atom or —C₁-C₆ alkyl, R^{8b} is —OR¹¹, and R¹¹ is C₁-C₆ alkyl], (C₁-C₆) alkyl optionally substituted with R⁵ [wherein R⁵ is —C(═NOR¹¹)R¹², and each of R¹¹ and R¹² is independently C₁-C₆ alkyl], (C₁-C₆) alkyl optionally substituted with R⁵ [wherein R⁵ is —Si(R^{9a})(R^{9b})R⁹, and each of R⁹, R^{9a} and R^{9b} is independently C₁-C₆ alkyl].

R^{a}II: (C₁-C₆) alkyl optionally substituted with R⁵ [wherein R⁵ is —OR¹¹, and R¹¹ is C₁-C₆ alkyl].

R^{a}III: C₁-C₆ alkyl.

R^{a}IV: —C(═NR^{8b})R^{7a} [wherein R^{7a} is a hydrogen atom or —C₁-C₆ alkyl, R^{8b} is —OR¹¹, and R¹¹ is C₁-C₆ alkyl].

R^{a}V: (C₁-C₆) alkyl optionally substituted with R⁵ [wherein R⁵ is —C(═NOR¹¹)R¹², and each of R¹¹ and R¹² is independently C₁-C₆ alkyl].

R^{a}VI: (C₁-C₆) alkyl optionally substituted with R⁵ [wherein R⁵ is —Si(R^{9a})(R^{9b})R⁹, and each of R⁹, R^{9a} and R^{9b} is independently C₁-C₆ alkyl].

R^{a}VII: (C₁-C₆) alkyl optionally substituted with R⁵ [wherein R⁵ is —S(O)ᵣR¹¹, R¹¹ is C₁-C₆ alkyl, and r is an integer of from 0 to 2].

R^{a}VIII: (C₁-C₆) alkyl optionally substituted with R⁵ [wherein R⁵ is cyano].

R^{a}IX: (C₁-C₆) alkyl optionally substituted with R⁵ [wherein R⁵ is C₃-C₈ cycloalkyl].

R^{a}X: C₂-C₁₂ alkynyl.

As the scope of the substituent represented by R^{b} in the compounds which fall within the present invention, the following sets may, for example, be mentioned.

R^{b}I: —C(O)R⁷, —C(S)R⁷, —C(O)OR^{6a} [wherein R^{6a} is C₁-C₆ alkyl], —C(O)N(R^{8a})R⁸ [wherein R⁸ is C₁-C₆ alkyl, (C₁-C₆) alkyl optionally substituted with R¹⁴ or C₃-C₈ cycloalkyl, R^{8a} is a hydrogen atom or C₁-C₆ alkyl, and R¹⁴ is a halogen atom], —C(S)N(R^{8a})R⁸ [wherein R⁸ is C₁-C₆ alkyl, (C₁-C₆) alkyl optionally substituted with R¹⁴ or C₃-C₈ cycloalkyl, R^{8a} is a hydrogen atom or C₁-C₆ alkyl, and R¹⁴ is a halogen atom].

R^{b}II: —C(S)R⁷.

R^{b}III: —C(O)R⁷.

R^{b}IV: —C(O)OR^{6a} [wherein R^{6a} is C₁-C₆ alkyl].

R^{b}V: —C(O)N(R^{8a})R⁸ [wherein R⁸ is C₁-C₆ alkyl, (C₁-C₆) alkyl optionally substituted with R¹⁴ or C₃-C₈ cycloalkyl, R^{8a} is a hydrogen atom or C₁-C₆ alkyl, and R¹⁴ is a halogen atom].

R^{b}VI: —C(S)N(R^{8a})R⁸ [wherein R⁸ is C₁-C₆ alkyl, (C₁-C₆) alkyl optionally substituted with R¹⁴ or C₃-C₈ cycloalkyl, R^{8a} is a hydrogen atom or C₁-C₆ alkyl, and R¹⁴ is a halogen atom].

R^{b}VII: R^{b} forms ═C(R^{b2})R^{b3} together with R^{a} [wherein R^{b2} is a hydrogen atom or C₁-C₁₅ alkyl, R^{b3} is —N(R^{8b})R⁸, R⁸ is —C(O)R¹², R^{8b} is C₁-C₁₅ alkyl or —OR¹¹, R¹¹ is C₁-C₆ alkyl, and R¹² is (C₁-C₆) alkyl optionally substituted with R^{14a}].

As the scope of the substituent represented by R⁷ in the compounds which fall within the present invention, the following sets may, for example, be mentioned.

R⁷I: (C₁-C₆) alkyl optionally substituted with R¹⁴ [wherein R¹⁴ is —S(O)ᵣR¹⁵, R¹⁵ is C₁-C₆ alkyl, r is an integer of from 0 to 2], —C(═NOR¹¹)R¹² [wherein R¹¹ is C₁-C₆ alkyl, R¹² is (C₁-C₆) alkyl optionally substituted with R^{14a}, R^{14a} is —S(O)ᵣR¹⁵, R¹⁵ is C₁-C₆ alkyl, and r is an integer of from 0 to 2].

R⁷II: —C(═NOR¹¹)R¹² [wherein R¹¹ is C₁-C₆ alkyl, R¹² is (C₁-C₆) alkyl optionally substituted with R^{14a}, R^{14a} is —S(O)ᵣR¹⁵, R¹⁵ is C₁-C₆ alkyl, and r is an integer of from 0 to 2].

R⁷III: (C₁-C₆) alkyl optionally substituted with R¹⁴ [wherein R¹⁴ is —S(O)ᵣR¹⁵, R¹⁵ is C₁-C₆ alkyl, and r is an integer of from 0 to 2].

R⁷IV: (C₁-C₆) alkyl optionally substituted with R¹⁴ [wherein R¹⁴ is D1-82, D1-87, D1-93 or D1-98, and r is an integer of from 0 to 2].

R⁷V: (C₁-C₆) alkyl optionally substituted with R¹⁴ [wherein R¹⁴ is D1-82 or D1-93, and r is an integer of from 0 to 2].

R⁷VI: (C₁-C₆) alkyl optionally substituted with R¹⁴ [wherein R¹⁴ is D1-87 or D1-98].

R⁷VII: (C₁-C₆) alkyl optionally substituted with R¹⁴ [wherein R¹⁴ is —S(O)ᵣR¹⁵, R¹⁵ is D1-32, and r is an integer of from 0 to 2].

R⁷VIII: (C₁-C₆) alkyl optionally substituted with R¹⁴ [wherein R¹⁴ is —S(═NR^{17b})R^{15a} or —S(O)(═NR^{17b})R^{15a}, R^{15a} is C1-C6 alkyl, and R^{17b} is cyano].

R⁷IX: (C₁-C₆) alkyl optionally substituted with R¹⁴ [wherein R¹⁴ is —C(O)N(R^{17a})R¹⁷, R¹⁷ is (C₁-C₆) alkyl optionally substituted with R¹⁹ or —S(O)ᵣR²⁰, R^{17a} is a hydrogen atom or —C₁-C₆ alkyl, R¹⁹ is a halogen atom, R²⁰ is C₁-C₆ alkyl, (C₁-C₆) alkyl optionally substituted with R³², and R³² is a halogen atom].

The sets indicating the scope of each substituent in the compounds which fall within the present invention may be combined arbitrarily to indicate the scope of the compounds of the present invention. The scope of R³, R^{a}, R^{b} or R⁷ may be combined, for example, as shown in Table 1. The combinations shown in Table 1 merely exemplify the present invention, and the present invention is by no means restricted thereto.

TABLE 1

| R³ | R^{a} | R^{b} | R⁷ |
|---|---|---|---|
| R³I | R^{a}I | R^{b}I | R⁷I |
| R³I | R^{a}I | R^{b}I | R⁷II |
| R³I | R^{a}I | R^{b}I | R⁷III |
| R³I | R^{a}I | R^{b}I | R⁷IV |
| R³I | R^{a}I | R^{b}I | R⁷V |
| R³I | R^{a}I | R^{b}I | R⁷VI |
| R³I | R^{a}I | R^{b}I | R⁷VII |
| R³I | R^{a}I | R^{b}I | R⁷VIII |
| R³I | R^{a}I | R^{b}I | R⁷IX |
| R³I | R^{a}I | R^{b}II | R⁷I |
| R³I | R^{a}I | R^{b}II | R⁷II |
| R³I | R^{a}I | R^{b}II | R⁷III |
| R³I | R^{a}I | R^{b}II | R⁷IV |
| R³I | R^{a}I | R^{b}II | R⁷V |
| R³I | R^{a}I | R^{b}II | R⁷VI |
| R³I | R^{a}I | R^{b}II | R⁷VII |
| R³I | R^{a}I | R^{b}II | R⁷VIII |
| R³I | R^{a}I | R^{b}II | R⁷IX |
| R³I | R^{a}I | R^{b}III | R⁷I |
| R³I | R^{a}I | R^{b}III | R⁷II |
| R³I | R^{a}I | R^{b}III | R⁷III |
| R³I | R^{a}I | R^{b}III | R⁷IV |
| R³I | R^{a}I | R^{b}III | R⁷V |
| R³I | R^{a}I | R^{b}III | R⁷VI |
| R³I | R^{a}I | R^{b}III | R⁷VII |
| R³I | R^{a}I | R^{b}III | R⁷VIII |
| R³I | R^{a}I | R^{b}III | R⁷XI |
| R³I | R^{a}I | R^{b}IV | — |
| R³I | R^{a}I | R^{b}V | — |
| R³I | R^{a}I | R^{b}VI | — |
| R³I | R^{a}II | R^{b}I | R⁷I |
| R³I | R^{a}II | R^{b}I | R⁷II |
| R³I | R^{a}II | R^{b}I | R⁷III |
| R³I | R^{a}II | R^{b}I | R⁷IV |
| R³I | R^{a}II | R^{b}I | R⁷V |
| R³I | R^{a}II | R^{b}I | R⁷VI |
| R³I | R^{a}II | R^{b}I | R⁷VII |
| R³I | R^{a}II | R^{b}I | R⁷VIII |
| R³I | R^{a}II | R^{b}I | R⁷IX |
| R³I | R^{a}II | R^{b}II | R⁷I |
| R³I | R^{a}II | R^{b}II | R⁷II |
| R³I | R^{a}II | R^{b}II | R⁷III |
| R³I | R^{a}II | R^{b}II | R⁷IV |
| R³I | R^{a}II | R^{b}II | R⁷V |
| R³I | R^{a}II | R^{b}II | R⁷VI |
| R³I | R^{a}II | R^{b}II | R⁷VII |
| R³I | R^{a}II | R^{b}II | R⁷VIII |
| R³I | R^{a}II | R^{b}II | R⁷IX |
| R³I | R^{a}II | R^{b}III | R⁷I |
| R³I | R^{a}II | R^{b}III | R⁷II |
| R³I | R^{a}II | R^{b}III | R⁷III |
| R³I | R^{a}II | R^{b}III | R⁷IV |
| R³I | R^{a}II | R^{b}III | R⁷V |
| R³I | R^{a}II | R^{b}III | R⁷VI |
| R³I | R^{a}II | R^{b}III | R⁷VII |
| R³I | R^{a}II | R^{b}III | R⁷VIII |
| R³I | R^{a}II | R^{b}III | R⁷IX |
| R³I | R^{a}II | R^{b}IV | — |
| R³I | R^{a}II | R^{b}V | — |
| R³I | R^{a}II | R^{b}VI | — |
| R³I | R^{a}III | R^{b}I | R⁷I |
| R³I | R^{a}III | R^{b}I | R⁷II |
| R³I | R^{a}III | R^{b}I | R⁷III |
| R³I | R^{a}III | R^{b}I | R⁷IX |
| R³I | R^{a}III | R^{b}I | R⁷IV |
| R³I | R^{a}III | R^{b}I | R⁷V |
| R³I | R^{a}III | R^{b}I | R⁷VI |
| R³I | R^{a}III | R^{b}I | R⁷VII |
| R³I | R^{a}III | R^{b}I | R⁷VIII |
| R³I | R^{a}III | R^{b}II | R⁷I |
| R³I | R^{a}III | R^{b}II | R⁷II |
| R³I | R^{a}III | R^{b}II | R⁷III |
| R³I | R^{a}III | R^{b}II | R⁷IV |
| R³I | R^{a}III | R^{b}II | R⁷V |
| R³I | R^{a}III | R^{b}II | R⁷VI |
| R³I | R^{a}III | R^{b}II | R⁷VII |
| R³I | R^{a}III | R^{b}II | R⁷VIII |
| R³I | R^{a}III | R^{b}II | R⁷IX |
| R³I | R^{a}III | R^{b}III | R⁷I |
| R³I | R^{a}III | R^{b}III | R⁷II |
| R³I | R^{a}III | R^{b}III | R⁷III |
| R³I | R^{a}III | R^{b}III | R⁷IV |
| R³I | R^{a}III | R^{b}III | R⁷V |
| R³I | R^{a}III | R^{b}III | R⁷VI |
| R³I | R^{a}III | R^{b}III | R⁷VII |
| R³I | R^{a}III | R^{b}III | R⁷VIII |
| R³I | R^{a}III | R^{b}III | R⁷IX |
| R³I | R^{a}III | R^{b}IV | — |
| R³I | R^{a}III | R^{b}V | — |
| R³I | R^{a}III | R^{b}VI | — |
| R³I | R^{a}IV | R^{b}I | R⁷I |
| R³I | R^{a}IV | R^{b}I | R⁷II |
| R³I | R^{a}IV | R^{b}I | R⁷III |
| R³I | R^{a}IV | R^{b}I | R⁷IV |
| R³I | R^{a}IV | R^{b}I | R⁷V |
| R³I | R^{a}IV | R^{b}I | R⁷VI |
| R³I | R^{a}IV | R^{b}I | R⁷VII |
| R³I | R^{a}IV | R^{b}I | R⁷VIII |
| R³I | R^{a}IV | R^{b}I | R⁷IX |
| R³I | R^{a}IV | R^{b}II | R⁷I |
| R³I | R^{a}IV | R^{b}II | R⁷II |
| R³I | R^{a}IV | R^{b}II | R⁷III |
| R³I | R^{a}IV | R^{b}II | R⁷IV |
| R³I | R^{a}IV | R^{b}II | R⁷V |
| R³I | R^{a}IV | R^{b}II | R⁷VI |
| R³I | R^{a}IV | R^{b}II | R⁷VII |
| R³I | R^{a}IV | R^{b}II | R⁷VIII |
| R³I | R^{a}IV | R^{b}II | R⁷IX |
| R³I | R^{a}IV | R^{b}III | R⁷I |
| R³I | R^{a}IV | R^{b}III | R⁷II |
| R³I | R^{a}IV | R^{b}III | R⁷III |
| R³I | R^{a}IV | R^{b}III | R⁷IV |
| R³I | R^{a}IV | R^{b}III | R⁷V |
| R³I | R^{a}IV | R^{b}III | R⁷VI |
| R³I | R^{a}IV | R^{b}III | R⁷VII |
| R³I | R^{a}IV | R^{b}III | R⁷VIII |
| R³I | R^{a}IV | R^{b}III | R⁷IX |
| R³I | R^{a}IV | R^{b}IV | — |
| R³I | R^{a}IV | R^{b}V | — |
| R³I | R^{a}IV | R^{b}VI | — |
| R³I | R^{a}V | R^{b}I | R⁷I |
| R³I | R^{a}V | R^{b}I | R⁷II |
| R³I | R^{a}V | R^{b}I | R⁷III |
| R³I | R^{a}V | R^{b}I | R⁷IV |
| R³I | R^{a}V | R^{b}I | R⁷V |
| R³I | R^{a}V | R^{b}I | R⁷VI |
| R³I | R^{a}V | R^{b}I | R⁷VII |
| R³I | R^{a}V | R^{b}I | R⁷VIII |
| R³I | R^{a}V | R^{b}I | R⁷IX |
| R³I | R^{a}V | R^{b}II | R⁷I |
| R³I | R^{a}V | R^{b}II | R⁷II |
| R³I | R^{a}V | R^{b}II | R⁷III |
| R³I | R^{a}V | R^{b}II | R⁷IV |
| R³I | R^{a}V | R^{b}II | R⁷V |
| R³I | R^{a}V | R^{b}II | R⁷VI |
| R³I | R^{a}V | R^{b}II | R⁷VII |
| R³I | R^{a}V | R^{b}II | R⁷VIII |
| R³I | R^{a}V | R^{b}II | R⁷IX |
| R³I | R^{a}V | R^{b}III | R⁷I |
| R³I | R^{a}V | R^{b}III | R⁷II |
| R³I | R^{a}V | R^{b}III | R⁷III |
| R³I | R^{a}V | R^{b}III | R⁷IV |
| R³I | R^{a}V | R^{b}III | R⁷V |
| R³I | R^{a}V | R^{b}III | R⁷VI |
| R³I | R^{a}V | R^{b}III | R⁷VII |
| R³I | R^{a}V | R^{b}III | R⁷VIII |
| R³I | R^{a}V | R^{b}III | R⁷IX |
| R³I | R^{a}V | R^{b}IV | — |
| R³I | R^{a}V | R^{b}V | — |
| R³I | R^{a}V | R^{b}VI | — |
| R³I | R^{a}VI | R^{b}I | R⁷I |
| R³I | R^{a}VI | R^{b}I | R⁷II |
| R³I | R^{a}VI | R^{b}I | R⁷III |
| R³I | R^{a}VI | R^{b}I | R⁷IV |
| R³I | R^{a}VI | R^{b}I | R⁷V |
| R³I | R^{a}VI | R^{b}I | R⁷VI |
| R³I | R^{a}VI | R^{b}I | R⁷VII |
| R³I | R^{a}VI | R^{b}I | R⁷VIII |
| R³I | R^{a}VI | R^{b}I | R⁷IX |
| R³I | R^{a}VI | R^{b}II | R⁷I |
| R³I | R^{a}VI | R^{b}II | R⁷II |
| R³I | R^{a}VI | R^{b}II | R⁷III |
| R³I | R^{a}VI | R^{b}II | R⁷IV |
| R³I | R^{a}VI | R^{b}II | R⁷V |
| R³I | R^{a}VI | R^{b}II | R⁷VI |
| R³I | R^{a}VI | R^{b}II | R⁷VII |
| R³I | R^{a}VI | R^{b}II | R⁷VIII |
| R³I | R^{a}VI | R^{b}II | R⁷IX |
| R³I | R^{a}VI | R^{b}III | R⁷I |
| R³I | R^{a}VI | R^{b}III | R⁷II |
| R³I | R^{a}VI | R^{b}III | R⁷III |
| R³I | R^{a}VI | R^{b}III | R⁷IV |
| R³I | R^{a}VI | R^{b}III | R⁷V |
| R³I | R^{a}VI | R^{b}III | R⁷VI |
| R³I | R^{a}VI | R^{b}III | R⁷VII |
| R³I | R^{a}VI | R^{b}III | R⁷VIII |
| R³I | R^{a}VI | R^{b}III | R⁷IX |
| R³I | R^{a}VI | R^{b}IV | — |
| R³I | R^{a}VI | R^{b}V | — |
| R³I | R^{a}VI | R^{b}VI | — |
| R³I | R^{a}VII | R^{b}I | R⁷I |
| R³I | R^{a}VII | R^{b}I | R⁷II |
| R³I | R^{a}VII | R^{b}I | R⁷III |
| R³I | R^{a}VII | R^{b}I | R⁷IV |
| R³I | R^{a}VII | R^{b}I | R⁷V |
| R³I | R^{a}VII | R^{b}I | R⁷VI |
| R³I | R^{a}VII | R^{b}I | R⁷VII |
| R³I | R^{a}VII | R^{b}I | R⁷VIII |
| R³I | R^{a}VII | R^{b}I | R⁷IX |
| R³I | R^{a}VII | R^{b}II | R⁷I |
| R³I | R^{a}VII | R^{b}II | R⁷II |
| R³I | R^{a}VII | R^{b}II | R⁷III |
| R³I | R^{a}VII | R^{b}II | R⁷IV |
| R³I | R^{a}VII | R^{b}II | R⁷V |
| R³I | R^{a}VII | R^{b}II | R⁷VI |
| R³I | R^{a}VII | R^{b}II | R⁷VII |
| R³I | R^{a}VII | R^{b}II | R⁷VIII |
| R³I | R^{a}VII | R^{b}II | R⁷IX |
| R³I | R^{a}VII | R^{b}III | R⁷I |
| R³I | R^{a}VII | R^{b}III | R⁷II |
| R³I | R^{a}VII | R^{b}III | R⁷III |
| R³I | R^{a}VII | R^{b}III | R⁷IV |
| R³I | R^{a}VII | R^{b}III | R⁷V |
| R³I | R^{a}VII | R^{b}III | R⁷VI |
| R³I | R^{a}VII | R^{b}III | R⁷VII |
| R³I | R^{a}VII | R^{b}III | R⁷VIII |
| R³I | R^{a}VII | R^{b}III | R⁷IX |
| R³I | R^{a}VII | R^{b}IV | — |
| R³I | R^{a}VII | R^{b}V | — |
| R³I | R^{a}VII | R^{b}VI | — |
| R³I | R^{a}VIII | R^{b}I | R⁷I |
| R³I | R^{a}VIII | R^{b}I | R⁷II |
| R³I | R^{a}VIII | R^{b}I | R⁷III |
| R³I | R^{a}VIII | R^{b}I | R⁷IV |
| R³I | R^{a}VIII | R^{b}I | R⁷V |
| R³I | R^{a}VIII | R^{b}I | R⁷VI |
| R³I | R^{a}VIII | R^{b}I | R⁷VII |
| R³I | R^{a}VIII | R^{b}I | R⁷VIII |
| R³I | R^{a}VIII | R^{b}I | R⁷IX |
| R³I | R^{a}VIII | R^{b}II | R⁷I |
| R³I | R^{a}VIII | R^{b}II | R⁷II |
| R³I | R^{a}VIII | R^{b}II | R⁷III |
| R³I | R^{a}VIII | R^{b}II | R⁷IV |
| R³I | R^{a}VIII | R^{b}II | R⁷V |
| R³I | R^{a}VIII | R^{b}II | R⁷VI |
| R³I | R^{a}VIII | R^{b}II | R⁷VII |
| R³I | R^{a}VIII | R^{b}II | R⁷VIII |
| R³I | R^{a}VIII | R^{b}II | R⁷IX |
| R³I | R^{a}VIII | R^{b}III | R⁷I |
| R³I | R^{a}VIII | R^{b}III | R⁷II |
| R³I | R^{a}VIII | R^{b}III | R⁷III |
| R³I | R^{a}VIII | R^{b}III | R⁷IV |
| R³I | R^{a}VIII | R^{b}III | R⁷V |
| R³I | R^{a}VIII | R^{b}III | R⁷VI |
| R³I | R^{a}VIII | R^{b}III | R⁷VII |
| R³I | R^{a}VIII | R^{b}III | R⁷VIII |
| R³I | R^{a}VIII | R^{b}III | R⁷IX |
| R³I | R^{a}VIII | R^{b}IV | — |
| R³I | R^{a}VIII | R^{b}V | — |
| R³I | R^{a}VIII | R^{b}VI | — |
| R³I | R^{a}IX | R^{b}I | R⁷I |
| R³I | R^{a}IX | R^{b}I | R⁷II |
| R³I | R^{a}IX | R^{b}I | R⁷III |
| R³I | R^{a}IX | R^{b}I | R⁷IV |
| R³I | R^{a}IX | R^{b}I | R⁷V |
| R³I | R^{a}IX | R^{b}I | R⁷VI |
| R³I | R^{a}IX | R^{b}I | R⁷VII |
| R³I | R^{a}IX | R^{b}I | R⁷VIII |
| R³I | R^{a}IX | R^{b}I | R⁷IX |
| R³I | R^{a}IX | R^{b}II | R⁷I |
| R³I | R^{a}IX | R^{b}II | R⁷II |
| R³I | R^{a}IX | R^{b}II | R⁷III |
| R³I | R^{a}IX | R^{b}II | R⁷IV |
| R³I | R^{a}IX | R^{b}II | R⁷V |
| R³I | R^{a}IX | R^{b}II | R⁷VI |
| R³I | R^{a}IX | R^{b}II | R⁷VII |
| R³I | R^{a}IX | R^{b}II | R⁷VIII |
| R³I | R^{a}IX | R^{b}II | R⁷IX |
| R³I | R^{a}IX | R^{b}III | R⁷I |
| R³I | R^{a}IX | R^{b}III | R⁷II |
| R³I | R^{a}IX | R^{b}III | R⁷III |
| R³I | R^{a}IX | R^{b}III | R⁷IV |
| R³I | R^{a}IX | R^{b}III | R⁷V |
| R³I | R^{a}IX | R^{b}III | R⁷VI |
| R³I | R^{a}IX | R^{b}III | R⁷VII |
| R³I | R^{a}IX | R^{b}III | R⁷VIII |
| R³I | R^{a}IX | R^{b}III | R⁷IX |
| R³I | R^{a}IX | R^{b}IV | — |
| R³I | R^{a}IX | R^{b}V | — |
| R³I | R^{a}IX | R^{b}VI | — |
| R³I | R^{a}X | R^{b}I | R⁷I |
| R³I | R^{a}X | R^{b}I | R⁷II |
| R³I | R^{a}X | R^{b}I | R⁷III |
| R³I | R^{a}X | R^{b}I | R⁷IV |
| R³I | R^{a}X | R^{b}I | R⁷V |
| R³I | R^{a}X | R^{b}I | R⁷VI |
| R³I | R^{a}X | R^{b}I | R⁷VII |
| R³I | R^{a}X | R^{b}I | R⁷VIII |
| R³I | R^{a}X | R^{b}I | R⁷IX |
| R³I | R^{a}X | R^{b}II | R⁷I |
| R³I | R^{a}X | R^{b}II | R⁷II |
| R³I | R^{a}X | R^{b}II | R⁷III |
| R³I | R^{a}X | R^{b}II | R⁷IV |
| R³I | R^{a}X | R^{b}II | R⁷V |
| R³I | R^{a}X | R^{b}II | R⁷VI |
| R³I | R^{a}X | R^{b}II | R⁷VII |
| R³I | R^{a}X | R^{b}II | R⁷VIII |
| R³I | R^{a}X | R^{b}II | R⁷IX |
| R³I | R^{a}X | R^{b}III | R⁷I |
| R³I | R^{a}X | R^{b}III | R⁷II |
| R³I | R^{a}X | R^{b}III | R⁷III |
| R³I | R^{a}X | R^{b}III | R⁷IV |
| R³I | R^{a}X | R^{b}III | R⁷V |
| R³I | R^{a}X | R^{b}III | R⁷VI |
| R³I | R^{a}X | R^{b}III | R⁷VII |
| R³I | R^{a}X | R^{b}III | R⁷VIII |
| R³I | R^{a}X | R^{b}III | R⁷IX |
| R³I | R^{a}X | R^{b}IV | — |
| R³I | R^{a}X | R^{b}V | — |
| R³I | R^{a}X | R^{b}VI | — |
| R³II | R^{a}I | R^{b}I | R⁷I |
| R³II | R^{a}I | R^{b}I | R⁷II |
| R³II | R^{a}I | R^{b}I | R⁷III |
| R³II | R^{a}I | R^{b}I | R⁷IV |
| R³II | R^{a}I | R^{b}I | R⁷V |
| R³II | R^{a}I | R^{b}I | R⁷VI |
| R³II | R^{a}I | R^{b}I | R⁷VII |
| R³II | R^{a}I | R^{b}I | R⁷VIII |
| R³II | R^{a}I | R^{b}I | R⁷IX |
| R³II | R^{a}I | R^{b}II | R⁷I |
| R³II | R^{a}I | R^{b}II | R⁷II |
| R³II | R^{a}I | R^{b}II | R⁷III |
| R³II | R^{a}I | R^{b}II | R⁷IV |
| R³II | R^{a}I | R^{b}II | R⁷V |
| R³II | R^{a}I | R^{b}II | R⁷VI |
| R³II | R^{a}I | R^{b}II | R⁷VII |
| R³II | R^{a}I | R^{b}II | R⁷VIII |
| R³II | R^{a}I | R^{b}II | R⁷IX |
| R³II | R^{a}I | R^{b}III | R⁷I |
| R³II | R^{a}I | R^{b}III | R⁷II |
| R³II | R^{a}I | R^{b}III | R⁷III |
| R³II | R^{a}I | R^{b}III | R⁷IV |
| R³II | R^{a}I | R^{b}III | R⁷V |
| R³II | R^{a}I | R^{b}III | R⁷VI |
| R³II | R^{a}I | R^{b}III | R⁷VII |
| R³II | R^{a}I | R^{b}III | R⁷VIII |
| R³II | R^{a}I | R^{b}III | R⁷IX |
| R³II | R^{a}I | R^{b}IV | — |
| R³II | R^{a}I | R^{b}V | — |
| R³II | R^{a}I | R^{b}VI | — |
| R³II | R^{a}II | R^{b}I | R⁷I |
| R³II | R^{a}II | R^{b}I | R⁷II |
| R³II | R^{a}II | R^{b}I | R⁷III |
| R³II | R^{a}II | R^{b}I | R⁷IV |
| R³II | R^{a}II | R^{b}I | R⁷V |
| R³II | R^{a}II | R^{b}I | R⁷VI |
| R³II | R^{a}II | R^{b}I | R⁷VII |
| R³II | R^{a}II | R^{b}I | R⁷VIII |
| R³II | R^{a}II | R^{b}I | R⁷IX |
| R³II | R^{a}II | R^{b}II | R⁷I |
| R³II | R^{a}II | R^{b}II | R⁷II |
| R³II | R^{a}II | R^{b}II | R⁷III |
| R³II | R^{a}II | R^{b}II | R⁷IV |
| R³II | R^{a}II | R^{b}II | R⁷V |
| R³II | R^{a}II | R^{b}II | R⁷VI |
| R³II | R^{a}II | R^{b}II | R⁷VII |
| R³II | R^{a}II | R^{b}II | R⁷VIII |
| R³II | R^{a}II | R^{b}II | R⁷IX |
| R³II | R^{a}II | R^{b}III | R⁷I |
| R³II | R^{a}II | R^{b}III | R⁷II |
| R³II | R^{a}II | R^{b}III | R⁷III |
| R³II | R^{a}II | R^{b}III | R⁷IV |
| R³II | R^{a}II | R^{b}III | R⁷V |
| R³II | R^{a}II | R^{b}III | R⁷VI |
| R³II | R^{a}II | R^{b}III | R⁷VII |
| R³II | R^{a}II | R^{b}III | R⁷VIII |
| R³II | R^{a}II | R^{b}III | R⁷IX |
| R³II | R^{a}II | R^{b}IV | — |
| R³II | R^{a}II | R^{b}V | — |
| R³II | R^{a}II | R^{b}VI | — |
| R³II | R^{a}III | R^{b}I | R⁷I |
| R³II | R^{a}III | R^{b}I | R⁷II |
| R³II | R^{a}III | R^{b}I | R⁷III |
| R³II | R^{a}III | R^{b}I | R⁷IV |
| R³II | R^{a}III | R^{b}I | R⁷V |
| R³II | R^{a}III | R^{b}I | R⁷VI |
| R³II | R^{a}III | R^{b}I | R⁷VII |
| R³II | R^{a}III | R^{b}I | R⁷VIII |
| R³II | R^{a}III | R^{b}I | R⁷IX |
| R³II | R^{a}III | R^{b}II | R⁷I |
| R³II | R^{a}III | R^{b}II | R⁷II |
| R³II | R^{a}III | R^{b}II | R⁷III |
| R³II | R^{a}III | R^{b}II | R⁷IV |
| R³II | R^{a}III | R^{b}II | R⁷V |
| R³II | R^{a}III | R^{b}II | R⁷VI |
| R³II | R^{a}III | R^{b}II | R⁷VII |
| R³II | R^{a}III | R^{b}II | R⁷VIII |
| R³II | R^{a}III | R^{b}II | R⁷IX |
| R³II | R^{a}III | R^{b}III | R⁷I |
| R³II | R^{a}III | R^{b}III | R⁷II |
| R³II | R^{a}III | R^{b}III | R⁷III |
| R³II | R^{a}III | R^{b}III | R⁷IV |
| R³II | R^{a}III | R^{b}III | R⁷V |
| R³II | R^{a}III | R^{b}III | R⁷VI |
| R³II | R^{a}III | R^{b}III | R⁷VII |
| R³II | R^{a}III | R^{b}III | R⁷VIII |
| R³II | R^{a}III | R^{b}III | R⁷IX |
| R³II | R^{a}III | R^{b}IV | — |
| R³II | R^{a}III | R^{b}V | — |
| R³II | R^{a}III | R^{b}VI | — |
| R³II | R^{a}IV | R^{b}I | R⁷I |
| R³II | R^{a}IV | R^{b}I | R⁷II |
| R³II | R^{a}IV | R^{b}I | R⁷III |
| R³II | R^{a}IV | R^{b}I | R⁷IV |
| R³II | R^{a}IV | R^{b}I | R⁷V |
| R³II | R^{a}IV | R^{b}I | R⁷VI |
| R³II | R^{a}IV | R^{b}I | R⁷VII |
| R³II | R^{a}IV | R^{b}I | R⁷VIII |
| R³II | R^{a}IV | R^{b}I | R⁷IX |
| R³II | R^{a}IV | R^{b}II | R⁷I |
| R³II | R^{a}IV | R^{b}II | R⁷II |
| R³II | R^{a}IV | R^{b}II | R⁷III |
| R³II | R^{a}IV | R^{b}II | R⁷IV |
| R³II | R^{a}IV | R^{b}II | R⁷V |
| R³II | R^{a}IV | R^{b}II | R⁷VI |
| R³II | R^{a}IV | R^{b}II | R⁷VII |
| R³II | R^{a}IV | R^{b}II | R⁷VIII |
| R³II | R^{a}IV | R^{b}II | R⁷IX |
| R³II | R^{a}IV | R^{b}III | R⁷I |
| R³II | R^{a}IV | R^{b}III | R⁷II |
| R³II | R^{a}IV | R^{b}III | R⁷III |
| R³II | R^{a}IV | R^{b}III | R⁷IV |
| R³II | R^{a}IV | R^{b}III | R⁷V |
| R³II | R^{a}IV | R^{b}III | R⁷VI |
| R³II | R^{a}IV | R^{b}III | R⁷VII |
| R³II | R^{a}IV | R^{b}III | R⁷VIII |
| R³II | R^{a}IV | R^{b}III | R⁷IX |
| R³II | R^{a}IV | R^{b}IV | — |
| R³II | R^{a}IV | R^{b}V | — |
| R³II | R^{a}IV | R^{b}VI | — |
| R³II | R^{a}V | R^{b}I | R⁷I |
| R³II | R^{a}V | R^{b}I | R⁷II |
| R³II | R^{a}V | R^{b}I | R⁷III |
| R³II | R^{a}V | R^{b}I | R⁷IV |
| R³II | R^{a}V | R^{b}I | R⁷V |
| R³II | R^{a}V | R^{b}I | R⁷VI |
| R³II | R^{a}V | R^{b}I | R⁷VII |
| R³II | R^{a}V | R^{b}I | R⁷VIII |
| R³II | R^{a}V | R^{b}I | R⁷IX |
| R³II | R^{a}V | R^{b}II | R⁷I |
| R³II | R^{a}V | R^{b}II | R⁷II |
| R³II | R^{a}V | R^{b}II | R⁷III |
| R³II | R^{a}V | R^{b}II | R⁷IV |
| R³II | R^{a}V | R^{b}II | R⁷V |
| R³II | R^{a}V | R^{b}II | R⁷VI |
| R³II | R^{a}V | R^{b}II | R⁷VII |
| R³II | R^{a}V | R^{b}II | R⁷VIII |
| R³II | R^{a}V | R^{b}II | R⁷IX |
| R³II | R^{a}V | R^{b}III | R⁷I |
| R³II | R^{a}V | R^{b}III | R⁷II |
| R³II | R^{a}V | R^{b}III | R⁷III |
| R³II | R^{a}V | R^{b}III | R⁷IV |
| R³II | R^{a}V | R^{b}III | R⁷V |
| R³II | R^{a}V | R^{b}III | R⁷VI |
| R³II | R^{a}V | R^{b}III | R⁷VII |
| R³II | R^{a}V | R^{b}III | R⁷VIII |
| R³II | R^{a}V | R^{b}III | R⁷IX |
| R³II | R^{a}V | R^{b}IV | — |
| R³II | R^{a}V | R^{b}V | — |
| R³II | R^{a}V | R^{b}VI | — |
| R³II | R^{a}VI | R^{b}I | R⁷I |
| R³II | R^{a}VI | R^{b}I | R⁷II |
| R³II | R^{a}VI | R^{b}I | R⁷III |
| R³II | R^{a}VI | R^{b}I | R⁷IV |
| R³II | R^{a}VI | R^{b}I | R⁷V |
| R³II | R^{a}VI | R^{b}I | R⁷VI |
| R³II | R^{a}VI | R^{b}I | R⁷VII |
| R³II | R^{a}VI | R^{b}I | R⁷VIII |
| R³II | R^{a}VI | R^{b}I | R⁷IX |
| R³II | R^{a}VI | R^{b}II | R⁷I |
| R³II | R^{a}VI | R^{b}II | R⁷II |
| R³II | R^{a}VI | R^{b}II | R⁷III |
| R³II | R^{a}VI | R^{b}II | R⁷IV |
| R³II | R^{a}VI | R^{b}II | R⁷V |
| R³II | R^{a}VI | R^{b}II | R⁷VI |
| R³II | R^{a}VI | R^{b}II | R⁷VII |
| R³II | R^{a}VI | R^{b}II | R⁷VIII |
| R³II | R^{a}VI | R^{b}II | R⁷IX |
| R³II | R^{a}VI | R^{b}III | R⁷I |
| R³II | R^{a}VI | R^{b}III | R⁷II |
| R³II | R^{a}VI | R^{b}III | R⁷III |
| R³II | R^{a}VI | R^{b}III | R⁷IV |
| R³II | R^{a}VI | R^{b}III | R⁷V |
| R³II | R^{a}VI | R^{b}III | R⁷VI |
| R³II | R^{a}VI | R^{b}III | R⁷VII |
| R³II | R^{a}VI | R^{b}III | R⁷VIII |
| R³II | R^{a}VI | R^{b}III | R⁷IX |
| R³II | R^{a}VI | R^{b}IV | — |
| R³II | R^{a}VI | R^{b}V | — |
| R³II | R^{a}VI | R^{b}VI | — |
| R³II | R^{a}VII | R^{b}I | R⁷I |
| R³II | R^{a}VII | R^{b}I | R⁷II |
| R³II | R^{a}VII | R^{b}I | R⁷III |
| R³II | R^{a}VII | R^{b}I | R⁷IV |
| R³II | R^{a}VII | R^{b}I | R⁷V |
| R³II | R^{a}VII | R^{b}I | R⁷VI |
| R³II | R^{a}VII | R^{b}I | R⁷VII |
| R³II | R^{a}VII | R^{b}I | R⁷VIII |
| R³II | R^{a}VII | R^{b}I | R⁷IX |
| R³II | R^{a}VII | R^{b}II | R⁷I |
| R³II | R^{a}VII | R^{b}II | R⁷II |
| R³II | R^{a}VII | R^{b}II | R⁷III |
| R³II | R^{a}VII | R^{b}II | R⁷IV |
| R³II | R^{a}VII | R^{b}II | R⁷V |
| R³II | R^{a}VII | R^{b}II | R⁷VI |
| R³II | R^{a}VII | R^{b}II | R⁷VII |
| R³II | R^{a}VII | R^{b}II | R⁷VIII |
| R³II | R^{a}VII | R^{b}II | R⁷IX |
| R³II | R^{a}VII | R^{b}III | R⁷I |
| R³II | R^{a}VII | R^{b}III | R⁷II |
| R³II | R^{a}VII | R^{b}III | R⁷III |
| R³II | R^{a}VII | R^{b}III | R⁷IV |
| R³II | R^{a}VII | R^{b}III | R⁷V |
| R³II | R^{a}VII | R^{b}III | R⁷VI |
| R³II | R^{a}VII | R^{b}III | R⁷VII |
| R³II | R^{a}VII | R^{b}III | R⁷VIII |
| R³II | R^{a}VII | R^{b}III | R⁷IX |
| R³II | R^{a}VII | R^{b}IV | — |
| R³II | R^{a}VII | R^{b}V | — |
| R³II | R^{a}VII | R^{b}VI | — |
| R³II | R^{a}VIII | R^{b}I | R⁷I |
| R³II | R^{a}VIII | R^{b}I | R⁷II |
| R³II | R^{a}VIII | R^{b}I | R⁷III |
| R³II | R^{a}VIII | R^{b}I | R⁷IV |
| R³II | R^{a}VIII | R^{b}I | R⁷V |
| R³II | R^{a}VIII | R^{b}I | R⁷VI |
| R³II | R^{a}VIII | R^{b}I | R⁷VII |
| R³II | R^{a}VIII | R^{b}I | R⁷VIII |
| R³II | R^{a}VIII | R^{b}I | R⁷IX |
| R³II | R^{a}VIII | R^{b}II | R⁷I |
| R³II | R^{a}VIII | R^{b}II | R⁷II |
| R³II | R^{a}VIII | R^{b}II | R⁷III |
| R³II | R^{a}VIII | R^{b}II | R⁷IV |
| R³II | R^{a}VIII | R^{b}II | R⁷V |
| R³II | R^{a}VIII | R^{b}II | R⁷VI |
| R³II | R^{a}VIII | R^{b}II | R⁷VII |
| R³II | R^{a}VIII | R^{b}II | R⁷VIII |
| R³II | R^{a}VIII | R^{b}II | R⁷IX |
| R³II | R^{a}VIII | R^{b}III | R⁷I |
| R³II | R^{a}VIII | R^{b}III | R⁷II |
| R³II | R^{a}VIII | R^{b}III | R⁷III |
| R³II | R^{a}VIII | R^{b}III | R⁷IV |
| R³II | R^{a}VIII | R^{b}III | R⁷V |
| R³II | R^{a}VIII | R^{b}III | R⁷VI |
| R³II | R^{a}VIII | R^{b}III | R⁷VII |
| R³II | R^{a}VIII | R^{b}III | R⁷VIII |
| R³II | R^{a}VIII | R^{b}III | R⁷IX |
| R³II | R^{a}VIII | R^{b}IV | — |
| R³II | R^{a}VIII | R^{b}V | — |
| R³II | R^{a}VIII | R^{b}VI | — |
| R³II | R^{a}IX | R^{b}I | R⁷I |
| R³II | R^{a}IX | R^{b}I | R⁷II |
| R³II | R^{a}IX | R^{b}I | R⁷III |
| R³II | R^{a}IX | R^{b}I | R⁷IV |
| R³II | R^{a}IX | R^{b}I | R⁷V |
| R³II | R^{a}IX | R^{b}I | R⁷VI |
| R³II | R^{a}IX | R^{b}I | R⁷VII |
| R³II | R^{a}IX | R^{b}I | R⁷VIII |
| R³II | R^{a}IX | R^{b}I | R⁷IX |
| R³II | R^{a}IX | R^{b}II | R⁷I |
| R³II | R^{a}IX | R^{b}II | R⁷II |
| R³II | R^{a}IX | R^{b}II | R⁷III |
| R³II | R^{a}IX | R^{b}II | R⁷IV |
| R³II | R^{a}IX | R^{b}II | R⁷V |
| R³II | R^{a}IX | R^{b}II | R⁷VI |
| R³II | R^{a}IX | R^{b}II | R⁷VII |
| R³II | R^{a}IX | R^{b}II | R⁷VIII |
| R³II | R^{a}IX | R^{b}II | R⁷IX |
| R³II | R^{a}IX | R^{b}III | R⁷I |
| R³II | R^{a}IX | R^{b}III | R⁷II |
| R³II | R^{a}IX | R^{b}III | R⁷III |
| R³II | R^{a}IX | R^{b}III | R⁷IV |
| R³II | R^{a}IX | R^{b}III | R⁷V |
| R³II | R^{a}IX | R^{b}III | R⁷VI |
| R³II | R^{a}IX | R^{b}III | R⁷VII |
| R³II | R^{a}IX | R^{b}III | R⁷VIII |
| R³II | R^{a}IX | R^{b}III | R⁷IX |
| R³II | R^{a}IX | R^{b}IV | — |
| R³II | R^{a}IX | R^{b}V | — |
| R³II | R^{a}IX | R^{b}VI | — |
| R³II | R^{a}X | R^{b}I | R⁷I |
| R³II | R^{a}X | R^{b}I | R⁷II |
| R³II | R^{a}X | R^{b}I | R⁷III |
| R³II | R^{a}X | R^{b}I | R⁷IV |
| R³II | R^{a}X | R^{b}I | R⁷V |
| R³II | R^{a}X | R^{b}I | R⁷VI |
| R³II | R^{a}X | R^{b}I | R⁷VII |
| R³II | R^{a}X | R^{b}I | R⁷VIII |
| R³II | R^{a}X | R^{b}I | R⁷IX |
| R³II | R^{a}X | R^{b}II | R⁷I |
| R³II | R^{a}X | R^{b}II | R⁷II |
| R³II | R^{a}X | R^{b}II | R⁷III |
| R³II | R^{a}X | R^{b}II | R⁷IV |
| R³II | R^{a}X | R^{b}II | R⁷V |
| R³II | R^{a}X | R^{b}II | R⁷VI |
| R³II | R^{a}X | R^{b}II | R⁷VII |
| R³II | R^{a}X | R^{b}II | R⁷VIII |
| R³II | R^{a}X | R^{b}II | R⁷IX |
| R³II | R^{a}X | R^{b}III | R⁷I |
| R³II | R^{a}X | R^{b}III | R⁷II |
| R³II | R^{a}X | R^{b}III | R⁷III |
| R³II | R^{a}X | R^{b}III | R⁷IV |
| R³II | R^{a}X | R^{b}III | R⁷V |
| R³II | R^{a}X | R^{b}III | R⁷VI |
| R³II | R^{a}X | R^{b}III | R⁷VII |
| R³II | R^{a}X | R^{b}III | R⁷VIII |
| R³II | R^{a}X | R^{b}III | R⁷IX |
| R³II | R^{a}X | R^{b}IV | — |
| R³II | R^{a}X | R^{b}V | — |
| R³II | R^{a}X | R^{b}VI | — |
| R³III | R^{a}I | R^{b}I | R⁷I |
| R³III | R^{a}I | R^{b}I | R⁷II |
| R³III | R^{a}I | R^{b}I | R⁷III |
| R³III | R^{a}I | R^{b}I | R⁷IV |
| R³III | R^{a}I | R^{b}I | R⁷V |
| R³III | R^{a}I | R^{b}I | R⁷VI |
| R³III | R^{a}I | R^{b}I | R⁷VII |
| R³III | R^{a}I | R^{b}I | R⁷VIII |
| R³III | R^{a}I | R^{b}I | R⁷IX |
| R³III | R^{a}I | R^{b}II | R⁷I |
| R³III | R^{a}I | R^{b}II | R⁷II |
| R³III | R^{a}I | R^{b}II | R⁷III |
| R³III | R^{a}I | R^{b}II | R⁷IV |
| R³III | R^{a}I | R^{b}II | R⁷V |
| R³III | R^{a}I | R^{b}II | R⁷VI |
| R³III | R^{a}I | R^{b}II | R⁷VII |
| R³III | R^{a}I | R^{b}II | R⁷VIII |
| R³III | R^{a}I | R^{b}II | R⁷IX |
| R³III | R^{a}I | R^{b}III | R⁷I |
| R³III | R^{a}I | R^{b}III | R⁷II |
| R³III | R^{a}I | R^{b}III | R⁷III |
| R³III | R^{a}I | R^{b}III | R⁷IV |
| R³III | R^{a}I | R^{b}III | R⁷V |
| R³III | R^{a}I | R^{b}III | R⁷VI |
| R³III | R^{a}I | R^{b}III | R⁷VII |
| R³III | R^{a}I | R^{b}III | R⁷VIII |
| R³III | R^{a}I | R^{b}III | R⁷IX |
| R³III | R^{a}I | R^{b}IV | — |
| R³III | R^{a}I | R^{b}V | — |
| R³III | R^{a}I | R^{b}VI | — |
| R³III | R^{a}II | R^{b}I | R⁷I |
| R³III | R^{a}II | R^{b}I | R⁷II |
| R³III | R^{a}II | R^{b}I | R⁷III |
| R³III | R^{a}II | R^{b}I | R⁷IV |
| R³III | R^{a}II | R^{b}I | R⁷V |
| R³III | R^{a}II | R^{b}I | R⁷VI |
| R³III | R^{a}II | R^{b}I | R⁷VII |
| R³III | R^{a}II | R^{b}I | R⁷VIII |
| R³III | R^{a}II | R^{b}I | R⁷IX |
| R³III | R^{a}II | R^{b}II | R⁷I |
| R³III | R^{a}II | R^{b}II | R⁷II |
| R³III | R^{a}II | R^{b}II | R⁷III |
| R³III | R^{a}II | R^{b}II | R⁷IV |
| R³III | R^{a}II | R^{b}II | R⁷V |
| R³III | R^{a}II | R^{b}II | R⁷VI |
| R³III | R^{a}II | R^{b}II | R⁷VII |
| R³III | R^{a}II | R^{b}II | R⁷VIII |
| R³III | R^{a}II | R^{b}II | R⁷IX |
| R³III | R^{a}II | R^{b}III | R⁷I |
| R³III | R^{a}II | R^{b}III | R⁷II |
| R³III | R^{a}II | R^{b}III | R⁷III |
| R³III | R^{a}II | R^{b}III | R⁷IV |
| R³III | R^{a}II | R^{b}III | R⁷V |
| R³III | R^{a}II | R^{b}III | R⁷VI |
| R³III | R^{a}II | R^{b}III | R⁷VII |
| R³III | R^{a}II | R^{b}III | R⁷VIII |
| R³III | R^{a}II | R^{b}III | R⁷IX |
| R³III | R^{a}II | R^{b}IV | — |
| R³III | R^{a}II | R^{b}V | — |
| R³III | R^{a}II | R^{b}VI | — |
| R³III | R^{a}III | R^{b}I | R⁷I |
| R³III | R^{a}III | R^{b}I | R⁷II |
| R³III | R^{a}III | R^{b}I | R⁷III |
| R³III | R^{a}III | R^{b}I | R⁷IV |
| R³III | R^{a}III | R^{b}I | R⁷V |
| R³III | R^{a}III | R^{b}I | R⁷VI |
| R³III | R^{a}III | R^{b}I | R⁷VII |
| R³III | R^{a}III | R^{b}I | R⁷VIII |
| R³III | R^{a}III | R^{b}I | R⁷IX |
| R³III | R^{a}III | R^{b}II | R⁷I |
| R³III | R^{a}III | R^{b}II | R⁷II |
| R³III | R^{a}III | R^{b}II | R⁷III |
| R³III | R^{a}III | R^{b}II | R⁷IV |
| R³III | R^{a}III | R^{b}II | R⁷V |
| R³III | R^{a}III | R^{b}II | R⁷VI |
| R³III | R^{a}III | R^{b}II | R⁷VII |
| R³III | R^{a}III | R^{b}II | R⁷VIII |
| R³III | R^{a}III | R^{b}II | R⁷IX |
| R³III | R^{a}III | R^{b}III | R⁷I |
| R³III | R^{a}III | R^{b}III | R⁷II |
| R³III | R^{a}III | R^{b}III | R⁷III |
| R³III | R^{a}III | R^{b}III | R⁷IV |
| R³III | R^{a}III | R^{b}III | R⁷V |
| R³III | R^{a}III | R^{b}III | R⁷VI |
| R³III | R^{a}III | R^{b}III | R⁷VII |
| R³III | R^{a}III | R^{b}III | R⁷VIII |
| R³III | R^{a}III | R^{b}III | R⁷IX |
| R³III | R^{a}III | R^{b}IV | — |
| R³III | R^{a}III | R^{b}V | — |
| R³III | R^{a}III | R^{b}VI | — |
| R³III | R^{a}IV | R^{b}I | R⁷I |
| R³III | R^{a}IV | R^{b}I | R⁷II |
| R³III | R^{a}IV | R^{b}I | R⁷III |
| R³III | R^{a}IV | R^{b}I | R⁷IV |
| R³III | R^{a}IV | R^{b}I | R⁷V |
| R³III | R^{a}IV | R^{b}I | R⁷VI |
| R³III | R^{a}IV | R^{b}I | R⁷VII |
| R³III | R^{a}IV | R^{b}I | R⁷VIII |
| R³III | R^{a}IV | R^{b}I | R⁷IX |
| R³III | R^{a}IV | R^{b}II | R⁷I |
| R³III | R^{a}IV | R^{b}II | R⁷II |
| R³III | R^{a}IV | R^{b}II | R⁷IV |
| R³III | R^{a}IV | R^{b}II | R⁷V |
| R³III | R^{a}IV | R^{b}II | R⁷VI |
| R³III | R^{a}IV | R^{b}II | R⁷III |
| R³III | R^{a}IV | R^{b}II | R⁷VII |
| R³III | R^{a}IV | R^{b}II | R⁷VIII |
| R³III | R^{a}IV | R^{b}II | R⁷IX |
| R³III | R^{a}IV | R^{b}III | R⁷I |
| R³III | R^{a}IV | R^{b}III | R⁷II |
| R³III | R^{a}IV | R^{b}III | R⁷III |
| R³III | R^{a}IV | R^{b}III | R⁷IV |
| R³III | R^{a}IV | R^{b}III | R⁷V |
| R³III | R^{a}IV | R^{b}III | R⁷VI |
| R³III | R^{a}IV | R^{b}III | R⁷VII |
| R³III | R^{a}IV | R^{b}III | R⁷VIII |
| R³III | R^{a}IV | R^{b}III | R⁷IX |
| R³III | R^{a}IV | R^{b}IV | — |
| R³III | R^{a}IV | R^{b}V | — |
| R³III | R^{a}IV | R^{b}VI | — |
| R³III | R^{a}V | R^{b}I | R⁷I |
| R³III | R^{a}V | R^{b}I | R⁷II |
| R³III | R^{a}V | R^{b}I | R⁷III |
| R³III | R^{a}V | R^{b}I | R⁷IV |
| R³III | R^{a}V | R^{b}I | R⁷V |
| R³III | R^{a}V | R^{b}I | R⁷VI |
| R³III | R^{a}V | R^{b}I | R⁷VII |
| R³III | R^{a}V | R^{b}I | R⁷VIII |
| R³III | R^{a}V | R^{b}I | R⁷IX |
| R³III | R^{a}V | R^{b}II | R⁷I |
| R³III | R^{a}V | R^{b}II | R⁷II |
| R³III | R^{a}V | R^{b}II | R⁷III |
| R³III | R^{a}V | R^{b}II | R⁷IV |
| R³III | R^{a}V | R^{b}II | R⁷V |
| R³III | R^{a}V | R^{b}II | R⁷VI |
| R³III | R^{a}V | R^{b}II | R⁷VII |
| R³III | R^{a}V | R^{b}II | R⁷VIII |
| R³III | R^{a}V | R^{b}II | R⁷IX |
| R³III | R^{a}V | R^{b}III | R⁷I |
| R³III | R^{a}V | R^{b}III | R⁷II |
| R³III | R^{a}V | R^{b}III | R⁷III |
| R³III | R^{a}V | R^{b}III | R⁷IV |
| R³III | R^{a}V | R^{b}III | R⁷V |
| R³III | R^{a}V | R^{b}III | R⁷VI |
| R³III | R^{a}V | R^{b}III | R⁷VII |
| R³III | R^{a}V | R^{b}III | R⁷VIII |
| R³III | R^{a}V | R^{b}III | R⁷IX |
| R³III | R^{a}V | R^{b}IV | — |
| R³III | R^{a}V | R^{b}V | — |
| R³III | R^{a}V | R^{b}VI | — |
| R³III | R^{a}VI | R^{b}I | R⁷I |
| R³III | R^{a}VI | R^{b}I | R⁷II |
| R³III | R^{a}VI | R^{b}I | R⁷III |
| R³III | R^{a}VI | R^{b}I | R⁷IV |
| R³III | R^{a}VI | R^{b}I | R⁷V |
| R³III | R^{a}VI | R^{b}I | R⁷VI |
| R³III | R^{a}VI | R^{b}I | R⁷VII |
| R³III | R^{a}VI | R^{b}I | R⁷VIII |
| R³III | R^{a}VI | R^{b}I | R⁷IX |
| R³III | R^{a}VI | R^{b}II | R⁷I |
| R³III | R^{a}VI | R^{b}II | R⁷II |
| R³III | R^{a}VI | R^{b}II | R⁷III |
| R³III | R^{a}VI | R^{b}II | R⁷IV |
| R³III | R^{a}VI | R^{b}II | R⁷V |
| R³III | R^{a}VI | R^{b}II | R⁷VI |
| R³III | R^{a}VI | R^{b}II | R⁷VII |
| R³III | R^{a}VI | R^{b}II | R⁷VIII |
| R³III | R^{a}VI | R^{b}II | R⁷IX |
| R³III | R^{a}VI | R^{b}III | R⁷I |
| R³III | R^{a}VI | R^{b}III | R⁷II |
| R³III | R^{a}VI | R^{b}III | R⁷III |
| R³III | R^{a}VI | R^{b}III | R⁷IV |
| R³III | R^{a}VI | R^{b}III | R⁷V |
| R³III | R^{a}VI | R^{b}III | R⁷VI |
| R³III | R^{a}VI | R^{b}III | R⁷VII |
| R³III | R^{a}VI | R^{b}III | R⁷VIII |
| R³III | R^{a}VI | R^{b}III | R⁷IX |
| R³III | R^{a}VI | R^{b}IV | — |
| R³III | R^{a}VI | R^{b}V | — |
| R³III | R^{a}VI | R^{b}VI | — |
| R³III | R^{a}VII | R^{b}I | R⁷I |
| R³III | R^{a}VII | R^{b}I | R⁷II |
| R³III | R^{a}VII | R^{b}I | R⁷III |
| R³III | R^{a}VII | R^{b}I | R⁷IV |
| R³III | R^{a}VII | R^{b}I | R⁷V |
| R³III | R^{a}VII | R^{b}I | R⁷VI |
| R³III | R^{a}VII | R^{b}I | R⁷VII |
| R³III | R^{a}VII | R^{b}I | R⁷VIII |
| R³III | R^{a}VII | R^{b}I | R⁷IX |
| R³III | R^{a}VII | R^{b}II | R⁷I |
| R³III | R^{a}VII | R^{b}II | R⁷II |
| R³III | R^{a}VII | R^{b}II | R⁷III |
| R³III | R^{a}VII | R^{b}II | R⁷IV |
| R³III | R^{a}VII | R^{b}II | R⁷V |
| R³III | R^{a}VII | R^{b}II | R⁷VI |
| R³III | R^{a}VII | R^{b}II | R^{a}VII |
| R³III | R^{a}VII | R^{b}II | R⁷VIII |
| R³III | R^{a}VII | R^{b}II | R⁷IX |
| R³III | R^{a}VII | R^{b}III | R⁷I |
| R³III | R^{a}VII | R^{b}III | R⁷II |
| R³III | R^{a}VII | R^{b}III | R⁷III |
| R³III | R^{a}VII | R^{b}III | R⁷IV |
| R³III | R^{a}VII | R^{b}III | R⁷V |
| R³III | R^{a}VII | R^{b}III | R⁷VI |
| R³III | R^{a}VII | R^{b}III | R⁷VII |
| R³III | R^{a}VII | R^{b}III | R⁷VIII |
| R³III | R^{a}VII | R^{b}III | R⁷IX |
| R³III | R^{a}VII | R^{b}IV | — |
| R³III | R^{a}VII | R^{b}V | — |
| R³III | R^{a}VII | R^{b}VI | — |
| R³III | R^{a}VIII | R^{b}I | R⁷I |
| R³III | R^{a}VIII | R^{b}I | R⁷II |
| R³III | R^{a}VIII | R^{b}I | R⁷III |
| R³III | R^{a}VIII | R^{b}I | R⁷IV |
| R³III | R^{a}VIII | R^{b}I | R⁷V |
| R³III | R^{a}VIII | R^{b}I | R⁷VI |
| R³III | R^{a}VIII | R^{b}I | R⁷VII |
| R³III | R^{a}VIII | R^{b}I | R⁷VIII |
| R³III | R^{a}VIII | R^{b}I | R⁷IX |
| R³III | R^{a}VIII | R^{b}II | R⁷I |
| R³III | R^{a}VIII | R^{b}II | R⁷II |
| R³III | R^{a}VIII | R^{b}II | R⁷III |
| R³III | R^{a}VIII | R^{b}II | R⁷IV |
| R³III | R^{a}VIII | R^{b}II | R⁷V |
| R³III | R^{a}VIII | R^{b}II | R⁷VI |
| R³III | R^{a}VIII | R^{b}II | R⁷VII |
| R³III | R^{a}VIII | R^{b}II | R⁷VIII |
| R³III | R^{a}VIII | R^{b}II | R⁷IX |
| R³III | R^{a}VIII | R^{b}III | R⁷I |
| R³III | R^{a}VIII | R^{b}III | R⁷II |
| R³III | R^{a}VIII | R^{b}III | R⁷III |
| R³III | R^{a}VIII | R^{b}III | R⁷IV |
| R³III | R^{a}VIII | R^{b}III | R⁷V |
| R³III | R^{a}VIII | R^{b}III | R⁷VI |
| R³III | R^{a}VIII | R^{b}III | R⁷VII |
| R³III | R^{a}VIII | R^{b}III | R⁷VIII |
| R³III | R^{a}VIII | R^{b}III | R⁷IX |
| R³III | R^{a}VIII | R^{b}IV | — |
| R³III | R^{a}VIII | R^{b}V | — |
| R³III | R^{a}VIII | R^{b}VI | — |
| R³III | R^{a}IX | R^{b}I | R⁷I |
| R³III | R^{a}IX | R^{b}I | R⁷II |
| R³III | R^{a}IX | R^{b}I | R⁷III |
| R³III | R^{a}IX | R^{b}I | R⁷IV |
| R³III | R^{a}IX | R^{b}I | R⁷V |
| R³III | R^{a}IX | R^{b}I | R⁷VI |
| R³III | R^{a}IX | R^{b}I | R⁷VII |
| R³III | R^{a}IX | R^{b}I | R⁷VIII |
| R³III | R^{a}IX | R^{b}I | R⁷IX |
| R³III | R^{a}IX | R^{b}II | R⁷I |
| R³III | R^{a}IX | R^{b}II | R⁷II |
| R³III | R^{a}IX | R^{b}II | R⁷III |
| R³III | R^{a}IX | R^{b}II | R⁷IV |
| R³III | R^{a}IX | R^{b}II | R⁷V |
| R³III | R^{a}IX | R^{b}II | R⁷VI |
| R³III | R^{a}IX | R^{b}II | R⁷VII |
| R³III | R^{a}IX | R^{b}II | R⁷VIII |
| R³III | R^{a}IX | R^{b}II | R⁷IX |
| R³III | R^{a}IX | R^{b}III | R⁷I |
| R³III | R^{a}IX | R^{b}III | R⁷II |
| R³III | R^{a}IX | R^{b}III | R⁷III |
| R³III | R^{a}IX | R^{b}III | R⁷IV |
| R³III | R^{a}IX | R^{b}III | R⁷V |
| R³III | R^{a}IX | R^{b}III | R⁷VI |
| R³III | R^{a}IX | R^{b}III | R⁷VII |
| R³III | R^{a}IX | R^{b}III | R⁷VIII |
| R³III | R^{a}IX | R^{b}III | R⁷IX |
| R³III | R^{a}IX | R^{b}IV | — |
| R³III | R^{a}IX | R^{b}V | — |
| R³III | R^{a}IX | R^{b}VI | — |
| R³III | R^{a}X | R^{b}I | R⁷I |
| R³III | R^{a}X | R^{b}I | R⁷II |
| R³III | R^{a}X | R^{b}I | R⁷III |
| R³III | R^{a}X | R^{b}I | R⁷IV |
| R³III | R^{a}X | R^{b}I | R⁷V |
| R³III | R^{a}X | R^{b}I | R⁷VI |
| R³III | R^{a}X | R^{b}I | R⁷VII |
| R³III | R^{a}X | R^{b}I | R⁷VIII |
| R³III | R^{a}X | R^{b}I | R⁷IX |
| R³III | R^{a}X | R^{b}II | R⁷I |
| R³III | R^{a}X | R^{b}II | R⁷II |
| R³III | R^{a}X | R^{b}II | R⁷III |
| R³III | R^{a}X | R^{b}II | R⁷IV |
| R³III | R^{a}X | R^{b}II | R⁷V |
| R³III | R^{a}X | R^{b}II | R⁷VI |
| R³III | R^{a}X | R^{b}II | R⁷VII |
| R³III | R^{a}X | R^{b}II | R⁷VIII |
| R³III | R^{a}X | R^{b}II | R⁷IX |
| R³III | R^{a}X | R^{b}III | R⁷I |
| R³III | R^{a}X | R^{b}III | R⁷II |
| R³III | R^{a}X | R^{b}III | R⁷III |
| R³III | R^{a}X | R^{b}III | R⁷IV |
| R³III | R^{a}X | R^{b}III | R⁷V |
| R³III | R^{a}X | R^{b}III | R⁷VI |
| R³III | R^{a}X | R^{b}III | R⁷VII |
| R³III | R^{a}X | R^{b}III | R⁷VIII |
| R³III | R^{a}X | R^{b}III | R⁷IX |
| R³III | R^{a}X | R^{b}IV | — |
| R³III | R^{a}X | R^{b}V | — |
| R³III | R^{a}X | R^{b}VI | — |
| R³I | — | R^{b}VII | — |
| R³II | — | R^{b}VII | — |
| R³III | — | R^{b}VII | — |

The compounds of the present invention can be produced, for example, by the following processes.

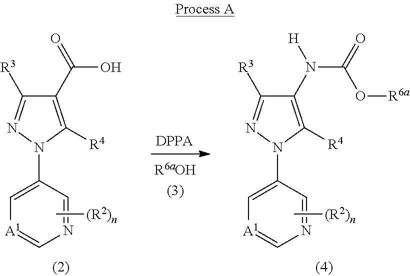

A compound represented by the formula (2) [wherein A¹, R², R³, R⁴ and n are the same as defined above] is reacted with diphenylphosphoryl azide (DPPA) and a compound represented by the formula (3) [wherein R^{6a} is the same as defined above] to obtain a compound of the present invention represented by the formula (4) [wherein A¹, R², R³, R⁴, R^{6a} and n are the same as defined above].

In the reaction, DPPA can be used in an amount of from 0.5 to 50 equivalents per 1 equivalent of the compound represented by the formula (2), and the compound represented by the formula (3) can be used in an amount of 1 equivalent to an amount sufficient as a solvent per 1 equivalent of the compound represented by the formula (2). If necessary, a base such as potassium carbonate, triethylamine, pyridine or 4-(dimethylamino)pyridine may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon such as pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (3) are known compounds, and some of them are commercially available. The rest of them can be readily synthesized from known compounds by known methods disclosed in the literature.

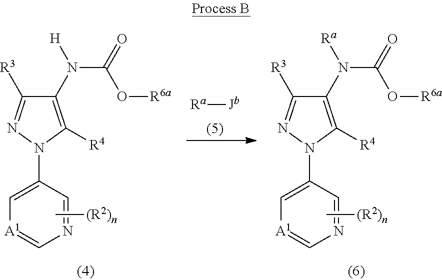

A compound represented by the formula (4) obtainable by Process A is reacted with a compound represented by the formula (5) [wherein R^{a} is the same as defined above, J^{b} is a leaving group such as a halogen atom, —OH, —OSO₂Me or —OSO₂CF₃] to obtain a compound of the present invention represented by the formula (6) [wherein A¹, R², R³, R⁴, R^{6a}, R^{a} and n are the same as defined above].

In the reaction, the compound represented by the formula (5) is used in an amount of from 0.5 to 50 equivalents per 1 equivalent of the compound represented by the formula (4). If necessary, an acid such as hydrochloric acid, sulfuric acid or p-toluenesulfonic acid or a base such as potassium carbonate, triethylamine, pyridine, 4-(dimethylamino)pyridine, sodium hydride, sodium hydroxide or potassium hydroxide or a Mitsunobu reaction using diethylazodicarboxylate, triphenylphosphine and the like may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (5) are known compounds, and some of them are commercially available. The rest of them can be readily synthesized from known compounds by known methods disclosed in the literature.

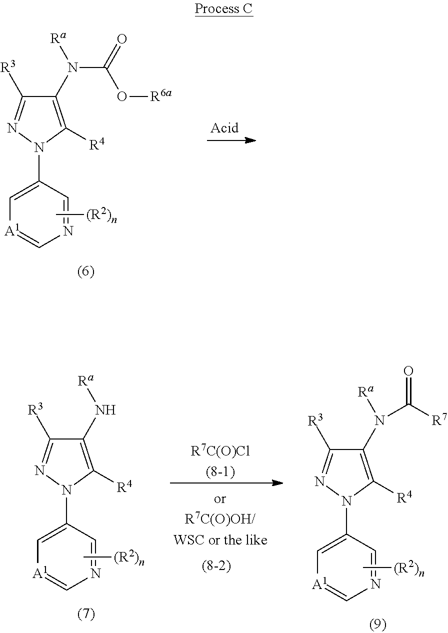

A compound represented by the formula (6) obtainable by Process B is reacted with an acid to obtain a compound represented by the formula (7) [wherein A¹, R², R³, R⁴, R^{a} and n are the same as defined above].

As the acid, hydrochloric acid, sulfuric acid, p-toluenesulfonic acid, trifluoroacetic acid or the like may be used.

The reaction may be carried out without a solvent or may be carried out in a solvent such as a polar solvent like acetonitrile or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

The compound represented by the formula (7) thus obtained is reacted with a compound represented by the formula (8-1) [wherein R⁷ is the same as defined above] to obtain a compound of the present invention represented by the formula (9) [wherein A¹, R², R³, R⁴, R⁷, R^{a} and n are the same as defined above]. In the reaction, the compound represented by the formula (8-1) is used in an amount of from 0.5 to 50 equivalents per 1 equivalent of the compound represented by the formula (7). If necessary, a base such as potassium carbonate, triethylamine, pyridine or 4-(dimethylamino)pyridine may be used. The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (8-1) are known compounds, and some of them are commercially available. The rest of them can be readily synthesized from known compounds by known methods disclosed in the literature.

A compound represented by the formula (7) may also be reacted with a compound represented by the formula (8-2) [wherein R⁷ is the same as defined above] in the presence of a condensation agent to obtain a compound of the present invention represented by the formula (9). In the reaction, a condensation agent such as WSC {1-ethyl-3-(3-dimethylaminopropyl)carbodiimide hydrochloride} or CDI (carbonyldiimidazole) is used in an amount of from 1 to 4 equivalents per 1 equivalent of the compound represented by the formula (8-2). The compound represented by the formula (8-2) is used in an amount of from 0.5 to 50 equivalents per 1 equivalent of the compound (7). If necessary, a base such as potassium carbonate, triethylamine, pyridine or 4-(dimethylamino)pyridine may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (8-2) are known compounds, and some of them are commercially available. The rest of them can be readily synthesized from known compounds by known methods disclosed in the literature.

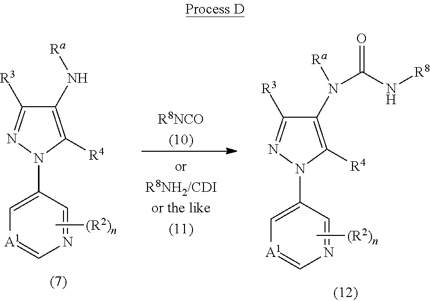

A compound represented by the formula (7) obtainable by Process C is reacted with a compound represented by the formula (10) [wherein R⁸ is the same as defined above] to obtain a compound of the present invention represented by the formula (12) [wherein A¹, R², R³, R⁴, R⁸, R^{a} and n are the same as defined above]. In the reaction, the compound represented by the formula (10) is used in an amount of from 0.5 to 50 equivalents per 1 equivalent of the compound represented by the formula (7). If necessary, a base such as potassium carbonate, triethylamine, pyridine or 4-(dimethylamino)pyridine may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (5) are known compounds, and some of them are commercially available. The rest of them can be readily synthesized from known compounds by known methods disclosed in the literature.

A compound represented by the formula (7) may also be reacted with a compound represented by the formula (11) [wherein R⁸ is the same as defined above] to obtain a compound represented by the formula (12).

In the reaction, CDI (carbonyldiimidazole), bis(trichloromethyl) carbonate or the like is used in an amount of from 1 to 4 equivalents per 1 equivalent of the compound represented by the formula (11). The compound represented by the formula (11) is used in an amount of from 0.5 to 50 equivalents per 1 equivalent of the compound represented by the formula (7). If necessary, a base such as potassium carbonate, triethylamine, pyridine or 4-(dimethylamino)pyridine may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (11) are known compounds, and some of them are commercially available. The rest of them can be readily synthesized from known compounds by known methods disclosed in the literature.

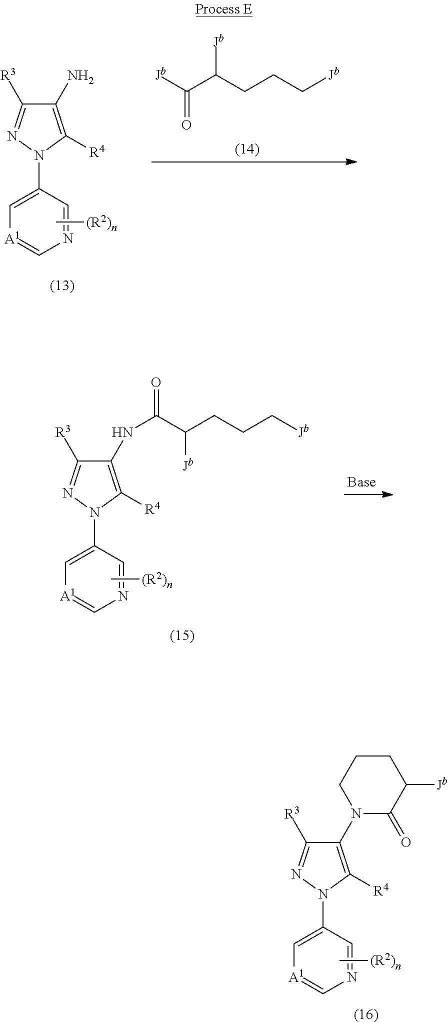

A compound represented by the formula (13) [wherein A¹, R², R³, R⁴ and n are the same as defined above] is reacted with a compound represented by the formula (14) [wherein J^{b} is the same as defined above] in the same manner as in Process C to obtain a compound of the present invention represented by the formula (15) [wherein A¹, R², R³, R⁴, J^{b} and n are the same as defined above].

The compounds represented by the formula (13) can be produced by a known method disclosed, for example, in Farmaco, Edizione Scientifica, (1970), 25(8), 592. Some of the compounds represented by the formula (14) [wherein J^{b} is the same as defined above] are commercially available. The rest of them can be readily synthesized from known compounds by known methods disclosed in the literature such as Tetrahetron: Asymmetry, 2003, vol. 14, pp. 2587.

The compound represented by the formula (15) is reacted with a base such as potassium carbonate, triethylamine, pyridine or 4-(dimethylamino)pyridine to obtain a compound represented by the formula (16) [wherein A¹, R², R³, R⁴, J^{b} and n are the same as defined above]. The base is used in an amount of from 0.5 to 50 equivalents per 1 equivalent of the compound represented by the formula (15).

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

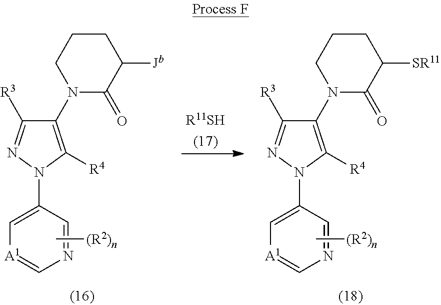

A compound represented by the formula (16) obtainable by Process E is reacted with a compound represented by the formula (17) [wherein R¹¹ is the same as defined above] to obtain a compound of the present invention represented by the formula (18) [wherein A¹, R², R³, R⁴, R¹¹ and n are the same as defined above]. In the reaction, the compound represented by the formula (17) is used in an amount of from 0.5 to 50 equivalent s per 1 equivalent of the compound represented by the formula (16). If necessary, an acid such as hydrochloric acid, sulfuric acid or p-toluenesulfonic acid or a base such as potassium carbonate, triethylamine, pyridine, 4-(dimethylamino)pyridine, sodium hydride, sodium hydroxide or potassium hydroxide may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (17) are known compounds, and some of them are commercially available. The rest of them can be readily synthesized from known compounds by known methods disclosed in the literature.

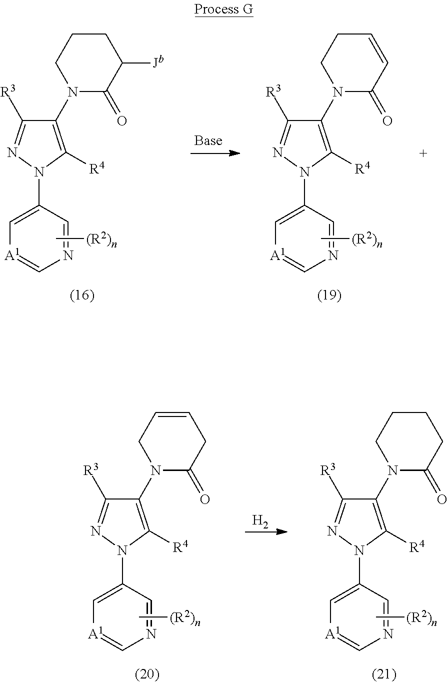

A compound represented by the formula (16) obtainable by Process E is reacted with a base to obtain a compound of the present invention represented by the formula (19) [wherein A¹, R², R³, R⁴ and n are the same as defined above] and a compound of the present invention represented by the formula (20) [wherein A¹, R², R³, R⁴ and n are the same as defined above].

As the base, potassium carbonate, triethylamine, pyridine, 4-(dimethylamino)pyridine, 1,8-diazabicyclo[5.4.0]-7-undecene, sodium hydroxide, potassium hydroxide or the like may be used.

The reaction may be carried out without a solvent or may be carried out in a solvent such as a polar solvent like acetonitrile or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Then, the compound represented by the formula (19) or the compound represented by the formula (20) is hydrogenated in the presence of a catalyst to obtain a compound of the present invention represented by the formula (21) [wherein A¹, R², R³, R⁴ and n are the same as defined above]. As the catalyst, a palladium-activated carbon catalyst, a platinum-activated carbon catalyst, Lindlar's catalyst or the like may be used.

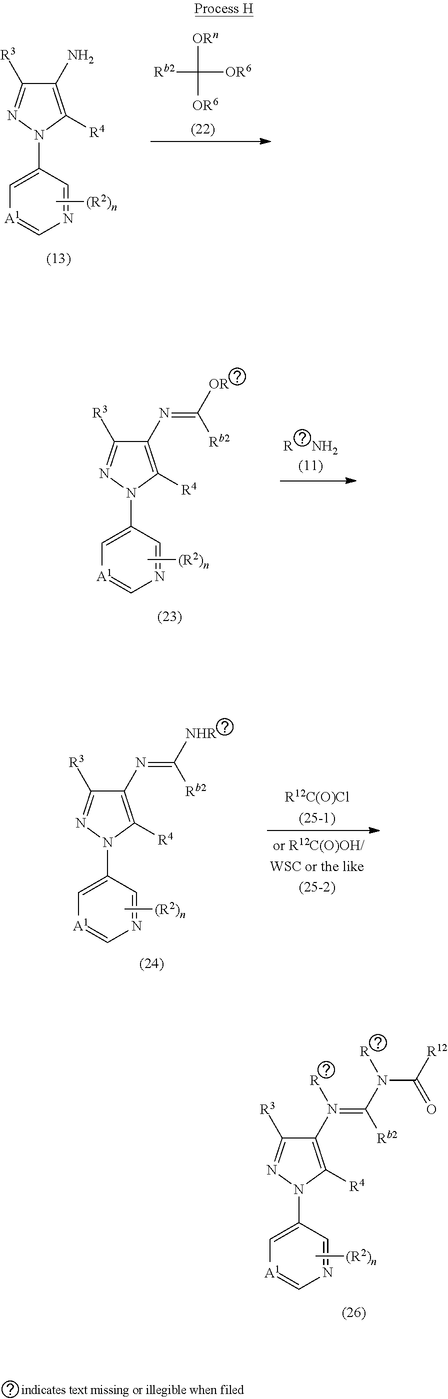

A compound represented by the formula (13) is reacted with a compound represented by the formula (22) [wherein R^{b2} and R⁶ are the same as defined above] to obtain a compound of the present invention represented by the formula (23) [wherein A¹, R², R³, R⁴, R^{b2}, R⁶ and n are the same as defined above]. The compound represented by the formula (22) is used in an amount of from 0.5 equivalent to an amount sufficient as a solvent, per 1 equivalent of the compound represented by the formula (13). If necessary, an acid such as hydrochloric acid, sulfuric acid or p-toluenesulfonic acid may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (22) are known compounds, and some of them are commercially available. The rest of them can be readily synthesized from known compounds by known methods disclosed in the literature.

The compound represented by the formula (23) is then reacted with a compound represented by the formula (11) to obtain a compound of the present invention represented by the formula (24) [wherein A¹, R², R³, R⁴, R^{8a}, R^{b2} and n are the same as defined above]. The compound represented by the formula (11) is used in an amount of from 0.5 equivalent to 50 equivalents per 1 equivalent of the compound represented by the formula (23). If necessary, an acid such as hydrochloric acid, sulfuric acid or p-toluenesulfonic acid or a base such as potassium carbonate, triethylamine, pyridine or 4-(dimethylamino)pyridine may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

The compound of the present invention represented by the formula (24) thus obtained is reacted with a compound represented by the formula (25-1) [wherein R¹² is the same as defined above] or a compound represented by the formula (25-2) [wherein R¹² is the same as defined above] in the same manner as in Process C to obtain a compound of the present invention represented by the formula (26) [wherein A¹, R², R³, R⁴, R^{8a}, R¹², R^{b2} and n are the same as defined above].

Some of the compounds represented by the formulae (25-1) and (25-2) are known compounds, and some of them are commercially available. The rest of them can be readily synthesized from known compounds by known methods disclosed in the literature.

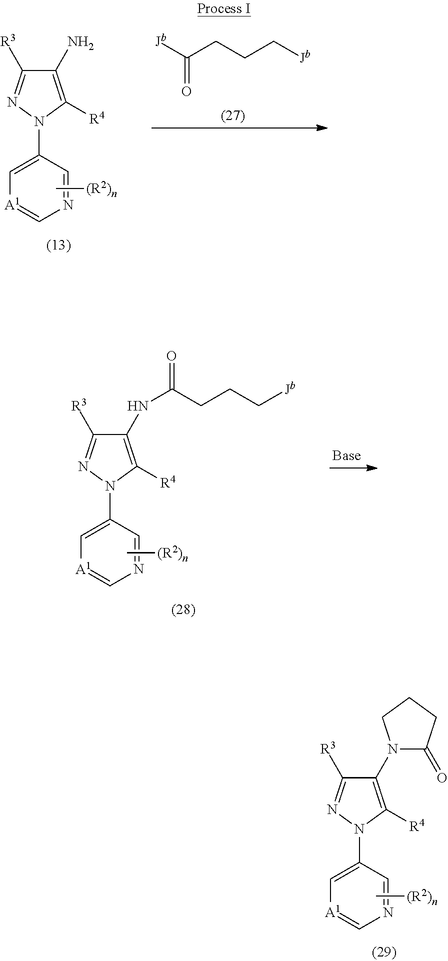

A compound represented by the formula (13) is reacted with a compound represented by the formula (27) [wherein J^{b} is the same as defined above] in the same manner as in Process to obtain a compound of the present invention represented by the formula (28) [wherein A¹, R², R³, R⁴, J^{b} and n are the same as defined above].

Some of the compounds represented by the formula (27) are known compounds, and some of them are commercially available. The rest of them can be readily synthesized from known compounds by known methods disclosed in the literature.

The compound represented by the formula (28) is reacted with a base such as sodium hydride, potassium carbonate, triethylamine, pyridine or 4-(dimethylamino)pyridine in the same manner as in Process E to obtain a compound of the present invention represented by the formula (29) [wherein A¹, R², R³, R⁴ and n are the same as defined above].

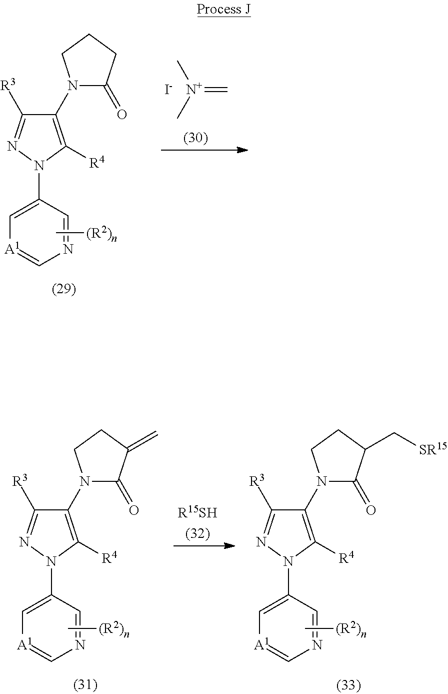

A compound represented by the formula (29) is reacted with dimethylmethyleneammonium iodide (Eschenmoser's reagent) to obtain a compound of the present invention represented by the formula (31) [wherein A¹, R², R³, R⁴ and n are the same as defined above].

Dimethylmethyleneammonium iodide is used in an amount of from 0.5 equivalent to 50 equivalents per 1 equivalent of the compound represented by the formula (29). If necessary, an acid such as trimethylsilyl trifluoromethanesulfonate or a base such as lithium bis(trimethylsilyl)amide may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

The compound of the present invention represented by the formula (31) thus obtained is reacted with a compound represented by the formula (32) [wherein R¹⁵ is the same as defined above] in the same manner as in Process F to obtain a compound of the present invention represented by the formula (33) [wherein A¹, R², R³, R⁴, R¹⁵ and n are the same as defined above].

Some of the compounds represented by the formula (17) are known compounds, and some of them are commercially available. The rest of them can be readily synthesized from known compounds by known methods disclosed in the literature.

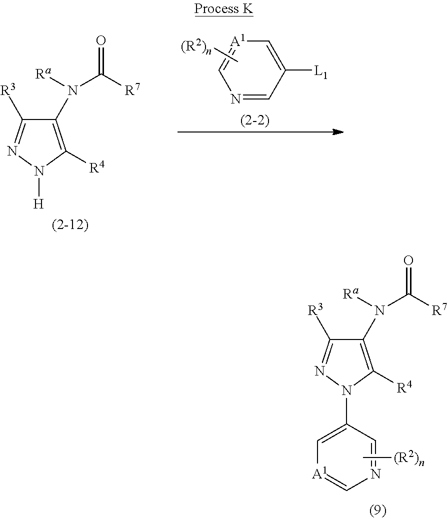

A compound represented by the formula (2-12) [wherein R³, R⁴, R⁷ and R^{a} are the same as defined above] is reacted with a compound represented by the formula (2-2) [wherein A¹, R² and n are the same as defined above, and L₁ is a fluorine atom, a chlorine atom, a bromine atom, an iodine atom, a —B(OH)₂ group or a —B(OR⁵¹)₂ group (wherein R⁵¹ is a hydrogen atom or identical or different C₁-C₆ alkyl, or two R⁵¹'s may form —CH₂CH₂— or —C(CH₃)₂C(CH₃)₂— together with each other)] in the presence of a catalyst and a base to obtain a compound of the present invention represented by the formula (9).

The compound represented by the formula (2-2) is used in an amount of from 0.8 to 5 equivalent s per 1 equivalent of the compound represented by the formula (2-12).

Some of the compounds represented by the formula (2-2) used herein are known compounds, and some of them are commercially available. The rest of them can be readily synthesized from known compounds by known methods disclosed in the literature.

In the reaction, as the catalyst, for example, a palladium catalyst such as palladium-carbon, palladium chloride, palladium acetate, bis(triphenylphosphine)palladium chloride or tetrakis(triphenylphosphine)palladium or a copper catalyst such as metal copper, copper (I) acetate, copper (II) acetate, copper (I) oxide, copper(II) oxide or copper iodide may be used. The catalyst may be used in an amount of from 0.001 to 1.0 equivalent, preferably from 0.01 to 0.5 equivalent, more preferably from 0.05 to 0.2 equivalent per 1 equivalent of the compound represented by the formula (2-12).

As the base, a tertiary amine compound such as pyridine, diisopropylethylamine or triethylamine, an inorganic base such as sodium hydroxide, potassium hydroxide, sodium carbonate, potassium carbonate, cesium carbonate or sodium hydrogen carbonate or the like may be used. The base is used in an amount of from 0.1 to 10.0 equivalents per 1 equivalent of the compound represented by the formula (2-12).

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

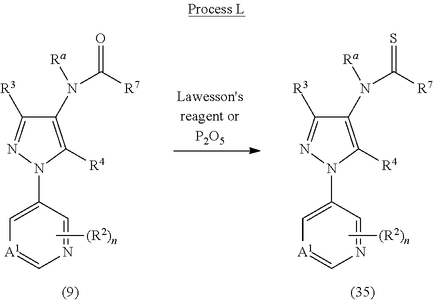

A compound represented by the formula (9) and a sulfidizing agent such as phosphorus pentasulfide, phosphorus pentasulfide-HMDO (hexamethyldisiloxane) or Lawesson's Reagent (2,4-bis(4-methoxyphenyl)-1,3,2,4-dithiadiphosphetane-2,4-disulfide) are reacted to obtain a compound of the present invention represented by the formula (35) [wherein A¹, R², R³, R⁴, R⁷, R^{a} and n are the same as defined above].

In the reaction, the sulfidizing agent is used in an amount of from 0.5 to 50 equivalents per 1 equivalent of the compound represented by the formula (9).

If necessary, a base such as potassium carbonate, triethylamine, pyridine or 4-(dimethylamino)pyridine may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

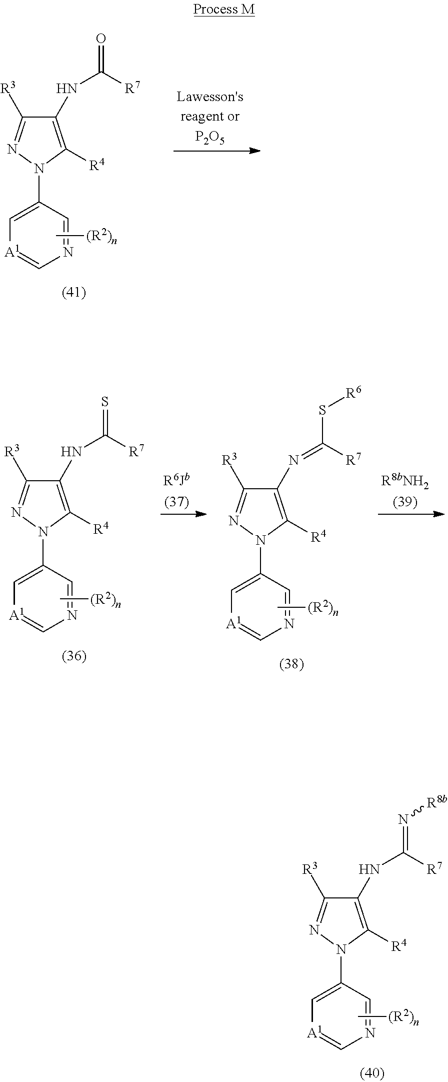

From a compound represented by the formula (41) [wherein A¹, R², R³, R⁴, R⁷ and n are the same as defined above], a compound of the present invention represented by the formula (36) [wherein A¹, R², R³, R⁴, R⁷ and n are the same as defined above] can be obtained in the same manner as in Process L.

Then, the compound represented by the formula (36) is reacted with a compound represented by the formula (37) [wherein R⁶ and J^{b} are the same as defined above] to obtain a compound of the present invention represented by the formula (38) [wherein A¹, R², R³, R⁴, R⁶, R⁷ and n are the same as defined above].

In the reaction, the compound represented by the formula (37) is used in an amount of from 0.5 to 50 equivalents per 1 equivalent of the compound represented by the formula (36). If necessary, an acid such as hydrochloric acid, sulfuric acid or p-toluenesulfonic acid or a base such as potassium carbonate, triethylamine, pyridine, 4-(dimethylamino)pyridine, sodium hydride, sodium hydroxide or potassium hydroxide or a Mitsunobu reaction using diethylazodicarboxylate, triphenylphosphine and the like may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (37) are known compounds, and some of them are commercially available. The rest of them can be readily synthesized from known compounds by known methods disclosed in the literature.

Then, the compound represented by the formula (38) is reacted with a compound represented by the formula (39) [wherein R^{8b} is the same as defined above] to obtain a compound of the present invention represented by the formula (40) [wherein A¹, R², R³, R⁴, R⁷, R^{8b} and n are the same as defined above].

In the reaction, the compounds represented by the formula (39) is used in an amount of from 0.5 to 50 equivalents per 1 equivalent of the compounds represented by the formula (38). If necessary, an acid such as hydrochloric acid, sulfuric acid or p-toluenesulfonic acid or a base such as potassium carbonate, triethylamine, pyridine, 4-(dimethylamino)pyridine, sodium hydride, sodium hydroxide or potassium hydroxide may be used.

The reaction may be carried out without a solvent or may be carried out in a solvent such as a polar solvent like N,N-dimethylformamide, N,N-dimethylacetamide, acetonitrile, dimethyl sulfoxide, 1,3-dimethyl-2-imidazolinone or water, an alcohol like methanol, ethanol, propanol, 2-propanol or ethylene glycol, an ether like diethyl ether, tetrahydrofuran or diphenyl ether, an aromatic hydrocarbon like benzene, toluene or xylene, a halohydrocarbon like methylene chloride, chloroform or carbon tetrachloride, or an aliphatic hydrocarbon like pentane or n-hexane. These solvents may be used alone or in combinations of two or more.

The reaction temperature may be set arbitrarily within the range of from −60° C. to the refluxing temperature of the reaction mixture, and the reaction time may be set arbitrarily within the range of from 5 minutes to 100 hours, though it depends on the concentrations of the reactants and the reaction temperature.

Some of the compounds represented by the formula (39) are known compounds, and some of them are commercially available. The rest of them can be readily synthesized from known compounds by known methods disclosed in the literature.

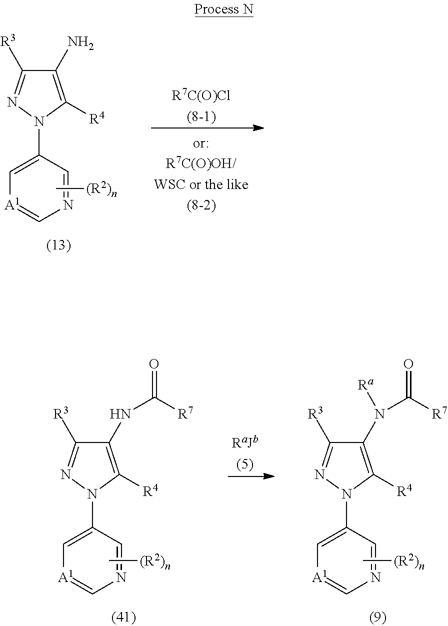

A compound represented by the formula (13) [wherein A¹, R², R³, R⁴ and n are the same as defined above] is reacted with a compound represented by the formula (8-1) or a compound represented by the formula (8-2) in the same manner as in Process C to obtain a compound of the present invention represented by the formula (41) [wherein A¹, R², R³, R⁴, R⁷ and n are the same as defined above].

Then, the compound represented by the formula (41) is reacted with a compound represented by the formula (5) in the same manner as in Process B to obtain a compound of the present invention represented by the formula (9).

If necessary, the reactions in Processes A to N may be carried out in an atmosphere of an inert gas such as nitrogen or argon.

In Processes A to N, the reaction mixture after a reaction can be worked up by an ordinary procedure such as direct concentration, concentration of a solution in an organic solvent after washing with water, pouring into ice-water or extraction with an organic solvent followed by concentration to obtain the desired product. If purification is needed, the desired product may be isolated or purified by recrystallization or fractionation by column chromatography, thin layer chromatography or liquid chromatography.

The compound represented by the formula (2) used in Process A may be synthesized, for example, as follows.

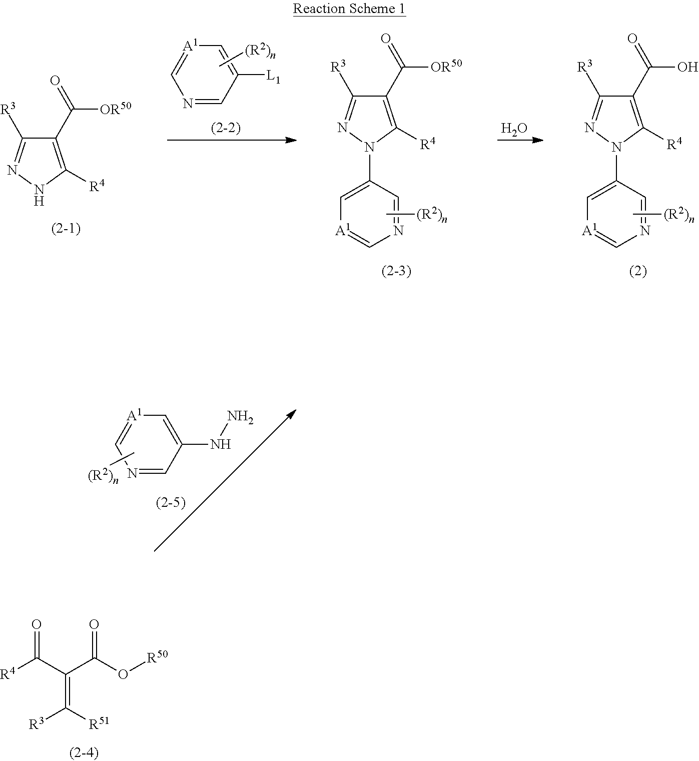

A compound represented by the formula (2-1) [wherein R³ and R⁴ are the same as defined above, R⁵⁰ is a C₁-C₆ alkyl group] can be synthesized by a known method disclosed in Synlett, 2004, vol. 4, p. 703 or the like.

Namely, the compound represented by the formula (2-1) is reacted with a known compound represented by the formula (2-2) [wherein A¹, R² and n are the same as defined above, L₁ is a fluorine atom, a chlorine atom, a bromine atom, an iodine atom, a —B(OH)₂ group or a —B(OR⁵¹)₂ group (wherein R⁵¹ is a hydrogen atom or identical or different C₁-C₆ alkyl, or two R⁵¹'s may form —CH₂CH₂— or —C(CH₃)₂C(CH₃)₂— together with each other] in the presence of a metal catalyst such as copper and a base or in the presence of a transition metal catalyst such palladium and a base by a method disclosed in the literature such as Journal of Organic Chemistry, 2004, vol. 69, p. 5578 to obtain a compound represented by the formula (2-3) [wherein A¹, R², R³, R⁴ and n are the same as defined above, R⁵⁰ is a C₁-C₆ alkyl group].

A compound represented by the formula (2-3) is also obtained by reacting a compound represented by the formula (2-4) [wherein R³, R⁴ and R⁵⁰ are the same as defined above, R⁵¹ is a leaving group such as a methoxy group, an ethoxy group or a dimethylamino group] and a compound represented by the formula (2-5) [wherein A¹, R² and n are the same as defined above] a method disclosed in the literature such as Journal of Heterocyclic Chemistry, 1987, vol. 24, p. 1669. The compound represented by the formula (2-4) can be synthesized by a known method disclosed in Journal of Heterocyclic Chemistry, 1987, vol. 24, p. 693, and the compound represented by the formula (2-5) by a known method disclosed in Journal of Medicinal Chemistry, 2002, vol. 45, p. 5397.

The compound represented by the formula (2-3) may be hydrolyzed by a known method disclosed in the literature to obtain a compound represented by the formula (2) [wherein A¹, R², R³, R⁴ and n are the same as defined above].

The compound represented by the formula (2-12) used in Process K may be synthesized, for example, as follows.

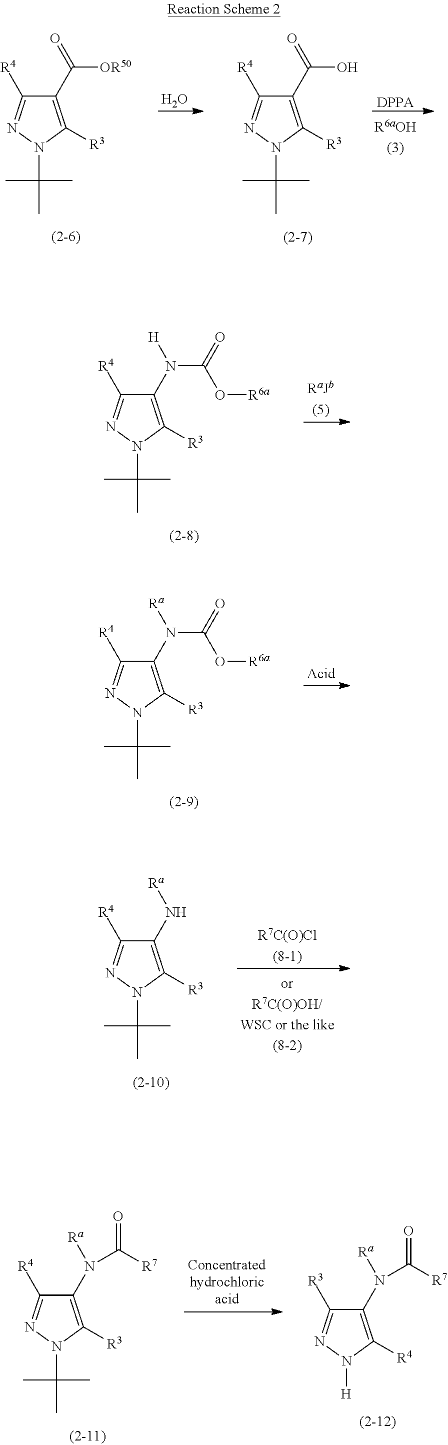

A compound represented by the formula (2-6) [wherein R³, R⁴ and R⁵⁰ are the same as defined above] can be synthesized by a known method disclosed in Journal of Heterocyclic Chemistry, 1987, vol. 24, p. 693.

The compound represented by the formula (2-6) may be hydrolyzed by a known method in the literature to obtain a compound represented by the formula (2-7) [wherein R³ and R⁴ are the same as defined above].

Then, from the compound represented by the formula (2-7), a compound represented by the formula (2-8) [wherein R³, R⁴ and R^{6a} are the same as defined above] in the same manner as in Process A.

Then, the compound represented by the formula (2-8) is reacted with a compound represented by the formula (5) in the same manner as in Process B to obtain a compound represented by the formula (2-9) [wherein R³, R⁴, R^{a} and R^{6a} are the same as defined above].

Further, from the compound represented by the formula (2-9), a compound represented by the formula (2-11) [wherein R³, R⁴, R⁷ and R^{a} are the same as defined above] can be synthesized by Process C through a compound represented by the formula (2-10) [wherein R³, R⁴ and R^{a} are the same as defined above].

The compound represented by the formula (2-11) thus obtained is deprotected in concentrated hydrochloric acid to obtain a compound represented by the formula (2-12) [wherein R³, R⁴, R⁷ and R^{a} are the same as defined above].

The compound represented by the formula (7) used in Process D may be synthesized, for example, as follows.

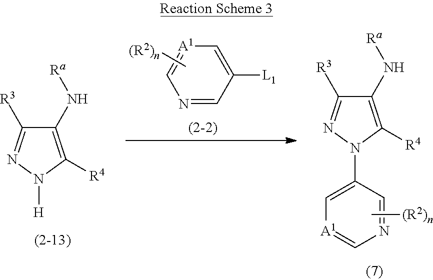

A compound represented by the formula (2-13) [wherein R³, R⁴ and R^{a} are the same as defined above] can be synthesized by a known method disclosed in WO2011/048082 or the like.

A compound represented by the formula (2-13) and a compound represented by the formula (2-2) are reacted in the same manner as in Process K to obtain a compound represented by the formula (7).

As specific active compounds covered by the present invention, for example, those shown in Tables 2 and 3 may be mentioned. However, the compounds merely exemplify the present invention, and the present invention is by no means restricted thereto. In the Tables, Me denotes methyl group, and similarly, Et denotes ethyl group, n-Pr and Pr-n denote normal propyl group, i-Pr and Pr-i denote isopropyl group, c-Pr and Pr-c denote cyclopropyl group, n-Bu and Bu-n denote normal butyl group, s-Bu and Bu-s denote secondary butyl group, i-Bu and Bu-i denote isobutyl group, t-Bu and Bu-t denote tertiary-butyl group, c-Bu and Bu-c denote cyclobutyl group, n-Pen and Pen-n denote normal pentyl group, c-Pen and Pen-c denote cyclopentyl group, n-Hex and Hex-n denote normal hexyl group, c-Hex and Hex-c denote cyclohexyl group, Hept denotes heptyl group, Oct denotes octyl group, and Ph denotes phenyl group.

In the Tables, D1-1a, D1-1b, D1-2a, D1-2b, D1-2c, D1-4-a, D1-4-b, D1-5a, D1-5b, D1-5c, D1-5d, D1-5e, D1-5f, D1-6a, D1-6b, D1-6c, D1-6d, D1-7a, D1-7b, D1-8a, D1-8b, D1-8c, D1-8f, D1-9a, D1-9b, D1-9c, D1-9e, D1-9f, D1-10a, D1-10b, D1-10c, D1-10e, D1-10f, D1-11a, D1-12a, D1-12b, D1-12c, D1-12f, D1-13a, D1-13b, D1-13d, D1-14a, D1-14b, D1-15a, D1-15b, D1-16a, D1-16b, D1-17a, D1-18a, D1-19a, D1-20a, D1-21a, D1-21b, D1-22a, D1-23a, D1-23b, D1-24a, D1-25a, D1-26a, D1-27a, D1-27b, D1-28a, D1-29a, D1-30a, D1-31a, D1-32a, D1-32b, D1-33a, D1-33b, D1-34a, D1-35a, D1-35b, D1-36a, D1-37a, D1-38a, D1-38b, D1-39a, D1-39b, D1-40a, D1-40b, D1-40c, D1-41a, D1-42a, D1-43a, D1-45a, D1-45d, D1-49a, D1-51a, D1-59a, D1-61a, D1-79a, D1-80a, D1-81a, D1-81b, D1-82a, D1-82b, D1-82c, D1-84d, D1-85d, D1-87a, D1-88a, D1-88b, D1-92c, D1-93a, D1-94b, D1-94c, D1-98a, D1-103b, D1-103c, D1-103d, D1-103e, D1-103f, D1-103g, D1-103h, D1-103i, D1-108a and, D1-108b represent the following structures, and the numbers in the structural formulae D1-1b, D1-2c, D1-5d, D1-5e, D1-5f, D1-6d, D1-7b, D1-8f, D1-9e, D1-9f, D1-10e, D1-10f, D1-12f, D1-32b, D1-33b, D1-38b, D1-39b indicate the positions of the substituent X¹, the numbers in the structural form a D1-45d indicate the positions of the substituent X^{1b}, and the numbers in the structural formula D1-108b indicate the positions of the substituent Z.

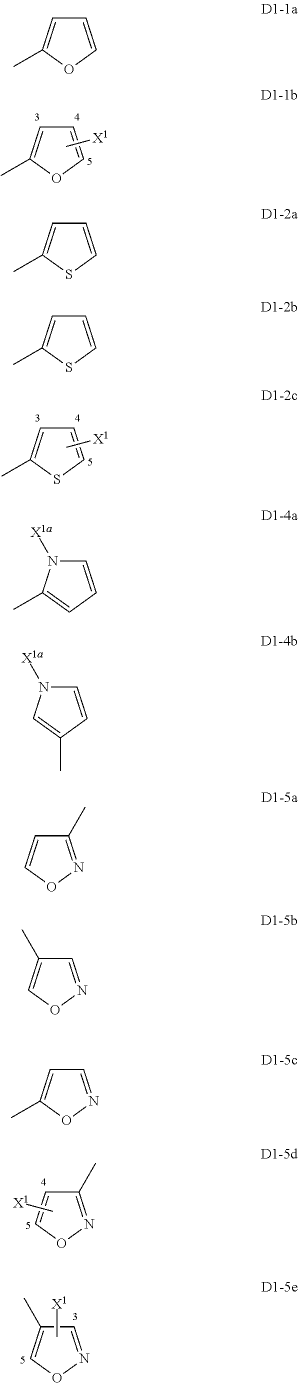

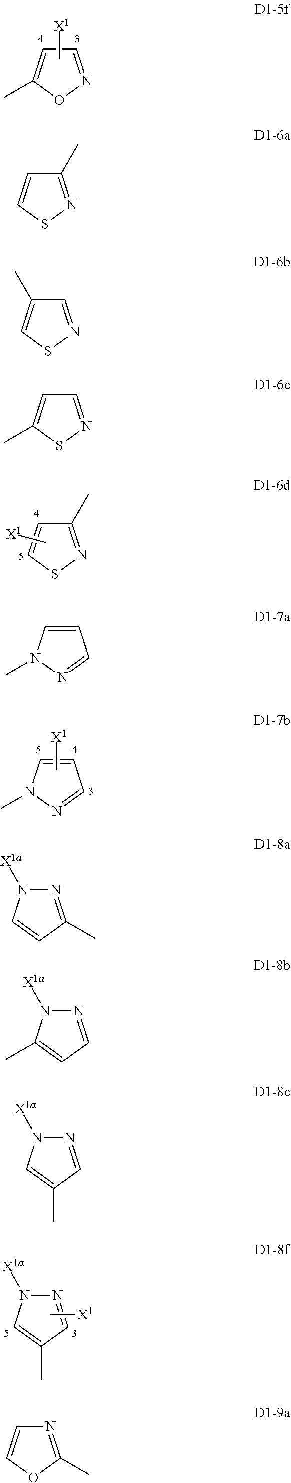

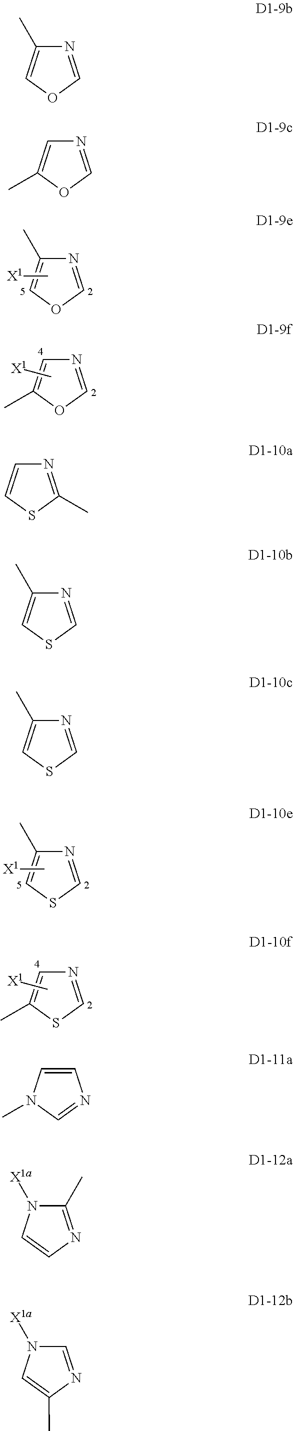

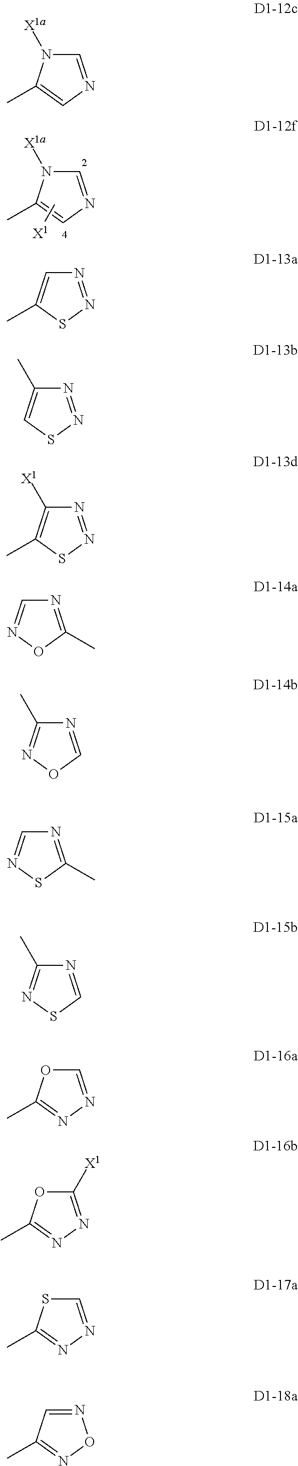

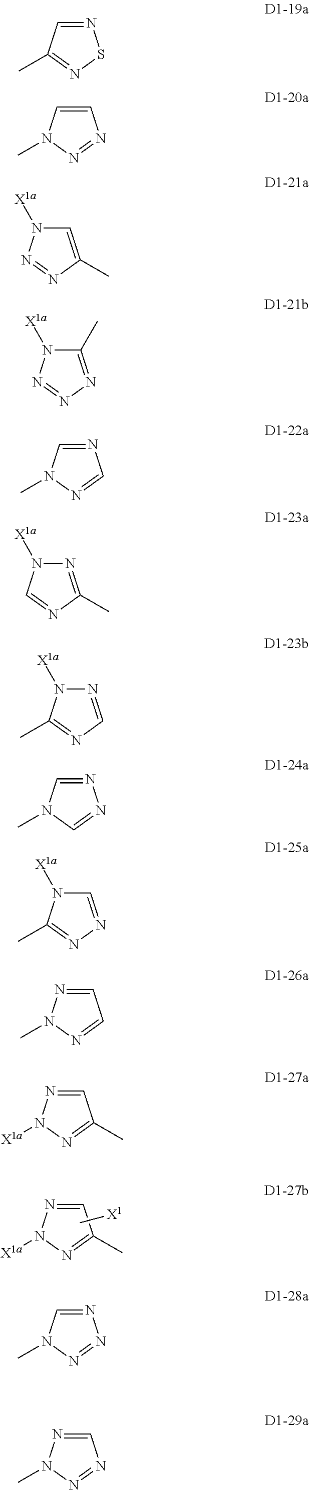

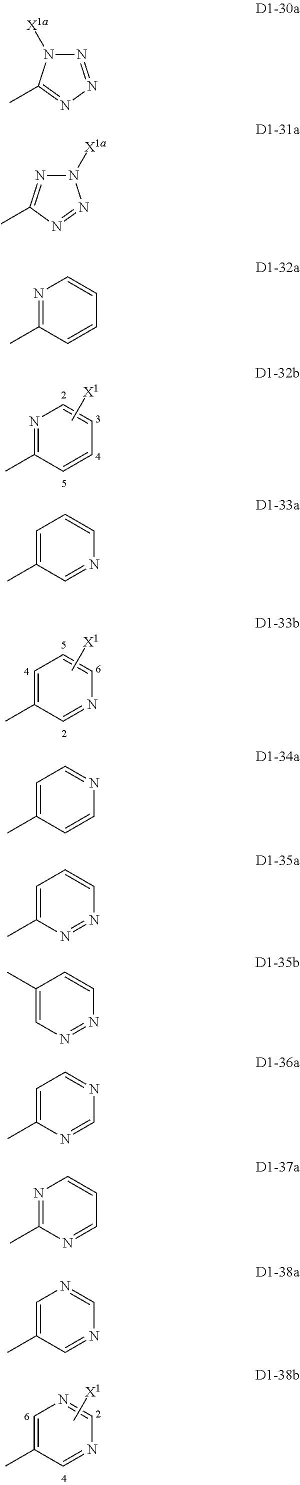

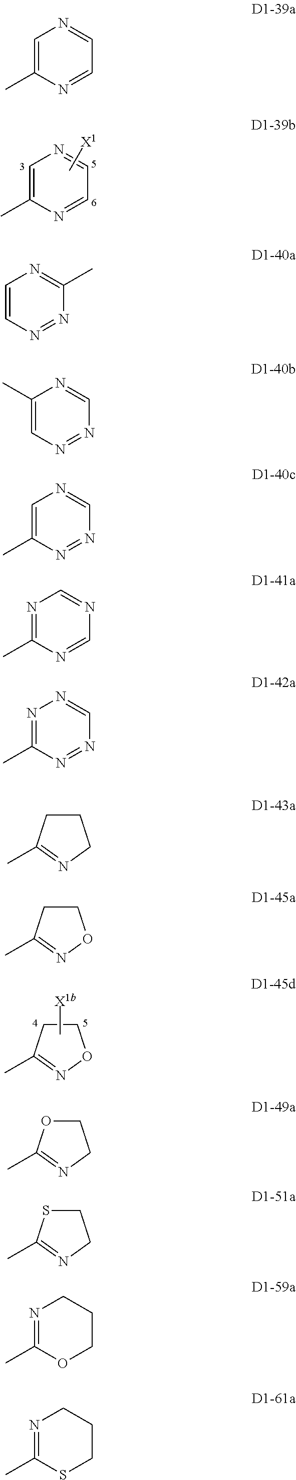

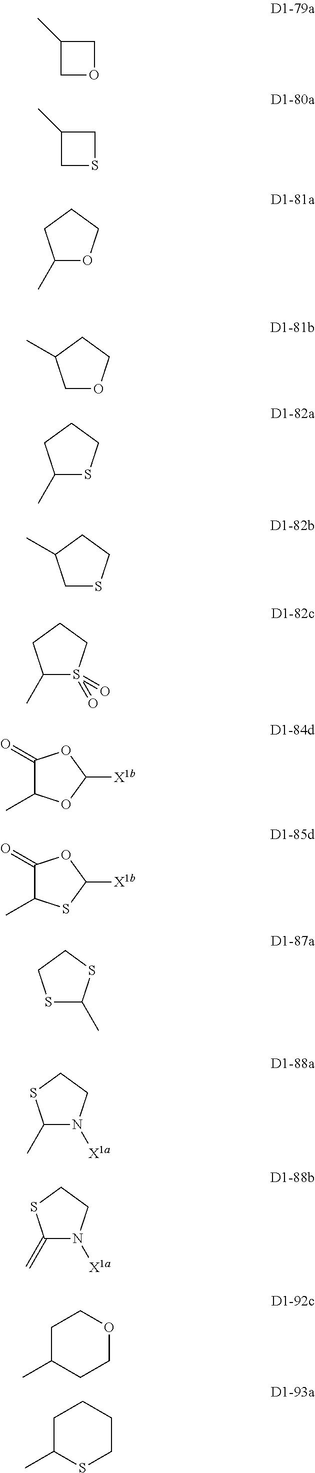

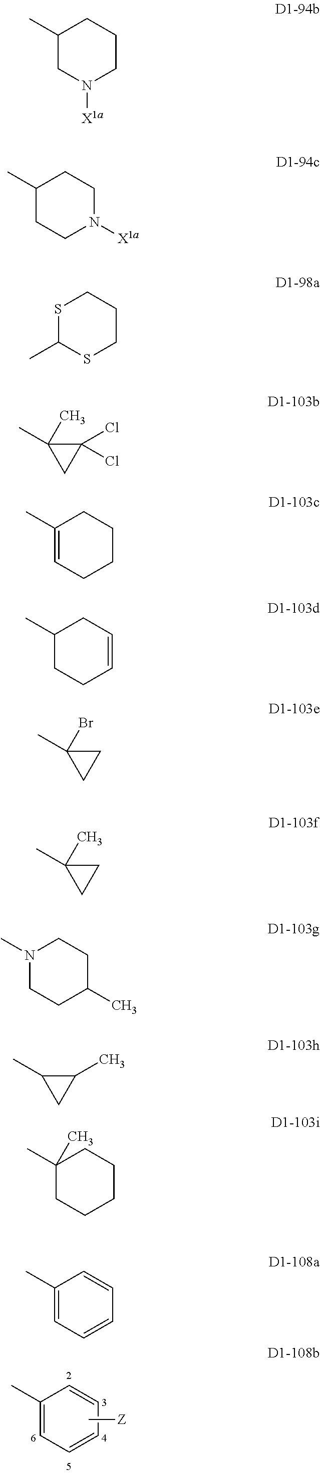

TABLE 2

| 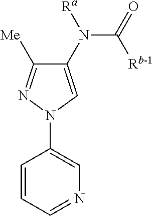 | |
|---|---|
| 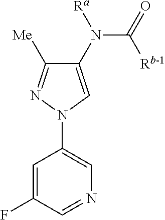 | |
| 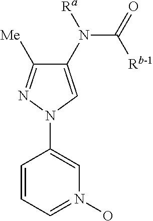 | |
| 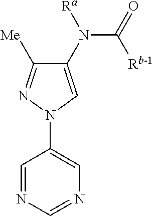 | |
| 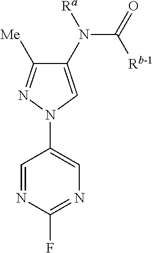 | |
| 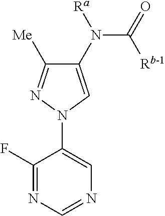 | |
| 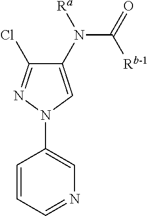 | |
| 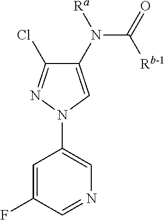 | |
| 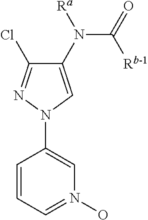 | |
| 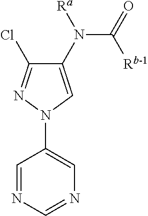 | |
| 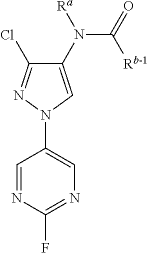 | |
| 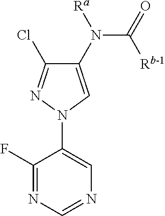 | |
| 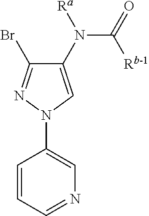 | |
| 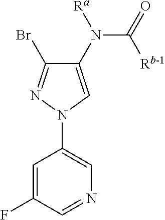 | |
| 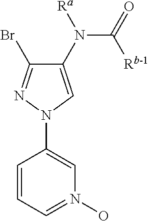 | |
| 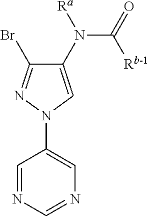 | |
| 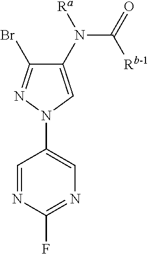 | |
| 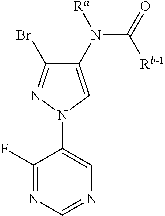 | |
| 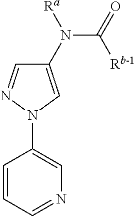 | |
| 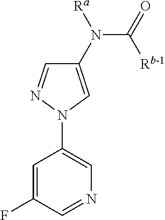 | |
| 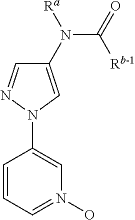 | |
| 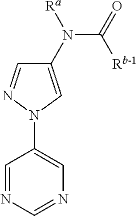 | |
| 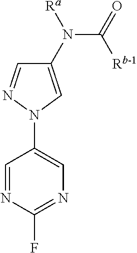 | |
| 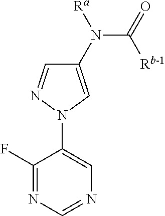 | |
| 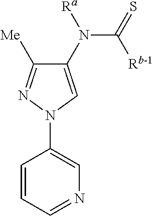 | |
| 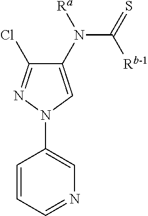 | |
| 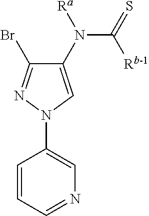 | |
| 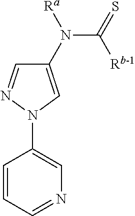 | |
| 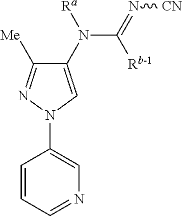 | |
| 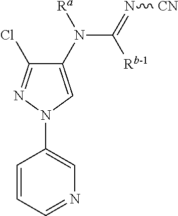 | |
| 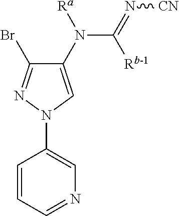 | |
| 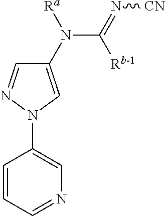 | |
| 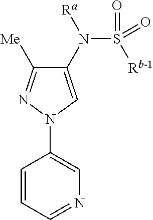 | |
| 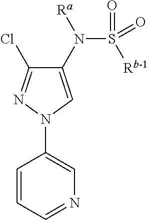 | |
| 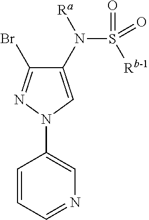 | |
| 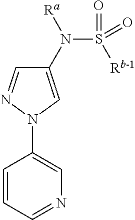 | |
| 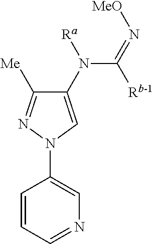 | |
| 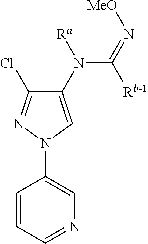 | |
| 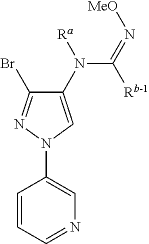 | |
| 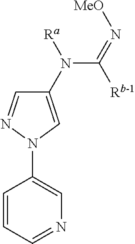 | |
| 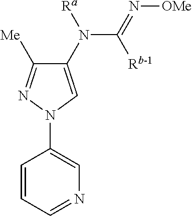 | |
| 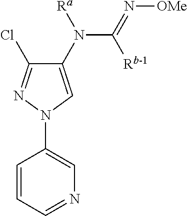 | |
| 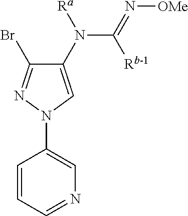 | |
| 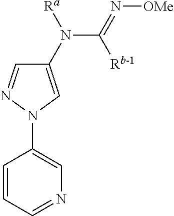 | |
| 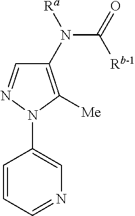 | |
| 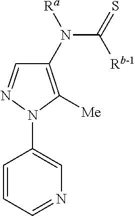 | |
| 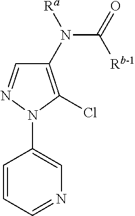 | |
| 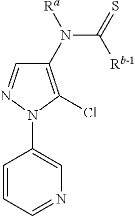 | |
| 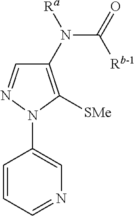 | |
| 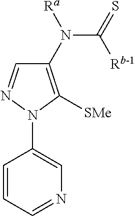 | |
| 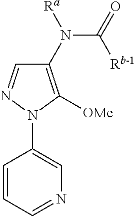 | |
| 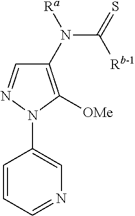 | |
| 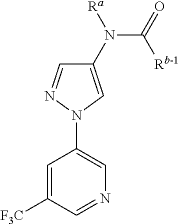 | |
| 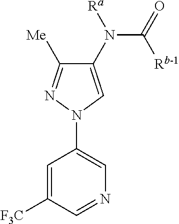 | |
| 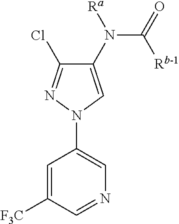 | |
| 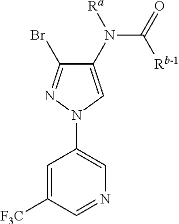 | |
| 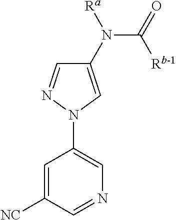 | |
| 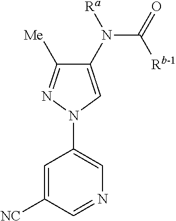 | |
| 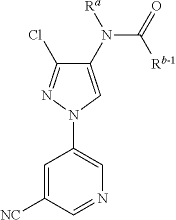 | |
| 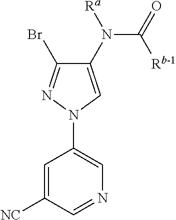 | |
| 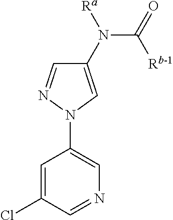 | |
| 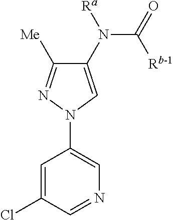 | |
| 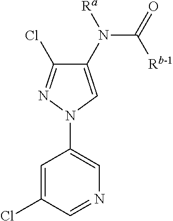 | |
| 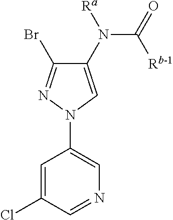 | |
| 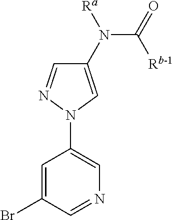 | |
| 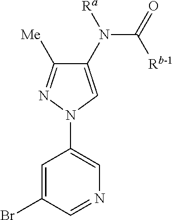 | |
| 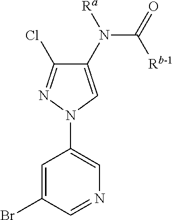 | |
| 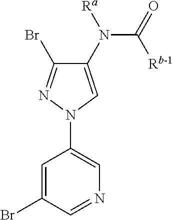 | |
| 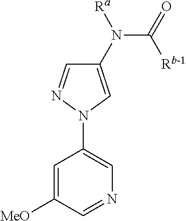 | |
| 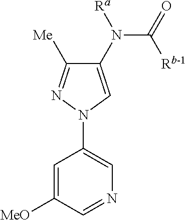 | |
| 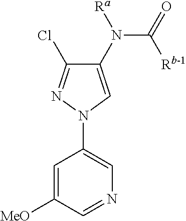 | |
| 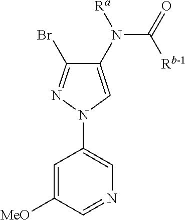 | |
| 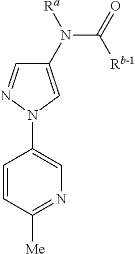 | |
| 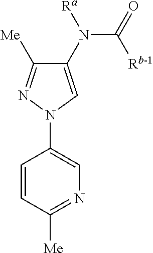 | |
| 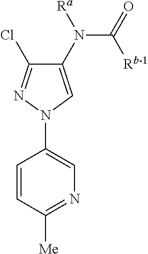 | |
| 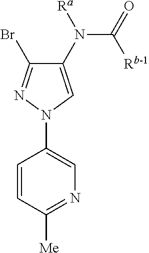 | |
| 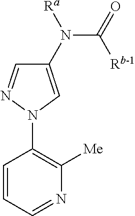 | |
| 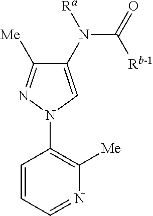 | |
| 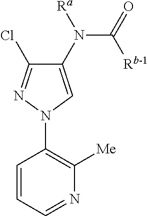 | |
| 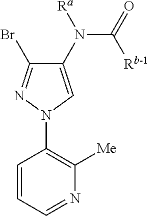 | |
| 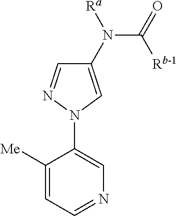 | |
| 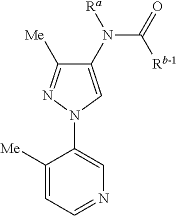 | |
| 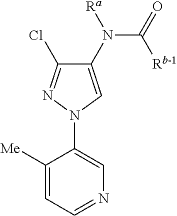 | |
| 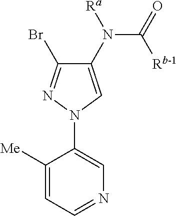 | |
| 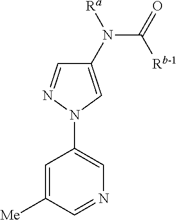 | |
| 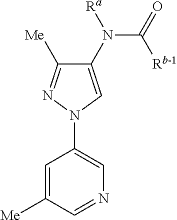 | |
| 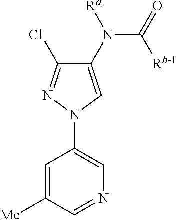 | |
| 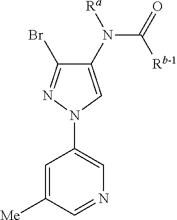 | |
| 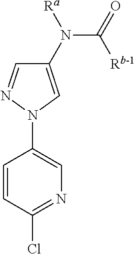 | |
| 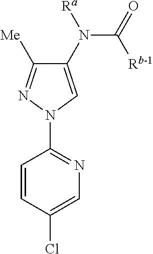 | |
| 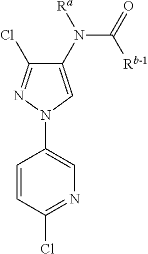 | |
| 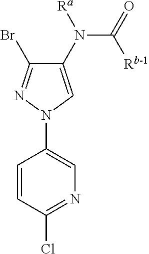 | |
| 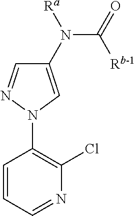 | |
| 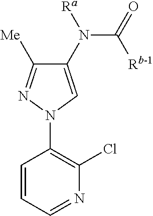 | |
| 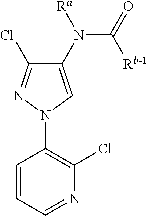 | |
| 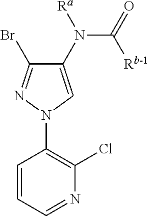 | |
| 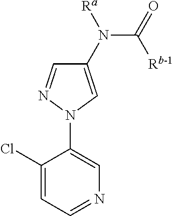 | |
| 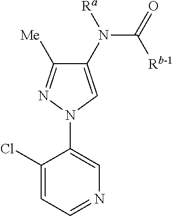 | |
| 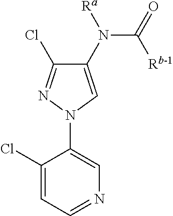 | |
| 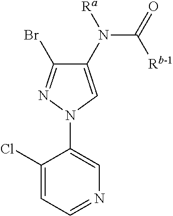 | |
| 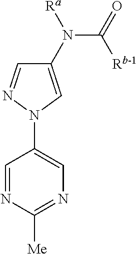 | |
| 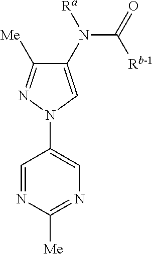 | |
| 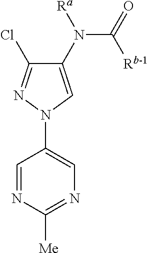 | |
| 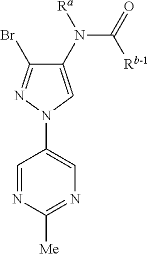 | |
| 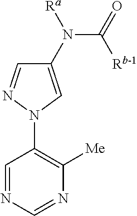 | |
| 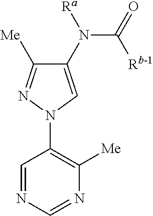 | |
| 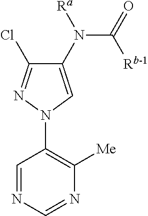 | |
| 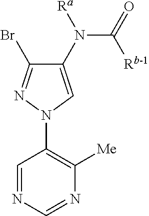 | |
| 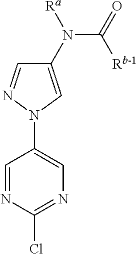 | |
| 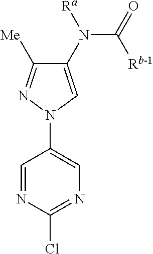 | |
| 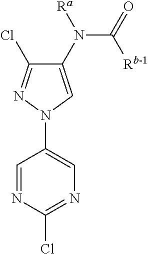 | |
| 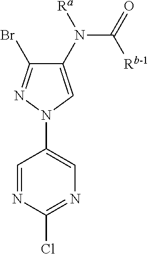 | |
| 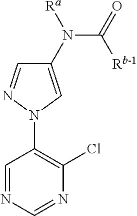 | |
| 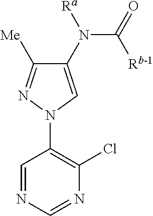 | |
| 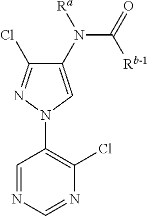 | |
| 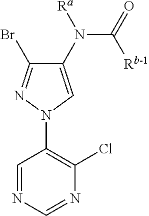 | |
| 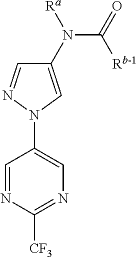 | |
| 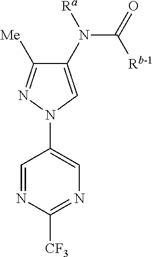 | |
| 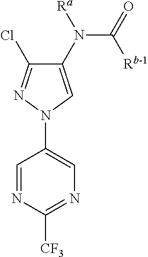 | |
| 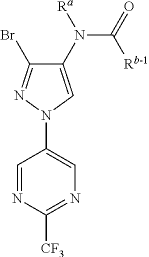 | |
| 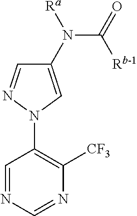 | |
| 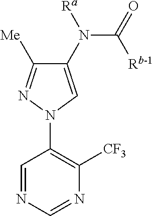 | |
| 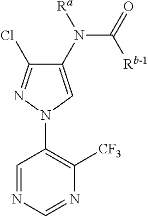 | |
| 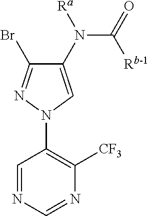 | |
| 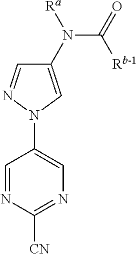 | |
| 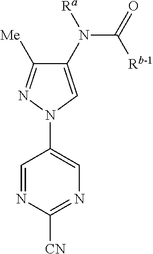 | |
| 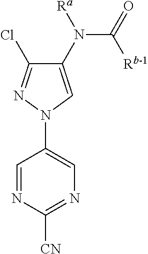 | |
| 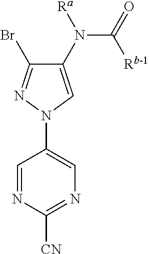 | |
| 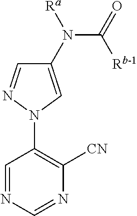 | |
| 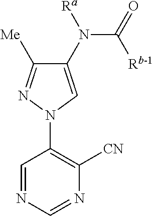 | |
| 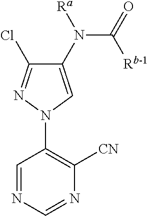 | |
| 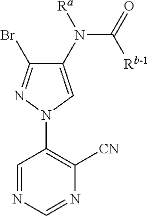 | |
| 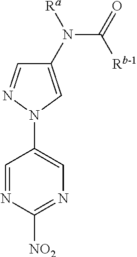 | |
| 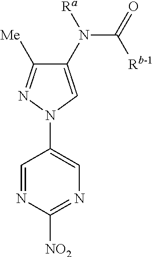 | |
| 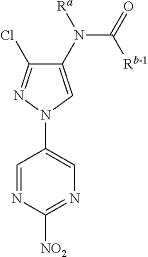 | |
| 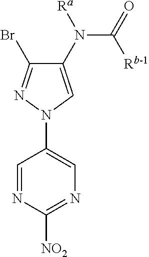 | |
| 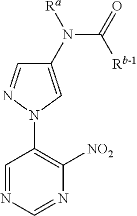 | |
| 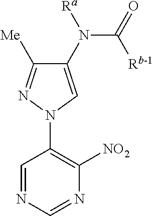 | |
| 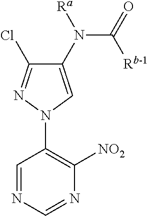 | |
| 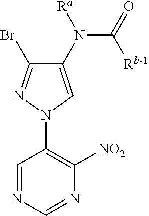 | |
| 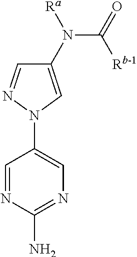 | |
| 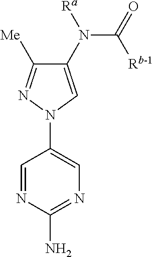 | |
| 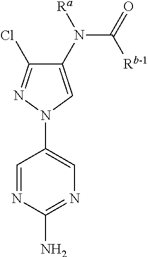 | |
| 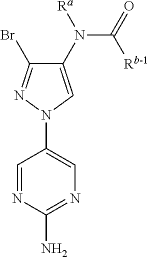 | |
| 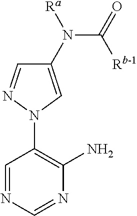 | |
| 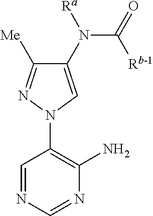 | |
| 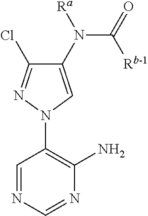 | |
| 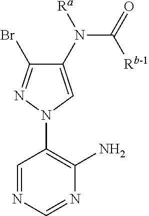 | |
| 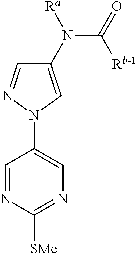 | |
| 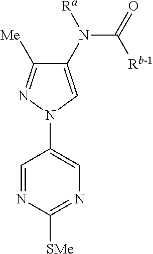 | |
| 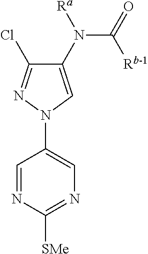 | |
| 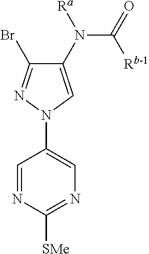 | |
| 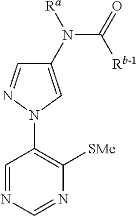 | |
| 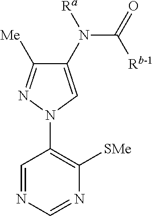 | |
| 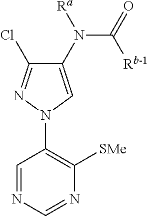 | |
| 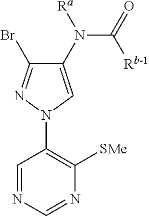 | |
| 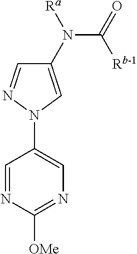 | |
| 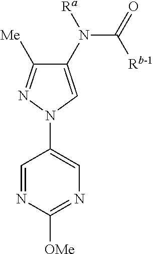 | |
| 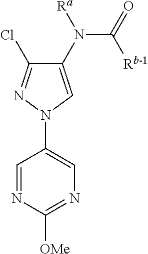 | |
| 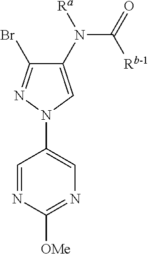 | |
| 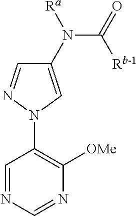 | |
| 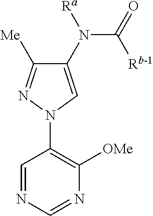 | |
| 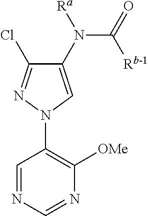 | |
| 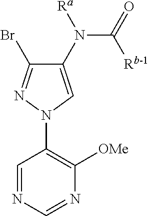 | |
| R^{a} | R^{b-1} |
| H | C(O)Me |
| H | Me |
| H | Et |
| H | Pr-n |
| H | Pr-i |
| H | Pr-c |
| H | Bu-n |
| H | Bu-c |
| H | Bu-i |
| H | Bu-s |
| H | Bu-t |
| H | Pen-n |
| H | Pen-c |
| H | Hex-n |
| H | Hex-c |
| H | CH₂Br |
| H | CH(Br)Me |
| H | CH₂CH₂CH₂Cl |
| H | CH(Br)CH₂CH₂CH₂Br |
| H | CH₂CF₃ |
| H | CH₂CH(OH)CF₃ |
| H | CH₂CN |
| H | CH₂C(O)OMe |
| H | CH₂C(O)NHMe |
| H | CH═CH₂ |
| H | CH═CHSMe |
| H | CH═CHCF₃ |
| H | CH═C(Me)CF₃ |
| H | CH{D1-108b(4-CF₃)} |
| H | C≡CH |
| H | C≡CSiMe₃ |
| H | CH₂OMe |
| H | CH₂OEt |
| H | CH(Me)OCH₂CF₃ |
| H | CH(Me)ON═CHMe |
| H | CH₂CH₂SC(O)Me |
| H | CH₂OC(O)Me |
| H | CH₂SMe |
| H | CH₂S(O)Me |
| H | CH₂S(O)₂Me |
| H | CH₂SEt |
| H | CH₂S(O)Et |
| H | CH₂S(O)₂Et |
| H | CH₂SCH₂CH═CH₂ |
| H | CH₂S(O)CH₂CH═CH₂ |
| H | CH₂S(O)₂CH₂CH═CH₂ |
| H | CH₂SCH₂CF₃ |
| H | CH₂S(O)CH₂CF₃ |
| H | CH₂S(O)₂CH₂CF₃ |
| H | CH(Me)SMe |
| H | CH(Me)S(O)Me |
| H | CH(Me)S(O)₂Me |
| H | CH(Me)SEt |
| H | CH(Me)S(O)Et |
| H | CH(Me)S(O)₂Et |
| H | CH(Me)S(═NCN)Et |
| H | CH(Me)S(O)(═NCN)Et |
| H | CH(Me)SPr-n |
| H | CH(Me)S(O)Pr-n |
| H | CH(Me)S(O)₂Pr-n |
| H | CH(Me)SPr-i |
| H | CH(Me)S(O)Pr-i |
| H | CH(Me)S(O)₂Pr-i |
| H | CH(Me)SBu-t |
| H | CH(Me)S(O)Bu-t |
| H | CH(Me)S(O)₂Bu-t |
| H | CH(Me)SCH₂Pr-c |
| H | CH(Me)S(O)CH₂Pr-c |
| H | CH(Me)S(O)₂CH₂Pr-c |
| H | CH(Me)SCH₂OMe |
| H | CH(Me)SCH₂SMe |
| H | CH(Me)S(O)CH₂SMe |
| H | CH(Me)S(O)₂CH₂SMe |
| H | CH(Me)SCH₂C≡CH |
| H | CH(Me)S(O)CH₂C≡CH |
| H | CH(Me)S(O)₂CH₂C≡CH |
| H | CH(Me)SCH₂C(O)NHMe |
| H | CH(Me)S(O)CH₂C(O)NHMe |
| H | CH(Me)S(O)₂CH₂C(O)NHMe |
| H | CH(Me)SCH₂C(O)OMe |
| H | CH(Me)S(O)CH₂C(O)OMe |
| H | CH(Me)S(O)₂CH₂C(O)OMe |
| H | CH(Me)SCH₂CH₂CH₂Cl |
| H | CH(Me)S(O)CH₂CH₂CH₂Cl |
| H | CH(Me)S(O)₂CH₂CH₂CH₂Cl |
| H | CH(Me)SCH₂CF₃ |
| H | CH(Me)S(O)CH₂CF₃ |
| H | CH(Me)S(O)₂CH₂CF₃ |
| H | CH(Me)SCH₂(D1-34a) |
| H | CH(Me)S(O)CH₂(D1-34a) |
| H | CH(Me)S(O)₂CH₂(D1-34a) |
| H | CH(CH₃)SCH₂Si(CH₃)₃ |
| H | CH(Me)SCN |
| H | CH(Me)SCH₂CN |
| H | CH(Me)SC(O)Me |
| H | CH(Me)S(D1-37a) |
| H | CH(Et)SMe |
| H | CH(Et)S(O)Me |
| H | CH(Et)S(O)₂Me |
| H | CH(Et)SEt |
| H | CH(Et)S(O)Et |
| H | CH(Et)S(O)₂Et |
| H | CH(F)SMe |
| H | CH(F)S(O)Me |
| H | CH(F)S(O)₂Me |
| H | CH(F)SEt |
| H | CH(F)S(O)Et |
| H | CH(F)S(O)₂Et |
| H | CH{OC(O)Me}SMe |
| H | CH{OC(O)Me}S(O)Me |
| H | CH{OC(O)Me}S(O)₂Me |
| H | CH(SMe)₂ |
| H | C(Me)₂SMe |
| H | C(Me)₂S(O)Me |
| H | C(Me)₂S(O)₂Me |
| H | CH₂CH₂SH |
| H | CH₂CH₂SMe |
| H | CH₂CH₂S(O)Me |
| H | CH₂CH₂S(O)₂Me |
| H | CH₂CH₂SEt |
| H | CH₂CH₂S(O)Et |
| H | CH₂CH₂S(O)₂Et |
| H | CH₂CH₂SPr-n |
| H | CH₂CH₂S(O)Pr-n |
| H | CH₂CH₂S(O)₂Pr-n |
| H | CH₂CH₂SPr-i |
| H | CH₂CH₂S(O)Pr-i |
| H | CH₂CH₂S(O)₂Pr-i |
| H | CH₂CH₂SCH₂CF₃ |
| H | CH₂CH₂S(O)CH₂CF₃ |
| H | CH₂CH₂S(O)₂CH₂CF₃ |
| H | CH₂CH₂SC(O)Me |
| H | CH(Me)CH₂SMe |
| H | CH(Me)CH₂S(O)Me |
| H | CH(Me)CH₂S(O)₂Me |
| H | CH(Me)CH₂SEt |
| H | CH(Me)CH₂S(O)Et |
| H | CH(Me)CH₂S(O)₂Et |
| H | CH(Me)CH₂SPr-n |
| H | CH(Me)CH₂S(O)Pr-n |
| H | CH(Me)CH₂S(O)₂Pr-n |
| H | CH(Me)CH₂SPr-i |
| H | CH(Me)CH₂S(O)Pr-i |
| H | CH(Me)CH₂S(O)₂Pr-i |
| H | CH(Me)CH₂SCH₂CF₃ |
| H | CH(Me)CH₂S(O)CH₂CF₃ |
| H | CH(Me)CH₂S(O)₂CH₂CF₃ |
| H | CH(Me)CH₂SC(O)Me |
| H | CH(Et)CH₂SMe |
| H | CH(Et)CH₂S(O)Me |
| H | CH(Et)CH₂S(O)₂Me |
| H | CH(OMe)CH₂SMe |
| H | CH(OMe)CH₂S(O)Me |
| H | CH(OMe)CH₂S(O)₂Me |
| H | CH(SMe)CH₂SMe |
| H | CH(SMe)CH₂S(O)Me |
| H | CH(SMe)CH₂S(O)₂Me |
| H | CH(CH₂SMe)CH₂SMe |
| H | CH(CH₂SMe)CH₂S(O)Me |
| H | CH(CH₂SMe)CH₂S(O)₂Me |
| H | CH(Cl)CH₂SMe |
| H | CH(Cl)CH₂S(O)Me |
| H | CH(Cl)CH₂S(O)₂Me |
| H | CH(CN)CH₂SMe |
| H | CH(CN)CH₂S(O)Me |
| H | CH(CN)CH₂S(O)₂Me |
| H | CH(CN)CH₂SEt |
| H | CH(CN)CH₂S(O)Et |
| H | CH(CN)CH₂S(O)₂Et |
| H | CH{C(O)OMe}CH₂SMe |
| H | CH{C(O)OMe}CH₂S(O)Me |
| H | CH{C(O)OMe}CH₂S(O)₂Me |
| H | CH{C(O)OMe}CH₂SEt |
| H | CH{C(O)OMe}CH₂S(O)Et |
| H | CH{C(O)OMe}CH₂S(O)₂Et |
| H | CH(Ph)CH₂SMe |
| H | CH(Ph)CH₂S(O)Me |
| H | CH(Ph)CH₂S(O)₂Me |
| H | C(Me)₂CH₂SMe |
| H | C(Me)₂CH₂S(O)Me |
| H | C(Me)₂CH₂S(O)₂Me |
| H | C(═CH₂)CH₂SMe |
| H | C(═CH₂)CH₂S(O)Me |
| H | C(═CH₂)CH₂S(O)₂Me |
| H | C(═CH₂)CH₂SEt |
| H | C(═CH₂)CH₂S(O)Et |
| H | C(═CH₂)CH₂S(O)₂Et |
| H | CH₂CH₂CH₂SEt |
| H | CH₂CH₂CH₂S(O)Et |
| H | CH₂CH₂CH₂S(O)₂Et |
| H | CH(Me)CH₂CH₂SEt |
| H | CH(Me)CH₂CH₂S(O)Et |
| H | CH(Me)CH₂CH₂S(O)₂Et |
| H | CH₂CH(Me)CH₂SEt |
| H | CH₂CH(Me)CH₂S(O)Et |
| H | CH₂CH(Me)CH₂S(O)₂Et |
| H | CH₂CH₂CH(Me)SEt |
| H | CH₂CH₂CH(Me)S(O)Et |
| H | CH₂CH₂CH(Me)S(O)₂Et |
| H | CH₂CH₂CH₂SPr-n |
| H | CH₂CH₂CH₂S(O)Pr-n |
| H | CH₂CH₂CH₂S(O)₂Pr-n |
| H | CH(Me)CH₂CH₂SPr-n |
| H | CH(Me)CH₂CH₂S(O)Pr-n |
| H | CH(Me)CH₂CH₂S(O)₂Pr-n |
| H | CH₂CH(Me)CH₂SPr-n |
| H | CH₂CH(Me)CH₂S(O)Pr-n |
| H | CH₂CH(Me)CH₂S(O)₂Pr-n |
| H | CH₂CH₂CH₂CH₂SMe |
| H | CH₂CH₂CH₂CH₂S(O)Me |
| H | CH₂CH₂CH₂CH₂S(O)₂Me |
| H | CH₂CH₂CH₂CH₂SEt |
| H | CH₂CH₂CH₂CH₂S(O)Et |
| H | CH₂CH₂CH₂CH₂S(O)₂Et |
| H | CH₂CH₂CH₂CH₂SPr-n |
| H | CH₂CH₂CH₂CH₂S(O)Pr-n |
| H | CH₂CH₂CH₂CH₂S(O)₂Pr-n |
| H | CH₂CH(Me)SMe |
| H | CH₂OCH₂SMe |
| H | CH₂CH(SMe)₂ |
| H | C(═CH₂)CH₂C(O)OH |
| H | C(═NOMe)CH₃ |
| H | C(═NOMe)Et |
| H | C(═NOMe)Pr-n |
| H | C(═NOEt)CH₃ |
| H | C(═NOPr-n)CH₃ |
| H | C(═NOPr-i)CH₃ |
| H | C(═NOBu-n)CH₃ |
| H | C(═NOCH₂Ph)CH₃ |
| H | C(═NOMe)CH₂OCH₂CF₃ |
| H | C(═NOMe)CH₂SMe |
| H | C(═NOMe)CH₂S(O)Me |
| H | C(═NOMe)CH₂S(O)₂Me |
| H | C(═NOMe)CH₂SEt |
| H | C(═NOMe)CH₂S(O)Et |
| H | C(═NOMe)CH₂S(O)₂Et |
| H | C(═NOMe)CH₂SCF₃ |
| H | C(═NOMe)CH₂S(O)CF₃ |
| H | C(═NOMe)CH₂S(O)₂CF₃ |
| H | C(═NOMe)CH₂SCH₂CF₃ |
| H | C(═NOMe)CH₂S(O)CH₂CF₃ |
| H | C(═NOMe)CH₂S(O)₂CH₂CF₃ |
| H | C(═NOEt)CH₂SMe |
| H | C(═NOEt)CH₂S(O)Me |
| H | C(═NOEt)CH₂S(O)₂Me |
| H | C(═NOPr-n)CH₂SMe |
| H | C(═NOPr-n)CH₂S(O)Me |
| H | C(═NOPr-n)CH₂S(O)₂Me |
| H | C(═NOPr-i)CH₂SMe |
| H | C(═NOPr-i)CH₂S(O)Me |
| H | C(═NOPr-i)CH₂S(O)₂Me |
| H | C(═NOBu-n)CH₂SMe |
| H | C(═NOBu-n)CH₂S(O)Me |
| H | C(═NOBu-n)CH₂S(O)₂Me |
| H | C(═NOCH₂Ph)CH₂SMe |
| H | C(═NOCH₂Ph)CH₂S(O)Me |
| H | C(═NOCH₂Ph)CH₂S(O)₂Me |
| H | C(═NOCH₂CH═CH₂)CH₂SMe |
| H | C(═NOCH₂OMe)CH₂SMe |
| H | C(═NOCH₂CH₂OMe)CH₂SMe |
| H | C{═NOCH₂CH₂Si(Me)₃}CH₂SMe |
| H | CH₂CH(═NOMe) |
| H | CH₂C(═NOMe)Me |
| H | CH₂C(═NOMe)Et |
| H | CH₂C(═NOMe)Pr-n |
| H | CH₂C(═NOMe)Pr-i |
| H | CH₂C(═NOMe)Bu-n |
| H | CH₂C(═NOMe)Bu-i |
| H | CH₂C(═NOMe)Bu-s |
| H | CH₂C(═NOMe)Bu-t |
| H | CH₂C(═NOMe)CF₃ |
| H | CH₂C(═NOCH₃)CH₂SCH₃ |
| H | CH(SMe)C(═NOMe)Me |
| H | CH{S(O)Me}C(═NOMe)Me |
| H | CH{S(O)₂Me}C(═NOMe)Me |
| H | CH(SEt)C(═NOMe)Me |
| H | CH{S(O)Et}C(═NOMe)Me |
| H | CH{S(O)₂Et}C(═NOMe)Me |
| H | CH₂NH₂ |
| H | CH₂NHC(O)Me |
| H | CH₂NHC(O)OMe |
| H | CH₂NHC(O)OBu-t |
| H | CH₂NHSO₂Me |
| H | CH₂N(Me)C(O)Me |
| H | CH₂N(Me)C(O)OMe |
| H | CH₂N(Me)C(O)OBu-t |
| H | CH₂N(Me)SO₂Me |
| H | CH(CH₂SMe)NH₂ |
| H | CH(CH₂SMe)NHC(O)Me |
| H | CH(CH₂SMe)NHC(O)OMe |
| H | CH(CH₂SMe)NHC(O)OBu-t |
| H | CH(CH₂SMe)NHSO₂Me |
| H | CH₂P(O)(OMe)₂ |
| H | CH₂P(O)(OEt)₂ |
| H | OBu-t |
| H | O{D1-108b(4-NO₂)} |
| H | SMe |
| H | NH₂ |
| H | NHCH₂CH₂SMe |
| H | N(Me)CH₂CH₂SMe |
| H | NHCH₂CH₂CH₂SMe |
| H | NHMe |
| H | NHEt |
| H | NPr-n |
| H | NHCH(Me)₂ |
| H | NHCH₂C═CH₂ |
| H | NHCH₂C(O)OMe |
| H | NHCH₂CF₃ |
| H | N(Me)CH₂CF₃ |
| H | NHC(O)OEt |
| H | NHC(O)CCl₃ |
| H | NHC(O)CH₂SMe |
| H | N(CH₂CH₃)C(O)CH₂SCH₃ |
| H | NHS(O)₂(D1-108a) |
| H | NHCH═NOMe |
| H | N═CHNMe₂ |
| H | NHPh |
| H | NH(D1-1a) |
| H | NH(D1-2a) |
| H | NH(D1-2b) |
| H | NH{D1-4a(X^{1a}═Me)} |
| H | NH{D1-4b(X^{1a}═Me)} |
| H | NH(D1-5a) |
| H | NH(D1-5b) |
| H | NH(D1-5c) |
| H | NH(D1-6a) |
| H | NH(D1-6b) |
| H | NH(D1-6c) |
| H | NH(D1-7a) |
| H | NH{D1-8a(X^{1a}═Me)} |
| H | NH{D1-8b(X^{1a}═Me)} |
| H | NH{D1-8c(X^{1a}═Me)} |
| H | NH(D1-9a) |
| H | NH(D1-9b) |
| H | NH(D1-9c) |
| H | NH(D1-10a) |
| H | NH(D1-10b) |
| H | NH(D1-10c) |
| H | NH(D1-11a) |
| H | NH{D1-12a(X^{1a}═Me)} |
| H | NH{D1-12b(X^{1a}═Me)} |
| H | NH{D1-12c(X^{1a}═Me)} |
| H | NH(D1-13a) |
| H | NH(D1-13b) |
| H | NH(D1-14a) |
| H | NH(D1-14b) |
| H | NH(D1-15a) |
| H | NH(D1-15b) |
| H | NH(D1-16a) |
| H | NH(D1-17a) |
| H | NH(D1-18a) |
| H | NH(D1-19a) |
| H | NH(D1-20a) |
| H | NH{D1-21a(X^{1a}═Me)} |
| H | NH{D1-21b(X^{1a}═Me)} |
| H | NH(D1-22a) |
| H | NH{D1-23a(X^{1a}═Me)} |
| H | NH{D1-23b(X^{1a}═Me)} |
| H | NH(D1-24a) |
| H | NH{D1-25a(X^{1a}═Me)} |
| H | NH(D1-26a) |
| H | NH{D1-27a(X^{1a}═Me)} |
| H | NH(D1-28a) |
| H | NH(D1-29a) |
| H | NH{D1-30a(X^{1a}═Me)} |
| H | NH{D1-31a(X^{1a}═Me)} |
| H | NH(D1-32a) |
| H | NH(D1-33a) |
| H | NH(D1-34a) |
| H | NH(D1-35a) |
| H | NH(D1-35b) |
| H | NH(D1-36a) |
| H | NH(D1-37a) |
| H | NH(D1-38a) |
| H | NH(D1-39a) |
| H | NH(D1-40a) |
| H | NH(D1-40b) |
| H | NH(D1-40c) |
| H | NH(D1-41a) |
| H | NH(D1-42a) |
| H | NH(D1-43a) |
| H | NH(D1-45a) |
| H | NH(D1-50a) |
| H | NH(D1-51a) |
| H | NH(D1-59a) |
| H | NH(D1-61a) |
| H | NH(D1-79a) |
| H | NH(D1-80a) |
| H | NH{D1-108b(2-Cl)} |
| H | NH{D1-108b(3-Cl)} |
| H | NH{D1-108b(4-Cl)} |
| H | NH{D1-108b(2-Me)} |
| H | NH{D1-108b(3-Me)} |
| H | NH{D1-108b(4-Me)} |
| H | N(Me)Ph |
| H | N(Me)(D1-1a) |
| H | N(Me)(D1-2a) |
| H | N(Me)(D1-2b) |
| H | N(Me){D1-4a(X^{1a}═Me)} |
| H | N(Me){D1-4b(X^{1a}═Me)} |
| H | N(Me)(D1-5a) |
| H | N(Me)(D1-5b) |
| H | N(Me)(D1-5c) |
| H | N(Me)(D1-6a) |
| H | N(Me)(D1-6b) |
| H | N(Me)(D1-6c) |
| H | N(Me)(D1-7a) |
| H | N(Me){D1-8a(X^{1a}═Me)} |
| H | N(Me){D1-8b(X^{1a}═Me)} |
| H | N(Me){D1-8c(X^{1a}═Me)} |
| H | N(Me)(D1-9a) |
| H | N(Me)(D1-9b) |
| H | N(Me)(D1-9c) |
| H | N(Me)(D1-10a) |
| H | N(Me)(D1-10b) |
| H | N(Me)(D1-10c) |
| H | N(Me)(D1-11a) |
| H | N(Me){D1-12a(X^{1a}═Me)} |
| H | N(Me){D1-12b(X^{1a}═Me)} |
| H | N(Me){D1-12c(X^{1a}═Me)} |
| H | N(Me)(D1-13a) |
| H | N(Me)(D1-13b) |
| H | N(Me)(D1-14a) |
| H | N(Me)(D1-14b) |
| H | N(Me)(D1-15a) |
| H | N(Me)(D1-15b) |
| H | N(Me)(D1-16a) |
| H | N(Me)(D1-17a) |
| H | N(Me)(D1-18a) |
| H | N(Me)(D1-19a) |
| H | N(Me)(D1-20a) |
| H | N(Me){D1-21a(X^{1a}═Me)} |
| H | N(Me){D1-21b(X^{1a}═Me)} |
| H | N(Me)(D1-22a) |
| H | N(Me){D1-23a(X^{1a}═Me)} |
| H | N(Me){D1-23b(X^{1a}═Me)} |
| H | N(Me)(D1-24a) |
| H | N(Me){D1-25a(X^{1a}═Me)} |
| H | N(Me)(D1-26a) |
| H | N(Me){D1-27a(X^{1a}═Me)} |
| H | N(Me)(D1-28a) |
| H | N(Me)(D1-29a) |
| H | N(Me){D1-30a(X^{1a}═Me)} |
| H | N(Me){D1-31a(X^{1a}═Me)} |
| H | N(Me)(D1-32a) |
| H | N(Me)(D1-33a) |
| H | N(Me)(D1-34a) |
| H | N(Me)(D1-35a) |
| H | N(Me)(D1-35b) |
| H | N(Me)(D1-36a) |
| H | N(Me)(D1-37a) |
| H | N(Me)(D1-38a) |
| H | N(Me)(D1-39a) |
| H | N(Me)(D1-40a) |
| H | N(Me)(D1-40b) |
| H | N(Me)(D1-40c) |
| H | N(Me)(D1-41a) |
| H | N(Me)(D1-42a) |
| H | N(Me)(D1-43a) |
| H | N(Me)(D1-45a) |
| H | N(Me)(D1-50a) |
| H | N(Me)(D1-51a) |
| H | N(Me)(D1-59a) |
| H | N(Me)(D1-61a) |
| H | N(Me)(D1-79a) |
| H | N(Me)(D1-80a) |
| H | N(Me){D1-108b(2-Cl)} |
| H | N(Me){D1-108b(3-Cl)} |
| H | N(Me){D1-108b(4-Cl)} |
| H | N(Me){D1-108b(2-Me)} |
| H | N(Me){D1-108b(3-Me)} |
| H | N(Me){D1-108b(4-Me)} |
| H | D1-8c(CH₂CF₃) |
| H | D1-1b(5-Br) |
| H | D1-2c(5-SMe) |
| H | D1-2c{5-C(O)Me} |
| H | D1-4a(Me) |
| H | D1-5d{5-(Pr-c)} |
| H | D1-5e(3,5-Me₂) |
| H | D1-6d(4,5-Cl₂) |
| H | D1-9b |
| H | D1-10e(2-CF₃) |
| H | D1-10f{2-(CH₂SCH₃)-4-CH₃} |
| H | D1-12f(X^{1a}═Me, X¹═4-NO₂) |
| H | D1-13d(Me) |
| H | D1-19a |
| H | D1-32b(2-Br) |
| H | D1-33b(2-OMe) |
| H | D1-34a |
| H | D1-35b |
| H | D1-38b(4-SMe) |
| H | D1-39b(5-Me) |
| H | D1-45d(5,5-Me₂) |
| H | D1-82a |
| H | D1-82b |
| H | D1-82c |
| H | D1-87a |
| H | D1-88a{X^{1a}═C(O)Me} |
| H | D1-88a{X^{1a}═C(O)OBu-t} |
| H | D1-103b |
| H | D1-103c |
| H | D1-103d |
| H | D1-103e |
| H | CH₂(D1-2a) |
| H | CH₂{D1-5f(3-Me)} |
| H | CH₂{D1-8f(X^{1a}═Me, X¹═3,5-Me₂)} |
| H | CH₂{D1-8f(X^{1a}═Me, X¹═3,5-Cl₂)} |
| H | CH₂{D1-9e(2-Ph, 5-Me)} |
| H | CH₂(D1-28a) |
| H | CH₂(D1-32a) |
| H | CH₂(D1-33a) |
| H | CH₂(D1-34a) |
| H | CH₂{D1-33b(6-Cl)} |
| H | CH₂(D1-34a) |
| H | CH₂CH₂{D1-7b(3-CF₃, 5-Me) |
| H | CH₂CH₂{D1-7b(3-CF₃, 5-Pr-c) |
| Me | C(O)Me |
| Me | Me |
| Me | Et |
| Me | Pr-n |
| Me | Pr-i |
| Me | Pr-c |
| Me | Bu-n |
| Me | Bu-i |
| Me | Bu-c |
| Me | Bu-s |
| Me | Bu-t |
| Me | Pen-n |
| Me | Pen-c |
| Me | Hex-n |
| Me | Hex-c |
| Me | CH═CH₂ |
| Me | C≡CH |
| Me | C≡CSiMe₃ |
| Me | CH₂Br |
| Me | CH₂Br |
| Me | CH(Br)Me |
| Me | CH₂CF₃ |
| Me | CH₂CH(OH)CF₃ |
| Me | CH₂CN |
| Me | CH(Me)CN |
| Me | CH₂CH₂CN |
| Me | CH(Me)CH₂CN |
| Me | CH₂CH₂CH₂CN |
| Me | CH(Me)CH₂CH₂CN |
| Me | CH₂CH₂CH₂CH₂CN |
| Me | CH(Me)CH₂CH₂CH₂CN |
| Me | CH₂C(O)OMe |
| Me | CH(Me)C(O)OMe |
| Me | CH₂CH₂C(O)OMe |
| Me | CH(Me)CH₂C(O)OMe |
| Me | CH₂CH₂CH₂C(O)OMe |
| Me | CH(Me)CH₂CH₂C(O)OMe |
| Me | CH₂CH₂CH₂CH₂C(O)OMe |
| Me | CH₂C(O)OEt |
| Me | CH(Me)C(O)OEt |
| Me | CH₂CH₂C(O)OEt |
| Me | CH(Me)CH₂C(O)OEt |
| Me | CH₂CH₂CH₂C(O)OEt |
| Me | CH(Me)CH₂CH₂C(O)OEt |
| Me | CH₂CH₂CH₂CH₂C(O)OEt |
| Me | CH₂C(O)NHMe |
| Me | CH₂OMe |
| Me | CH₂OEt |
| Me | CH₂OPr-n |
| Me | CH₂OPr-i |
| Me | CH₂OPr-c |
| Me | CH₂OBu-n |
| Me | CH₂OBu-i |
| Me | CH₂OBu-s |
| Me | CH₂OBu-t |
| Me | CH₂OBu-c |
| Me | CH₂OPen-n |
| Me | CH₂OPen-c |
| Me | CH₂OHex-n |
| Me | CH₂OHex-c |
| Me | CH₂OCH₂Pr-c |
| Me | CH₂OCH₂CH═CH₂ |
| Me | CH₂OCH₂C≡CH |
| Me | CH₂OCH₂OMe |
| Me | CH₂OCH₂SMe |
| Me | CH₂OCH₂Si(CH₃)₃ |
| Me | CH₂OCH₂(D1-34a) |
| Me | CH₂OCF₃ |
| Me | CH₂OCF₂H |
| Me | CH₂OCF₂Cl |
| Me | CH₂OCF₂Br |
| Me | CH₂OCH₂CF₃ |
| Me | CH₂OCH₂CF₂CF₃ |
| Me | CH₂OCH₂CN |
| Me | CH₂OC(O)Me |
| Me | CH₂OCH₂C(O)OMe |
| Me | CH₂OCH₂C(O)OEt |
| Me | CH₂ON═CHMe |
| Me | CH₂ON═C(Me)₂ |
| Me | CH(Me)OMe |
| Me | CH(Me)OEt |
| Me | CH(Me)OPr-n |
| Me | CH(Me)OPr-i |
| Me | CH(Me)OPr-c |
| Me | CH(Me)OBu-n |
| Me | CH(Me)OBu-i |
| Me | CH(Me)OBu-s |
| Me | CH(Me)OBu-t |
| Me | CH(Me)OBu-c |
| Me | CH(Me)OPen-n |
| Me | CH(Me)OPen-c |
| Me | CH(Me)OHex-n |
| Me | CH(Me)OHex-c |
| Me | CH(Me)OCH₂Pr-c |
| Me | CH(Me)OCH₂CH═CH₂ |
| Me | CH(Me)OCH₂C≡CH |
| Me | CH(Me)OCH₂OMe |
| Me | CH(Me)OCH₂SMe |
| Me | CH(Me)OCH₂Si(CH₃)₃ |
| Me | CH(Me)OCH₂(D1-34a) |
| Me | CH(Me)OCF₃ |
| Me | CH(Me)OCF₂H |
| Me | CH(Me)OCF₂Cl |
| Me | CH(Me)OCF₂Br |
| Me | CH(Me)OCH₂CF₃ |
| Me | CH(Me)OCH₂CF₂CF₃ |
| Me | CH(Me)OCH₂CN |
| Me | CH(Me)OC(O)Me |
| Me | CH(Me)OCH₂C(O)OMe |
| Me | CH(Me)OCH₂C(O)OEt |
| Me | CH(Me)ON═CHMe |
| Me | CH(Me)ON═C(Me)₂ |
| Me | CH₂SMe |
| Me | CH₂S(O)Me |
| Me | CH₂S(O)₂Me |
| Me | CH₂SEt |
| Me | CH₂S(O)Et |
| Me | CH₂S(O)₂Et |
| Me | CH₂SCH₂CH═CH₂ |
| Me | CH₂S(O)CH₂CH═CH₂ |
| Me | CH₂S(O)₂CH₂CH═CH₂ |
| Me | CH₂SCH₂CF₃ |
| Me | CH₂S(O)CH₂CF₃ |
| Me | CH₂S(O)₂CH₂CF₃ |
| Me | CH(Me)SMe |
| Me | CH(Me)S(O)Me |
| Me | CH(Me)S(O)₂Me |
| Me | CH(Me)SEt |
| Me | CH(Me)S(O)Et |
| Me | CH(Me)S(O)₂Et |
| Me | CH(Me)S(═NCN)Et |
| Me | CH(Me)S(O)(═NCN)Et |
| Me | CH(Me)SPr-n |
| Me | CH(Me)S(O)Pr-n |
| Me | CH(Me)S(O)₂Pr-n |
| Me | CH(Me)SPr-i |
| Me | CH(Me)S(O)Pr-i |
| Me | CH(Me)S(O)₂Pr-i |
| Me | CH(Me)SBu-t |
| Me | CH(Me)S(O)Bu-t |
| Me | CH(Me)S(O)₂Bu-t |
| Me | CH(Me)SCH₂Pr-c |
| Me | CH(Me)S(O)CH₂Pr-c |
| Me | CH(Me)S(O)₂CH₂Pr-c |
| Me | CH(Me)SCH₂OMe |
| Me | CH(Me)SCH₂SMe |
| Me | CH(Me)S(O)CH₂SMe |
| Me | CH(Me)S(O)₂CH₂SMe |
| Me | CH(Me)SCH₂C≡CH |
| Me | CH(Me)S(O)CH₂C≡CH |
| Me | CH(Me)S(O)₂CH₂C≡CH |
| Me | CH(Me)SCH₂C(O)NHMe |
| Me | CH(Me)S(O)CH₂C(O)NHMe |
| Me | CH(Me)S(O)₂CH₂C(O)NHMe |
| Me | CH(Me)SCH₂C(O)OMe |
| Me | CH(Me)S(O)CH₂C(O)OMe |
| Me | CH(Me)S(O)₂CH₂C(O)OMe |
| Me | CH(Me)SCH₂CF₃ |
| Me | CH(Me)S(O)CH₂CF₃ |
| Me | CH(Me)S(O)₂CH₂CF₃ |
| Me | CH(Me)SCH₂(D1-34a) |
| Me | CH(Me)S(O)CH₂(D1-34a) |
| Me | CH(Me)S(O)₂CH₂(D1-34a) |
| Me | CH(CH₃)SCH₂Si(CH₃)₃ |
| Me | CH(Me)SCN |
| Me | CH(Me)SCH₂CN |
| Me | CH(Me)SC(O)Me |
| Me | CH(Me)S(D1-37a) |
| Me | CH(Et)SMe |
| Me | CH(Et)S(O)Me |
| Me | CH(Et)S(O)₂Me |
| Me | CH(Et)SEt |
| Me | CH(Et)S(O)Et |
| Me | CH(Et)S(O)₂Et |
| Me | CH(Et)SCH₂CF₃ |
| Me | CH(Et)S(O)CH₂CF₃ |
| Me | CH(Et)S(O)₂CH₂CF₃ |
| Me | CH(F)SMe |
| Me | CH(F)S(O)Me |
| Me | CH(F)S(O)₂Me |
| Me | CH(F)SEt |
| Me | CH(F)S(O)Et |
| Me | CH(F)S(O)₂Et |
| Me | CH{OC(O)Me}SMe |
| Me | CH{OC(O)Me}S(O)Me |
| Me | CH{OC(O)Me}S(O)₂Me |
| Me | CH(SMe)₂ |
| Me | C(Me)₂SMe |
| Me | C(Me)₂S(O)Me |
| Me | C(Me)₂S(O)₂Me |
| Me | CH₂CH₂SH |
| Me | CH₂CH₂SMe |
| Me | CH₂CH₂S(O)Me |
| Me | CH₂CH₂S(O)₂Me |
| Me | CH₂CH₂SEt |
| Me | CH₂CH₂S(O)Et |
| Me | CH₂CH₂S(O)₂Et |
| Me | CH₂CH₂SPr-n |
| Me | CH₂CH₂S(O)Pr-n |
| Me | CH₂CH₂S(O)₂Pr-n |
| Me | CH₂CH₂SPr-i |
| Me | CH₂CH₂S(O)Pr-i |
| Me | CH₂CH₂S(O)₂Pr-i |
| Me | CH(Me)SCH₂CH₂CH₂Cl |
| Me | CH(Me)S(O)CH₂CH₂CH₂Cl |
| Me | CH(Me)S(O)₂CH₂CH₂CH₂Cl |
| Me | CH₂CH₂SCH₂CF₃ |
| Me | CH₂CH₂S(O)CH₂CF₃ |
| Me | CH₂CH₂S(O)₂CH₂CF₃ |
| Me | CH₂CH₂SC(O)Me |
| Me | CH(Me)CH₂SMe |
| Me | CH(Me)CH₂S(O)Me |
| Me | CH(Me)CH₂S(O)₂Me |
| Me | CH(Me)CH₂SEt |
| Me | CH(Me)CH₂S(O)Et |
| Me | CH(Me)CH₂S(O)₂Et |
| Me | CH(Me)CH₂SPr-n |
| Me | CH(Me)CH₂S(O)Pr-n |
| Me | CH(Me)CH₂S(O)₂Pr-n |
| Me | CH(Me)CH₂SPr-i |
| Me | CH(Me)CH₂S(O)Pr-i |
| Me | CH(Me)CH₂S(O)₂Pr-i |
| Me | CH(Me)CH₂SCH₂CF₃ |
| Me | CH(Me)CH₂S(O)CH₂CF₃ |
| Me | CH(Me)CH₂S(O)₂CH₂CF₃ |
| Me | CH(Me)CH₂SC(O)Me |
| Me | CH(Et)CH₂SMe |
| Me | CH(Et)CH₂S(O)Me |
| Me | CH(Et)CH₂S(O)₂Me |
| Me | CH(OMe)CH₂SMe |
| Me | CH(OMe)CH₂S(O)Me |
| Me | CH(OMe)CH₂S(O)₂Me |
| Me | CH(SMe)CH₂SMe |
| Me | CH(SMe)CH₂S(O)Me |
| Me | CH(SMe)CH₂S(O)₂Me |
| Me | CH(CH₂SMe)CH₂SMe |
| Me | CH(CH₂SMe)CH₂S(O)Me |
| Me | CH(CH₂SMe)CH₂S(O)₂Me |
| Me | CH(Cl)CH₂SMe |
| Me | CH(Cl)CH₂S(O)Me |
| Me | CH(Cl)CH₂S(O)₂Me |
| Me | CH(CN)CH₂SMe |
| Me | CH(CN)CH₂S(O)Me |
| Me | CH(CN)CH₂S(O)₂Me |
| Me | CH(CN)CH₂SEt |
| Me | CH(CN)CH₂S(O)Et |
| Me | CH(CN)CH₂S(O)₂Et |
| Me | CH{C(O)OMe}CH₂SMe |
| Me | CH{C(O)OMe}CH₂S(O)Me |
| Me | CH{C(O)OMe}CH₂S(O)₂Me |
| Me | CH{C(O)OMe}CH₂SEt |
| Me | CH{C(O)OMe}CH₂S(O)Et |
| Me | CH{C(O)OMe}CH₂S(O)₂Et |
| Me | CH(Ph)CH₂SMe |
| Me | CH(Ph)CH₂S(O)Me |
| Me | CH(Ph)CH₂S(O)₂Me |
| Me | C(Me)₂CH₂SMe |
| Me | C(Me)₂CH₂S(O)Me |
| Me | C(Me)₂CH₂S(O)₂Me |
| Me | C(═CH₂)CH₂SMe |
| Me | C(═CH₂)CH₂S(O)Me |
| Me | C(═CH₂)CH₂S(O)₂Me |
| Me | C(═CH₂)CH₂SEt |
| Me | C(═CH₂)CH₂S(O)Et |
| Me | C(═CH₂)CH₂S(O)₂Et |
| Me | CH₂CH₂CH₂SEt |
| Me | CH₂CH₂CH₂S(O)Et |
| Me | CH₂CH₂CH₂S(O)₂Et |
| Me | CH(Me)CH₂CH₂SEt |
| Me | CH(Me)CH₂CH₂S(O)Et |
| Me | CH(Me)CH₂CH₂S(O)₂Et |
| Me | CH₂CH(Me)CH₂SEt |
| Me | CH₂CH(Me)CH₂S(O)Et |
| Me | CH₂CH(Me)CH₂S(O)₂Et |
| Me | CH₂CH₂CH(Me)SEt |
| Me | CH₂CH₂CH(Me)S(O)Et |
| Me | CH₂CH₂CH(Me)S(O)₂Et |
| Me | CH₂CH₂CH₂SPr-n |
| Me | CH₂CH₂CH₂S(O)Pr-n |
| Me | CH₂CH₂CH₂S(O)₂Pr-n |
| Me | CH(Me)CH₂CH₂SPr-n |
| Me | CH(Me)CH₂CH₂S(O)Pr-n |
| Me | CH(Me)CH₂CH₂S(O)₂Pr-n |
| Me | CH₂CH(Me)CH₂SPr-n |
| Me | CH₂CH(Me)CH₂S(O)Pr-n |
| Me | CH₂CH(Me)CH₂S(O)₂Pr-n |
| Me | CH₂CH₂CH₂CH₂SMe |
| Me | CH₂CH₂CH₂CH₂S(O)Me |
| Me | CH₂CH₂CH₂CH₂S(O)₂Me |
| Me | CH₂CH₂CH₂CH₂SEt |
| Me | CH₂CH₂CH₂CH₂S(O)Et |
| Me | CH₂CH₂CH₂CH₂S(O)₂Et |
| Me | CH₂CH₂CH₂CH₂SPr-n |
| Me | CH₂CH₂CH₂CH₂S(O)Pr-n |
| Me | CH₂CH₂CH₂CH₂S(O)₂Pr-n |
| Me | CH₂CH(Me)SMe |
| Me | CH₂OCH₂SMe |
| Me | CH═CHSMe |
| Me | CH₂CH(SMe)₂ |
| Me | CH₂CH₂SC(O)Me |
| Me | C(═CH₂)CH₂C(O)OH |
| Me | C(═NOMe)CH₃ |
| Me | C(═NOMe)Et |
| Me | C(═NOMe)Pr-n |
| Me | C(═NOEt)CH₃ |
| Me | C(═NOPr-n)CH₃ |
| Me | C(═NOPr-i)CH₃ |
| Me | C(═NOBu-n)CH₃ |
| Me | C(═NOCH₂Ph)CH₃ |
| Me | C(═NOMe)CH₂OCH₂CF₃ |
| Me | C(═NOMe)CH₂SMe |
| Me | C(═NOMe)CH₂S(O)Me |
| Me | C(═NOMe)CH₂S(O)₂Me |
| Me | C(═NOMe)CH₂SEt |
| Me | C(═NOMe)CH₂S(O)Et |
| Me | C(═NOMe)CH₂S(O)₂Et |
| Me | C(═NOMe)CH₂SCF₃ |
| Me | C(═NOMe)CH₂S(O)CF₃ |
| Me | C(═NOMe)CH₂S(O)₂CF₃ |
| Me | C(═NOMe)CH₂SCH₂CF₃ |
| Me | C(═NOMe)CH₂S(O)CH₂CF₃ |
| Me | C(═NOMe)CH₂S(O)₂CH₂CF₃ |
| Me | C(═NOEt)CH₂SMe |
| Me | C(═NOEt)CH₂S(O)Me |
| Me | C(═NOEt)CH₂S(O)₂Me |
| Me | C(═NOPr-n)CH₂SMe |
| Me | C(═NOPr-n)CH₂S(O)Me |
| Me | C(═NOPr-n)CH₂S(O)₂Me |
| Me | C(═NOPr-i)CH₂SMe |
| Me | C(═NOPr-i)CH₂S(O)Me |
| Me | C(═NOPr-i)CH₂S(O)₂Me |
| Me | C(═NOBu-n)CH₂SMe |
| Me | C(═NOBu-n)CH₂S(O)Me |
| Me | C(═NOBu-n)CH₂S(O)₂Me |
| Me | C(═NOCH₂Ph)CH₂SMe |
| Me | C(═NOCH₂Ph)CH₂S(O)Me |
| Me | C(═NOCH₂Ph)CH₂S(O)₂Me |
| Me | C(═NOCH₂CH═CH₂)CH₂SMe |
| Me | C(═NOCH₂OMe)CH₂SMe |
| Me | C(═NOCH₂CH₂OMe)CH₂SMe |
| Me | C{═NOCH₂CH₂Si(Me)₃}CH₂SMe |
| Me | CH₂CH(═NOMe) |
| Me | CH₂C(═NOMe)Me |
| Me | CH₂C(═NOMe)Et |
| Me | CH₂C(═NOMe)Pr-n |
| Me | CH₂C(═NOMe)Pr-i |
| Me | CH₂C(═NOMe)Bu-n |
| Me | CH₂C(═NOMe)Bu-i |
| Me | CH₂C(═NOMe)Bu-s |
| Me | CH₂C(═NOMe)Bu-t |
| Me | CH₂C(═NOMe)CF₃ |
| Me | CH₂C(═NOCH₃)CH₂SCH₃ |
| Me | CH(SMe)C(═NOMe)Me |
| Me | CH{S(O)Me}C(═NOMe)Me |
| Me | CH{S(O)₂Me}C(═NOMe)Me |
| Me | CH(SEt)C(═NOMe)Me |
| Me | CH{S(O)Et}C(═NOMe)Me |
| Me | CH{S(O)₂Et}C(═NOMe)Me |
| Me | CH₂NH₂ |
| Me | CH₂NHC(O)Me |
| Me | CH₂NHC(O)OMe |
| Me | CH₂NHC(O)OBu-t |
| Me | CH₂NHSO₂Me |
| Me | CH₂N(Me)C(O)Me |
| Me | CH₂N(Me)C(O)OMe |
| Me | CH₂N(Me)C(O)OBu-t |
| Me | CH₂N(Me)SO₂Me |
| Me | CH(CH₂SMe)NH₂ |
| Me | CH(CH₂SMe)NHC(O)Me |
| Me | CH(CH₂SMe)NHC(O)OMe |
| Me | CH(CH₂SMe)NHC(O)OBu-t |
| Me | CH(CH₂SMe)NHSO₂Me |
| Me | CH₂P(O)(OMe)₂ |
| Me | CH₂P(O)(OEt)₂ |
| Me | OBu-t |
| Me | SMe |
| Me | NH₂ |
| Me | NHCH₂CH₂SMe |
| Me | N(Me)CH₂CH₂SMe |
| Me | NHCH₂CH₂CH₂SMe |
| Me | NHMe |
| Me | NHEt |
| Me | NPr-n |
| Me | NHCH(Me)₂ |
| Me | NHCH₂C(O)OMe |
| Me | NHCH₂CF₃ |
| Me | N(Me)CH₂CF₃ |
| Me | NHC(O)OEt |
| Me | NHC(O)CCl₃ |
| Me | NHC(O)CH₂SMe |
| Me | N(CH₂CH₃)C(O)CH₂SCH₃ |
| Me | NHS(O)₂(D1-108a) |
| Me | NHPh |
| Me | NH(D1-1a) |
| Me | NH(D1-2a) |
| Me | NH(D1-2b) |
| Me | NH{D1-4a(X^{1a}═Me)} |
| Me | NH{D1-4b(X^{1a}═Me)} |
| Me | NH(D1-5a) |
| Me | NH(D1-5b) |
| Me | NH(D1-5c) |
| Me | NH(D1-6a) |
| Me | NH(D1-6b) |
| Me | NH(D1-6c) |
| Me | NH(D1-7a) |
| Me | NH{D1-8a(X^{1a}═Me)} |
| Me | NH{D1-8b(X^{1a}═Me)} |
| Me | NH{D1-8c(X^{1a}═Me)} |
| Me | NH(D1-9a) |
| Me | NH(D1-9b) |
| Me | NH(D1-9c) |
| Me | NH(D1-10a) |
| Me | NH(D1-10b) |
| Me | NH(D1-10c) |
| Me | NH(D1-11a) |
| Me | NH{D1-12a(X^{1a}═Me)} |
| Me | NH{D1-12b(X^{1a}═Me)} |
| Me | NH{D1-12c(X^{1a}═Me)} |
| Me | NH(D1-13a) |
| Me | NH(D1-13b) |
| Me | NH(D1-14a) |
| Me | NH(D1-14b) |
| Me | NH(D1-15a) |
| Me | NH(D1-15b) |
| Me | NH(D1-16a) |
| Me | NH(D1-17a) |
| Me | NH(D1-18a) |
| Me | NH(D1-19a) |
| Me | NH(D1-20a) |
| Me | NH{D1-21a(X^{1a}═Me)} |
| Me | NH{D1-21b(X^{1a}═Me)} |
| Me | NH(D1-22a) |
| Me | NH{D1-23a(X^{1a}═Me)} |
| Me | NH{D1-23b(X^{1a}═Me)} |
| Me | NH(D1-24a) |
| Me | NH{D1-25a(X^{1a}═Me)} |
| Me | NH(D1-26a) |
| Me | NH{D1-27a(X^{1a}═Me)} |
| Me | NH(D1-28a) |
| Me | NH(D1-29a) |
| Me | NH{D1-30a(X^{1a}═Me)} |
| Me | NH{D1-31a(X^{1a}═Me)} |
| Me | NH(D1-32a) |
| Me | NH(D1-33a) |
| Me | NH(D1-34a) |
| Me | NH(D1-35a) |
| Me | NH(D1-35b) |
| Me | NH(D1-36a) |
| Me | NH(D1-37a) |
| Me | NH(D1-38a) |
| Me | NH(D1-39a) |
| Me | NH(D1-40a) |
| Me | NH(D1-40b) |
| Me | NH(D1-40c) |
| Me | NH(D1-41a) |
| Me | NH(D1-42a) |
| Me | NH(D1-43a) |
| Me | NH(D1-45a) |
| Me | NH(D1-50a) |
| Me | NH(D1-51a) |
| Me | NH(D1-59a) |
| Me | NH(D1-61a) |
| Me | NH(D1-79a) |
| Me | NH(D1-80a) |
| Me | NH{D1-108b(2-Cl)} |
| Me | NH{D1-108b(3-Cl)} |
| Me | NH{D1-108b(4-Cl)} |
| Me | NH{D1-108b(2-Me)} |
| Me | NH{D1-108b(3-Me)} |
| Me | NH{D1-108b(4-Me)} |
| Me | N(Me)Ph |
| Me | N(Me)(D1-1a) |
| Me | N(Me)(D1-2a) |
| Me | N(Me)(D1-2b) |
| Me | N(Me){D1-4a(X^{1a}═Me)} |
| Me | N(Me){D1-4b(X^{1a}═Me)} |
| Me | N(Me)(D1-5a) |
| Me | N(Me)(D1-5b) |
| Me | N(Me)(D1-5c) |
| Me | N(Me)(D1-6a) |
| Me | N(Me)(D1-6b) |
| Me | N(Me)(D1-6c) |
| Me | N(Me)(D1-7a) |
| Me | N(Me){D1-8a(X^{1a}═Me)} |
| Me | N(Me){D1-8b(X^{1a}═Me)} |
| Me | N(Me){D1-8c(X^{1a}═Me)} |
| Me | N(Me)(D1-9a) |
| Me | N(Me)(D1-9b) |
| Me | N(Me)(D1-9c) |
| Me | N(Me)(D1-10a) |
| Me | N(Me)(D1-10b) |
| Me | N(Me)(D1-10c) |
| Me | N(Me)(D1-11a) |
| Me | N(Me){D1-12a(X^{1a}═Me)} |
| Me | N(Me){D1-12b(X^{1a}═Me)} |
| Me | N(Me){D1-12c(X^{1a}═Me)} |
| Me | N(Me)(D1-13a) |
| Me | N(Me)(D1-13b) |
| Me | N(Me)(D1-14a) |
| Me | N(Me)(D1-14b) |
| Me | N(Me)(D1-15a) |
| Me | N(Me)(D1-15b) |
| Me | N(Me)(D1-16a) |
| Me | N(Me)(D1-17a) |
| Me | N(Me)(D1-18a) |
| Me | N(Me)(D1-19a) |
| Me | N(Me)(D1-20a) |
| Me | N(Me){D1-21a(X^{1a}═Me)} |
| Me | N(Me){D1-21b(X^{1a}═Me)} |
| Me | N(Me)(D1-22a) |
| Me | N(Me){D1-23a(X^{1a}═Me)} |
| Me | N(Me){D1-23b(X^{1a}═Me)} |
| Me | N(Me)(D1-24a) |
| Me | N(Me){D1-25a(X^{1a}═Me)} |
| Me | N(Me)(D1-26a) |
| Me | N(Me){D1-27a(X^{1a}═Me)} |
| Me | N(Me)(D1-28a) |
| Me | N(Me)(D1-29a) |
| Me | N(Me){D1-30a(X^{1a}═Me)} |
| Me | N(Me){D1-31a(X^{1a}═Me)} |
| Me | N(Me)(D1-32a) |
| Me | N(Me)(D1-33a) |
| Me | N(Me)(D1-34a) |
| Me | N(Me)(D1-35a) |
| Me | N(Me)(D1-35b) |
| Me | N(Me)(D1-36a) |
| Me | N(Me)(D1-37a) |
| Me | N(Me)(D1-38a) |
| Me | N(Me)(D1-39a) |
| Me | N(Me)(D1-40a) |
| Me | N(Me)(D1-40b) |
| Me | N(Me)(D1-40c) |
| Me | N(Me)(D1-41a) |
| Me | N(Me)(D1-42a) |
| Me | N(Me)(D1-43a) |
| Me | N(Me)(D1-45a) |
| Me | N(Me)(D1-50a) |
| Me | N(Me)(D1-51a) |
| Me | N(Me)(D1-59a) |
| Me | N(Me)(D1-61a) |
| Me | N(Me)(D1-79a) |
| Me | N(Me)(D1-80a) |
| Me | N(Me){D1-108b(2-Cl)} |
| Me | N(Me){D1-108b(3-Cl)} |
| Me | N(Me){D1-108b(4-Cl)} |
| Me | N(Me){D1-108b(2-Me)} |
| Me | N(Me){D1-108b(3-Me)} |
| Me | N(Me){D1-108b(4-Me)} |
| Me | D1-8c(CH₂CF₃) |
| Me | D1-1b(5-Br) |
| Me | D1-2c(5-SMe) |
| Me | D1-2c{5-C(O)Me} |
| Me | D1-4a(Me) |
| Me | D1-5d{5-(Pr-c)} |
| Me | D1-5e(3,5-Me₂) |
| Me | D1-6d(4,5-Cl₂) |
| Me | D1-9b |
| Me | D1-10e(2-CF₃) |
| Me | D1-10f{2-(CH₂SCH₃)-4-CH₃} |
| Me | D1-12f(X^{1a}═Me, X¹═4-NO₂) |
| Me | D1-13d(Me) |
| Me | D1-19a |
| Me | D1-32b(2-Br) |
| Me | D1-33b(2-OMe) |
| Me | D1-35b |
| Me | D1-38b(4-SMe) |
| Me | D1-39b(5-Me) |
| Me | D1-45d(5,5-Me₂) |
| Me | D1-82a |
| Me | D1-82b |
| Me | D1-82c |
| Me | D1-87a |
| Me | D1-88a{X^{1a}═C(O)Me} |
| Me | D1-88a{X^{1a}═C(O)OBu-t} |
| Me | D1-103b |
| Me | D1-103c |
| Me | D1-103d |
| Me | D1-103e |
| Me | CH₂(D1-2a) |
| Me | CH₂{D1-5f(3-Me)} |
| Me | CH₂{D1-8f(X^{1a}═Me, X¹═3,5-Me₂)} |
| Me | CH₂{D1-8f(X^{1a}═Me, X¹═3,5-Cl₂)} |
| Me | CH₂{D1-9e(2-Ph, 5-Me)} |
| Me | CH₂(D1-28a) |
| Me | CH₂(D1-32a) |
| Me | CH₂(D1-33a) |
| Me | CH₂{D1-33b(6-Cl)} |
| Me | CH₂(D1-34a) |
| Me | CH₂CH₂{D1-7b(3-CF₃, 5-Me) |
| Me | CH₂CH₂{D1-7b(3-CF₃, 5-Pr-c) |
| Et | C(O)(D1-2a) |
| Et | C(O)Me |
| Et | C(O)Et |
| Et | C(O)Pr-n |
| Et | C(O)Pr-i |
| Et | C(O)Pr-c |
| Et | C(O)Bu-n |
| Et | C(O)Bu-i |
| Et | C(O)Bu-s |
| Et | C(O)Bu-t |
| Et | C(O)Pen-n |
| Et | C(O)Pen-c |
| Et | C(O)Hex-n |
| Et | C(O)Hex-c |
| Et | C(O)CF₃ |
| Et | C(O)OMe |
| Et | C(O)OEt |
| Et | C(O)NHPh |
| Et | Me |
| Et | Et |
| Et | Pr-n |
| Et | Pr-i |
| Et | Pr-c |
| Et | Bu-n |
| Et | Bu-i |
| Et | Bu-c |
| Et | Bu-s |
| Et | Bu-t |
| Et | Pen-n |
| Et | Pen-c |
| Et | Hex-n |
| Et | Hex-c |
| Et | (CH₂)₆CH₃ |
| Et | (CH₂)₇CH₃ |
| Et | (CH₂)₈CH₃ |
| Et | (CH₂)₉CH₃ |
| Et | (CH₂)₁₀CH₃ |
| Et | (CH₂)₁₁CH₃ |
| Et | CH(Me)(CH₂)₂CH₃ |
| Et | CH(Me)(CH₂)₃CH₃ |
| Et | CH(Me)(CH₂)₄CH₃ |
| Et | CH(Me)(CH₂)₅CH₃ |
| Et | CH(Me)(CH₂)₆CH₃ |
| Et | CH(Me)(CH₂)₇CH₃ |
| Et | CH(Me)(CH₂)₈CH₃ |
| Et | CH₂Pr-c |
| Et | CH₂Bu-t |
| Et | CH═CH₂ |
| Et | C(Me)═CH₂ |
| Et | CH═CH(D1-1a) |
| Et | CH═CH(D1-2a) |
| Et | CH═CH(D1-33a) |
| Et | CH═CH{D1-108b(4-CF₃)} |
| Et | C≡CH |
| Et | C≡CSiMe₃ |
| Et | CHCl₂ |
| Et | CH₂Br |
| Et | CH(Br)Me |
| Et | CH₂CH₂CH₂CH₂Br |
| Et | CH(Br)Bu-t |
| Et | CH₂CF₃ |
| Et | CF₂CF₂Cl |
| Et | CH₂CH(CF₃)₂ |
| Et | CH₂CH(OH)CF₃ |
| Et | CH₂CH₂C(F)═CF₂ |
| Et | CH₂CN |
| Et | CH(Me)CN |
| Et | CH₂CH₂CN |
| Et | CH(Me)CH₂CN |
| Et | CH₂CH₂CH₂CN |
| Et | CH(Me)CH₂CH₂CN |
| Et | CH₂CH₂CH₂CH₂CN |
| Et | CH(Me)CH₂CH₂CH₂CN |
| Et | CH₂C(O)OMe |
| Et | CH(Me)C(O)OMe |
| Et | CH₂CH₂C(O)OMe |
| Et | CH(Me)CH₂C(O)OMe |
| Et | CH₂CH₂CH₂C(O)OMe |
| Et | CH(Me)CH₂CH₂C(O)OMe |
| Et | CH₂CH₂CH₂CH₂C(O)OMe |
| Et | CH₂C(O)OEt |
| Et | CH(Me)C(O)OEt |
| Et | CH₂CH₂C(O)OEt |
| Et | CH(Me)CH₂C(O)OEt |
| Et | CH₂CH₂CH₂C(O)OEt |
| Et | CH(Me)CH₂CH₂C(O)OEt |
| Et | CH₂CH₂CH₂CH₂C(O)OEt |
| Et | CH₂C(O)NHMe |
| Et | CH₂C(O)NHCH₂CF₃ |
| Et | CH₂OMe |
| Et | CH₂OEt |
| Et | CH₂OPr-n |
| Et | CH₂OPr-i |
| Et | CH₂OPr-c |
| Et | CH₂OBu-n |
| Et | CH₂OBu-i |
| Et | CH₂OBu-s |
| Et | CH₂OBu-t |
| Et | CH₂OBu-c |
| Et | CH₂OPen-n |
| Et | CH₂OPen-c |
| Et | CH₂OHex-n |
| Et | CH₂OHex-c |
| Et | CH₂OCH₂Pr-c |
| Et | CH₂OCH₂CH═CH₂ |
| Et | CH₂OCH₂C≡CH |
| Et | CH₂OCH₂OMe |
| Et | CH₂OCH₂SMe |
| Et | CH₂OCH₂Si(CH₃)₃ |
| Et | CH₂OCH₂(D1-34a) |
| Et | CH₂OCF₃ |
| Et | CH₂OCF₂H |
| Et | CH₂OCF₂Cl |
| Et | CH₂OCF₂Br |
| Et | CH₂OCH₂CF₃ |
| Et | CH₂OCH₂CF₂CF₃ |
| Et | CH₂OCH₂CN |
| Et | CH₂OC(O)Me |
| Et | CH₂OCH₂C(O)OMe |
| Et | CH₂OCH₂C(O)OEt |
| Et | CH₂ON═CHMe |
| Et | CH₂ON═C(Me)₂ |
| Et | CH(Me)OMe |
| Et | CH(Me)OEt |
| Et | CH(Me)OPr-n |
| Et | CH(Me)OPr-i |
| Et | CH(Me)OPr-c |
| Et | CH(Me)OBu-n |
| Et | CH(Me)OBu-i |
| Et | CH(Me)OBu-s |
| Et | CH(Me)OBu-t |
| Et | CH(Me)OBu-c |
| Et | CH(Me)OPen-n |
| Et | CH(Me)OPen-c |
| Et | CH(Me)OHex-n |
| Et | CH(Me)OHex-c |
| Et | CH(Me)OCH₂Pr-c |
| Et | CH(Me)OCH₂CH═CH₂ |
| Et | CH(Me)OCH₂C≡CH |
| Et | CH(Me)OCH₂OMe |
| Et | CH(Me)OCH₂SMe |
| Et | CH(Me)OCH₂Si(CH₃)₃ |
| Et | CH(Me)OCH₂(D1-34a) |
| Et | CH(Me)OCF₃ |
| Et | CH(Me)OCF₂H |
| Et | CH(Me)OCF₂Cl |
| Et | CH(Me)OCF₂Br |
| Et | CH(Me)OCH₂CF₃ |
| Et | CH(Me)OCH₂CF₂CF₃ |
| Et | CH(Me)OCH₂CN |
| Et | CH(Me)OC(O)Me |
| Et | CH(Me)OCH₂C(O)OMe |
| Et | CH(Me)OCH₂C(O)OEt |
| Et | CH(Me)ON═CHMe |
| Et | CH(Me)ON═C(Me)₂ |
| Et | C(Me)₂OC(O)Me |
| Et | CH₂SMe |
| Et | CH₂S(O)Me |
| Et | CH₂S(O)₂Me |
| Et | CH₂SEt |
| Et | CH₂S(O)Et |
| Et | CH₂S(O)₂Et |
| Et | CH₂SPr-n |
| Et | CH₂S(O)Pr-n |
| Et | CH₂S(O)₂Pr-n |
| Et | CH₂SPr-i |
| Et | CH₂S(O)Pr-i |
| Et | CH₂S(O)₂Pr-i |
| Et | CH₂SPr-c |
| Et | CH₂S(O)Pr-c |
| Et | CH₂S(O)₂Pr-c |
| Et | CH₂SCH₂CH═CH₂ |
| Et | CH₂S(O)CH₂CH═CH₂ |
| Et | CH₂S(O)₂CH₂CH═CH₂ |
| Et | CH₂SCH₂C≡CH |
| Et | CH₂S(O)CH₂C≡CH |
| Et | CH₂S(O)₂CH₂C≡CH |
| Et | CH₂SCH₂CF₃ |
| Et | CH₂S(O)CH₂CF₃ |
| Et | CH₂S(O)₂CH₂CF₃ |
| Et | CH₂SCH₂CH(OMe)₂ |
| Et | CH₂SCH₂CH(═NOMe) |
| Et | CH(Me)SMe |
| Et | CH(Me)S(O)Me |
| Et | CH(Me)S(O)₂Me |
| Et | CH(Me)SEt |
| Et | CH(Me)S(O)Et |
| Et | CH(Me)S(O)₂Et |
| Et | CH(Me)S(═NCN)Et |
| Et | CH(Me)S(O)(═NCN)Et |
| Et | CH(Me)SPr-n |
| Et | CH(Me)S(O)Pr-n |
| Et | CH(Me)S(O)₂Pr-n |
| Et | CH(Me)SPr-i |
| Et | CH(Me)S(O)Pr-i |
| Et | CH(Me)S(O)₂Pr-i |
| Et | CH(Me)SBu-t |
| Et | CH(Me)S(O)Bu-t |
| Et | CH(Me)S(O)₂Bu-t |
| Et | CH(Me)SCH₂Pr-c |
| Et | CH(Me)S(O)CH₂Pr-c |
| Et | CH(Me)S(O)₂CH₂Pr-c |
| Et | CH(Me)SCH₂OMe |
| Et | CH(Me)SCH₂SMe |
| Et | CH(Me)S(O)CH₂SMe |
| Et | CH(Me)S(O)₂CH₂SMe |
| Et | CH(Me)SCH₂C═CH₂ |
| Et | CH(Me)S(O)CH₂C═CH₂ |
| Et | CH(Me)S(O)₂CH₂C═CH₂ |
| Et | CH(Me)SCH₂C≡CH |
| Et | CH(Me)S(O)CH₂C≡CH |
| Et | CH(Me)S(O)₂CH₂C≡CH |
| Et | CH(Me)SCH₂C(O)NHMe |
| Et | CH(Me)S(O)CH₂C(O)NHMe |
| Et | CH(Me)S(O)₂CH₂C(O)NHMe |
| Et | CH(Me)SCH₂C(O)OMe |
| Et | CH(Me)S(O)CH₂C(O)OMe |
| Et | CH(Me)S(O)₂CH₂C(O)OMe |
| Et | CH(Me)SCH₂CH₂CH₂Cl |
| Et | CH(Me)S(O)CH₂CH₂CH₂Cl |
| Et | CH(Me)S(O)₂CH₂CH₂CH₂Cl |
| Et | CH(Me)SCH₂CF₃ |
| Et | CH(Me)S(O)CH₂CF₃ |
| Et | CH(Me)S(O)₂CH₂CF₃ |
| Et | CH(Me)SCH₂Ph |
| Et | CH(Me)S(O)CH₂Ph |
| Et | CH(Me)S(O)₂CH₂Ph |
| Et | CH(Me)SCH₂(D1-34a) |
| Et | CH(Me)S(O)CH₂(D1-34a) |
| Et | CH(Me)S(O)₂CH₂(D1-34a) |
| Et | CH(CH₃)SCH₂Si(CH₃)₃ |
| Et | CH(Me)SCN |
| Et | CH(Me)SCH₂CN |
| Et | CH(Me)SC(O)Me |
| Et | CH(Me)S{D1-12a(X^{1a}═Me)} |
| Et | CH(Me)S(D1-32a) |
| Et | CH(Me)S{D1-32b(3-NO₂)} |
| Et | CH(Me)S{D1-32b(3-CF₃)} |
| Et | CH(Me)S(D1-37a) |
| Et | CH(Me)S(D1-51a) |
| Et | CH(Et)SMe |
| Et | CH(Et)S(O)Me |
| Et | CH(Et)S(O)₂Me |
| Et | CH(Et)SEt |
| Et | CH(Et)S(O)Et |
| Et | CH(Et)S(O)₂Et |
| Et | CH(Et)S(═NCN)Et |
| Et | CH(Et)S(O)(═NCN)Et |
| Et | CH(Et)SPr-n |
| Et | CH(Et)S(O)Pr-n |
| Et | CH(Et)S(O)₂Pr-n |
| Et | CH(Et)SPr-i |
| Et | CH(Et)S(O)Pr-i |
| Et | CH(Et)S(O)₂Pr-i |
| Et | CH(Et)SPr-c |
| Et | CH(Et)S(O)Pr-c |
| Et | CH(Et)S(O)₂Pr-c |
| Et | CH(Et)SCH₂CF₃ |
| Et | CH(Et)S(O)CH₂CF₃ |
| Et | CH(Et)S(O)₂CH₂CF₃ |
| Et | CH(Pr-n)SMe |
| Et | CH(Pr-n)S(O)Me |
| Et | CH(Pr-n)S(O)₂Me |
| Et | CH(Pr-n)SEt |
| Et | CH(Pr-n)S(O)Et |
| Et | CH(Pr-n)S(O)₂Et |
| Et | CH(Pr-n)S(═NCN)Et |
| Et | CH(Pr-n)S(O)(═NCN)Et |
| Et | CH(Pr-n)SPr-n |
| Et | CH(Pr-n)S(O)Pr-n |
| Et | CH(Pr-n)S(O)₂Pr-n |
| Et | CH(Pr-n)SPr-i |
| Et | CH(Pr-n)S(O)Pr-i |
| Et | CH(Pr-n)S(O)₂Pr-i |
| Et | CH(Pr-n)SPr-c |
| Et | CH(Pr-n)S(O)Pr-c |
| Et | CH(Pr-n)S(O)₂Pr-c |
| Et | CH(Pr-n)SCH₂CF₃ |
| Et | CH(Pr-n)S(O)CH₂CF₃ |
| Et | CH(Pr-n)S(O)₂CH₂CF₃ |
| Et | CH(Pr-i)SMe |
| Et | CH(Pr-i)S(O)Me |
| Et | CH(Pr-i)S(O)₂Me |
| Et | CH(Pr-i)SEt |
| Et | CH(Pr-i)S(O)Et |
| Et | CH(Pr-i)S(O)₂Et |
| Et | CH(Pr-i)S(═NCN)Et |
| Et | CH(Pr-i)S(O)(═NCN)Et |
| Et | CH(Pr-i)SPr-n |
| Et | CH(Pr-i)S(O)Pr-n |
| Et | CH(Pr-i)S(O)₂Pr-n |
| Et | CH(Pr-i)SPr-i |
| Et | CH(Pr-i)S(O)Pr-i |
| Et | CH(Pr-i)S(O)₂Pr-i |
| Et | CH(Pr-i)SPr-c |
| Et | CH(Pr-i)S(O)Pr-c |
| Et | CH(Pr-i)S(O)₂Pr-c |
| Et | CH(Pr-i)SCH₂CF₃ |
| Et | CH(Pr-i)S(O)CH₂CF₃ |
| Et | CH(Pr-i)S(O)₂CH₂CF₃ |
| Et | CH(Pr-c)SMe |
| Et | CH(Pr-c)S(O)Me |
| Et | CH(Pr-c)S(O)₂Me |
| Et | CH(Pr-c)SEt |
| Et | CH(Pr-c)S(O)Et |
| Et | CH(Pr-c)S(O)₂Et |
| Et | CH(Pr-c)S(═NCN)Et |
| Et | CH(Pr-c)S(O)(═NCN)Et |
| Et | CH(Pr-c)SPr-n |
| Et | CH(Pr-c)S(O)Pr-n |
| Et | CH(Pr-c)S(O)₂Pr-n |
| Et | CH(Pr-c)SPr-i |
| Et | CH(Pr-c)S(O)Pr-i |
| Et | CH(Pr-c)S(O)₂Pr-i |
| Et | CH(Pr-c)SPr-c |
| Et | CH(Pr-c)S(O)Pr-c |
| Et | CH(Pr-c)S(O)₂Pr-c |
| Et | CH(Pr-c)SCH₂CF₃ |
| Et | CH(Pr-c)S(O)CH₂CF₃ |
| Et | CH(Pr-c)S(O)₂CH₂CF₃ |
| Et | CH(Bu-n)SMe |
| Et | CH(Bu-n)S(O)Me |
| Et | CH(Bu-n)S(O)₂Me |
| Et | CH(Bu-n)SEt |
| Et | CH(Bu-n)S(O)Et |
| Et | CH(Bu-n)S(O)₂Et |
| Et | CH(Bu-n)S(═NCN)Et |
| Et | CH(Bu-n)S(O)(═NCN)Et |
| Et | CH(Bu-n)SPr-n |
| Et | CH(Bu-n)S(O)Pr-n |
| Et | CH(Bu-n)S(O)₂Pr-n |
| Et | CH(Bu-n)SPr-i |
| Et | CH(Bu-n)S(O)Pr-i |
| Et | CH(Bu-n)S(O)₂Pr-i |
| Et | CH(Bu-n)SPr-c |
| Et | CH(Bu-n)S(O)Pr-c |
| Et | CH(Bu-n)S(O)₂Pr-c |
| Et | CH(Bu-n)SCH₂CF₃ |
| Et | CH(Bu-n)S(O)CH₂CF₃ |
| Et | CH(Bu-n)S(O)₂CH₂CF₃ |
| Et | CH(F)SMe |
| Et | CH(F)S(O)Me |
| Et | CH(F)S(O)₂Me |
| Et | CH(F)SEt |
| Et | CH(F)S(O)Et |
| Et | CH(F)S(O)₂Et |
| Et | CH(F)S(═NCN)Et |
| Et | CH(F)S(O)(═NCN)Et |
| Et | CH(F)SCH₂CF₃ |
| Et | CH(F)S(O)CH₂CF₃ |
| Et | CH(F)S(O)₂CH₂CF₃ |
| Et | C(F)₂SMe |
| Et | C(F)₂S(O)Me |
| Et | C(F)₂S(O)₂Me |
| Et | C(F)₂SEt |
| Et | C(F)₂S(O)Et |
| Et | C(F)₂S(O)₂Et |
| Et | C(F)₂S(═NCN)Et |
| Et | C(F)₂S(O)(═NCN)Et |
| Et | C(F)₂SCH₂CF₃ |
| Et | C(F)₂S(O)CH₂CF₃ |
| Et | C(F)₂S(O)₂CH₂CF₃ |
| Et | CH(Cl)SMe |
| Et | CH(Cl)S(O)Me |
| Et | CH(Cl)S(O)₂Me |
| Et | CH(Cl)SEt |
| Et | CH(Cl)S(O)Et |
| Et | CH(Cl)S(O)₂Et |
| Et | CH(Cl)S(═NCN)Et |
| Et | CH(Cl)S(O)(═NCN)Et |
| Et | CH(Cl)SCH₂CF₃ |
| Et | CH(Cl)S(O)CH₂CF₃ |
| Et | CH(Cl)S(O)₂CH₂CF₃ |
| Et | C(Cl)₂SMe |
| Et | C(Cl)₂S(O)Me |
| Et | C(Cl)₂S(O)₂Me |
| Et | C(Cl)₂SEt |
| Et | C(Cl)₂S(O)Et |
| Et | C(Cl)₂S(O)₂Et |
| Et | C(Cl)₂S(═NCN)Et |
| Et | C(Cl)₂S(O)(═NCN)Et |
| Et | C(Cl)₂SCH₂CF₃ |
| Et | C(Cl)₂S(O)CH₂CF₃ |
| Et | C(Cl)₂S(O)₂CH₂CF₃ |
| Et | CH(Br)SMe |
| Et | CH(Br)S(O)Me |
| Et | CH(Br)S(O)₂Me |
| Et | CH(Br)SEt |
| Et | CH(Br)S(O)Et |
| Et | CH(Br)S(O)₂Et |
| Et | CH(Br)S(═NCN)Et |
| Et | CH(Br)S(O)(═NCN)Et |
| Et | CH(Br)SCH₂CF₃ |
| Et | CH(Br)S(O)CH₂CF₃ |
| Et | CH(Br)S(O)₂CH₂CF₃ |
| Et | CH(I)SMe |
| Et | CH(I)S(O)Me |
| Et | CH(I)S(O)₂Me |
| Et | CH(I)SEt |
| Et | CH(I)S(O)Et |
| Et | CH(I)S(O)₂Et |
| Et | CH(I)S(═NCN)Et |
| Et | CH(I)S(O)(═NCN)Et |
| Et | CH(I)SCH₂CF₃ |
| Et | CH(I)S(O)CH₂CF₃ |
| Et | CH(I)S(O)₂CH₂CF₃ |
| Et | CH{OC(O)Me}SMe |
| Et | CH{OC(O)Me}S(O)Me |
| Et | CH{OC(O)Me}S(O)₂Me |
| Et | CH{OC(O)OMe}SEt |
| Et | CH{OC(O)OMe}S(O)Et |
| Et | CH{OC(O)OMe}S(O)₂Et |
| Et | CH{OC(O)OMe}S(═NCN)Et |
| Et | CH{OC(O)OMe}S(O)(═NCN)Et |
| Et | CH{OC(O)OEt}SMe |
| Et | CH{OC(O)OEt}S(O)Me |
| Et | CH{OC(O)OEt}S(O)₂Me |
| Et | CH{OC(O)OEt}SEt |
| Et | CH{OC(O)OEt}S(O)Et |
| Et | CH{OC(O)OEt}S(O)₂Et |
| Et | CH{OC(O)OEt}S(═NCN)Et |
| Et | CH{OC(O)OEt}S(O)(═NCN)Et |
| Et | CH(OMe)SMe |
| Et | CH(OMe)S(O)Me |
| Et | CH(OMe)S(O)₂Me |
| Et | CH(OMe)SEt |
| Et | CH(OMe)S(O)Et |
| Et | CH(OMe)S(O)₂Et |
| Et | CH(OMe)S(═NCN)Et |
| Et | CH(OMe)S(O)(═NCN)Et |
| Et | CH(OEt)SMe |
| Et | CH(OEt)S(O)Me |
| Et | CH(OEt)S(O)₂Me |
| Et | CH(OEt)SEt |
| Et | CH(OEt)S(O)Et |
| Et | CH(OEt)S(O)₂Et |
| Et | CH(OEt)S(═NCN)Et |
| Et | CH(OEt)S(O)(═NCN)Et |
| Et | CH(SMe)₂ |
| Et | CH(SMe)S(O)Me |
| Et | CH(SMe)S(O)₂Me |
| Et | CH(SMe)SEt |
| Et | CH(SMe)S(O)Et |
| Et | CH(SMe)S(O)₂Et |
| Et | CH(SMe)S(═NCN)Et |
| Et | CH(SMe)S(O)(═NCN)Et |
| Et | CH(SEt)SMe |
| Et | CH(SEt)S(O)Me |
| Et | CH(SEt)S(O)₂Me |
| Et | CH(SEt)SEt |
| Et | CH(SEt)S(O)Et |
| Et | CH(SEt)S(O)₂Et |
| Et | CH(SEt)S(═NCN)Et |
| Et | CH(SEt)S(O)(═NCN)Et |
| Et | CH(CN)SMe |
| Et | CH(CN)S(O)Me |
| Et | CH(CN)S(O)₂Me |
| Et | CH(CN)SEt |
| Et | CH(CN)S(O)Et |
| Et | CH(CN)S(O)₂Et |
| Et | CH(CN)S(═NCN)Et |
| Et | CH(CN)S(O)(═NCN)Et |
| Et | CH{C(O)OMe}SMe |
| Et | CH{C(O)OMe}S(O)Me |
| Et | CH{C(O)OMe}S(O)₂Me |
| Et | CH{C(O)OMe}SEt |
| Et | CH{C(O)OMe}S(O)Et |
| Et | CH{C(O)OMe}S(O)₂Et |
| Et | CH{C(O)OMe}S(═NCN)Et |
| Et | CH{C(O)OMe}S(O)(═NCN)Et |
| Et | CH{C(O)OEt}SMe |
| Et | CH{C(O)OEt}S(O)Me |
| Et | CH{C(O)OEt}S(O)₂Me |
| Et | CH{C(O)OEt}SEt |
| Et | CH{C(O)OEt}S(O)Et |
| Et | CH{C(O)OEt}S(O)Et |
| Et | CH{C(O)OEt}S(═NCN)Et |
| Et | CH{C(O)OEt}S(O)(═NCN)Et |
| Et | C(Me)₂SMe |
| Et | C(Me)₂S(O)Me |
| Et | C(Me)₂S(O)₂Me |
| Et | CH₂CH₂SH |
| Et | CH₂CH₂SMe |
| Et | CH₂CH₂S(O)Me |
| Et | CH₂CH₂S(O)₂Me |
| Et | CH₂CH₂SEt |
| Et | CH₂CH₂S(O)Et |
| Et | CH₂CH₂S(O)₂Et |
| Et | CH₂CH₂SPr-n |
| Et | CH₂CH₂S(O)Pr-n |
| Et | CH₂CH₂S(O)₂Pr-n |
| Et | CH₂CH₂SPr-i |
| Et | CH₂CH₂S(O)Pr-i |
| Et | CH₂CH₂S(O)₂Pr-i |
| Et | CH₂CH₂SCH₂CF₃ |
| Et | CH₂CH₂S(O)CH₂CF₃ |
| Et | CH₂CH₂S(O)₂CH₂CF₃ |
| Et | CH₂CH₂SC(O)Me |
| Et | CH(Me)CH₂SMe |
| Et | CH(Me)CH₂S(O)Me |
| Et | CH(Me)CH₂S(O)₂Me |
| Et | CH(Me)CH₂SEt |
| Et | CH(Me)CH₂S(O)Et |
| Et | CH(Me)CH₂S(O)₂Et |
| Et | CH(Me)CH₂SPr-n |
| Et | CH(Me)CH₂S(O)Pr-n |
| Et | CH(Me)CH₂S(O)₂Pr-n |
| Et | CH(Me)CH₂SPr-i |
| Et | CH(Me)CH₂S(O)Pr-i |
| Et | CH(Me)CH₂S(O)₂Pr-i |
| Et | CH(Me)CH₂SCH₂CF₃ |
| Et | CH(Me)CH₂S(O)CH₂CF₃ |
| Et | CH(Me)CH₂S(O)₂CH₂CF₃ |
| Et | CH(Me)CH₂SC(O)Me |
| Et | CH(Et)CH₂SMe |
| Et | CH(Et)CH₂S(O)Me |
| Et | CH(Et)CH₂S(O)₂Me |
| Et | CH(Et)CH₂SEt |
| Et | CH(Et)CH₂S(O)Et |
| Et | CH(Et)CH₂S(O)₂Et |
| Et | CH(Pr-n)CH₂SMe |
| Et | CH(Pr-n)CH₂S(O)Me |
| Et | CH(Pr-n)CH₂S(O)₂Me |
| Et | CH(Pr-n)CH₂SEt |
| Et | CH(Pr-n)CH₂S(O)Et |
| Et | CH(Pr-n)CH₂S(O)₂Et |
| Et | CH(Pr-i)CH₂SMe |
| Et | CH(Pr-i)CH₂S(O)Me |
| Et | CH(Pr-i)CH₂S(O)₂Me |
| Et | CH(Pr-i)CH₂SEt |
| Et | CH(Pr-i)CH₂S(O)Et |
| Et | CH(Pr-i)CH₂S(O)₂Et |
| Et | CH(Pr-c)CH₂SMe |
| Et | CH(Pr-c)CH₂S(O)Me |
| Et | CH(Pr-c)CH₂S(O)₂Me |
| Et | CH(Pr-c)CH₂SEt |
| Et | CH(Pr-c)CH₂S(O)Et |
| Et | CH(Pr-c)CH₂S(O)₂Et |
| Et | CH(Bu-n)CH₂SMe |
| Et | CH(Bu-n)CH₂S(O)Me |
| Et | CH(Bu-n)CH₂S(O)₂Me |
| Et | CH(Bu-n)CH₂SEt |
| Et | CH(Bu-n)CH₂S(O)Et |
| Et | CH(Bu-n)CH₂S(O)₂Et |
| Et | CH(Bu-i)CH₂SMe |
| Et | CH(Bu-i)CH₂S(O)Me |
| Et | CH(Bu-i)CH₂S(O)₂Me |
| Et | CH(Bu-i)CH₂SEt |
| Et | CH(Bu-i)CH₂S(O)Et |
| Et | CH(Bu-i)CH₂S(O)₂Et |
| Et | CH(Bu-s)CH₂SMe |
| Et | CH(Bu-s)CH₂S(O)Me |
| Et | CH(Bu-s)CH₂S(O)₂Me |
| Et | CH(Bu-s)CH₂SEt |
| Et | CH(Bu-s)CH₂S(O)Et |
| Et | CH(Bu-s)CH₂S(O)₂Et |
| Et | CH(Bu-t)CH₂SMe |
| Et | CH(Bu-t)CH₂S(O)Me |
| Et | CH(Bu-t)CH₂S(O)₂Me |
| Et | CH(Bu-t)CH₂SEt |
| Et | CH(Bu-t)CH₂S(O)Et |
| Et | CH(Bu-t)CH₂S(O)₂Et |
| Et | CH(OMe)CH₂SMe |
| Et | CH(OMe)CH₂S(O)Me |
| Et | CH(OMe)CH₂S(O)₂Me |
| Et | CH(OMe)CH₂SEt |
| Et | CH(OMe)CH₂S(O)Et |
| Et | CH(OMe)CH₂S(O)₂Et |
| Et | CH(SMe)CH₂SMe |
| Et | CH(SMe)CH₂S(O)Me |
| Et | CH(SMe)CH₂S(O)₂Me |
| Et | CH(SMe)CH₂SEt |
| Et | CH(SMe)CH₂S(O)Et |
| Et | CH(SMe)CH₂S(O)₂Et |
| Et | CH(CH₂OMe)CH₂SMe |
| Et | CH(CH₂OMe)CH₂S(O)Me |
| Et | CH(CH₂OMe)CH₂S(O)₂Me |
| Et | CH(CH₂OMe)CH₂SEt |
| Et | CH(CH₂OMe)CH₂S(O)Et |
| Et | CH(CH₂OMe)CH₂S(O)₂Et |
| Et | CH(CH₂SMe)CH₂SMe |
| Et | CH(CH₂SMe)CH₂S(O)Me |
| Et | CH(CH₂SMe)CH₂S(O)₂Me |
| Et | CH(CH₂SMe)CH₂SEt |
| Et | CH(CH₂SMe)CH₂S(O)Et |
| Et | CH(CH₂SMe)CH₂S(O)₂Et |
| Et | CH(F)CH₂SMe |
| Et | CH(F)CH₂S(O)Me |
| Et | CH(F)CH₂S(O)₂Me |
| Et | CH(F)CH₂SEt |
| Et | CH(F)CH₂S(O)Et |
| Et | CH(F)CH₂S(O)₂Et |
| Et | C(F)₂CH₂SMe |
| Et | C(F)₂CH₂S(O)Me |
| Et | C(F)₂CH₂S(O)₂Me |
| Et | C(F)₂CH₂SEt |
| Et | C(F)₂CH₂S(O)Et |
| Et | C(F)₂CH₂S(O)₂Et |
| Et | CH(Cl)CH₂SMe |
| Et | CH(Cl)CH₂S(O)Me |
| Et | CH(Cl)CH₂S(O)₂Me |
| Et | CH(Cl)CH₂SEt |
| Et | CH(Cl)CH₂S(O)Et |
| Et | CH(Cl)CH₂S(O)₂Et |
| Et | C(Cl)₂CH₂SMe |
| Et | C(Cl)₂CH₂S(O)Me |
| Et | C(Cl)₂CH₂S(O)₂Me |
| Et | C(Cl)₂CH₂SEt |
| Et | C(Cl)₂CH₂S(O)Et |
| Et | C(Cl)₂CH₂S(O)₂Et |
| Et | CH(Br)CH₂SMe |
| Et | CH(Br)CH₂S(O)Me |
| Et | CH(Br)CH₂S(O)₂Me |
| Et | CH(Br)CH₂SEt |
| Et | CH(Br)CH₂S(O)Et |
| Et | CH(Br)CH₂S(O)₂Et |
| Et | CH(I)CH₂SMe |
| Et | CH(I)CH₂S(O)Me |
| Et | CH(I)CH₂S(O)₂Me |
| Et | CH(I)CH₂SEt |
| Et | CH(I)CH₂S(O)Et |
| Et | CH(I)CH₂S(O)₂Et |
| Et | CH(CN)CH₂SMe |
| Et | CH(CN)CH₂S(O)Me |
| Et | CH(CN)CH₂S(O)₂Me |
| Et | CH(CN)CH₂SEt |
| Et | CH(CN)CH₂S(O)Et |
| Et | CH(CN)CH₂S(O)₂Et |
| Et | CH{C(O)OMe}CH₂SMe |
| Et | CH{C(O)OMe}CH₂S(O)Me |
| Et | CH{C(O)OMe}CH₂S(O)₂Me |
| Et | CH{C(O)OMe}CH₂SEt |
| Et | CH{C(O)OMe}CH₂S(O)Et |
| Et | CH{C(O)OMe}CH₂S(O)₂Et |
| Et | CH{C(O)OEt}CH₂SMe |
| Et | CH{C(O)OEt}CH₂S(O)Me |
| Et | CH{C(O)OEt}CH₂S(O)₂Me |
| Et | CH{C(O)OEt}CH₂SEt |
| Et | CH{C(O)OEt}CH₂S(O)Et |
| Et | CH{C(O)OEt}CH₂S(O)₂Et |
| Et | CH(Ph)CH₂SMe |
| Et | CH(Ph)CH₂S(O)Me |
| Et | CH(Ph)CH₂S(O)₂Me |
| Et | CH(Ph)CH₂SEt |
| Et | CH(Ph)CH₂S(O)Et |
| Et | CH(Ph)CH₂S(O)₂Et |
| Et | C(Me)₂CH₂SMe |
| Et | C(Me)₂CH₂S(O)Me |
| Et | C(Me)₂CH₂S(O)₂Me |
| Et | C(Me)₂CH₂SEt |
| Et | C(Me)₂CH₂S(O)Et |
| Et | C(Me)₂CH₂S(O)₂Et |
| Et | C(Et)₂CH₂SMe |
| Et | C(Et)₂CH₂S(O)Me |
| Et | C(Et)₂CH₂S(O)₂Me |
| Et | C(Et)₂CH₂SEt |
| Et | C(Et)₂CH₂S(O)Et |
| Et | C(Et)₂CH₂S(O)₂Et |
| Et | C(═CH₂)CH₂SMe |
| Et | C(═CH₂)CH₂S(O)Me |
| Et | C(═CH₂)CH₂S(O)₂Me |
| Et | C(═CH₂)CH₂SEt |
| Et | C(═CH₂)CH₂S(O)Et |
| Et | C(═CH₂)CH₂S(O)₂Et |
| Et | CH₂CH₂CH₂SMe |
| Et | CH₂CH₂CH₂S(O)Me |
| Et | CH₂CH₂CH₂S(O)₂Me |
| Et | CH(Me)CH₂CH₂SMe |
| Et | CH(Me)CH₂CH₂S(O)Me |
| Et | CH(Me)CH₂CH₂S(O)₂Me |
| Et | CH₂CH(Me)CH₂SMe |
| Et | CH₂CH(Me)CH₂S(O)Me |
| Et | CH₂CH(Me)CH₂S(O)₂Me |
| Et | CH₂CH₂CH(Me)SMe |
| Et | CH₂CH₂CH(Me)S(O)Me |
| Et | CH₂CH₂CH(Me)S(O)₂Me |
| Et | CH₂CH₂CH₂SEt |
| Et | CH₂CH₂CH₂S(O)Et |
| Et | CH₂CH₂CH₂S(O)₂Et |
| Et | CH(Me)CH₂CH₂SEt |
| Et | CH(Me)CH₂CH₂S(O)Et |
| Et | CH(Me)CH₂CH₂S(O)₂Et |
| Et | CH₂CH(Me)CH₂SEt |
| Et | CH₂CH(Me)CH₂S(O)Et |
| Et | CH₂CH(Me)CH₂S(O)₂Et |
| Et | CH₂CH₂CH(Me)SEt |
| Et | CH₂CH₂CH(Me)S(O)Et |
| Et | CH₂CH₂CH(Me)S(O)₂Et |
| Et | CH₂CH₂CH₂SPr-n |
| Et | CH₂CH₂CH₂S(O)Pr-n |
| Et | CH₂CH₂CH₂S(O)₂Pr-n |
| Et | CH(Me)CH₂CH₂SPr-n |
| Et | CH(Me)CH₂CH₂S(O)Pr-n |
| Et | CH(Me)CH₂CH₂S(O)₂Pr-n |
| Et | CH₂CH(Me)CH₂SPr-n |
| Et | CH₂CH(Me)CH₂S(O)Pr-n |
| Et | CH₂CH(Me)CH₂S(O)₂Pr-n |
| Et | CH₂CH₂CH₂CH₂SMe |
| Et | CH₂CH₂CH₂CH₂S(O)Me |
| Et | CH₂CH₂CH₂CH₂S(O)₂Me |
| Et | CH₂CH₂CH₂CH₂SEt |
| Et | CH₂CH₂CH₂CH₂S(O)Et |
| Et | CH₂CH₂CH₂CH₂S(O)₂Et |
| Et | CH₂CH₂CH₂CH₂SPr-n |
| Et | CH₂CH₂CH₂CH₂S(O)Pr-n |
| Et | CH₂CH₂CH₂CH₂S(O)₂Pr-n |
| Et | CH₂CH(Me)SMe |
| Et | CH₂CH(Me)S(O)Me |
| Et | CH₂CH(Me)S(O)₂Me |
| Et | CH₂CH(Me)SEt |
| Et | CH₂CH(Me)S(O)Et |
| Et | CH₂CH(Me)S(O)₂Et |
| Et | CH₂CH(Et)SMe |
| Et | CH₂CH(Et)S(O)Me |
| Et | CH₂CH(Et)S(O)₂Me |
| Et | CH₂CH(Et)SEt |
| Et | CH₂CH(Et)S(O)Et |
| Et | CH₂CH(Et)S(O)₂Et |
| Et | CH₂CH(Pr-n)SMe |
| Et | CH₂CH(Pr-n)S(O)Me |
| Et | CH₂CH(Pr-n)S(O)₂Me |
| Et | CH₂CH(Pr-n)SEt |
| Et | CH₂CH(Pr-n)S(O)Et |
| Et | CH₂CH(Pr-n)S(O)₂Et |
| Et | CH₂CH(Pr-i)SMe |
| Et | CH₂CH(Pr-i)S(O)Me |
| Et | CH₂CH(Pr-i)S(O)₂Me |
| Et | CH₂CH(Pr-i)SEt |
| Et | CH₂CH(Pr-i)S(O)Et |
| Et | CH₂CH(Pr-i)S(O)₂Et |
| Et | CH₂CH(Pr-c)SMe |
| Et | CH₂CH(Pr-c)S(O)Me |
| Et | CH₂CH(Pr-c)S(O)₂Me |
| Et | CH₂CH(Pr-c)SEt |
| Et | CH₂CH(Pr-c)S(O)Et |
| Et | CH₂CH(Pr-c)S(O)₂Et |
| Et | CH₂CH(Bu-n)SMe |
| Et | CH₂CH(Bu-n)S(O)Me |
| Et | CH₂CH(Bu-n)S(O)₂Me |
| Et | CH₂CH(Bu-n)SEt |
| Et | CH₂CH(Bu-n)S(O)Et |
| Et | CH₂CH(Bu-n)S(O)₂Et |
| Et | CH₂CH(Bu-i)SMe |
| Et | CH₂CH(Bu-i)S(O)Me |
| Et | CH₂CH(Bu-i)S(O)₂Me |
| Et | CH₂CH(Bu-i)SEt |
| Et | CH₂CH(Bu-i)S(O)Et |
| Et | CH₂CH(Bu-i)S(O)₂Et |
| Et | CH₂CH(Bu-s)SMe |
| Et | CH₂CH(Bu-s)S(O)Me |
| Et | CH₂CH(Bu-s)S(O)₂Me |
| Et | CH₂CH(Bu-s)SEt |
| Et | CH₂CH(Bu-s)S(O)Et |
| Et | CH₂CH(Bu-s)S(O)₂Et |
| Et | CH₂CH(Bu-t)SMe |
| Et | CH₂CH(Bu-t)S(O)Me |
| Et | CH₂CH(Bu-t)S(O)₂Me |
| Et | CH₂CH(Bu-t)SEt |
| Et | CH₂CH(Bu-t)S(O)Et |
| Et | CH₂CH(Bu-t)S(O)₂Et |
| Et | CH₂C(Et)₂SMe |
| Et | CH₂C(Et)₂S(O)Me |
| Et | CH₂C(Et)₂S(O)₂Me |
| Et | CH₂C(Et)₂SEt |
| Et | CH₂C(Et)₂S(O)Et |
| Et | CH₂C(Et)₂S(O)₂Et |
| Et | CH(Et)CH(Me)SMe |
| Et | CH(Et)CH(Me)S(O)Me |
| Et | CH(Et)CH(Me)S(O)₂Me |
| Et | CH(Et)CH(Me)SEt |
| Et | CH(Et)CH(Me)S(O)Et |
| Et | CH(Et)CH(Me)S(O)₂Et |
| Et | CH(Et)CH(Et)SMe |
| Et | CH(Et)CH(Et)S(O)Me |
| Et | CH(Et)CH(Et)S(O)₂Me |
| Et | CH(Et)CH(Et)SEt |
| Et | CH(Et)CH(Et)S(O)Et |
| Et | CH(Et)CH(Et)S(O)₂Et |
| Et | CH═CHSMe |
| Et | CH₂CH(SMe)₂ |
| Et | CH₂CH₂SC(O)Me |
| Et | C(CH₂)CH₂C(O)OH |
| Et | C(═NOMe)Me |
| Et | C(═NOMe)Et |
| Et | C(═NOMe)Pr-n |
| Et | C(═NOEt)Me |
| Et | C(═NOPr-n)Me |
| Et | C(═NOPr-i)Me |
| Et | C(═NOBu-n)Me |
| Et | C(═NOCH₂Ph)Me |
| Et | C(═NOMe)C(═NOMe)Me |
| Et | C(═NOMe)CH₂OCH₂CF₃ |
| Et | C(═NOMe)CH₂SMe |
| Et | C(═NOMe)CH₂S(O)Me |
| Et | C(═NOMe)CH₂S(O)₂Me |
| Et | C(═NOMe)CH₂SEt |
| Et | C(═NOMe)CH₂S(O)Et |
| Et | C(═NOMe)CH₂S(O)₂Et |
| Et | C(═NOMe)CH₂SCF₃ |
| Et | C(═NOMe)CH₂S(O)CF₃ |
| Et | C(═NOMe)CH₂S(O)₂CF₃ |
| Et | C(═NOMe)CH₂SCH₂CF₃ |
| Et | C(═NOMe)CH₂S(O)CH₂CF₃ |
| Et | C(═NOMe)CH₂S(O)₂CH₂CF₃ |
| Et | C(═NOEt)CH₂SMe |
| Et | C(═NOEt)CH₂S(O)Me |
| Et | C(═NOEt)CH₂S(O)₂Me |
| Et | C(═NOPr-n)CH₂SMe |
| Et | C(═NOPr-n)CH₂S(O)Me |
| Et | C(═NOPr-n)CH₂S(O)₂Me |
| Et | C(═NOPr-i)CH₂SMe |
| Et | C(═NOPr-i)CH₂S(O)Me |
| Et | C(═NOPr-i)CH₂S(O)₂Me |
| Et | C(═NOBu-n)CH₂SMe |
| Et | C(═NOBu-n)CH₂S(O)Me |
| Et | C(═NOBu-n)CH₂S(O)₂Me |
| Et | C(═NOCH₂Ph)CH₂SMe |
| Et | C(═NOCH₂Ph)CH₂S(O)Me |
| Et | C(═NOCH₂Ph)CH₂S(O)₂Me |
| Et | C(═NOCH₂CH═CH₂)CH₂SMe |
| Et | C(═NOCH₂OMe)CH₂SMe |
| Et | C(═NOCH₂CH₂OMe)CH₂SMe |
| Et | C{═NOCH₂CH₂Si(Me)₃}CH₂SMe |
| Et | CH₂CH(═NOMe) |
| Et | CH₂C(═NOMe)Me |
| Et | CH₂C(═NOMe)Et |
| Et | CH₂C(═NOMe)Pr-n |
| Et | CH₂C(═NOMe)Pr-i |
| Et | CH₂C(═NOMe)Bu-n |
| Et | CH₂C(═NOMe)Bu-i |
| Et | CH₂C(═NOMe)Bu-s |
| Et | CH₂C(═NOMe)Bu-t |
| Et | CH₂C(═NOMe)CF₃ |
| Et | CH₂C(═NOCH₃)CH₂SCH₃ |
| Et | CH(SMe)C(═NOMe)Me |
| Et | CH{S(O)Me}C(═NOMe)Me |
| Et | CH{S(O)₂Me}C(═NOMe)Me |
| Et | CH(SEt)C(═NOMe)Me |
| Et | CH{S(O)Et}C(═NOMe)Me |
| Et | CH{S(O)₂Et}C(═NOMe)Me |
| Et | CH₂NH₂ |
| Et | CH₂NMe₂ |
| Et | CH₂NHC(O)Me |
| Et | CH₂NHC(O)CF₃ |
| Et | CH₂NHC(O)CH₂OMe |
| Et | CH₂NHC(O)CH₂SMe |
| Et | CH₂NHC(O)OMe |
| Et | CH₂NHC(O)OBu-t |
| Et | CH₂NHSO₂Me |
| Et | CH₂NHSO₂NMe₂ |
| Et | CH₂NHSO₂CF₃ |
| Et | CH₂N(Me)C(O)Me |
| Et | CH₂N(Me)C(O)OMe |
| Et | CH₂N(Me)C(O)OBu-t |
| Et | CH₂N(Me)SO₂Me |
| Et | CH(Me)NHC(O)OBu-t |
| Et | CH(CH₂SMe)NH₂ |
| Et | CH(CH₂SMe)NHC(O)Me |
| Et | CH(CH₂SMe)NHC(O)OMe |
| Et | CH(CH₂SMe)NHC(O)OBu-t |
| Et | CH(CH₂SMe)NHSO₂Me |
| Et | CH₂NHC(O)NHMe |
| Et | CH₂NHC(O)NHEt |
| Et | CH₂NHC(O)NHPr-n |
| Et | CH₂NHC(O)NHPr-i |
| Et | CH₂NHC(O)NHPr-c |
| Et | CH₂NHC(S)NHMe |
| Et | CH₂NHC(S)NHEt |
| Et | CH₂NHC(S)NHPr-n |
| Et | CH₂NHC(S)NHPr-i |
| Et | CH₂NHC(S)NHPr-c |
| Et | CH₂CH₂NHC(O)OBu-t |
| Et | CH₂P(O)(OMe)₂ |
| Et | CH₂P(O)(OEt)₂ |
| Et | SMe |
| Et | OBu-t |
| Et | O{D1-108(4-NO₂)} |
| Et | NH₂ |
| Et | NHCH₂SMe |
| Et | NHCH₂CH₂SMe |
| Et | N(Me)CH₂SMe |
| Et | N(Me)CH₂CH₂SMe |
| Et | NHCH₂CH₂CH₂SMe |
| Et | NHMe |
| Et | NHEt |
| Et | NPr-n |
| Et | NHPr-i |
| Et | NHCH₂C(O)OMe |
| Et | NHCH₂CF₃ |
| Et | N(Me)CH₂CF₃ |
| Et | NHC(O)OEt |
| Et | NHC(O)CCl₃ |
| Et | NHC(O)CH₂SMe |
| Et | N(CH₂CH₃)C(O)CH₂SCH₃ |
| Et | NHS(O)₂(D1-108a) |
| Et | NHPh |
| Et | NH(D1-1a) |
| Et | NH(D1-2a) |
| Et | NH(D1-2b) |
| Et | NH{D1-4a(X^{1a}═Me)} |
| Et | NH{D1-4b(X^{1a}═Me)} |
| Et | NH(D1-5a) |
| Et | NH(D1-5b) |
| Et | NH(D1-5c) |
| Et | NH(D1-6a) |
| Et | NH(D1-6b) |
| Et | NH(D1-6c) |
| Et | NH(D1-7a) |
| Et | NH{D1-8a(X^{1a}═Me)} |
| Et | NH{D1-8b(X^{1a}═Me)} |
| Et | NH{D1-8c(X^{1a}═Me)} |
| Et | NH(D1-9a) |
| Et | NH(D1-9b) |
| Et | NH(D1-9c) |
| Et | NH(D1-10a) |
| Et | NH(D1-10b) |
| Et | NH(D1-10c) |
| Et | NH(D1-11a) |
| Et | NH{D1-12a(X^{1a}═Me)} |
| Et | NH{D1-12b(X^{1a}═Me)} |
| Et | NH{D1-12c(X^{1a}═Me)} |
| Et | NH(D1-13a) |
| Et | NH(D1-13b) |
| Et | NH(D1-14a) |
| Et | NH(D1-14b) |
| Et | NH(D1-15a) |
| Et | NH(D1-15b) |
| Et | NH(D1-16a) |
| Et | NH(D1-17a) |
| Et | NH(D1-18a) |
| Et | NH(D1-19a) |
| Et | NH(D1-20a) |
| Et | NH{D1-21a(X^{1a}═Me)} |
| Et | NH{D1-21b(X^{1a}═Me)} |
| Et | NH(D1-22a) |
| Et | NH{D1-23a(X^{1a}═Me)} |
| Et | NH{D1-23b(X^{1a}═Me)} |
| Et | NH(D1-24a) |
| Et | NH{D1-25a(X^{1a}═Me)} |
| Et | NH(D1-26a) |
| Et | NH{D1-27a(X^{1a}═Me)} |
| Et | NH(D1-28a) |
| Et | NH(D1-29a) |
| Et | NH{D1-30a(X^{1a}═Me)} |
| Et | NH{D1-31a(X^{1a}═Me)} |
| Et | NH(D1-32a) |
| Et | NH(D1-33a) |
| Et | NH(D1-34a) |
| Et | NH(D1-35a) |
| Et | NH(D1-35b) |
| Et | NH(D1-36a) |
| Et | NH(D1-37a) |
| Et | NH(D1-38a) |
| Et | NH(D1-39a) |
| Et | NH(D1-40a) |
| Et | NH(D1-40b) |
| Et | NH(D1-40c) |
| Et | NH(D1-41a) |
| Et | NH(D1-42a) |
| Et | NH(D1-43a) |
| Et | NH(D1-45a) |
| Et | NH(D1-49a) |
| Et | NH(D1-51a) |
| Et | NH(D1-59a) |
| Et | NH(D1-61a) |
| Et | NH(D1-79a) |
| Et | NH(D1-80a) |
| Et | NH{D1-108b(2-Cl)} |
| Et | NH{D1-108b(3-Cl)} |
| Et | NH{D1-108b(4-Cl)} |
| Et | NH{D1-108b(2-Me)} |
| Et | NH{D1-108b(3-Me)} |
| Et | NH{D1-108b(4-Me)} |
| Et | N(Me)Ph |
| Et | N(Me)(D1-1a) |
| Et | N(Me)(D1-2a) |
| Et | N(Me)(D1-2b) |
| Et | N(Me){D1-4a(X^{1a}═Me)} |
| Et | N(Me){D1-4b(X^{1a}═Me)} |
| Et | N(Me)(D1-5a) |
| Et | N(Me)(D1-5b) |
| Et | N(Me)(D1-5c) |
| Et | N(Me)(D1-6a) |
| Et | N(Me)(D1-6b) |
| Et | N(Me)(D1-6c) |
| Et | N(Me)(D1-7a) |
| Et | N(Me){D1-8a(X^{1a}═Me)} |
| Et | N(Me){D1-8b(X^{1a}═Me)} |
| Et | N(Me){D1-8c(X^{1a}═Me)} |
| Et | N(Me)(D1-9a) |
| Et | N(Me)(D1-9b) |
| Et | N(Me)(D1-9c) |
| Et | N(Me)(D1-10a) |
| Et | N(Me)(D1-10b) |
| Et | N(Me)(D1-10c) |
| Et | N(Me)(D1-11a) |
| Et | N(Me){D1-12a(X^{1a}═Me)} |
| Et | N(Me){D1-12b(X^{1a}═Me)} |
| Et | N(Me){D1-12c(X^{1a}═Me)} |
| Et | N(Me)(D1-13a) |
| Et | N(Me)(D1-13b) |
| Et | N(Me)(D1-14a) |
| Et | N(Me)(D1-14b) |
| Et | N(Me)(D1-15a) |
| Et | N(Me)(D1-15b) |
| Et | N(Me)(D1-16a) |
| Et | N(Me)(D1-17a) |
| Et | N(Me)(D1-18a) |
| Et | N(Me)(D1-19a) |
| Et | N(Me)(D1-20a) |
| Et | N(Me){D1-21a(X^{1a}═Me)} |
| Et | N(Me){D1-21b(X^{1a}═Me)} |
| Et | N(Me)(D1-22a) |
| Et | N(Me){D1-23a(X^{1a}═Me)} |
| Et | N(Me){D1-23b(X^{1a}═Me)} |
| Et | N(Me)(D1-24a) |
| Et | N(Me){D1-25a(X^{1a}═Me)} |
| Et | N(Me)(D1-26a) |
| Et | N(Me){D1-27a(X^{1a}═Me)} |
| Et | N(Me)(D1-28a) |
| Et | N(Me)(D1-29a) |
| Et | N(Me){D1-30a(X^{1a}═Me)} |
| Et | N(Me){D1-31a(X^{1a}═Me)} |
| Et | N(Me)(D1-32a) |
| Et | N(Me)(D1-33a) |
| Et | N(Me)(D1-34a) |
| Et | N(Me)(D1-35a) |
| Et | N(Me)(D1-35b) |
| Et | N(Me)(D1-36a) |
| Et | N(Me)(D1-37a) |
| Et | N(Me)(D1-38a) |
| Et | N(Me)(D1-39a) |
| Et | N(Me)(D1-40a) |
| Et | N(Me)(D1-40b) |
| Et | N(Me)(D1-40c) |
| Et | N(Me)(D1-41a) |
| Et | N(Me)(D1-42a) |
| Et | N(Me)(D1-43a) |
| Et | N(Me)(D1-45a) |
| Et | N(Me)(D1-49a) |
| Et | N(Me)(D1-51a) |
| Et | N(Me)(D1-59a) |
| Et | N(Me)(D1-61a) |
| Et | N(Me)(D1-79a) |
| Et | N(Me)(D1-80a) |
| Et | N(Me){D1-108b(2-Cl)} |
| Et | N(Me){D1-108b(3-Cl)} |
| Et | N(Me){D1-108b(4-Cl)} |
| Et | N(Me){D1-108b(2-Me)} |
| Et | N(Me){D1-108b(3-Me)} |
| Et | N(Me){D1-108b(4-Me)} |
| Et | D1-8c(CH₂CF₃) |
| Et | D1-1b(5-Br) |
| Et | D1-2c(5-SMe) |
| Et | D1-2c{5-C(O)Me} |
| Et | D1-5d{5-(Pr-c)} |
| Et | D1-5e(3,5-Me₂) |
| Et | D1-6d(4,5-Cl₂) |
| Et | D1-8f{X^{1a}═Me, X═3-Cl, 5-S(O)₂NH₂} |
| Et | D1-4a(X^{1a}═Me) |
| Et | D1-9b |
| Et | D1-9f(4-CH₃) |
| Et | D1-10e(2-CF₃) |
| Et | D1-10f{2-(CH₂SCH₃)-4-CH₃} |
| Et | D1-10f{2-(CH₂OCH₃)-4-CF₃} |
| Et | D1-12f(X^{1a}═Me, X¹═4-NO₂) |
| Et | D1-13d(X^{1a}═Me) |
| Et | D1-19a |
| Et | D1-32b(2-Br) |
| Et | D1-33a |
| Et | D1-33b(2-OMe) |
| Et | D1-33b(6-CN) |
| Et | D1-34a |
| Et | D1-35b |
| Et | D1-38b(4-SMe) |
| Et | D1-39b(5-Me) |
| Et | D1-45d(5,5-Me₂) |
| Et | D1-81a |
| Et | D1-81b |
| Et | D1-82a |
| Et | D1-82b |
| Et | D1-82c |
| Et | D1-84d{X^{1b}═C(Cl)₃} |
| Et | D1-85d{X^{1b}═C(Cl)₃} |
| Et | D1-87a |
| Et | D1-88a{X^{1a}═C(O)Me} |
| Et | D1-88a{X^{1a}═C(O)CH₂SMe} |
| Et | D1-88a{X^{1a}═C(O)OBu-t} |
| Et | D1-88a{X^{1a}═C(O)CF₃} |
| Et | D1-92c |
| Et | D1-94b{X^{1a}═C(O)OBu-t} |
| Et | D1-94c{X^{1a}═C(O)OBu-t} |
| Et | D1-103b |
| Et | D1-103c |
| Et | D1-103d |
| Et | D1-103e |
| Et | D1-103f |
| Et | D1-103g |
| Et | D1-103h |
| Et | D1-103i |
| Et | D1-108b(2-Cl) |
| Et | D1-108b(3-Cl) |
| Et | D1-108b(4-Cl) |
| Et | D1-108b(2-SMe) |
| Et | D1-108b(3-SMe) |
| Et | D1-108b(4-SMe) |
| Et | D1-108b(3-SCF₃) |
| Et | D1-108b(3,4-OCF₂O—) |
| Et | CH₂(D1-2a) |
| Et | CH₂{D1-5f(3-Me)} |
| Et | CH₂{D1-8f(X^{1a}═Me, X¹═3,5-Me₂)} |
| Et | CH₂{D1-8f(X^{1a}═Me, X¹═3,5-Cl₂)} |
| Et | CH₂{D1-9e(2-Ph, 5-Me)} |
| Et | CH₂(D1-28a) |
| Et | CH₂(D1-32a) |
| Et | CH₂(D1-33a) |
| Et | CH₂{D1-33b(6-Cl)} |
| Et | CH₂(D1-34a) |
| Et | CH₂{D1-84d[X^{1b}═C(Cl)₃]} |
| Et | CH₂{D1-85d[X^{1b}═C(Cl)₃]} |
| Et | CH₂(D1-87a) |
| Et | CH₂(D1-93a) |
| Et | CH₂(D1-98a) |
| Et | CH₂{D1-108b(2,3-OCH₂O—)} |
| Et | CH₂CH₂{D1-7b(3-CF₃, 5-Me) |
| Et | CH₂CH₂{D1-7b(3-CF₃, 5-Pr-c) |
| n-Pr | CH(Me)SMe |
| n-Pr | CH(Me)S(O)Me |
| n-Pr | CH(Me)S(O)₂Me |
| n-Pr | CH(Me)SEt |
| n-Pr | CH(Me)S(O)Et |
| n-Pr | CH(Me)S(O)₂Et |
| n-Pr | CH₂CH₂SMe |
| n-Pr | CH₂CH₂S(O)Me |
| n-Pr | CH₂CH₂S(O)₂Me |
| n-Pr | CH(Me)CH₂SMe |
| n-Pr | CH(Me)CH₂S(O)Me |
| n-Pr | CH(Me)CH₂S(O)₂Me |
| n-Pr | OBu-t |
| i-Pr | CH(Me)SMe |
| i-Pr | CH(Me)S(O)Me |
| i-Pr | CH(Me)S(O)₂Me |
| i-Pr | CH(Me)SEt |
| i-Pr | CH(Me)S(O)Et |
| i-Pr | CH(Me)S(O)₂Et |
| i-Pr | CH₂CH₂SMe |
| i-Pr | CH₂CH₂S(O)Me |
| i-Pr | CH₂CH₂S(O)₂Me |
| i-Pr | CH(Me)CH₂SMe |
| i-Pr | CH(Me)CH₂S(O)Me |
| i-Pr | CH(Me)CH₂S(O)₂Me |
| i-Pr | OBu-t |
| CH₂Pr-c | CH(Me)SMe |
| CH₂Pr-c | CH(Me)S(O)Me |
| CH₂Pr-c | CH(Me)S(O)₂Me |
| CH₂Pr-c | CH(Me)SEt |
| CH₂Pr-c | CH(Me)S(O)Et |
| CH₂Pr-c | CH(Me)S(O)₂Et |
| CH₂Pr-c | CH₂CH₂SMe |
| CH₂Pr-c | CH₂CH₂S(O)Me |
| CH₂Pr-c | CH₂CH₂S(O)₂Me |
| CH₂Pr-c | CH(Me)CH₂SMe |
| CH₂Pr-c | CH(Me)CH₂S(O)Me |
| CH₂Pr-c | CH(Me)CH₂S(O)₂Me |
| CH₂Pr-c | OBu-t |
| CH₂CH═CH₂ | CH(Me)SMe |
| CH₂CH═CH₂ | CH(Me)S(O)Me |
| CH₂CH═CH₂ | CH(Me)S(O)₂Me |
| CH₂CH═CH₂ | CH(Me)SEt |
| CH₂CH═CH₂ | CH(Me)S(O)Et |
| CH₂CH═CH₂ | CH(Me)S(O)₂Et |
| CH₂CH═CH₂ | CH₂CH₂SMe |
| CH₂CH═CH₂ | CH₂CH₂S(O)Me |
| CH₂CH═CH₂ | CH₂CH₂S(O)₂Me |
| CH₂CH═CH₂ | CH(Me)CH₂SMe |
| CH₂CH═CH₂ | CH(Me)CH₂S(O)Me |
| CH₂CH═CH₂ | CH(Me)CH₂S(O)₂Me |
| CH₂C≡CH | CH(Me)SMe |
| CH₂C≡CH | CH(Me)S(O)Me |
| CH₂C≡CH | CH(Me)S(O)₂Me |
| CH₂C≡CH | CH(Me)SEt |
| CH₂C≡CH | CH(Me)S(O)Et |
| CH₂C≡CH | CH(Me)S(O)₂Et |
| CH₂C≡CH | CH₂CH₂SMe |
| CH₂C≡CH | CH₂CH₂S(O)Me |
| CH₂C≡CH | CH₂CH₂S(O)₂Me |
| CH₂C≡CH | CH(Me)CH₂SMe |
| CH₂C≡CH | CH(Me)CH₂S(O)Me |
| CH₂C≡CH | CH(Me)CH₂S(O)₂Me |
| CH₂OMe | CH(Me)SMe |
| CH₂OMe | CH(Me)S(O)Me |
| CH₂OMe | CH(Me)S(O)₂Me |
| CH₂OMe | CH(Me)SEt |
| CH₂OMe | CH(Me)S(O)Et |
| CH₂OMe | CH(Me)S(O)₂Et |
| CH₂OMe | CH₂CH₂SMe |
| CH₂OMe | CH₂CH₂S(O)Me |
| CH₂OMe | CH₂CH₂S(O)₂Me |
| CH₂OMe | CH(Me)CH₂SMe |
| CH₂OMe | CH(Me)CH₂S(O)Me |
| CH₂OMe | CH(Me)CH₂S(O)₂Me |
| CH₂OEt | CH(Me)SEt |
| CH₂OEt | CH(Me)S(O)Et |
| CH₂OEt | CH(Me)S(O)₂Et |
| CH₂CH₂OMe | CH(Me)SEt |
| CH₂CH₂OMe | CH(Me)S(O)Et |
| CH₂CH₂OMe | CH(Me)S(O)₂Et |
| CH₂CN | CH(Me)SMe |
| CH₂CN | CH(Me)S(O)Me |
| CH₂CN | CH(Me)S(O)₂Me |
| CH₂CN | CH(Me)SEt |
| CH₂CN | CH(Me)S(O)Et |
| CH₂CN | CH(Me)S(O)₂Et |
| CH₂CN | CH₂CH₂SMe |
| CH₂CN | CH₂CH₂S(O)Me |
| CH₂CN | CH₂CH₂S(O)₂Me |
| CH₂CN | CH(Me)CH₂SMe |
| CH₂CN | CH(Me)CH₂S(O)Me |
| CH₂CN | CH(Me)CH₂S(O)₂Me |
| CH₂CN | OBu-t |
| CH₂C(O)OMe | CH₂SMe |
| CH₂C(O)OMe | CH₂S(O)Me |
| CH₂C(O)OMe | CH₂S(O)₂Me |
| CH₂C(O)OMe | CH₂SEt |
| CH₂C(O)OMe | CH₂S(O)Et |
| CH₂C(O)OMe | CH₂S(O)₂Et |
| CH₂C(O)OMe | CH₂SPr-n |
| CH₂C(O)OMe | CH₂S(O)Pr-n |
| CH₂C(O)OMe | CH₂S(O)₂Pr-n |
| CH₂C(O)OMe | CH₂SPr-i |
| CH₂C(O)OMe | CH₂S(O)Pr-i |
| CH₂C(O)OMe | CH₂S(O)₂Pr-i |
| CH₂C(O)OMe | CH₂SPr-c |
| CH₂C(O)OMe | CH₂S(O)Pr-c |
| CH₂C(O)OMe | CH₂S(O)₂Pr-c |
| CH₂C(O)OMe | CH₂SCH₂CH═CH₂ |
| CH₂C(O)OMe | CH₂S(O)CH₂CH═CH₂ |
| CH₂C(O)OMe | CH₂S(O)₂CH₂CH═CH₂ |
| CH₂C(O)OMe | CH₂SCH₂C≡CH |
| CH₂C(O)OMe | CH₂S(O)CH₂C≡CH |
| CH₂C(O)OMe | CH₂S(O)₂CH₂C≡CH |
| CH₂C(O)OMe | CH₂SCH₂CF₃ |
| CH₂C(O)OMe | CH₂S(O)CH₂CF₃ |
| CH₂C(O)OMe | CH₂S(O)₂CH₂CF₃ |
| CH₂C(O)OMe | CH₂SCH₂CH(OMe)₂ |
| CH₂C(O)OMe | CH₂SCH₂CH(═NOMe) |
| CH₂C(O)OMe | CH(Me)SMe |
| CH₂C(O)OMe | CH(Me)S(O)Me |
| CH₂C(O)OMe | CH(Me)S(O)₂Me |
| CH₂C(O)OMe | CH(Me)SEt |
| CH₂C(O)OMe | CH(Me)S(O)Et |
| CH₂C(O)OMe | CH(Me)S(O)₂Et |
| CH₂C(O)OMe | CH(Me)S(═NCN)Et |
| CH₂C(O)OMe | CH(Me)S(O)(═NCN)Et |
| CH₂C(O)OMe | CH(Me)SPr-n |
| CH₂C(O)OMe | CH(Me)S(O)Pr-n |
| CH₂C(O)OMe | CH(Me)S(O)₂Pr-n |
| CH₂C(O)OMe | CH(Me)SPr-i |
| CH₂C(O)OMe | CH(Me)S(O)Pr-i |
| CH₂C(O)OMe | CH(Me)S(O)₂Pr-i |
| CH₂C(O)OMe | CH(Me)SBu-t |
| CH₂C(O)OMe | CH(Me)S(O)Bu-t |
| CH₂C(O)OMe | CH(Me)S(O)₂Bu-t |
| CH₂C(O)OMe | CH(Me)SCH₂Pr-c |
| CH₂C(O)OMe | CH(Me)S(O)CH₂Pr-c |
| CH₂C(O)OMe | CH(Me)S(O)₂CH₂Pr-c |
| CH₂C(O)OMe | CH(Me)SCH₂OMe |
| CH₂C(O)OMe | CH(Me)SCH₂SMe |
| CH₂C(O)OMe | CH(Me)S(O)CH₂SMe |
| CH₂C(O)OMe | CH(Me)S(O)₂CH₂SMe |
| CH₂C(O)OMe | CH(Me)SCH₂C═CH₂ |
| CH₂C(O)OMe | CH(Me)S(O)CH₂C═CH₂ |
| CH₂C(O)OMe | CH(Me)S(O)₂CH₂C═CH₂ |
| CH₂C(O)OMe | CH(Me)SCH₂C≡CH |
| CH₂C(O)OMe | CH(Me)S(O)CH₂C≡CH |
| CH₂C(O)OMe | CH(Me)S(O)₂CH₂C≡CH |
| CH₂C(O)OMe | CH(Me)SCH₂C(O)NHMe |
| CH₂C(O)OMe | CH(Me)S(O)CH₂C(O)NHMe |
| CH₂C(O)OMe | CH(Me)S(O)₂CH₂C(O)NHMe |
| CH₂C(O)OMe | CH(Me)SCH₂C(O)OMe |
| CH₂C(O)OMe | CH(Me)S(O)CH₂C(O)OMe |
| CH₂C(O)OMe | CH(Me)S(O)₂CH₂C(O)OMe |
| CH₂C(O)OMe | CH(Me)SCH₂CH₂CH₂Cl |
| CH₂C(O)OMe | CH(Me)S(O)CH₂CH₂CH₂Cl |
| CH₂C(O)OMe | CH(Me)S(O)₂CH₂CH₂CH₂Cl |
| CH₂C(O)OMe | CH(Me)SCH₂CF₃ |
| CH₂C(O)OMe | CH(Me)S(O)CH₂CF₃ |
| CH₂C(O)OMe | CH(Me)S(O)₂CH₂CF₃ |
| CH₂C(O)OMe | CH(Me)SCH₂Ph |
| CH₂C(O)OMe | CH(Me)S(O)CH₂Ph |
| CH₂C(O)OMe | CH(Me)S(O)₂CH₂Ph |
| CH₂C(O)OMe | CH(Me)SCH₂(D1-34a) |
| CH₂C(O)OMe | H(Me)S(O)CH₂(D1-34a) |
| CH₂C(O)OMe | H(Me)S(O)₂CH₂(D1-34a) |
| CH₂C(O)OMe | CH(CH₃)SCH₂Si(CH₃)₃ |
| CH₂C(O)OMe | CH(Me)SCN |
| CH₂C(O)OMe | CH(Me)SCH₂CN |
| CH₂C(O)OMe | CH(Me)SC(O)Me |
| CH₂C(O)OMe | CH(Me)S{D1-12a(X^{1a}═Me)} |
| CH₂C(O)OMe | CH(Me)S(D1-32a) |
| CH₂C(O)OMe | CH(Me)S{D1-32b(3-NO₂)} |
| CH₂C(O)OMe | CH(Me)S{D1-32b(3-CF₃)} |
| CH₂C(O)OMe | CH(Me)S(D1-37a) |
| CH₂C(O)OMe | CH(Me)S(D1-51a) |
| CH₂C(O)OMe | CH(Et)SMe |
| CH₂C(O)OMe | CH(Et)S(O)Me |
| CH₂C(O)OMe | CH(Et)S(O)₂Me |
| CH₂C(O)OMe | CH(Et)SEt |
| CH₂C(O)OMe | CH(Et)S(O)Et |
| CH₂C(O)OMe | CH(Et)S(O)₂Et |
| CH₂C(O)OMe | CH(Et)SCH₂CF₃ |
| CH₂C(O)OMe | CH(Et)S(O)CH₂CF₃ |
| CH₂C(O)OMe | CH(Et)S(O)₂CH₂CF₃ |
| CH₂C(O)OMe | CH(Pr-n)SMe |
| CH₂C(O)OMe | CH(Pr-n)S(O)Me |
| CH₂C(O)OMe | CH(Pr-n)S(O)₂Me |
| CH₂C(O)OMe | CH(Pr-n)SEt |
| CH₂C(O)OMe | CH(Pr-n)S(O)Et |
| CH₂C(O)OMe | CH(Pr-n)S(O)₂Et |
| CH₂C(O)OMe | CH(Pr-n)S(═NCN)Et |
| CH₂C(O)OMe | CH(Pr-n)S(O)(═NCN)Et |
| CH₂C(O)OMe | CH(Pr-n)SPr-n |
| CH₂C(O)OMe | CH(Pr-n)S(O)Pr-n |
| CH₂C(O)OMe | CH(Pr-n)S(O)₂Pr-n |
| CH₂C(O)OMe | CH(Pr-n)SPr-i |
| CH₂C(O)OMe | CH(Pr-n)S(O)Pr-i |
| CH₂C(O)OMe | CH(Pr-n)S(O)₂Pr-i |
| CH₂C(O)OMe | CH(Pr-n)SPr-c |
| CH₂C(O)OMe | CH(Pr-n)S(O)Pr-c |
| CH₂C(O)OMe | CH(Pr-n)S(O)₂Pr-c |
| CH₂C(O)OMe | CH(Pr-n)SCH₂CF₃ |
| CH₂C(O)OMe | CH(Pr-n)S(O)CH₂CF₃ |
| CH₂C(O)OMe | CH(Pr-n)S(O)₂CH₂CF₃ |
| CH₂C(O)OMe | CH(Pr-i)SMe |
| CH₂C(O)OMe | CH(Pr-i)S(O)Me |
| CH₂C(O)OMe | CH(Pr-i)S(O)₂Me |
| CH₂C(O)OMe | CH(Pr-i)SEt |
| CH₂C(O)OMe | CH(Pr-i)S(O)Et |
| CH₂C(O)OMe | CH(Pr-i)S(O)₂Et |
| CH₂C(O)OMe | CH(Pr-i)S(═NCN)Et |
| CH₂C(O)OMe | CH(Pr-i)S(O)(═NCN)Et |
| CH₂C(O)OMe | CH(Pr-i)SPr-n |
| CH₂C(O)OMe | CH(Pr-i)S(O)Pr-n |
| CH₂C(O)OMe | CH(Pr-i)S(O)₂Pr-n |
| CH₂C(O)OMe | CH(Pr-i)SPr-i |
| CH₂C(O)OMe | CH(Pr-i)S(O)Pr-i |
| CH₂C(O)OMe | CH(Pr-i)S(O)₂Pr-i |
| CH₂C(O)OMe | CH(Pr-i)SPr-c |
| CH₂C(O)OMe | CH(Pr-i)S(O)Pr-c |
| CH₂C(O)OMe | CH(Pr-i)S(O)₂Pr-c |
| CH₂C(O)OMe | CH(Pr-i)SCH₂CF₃ |
| CH₂C(O)OMe | CH(Pr-i)S(O)CH₂CF₃ |
| CH₂C(O)OMe | CH(Pr-i)S(O)₂CH₂CF₃ |
| CH₂C(O)OMe | CH(Pr-c)SMe |
| CH₂C(O)OMe | CH(Pr-c)S(O)Me |
| CH₂C(O)OMe | CH(Pr-c)S(O)₂Me |
| CH₂C(O)OMe | CH(Pr-c)SEt |
| CH₂C(O)OMe | CH(Pr-c)S(O)Et |
| CH₂C(O)OMe | CH(Pr-c)S(O)₂Et |
| CH₂C(O)OMe | CH(Pr-c)S(═NCN)Et |
| CH₂C(O)OMe | CH(Pr-c)S(O)(═NCN)Et |
| CH₂C(O)OMe | CH(Pr-c)SPr-n |
| CH₂C(O)OMe | CH(Pr-c)S(O)Pr-n |
| CH₂C(O)OMe | CH(Pr-c)S(O)₂Pr-n |
| CH₂C(O)OMe | CH(Pr-c)SPr-i |
| CH₂C(O)OMe | CH(Pr-c)S(O)Pr-i |
| CH₂C(O)OMe | CH(Pr-c)S(O)₂Pr-i |
| CH₂C(O)OMe | CH(Pr-c)SPr-c |
| CH₂C(O)OMe | CH(Pr-c)S(O)Pr-c |
| CH₂C(O)OMe | CH(Pr-c)S(O)₂Pr-c |
| CH₂C(O)OMe | CH(Pr-c)SCH₂CF₃ |
| CH₂C(O)OMe | CH(Pr-c)S(O)CH₂CF₃ |
| CH₂C(O)OMe | CH(Pr-c)S(O)₂CH₂CF₃ |
| CH₂C(O)OMe | CH(Bu-n)SMe |
| CH₂C(O)OMe | CH(Bu-n)S(O)Me |
| CH₂C(O)OMe | CH(Bu-n)S(O)₂Me |
| CH₂C(O)OMe | CH(Bu-n)SEt |
| CH₂C(O)OMe | CH(Bu-n)S(O)Et |
| CH₂C(O)OMe | CH(Bu-n)S(O)₂Et |
| CH₂C(O)OMe | CH(Bu-n)S(═NCN)Et |
| CH₂C(O)OMe | CH(Bu-n)S(O)(═NCN)Et |
| CH₂C(O)OMe | CH(Bu-n)SPr-n |
| CH₂C(O)OMe | CH(Bu-n)S(O)Pr-n |
| CH₂C(O)OMe | CH(Bu-n)S(O)₂Pr-n |
| CH₂C(O)OMe | CH(Bu-n)SPr-i |
| CH₂C(O)OMe | CH(Bu-n)S(O)Pr-i |
| CH₂C(O)OMe | CH(Bu-n)S(O)₂Pr-i |
| CH₂C(O)OMe | CH(Bu-n)SPr-c |
| CH₂C(O)OMe | CH(Bu-n)S(O)Pr-c |
| CH₂C(O)OMe | CH(Bu-n)S(O)₂Pr-c |
| CH₂C(O)OMe | CH(Bu-n)SCH₂CF₃ |
| CH₂C(O)OMe | CH(Bu-n)S(O)CH₂CF₃ |
| CH₂C(O)OMe | CH(Bu-n)S(O)₂CH₂CF₃ |
| CH₂C(O)OMe | CH(F)SMe |
| CH₂C(O)OMe | CH(F)S(O)Me |
| CH₂C(O)OMe | CH(F)S(O)₂Me |
| CH₂C(O)OMe | CH(F)SEt |
| CH₂C(O)OMe | CH(F)S(O)Et |
| CH₂C(O)OMe | CH(F)S(O)₂Et |
| CH₂C(O)OMe | CH(F)S(═NCN)Et |
| CH₂C(O)OMe | CH(F)S(O)(═NCN)Et |
| CH₂C(O)OMe | CH(F)SCH₂CF₃ |
| CH₂C(O)OMe | CH(F)S(O)CH₂CF₃ |
| CH₂C(O)OMe | CH(F)S(O)₂CH₂CF₃ |
| CH₂C(O)OMe | C(F)₂SMe |
| CH₂C(O)OMe | C(F)₂S(O)Me |
| CH₂C(O)OMe | C(F)₂S(O)₂Me |
| CH₂C(O)OMe | C(F)₂SEt |
| CH₂C(O)OMe | C(F)₂S(O)Et |
| CH₂C(O)OMe | C(F)₂S(O)₂Et |
| CH₂C(O)OMe | C(F)₂S(═NCN)Et |
| CH₂C(O)OMe | C(F)₂S(O)(═NCN)Et |
| CH₂C(O)OMe | C(F)₂SCH₂CF₃ |
| CH₂C(O)OMe | C(F)₂S(O)CH₂CF₃ |
| CH₂C(O)OMe | C(F)₂S(O)₂CH₂CF₃ |
| CH₂C(O)OMe | CH(Cl)SMe |
| CH₂C(O)OMe | CH(Cl)S(O)Me |
| CH₂C(O)OMe | CH(Cl)S(O)₂Me |
| CH₂C(O)OMe | CH(Cl)SEt |
| CH₂C(O)OMe | CH(Cl)S(O)Et |
| CH₂C(O)OMe | CH(Cl)S(O)₂Et |
| CH₂C(O)OMe | CH(Cl)S(═NCN)Et |
| CH₂C(O)OMe | CH(Cl)S(O)(═NCN)Et |
| CH₂C(O)OMe | CH(Cl)SCH₂CF₃ |
| CH₂C(O)OMe | CH(Cl)S(O)CH₂CF₃ |
| CH₂C(O)OMe | CH(Cl)S(O)₂CH₂CF₃ |
| CH₂C(O)OMe | C(Cl)₂SMe |
| CH₂C(O)OMe | C(Cl)₂S(O)Me |
| CH₂C(O)OMe | C(Cl)₂S(O)₂Me |
| CH₂C(O)OMe | C(Cl)₂SEt |
| CH₂C(O)OMe | C(Cl)₂S(O)Et |
| CH₂C(O)OMe | C(Cl)₂S(O)₂Et |
| CH₂C(O)OMe | C(Cl)₂S(═NCN)Et |
| CH₂C(O)OMe | C(Cl)₂S(O)(═NCN)Et |
| CH₂C(O)OMe | C(Cl)₂SCH₂CF₃ |
| CH₂C(O)OMe | C(Cl)₂S(O)CH₂CF₃ |
| CH₂C(O)OMe | C(Cl)₂S(O)₂CH₂CF₃ |
| CH₂C(O)OMe | CH(Br)SMe |
| CH₂C(O)OMe | CH(Br)S(O)Me |
| CH₂C(O)OMe | CH(Br)S(O)₂Me |
| CH₂C(O)OMe | CH(Br)SEt |
| CH₂C(O)OMe | CH(Br)S(O)Et |
| CH₂C(O)OMe | CH(Br)S(O)₂Et |
| CH₂C(O)OMe | CH(Br)S(═NCN)Et |
| CH₂C(O)OMe | CH(Br)S(O)(═NCN)Et |
| CH₂C(O)OMe | CH(Br)SCH₂CF₃ |
| CH₂C(O)OMe | CH(Br)S(O)CH₂CF₃ |
| CH₂C(O)OMe | CH(Br)S(O)₂CH₂CF₃ |
| CH₂C(O)OMe | CH(I)SMe |
| CH₂C(O)OMe | CH(I)S(O)Me |
| CH₂C(O)OMe | CH(I)S(O)₂Me |
| CH₂C(O)OMe | CH(I)SEt |
| CH₂C(O)OMe | CH(I)S(O)Et |
| CH₂C(O)OMe | CH(I)S(O)₂Et |
| CH₂C(O)OMe | CH(I)S(═NCN)Et |
| CH₂C(O)OMe | CH(I)S(O)(═NCN)Et |
| CH₂C(O)OMe | CH(I)SCH₂CF₃ |
| CH₂C(O)OMe | CH(I)S(O)CH₂CF₃ |
| CH₂C(O)OMe | CH(I)S(O)₂CH₂CF₃ |
| CH₂C(O)OMe | CH{OC(O)Me}SMe |
| CH₂C(O)OMe | CH{OC(O)Me}S(O)Me |
| CH₂C(O)OMe | CH{OC(O)Me}S(O)₂Me |
| CH₂C(O)OMe | CH{OC(O)OMe}SEt |
| CH₂C(O)OMe | CH{OC(O)OMe}S(O)Et |
| CH₂C(O)OMe | CH{OC(O)OMe}S(O)₂Et |
| CH₂C(O)OMe | CH{OC(O)OMe}S(═NCN)Et |
| CH₂C(O)OMe | CH{OC(O)OMe}S(O)(═NCN)Et |
| CH₂C(O)OMe | CH{OC(O)OEt}SMe |
| CH₂C(O)OMe | CH{OC(O)OEt}S(O)Me |
| CH₂C(O)OMe | CH{OC(O)OEt}S(O)₂Me |
| CH₂C(O)OMe | CH{OC(O)OEt}SEt |
| CH₂C(O)OMe | CH{OC(O)OEt}S(O)Et |
| CH₂C(O)OMe | CH{OC(O)OEt}S(O)₂Et |
| CH₂C(O)OMe | CH{OC(O)OEt}S(═NCN)Et |
| CH₂C(O)OMe | CH{OC(O)OEt}S(O)(═NCN)Et |
| CH₂C(O)OMe | CH(OMe)SMe |
| CH₂C(O)OMe | CH(OMe)S(O)Me |
| CH₂C(O)OMe | CH(OMe)S(O)₂Me |
| CH₂C(O)OMe | CH(OMe)SEt |
| CH₂C(O)OMe | CH(OMe)S(O)Et |
| CH₂C(O)OMe | CH(OMe)S(O)₂Et |
| CH₂C(O)OMe | CH(OMe)S(═NCN)Et |
| CH₂C(O)OMe | CH(OMe)S(O)(═NCN)Et |
| CH₂C(O)OMe | CH(OEt)SMe |
| CH₂C(O)OMe | CH(OEt)S(O)Me |
| CH₂C(O)OMe | CH(OEt)S(O)₂Me |
| CH₂C(O)OMe | CH(OEt)SEt |
| CH₂C(O)OMe | CH(OEt)S(O)Et |
| CH₂C(O)OMe | CH(OEt)S(O)₂Et |
| CH₂C(O)OMe | CH(OEt)S(═NCN)Et |
| CH₂C(O)OMe | CH(OEt)S(O)(═CN)Et |
| CH₂C(O)OMe | CH(SMe)₂ |
| CH₂C(O)OMe | CH(SMe)S(O)Me |
| CH₂C(O)OMe | CH(SMe)S(O)₂Me |
| CH₂C(O)OMe | CH(SMe)SEt |
| CH₂C(O)OMe | CH(SMe)S(O)Et |
| CH₂C(O)OMe | CH(SMe)S(O)₂Et |
| CH₂C(O)OMe | CH(SMe)S(═NCN)Et |
| CH₂C(O)OMe | CH(SMe)S(O)(═NCN)Et |
| CH₂C(O)OMe | CH(SEt)SMe |
| CH₂C(O)OMe | CH(SEt)S(O)Me |
| CH₂C(O)OMe | CH(SEt)S(O)₂Me |
| CH₂C(O)OMe | CH(SEt)SEt |
| CH₂C(O)OMe | CH(SEt)S(O)Et |
| CH₂C(O)OMe | CH(SEt)S(O)₂Et |
| CH₂C(O)OMe | CH(SEt)S(═NCN)Et |
| CH₂C(O)OMe | CH(SEt)S(O)(═NCN)Et |
| CH₂C(O)OMe | CH(CN)SMe |
| CH₂C(O)OMe | CH(CN)S(O)Me |
| CH₂C(O)OMe | CH(CN)S(O)₂Me |
| CH₂C(O)OMe | CH(CN)SEt |
| CH₂C(O)OMe | CH(CN)S(O)Et |
| CH₂C(O)OMe | CH(CN)S(O)₂Et |
| CH₂C(O)OMe | CH(CN)S(═NCN)Et |
| CH₂C(O)OMe | CH(CN)S(O)(═NCN)Et |
| CH₂C(O)OMe | CH{C(O)OMe}SMe |
| CH₂C(O)OMe | CH{C(O)OMe}S(O)Me |
| CH₂C(O)OMe | CH{C(O)OMe}S(O)₂Me |
| CH₂C(O)OMe | CH{C(O)OMe}SMe |
| CH₂C(O)OMe | CH{C(O)OMe}S(O)Me |
| CH₂C(O)OMe | CH{C(O)OMe}S(O)₂Me |
| CH₂C(O)OMe | CH{C(O)OMe}SEt |
| CH₂C(O)OMe | CH{C(O)OMe}S(O)Et |
| CH₂C(O)OMe | CH{C(O)OMe}S(O)₂Et |
| CH₂C(O)OMe | CH{C(O)OMe}S(═NCN)Et |
| CH₂C(O)OMe | CH{C(O)OMe}S(O)(═NCN)Et |
| CH₂C(O)OMe | CH{C(O)OEt}SMe |
| CH₂C(O)OMe | CH{C(O)OEt}S(O)Me |
| CH₂C(O)OMe | CH{C(O)OEt}S(O)₂Me |
| CH₂C(O)OMe | CH{C(O)OEt}SEt |
| CH₂C(O)OMe | CH{C(O)OEt}S(O)Et |
| CH₂C(O)OMe | CH{C(O)OEt}S(O)₂Et |
| CH₂C(O)OMe | CH{C(O)OEt}S(═NCN)Et |
| CH₂C(O)OMe | CH{C(O)OEt}S(O)(═NCN)Et |
| CH₂C(O)OMe | C(Me)₂SMe |
| CH₂C(O)OMe | C(Me)₂S(O)Me |
| CH₂C(O)OMe | C(Me)₂S(O)₂Me |
| CH₂C(O)OMe | CH₂CH₂SMe |
| CH₂C(O)OMe | CH₂CH₂S(O)Me |
| CH₂C(O)OMe | CH₂CH₂S(O)₂Me |
| CH₂C(O)OMe | CH(Me)CH₂SMe |
| CH₂C(O)OMe | CH(Me)CH₂S(O)Me |
| CH₂C(O)OMe | CH(Me)CH₂S(O)₂Me |
| CH₂C(O)OMe | NHPh |
| CH₂C(O)OMe | NH(D1-1a) |
| CH₂C(O)OMe | NH(D1-2a) |
| CH₂C(O)OMe | NH(D1-2b) |
| CH₂C(O)OMe | NH{D1-4b(X^{1a}═Me)} |
| CH₂C(O)OMe | NH(D1-5a) |
| CH₂C(O)OMe | NH(D1-5b) |
| CH₂C(O)OMe | NH(D1-5c) |
| CH₂C(O)OMe | NH(D1-6a) |
| CH₂C(O)OMe | NH(D1-6b) |
| CH₂C(O)OMe | NH(D1-6c) |
| CH₂C(O)OMe | NH(D1-7a) |
| CH₂C(O)OMe | NH{D1-8a(X^{1a}═Me)} |
| CH₂C(O)OMe | NH{D1-8b(X^{1a}═Me)} |
| CH₂C(O)OMe | NH{D1-8c(X^{1a}═Me)} |
| CH₂C(O)OMe | NH(D1-9a) |
| CH₂C(O)OMe | NH(D1-9b) |
| CH₂C(O)OMe | NH(D1-9c) |
| CH₂C(O)OMe | NH(D1-10a) |
| CH₂C(O)OMe | NH(D1-10b) |
| CH₂C(O)OMe | NH(D1-10c) |
| CH₂C(O)OMe | NH(D1-11a) |
| CH₂C(O)OMe | NH{D1-12a(X^{1a}═Me)} |
| CH₂C(O)OMe | NH{D1-12b(X^{1a}═Me)} |
| CH₂C(O)OMe | NH{D1-12c(X^{1a}═Me)} |
| CH₂C(O)OMe | NH(D1-13a) |
| CH₂C(O)OMe | NH(D1-13b) |
| CH₂C(O)OMe | NH(D1-14a) |
| CH₂C(O)OMe | NH(D1-14b) |
| CH₂C(O)OMe | NH(D1-15a) |
| CH₂C(O)OMe | NH(D1-15b) |
| CH₂C(O)OMe | NH(D1-16a) |
| CH₂C(O)OMe | NH(D1-17a) |
| CH₂C(O)OMe | NH(D1-18a) |
| CH₂C(O)OMe | NH(D1-19a) |
| CH₂C(O)OMe | NH(D1-20a) |
| CH₂C(O)OMe | NH{D-21a(X^{1a}═Me)} |
| CH₂C(O)OMe | NH{D1-21b(X^{1a}═Me)} |
| CH₂C(O)OMe | NH(D1-22a) |
| CH₂C(O)OMe | NH{D1-23a(X^{1a}═Me)} |
| CH₂C(O)OMe | NH{D1-23b(X^{1a}═Me)} |
| CH₂C(O)OMe | NH(D1-24a) |
| CH₂C(O)OMe | NH{D1-25a(X^{1a}═Me)} |
| CH₂C(O)OMe | NH(D1-26a) |
| CH₂C(O)OMe | NH{D1-27a(X^{1a}═Me)} |
| CH₂C(O)OMe | NH(D1-28a) |
| CH₂C(O)OMe | NH(D1-29a) |
| CH₂C(O)OMe | NH{D1-30a(X^{1a}═Me)} |
| CH₂C(O)OMe | NH{D1-31a(X^{1a}═Me)} |
| CH₂C(O)OMe | NH(D1-32a) |
| CH₂C(O)OMe | NH(D1-33a) |
| CH₂C(O)OMe | NH(D1-34a) |
| CH₂C(O)OMe | NH(D1-35a) |
| CH₂C(O)OMe | NH(D1-35b) |
| CH₂C(O)OMe | NH(D1-36a) |
| CH₂C(O)OMe | NH(D1-37a) |
| CH₂C(O)OMe | NH(D1-38a) |
| CH₂C(O)OMe | NH(D1-39a) |
| CH₂C(O)OMe | NH(D1-40a) |
| CH₂C(O)OMe | NH(D1-40b) |
| CH₂C(O)OMe | NH(D1-40c) |
| CH₂C(O)OMe | NH(D1-41a) |
| CH₂C(O)OMe | NH(D1-42a) |
| CH₂C(O)OMe | NH(D1-43a) |
| CH₂C(O)OMe | NH(D1-45a) |
| CH₂C(O)OMe | NH(D1-49a) |
| CH₂C(O)OMe | NH(D1-51a) |
| CH₂C(O)OMe | NH(D1-59a) |
| CH₂C(O)OMe | NH(D1-61a) |
| CH₂C(O)OMe | NH(D1-79a) |
| CH₂C(O)OMe | NH(D1-80a) |
| CH₂C(O)OMe | NH{D1-108b(2-Cl)} |
| CH₂C(O)OMe | NH{D1-108b(3-Cl)} |
| CH₂C(O)OMe | NH{D1-108b(4-Cl)} |
| CH₂C(O)OMe | NH{D1-108b(2-Me)} |
| CH₂C(O)OMe | NH{D1-108b(3-Me)} |
| CH₂C(O)OMe | NH{D1-108b(4-Me)} |
| CH₂C(O)OMe | N(Me)Ph |
| CH₂C(O)OMe | N(Me)(D1-1a) |
| CH₂C(O)OMe | N(Me)(D1-2a) |
| CH₂C(O)OMe | N(Me)(D1-2b) |
| CH₂C(O)OMe | N(Me){D1-4a(X^{1a}═Me)} |
| CH₂C(O)OMe | N(Me){D1-4b(X^{1a}═Me)} |
| CH₂C(O)OMe | N(Me)(D1-5a) |
| CH₂C(O)OMe | N(Me)(D1-5b) |
| CH₂C(O)OMe | N(Me)(D1-5c) |
| CH₂C(O)OMe | N(Me)(D1-6a) |
| CH₂C(O)OMe | N(Me)(D1-6b) |
| CH₂C(O)OMe | N(Me)(D1-6c) |
| CH₂C(O)OMe | N(Me)(D1-7a) |
| CH₂C(O)OMe | N(Me){D1-8a(X^{1a}═Me)} |
| CH₂C(O)OMe | N(Me){D1-8b(X^{1a}═Me)} |
| CH₂C(O)OMe | N(Me){D1-8c(X^{1a}═Me)} |
| CH₂C(O)OMe | N(Me)(D1-9a) |
| CH₂C(O)OMe | N(Me)(D1-9b) |
| CH₂C(O)OMe | N(Me)(D1-9c) |
| CH₂C(O)OMe | N(Me)(D1-10a) |
| CH₂C(O)OMe | N(Me)(D1-10b) |
| CH₂C(O)OMe | N(Me)(D1-10c) |
| CH₂C(O)OMe | N(Me)(D1-11a) |
| CH₂C(O)OMe | N(Me){D1-12a(X^{1a}═Me)} |
| CH₂C(O)OMe | N(Me){D1-12b(X^{1a}═Me)} |
| CH₂C(O)OMe | N(Me){D1-12c(X^{1a}═Me)} |
| CH₂C(O)OMe | N(Me)(D1-13a) |
| CH₂C(O)OMe | N(Me)(D1-13b) |
| CH₂C(O)OMe | N(Me)(D1-14a) |
| CH₂C(O)OMe | N(Me)(D1-14b) |
| CH₂C(O)OMe | N(Me)(D1-15a) |
| CH₂C(O)OMe | N(Me)(D1-15b) |
| CH₂C(O)OMe | N(Me)(D1-16a) |
| CH₂C(O)OMe | N(Me)(D1-17a) |
| CH₂C(O)OMe | N(Me)(D1-18a) |
| CH₂C(O)OMe | N(Me)(D1-19a) |
| CH₂C(O)OMe | N(Me)(D1-20a) |
| CH₂C(O)OMe | N(Me){D1-21a(X^{1a}═Me)} |
| CH₂C(O)OMe | N(Me){D1-21b(X^{1a}═Me)} |
| CH₂C(O)OMe | N(Me)(D1-22a) |
| CH₂C(O)OMe | N(Me){D1-23a(X^{1a}═Me)} |
| CH₂C(O)OMe | N(Me){D1-23b(X^{1a}═Me)} |
| CH₂C(O)OMe | N(Me)(D1-24a) |
| CH₂C(O)OMe | N(Me){D1-25a(X^{1a}═Me)} |
| CH₂C(O)OMe | N(Me)(D1-26a) |
| CH₂C(O)OMe | N(Me){D1-27a(X^{1a}═Me)} |
| CH₂C(O)OMe | N(Me)(D1-28a) |
| CH₂C(O)OMe | N(Me)(D1-29a) |
| CH₂C(O)OMe | N(Me){D1-30a(X^{1a}═Me)} |
| CH₂C(O)OMe | N(Me){D1-31a(X^{1a}═Me)} |
| CH₂C(O)OMe | N(Me)(D1-32a) |
| CH₂C(O)OMe | N(Me)(D1-34a) |
| CH₂C(O)OMe | N(Me)(D1-35a) |
| CH₂C(O)OMe | N(Me)(D1-35b) |
| CH₂C(O)OMe | N(Me)(D1-36a) |
| CH₂C(O)OMe | N(Me)(D1-37a) |
| CH₂C(O)OMe | N(Me)(D1-38a) |
| CH₂C(O)OMe | N(Me)(D1-39a) |
| CH₂C(O)OMe | N(Me)(D1-40a) |
| CH₂C(O)OMe | N(Me)(D1-40b) |
| CH₂C(O)OMe | N(Me)(D1-40c) |
| CH₂C(O)OMe | N(Me)(D1-41a) |
| CH₂C(O)OMe | N(Me)(D1-42a) |
| CH₂C(O)OMe | N(Me)(D1-43a) |
| CH₂C(O)OMe | N(Me)(D1-45a) |
| CH₂C(O)OMe | N(Me)(D1-49a) |
| CH₂C(O)OMe | N(Me)(D1-51a) |
| CH₂C(O)OMe | N(Me)(D1-59a) |
| CH₂C(O)OMe | N(Me)(D1-61a) |
| CH₂C(O)OMe | N(Me)(D1-79a) |
| CH₂C(O)OMe | N(Me)(D1-80a) |
| CH₂C(O)OMe | N(Me){D1-108b(2-Cl)} |
| CH₂C(O)OMe | N(Me){D1-108b(3-Cl)} |
| CH₂C(O)OMe | N(Me){D1-108b(4-Cl)} |
| CH₂C(O)OMe | N(Me){D1-108b(2-Me)} |
| CH₂C(O)OMe | N(Me){D1-108b(3-Me)} |
| CH₂C(O)OMe | N(Me){D1-108b(4-Me)} |
| CH₂C(O)OEt | CH₂SMe |
| CH₂C(O)OEt | CH₂S(O)Me |
| CH₂C(O)OEt | CH₂S(O)₂Me |
| CH₂C(O)OEt | CH₂SEt |
| CH₂C(O)OEt | CH₂S(O)Et |
| CH₂C(O)OEt | CH₂S(O)₂Et |
| CH₂C(O)OEt | CH₂SPr-n |
| CH₂C(O)OEt | CH₂S(O)Pr-n |
| CH₂C(O)OEt | CH₂S(O)₂Pr-n |
| CH₂C(O)OEt | CH₂SPr-i |
| CH₂C(O)OEt | CH₂S(O)Pr-i |
| CH₂C(O)OEt | CH₂S(O)₂Pr-i |
| CH₂C(O)OEt | CH₂SPr-c |
| CH₂C(O)OEt | CH₂S(O)Pr-c |
| CH₂C(O)OEt | CH₂S(O)₂Pr-c |
| CH₂C(O)OEt | CH₂SCH₂CH═CH₂ |
| CH₂C(O)OEt | CH₂S(O)CH₂CH═CH₂ |
| CH₂C(O)OEt | CH₂S(O)₂CH₂CH═CH₂ |
| CH₂C(O)OEt | CH₂SCH₂C≡CH |
| CH₂C(O)OEt | CH₂S(O)CH₂C≡CH |
| CH₂C(O)OEt | CH₂S(O)₂CH₂C≡CH |
| CH₂C(O)OEt | CH₂SCH₂CF₃ |
| CH₂C(O)OEt | CH₂S(O)CH₂CF₃ |
| CH₂C(O)OEt | CH₂S(O)₂CH₂CF₃ |
| CH₂C(O)OEt | CH₂SCH₂CH(OMe)₂ |
| CH₂C(O)OEt | CH₂SCH₂CH(═NOMe) |
| CH₂C(O)OEt | CH(Me)SMe |
| CH₂C(O)OEt | CH(Me)S(O)Me |
| CH₂C(O)OEt | CH(Me)S(O)₂Me |
| CH₂C(O)OEt | CH(Me)SEt |
| CH₂C(O)OEt | CH(Me)S(O)Et |
| CH₂C(O)OEt | CH(Me)S(O)₂Et |
| CH₂C(O)OEt | CH(Me)S(═NCN)Et |
| CH₂C(O)OEt | CH(Me)S(O)(═NCN)Et |
| CH₂C(O)OEt | CH(Me)SPr-n |
| CH₂C(O)OEt | CH(Me)S(O)Pr-n |
| CH₂C(O)OEt | CH(Me)S(O)₂Pr-n |
| CH₂C(O)OEt | CH(Me)SPr-i |
| CH₂C(O)OEt | CH(Me)S(O)Pr-i |
| CH₂C(O)OEt | CH(Me)S(O)₂Pr-i |
| CH₂C(O)OEt | CH(Me)SBu-t |
| CH₂C(O)OEt | CH(Me)S(O)Bu-t |
| CH₂C(O)OEt | CH(Me)S(O)₂Bu-t |
| CH₂C(O)OEt | CH(Me)SCH₂Pr-c |
| CH₂C(O)OEt | CH(Me)S(O)CH₂Pr-c |
| CH₂C(O)OEt | CH(Me)S(O)₂CH₂Pr-c |
| CH₂C(O)OEt | CH(Me)SCH₂OMe |
| CH₂C(O)OEt | CH(Me)SCH₂SMe |
| CH₂C(O)OEt | CH(Me)S(O)CH₂SMe |
| CH₂C(O)OEt | CH(Me)S(O)₂CH₂SMe |
| CH₂C(O)OEt | CH(Me)SCH₂C═CH₂ |
| CH₂C(O)OEt | CH(Me)S(O)CH₂C═CH₂ |
| CH₂C(O)OEt | CH(Me)S(O)₂CH₂C═CH₂ |
| CH₂C(O)OEt | CH(Me)SCH₂C≡CH |
| CH₂C(O)OEt | CH(Me)S(O)CH₂C≡CH |
| CH₂C(O)OEt | CH(Me)S(O)₂CH₂C≡CH |
| CH₂C(O)OEt | CH(Me)SCH₂C(O)NHMe |
| CH₂C(O)OEt | CH(Me)S(O)CH₂C(O)NHMe |
| CH₂C(O)OEt | CH(Me)S(O)₂CH₂C(O)NHMe |
| CH₂C(O)OEt | CH(Me)SCH₂C(O)OMe |
| CH₂C(O)OEt | CH(Me)S(O)CH₂C(O)OMe |
| CH₂C(O)OEt | CH(Me)S(O)₂CH₂C(O)OMe |
| CH₂C(O)OEt | CH(Me)SCH₂CH₂CH₂Cl |
| CH₂C(O)OEt | CH(Me)S(O)CH₂CH₂CH₂Cl |
| CH₂C(O)OEt | CH(Me)S(O)₂CH₂CH₂CH₂Cl |
| CH₂C(O)OEt | CH(Me)SCH₂CF₃ |
| CH₂C(O)OEt | CH(Me)S(O)CH₂CF₃ |
| CH₂C(O)OEt | CH(Me)S(O)₂CH₂CF₃ |
| CH₂C(O)OEt | CH(Me)SCH₂Ph |
| CH₂C(O)OEt | CH(Me)S(O)CH₂Ph |
| CH₂C(O)OEt | CH(Me)S(O)₂CH₂Ph |
| CH₂C(O)OEt | CH(Me)SCH₂(D1-34a) |
| CH₂C(O)OEt | CH(Me)S(O)CH₂(D1-34a) |
| CH₂C(O)OEt | CH(Me)S(O)₂CH₂(D1-34a) |
| CH₂C(O)OEt | CH(CH₃)SCH₂Si(CH₃)₃ |
| CH₂C(O)OEt | CH(Me)SCN |
| CH₂C(O)OEt | CH(Me)SCH₂CN |
| CH₂C(O)OEt | CH(Me)SC(O)Me |
| CH₂C(O)OEt | CH(Me)S{D1-12a(X^{1a}═Me)} |
| CH₂C(O)OEt | CH(Me)S(D1-32a) |
| CH₂C(O)OEt | CH(Me)S{D1-32b(3-NO₂)} |
| CH₂C(O)OEt | CH(Me)S{D1-32b(3-CF₃)} |
| CH₂C(O)OEt | CH(Me)S(D1-37a) |
| CH₂C(O)OEt | CH(Me)S(D1-51a) |
| CH₂C(O)OEt | CH(Et)SMe |
| CH₂C(O)OEt | CH(Et)S(O)Me |
| CH₂C(O)OEt | CH(Et)S(O)₂Me |
| CH₂C(O)OEt | CH(Et)SEt |
| CH₂C(O)OEt | CH(Et)S(O)Et |
| CH₂C(O)OEt | CH(Et)S(O)₂Et |
| CH₂C(O)OEt | CH(Et)SCH₂CF₃ |
| CH₂C(O)OEt | CH(Et)S(O)CH₂CF₃ |
| CH₂C(O)OEt | CH(Et)S(O)₂CH₂CF₃ |
| CH₂C(O)OEt | CH(Pr-n)SMe |
| CH₂C(O)OEt | CH(Pr-n)S(O)Me |
| CH₂C(O)OEt | CH(Pr-n)S(O)₂Me |
| CH₂C(O)OEt | CH(Pr-n)SEt |
| CH₂C(O)OEt | CH(Pr-n)S(O)Et |
| CH₂C(O)OEt | CH(Pr-n)S(O)₂Et |
| CH₂C(O)OEt | CH(Pr-n)S(═NCN)Et |
| CH₂C(O)OEt | CH(Pr-n)S(O)(═NCN)Et |
| CH₂C(O)OEt | CH(Pr-n)SPr-n |
| CH₂C(O)OEt | CH(Pr-n)S(O)Pr-n |
| CH₂C(O)OEt | CH(Pr-n)S(O)₂Pr-n |
| CH₂C(O)OEt | CH(Pr-n)SPr-i |
| CH₂C(O)OEt | CH(Pr-n)S(O)Pr-i |
| CH₂C(O)OEt | CH(Pr-n)S(O)₂Pr-i |
| CH₂C(O)OEt | CH(Pr-n)SPr-c |
| CH₂C(O)OEt | CH(Pr-n)S(O)Pr-c |
| CH₂C(O)OEt | CH(Pr-n)S(O)₂Pr-c |
| CH₂C(O)OEt | CH(Pr-n)SCH₂CF₃ |
| CH₂C(O)OEt | CH(Pr-n)S(O)CH₂CF₃ |
| CH₂C(O)OEt | CH(Pr-n)S(O)₂CH₂CF₃ |
| CH₂C(O)OEt | CH(Pr-i)SMe |
| CH₂C(O)OEt | CH(Pr-i)S(O)Me |
| CH₂C(O)OEt | CH(Pr-i)S(O)₂Me |
| CH₂C(O)OEt | CH(Pr-i)SEt |
| CH₂C(O)OEt | CH(Pr-i)S(O)Et |
| CH₂C(O)OEt | CH(Pr-i)S(O)₂Et |
| CH₂C(O)OEt | CH(Pr-i)S(═NCN)Et |
| CH₂C(O)OEt | CH(Pr-i)S(O)(═NCN)Et |
| CH₂C(O)OEt | CH(Pr-i)SPr-n |
| CH₂C(O)OEt | CH(Pr-i)S(O)Pr-n |
| CH₂C(O)OEt | CH(Pr-i)S(O)₂Pr-n |
| CH₂C(O)OEt | CH(Pr-i)SPr-i |
| CH₂C(O)OEt | CH(Pr-i)S(O)Pr-i |
| CH₂C(O)OEt | CH(Pr-i)S(O)₂Pr-i |
| CH₂C(O)OEt | CH(Pr-i)SPr-c |
| CH₂C(O)OEt | CH(Pr-i)S(O)Pr-c |
| CH₂C(O)OEt | CH(Pr-i)S(O)₂Pr-c |
| CH₂C(O)OEt | CH(Pr-i)SCH₂CF₃ |
| CH₂C(O)OEt | CH(Pr-i)S(O)CH₂CF₃ |
| CH₂C(O)OEt | CH(Pr-i)S(O)₂CH₂CF₃ |
| CH₂C(O)OEt | CH(Pr-c)SMe |
| CH₂C(O)OEt | CH(Pr-c)S(O)Me |
| CH₂C(O)OEt | CH(Pr-c)S(O)₂Me |
| CH₂C(O)OEt | CH(Pr-c)SEt |
| CH₂C(O)OEt | CH(Pr-c)S(O)Et |
| CH₂C(O)OEt | CH(Pr-c)S(O)₂Et |
| CH₂C(O)OEt | CH(Pr-c)S(═NCN)Et |
| CH₂C(O)OEt | CH(Pr-c)S(O)(═NCN)Et |
| CH₂C(O)OEt | CH(Pr-c)SPr-n |
| CH₂C(O)OEt | CH(Pr-c)S(O)Pr-n |
| CH₂C(O)OEt | CH(Pr-c)S(O)₂Pr-n |
| CH₂C(O)OEt | CH(Pr-c)SPr-i |
| CH₂C(O)OEt | CH(Pr-c)S(O)Pr-i |
| CH₂C(O)OEt | CH(Pr-c)S(O)₂Pr-i |
| CH₂C(O)OEt | CH(Pr-c)SPr-c |
| CH₂C(O)OEt | CH(Pr-c)S(O)Pr-c |
| CH₂C(O)OEt | CH(Pr-c)S(O)₂Pr-c |
| CH₂C(O)OEt | CH(Pr-c)SCH₂CF₃ |
| CH₂C(O)OEt | CH(Pr-c)S(O)CH₂CF₃ |
| CH₂C(O)OEt | CH(Pr-c)S(O)₂CH₂CF₃ |
| CH₂C(O)OEt | CH(Bu-n)SMe |
| CH₂C(O)OEt | CH(Bu-n)S(O)Me |
| CH₂C(O)OEt | CH(Bu-n)S(O)₂Me |
| CH₂C(O)OEt | CH(Bu-n)SEt |
| CH₂C(O)OEt | CH(Bu-n)S(O)Et |
| CH₂C(O)OEt | CH(Bu-n)S(O)₂Et |
| CH₂C(O)OEt | CH(Bu-n)S(═NCN)Et |
| CH₂C(O)OEt | CH(Bu-n)S(O)(═NCN)Et |
| CH₂C(O)OEt | CH(Bu-n)SPr-n |
| CH₂C(O)OEt | CH(Bu-n)S(O)Pr-n |
| CH₂C(O)OEt | CH(Bu-n)S(O)₂Pr-n |
| CH₂C(O)OEt | CH(Bu-n)SPr-i |
| CH₂C(O)OEt | CH(Bu-n)S(O)Pr-i |
| CH₂C(O)OEt | CH(Bu-n)S(O)₂Pr-i |
| CH₂C(O)OEt | CH(Bu-n)SPr-c |
| CH₂C(O)OEt | CH(Bu-n)S(O)Pr-c |
| CH₂C(O)OEt | CH(Bu-n)S(O)₂Pr-c |
| CH₂C(O)OEt | CH(Bu-n)SCH₂CF₃ |
| CH₂C(O)OEt | CH(Bu-n)S(O)CH₂CF₃ |
| CH₂C(O)OEt | CH(Bu-n)S(O)₂CH₂CF₃ |
| CH₂C(O)OEt | CH(F)SMe |
| CH₂C(O)OEt | CH(F)S(O)Me |
| CH₂C(O)OEt | CH(F)S(O)₂Me |
| CH₂C(O)OEt | CH(F)SEt |
| CH₂C(O)OEt | CH(F)S(O)Et |
| CH₂C(O)OEt | CH(F)S(O)₂Et |
| CH₂C(O)OEt | CH(F)S(═NCN)Et |
| CH₂C(O)OEt | CH(F)S(O)(═NCN)Et |
| CH₂C(O)OEt | CH(F)SCH₂CF₃ |
| CH₂C(O)OEt | CH(F)S(O)CH₂CF₃ |
| CH₂C(O)OEt | CH(F)S(O)₂CH₂CF₃ |
| CH₂C(O)OEt | C(F)₂SMe |
| CH₂C(O)OEt | C(F)₂S(O)Me |
| CH₂C(O)OEt | C(F)₂S(O)₂Me |
| CH₂C(O)OEt | C(F)₂SEt |
| CH₂C(O)OEt | C(F)₂S(O)Et |
| CH₂C(O)OEt | C(F)₂S(O)₂Et |
| CH₂C(O)OEt | C(F)₂S(═NCN)Et |
| CH₂C(O)OEt | C(F)₂S(O)(═NCN)Et |
| CH₂C(O)OEt | C(F)₂SCH₂CF₃ |
| CH₂C(O)OEt | C(F)₂S(O)CH₂CF₃ |
| CH₂C(O)OEt | C(F)₂S(O)₂CH₂CF₃ |
| CH₂C(O)OEt | CH(Cl)SMe |
| CH₂C(O)OEt | CH(Cl)S(O)Me |
| CH₂C(O)OEt | CH(Cl)S(O)₂Me |
| CH₂C(O)OEt | CH(Cl)SEt |
| CH₂C(O)OEt | CH(Cl)S(O)Et |
| CH₂C(O)OEt | CH(Cl)S(O)₂Et |
| CH₂C(O)OEt | CH(Cl)S(═NCN)Et |
| CH₂C(O)OEt | CH(Cl)S(O)(═NCN)Et |
| CH₂C(O)OEt | CH(Cl)SCH₂CF₃ |
| CH₂C(O)OEt | CH(Cl)S(O)CH₂CF₃ |
| CH₂C(O)OEt | CH(Cl)S(O)₂CH₂CF₃ |
| CH₂C(O)OEt | C(Cl)₂SMe |
| CH₂C(O)OEt | C(Cl)₂S(O)Me |
| CH₂C(O)OEt | C(Cl)₂S(O)₂Me |
| CH₂C(O)OEt | C(Cl)₂SEt |
| CH₂C(O)OEt | C(Cl)₂S(O)Et |
| CH₂C(O)OEt | C(Cl)₂S(O)₂Et |
| CH₂C(O)OEt | C(Cl)₂S(═NCN)Et |
| CH₂C(O)OEt | C(Cl)₂S(O)(═NCN)Et |
| CH₂C(O)OEt | C(Cl)₂SCH₂CF₃ |
| CH₂C(O)OEt | C(Cl)₂S(O)CH₂CF₃ |
| CH₂C(O)OEt | C(Cl)₂S(O)₂CH₂CF₃ |
| CH₂C(O)OEt | CH(Br)SMe |
| CH₂C(O)OEt | CH(Br)S(O)Me |
| CH₂C(O)OEt | CH(Br)S(O)₂Me |
| CH₂C(O)OEt | CH(Br)SEt |
| CH₂C(O)OEt | CH(Br)S(O)Et |
| CH₂C(O)OEt | CH(Br)S(O)₂Et |
| CH₂C(O)OEt | CH(Br)S(═NCN)Et |
| CH₂C(O)OEt | CH(Br)S(O)(═NCN)Et |
| CH₂C(O)OEt | CH(Br)SCH₂CF₃ |
| CH₂C(O)OEt | CH(Br)S(O)CH₂CF₃ |
| CH₂C(O)OEt | CH(Br)S(O)₂CH₂CF₃ |
| CH₂C(O)OEt | CH(I)SMe |
| CH₂C(O)OEt | CH(I)S(O)Me |
| CH₂C(O)OEt | CH(I)S(O)₂Me |
| CH₂C(O)OEt | CH(I)SEt |
| CH₂C(O)OEt | CH(I)S(O)Et |
| CH₂C(O)OEt | CH(I)S(O)₂Et |
| CH₂C(O)OEt | CH(I)S(═NCN)Et |
| CH₂C(O)OEt | CH(I)S(O)(═NCN)Et |
| CH₂C(O)OEt | CH(I)SCH₂CF₃ |
| CH₂C(O)OEt | CH(I)S(O)CH₂CF₃ |
| CH₂C(O)OEt | CH(I)S(O)₂CH₂CF₃ |
| CH₂C(O)OEt | CH{OC(O)Me}SMe |
| CH₂C(O)OEt | CH{OC(O)Me}S(O)Me |
| CH₂C(O)OEt | CH{OC(O)Me}S(O)₂Me |
| CH₂C(O)OEt | CH{OC(O)OMe}SEt |
| CH₂C(O)OEt | CH{OC(O)OMe}S(O)Et |
| CH₂C(O)OEt | CH{OC(O)OMe}S(O)₂Et |
| CH₂C(O)OEt | CH{OC(O)OMe}S(═NCN)Et |
| CH₂C(O)OEt | CH{OC(O)OMe}S(O)(═NCN)Et |
| CH₂C(O)OEt | CH{OC(O)OEt}SMe |
| CH₂C(O)OEt | CH{OC(O)OEt}S(O)Me |
| CH₂C(O)OEt | CH{OC(O)OEt}S(O)₂Me |
| CH₂C(O)OEt | CH{OC(O)OEt}SEt |
| CH₂C(O)OEt | CH{OC(O)OEt}S(O)Et |
| CH₂C(O)OEt | CH{OC(O)OEt}S(O)₂Et |
| CH₂C(O)OEt | CH{OC(O)OEt}S(═NCN)Et |
| CH₂C(O)OEt | CH{OC(O)OEt}S(O)(═NCN)Et |
| CH₂C(O)OEt | CH(OMe)SMe |
| CH₂C(O)OEt | CH(OMe)S(O)Me |
| CH₂C(O)OEt | CH(OMe)S(O)₂Me |
| CH₂C(O)OEt | CH(OMe)SEt |
| CH₂C(O)OEt | CH(OMe)S(O)Et |
| CH₂C(O)OEt | CH(OMe)S(O)₂Et |
| CH₂C(O)OEt | CH(OMe)S(═NCN)Et |
| CH₂C(O)OEt | CH(OMe)S(O)(═NCN)Et |
| CH₂C(O)OEt | CH(OEt)SMe |
| CH₂C(O)OEt | CH(OEt)S(O)Me |
| CH₂C(O)OEt | CH(OEt)S(O)₂Me |
| CH₂C(O)OEt | CH(OEt)SEt |
| CH₂C(O)OEt | CH(OEt)S(O)Et |
| CH₂C(O)OEt | CH(OEt)S(O)₂Et |
| CH₂C(O)OEt | CH(OEt)S(═NCN)Et |
| CH₂C(O)OEt | CH(OEt)S(O)(═NCN)Et |
| CH₂C(O)OEt | CH(SMe)₂ |
| CH₂C(O)OEt | CH(SMe)S(O)Me |
| CH₂C(O)OEt | CH(SMe)S(O)₂Me |
| CH₂C(O)OEt | CH(OMe)SEt |
| CH₂C(O)OEt | CH(SMe)S(O)Et |
| CH₂C(O)OEt | CH(SMe)S(O)₂Et |
| CH₂C(O)OEt | CH(SMe)S(═NCN)Et |
| CH₂C(O)OEt | CH(SMe)S(O)(═NCN)Et |
| CH₂C(O)OEt | CH(SEt)SMe |
| CH₂C(O)OEt | CH(SEt)S(O)Me |
| CH₂C(O)OEt | CH(SEt)S(O)₂Me |
| CH₂C(O)OEt | CH(SEt)SEt |
| CH₂C(O)OEt | CH(SEt)S(O)Et |
| CH₂C(O)OEt | CH(SEt)S(O)₂Et |
| CH₂C(O)OEt | CH(SEt)S(═NCN)Et |
| CH₂C(O)OEt | CH(SEt)S(O)(═NCN)Et |
| CH₂C(O)OEt | CH(CN)SMe |
| CH₂C(O)OEt | CH(CN)S(O)Me |
| CH₂C(O)OEt | CH(CN)S(O)₂Me |
| CH₂C(O)OEt | CH(CN)SEt |
| CH₂C(O)OEt | CH(CN)S(O)Et |
| CH₂C(O)OEt | CH(CN)S(O)₂Et |
| CH₂C(O)OEt | CH(CN)S(═NCN)Et |
| CH₂C(O)OEt | CH(CN)S(O)(═NCN)Et |
| CH₂C(O)OEt | CH{C(O)OMe}SMe |
| CH₂C(O)OEt | CH{C(O)OMe}S(O)Me |
| CH₂C(O)OEt | CH{C(O)OMe}S(O)₂Me |
| CH₂C(O)OEt | CH{C(O)OMe}SMe |
| CH₂C(O)OEt | CH{C(O)OMe}S(O)Me |
| CH₂C(O)OEt | CH{C(O)OMe}S(O)₂Me |
| CH₂C(O)OEt | CH{C(O)OMe}SEt |
| CH₂C(O)OEt | CH{C(O)OMe}S(O)Et |
| CH₂C(O)OEt | CH{C(O)OMe}S(O)₂Et |
| CH₂C(O)OEt | CH{C(O)OMe}S(═NCN)Et |
| CH₂C(O)OEt | CH{C(O)OMe}S(O)(═NCN)Et |
| CH₂C(O)OEt | CH{C(O)OEt}SMe |
| CH₂C(O)OEt | CH{C(O)OEt}S(O)Me |
| CH₂C(O)OEt | CH{C(O)OEt}S(O)₂Me |
| CH₂C(O)OEt | CH{C(O)OEt}SEt |
| CH₂C(O)OEt | CH{C(O)OEt}S(O)Et |
| CH₂C(O)OEt | CH{C(O)OEt}S(O)₂Et |
| CH₂C(O)OEt | CH{C(O)OEt}S(═NCN)Et |
| CH₂C(O)OEt | CH{C(O)OEt}S(O)(═NCN)Et |
| CH₂C(O)OEt | C(Me)₂SMe |
| CH₂C(O)OEt | C(Me)₂S(O)Me |
| CH₂C(O)OEt | C(Me)₂S(O)₂Me |
| CH₂C(O)OEt | CH₂CH₂SMe |
| CH₂C(O)OEt | CH₂CH₂S(O)Me |
| CH₂C(O)OEt | CH₂CH₂S(O)₂Me |
| CH₂C(O)OEt | CH(Me)CH₂SMe |
| CH₂C(O)OEt | CH(Me)CH₂S(O)Me |
| CH₂C(O)OEt | CH(Me)CH₂S(O)₂Me |
| C(O)OMe | CH₂SMe |
| C(O)OMe | CH₂S(O)Me |
| C(O)OMe | CH₂S(O)₂Me |
| C(O)OMe | CH₂SEt |
| C(O)OMe | CH₂S(O)Et |
| C(O)OMe | CH₂S(O)₂Et |
| C(O)OMe | CH₂SPr-n |
| C(O)OMe | CH₂S(O)Pr-n |
| C(O)OMe | CH₂S(O)₂Pr-n |
| C(O)OMe | CH₂SPr-i |
| C(O)OMe | CH₂S(O)Pr-i |
| C(O)OMe | CH₂S(O)₂Pr-i |
| C(O)OMe | CH₂SPr-c |
| C(O)OMe | CH₂S(O)Pr-c |
| C(O)OMe | CH₂S(O)₂Pr-c |
| C(O)OMe | CH₂SCH₂CH═CH₂ |
| C(O)OMe | CH₂S(O)CH₂CH═CH₂ |
| C(O)OMe | CH₂S(O)₂CH₂CH═CH₂ |
| C(O)OMe | CH₂SCH₂C≡CH |
| C(O)OMe | CH₂S(O)CH₂C≡CH |
| C(O)OMe | CH₂S(O)₂CH₂C≡CH |
| C(O)OMe | CH₂SCH₂CF₃ |
| C(O)OMe | CH₂S(O)CH₂CF₃ |
| C(O)OMe | CH₂S(O)₂CH₂CF₃ |
| C(O)OMe | CH₂SCH₂CH(OMe)₂ |
| C(O)OMe | CH₂SCH₂CH(═NOMe) |
| C(O)OMe | CH(Me)SMe |
| C(O)OMe | CH(Me)S(O)Me |
| C(O)OMe | CH(Me)S(O)₂Me |
| C(O)OMe | CH(Me)SEt |
| C(O)OMe | CH(Me)S(O)Et |
| C(O)OMe | CH(Me)S(O)₂Et |
| C(O)OMe | CH(Me)S(═NCN)Et |
| C(O)OMe | CH(Me)S(O)(═NCN)Et |
| C(O)OMe | CH(Me)SPr-n |
| C(O)OMe | CH(Me)S(O)Pr-n |
| C(O)OMe | CH(Me)S(O)₂Pr-n |
| C(O)OMe | CH(Me)SPr-i |
| C(O)OMe | CH(Me)S(O)Pr-i |
| C(O)OMe | CH(Me)S(O)₂Pr-i |
| C(O)OMe | CH(Me)SBu-t |
| C(O)OMe | CH(Me)S(O)Bu-t |
| C(O)OMe | CH(Me)S(O)₂Bu-t |
| C(O)OMe | CH(Me)SCH₂Pr-c |
| C(O)OMe | CH(Me)S(O)CH₂Pr-c |
| C(O)OMe | CH(Me)S(O)₂CH₂Pr-c |
| C(O)OMe | CH(Me)SCH₂OMe |
| C(O)OMe | CH(Me)SCH₂SMe |
| C(O)OMe | CH(Me)S(O)CH₂SMe |
| C(O)OMe | CH(Me)S(O)₂CH₂SMe |
| C(O)OMe | CH(Me)SCH₂C═CH₂ |
| C(O)OMe | CH(Me)S(O)CH₂C═CH₂ |
| C(O)OMe | CH(Me)S(O)₂CH₂C═CH₂ |
| C(O)OMe | CH(Me)SCH₂C≡CH |
| C(O)OMe | CH(Me)S(O)CH₂C≡CH |
| C(O)OMe | CH(Me)S(O)₂CH₂C≡CH |
| C(O)OMe | CH(Me)SCH₂C(O)NHMe |
| C(O)OMe | CH(Me)S(O)CH₂C(O)NHMe |
| C(O)OMe | CH(Me)S(O)₂CH₂C(O)NHMe |
| C(O)OMe | CH(Me)SCH₂C(O)OMe |
| C(O)OMe | CH(Me)S(O)CH₂C(O)OMe |
| C(O)OMe | CH(Me)S(O)₂CH₂C(O)OMe |
| C(O)OMe | CH(Me)SCH₂CH₂CH₂Cl |
| C(O)OMe | CH(Me)S(O)CH₂CH₂CH₂Cl |
| C(O)OMe | CH(Me)S(O)₂CH₂CH₂CH₂Cl |
| C(O)OMe | CH(Me)SCH₂CF₃ |
| C(O)OMe | CH(Me)S(O)CH₂CF₃ |
| C(O)OMe | CH(Me)S(O)₂CH₂CF₃ |
| C(O)OMe | CH(Me)SCH₂Ph |
| C(O)OMe | CH(Me)S(O)CH₂Ph |
| C(O)OMe | CH(Me)S(O)₂CH₂Ph |
| C(O)OMe | CH(Me)SCH₂(D1-34a) |
| C(O)OMe | CH(Me)S(O)CH₂(D1-34a) |
| C(O)OMe | CH(Me)S(O)₂CH₂(D1-34a) |
| C(O)OMe | CH(CH₃)SCH₂Si(CH₃)₃ |
| C(O)OMe | CH(Me)SCN |
| C(O)OMe | CH(Me)SCH₂CN |
| C(O)OMe | CH(Me)SC(O)Me |
| C(O)OMe | CH(Me)S{D1-12a(X^{1a}═Me)} |
| C(O)OMe | CH(Me)S(D1-32a) |
| C(O)OMe | CH(Me)S{D1-32b(3-NO₂)} |
| C(O)OMe | CH(Me)S{D1-32b(3-CF₃)} |
| C(O)OMe | CH(Me)S(D1-37a) |
| C(O)OMe | CH(Me)S(D1-51a) |
| C(O)OMe | CH(Et)SMe |
| C(O)OMe | CH(Et)S(O)Me |
| C(O)OMe | CH(Et)S(O)₂Me |
| C(O)OMe | CH(Et)SEt |
| C(O)OMe | CH(Et)S(O)Et |
| C(O)OMe | CH(Et)S(O)₂Et |
| C(O)OMe | CH(Et)SCH₂CF₃ |
| C(O)OMe | CH(Et)S(O)CH₂CF₃ |
| C(O)OMe | CH(Et)S(O)₂CH₂CF₃ |
| C(O)OMe | CH(Pr-n)SMe |
| C(O)OMe | CH(Pr-n)S(O)Me |
| C(O)OMe | CH(Pr-n)S(O)₂Me |
| C(O)OMe | CH(Pr-n)SEt |
| C(O)OMe | CH(Pr-n)S(O)Et |
| C(O)OMe | CH(Pr-n)S(O)₂Et |
| C(O)OMe | CH(Pr-n)S(═NCN)Et |
| C(O)OMe | CH(Pr-n)S(O)(═NCN)Et |
| C(O)OMe | CH(Pr-n)SPr-n |
| C(O)OMe | CH(Pr-n)S(O)Pr-n |
| C(O)OMe | CH(Pr-n)S(O)₂Pr-n |
| C(O)OMe | CH(Pr-n)SPr-i |
| C(O)OMe | CH(Pr-n)S(O)Pr-i |
| C(O)OMe | CH(Pr-n)S(O)₂Pr-i |
| C(O)OMe | CH(Pr-n)SPr-c |
| C(O)OMe | CH(Pr-n)S(O)Pr-c |
| C(O)OMe | CH(Pr-n)S(O)₂Pr-c |
| C(O)OMe | CH(Pr-n)SCH₂CF₃ |
| C(O)OMe | CH(Pr-n)S(O)CH₂CF₃ |
| C(O)OMe | CH(Pr-n)S(O)₂CH₂CF₃ |
| C(O)OMe | CH(Pr-i)SMe |
| C(O)OMe | CH(Pr-i)S(O)Me |
| C(O)OMe | CH(Pr-i)S(O)₂Me |
| C(O)OMe | CH(Pr-i)SEt |
| C(O)OMe | CH(Pr-i)S(O)Et |
| C(O)OMe | CH(Pr-i)S(O)₂Et |
| C(O)OMe | CH(Pr-i)S(═NCN)Et |
| C(O)OMe | CH(Pr-i)S(O)(═NCN)Et |
| C(O)OMe | CH(Pr-i)SPr-n |
| C(O)OMe | CH(Pr-i)S(O)Pr-n |
| C(O)OMe | CH(Pr-i)S(O)₂Pr-n |
| C(O)OMe | CH(Pr-i)SPr-i |
| C(O)OMe | CH(Pr-i)S(O)Pr-i |
| C(O)OMe | CH(Pr-i)S(O)₂Pr-i |
| C(O)OMe | CH(Pr-i)SPr-c |
| C(O)OMe | CH(Pr-i)S(O)Pr-c |
| C(O)OMe | CH(Pr-i)S(O)₂Pr-c |
| C(O)OMe | CH(Pr-i)SCH₂CF₃ |
| C(O)OMe | CH(Pr-i)S(O)CH₂CF₃ |
| C(O)OMe | CH(Pr-i)S(O)₂CH₂CF₃ |
| C(O)OMe | CH(Pr-c)SMe |
| C(O)OMe | CH(Pr-c)S(O)Me |
| C(O)OMe | CH(Pr-c)S(O)₂Me |
| C(O)OMe | CH(Pr-c)SEt |
| C(O)OMe | CH(Pr-c)S(O)Et |
| C(O)OMe | CH(Pr-c)S(O)₂Et |
| C(O)OMe | CH(Pr-c)S(═NCN)Et |
| C(O)OMe | CH(Pr-c)S(O)(═NCN)Et |
| C(O)OMe | CH(Pr-c)SPr-n |
| C(O)OMe | CH(Pr-c)S(O)Pr-n |
| C(O)OMe | CH(Pr-c)S(O)₂Pr-n |
| C(O)OMe | CH(Pr-c)SPr-i |
| C(O)OMe | CH(Pr-c)S(O)Pr-i |
| C(O)OMe | CH(Pr-c)S(O)₂Pr-i |
| C(O)OMe | CH(Pr-c)SPr-c |
| C(O)OMe | CH(Pr-c)S(O)Pr-c |
| C(O)OMe | CH(Pr-c)S(O)₂Pr-c |
| C(O)OMe | CH(Pr-c)SCH₂CF₃ |
| C(O)OMe | CH(Pr-c)S(O)CH₂CF₃ |
| C(O)OMe | CH(Pr-c)S(O)₂CH₂CF₃ |
| C(O)OMe | CH(Bu-n)SMe |
| C(O)OMe | CH(Bu-n)S(O)Me |
| C(O)OMe | CH(Bu-n)S(O)₂Me |
| C(O)OMe | CH(Bu-n)SEt |
| C(O)OMe | CH(Bu-n)S(O)Et |
| C(O)OMe | CH(Bu-n)S(O)₂Et |
| C(O)OMe | CH(Bu-n)S(═NCN)Et |
| C(O)OMe | CH(Bu-n)S(O)(═NCN)Et |
| C(O)OMe | CH(Bu-n)SPr-n |
| C(O)OMe | CH(Bu-n)S(O)Pr-n |
| C(O)OMe | CH(Bu-n)S(O)₂Pr-n |
| C(O)OMe | CH(Bu-n)SPr-i |
| C(O)OMe | CH(Bu-n)S(O)Pr-i |
| C(O)OMe | CH(Bu-n)S(O)₂Pr-i |
| C(O)OMe | CH(Bu-n)SPr-c |
| C(O)OMe | CH(Bu-n)S(O)Pr-c |
| C(O)OMe | CH(Bu-n)S(O)₂Pr-c |
| C(O)OMe | CH(Bu-n)SCH₂CF₃ |
| C(O)OMe | CH(Bu-n)S(O)CH₂CF₃ |
| C(O)OMe | CH(Bu-n)S(O)₂CH₂CF₃ |
| C(O)OMe | CH(F)SMe |
| C(O)OMe | CH(F)S(O)Me |
| C(O)OMe | CH(F)S(O)₂Me |
| C(O)OMe | CH(F)SEt |
| C(O)OMe | CH(F)S(O)Et |
| C(O)OMe | CH(F)S(O)₂Et |
| C(O)OMe | CH(F)S(═NCN)Et |
| C(O)OMe | CH(F)S(O)(═NCN)Et |
| C(O)OMe | CH(F)SCH₂CF₃ |
| C(O)OMe | CH(F)S(O)CH₂CF₃ |
| C(O)OMe | CH(F)S(O)₂CH₂CF₃ |
| C(O)OMe | C(F)₂SMe |
| C(O)OMe | C(F)₂S(O)Me |
| C(O)OMe | C(F)₂S(O)₂Me |
| C(O)OMe | C(F)₂SEt |
| C(O)OMe | C(F)₂S(O)Et |
| C(O)OMe | C(F)₂S(O)₂Et |
| C(O)OMe | C(F)₂S(═NCN)Et |
| C(O)OMe | C(F)₂S(O)(═NCN)Et |
| C(O)OMe | C(F)₂SCH₂CF₃ |
| C(O)OMe | C(F)₂S(O)CH₂CF₃ |
| C(O)OMe | C(F)₂S(O)₂CH₂CF₃ |
| C(O)OMe | CH(Cl)SMe |
| C(O)OMe | CH(Cl)S(O)Me |
| C(O)OMe | CH(Cl)S(O)₂Me |
| C(O)OMe | CH(Cl)SEt |
| C(O)OMe | CH(Cl)S(O)Et |
| C(O)OMe | CH(Cl)S(O)₂Et |
| C(O)OMe | CH(Cl)S(═NCN)Et |
| C(O)OMe | CH(Cl)S(O)(═NCN)Et |
| C(O)OMe | CH(Cl)SCH₂CF₃ |
| C(O)OMe | CH(Cl)S(O)CH₂CF₃ |
| C(O)OMe | CH(Cl)S(O)₂CH₂CF₃ |
| C(O)OMe | C(Cl)₂SMe |
| C(O)OMe | C(Cl)₂S(O)Me |
| C(O)OMe | C(Cl)₂S(O)₂Me |
| C(O)OMe | C(Cl)₂SEt |
| C(O)OMe | C(Cl)₂S(O)Et |
| C(O)OMe | C(Cl)₂S(O)₂Et |
| C(O)OMe | C(Cl)2S(═NCN)Et |
| C(O)OMe | C(Cl)₂S(O)(═NCN)Et |
| C(O)OMe | C(Cl)₂SCH₂CF₃ |
| C(O)OMe | C(Cl)₂S(O)CH₂CF₃ |
| C(O)OMe | C(Cl)₂S(O)₂CH₂CF₃ |
| C(O)OMe | CH(Br)SMe |
| C(O)OMe | CH(Br)S(O)Me |
| C(O)OMe | CH(Br)S(O)₂Me |
| C(O)OMe | CH(Br)SEt |
| C(O)OMe | CH(Br)S(O)Et |
| C(O)OMe | CH(Br)S(O)₂Et |
| C(O)OMe | CH(Br)S(═NCN)Et |
| C(O)OMe | CH(Br)S(O)(═NCN)Et |
| C(O)OMe | CH(Br)SCH₂CF₃ |
| C(O)OMe | CH(Br)S(O)CH₂CF₃ |
| C(O)OMe | CH(Br)S(O)₂CH₂CF₃ |
| C(O)OMe | CH(I)SMe |
| C(O)OMe | CH(I)S(O)Me |
| C(O)OMe | CH(I)S(O)₂Me |
| C(O)OMe | CH(I)SEt |
| C(O)OMe | CH(I)S(O)Et |
| C(O)OMe | CH(I)S(O)₂Et |
| C(O)OMe | CH(I)S(═NCN)Et |
| C(O)OMe | CH(I)S(O)(═NCN)Et |
| C(O)OMe | CH(I)SCH₂CF₃ |
| C(O)OMe | CH(I)S(O)CH₂CF₃ |
| C(O)OMe | CH(I)S(O)₂CH₂CF₃ |
| C(O)OMe | CH{OC(O)Me}SMe |
| C(O)OMe | CH{OC(O)Me}S(O)Me |
| C(O)OMe | CH{OC(O)Me}S(O)₂Me |
| C(O)OMe | CH{OC(O)OMe}SEt |
| C(O)OMe | CH{OC(O)OMe}S(O)Et |
| C(O)OMe | CH{OC(O)OMe}S(O)₂Et |
| C(O)OMe | CH{OC(O)OMe}S(═NCN)Et |
| C(O)OMe | CH{OC(O)OMe}S(O)(═NCN)Et |
| C(O)OMe | CH{OC(O)OEt}SMe |
| C(O)OMe | CH{OC(O)OEt}S(O)Me |
| C(O)OMe | CH{OC(O)OEt}S(O)₂Me |
| C(O)OMe | CH{OC(O)OEt}SEt |
| C(O)OMe | CH{OC(O)OEt}S(O)Et |
| C(O)OMe | CH{OC(O)OEt}S(O)₂Et |
| C(O)OMe | CH{OC(O)OEt}S(═NCN)Et |
| C(O)OMe | CH{OC(O)OEt}S(O)(═NCN)Et |
| C(O)OMe | CH(OMe)SMe |
| C(O)OMe | CH(OMe)S(O)Me |
| C(O)OMe | CH(OMe)S(O)₂Me |
| C(O)OMe | CH(OMe)SEt |
| C(O)OMe | CH(OMe)S(O)Et |
| C(O)OMe | CH(OMe)S(O)₂Et |
| C(O)OMe | CH(OMe)S(═NCN)Et |
| C(O)OMe | CH(OMe)S(O)(═NCN)Et |
| C(O)OMe | CH(OEt)SMe |
| C(O)OMe | CH(OEt)S(O)Me |
| C(O)OMe | CH(OEt)S(O)₂Me |
| C(O)OMe | CH(OEt)SEt |
| C(O)OMe | CH(OEt)S(O)Et |
| C(O)OMe | CH(OEt)S(O)₂Et |
| C(O)OMe | CH(OEt)S(═NCN)Et |
| C(O)OMe | CH(OEt)S(O)(═NCN)Et |
| C(O)OMe | CH(SMe)₂ |
| C(O)OMe | CH(SMe)S(O)Me |
| C(O)OMe | CH(SMe)S(O)₂Me |
| C(O)OMe | CH(SMe)SEt |
| C(O)OMe | CH(SMe)S(O)Et |
| C(O)OMe | CH(SMe)S(O)₂Et |
| C(O)OMe | CH(SMe)S(═NCN)Et |
| C(O)OMe | CH(SMe)S(O)(═NCN)Et |
| C(O)OMe | CH(SEt)SMe |
| C(O)OMe | CH(SEt)S(O)Me |
| C(O)OMe | CH(SEt)S(O)₂Me |
| C(O)OMe | CH(SEt)SEt |
| C(O)OMe | CH(SEt)S(O)Et |
| C(O)OMe | CH(SEt)S(O)₂Et |
| C(O)OMe | CH(SEt)S(═NCN)Et |
| C(O)OMe | CH(SEt)S(O)(═NCN)Et |
| C(O)OMe | CH(CN)SMe |
| C(O)OMe | CH(CN)S(O)Me |
| C(O)OMe | CH(CN)S(O)₂Me |
| C(O)OMe | CH(CN)SEt |
| C(O)OMe | CH(CN)S(O)Et |
| C(O)OMe | CH(CN)S(O)₂Et |
| C(O)OMe | CH(CN)S(═NCN)Et |
| C(O)OMe | CH(CN)S(O)(═NCN)Et |
| C(O)OMe | CH{C(O)OMe}SMe |
| C(O)OMe | CH{C(O)OMe}S(O)Me |
| C(O)OMe | CH{C(O)OMe}S(O)₂Me |
| C(O)OMe | CH{C(O)OMe}SMe |
| C(O)OMe | CH{C(O)OMe}S(O)Me |
| C(O)OMe | CH{C(O)OMe}S(O)₂Me |
| C(O)OMe | CH{C(O)OMe}SEt |
| C(O)OMe | CH{C(O)OMe}S(O)Et |
| C(O)OMe | CH{C(O)OMe}S(O)₂Et |
| C(O)OMe | CH{C(O)OMe}S(═NCN)Et |
| C(O)OMe | CH{C(O)OMe}S(O)(═NCN)Et |
| C(O)OMe | CH{C(O)OEt}SMe |
| C(O)OMe | CH{C(O)OEt}S(O)Me |
| C(O)OMe | CH{C(O)OEt}S(O)₂Me |
| C(O)OMe | CH{C(O)OEt}SEt |
| C(O)OMe | CH{C(O)OEt}S(O)Et |
| C(O)OMe | CH{C(O)OEt}S(O)₂Et |
| C(O)OMe | CH{C(O)OEt}S(═NCN)Et |
| C(O)OMe | CH{C(O)OEt}S(O)(═NCN)Et |
| C(O)OMe | C(Me)₂SMe |
| C(O)OMe | C(Me)₂S(O)Me |
| C(O)OMe | C(Me)₂S(O)₂Me |
| C(O)OMe | CH₂CH₂SMe |
| C(O)OMe | CH₂CH₂S(O)Me |
| C(O)OMe | CH₂CH₂S(O)₂Me |
| C(O)OMe | CH(Me)CH₂SMe |
| C(O)OMe | CH(Me)CH₂S(O)Me |
| C(O)OMe | CH(Me)CH₂S(O)₂Me |
| C(O)OEt | CH₂SMe |
| C(O)OEt | CH₂S(O)Me |
| C(O)OEt | CH₂S(O)₂Me |
| C(O)OEt | CH₂SEt |
| C(O)OEt | CH₂S(O)Et |
| C(O)OEt | CH₂S(O)₂Et |
| C(O)OEt | CH₂SPr-n |
| C(O)OEt | CH₂S(O)Pr-n |
| C(O)OEt | CH₂S(O)₂Pr-n |
| C(O)OEt | CH₂SPr-i |
| C(O)OEt | CH₂S(O)Pr-i |
| C(O)OEt | CH₂S(O)₂Pr-i |
| C(O)OEt | CH₂SPr-c |
| C(O)OEt | CH₂S(O)Pr-c |
| C(O)OEt | CH₂S(O)₂Pr-c |
| C(O)OEt | CH₂SCH₂CH═CH₂ |
| C(O)OEt | CH₂S(O)CH₂CH═CH2 |
| C(O)OEt | CH₂S(O)₂CH₂CH═CH₂ |
| C(O)OEt | CH₂SCH₂C≡CH |
| C(O)OEt | CH₂S(O)CH₂C≡CH |
| C(O)OEt | CH₂S(O)₂CH₂C≡CH |
| C(O)OEt | CH₂SCH₂CF₃ |
| C(O)OEt | CH₂S(O)CH₂CF₃ |
| C(O)OEt | CH₂S(O)₂CH₂CF₃ |
| C(O)OEt | CH₂SCH₂CH(OMe)₂ |
| C(O)OEt | CH₂SCH₂CH(═NOMe) |
| C(O)OEt | CH(Me)SMe |
| C(O)OEt | CH(Me)S(O)Me |
| C(O)OEt | CH(Me)S(O)₂Me |
| C(O)OEt | CH(Me)SEt |
| C(O)OEt | CH(Me)S(O)Et |
| C(O)OEt | CH(Me)S(O)₂Et |
| C(O)OEt | CH(Me)S(═NCN)Et |
| C(O)OEt | CH(Me)S(O)(═NCN)Et |
| C(O)OEt | CH(Me)SPr-n |
| C(O)OEt | CH(Me)S(O)Pr-n |
| C(O)OEt | CH(Me)S(O)₂Pr-n |
| C(O)OEt | CH(Me)SPr-i |
| C(O)OEt | CH(Me)S(O)Pr-i |
| C(O)OEt | CH(Me)S(O)₂Pr-i |
| C(O)OEt | CH(Me)SBu-t |
| C(O)OEt | CH(Me)S(O)Bu-t |
| C(O)OEt | CH(Me)S(O)₂Bu-t |
| C(O)OEt | CH(Me)SCH₂Pr-c |
| C(O)OEt | CH(Me)S(O)CH₂Pr-c |
| C(O)OEt | CH(Me)S(O)₂CH₂Pr-c |
| C(O)OEt | CH(Me)SCH₂OMe |
| C(O)OEt | CH(Me)SCH₂SMe |
| C(O)OEt | CH(Me)S(O)CH₂SMe |
| C(O)OEt | CH(Me)S(O)₂CH₂SMe |
| C(O)OEt | CH(Me)SCH₂C═CH₂ |
| C(O)OEt | CH(Me)S(O)CH₂C═CH₂ |
| C(O)OEt | CH(Me)S(O)₂CH₂C═CH₂ |
| C(O)OEt | CH(Me)SCH₂C≡CH |
| C(O)OEt | CH(Me)S(O)CH₂C≡CH |
| C(O)OEt | CH(Me)S(O)₂CH₂C≡CH |
| C(O)OEt | CH(Me)SCH₂C(O)NHMe |
| C(O)OEt | CH(Me)S(O)CH₂C(O)NHMe |
| C(O)OEt | CH(Me)S(O)₂CH₂C(O)NHMe |
| C(O)OEt | CH(Me)SCH₂C(O)OMe |
| C(O)OEt | CH(Me)S(O)CH₂C(O)OMe |
| C(O)OEt | CH(Me)S(O)₂CH₂C(O)OMe |
| C(O)OEt | CH(Me)SCH₂CH₂CH₂Cl |
| C(O)OEt | CH(Me)S(O)CH₂CH₂CH₂Cl |
| C(O)OEt | CH(Me)S(O)₂CH₂CH₂CH₂Cl |
| C(O)OEt | CH(Me)SCH₂CF₃ |
| C(O)OEt | CH(Me)S(O)CH₂CF₃ |
| C(O)OEt | CH(Me)S(O)₂CH₂CF₃ |
| C(O)OEt | CH(Me)SCH₂Ph |
| C(O)OEt | CH(Me)S(O)CH₂Ph |
| C(O)OEt | CH(Me)S(O)₂CH₂Ph |
| C(O)OEt | CH(Me)SCH₂(D1-34a) |
| C(O)OEt | CH(Me)S(O)CH₂(D1-34a) |
| C(O)OEt | CH(Me)S(O)₂CH₂(D1-34a) |
| C(O)OEt | CH(CH₃)SCH₂Si(CH₃)₃ |
| C(O)OEt | CH(Me)SCN |
| C(O)OEt | CH(Me)SCH₂CN |
| C(O)OEt | CH(Me)SC(O)Me |
| C(O)OEt | CH(Me)S{D1-12a(X^{1a}═Me)} |
| C(O)OEt | CH(Me)S(D1-32a) |
| C(O)OEt | CH(Me)S{D1-32b(3-NO₂)} |
| C(O)OEt | CH(Me)S{D1-32b(3-CF₃)} |
| C(O)OEt | CH(Me)S(D1-37a) |
| C(O)OEt | CH(Me)S(D1-51a) |
| C(O)OEt | CH(Et)SMe |
| C(O)OEt | CH(Et)S(O)Me |
| C(O)OEt | CH(Et)S(O)₂Me |
| C(O)OEt | CH(Et)SEt |
| C(O)OEt | CH(Et)S(O)Et |
| C(O)OEt | CH(Et)S(O)₂Et |
| C(O)OEt | CH(Et)SCH₂CF₃ |
| C(O)OEt | CH(Et)S(O)CH₂CF₃ |
| C(O)OEt | CH(Et)S(O)₂CH₂CF₃ |
| C(O)OEt | CH(Pr-n)SMe |
| C(O)OEt | CH(Pr-n)S(O)Me |
| C(O)OEt | CH(Pr-n)S(O)₂Me |
| C(O)OEt | CH(Pr-n)SEt |
| C(O)OEt | CH(Pr-n)S(O)Et |
| C(O)OEt | CH(Pr-n)S(O)₂Et |
| C(O)OEt | CH(Pr-n)S(═NCN)Et |
| C(O)OEt | CH(Pr-n)S(O)(═NCN)Et |
| C(O)OEt | CH(Pr-n)SPr-n |
| C(O)OEt | CH(Pr-n)S(O)Pr-n |
| C(O)OEt | CH(Pr-n)S(O)₂Pr-n |
| C(O)OEt | CH(Pr-n)SPr-i |
| C(O)OEt | CH(Pr-n)S(O)Pr-i |
| C(O)OEt | CH(Pr-n)S(O)₂Pr-i |
| C(O)OEt | CH(Pr-n)SPr-c |
| C(O)OEt | CH(Pr-n)S(O)Pr-c |
| C(O)OEt | CH(Pr-n)S(O)₂Pr-c |
| C(O)OEt | CH(Pr-n)SCH₂CF₃ |
| C(O)OEt | CH(Pr-n)S(O)CH₂CF₃ |
| C(O)OEt | CH(Pr-n)S(O)₂CH₂CF₃ |
| C(O)OEt | CH(Pr-i)SMe |
| C(O)OEt | CH(Pr-i)S(O)Me |
| C(O)OEt | CH(Pr-i)S(O)₂Me |
| C(O)OEt | CH(Pr-i)SEt |
| C(O)OEt | CH(Pr-i)S(O)Et |
| C(O)OEt | CH(Pr-i)S(O)₂Et |
| C(O)OEt | CH(Pr-i)S(═NCN)Et |
| C(O)OEt | CH(Pr-i)S(O)(═NCN)Et |
| C(O)OEt | CH(Pr-i)SPr-n |
| C(O)OEt | CH(Pr-i)S(O)Pr-n |
| C(O)OEt | CH(Pr-i)S(O)₂Pr-n |
| C(O)OEt | CH(Pr-i)SPr-i |
| C(O)OEt | CH(Pr-i)S(O)Pr-i |
| C(O)OEt | CH(Pr-i)S(O)₂Pr-i |
| C(O)OEt | CH(Pr-i)SPr-c |
| C(O)OEt | CH(Pr-i)S(O)Pr-c |
| C(O)OEt | CH(Pr-i)S(O)₂Pr-c |
| C(O)OEt | CH(Pr-i)SCH₂CF₃ |
| C(O)OEt | CH(Pr-i)S(O)CH₂CF₃ |
| C(O)OEt | CH(Pr-i)S(O)₂CH₂CF₃ |
| C(O)OEt | CH(Pr-c)SMe |
| C(O)OEt | CH(Pr-c)S(O)Me |
| C(O)OEt | CH(Pr-c)S(O)₂Me |
| C(O)OEt | CH(Pr-c)SEt |
| C(O)OEt | CH(Pr-c)S(O)Et |
| C(O)OEt | CH(Pr-c)S(O)₂Et |
| C(O)OEt | CH(Pr-c)S(═NCN)Et |
| C(O)OEt | CH(Pr-c)S(O)(═NCN)Et |
| C(O)OEt | CH(Pr-c)SPr-n |
| C(O)OEt | CH(Pr-c)S(O)Pr-n |
| C(O)OEt | CH(Pr-c)S(O)₂Pr-n |
| C(O)OEt | CH(Pr-c)SPr-i |
| C(O)OEt | CH(Pr-c)S(O)Pr-i |
| C(O)OEt | CH(Pr-c)S(O)₂Pr-i |
| C(O)OEt | CH(Pr-c)SPr-c |
| C(O)OEt | CH(Pr-c)S(O)Pr-c |
| C(O)OEt | CH(Pr-c)S(O)₂Pr-c |
| C(O)OEt | CH(Pr-c)SCH₂CF₃ |
| C(O)OEt | CH(Pr-c)S(O)CH₂CF₃ |
| C(O)OEt | CH(Pr-c)S(O)₂CH₂CF₃ |
| C(O)OEt | CH(Bu-n)SMe |
| C(O)OEt | CH(Bu-n)S(O)Me |
| C(O)OEt | CH(Bu-n)S(O)₂Me |
| C(O)OEt | CH(Bu-n)SEt |
| C(O)OEt | CH(Bu-n)S(O)Et |
| C(O)OEt | CH(Bu-n)S(O)₂Et |
| C(O)OEt | CH(Bu-n)S(═NCN)Et |
| C(O)OEt | CH(Bu-n)S(O)(═NCN)Et |
| C(O)OEt | CH(Bu-n)SPr-n |
| C(O)OEt | CH(Bu-n)S(O)Pr-n |
| C(O)OEt | CH(Bu-n)S(O)₂Pr-n |
| C(O)OEt | CH(Bu-n)SPr-i |
| C(O)OEt | CH(Bu-n)S(O)Pr-i |
| C(O)OEt | CH(Bu-n)S(O)₂Pr-i |
| C(O)OEt | CH(Bu-n)SPr-c |
| C(O)OEt | CH(Bu-n)S(O)Pr-c |
| C(O)OEt | CH(Bu-n)S(O)₂Pr-c |
| C(O)OEt | CH(Bu-n)SCH₂CF₃ |
| C(O)OEt | CH(Bu-n)S(O)CH₂CF₃ |
| C(O)OEt | CH(Bu-n)S(O)₂CH₂CF₃ |
| C(O)OEt | CH(F)SMe |
| C(O)OEt | CH(F)S(O)Me |
| C(O)OEt | CH(F)S(O)₂Me |
| C(O)OEt | CH(F)SEt |
| C(O)OEt | CH(F)S(O)Et |
| C(O)OEt | CH(F)S(O)₂Et |
| C(O)OEt | CH(F)S(═NCN)Et |
| C(O)OEt | CH(F)S(O)(═NCN)Et |
| C(O)OEt | CH(F)SCH₂CF₃ |
| C(O)OEt | CH(F)S(O)CH₂CF₃ |
| C(O)OEt | CH(F)S(O)₂CH₂CF₃ |
| C(O)OEt | C(F)₂SMe |
| C(O)OEt | C(F)₂S(O)Me |
| C(O)OEt | C(F)₂S(O)₂Me |
| C(O)OEt | C(F)₂SEt |
| C(O)OEt | C(F)₂S(O)Et |
| C(O)OEt | C(F)₂S(O)₂Et |
| C(O)OEt | C(F)₂S(═NCN)Et |
| C(O)OEt | C(F)₂S(O)(═NCN)Et |
| C(O)OEt | C(F)₂SCH₂CF₃ |
| C(O)OEt | C(F)₂S(O)CH₂CF₃ |
| C(O)OEt | C(F)₂S(O)₂CH₂CF₃ |
| C(O)OEt | CH(Cl)SMe |
| C(O)OEt | CH(Cl)S(O)Me |
| C(O)OEt | CH(Cl)S(O)₂Me |
| C(O)OEt | CH(Cl)SEt |
| C(O)OEt | CH(Cl)S(O)Et |
| C(O)OEt | CH(Cl)S(O)₂Et |
| C(O)OEt | CH(Cl)S(═NCN)Et |
| C(O)OEt | CH(Cl)S(O)(═NCN)Et |
| C(O)OEt | CH(Cl)SCH₂CF₃ |
| C(O)OEt | CH(Cl)S(O)CH₂CF₃ |
| C(O)OEt | CH(Cl)S(O)₂CH₂CF₃ |
| C(O)OEt | C(Cl)₂SMe |
| C(O)OEt | C(Cl)₂S(O)Me |
| C(O)OEt | C(Cl)₂S(O)₂Me |
| C(O)OEt | C(Cl)₂SEt |
| C(O)OEt | C(Cl)₂S(O)Et |
| C(O)OEt | C(Cl)₂S(O)₂Et |
| C(O)OEt | C(Cl)₂S(═NCN)Et |
| C(O)OEt | C(Cl)₂S(O)(═NCN)Et |
| C(O)OEt | C(Cl)₂SCH₂CF₃ |
| C(O)OEt | C(Cl)₂S(O)CH₂CF₃ |
| C(O)OEt | C(Cl)₂S(O)₂CH₂CF₃ |
| C(O)OEt | CH(Br)SMe |
| C(O)OEt | CH(Br)S(O)Me |
| C(O)OEt | CH(Br)S(O)₂Me |
| C(O)OEt | CH(Br)SEt |
| C(O)OEt | CH(Br)S(O)Et |
| C(O)OEt | CH(Br)S(O)₂Et |
| C(O)OEt | CH(Br)S(═NCN)Et |
| C(O)OEt | CH(Br)S(O)(═NCN)Et |
| C(O)OEt | CH(Br)SCH₂CF₃ |
| C(O)OEt | CH(Br)S(O)CH₂CF₃ |
| C(O)OEt | CH(Br)S(O)₂CH₂CF₃ |
| C(O)OEt | CH(I)SMe |
| C(O)OEt | CH(I)S(O)Me |
| C(O)OEt | CH(I)S(O)₂Me |
| C(O)OEt | CH(I)SEt |
| C(O)OEt | CH(I)S(O)Et |
| C(O)OEt | CH(I)S(O)₂Et |
| C(O)OEt | CH(I)S(═NCN)Et |
| C(O)OEt | CH(I)S(O)(═NCN)Et |
| C(O)OEt | CH(I)SCH₂CF₃ |
| C(O)OEt | CH(I)S(O)CH₂CF₃ |
| C(O)OEt | CH(I)S(O)₂CH₂CF₃ |
| C(O)OEt | CH{OC(O)Me}SMe |
| C(O)OEt | CH{OC(O)Me}S(O)Me |
| C(O)OEt | CH{OC(O)Me}S(O)₂Me |
| C(O)OEt | CH{OC(O)OMe}SEt |
| C(O)OEt | CH{OC(O)OMe}S(O)Et |
| C(O)OEt | CH{OC(O)OMe}S(O)₂Et |
| C(O)OEt | CH{OC(O)OMe}S(═NCN)Et |
| C(O)OEt | CH{OC(O)OMe}S(O)(═NCN)Et |
| C(O)OEt | CH{OC(O)OEt}SMe |
| C(O)OEt | CH{OC(O)OEt}S(O)Me |
| C(O)OEt | CH{OC(O)OEt}S(O)₂Me |
| C(O)OEt | CH{OC(O)OEt}SEt |
| C(O)OEt | CH{OC(O)OEt}S(O)Et |
| C(O)OEt | CH{OC(O)OEt}S(O)₂Et |
| C(O)OEt | CH{OC(O)OEt}S(═NCN)Et |
| C(O)OEt | CH{OC(O)OEt}S(O)(═NCN)Et |
| C(O)OEt | CH(OMe)SMe |
| C(O)OEt | CH(OMe)S(O)Me |
| C(O)OEt | CH(OMe)S(O)₂Me |
| C(O)OEt | CH(OMe)SEt |
| C(O)OEt | CH(OMe)S(O)Et |
| C(O)OEt | CH(OMe)S(O)₂Et |
| C(O)OEt | CH(OMe)S(═NCN)Et |
| C(O)OEt | CH(OMe)S(O)(═NCN)Et |
| C(O)OEt | CH(OEt)SMe |
| C(O)OEt | CH(OEt)S(O)Me |
| C(O)OEt | CH(OEt)S(O)₂Me |
| C(O)OEt | CH(OEt)SEt |
| C(O)OEt | CH(OEt)S(O)Et |
| C(O)OEt | CH(OEt)S(O)₂Et |
| C(O)OEt | CH(OEt)S(═NCN)Et |
| C(O)OEt | CH(OEt)S(O)(═NCN)Et |
| C(O)OEt | CH(SMe)₂ |
| C(O)OEt | CH(SMe)S(O)Me |
| C(O)OEt | CH(SMe)S(O)₂Me |
| C(O)OEt | CH(SMe)SEt |
| C(O)OEt | CH(SMe)S(O)Et |
| C(O)OEt | CH(SMe)S(O)₂Et |
| C(O)OEt | CH(SMe)S(═NCN)Et |
| C(O)OEt | CH(SMe)S(O)(═NCN)Et |
| C(O)OEt | CH(SEt)SMe |
| C(O)OEt | CH(SEt)S(O)Me |
| C(O)OEt | CH(SEt)S(O)₂Me |
| C(O)OEt | CH(SEt)SEt |
| C(O)OEt | CH(SEt)S(O)Et |
| C(O)OEt | CH(SEt)S(O)₂Et |
| C(O)OEt | CH(SEt)S(═NCN)Et |
| C(O)OEt | CH(SEt)S(O)(═NCN)Et |
| C(O)OEt | CH(CN)SMe |
| C(O)OEt | CH(CN)S(O)Me |
| C(O)OEt | CH(CN)S(O)₂Me |
| C(O)OEt | CH(CN)SEt |
| C(O)OEt | CH(CN)S(O)Et |
| C(O)OEt | CH(CN)S(O)₂Et |
| C(O)OEt | CH(CN)S(═NCN)Et |
| C(O)OEt | CH(CN)S(O)(═NCN)Et |
| C(O)OEt | CH{C(O)OMe}SMe |
| C(O)OEt | CH{C(O)OMe}S(O)Me |
| C(O)OEt | CH{C(O)OMe}S(O)₂Me |
| C(O)OEt | CH{C(O)OMe}SMe |
| C(O)OEt | CH{C(O)OMe}S(O)Me |
| C(O)OEt | CH{C(O)OMe}S(O)₂Me |
| C(O)OEt | CH{C(O)OMe}SEt |
| C(O)OEt | CH{C(O)OMe}S(O)Et |
| C(O)OEt | CH{C(O)OMe}S(O)₂Et |
| C(O)OEt | CH{C(O)OMe}S(═NCN)Et |
| C(O)OEt | CH{C(O)OMe}S(O)(═NCN)Et |
| C(O)OEt | CH{C(O)OEt}SMe |
| C(O)OEt | CH{C(O)OEt}S(O)Me |
| C(O)OEt | CH{C(O)OEt}S(O)₂Me |
| C(O)OEt | CH{C(O)OEt}SEt |
| C(O)OEt | CH{C(O)OEt}S(O)Et |
| C(O)OEt | CH{C(O)OEt}S(O)₂Et |
| C(O)OEt | CH{C(O)OEt}S(═NCN)Et |
| C(O)OEt | CH{C(O)OEt}S(O)(═NCN)Et |
| C(O)OEt | C(Me)₂SMe |
| C(O)OEt | C(Me)₂S(O)Me |
| C(O)OEt | C(Me)₂S(O)₂Me |
| C(O)OEt | CH₂CH₂SMe |
| C(O)OEt | CH₂CH₂S(O)Me |
| C(O)OEt | CH₂CH₂S(O)₂Me |
| C(O)OEt | CH(Me)CH₂SMe |
| C(O)OEt | CH(Me)CH₂S(O)Me |
| C(O)OEt | CH(Me)CH₂S(O)₂Me |
| CH₂CF₃ | CH₂SMe |
| CH₂CF₃ | CH₂S(O)Me |
| CH₂CF₃ | CH₂S(O)₂Me |
| CH₂CF₃ | CH₂SEt |
| CH₂CF₃ | CH₂S(O)Et |
| CH₂CF₃ | CH₂S(O)₂Et |
| CH₂CF₃ | CH2SPr-n |
| CH₂CF₃ | CH₂S(O)Pr-n |
| CH₂CF₃ | CH₂S(O)₂Pr-n |
| CH₂CF₃ | CH₂SPr-i |
| CH₂CF₃ | CH₂S(O)Pr-i |
| CH₂CF₃ | CH₂S(O)₂Pr-i |
| CH₂CF₃ | CH₂SPr-c |
| CH₂CF₃ | CH₂S(O)Pr-c |
| CH₂CF₃ | CH₂S(O)₂Pr-c |
| CH₂CF₃ | CH₂SCH₂CH═CH₂ |
| CH₂CF₃ | CH₂S(O)CH₂CH═CH₂ |
| CH₂CF₃ | CH₂S(O)₂CH₂CH═CH₂ |
| CH₂CF₃ | CH₂SCH₂C≡CH |
| CH₂CF₃ | CH₂S(O)CH₂C≡CH |
| CH₂CF₃ | CH₂S(O)₂CH₂C≡CH |
| CH₂CF₃ | CH₂SCH₂CF₃ |
| CH₂CF₃ | CH₂S(O)CH₂CF₃ |
| CH₂CF₃ | CH₂S(O)₂CH₂CF₃ |
| CH₂CF₃ | CH₂SCH₂CH(OMe)₂ |
| CH₂CF₃ | CH₂SCH₂CH(═NOMe) |
| CH₂CF₃ | CH(Me)SMe |
| CH₂CF₃ | CH(Me)S(O)Me |
| CH₂CF₃ | CH(Me)S(O)₂Me |
| CH₂CF₃ | CH(Me)SEt |
| CH₂CF₃ | CH(Me)S(O)Et |
| CH₂CF₃ | CH(Me)S(O)₂Et |
| CH₂CF₃ | CH(Me)S(═NCN)Et |
| CH₂CF₃ | CH(Me)S(O)(═NCN)Et |
| CH₂CF₃ | CH(Me)SPr-n |
| CH₂CF₃ | CH(Me)S(O)Pr-n |
| CH₂CF₃ | CH(Me)S(O)₂Pr-n |
| CH₂CF₃ | CH(Me)SPr-i |
| CH₂CF₃ | CH(Me)S(O)Pr-i |
| CH₂CF₃ | CH(Me)S(O)₂Pr-i |
| CH₂CF₃ | CH(Me)SBu-t |
| CH₂CF₃ | CH(Me)S(O)Bu-t |
| CH₂CF₃ | CH(Me)S(O)₂Bu-t |
| CH₂CF₃ | CH(Me)SCH₂Pr-c |
| CH₂CF₃ | CH(Me)S(O)CH₂Pr-c |
| CH₂CF₃ | CH(Me)S(O)₂CH₂Pr-c |
| CH₂CF₃ | CH(Me)SCH₂OMe |
| CH₂CF₃ | CH(Me)SCH₂SMe |
| CH₂CF₃ | CH(Me)S(O)CH₂SMe |
| CH₂CF₃ | CH(Me)S(O)₂CH₂SMe |
| CH₂CF₃ | CH(Me)SCH₂C═CH₂ |
| CH₂CF₃ | CH(Me)S(O)CH₂C═CH₂ |
| CH₂CF₃ | CH(Me)S(O)₂CH₂C═CH₂ |
| CH₂CF₃ | CH(Me)SCH₂C≡CH |
| CH₂CF₃ | CH(Me)S(O)CH₂C≡CH |
| CH₂CF₃ | CH(Me)S(O)₂CH₂C≡CH |
| CH₂CF₃ | CH(Me)SCH₂C(O)NHMe |
| CH₂CF₃ | CH(Me)S(O)CH₂C(O)NHMe |
| CH₂CF₃ | CH(Me)S(O)₂CH₂C(O)NHMe |
| CH₂CF₃ | CH(Me)SCH₂C(O)OMe |
| CH₂CF₃ | CH(Me)S(O)CH₂C(O)OMe |
| CH₂CF₃ | CH(Me)S(O)₂CH₂C(O)OMe |
| CH₂CF₃ | CH(Me)SCH₂CH₂CH₂Cl |
| CH₂CF₃ | CH(Me)S(O)CH₂CH₂CH₂Cl |
| CH₂CF₃ | CH(Me)S(O)₂CH₂CH₂CH₂Cl |
| CH₂CF₃ | CH(Me)SCH₂CF₃ |
| CH₂CF₃ | CH(Me)S(O)CH₂CF₃ |
| CH₂CF₃ | CH(Me)S(O)₂CH₂CF₃ |
| CH₂CF₃ | CH(Me)SCH₂Ph |
| CH₂CF₃ | CH(Me)S(O)CH₂Ph |
| CH₂CF₃ | CH(Me)S(O)₂CH₂Ph |
| CH₂CF₃ | CH(Me)SCH₂(D1-34a) |
| CH₂CF₃ | CH(Me)S(O)CH₂(D1-34a) |
| CH₂CF₃ | CH(Me)S(O)₂CH₂(D1-34a) |
| CH₂CF₃ | CH(CH₃)SCH₂Si(CH₃)₃ |
| CH₂CF₃ | CH(Me)SCN |
| CH₂CF₃ | CH(Me)SCH₂CN |
| CH₂CF₃ | CH(Me)SC(O)Me |
| CH₂CF₃ | CH(Me)S{D1-12a(X^{1a}═Me)} |
| CH₂CF₃ | CH(Me)S(D1-32a) |
| CH₂CF₃ | CH(Me)S{D1-32b(3-NO₂)} |
| CH₂CF₃ | CH(Me)S{D1-32b(3-CF₃)} |
| CH₂CF₃ | CH(Me)S(D1-37a) |
| CH₂CF₃ | CH(Me)S(D1-51a) |
| CH₂CF₃ | CH(Et)SMe |
| CH₂CF₃ | CH(Et)S(O)Me |
| CH₂CF₃ | CH(Et)S(O)₂Me |
| CH₂CF₃ | CH(Et)SEt |
| CH₂CF₃ | CH(Et)S(O)Et |
| CH₂CF₃ | CH(Et)S(O)₂Et |
| CH₂CF₃ | CH(Et)SCH₂CF₃ |
| CH₂CF₃ | CH(Et)S(O)CH₂CF₃ |
| CH₂CF₃ | CH(Et)S(O)₂CH₂CF₃ |
| CH₂CF₃ | CH(Pr-n)SMe |
| CH₂CF₃ | CH(Pr-n)S(O)Me |
| CH₂CF₃ | CH(Pr-n)S(O)₂Me |
| CH₂CF₃ | CH(Pr-n)SEt |
| CH₂CF₃ | CH(Pr-n)S(O)Et |
| CH₂CF₃ | CH(Pr-n)S(O)₂Et |
| CH₂CF₃ | CH(Pr-n)S(═NCN)Et |
| CH₂CF₃ | CH(Pr-n)S(O)(═NCN)Et |
| CH₂CF₃ | CH(Pr-n)SPr-n |
| CH₂CF₃ | CH(Pr-n)S(O)Pr-n |
| CH₂CF₃ | CH(Pr-n)S(O)₂Pr-n |
| CH₂CF₃ | CH(Pr-n)SPr-i |
| CH₂CF₃ | CH(Pr-n)S(O)Pr-i |
| CH₂CF₃ | CH(Pr-n)S(O)₂Pr-i |
| CH₂CF₃ | CH(Pr-n)SPr-c |
| CH₂CF₃ | CH(Pr-n)S(O)Pr-c |
| CH₂CF₃ | CH(Pr-n)S(O)₂Pr-c |
| CH₂CF₃ | CH(Pr-n)SCH₂CF₃ |
| CH₂CF₃ | CH(Pr-n)S(O)CH₂CF₃ |
| CH₂CF₃ | CH(Pr-n)S(O)₂CH₂CF₃ |
| CH₂CF₃ | CH(Pr-i)SMe |
| CH₂CF₃ | CH(Pr-i)S(O)Me |
| CH₂CF₃ | CH(Pr-i)S(O)₂Me |
| CH₂CF₃ | CH(Pr-i)SEt |
| CH₂CF₃ | CH(Pr-i)S(O)Et |
| CH₂CF₃ | CH(Pr-i)S(O)₂Et |
| CH₂CF₃ | CH(Pr-i)S(═NCN)Et |
| CH₂CF₃ | CH(Pr-i)S(O)(═NCN)Et |
| CH₂CF₃ | CH(Pr-i)SPr-n |
| CH₂CF₃ | CH(Pr-i)S(O)Pr-n |
| CH₂CF₃ | CH(Pr-i)S(O)₂Pr-n |
| CH₂CF₃ | CH(Pr-i)SPr-i |
| CH₂CF₃ | CH(Pr-i)S(O)Pr-i |
| CH₂CF₃ | CH(Pr-i)S(O)₂Pr-i |
| CH₂CF₃ | CH(Pr-i)SPr-c |
| CH₂CF₃ | CH(Pr-i)S(O)Pr-c |
| CH₂CF₃ | CH(Pr-i)S(O)₂Pr-c |
| CH₂CF₃ | CH(Pr-i)SCH₂CF₃ |
| CH₂CF₃ | CH(Pr-i)S(O)CH₂CF₃ |
| CH₂CF₃ | CH(Pr-i)S(O)₂CH₂CF₃ |
| CH₂CF₃ | CH(Pr-c)SMe |
| CH₂CF₃ | CH(Pr-c)S(O)Me |
| CH₂CF₃ | CH(Pr-c)S(O)₂Me |
| CH₂CF₃ | CH(Pr-c)SEt |
| CH₂CF₃ | CH(Pr-c)S(O)Et |
| CH₂CF₃ | CH(Pr-c)S(O)₂Et |
| CH₂CF₃ | CH(Pr-c)S(═NCN)Et |
| CH₂CF₃ | CH(Pr-c)S(O)(═NCN)Et |
| CH₂CF₃ | CH(Pr-c)SPr-n |
| CH₂CF₃ | CH(Pr-c)S(O)Pr-n |
| CH₂CF₃ | CH(Pr-c)S(O)₂Pr-n |
| CH₂CF₃ | CH(Pr-c)SPr-i |
| CH₂CF₃ | CH(Pr-c)S(O)Pr-i |
| CH₂CF₃ | CH(Pr-c)S(O)₂Pr-i |
| CH₂CF₃ | CH(Pr-c)SPr-c |
| CH₂CF₃ | CH(Pr-c)S(O)Pr-c |
| CH₂CF₃ | CH(Pr-c)S(O)₂Pr-c |
| CH₂CF₃ | CH(Pr-c)SCH₂CF₃ |
| CH₂CF₃ | CH(Pr-c)S(O)CH₂CF₃ |
| CH₂CF₃ | CH(Pr-c)S(O)₂CH₂CF₃ |
| CH₂CF₃ | CH(Bu-n)SMe |
| CH₂CF₃ | CH(Bu-n)S(O)Me |
| CH₂CF₃ | CH(Bu-n)S(O)₂Me |
| CH₂CF₃ | CH(Bu-n)SEt |
| CH₂CF₃ | CH(Bu-n)S(O)Et |
| CH₂CF₃ | CH(Bu-n)S(O)₂Et |
| CH₂CF₃ | CH(Bu-n)S(═NCN)Et |
| CH₂CF₃ | CH(Bu-n)S(O)(═NCN)Et |
| CH₂CF₃ | CH(Bu-n)SPr-n |
| CH₂CF₃ | CH(Bu-n)S(O)Pr-n |
| CH₂CF₃ | CH(Bu-n)S(O)₂Pr-n |
| CH₂CF₃ | CH(Bu-n)SPr-i |
| CH₂CF₃ | CH(Bu-n)S(O)Pr-i |
| CH₂CF₃ | CH(Bu-n)S(O)₂Pr-i |
| CH₂CF₃ | CH(Bu-n)SPr-c |
| CH₂CF₃ | CH(Bu-n)S(O)Pr-c |
| CH₂CF₃ | CH(Bu-n)S(O)₂Pr-c |
| CH₂CF₃ | CH(Bu-n)SCH₂CF₃ |
| CH₂CF₃ | CH(Bu-n)S(O)CH₂CF₃ |
| CH₂CF₃ | CH(Bu-n)S(O)₂CH₂CF₃ |
| CH₂CF₃ | CH(F)SMe |
| CH₂CF₃ | CH(F)S(O)Me |
| CH₂CF₃ | CH(F)S(O)₂Me |
| CH₂CF₃ | CH(F)SEt |
| CH₂CF₃ | CH(F)S(O)Et |
| CH₂CF₃ | CH(F)S(O)₂Et |
| CH₂CF₃ | CH(F)S(═NCN)Et |
| CH₂CF₃ | CH(F)S(O)(═NCN)Et |
| CH₂CF₃ | CH(F)SCH₂CF₃ |
| CH₂CF₃ | CH(F)S(O)CH₂CF₃ |
| CH₂CF₃ | CH(F)S(O)₂CH₂CF₃ |
| CH₂CF₃ | C(F)₂SMe |
| CH₂CF₃ | C(F)₂S(O)Me |
| CH₂CF₃ | C(F)₂S(O)₂Me |
| CH₂CF₃ | C(F)₂SEt |
| CH₂CF₃ | C(F)₂S(O)Et |
| CH₂CF₃ | C(F)₂S(O)₂Et |
| CH₂CF₃ | C(F)₂S(═NCN)Et |
| CH₂CF₃ | C(F)₂S(O)(═NCN)Et |
| CH₂CF₃ | C(F)₂SCH₂CF₃ |
| CH₂CF₃ | C(F)₂S(O)CH₂CF₃ |
| CH₂CF₃ | C(F)₂S(O)₂CH₂CF₃ |
| CH₂CF₃ | CH(Cl)SMe |
| CH₂CF₃ | CH(Cl)S(O)Me |
| CH₂CF₃ | CH(Cl)S(O)₂Me |
| CH₂CF₃ | CH(Cl)SEt |
| CH₂CF₃ | CH(Cl)S(O)Et |
| CH₂CF₃ | CH(Cl)S(O)₂Et |
| CH₂CF₃ | CH(Cl)S(═NCN)Et |
| CH₂CF₃ | CH(Cl)S(O)(═NCN)Et |
| CH₂CF₃ | CH(Cl)SCH₂CF₃ |
| CH₂CF₃ | CH(Cl)S(O)CH₂CF₃ |
| CH₂CF₃ | CH(Cl)S(O)₂CH₂CF₃ |
| CH₂CF₃ | C(Cl)₂SMe |
| CH₂CF₃ | C(Cl)₂S(O)Me |
| CH₂CF₃ | C(Cl)₂S(O)₂Me |
| CH₂CF₃ | C(Cl)₂SEt |
| CH₂CF₃ | C(Cl)₂S(O)Et |
| CH₂CF₃ | C(Cl)₂S(O)₂Et |
| CH₂CF₃ | C(Cl)₂S(═NCN)Et |
| CH₂CF₃ | C(Cl)₂S(O)(═NCN)Et |
| CH₂CF₃ | C(Cl)₂SCH₂CF₃ |
| CH₂CF₃ | C(Cl)₂S(O)CH₂CF₃ |
| CH₂CF₃ | C(Cl)₂S(O)₂CH₂CF₃ |
| CH₂CF₃ | CH(Br)SMe |
| CH₂CF₃ | CH(Br)S(O)Me |
| CH₂CF₃ | CH(Br)S(O)₂Me |
| CH₂CF₃ | CH(Br)SEt |
| CH₂CF₃ | CH(Br)S(O)Et |
| CH₂CF₃ | CH(Br)S(O)₂Et |
| CH₂CF₃ | CH(Br)S(═NCN)Et |
| CH₂CF₃ | CH(Br)S(O)(═NCN)Et |
| CH₂CF₃ | CH(Br)SCH₂CF₃ |
| CH₂CF₃ | CH(Br)S(O)CH₂CF₃ |
| CH₂CF₃ | CH(Br)S(O)₂CH₂CF₃ |
| CH₂CF₃ | CH(I)SMe |
| CH₂CF₃ | CH(I)S(O)Me |
| CH₂CF₃ | CH(I)S(O)₂Me |
| CH₂CF₃ | CH(I)SEt |
| CH₂CF₃ | CH(I)S(O)Et |
| CH₂CF₃ | CH(I)S(O)₂Et |
| CH₂CF₃ | CH(I)S(═NCN)Et |
| CH₂CF3 | CH(I)S(O)(═NCN)Et |
| CH₂CF3 | CH(I)SCH₂CF₃ |
| CH₂CF₃ | CH(I)S(O)CH₂CF₃ |
| CH₂CF₃ | CH(I)S(O)₂CH₂CF₃ |
| CH₂CF₃ | CH{OC(O)Me}SMe |
| CH₂CF₃ | CH{OC(O)Me}S(O)Me |
| CH₂CF₃ | CH{OC(O)Me}S(O)₂Me |
| CH₂CF₃ | CH{OC(O)OMe}SEt |
| CH₂CF₃ | CH{OC(O)OMe}S(O)Et |
| CH₂CF₃ | CH{OC(O)OMe}S(O)₂Et |
| CH₂CF₃ | CH{OC(O)OMe}S(═NCN)Et |
| CH₂CF₃ | CH{OC(O)OMe}S(O)(═NCN)Et |
| CH₂CF₃ | CH{OC(O)OEt}SMe |
| CH₂CF₃ | CH{OC(O)OEt}S(O)Me |
| CH₂CF₃ | CH{OC(O)OEt}S(O)₂Me |
| CH₂CF₃ | CH{OC(O)OEt}SEt |
| CH₂CF₃ | CH{OC(O)OEt}S(O)Et |
| CH₂CF₃ | CH{OC(O)OEt}S(O)₂Et |
| CH₂CF₃ | CH{OC(O)OEt}S(═NCN)Et |
| CH₂CF₃ | CH{OC(O)OEt}S(O)(═NCN)Et |
| CH₂CF₃ | CH(OMe)SMe |
| CH₂CF₃ | CH(OMe)S(O)Me |
| CH₂CF₃ | CH(OMe)S(O)₂Me |
| CH₂CF₃ | CH(OMe)SEt |
| CH₂CF₃ | CH(OMe)S(O)Et |
| CH₂CF₃ | CH(OMe)S(O)₂Et |
| CH₂CF₃ | CH(OMe)S(═NCN)Et |
| CH₂CF₃ | CH(OMe)S(O)(═NCN)Et |
| CH₂CF₃ | CH(OEt)SMe |
| CH₂CF₃ | CH(OEt)S(O)Me |
| CH₂CF₃ | CH(OEt)S(O)₂Me |
| CH₂CF₃ | CH(OEt)SEt |
| CH₂CF₃ | CH(OEt)S(O)Et |
| CH₂CF₃ | CH(OEt)S(O)₂Et |
| CH₂CF₃ | CH(OEt)S(═NCN)Et |
| CH₂CF₃ | CH(OEt)S(O)(═NCN)Et |
| CH₂CF₃ | CH(SMe)₂ |
| CH₂CF₃ | CH(SMe)S(O)Me |
| CH₂CF₃ | CH(SMe)S(O)₂Me |
| CH₂CF₃ | CH(SMe)SEt |
| CH₂CF₃ | CH(SMe)S(O)Et |
| CH₂CF₃ | CH(SMe)S(O)₂Et |
| CH₂CF₃ | CH(SMe)S(═NCN)Et |
| CH₂CF₃ | CH(SMe)S(O)(═NCN)Et |
| CH₂CF₃ | CH(SEt)SMe |
| CH₂CF₃ | CH(SEt)S(O)Me |
| CH₂CF₃ | CH(SEt)S(O)₂Me |
| CH₂CF₃ | CH(OEt)SEt |
| CH₂CF₃ | CH(SEt)S(O)Et |
| CH₂CF₃ | CH(SEt)S(O)₂Et |
| CH₂CF₃ | CH(SEt)S(═NCN)Et |
| CH₂CF₃ | CH(SEt)S(O)(═NCN)Et |
| CH₂CF₃ | CH(CN)SMe |
| CH₂CF₃ | CH(CN)S(O)Me |
| CH₂CF₃ | CH(CN)S(O)₂Me |
| CH₂CF₃ | CH(CN)SEt |
| CH₂CF₃ | CH(CN)S(O)Et |
| CH₂CF₃ | CH(CN)S(O)₂Et |
| CH₂CF₃ | CH(CN)S(═NCN)Et |
| CH₂CF₃ | CH(CN)S(O)(═NCN)Et |
| CH₂CF₃ | CH{C(O)OMe}SMe |
| CH₂CF₃ | CH{C(O)OMe}S(O)Me |
| CH₂CF₃ | CH{C(O)OMe}S(O)₂Me |
| CH₂CF₃ | CH{C(O)OMe}SMe |
| CH₂CF₃ | CH{C(O)OMe}S(O)Me |
| CH₂CF₃ | CH{C(O)OMe}S(O)₂Me |
| CH₂CF₃ | CH{C(O)OMe}SEt |
| CH₂CF₃ | CH{C(O)OMe}S(O)Et |
| CH₂CF₃ | CH{C(O)OMe}S(O)₂Et |
| CH₂CF₃ | CH{C(O)OMe}S(═NCN)Et |
| CH₂CF₃ | CH{C(O)OMe}S(O)(═NCN)Et |
| CH₂CF₃ | CH{C(O)OEt}SMe |
| CH₂CF₃ | CH{C(O)OEt}S(O)Me |
| CH₂CF₃ | CH{C(O)OEt}S(O)₂Me |
| CH₂CF₃ | CH{C(O)OEt}SEt |
| CH₂CF₃ | CH{C(O)OEt}S(O)Et |
| CH₂CF₃ | CH{C(O)OEt}S(O)₂Et |
| CH₂CF₃ | CH{C(O)OEt}S(═NCN)Et |
| CH₂CF₃ | CH{C(O)OEt}S(O)(═NCN)Et |
| CH₂CF₃ | C(Me)₂SMe |
| CH₂CF₃ | C(Me)₂S(O)Me |
| CH₂CF₃ | C(Me)₂S(O)₂Me |
| CH₂CF₃ | OBu-t |
| S(O)₂Me | CH₂SMe |
| S(O)₂Me | CH₂S(O)Me |
| S(O)₂Me | CH₂S(O)₂Me |
| S(O)₂Me | CH₂SEt |
| S(O)₂Me | CH₂S(O)Et |
| S(O)₂Me | CH₂S(O)₂Et |
| S(O)₂Me | CH₂SPr-n |
| S(O)₂Me | CH₂S(O)Pr-n |
| S(O)₂Me | CH₂S(O)₂Pr-n |
| S(O)₂Me | CH₂SPr-i |
| S(O)₂Me | CH₂S(O)Pr-i |
| S(O)₂Me | CH₂S(O)₂Pr-i |
| S(O)₂Me | CH₂SPr-c |
| S(O)₂Me | CH₂S(O)Pr-c |
| S(O)₂Me | CH₂S(O)₂Pr-c |
| S(O)₂Me | CH₂SCH₂CH═CH₂ |
| S(O)₂Me | CH₂S(O)CH₂CH═CH₂ |
| S(O)₂Me | CH₂S(O)₂CH₂CH═CH₂ |
| S(O)₂Me | CH₂SCH₂C≡CH |
| S(O)₂Me | CH₂S(O)CH₂C≡CH |
| S(O)₂Me | CH₂S(O)₂CH₂C≡CH |
| S(O)₂Me | CH₂SCH₂CF₃ |
| S(O)₂Me | CH₂S(O)CH₂CF₃ |
| S(O)₂Me | CH₂S(O)₂CH₂CF₃ |
| S(O)₂Me | CH₂SCH₂CH(OMe)₂ |
| S(O)₂Me | CH₂SCH₂CH(═NOMe) |
| S(O)₂Me | CH(Me)SMe |
| S(O)₂Me | CH(Me)S(O)Me |
| S(O)₂Me | CH(Me)S(O)₂Me |
| S(O)₂Me | CH(Me)SEt |
| S(O)₂Me | CH(Me)S(O)Et |
| S(O)₂Me | CH(Me)S(O)₂Et |
| S(O)₂Me | CH(Me)S(═NCN)Et |
| S(O)₂Me | CH(Me)S(O)(═NCN)Et |
| S(O)₂Me | CH(Me)SPr-n |
| S(O)₂Me | CH(Me)S(O)Pr-n |
| S(O)₂Me | CH(Me)S(O)₂Pr-n |
| S(O)₂Me | CH(Me)SPr-i |
| S(O)₂Me | CH(Me)S(O)Pr-i |
| S(O)₂Me | CH(Me)S(O)₂Pr-i |
| S(O)₂Me | CH(Me)SBu-t |
| S(O)₂Me | CH(Me)S(O)Bu-t |
| S(O)₂Me | CH(Me)S(O)₂Bu-t |
| S(O)₂Me | CH(Me)SCH₂Pr-c |
| S(O)₂Me | CH(Me)S(O)CH₂Pr-c |
| S(O)₂Me | CH(Me)S(O)₂CH₂Pr-c |
| S(O)₂Me | CH(Me)SCH₂OMe |
| S(O)₂Me | CH(Me)SCH₂SMe |
| S(O)₂Me | CH(Me)S(O)CH₂SMe |
| S(O)₂Me | CH(Me)S(O)₂CH₂SMe |
| S(O)₂Me | CH(Me)SCH₂C═CH₂ |
| S(O)₂Me | CH(Me)S(O)CH₂C═CH₂ |
| S(O)₂Me | CH(Me)S(O)₂CH₂C═CH₂ |
| S(O)₂Me | CH(Me)SCH₂C≡CH |
| S(O)₂Me | CH(Me)S(O)CH₂C≡CH |
| S(O)₂Me | CH(Me)S(O)₂CH₂C≡CH |
| S(O)₂Me | CH(Me)SCH₂C(O)NHMe |
| S(O)₂Me | CH(Me)S(O)CH₂C(O)NHMe |
| S(O)₂Me | CH(Me)S(O)₂CH₂C(O)NHMe |
| S(O)₂Me | CH(Me)SCH₂C(O)OMe |
| S(O)₂Me | CH(Me)S(O)CH₂C(O)OMe |
| S(O)₂Me | CH(Me)S(O)₂CH₂C(O)OMe |
| S(O)₂Me | CH(Me)SCH₂CH₂CH₂Cl |
| S(O)₂Me | CH(Me)S(O)CH₂CH₂CH₂Cl |
| S(O)₂Me | CH(Me)S(O)₂CH₂CH₂CH₂Cl |
| S(O)₂Me | CH(Me)SCH₂CF₃ |
| S(O)₂Me | CH(Me)S(O)CH₂CF₃ |
| S(O)₂Me | CH(Me)S(O)₂CH₂CF₃ |
| S(O)₂Me | CH(Me)SCH₂Ph |
| S(O)₂Me | CH(Me)S(O)CH₂Ph |
| S(O)₂Me | CH(Me)S(O)₂CH₂Ph |
| S(O)₂Me | CH(Me)SCH₂(D1-34a) |
| S(O)₂Me | CH(Me)S(O)CH₂(D1-34a) |
| S(O)₂Me | CH(Me)S(O)₂CH₂(D1-34a) |
| S(O)₂Me | CH(CH₃)SCH₂Si(CH₃)₃ |
| S(O)₂Me | CH(Me)SCN |
| S(O)₂Me | CH(Me)SCH₂CN |
| S(O)₂Me | CH(Me)SC(O)Me |
| S(O)₂Me | CH(Me)S{D1-12a(X^{1a}═Me)} |
| S(O)₂Me | CH(Me)S(D1-32a) |
| S(O)₂Me | CH(Me)S{D1-32b(3-NO₂)} |
| S(O)₂Me | CH(Me)S{D1-32b(3-CF₃)} |
| S(O)₂Me | CH(Me)S(D1-37a) |
| S(O)₂Me | CH(Me)S(D1-51a) |
| S(O)₂Me | CH(Et)SMe |
| S(O)₂Me | CH(Et)S(O)Me |
| S(O)₂Me | CH(Et)S(O)₂Me |
| S(O)₂Me | CH(Et)SEt |
| S(O)₂Me | CH(Et)S(O)Et |
| S(O)₂Me | CH(Et)S(O)₂Et |
| S(O)₂Me | CH(Et)SCH₂CF₃ |
| S(O)₂Me | CH(Et)S(O)CH₂CF₃ |
| S(O)₂Me | CH(Et)S(O)₂CH₂CF₃ |
| S(O)₂Me | CH(Pr-n)SMe |
| S(O)₂Me | CH(Pr-n)S(O)Me |
| S(O)₂Me | CH(Pr-n)S(O)₂Me |
| S(O)₂Me | CH(Pr-n)SEt |
| S(O)₂Me | CH(Pr-n)S(O)Et |
| S(O)₂Me | CH(Pr-n)S(O)₂Et |
| S(O)₂Me | CH(Pr-n)S(═NCN)Et |
| S(O)₂Me | CH(Pr-n)S(O)(═NCN)Et |
| S(O)₂Me | CH(Pr-n)SPr-n |
| S(O)₂Me | CH(Pr-n)S(O)Pr-n |
| S(O)₂Me | CH(Pr-n)S(O)₂Pr-n |
| S(O)₂Me | CH(Pr-n)SPr-i |
| S(O)₂Me | CH(Pr-n)S(O)Pr-i |
| S(O)₂Me | CH(Pr-n)S(O)₂Pr-i |
| S(O)₂Me | CH(Pr-n)SPr-c |
| S(O)₂Me | CH(Pr-n)S(O)Pr-c |
| S(O)₂Me | CH(Pr-n)S(O)₂Pr-c |
| S(O)₂Me | CH(Pr-n)SCH₂CF₃ |
| S(O)₂Me | CH(Pr-n)S(O)CH₂CF₃ |
| S(O)₂Me | CH(Pr-n)S(O)₂CH₂CF₃ |
| S(O)₂Me | CH(Pr-i)SMe |
| S(O)₂Me | CH(Pr-i)S(O)Me |
| S(O)₂Me | CH(Pr-i)S(O)₂Me |
| S(O)₂Me | CH(Pr-i)SEt |
| S(O)₂Me | CH(Pr-i)S(O)Et |
| S(O)₂Me | CH(Pr-i)S(O)₂Et |
| S(O)₂Me | CH(Pr-i)S(═NCN)Et |
| S(O)₂Me | CH(Pr-i)S(O)(═NCN)Et |
| S(O)₂Me | CH(Pr-i)SPr-n |
| S(O)₂Me | CH(Pr-i)S(O)Pr-n |
| S(O)₂Me | CH(Pr-i)S(O)₂Pr-n |
| S(O)₂Me | CH(Pr-i)SPr-i |
| S(O)₂Me | CH(Pr-i)S(O)Pr-i |
| S(O)₂Me | CH(Pr-i)S(O)₂Pr-i |
| S(O)₂Me | CH(Pr-i)SPr-c |
| S(O)₂Me | CH(Pr-i)S(O)Pr-c |
| S(O)₂Me | CH(Pr-i)S(O)₂Pr-c |
| S(O)₂Me | CH(Pr-i)SCH₂CF₃ |
| S(O)₂Me | CH(Pr-i)S(O)CH₂CF₃ |
| S(O)₂Me | CH(Pr-i)S(O)₂CH₂CF₃ |
| S(O)₂Me | CH(Pr-c)SMe |
| S(O)₂Me | CH(Pr-c)S(O)Me |
| S(O)₂Me | CH(Pr-c)S(O)₂Me |
| S(O)₂Me | CH(Pr-c)SEt |
| S(O)₂Me | CH(Pr-c)S(O)Et |
| S(O)₂Me | CH(Pr-c)S(O)₂Et |
| S(O)₂Me | CH(Pr-c)S(═NCN)Et |
| S(O)₂Me | CH(Pr-c)S(O)(═NCN)Et |
| S(O)₂Me | CH(Pr-c)SPr-n |
| S(O)₂Me | CH(Pr-c)S(O)Pr-n |
| S(O)₂Me | CH(Pr-c)S(O)₂Pr-n |
| S(O)₂Me | CH(Pr-c)SPr-i |
| S(O)₂Me | CH(Pr-c)S(O)Pr-i |
| S(O)₂Me | CH(Pr-c)S(O)₂Pr-i |
| S(O)₂Me | CH(Pr-c)SPr-c |
| S(O)₂Me | CH(Pr-c)S(O)Pr-c |
| S(O)₂Me | CH(Pr-c)S(O)₂Pr-c |
| S(O)₂Me | CH(Pr-c)SCH₂CF₃ |
| S(O)₂Me | CH(Pr-c)S(O)CH₂CF₃ |
| S(O)₂Me | CH(Pr-c)S(O)₂CH₂CF₃ |
| S(O)₂Me | CH(Bu-n)SMe |
| S(O)₂Me | CH(Bu-n)S(O)Me |
| S(O)₂Me | CH(Bu-n)S(O)₂Me |
| S(O)₂Me | CH(Bu-n)SEt |
| S(O)₂Me | CH(Bu-n)S(O)Et |
| S(O)₂Me | CH(Bu-n)S(O)₂Et |
| S(O)₂Me | CH(Bu-n)S(═NCN)Et |
| S(O)₂Me | CH(Bu-n)S(O)(═NCN)Et |
| S(O)₂Me | CH(Bu-n)SPr-n |
| S(O)₂Me | CH(Bu-n)S(O)Pr-n |
| S(O)₂Me | CH(Bu-n)S(O)₂Pr-n |
| S(O)₂Me | CH(Bu-n)SPr-i |
| S(O)₂Me | CH(Bu-n)S(O)Pr-i |
| S(O)₂Me | CH(Bu-n)S(O)₂Pr-i |
| S(O)₂Me | CH(Bu-n)SPr-c |
| S(O)₂Me | CH(Bu-n)S(O)Pr-c |
| S(O)₂Me | CH(Bu-n)S(O)₂Pr-c |
| S(O)₂Me | CH(Bu-n)SCH₂CF₃ |
| S(O)₂Me | CH(Bu-n)S(O)CH₂CF₃ |
| S(O)₂Me | CH(Bu-n)S(O)₂CH₂CF₃ |
| S(O)₂Me | CH(F)SMe |
| S(O)₂Me | CH(F)S(O)Me |
| S(O)₂Me | CH(F)S(O)₂Me |
| S(O)₂Me | CH(F)SEt |
| S(O)₂Me | CH(F)S(O)Et |
| S(O)₂Me | CH(F)S(O)₂Et |
| S(O)₂Me | CH(F)S(═NCN)Et |
| S(O)₂Me | CH(F)S(O)(═NCN)Et |
| S(O)₂Me | CH(F)SCH₂CF₃ |
| S(O)₂Me | CH(F)S(O)CH₂CF₃ |
| S(O)₂Me | CH(F)S(O)₂CH₂CF₃ |
| S(O)₂Me | C(F)₂SMe |
| S(O)₂Me | C(F)₂S(O)Me |
| S(O)₂Me | C(F)₂S(O)₂Me |
| S(O)₂Me | C(F)₂SEt |
| S(O)₂Me | C(F)₂S(O)Et |
| S(O)₂Me | C(F)₂S(O)₂Et |
| S(O)₂Me | C(F)₂S(═NCN)Et |
| S(O)₂Me | C(F)₂S(O)(═NCN)Et |
| S(O)₂Me | C(F)₂SCH₂CF₃ |
| S(O)₂Me | C(F)₂S(O)CH₂CF₃ |
| S(O)₂Me | C(F)₂S(O)₂CH₂CF₃ |
| S(O)₂Me | CH(Cl)SMe |
| S(O)₂Me | CH(Cl)S(O)Me |
| S(O)₂Me | CH(Cl)S(O)₂Me |
| S(O)₂Me | CH(Cl)SEt |
| S(O)₂Me | CH(Cl)S(O)Et |
| S(O)₂Me | CH(Cl)S(O)₂Et |
| S(O)₂Me | CH(Cl)S(═NCN)Et |
| S(O)₂Me | CH(Cl)S(O)(═NCN)Et |
| S(O)₂Me | CH(Cl)SCH₂CF₃ |
| S(O)₂Me | CH(Cl)S(O)CH₂CF₃ |
| S(O)₂Me | CH(Cl)S(O)₂CH₂CF₃ |
| S(O)₂Me | C(Cl)₂SMe |
| S(O)₂Me | C(Cl)₂S(O)Me |
| S(O)₂Me | C(Cl)₂S(O)₂Me |
| S(O)₂Me | C(Cl)₂SEt |
| S(O)₂Me | C(Cl)₂S(O)Et |
| S(O)₂Me | C(Cl)₂S(O)₂Et |
| S(O)₂Me | C(Cl)₂S(═NCN)Et |
| S(O)₂Me | C(Cl)₂S(O)(═NCN)Et |
| S(O)₂Me | C(Cl)₂SCH₂CF₃ |
| S(O)₂Me | C(Cl)₂S(O)CH₂CF₃ |
| S(O)₂Me | C(Cl)₂S(O)₂CH₂CF₃ |
| S(O)₂Me | CH(Br)SMe |
| S(O)₂Me | CH(Br)S(O)Me |
| S(O)₂Me | CH(Br)S(O)₂Me |
| S(O)₂Me | CH(Br)SEt |
| S(O)₂Me | CH(Br)S(O)Et |
| S(O)₂Me | CH(Br)S(O)₂Et |
| S(O)₂Me | CH(Br)S(═NCN)Et |
| S(O)₂Me | CH(Br)S(O)(═NCN)Et |
| S(O)₂Me | CH(Br)SCH₂CF₃ |
| S(O)₂Me | CH(Br)S(O)CH₂CF₃ |
| S(O)₂Me | CH(Br)S(O)₂CH₂CF₃ |
| S(O)₂Me | CH(I)SMe |
| S(O)₂Me | CH(I)S(O)Me |
| S(O)₂Me | CH(I)S(O)₂Me |
| S(O)₂Me | CH(I)SEt |
| S(O)₂Me | CH(I)S(O)Et |
| S(O)₂Me | CH(I)S(O)₂Et |
| S(O)₂Me | CH(I)S(═NCN)Et |
| S(O)₂Me | CH(I)S(O)(═NCN)Et |
| S(O)₂Me | CH(I)SCH₂CF₃ |
| S(O)₂Me | CH(I)S(O)CH₂CF₃ |
| S(O)₂Me | CH(I)S(O)₂CH₂CF₃ |
| S(O)₂Me | CH{OC(O)Me}SMe |
| S(O)₂Me | CH{OC(O)Me}S(O)Me |
| S(O)₂Me | CH{OC(O)Me}S(O)₂Me |
| S(O)₂Me | CH{OC(O)OMe}SEt |
| S(O)₂Me | CH{OC(O)OMe}S(O)Et |
| S(O)₂Me | CH{OC(O)OMe}S(O)₂Et |
| S(O)₂Me | CH{OC(O)OMe}S(═NCN)Et |
| S(O)₂Me | CH{OC(O)OMe}S(O)(═NCN)Et |
| S(O)₂Me | CH{OC(O)OEt}SMe |
| S(O)₂Me | CH{OC(O)OEt}S(O)Me |
| S(O)₂Me | CH{OC(O)OEt}S(O)₂Me |
| S(O)₂Me | CH{OC(O)OEt}SEt |
| S(O)₂Me | CH{OC(O)OEt}S(O)Et |
| S(O)₂Me | CH{OC(O)OEt}S(O)₂Et |
| S(O)₂Me | CH{OC(O)OEt}S(═NCN)Et |
| S(O)₂Me | CH{OC(O)OEt}S(O)(═NCN)Et |
| S(O)₂Me | CH(OMe)SMe |
| S(O)₂Me | CH(OMe)S(O)Me |
| S(O)₂Me | CH(OMe)S(O)₂Me |
| S(O)₂Me | CH(OMe)SEt |
| S(O)₂Me | CH(OMe)S(O)Et |
| S(O)₂Me | CH(OMe)S(O)₂Et |
| S(O)₂Me | CH(OMe)S(═NCN)Et |
| S(O)₂Me | CH(OMe)S(O)(═NCN)Et |
| S(O)₂Me | CH(OEt)SMe |
| S(O)₂Me | CH(OEt)S(O)Me |
| S(O)₂Me | CH(OEt)S(O)₂Me |
| S(O)₂Me | CH(OEt)SEt |
| S(O)₂Me | CH(OEt)S(O)Et |
| S(O)₂Me | CH(OEt)S(O)₂Et |
| S(O)₂Me | CH(OEt)S(═NCN)Et |
| S(O)₂Me | CH(OEt)S(O)(═NCN)Et |
| S(O)₂Me | CH(SMe)₂ |
| S(O)₂Me | CH(SMe)S(O)Me |
| S(O)₂Me | CH(SMe)S(O)₂Me |
| S(O)₂Me | CH(SMe)SEt |
| S(O)₂Me | CH(SMe)S(O)Et |
| S(O)₂Me | CH(SMe)S(O)₂Et |
| S(O)₂Me | CH(SMe)S(═NCN)Et |
| S(O)₂Me | CH(SMe)S(O)(═NCN)Et |
| S(O)₂Me | CH(SEt)SMe |
| S(O)₂Me | CH(SEt)S(O)Me |
| S(O)₂Me | CH(SEt)S(O)₂Me |
| S(O)₂Me | CH(SEt)SEt |
| S(O)₂Me | CH(SEt)S(O)Et |
| S(O)₂Me | CH(SEt)S(O)₂Et |
| S(O)₂Me | CH(SEt)S(═NCN)Et |
| S(O)₂Me | CH(SEt)S(O)(═NCN)Et |
| S(O)₂Me | CH(CN)SMe |
| S(O)₂Me | CH(CN)S(O)Me |
| S(O)₂Me | CH(CN)S(O)₂Me |
| S(O)₂Me | CH(CN)SEt |
| S(O)₂Me | CH(CN)S(O)Et |
| S(O)₂Me | CH(CN)S(O)₂Et |
| S(O)₂Me | CH(CN)S(═NCN)Et |
| S(O)₂Me | CH(CN)S(O)(═NCN)Et |
| S(O)₂Me | CH{C(O)OMe}SMe |
| S(O)₂Me | CH{C(O)OMe}S(O)Me |
| S(O)₂Me | CH{C(O)OMe}S(O)₂Me |
| S(O)₂Me | CH{C(O)OMe}SMe |
| S(O)₂Me | CH{C(O)OMe}S(O |
| S(O)₂Me | CH{C(O)OMe}S(O)₂Me |
| S(O)₂Me | CH{C(O)OMe}SEt |
| S(O)₂Me | CH{C(O)OMe}S(O)Et |
| S(O)₂Me | CH{C(O)OMe}S(O)₂Et |
| S(O)₂Me | CH{C(O)OMe}S(═NCN)Et |
| S(O)₂Me | CH{C(O)OMe}S(O)(═NCN)Et |
| S(O)₂Me | CH{C(O)OEt}SMe |
| S(O)₂Me | CH{C(O)OEt}S(O)Me |
| S(O)₂Me | CH{C(O)OEt}S(O)₂Me |
| S(O)₂Me | CH{C(O)OEt}SEt |
| S(O)₂Me | CH{C(O)OEt}S(O)Et |
| S(O)₂Me | CH{C(O)OEt}S(O)₂Et |
| S(O)₂Me | CH{C(O)OEt}S(═NCN)Et |
| S(O)₂Me | CH{C(O)OEt}S(O)(═NCN)Et |
| S(O)₂Me | C(Me)₂SMe |
| S(O)₂Me | C(Me)₂S(O)Me |
| S(O)₂Me | C(Me)₂S(O)₂Me |
| SC(Cl)₃ | CH(Me)SMe |
| SC(Cl)₃ | CH(Me)S(O)Me |
| SC(Cl)₃ | CH(Me)S(O)₂Me |
| SC(Cl)₃ | CH(Me)SEt |
| SC(Cl)₃ | CH(Me)S(O)Et |
| SC(Cl)₃ | CH(Me)S(O)₂Et |
| SC(Cl)₃ | CH(Me)CH₂SMe |
| CH₂CH₂CH₂SMe | Me |
| CH₂CH₂SMe | Me |
| NH₂ | OBu-t |
| NHC(O)CH₂SMe | OBu-t |
| N(Me)C(O)CH₂SMe | OBu-t |
| NHC(O)CH(Me)SMe | OBu-t |
| N(Me)C(O)CH(Me)SMe | OBu-t |
| NHC(O)CH₂CH₂SMe | OBu-t |
| N{C(O)OBu-t}₂ | OBu-t |
| NHC(O)OBu-t | OBu-t |

—CH₂CH₂CH₂—
—CH₂CH₂C(═CH₂)—
—CH₂CH₂CH(CH₂SMe)—
—CH₂CH₂CH{CH₂S(O)Me}—
—CH₂CH₂CH{CH₂S(O)₂Me}—
—CH₂CH₂CH₂CH₂—
—CH₂CH₂CH═CH—
—CH₂CH═CHCH₂—
—CH₂CH₂CH₂CH(Br)—
—CH₂CH₂CH₂CH(SCH₃)—
—CH₂CH₂CH₂CH{S(O)CH₃}—
—CH₂CH₂CH₂CH{S(O)₂CH₃}—
—CH₂CH₂CH₂C(F){S(O)₂CH₃}—
—CH₂CH₂CH(SCH₃)CH₂—
—CH₂CH₂CH₂CH(CH₂SCH₃)—
—CH₂CH₂CH(CH₂SCH₃)CH₂—
—CH₂CH₂CH₂CH(OCH₂CF₃)—
—CH₂CH₂CH(OCH₂CF₃)CH₂—
—CH₂CH₂SCH₂—
—CH₂OCH(CF₃)CH₂—
—CH₂OCH₂N(CH₂CF₃)—
—CH₂OCH₂N(CH₂SMe)—
—CH₂CH₂N(CH₂CF₃)—
—CH₂CH₂N(CH₂SMe)—
—C(O)CH₂CH₂—
—C(O)CH═C(Me)—
—C(O)CH₂C(═CH₂)—
—C(O)NHCH(CH₂SMe)—
—C(O)NHCH{CH₂S(O)Me}—
—C(O)NHCH{CH2S(O)₂Me}—
—CH₂C(OCH₃)₂—

TABLE 3

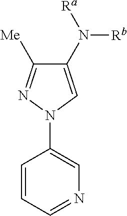

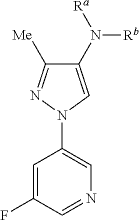

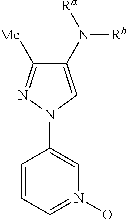

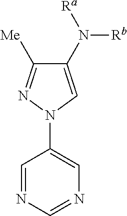

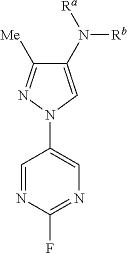

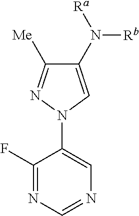

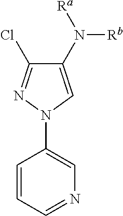

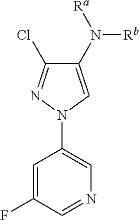

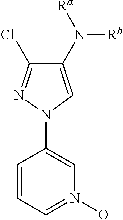

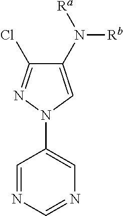

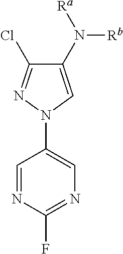

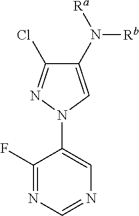

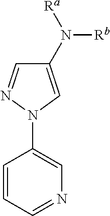

| R^{a} | | R^{b} |
|---|---|---|
| H | | D1-51a |
| Me | | D1-51a |
| Et | | D1-51a |
| | ═C(Me)OMe | |
| | ═C(Me)OEt | |
| | ═CHNMe₂ | |
| | ═C(Me)N(Me)C(O)CH₂SMe | |
| | ═C(SMe)CH(Me)SEt | |
| | —C(═NOMe)CH₂CH₂CH₂— | |
| | D1-88b{X^{1a} = C(O)CH₂SCH₃} | |

The compounds of the present invention can effectively control insects including so-called agricultural pest insects damaging agricultural crops and trees, so-called livestock pest insects which parasitize livestock and poultry, so-called hygienic insects having harmful effects on houses and living environments and so-called grain-storage insects damaging grains stored in warehouses and any pests which live and give damage in similar settings such as mites, crustaceans, mollusks and nematodes at low doses. The insects, mites, crustaceans, mollusks and nematodes that the compounds of the present invention can control include the following organisms, but the present invention is not restricted thereto.

Insects of the order Lepidoptera such as Adoxophyes honmai, Adoxophyes orana faciata, Archips breviplicanus, Archips fuscocupreanus, Grapholita molesta, Homona magnanima, Leguminivora glycinivorella, Matsumuraeses phaseoli, Pandemis heparana, Bucculatrix pyrivorella, Lyonetia clerkella, Lyonetia prunifoliella malinella, Caloptilia theivora, Phyllonorycter ringoniella, Phyllocnistis citrella, Acrolepiopsis sapporensis, Acrolepiopsis suzukiella, Plutella xylostella, Stathmopoda masinissa, Helcystogramma triannulella, Pectinophora cossypiella, Carposina sasakii, Cydla pomonella, Chilo suppressalis, Cnaphalocrocis medinalis, Conogethes punctiferalis, Diaphania indica, Etiella zinckenella, Glyphodes pyloalis, Hellula undalis, Ostrinia furnacalis, Ostrinia scapulalis, Ostrinia nubilalis, Parapediasia teterrella, Parnara guttata, Pieris brassicae, Pieris rapae crucivora, Ascotis selenaria, Pseudoplusia includens, Euproctis pseudoconspersa, Lymantria dispar, Orgyia thyellina, Hyphantria cunea, Lemyra imparilis, Adris tyrannus, Aedia leucomelas, Agrotis ipsilon, Agrotis segetum, Autographa nigrisigna, Ctenoplusia agnata, Helicoverpa armigera, Helicoverpa assulta, Helicoverpa zea, Heliothis virescens, Mamestra brassicae, Mythimna separata, Naranga aenescens, Spodoptera eridania, Spodoptera exigua, Spodoptera frugiperda, Spodoptera littoralis, Spodoptera litura, Spodoptera depravata, Trichoplusia ni, Endopiza viteana, Manduca quinquemaculata and Manduca sexta.

Insects of the order Thysanoptera such as Frankliniella intonsa, Frankliniella occidentalis, Heliothrips haemorrhoidalis, Scirtothrips dorsalis, Thrips palmi, Thrips tabaci and Ponticulothrips diospyrosi.

Insects of the order Hemiptera such as Dolycoris baccarum, Eurydema rugosum, Eysarcoris aeneus, Eysarcoris lewisi, Eysarcoris ventralis, Glaucias subpunctatus, Halyomorpha halys, Nezara antennata, Nezara viridula, Piezodorus hybneri, Plautia crossota, Scotinophora lurida, Cletus punctiger, Leptocorisa chinensis, Riptortus clavatus, Rhopalus msculatus, Cavelerius saccharivorus, Togo hemipterus, Dysdercus cingulatus, Stephanitis pyrioides, Halticus insularis, Lygus lineolaris, Stenodema sibiricum, Stenotus rubrovittatus, Trigonotylus caelestialium, Arboridia apicalis, Balclutha saltuella, Epiacanthus stramineus, Empoasca fabae, Empoasca nipponica, Empoasca onukii, Empoasca sakaii, Macrosteles striifrons, Nephotettix cinctinceps, Psuedatomoscelis seriatus, Laodelphax striatella, Nilaparvata lugens, Sogatella furcifera, Diaphorina citri, Psylla pyrisuga, Aleurocanthus spiniferus, Bemisia argentifolii, Bemisia tabaci, Dialeurodes citri, Trialeurodes vaporariorum, Viteus vitifolii, Aphis gossypii, Aphis spiraecola, Myzus persicae, Toxoptera aurantii, Drosicha corpulenta, Icerya purchasi, Phenacoccus solani, Planococcus citri, Planococcus kuraunhiae, Pseudococcus comstocki, Ceroplastes ceriferus, Ceroplastes rubens, Aonidiella aurantii, Comstockaspis perniciosa, Fiorinia theae, Pseudaonidia paeoniae, Pseudaulacaspis pentagona, Pseudaulacaspis prunicola, Unaspis euonymi, Unaspis yanonensis and Cimex lectularius.

Insects of the order Coleoptera such as Anomala cuprea, Anomala rufocuprea, Gametis iucunda, Heptophylla picea, Popillia japonica, Lepinotarsa decemlineata, Melanotus fortnumi, Melanotus tamsuyensis, Lasioderma serricorne, Epuraea domina, Epilachna varivestis, Epilachna vigintioctopunctata, Tenebrio molitor, Tribolium castaneum, Anoplophora malasiaca, Monochamus alternatus, Psacothea hilaris, Xylotrechus pyrrhoderus, Callosobruchus chinensis, Aulacophora femoralis, Chaetocnema concinna, Diabrotica undecimpunctata, Diabrotica virgifera, Diabrotica barberi, Oulema oryzae, Phyllotreta striolata, Psylliodes angusticollis, Rhynchites heros, Cylas formicarius, Anthonomus grandis, Echinocnemus squameus, Euscepes postfasciatus, Hypera postica, Lissohoptrus oryzophilus, Otiorhynchus sulcatus, Sitophilus granarius, Sitophilus zeamais, Sphenophorus venatus vestitus and Paederus fuscipes.

Insects of the order Diptera such as Asphondylia yushimai, Sitodiplosis mosellana, Bactrocera cucurbitae, Bactrocera dorsalis, Ceratitis capitata, Hydrellia griseola, Drosophila suzukii, Agromyza oryzae, Chromatomyia horticola, Liriomyza bryoniae, Liriomyza chinensis, Liriomyza sativae, Liriomyza trifolii, Delia platura, Pegomya cunicularia, Rhagoletis pomonella, Mayetiola destructor, Musca domestica, Stomoxys calcitrans, Melophagus ovinus, Hypoderma bovis, Hypoderma lineatum, Oestrus ovis, Glossina palpalis, Glossina morsitans, Prosimulium yezoensis, Tabanus trigonus, Telmatoscopus albipunctatus, Leptoconops nipponensis, Culex pipiens pallens, Aedes aegypti, Aedes albopicutus and Anopheles hyracanus sinesis.

Insects of the order Hymenoptera such as Apethymus kuri, Athalia rosae, Arge pagana, Neodiprion sertifer, Dryocosmus kuriphilus, Eciton burchelli, Eciton schmitti, Camponotus iaponicus, Vespa mandarina, Myrmecia spp., Solenopsis spp. and Monomorium pharaonis.

Insects of the order Orthoptera such as Teleogryllus emma, Gryllotalpa orientalis, Locusta migratoria, Oxya yezoensis and Schistocerca gregaria.

Insects of the order Collembola such as Onychiurus folsomi, Onychiurus sibiricus and Bourletiella hortensis.

Insects of the order Dyctyoptera such as Periplaneta fuliginosa, Periplaneta iaponica and Blattella germanica.

Insects of the order Isoptera such as Coptotermes formosanus, Reticulitermes speratus and Odontotermes formosanus.

Insects of the order Isoptera such as Ctenocephalidae felis, Ctenocephalides canis, Echidnophaga gallinacea, Pulex irritans and Xenopsylla cheopis.

Insects of the order Mallophaga such as Menacanthus stramineus and Bovicola bovis.

Insects of the order Anoplura such as Haematopinus eurysternus, Haematopinus suis, Linognathus vituli and Solenopotes capillatus.

Tarsonemids such as Phytonemus pallidus, Polyphagotarsonemus latus and Tarsonemus bilobatus.

Eupodidae mites such as Penthaleus erythrocephalus and Penthaleus major.

Tetranychids such as Oligonychus shinkajii, Panonychus citri, Panonychus mori, Panonychus ulmi, Tetranychus kanzawai and Tetranychus urticae.

Eriophyids such as Acaphylla theavagrans, Aceria tulipae, Aculops lycopersici, Aculops pelekassi, Aculus schlechtendali, Eriophyes chibaensis and Phyllocoptruta oleivora.

Acarids such as Rhizoglyphus robini, Tyrophagus putrescentiae and Tyrophagus similis.

Bee mites such as Varroa jacobsoni.

Ticks such as Boophilus microplus, Rhipicephalus sanguineus, Haemaphysalis longicornis, Haemophysalis flava, Haemophysalis campanulata, Ixodes ovatus, Ixodes persulcatus, Amblyomma spp. and Dermacentor spp.

Cheyletids such as Cheyletiella vasguri and Cheyletiella blakei.

Demodicids such as Demodex canis and Demodex cati.

Psoroptic mites such as Psoroptes ovis.

Sarcoptes mites such as Sarcoptes scabiei, Notoedres cati and Knemidocoptes spp.

Crustaceans such as Armadillidium vulgare.

Gastropods such as Pomacea canaliculata, Achatina fulica, Meghimatium bilineatum, Limax Valentiana, Acusta despecta sieboldiana and Euhadra peliomphala.

Nematodes such as Prathylenchus coffeae, Prathylenchus penetrans, Prathylenchus vulnus, Globodera rostochiensis, Heterodera lycines, Meloidogyne hapla, Meloidogyne incognita, Aphelenchoides besseyi and Bursaphelenchus xylophilus.

The internal, livestock, poultry or pet parasites that the compounds of the present invention can control include the following organisms, but the present invention is not restricted thereto.

Nematodes of the genera Haemonchus, Trichostrongylus, Ostertagia, Nematodirus, Cooperia, Ascaris, Bunostomum, Oesophagostomum, Chabertia, Trichuris, Storongylus, Trichonema, Dictyocaulus, Capillaria, Heterakis, Toxocara, Ascaridia, Oxyuris, Ancylostoma, Uncinaria, Toxascaris, Parascaris, and the like.

Nematodes of the family Filariidae such as the genera Wuchereria, Brugia, Onchoceca, Dirofilaria, Loa. and the like.

Nematodes of the family Dracunculidae such as the genus Deacunculus.

Cestodes such as Dipylidium caninum, Taenia taeniaeformis, Taenia solium, Taenia saginata, Hymenolepis diminuta, Moniezia benedeni, Diphyllobothrium latum, Diphyllobothrium erinacei, Echinococcus granulosus and Echinococcus multilocularis.

Trematodes such as Fasciola hepatica, F. gigantica, Paragonimus westermanii, Fasciolopsic bruski, Eurytrema pancreaticum, E. coelomaticum, Clonorchis sinensis, Schistosoma japonicum, Schistosoma haematobium and Schistosoma mansoni.

Eimeria spp. such as Eimeria tenella, Eimeria acervulina, Eimeria brunetti, Eimeria maxima, Eimeria necatrix, Eimeria bovis and Eimeria ovinoidalis.

Trypanosomsa cruzi, Leishmania spp., Plasmodium spp., Babesis spp., Trichomonadidae spp., Histomanas spp., Giardia spp., Toxoplasma spp., Entamoeba histolytica and Theileria spp.

The compounds of the present invention are effective against pests that have acquired resistance to conventional insecticides such as organic phosphorus compounds, carbamate compounds or pyrethroid compounds.

Namely, the compounds of the present invention can effectively control pests such as insects of the order Collembola, the order Dyctyoptera, the order Orthoptera, the order Isoptera, the order Thysanoptera, the order Hemiptera, the order Lepidoptera, the order Coleoptera, the order Hymenoptera, the order Diptera, the order Aphaniptera, the order Anoplura, mites, gastropods and nematodes at low doses. On the other hand, the compounds of the present invention have a quite advantageous feature that they are almost harmless to mammals, fishes, crustaceans and beneficial insects (useful insects such as honey bees and bumblebees and natural enemies such as aphelinids, Aphidiinae, tachina flies, Orius spp., Phytoseiidae spp. etc.).

The compounds of the present invention may be used in any dosage form such as a soluble concentrate, an emulsifiable concentrate, a wettable powder, a water soluble powder, a water dispersible granule, a water soluble granule, a suspension concentrate, a concentrated emulsion, a suspoemulsion, a microemulsion, a dustable powder, a granule, a tablet or an emulsifiable gel usually after mixed with an appropriate solid carrier or a liquid carrier, and if necessary, with a surfactant, a penetrant, a spreader, thickener, an anti-freezing agent, a binder, an anti-caking agent, a disintegrant, an antifoaming agent, a preservative, a stabilizer or the like. A formulation in an arbitrary dosage form may be sealed in water-soluble packaging such as a water-soluble capsule or a water-soluble film, for labor saving or improved safety.

As solid carriers, natural minerals such as quartz, calcite, meerschaum, dolomite, chalk, kaolinite, pyrophyllite, sericite, halloysite, methahalloysite, kibushi clay, gairome clay, pottery stone, zeeklite, allophone, Shirasu, mica, talc, bentonite, activated clay, pumice, attapulgite, zeolite and diatomaceous earth, calcined natural minerals such as calcined clay, pearlite, Shirasu-balloons, vermiculite, attapulgus clay and calcined diatomaceous earth, inorganic salts suchas magnesium carbonate, calcium carbonate, sodium carbonate, sodium hydrogen carbonate, ammonium sulfate, sodium sulfate, magnesium sulfate, diammonium hydrogen phosphate, ammonium dihydrogen phosphate and potassium chloride, saccharides suchas glucose, fructose, sucrose and lactose, polysaccharides such as starch, cellulose powder and dextrin, organic substances such as urea, urea derivatives, benzoic acid and benzoic acid salts, plants such as wood flour, powdered cork, corncob, walnut shell and tobacco stems, fly ash, white carbon (such as hydrated synthetic silica, anhydrous synthetic silica and hydrous synthetic silicate), fertilizers and the like may be mentioned.

As liquid carriers, aromatic hydrocarbons such as xylene, alkyl (C₉ or C₁₀ etc.) benzene, phenylxylylethane and alkyl (C₁ or C₃ etc.) naphthalene, aliphatic hydrocarbons such as machine oil, normal paraffin, isoparaffin and naphthene, mixtures of aromatic hydrocarbons and aliphatic hydrocarbons such as kerosene, alcohols such as ethanol, isopropanol, cyclohexanol, phenoxyethanol and benzyl alcohol, polyhydric alcohols such as ethylene glycol, propylene glycol, diethylene glycol, hexylene glycol, polyethylene glycol and polypropylene glycol, ethers such as propyl cellosolve, butyl cellosolve, phenyl cellosolve, propylene glycol monomethyl ether, propylene glycol monoethyl ether, propylene glycol monopropyl ether, propylene glycol monobutyl ether and propylene glycol monophenyl ether, ketones such as acetophenone, cyclohexanone and γ-butyrolactone, esters such as fatty acid methyl esters, dialkyl succinates, dialkyl glutamate, dialkyl adipates and dialkyl phthalates, acid amides such as N-alkyl (C₁, C₈ or C₁₂ etc.) pyrrolidone, fats and oils such as soybean oil, linseed oil, rapeseed oil, coconut oil, cottonseed oil and castor oil, dimethyl sulfoxide, water and the like may be mentioned.

These solid and liquid carriers may be used alone or in combinations of two or more.

As surfactants, nonionic surfactants such as polyoxyethylene alkyl ether, polyoxyethylene alkyl(mono or di)phenyl ether, polyoxyethylene(mono, di or tri)styrylphenyl ether, polyoxyethylenepolyoxypropylene block copolymers, polyoxyethylene fatty acid (mono or di)ester, sorbitan fatty acid ester, polyoxyethylene sorbitan fatty acid ester, ethylene oxide adducts of castor oil, acetylene glycol, acetylene alcohol, ethylene oxide adducts of acetylene glycol, ethylene oxide adducts of acetylene alcohol and alkyl glycosides, anionic surfactants such as alkyl sulfate salts, alkylbenzenesulfonic acid salts, lignin sulfonate, alkylsulfosuccinic acid salts, naphthalenesulfonic acid salts, alkylnaphthalenesulfonic acid salts, salts of naphthalenesulfonic acid-formalin condensates, salts of alkylnaphthalenesulfonic acid-formalin condensates, polyoxyethylene alkyl ether sulfate or phosphate salts, polyoxyethylene(mono or di)alkylphenyl ether sulfate or phosphate salts, polyoxyethylene(mono, di or tri)styrylphenyl ether sulfate or phosphate salts, polycarboxylic acid salts (such as polyacrylates, polymaleates and copolymers of maleic acid and an olefin) and polystyrenesulfonic acid salts, cationic surfactants such as alkylamine salts and alkyl quaternary ammonium salts, amphoteric surfactants such as amino acid types and betaine types, silicone surfactants and fluorine surfactants may be mentioned.

The amount of these surfactants is usually preferred to be from 0.05 to 20 parts by weight per 100 parts by weight of the agent of the present invention, though there is no particular restrictions. These surfactants may be used alone or in combination of two or more.

The suitable application dose of compounds of the present invention is generally about from 0.005 to 50 kg per hectare (ha) in terms of the active ingredient, though it varies depending on the application site, the application season, the application method and the cultivated crop.

When the compounds of the present invention are used to control external or internal parasites in and on mammals and birds as farm animals and pet aminals, the compounds of the present invention may be administered in an effective amount together with pharmaceutically acceptable additives orally, parenterally by injection (intramuscular, subcutaneously, intravenously or intraperitoneally); percutaneously by dipping, spraying, bathing, washing, pouring-on and spotting-on and dusting, or intranasally. The compounds of the present invention may be administered through molded articles such as chips, plates, bands, collars, ear marks, limb bands and ID tags. The compounds of the present invention are administered in an arbitrary dosage form suitable for the administration route.

The dosage form may be a solid preparation such as a dust, a granule, a wettable powder, a pellet, a tablet, a ball, a capsule and an molded article containing an active ingredient, a liquid preparation such as an injection fluid, an oral liquid, a liquid preparation applied to the skin or coelom, a pour-on preparation, a spot-on preparation, a flowable, an emulsion, and a semisolid preparation such as an ointment and a gel.

A solid preparation may generally be used by oral administration or by percutaneous or environmental application after dilution with water or the like. A solid preparation can be prepared by mixing an active ingredient with an appropriate vehicle, and with an adjuvant if necessary, and formulating the mixture into a desired dosage form. As the vehicle, an inorganic vehicle such as a carbonate, a hydrogen carbonate, a phosphate, aluminum oxide, silica or clay or an organic vehicle such as a saccharide, cellulose, cereal flour or starch.

An injection fluid may be administered intravenously, intramuscularly or subcutaneously. An injection fluid can be prepared by dissolving an active ingredient in an appropriate solvent and, if necessary, adding additives such as a solubilizer, an acid, a base, a buffering salt, an antioxidant and a protectant. As appropriate solvents, water, ethanol, butanol, benzyl alcohol, glycerine, propylene glycol, polyethylene glycol, N-methylpyrrolidone and mixtures thereof, physiologically acceptable vegetable oils and synthetic oils suitable for injection may be mentioned. As solubilizers, polyvinylpyrrolidone, polyoxyethylated castor oil, polyoxyethylated sorbitan ester and the like may be mentioned. As protectants, benzyl alcohol, trichlorobutanol, p-hydroxybenzoic acid esters, n-butanol and the like may be mentioned.

An oral liquid may be administered directly or after dilution and can be prepared in the same manner as an injection fluid.

A flowable, an emulsion or the like may be administered directly or after dilution percutaneously or by environmental application.

A liquid preparation applied to the skin is administered by dripping, spreading, rubbing, spraying, sprinkling or dipping (soaking, bathing or washing) and can be prepared in the same manner as an injection fluid.

A pour-on preparation and a spot-on preparation are dripped or sprayed to a limited area of the skin so that they permeate through the skin and act systemically. A pour-on preparation and a spot-on preparation can be prepared by dissolving, suspending or emulsifying an active ingredient in an skin-friendly solvent or solvent mixture. If necessary, additives such as a surfactant, a colorant, an absorbefacient, an antioxidant, a light stabilizer and an adhesive.

As appropriate solvents, water, alkanol, glycol, polyethylene glycol, polypropylene glycol, glycerine, benzyl alcohol, phenylethanol, phenoxyethanol, ethyl acetate, butyl acetate, benzyl benzoate, dipropylene glycol monomethyl ether, diethylene glycol monobutyl ether, acetone, methyl ethyl ketone, aromatic and/or aliphatic hydrocarbons, vegetable or synthetic oils, DMF, liquid paraffin, light liquid paraffin, silicone, dimethylacetamide, N-methylpyrrolidone or 2,2-dimethyl-4-oxymethylene-1,3-dioxolane may be mentioned. As absorbefacients, DMSO, isopropyl myristate, pelargonic acid dipropylene glycol, silicone oil, fatty acid esters, triglycerides and aliphatic alcohols may be mentioned. As antioxidants, sulfites, metabisulfites, ascorbic acid, butylhydroxytoluene, butylhydroxyanisole and tocopherol may be mentioned.

An emulsion may be administered orally, percutaneously or by injection. An emulsion can be prepared by dissolving an active ingredient in a hydrophobic phase or a hydrophilic phase and homogenizing the resulting solution with another liquid phase together with an appropriate emulsifier, and further with additives such as a colorant, if necessary an absorbefacient, a protectant, an antioxidant, a light screen and a thickener.

As hydrophobic phases (oils), paraffin oil, silicone oil, sesame oil, almond oil, castor oil, synthetic triglycerides, ethyl stearate, di-n-butyryl adipate, hexyl laurate, pelargonic acid dipropylene glycol, esters of branched short-chain fatty acids with C16-C18 saturated fatty acids, isopropyl myristate, isopropyl palmitate, esters of C12-C18 saturated alcohols with caprylic/capric acid, isopropyl stearate, oleyl oleate, decyl oleate, ethyl oleate, ethyl lactate, fatty acid ester waxes, dibutyl phthalate, diisopropyl adipate, isotridecyl alcohol, 2-octyldodecanol, cetylstearyl alcohol and oleyl alcohol may be mentioned.

As hydrophilic phases, water, propylene glycol, glycerine and sorbitol may be mentioned.

As emulsifiers, nonionic surfactants such as polyoxyethylated castor oil, polyoxyethylated sorbitan monoolefinic acid, sorbitan monostearate, glycerine monostearate, polyoxyethyl stearate and alkyl phenol polyglycol ether; amphoteric surfactants such as disodium N-lauryl-β-iminodipropionate and lecithin; and anionic surfactants such as sodium lauryl sulfate, aliphatic alcohol sulfate ether, mono/dialkylpolyglycol orthophosphate monoethanolamine salt may be mentioned.

As other additives, carboxymethylcellulose, methylcellulose, polyacrylate, alginate, gelatin, gum arabic, polyvinylpyrrolidone, polyvinyl alcohol, methyl vinyl ether, maleic anhydride copolymers, polyethylene glycol, waxes and colloidal silica may be mentioned.

A semisolid preparation is administered by applying or spreading onto the skin or introducing into the coelom. A gel can be prepared by adding a thickener to a solution prepared in the same manner as an injection fluid sufficiently to give a transparent viscous substance like an ointment.

Next, formulation examples of preparations using the compounds of the present invention are given below. However, formulations of the present invention are by no means restricted thereto. In the following Formulation Examples, “parts” means parts by weight.

### [Wettable Powder]

| | |
|---|---|
| Compound of the present invention | 0.1 to 80 parts |
| Solid carrier | 5 to 98.9 parts |
| Surfactant | 1 to 10 parts |
| Others | 0 to 5 parts |

As the others, an anti-caking agent, a stabilizer and the like may be mentioned.

### [Emulsion]

| | |
|---|---|
| Compound of the present invention | 0.1 to 30 parts |
| Liquid carrier | 45 to 95 parts |
| Surfactant | 4.9 to 15 parts |
| Others | 0 to 10 parts |

As the others, a spreader, a stabilizer and the like may be mentioned.

### [Suspension]

| | |
|---|---|
| Compound of the present invention | 0.1 to 70 parts |
| Liquid carrier | 15 to 98.89 parts |
| Surfactant | 1 to 12 parts |
| Others | 0.01 to 30 parts |

As the others, an anti-freezing agent, a thickener and the like may be mentioned.

### [Water Soluble Granule]

| | |
|---|---|
| Compound of the present invention | 0.1 to 90 parts |
| Solid carrier | 0 to 98.9 parts |
| Surfactant | 1 to 20 parts |
| Others | 0 to 10 parts |

As the others, a binder, a stabilizer and the like may be mentioned.

### [Soluble Concentrate]

| | |
|---|---|
| Compound of the present invention | 0.01 to 70 parts |
| Liquid carrier | 20 to 99.99 parts |
| Others | 0 to 10 parts |

As the others, an anti-freezing agent, a spreader and the like may be mentioned.

### [Granule]

| | |
|---|---|
| Compound of the present invention | 0.01 to 80 parts |
| Solid carrier | 10 to 99.99 parts |
| Others | 0 to 10 parts |

As the others, a bind, a stabilizer and the like may be mentioned.

### [Dust]

| | | |
|---|---|---|
| Compound of the present invention | 0.01 to 30 | parts |
| Solid carrier | 65 to 99.99 | parts |
| Others | 0 to 5 | parts |

As the others, an anti-drift agent, a stabilizer and the like may be mentioned.

Next, more specific examples of preparations containing compounds of the present invention as an active ingredient are by no means restricted thereto.

In the following Formulation Examples, “parts” means parts by weight.

### Formulation Example 1

### Wettable Powder

| | |
|---|---|
| Compound No. 1-001 of the present invention | 20 parts |
| Pyrophyllite | 74 parts |
| Sorpol 5039 | 4 parts |
| (trade name for a mixture of a nonionic surfactant and an anionic | |
| surfactant: manufactured by TOHO Chemical Industry | |
| Col., Ltd.) | |
| CARPLEX #80D | 2 parts |
| (hydrous synthetic silicic acid: trade name manufactured by | |
| Shionogi & Co., Ltd.) | |

The above ingredients are mixed and pulverized homogenously to obtain a wettable powder.

### Formulation Example 2

### Emulsifiable Concentrate

| | |
|---|---|
| Compound No. 1-001 of the present invention | 5 parts |
| xylene | 75 parts |
| N-methylpyrrolidone | 15 parts |
| Sorpol 2680 | 5 parts |
| (trade name for a mixture of a nonionic surfactant and an anionic | |
| surfactant: manufactured by TOHO Chemical | |
| Industry Co., Ltd.) | |

The above ingredients are mixed homogenously to obtain an emulsifiable concentrate.

### Formulation Example 3

### Suspension Concentrate

| | | |
|---|---|---|
| Compound No. 1-001 of the present invention | 25 | parts |
| AGRISOL S-710 | 10 | parts |
| (trade name for a nonionic surfactant: manufactured by Kao | | |
| Corporation) | | |
| Lunox 1000C | 0.5 | part |
| (trade name for an anionic surfactant: manufactured by | | |
| TOHO Chemical Industry Co., Ltd.) | | |
| Xanthan gum | 0.2 | part |
| Water | 64.3 | parts |

The above ingredients are mixed homogenously and wet-pulverized to obtain a suspension concentration.

### Formulation Example 4

### Water Soluble Granule

| | |
|---|---|
| Compound No. 1-001 of the present invention | 75 parts |
| HITENOL NE-15 | 5 parts |
| (trade name for an anionic surfactant: manufactured by Dai-ichi | |
| Kogyo Seiyaku Co., Ltd.) | |
| VANILLEX N | 10 parts |
| (trade name for an anionic surfactant: manufactured by Nippon | |
| Paper Industries Co., LTD.) | |
| CARPLEX #80D | 10 parts |
| (trade name for hydrous synthetic silicic acid: manufactured by | |
| Shionogi & Co., Ltd.) | |

The above ingredients are mixed and pulverized homogenously, then kneaded with a small amount of water, granulated through an extrusion granulator and dried to obtain a water soluble granule.

### Formulation Example 5

### Granule

| | |
|---|---|
| Compound No. 1-001 of the present invention | 5 parts |
| Bentonite | 50 parts |
| Talc | 45 parts |

The above ingredients are mixed and pulverized homogenously, then kneaded with a small amount of water, granulated through an extrusion granulator and dried to obtain a granule.

### Formulation Example 6

### Dust

| | | |
|---|---|---|
| Compound No. 1-001 of the present invention | 3 | parts |
| CARPLEX #80D | 0.5 | part |
| (trade name for a hydrous synthetic silicic acid: manufactured | | |
| by Shionogi & Co., Ltd.) | | |
| Kaolinite | 95 | parts |
| Diisopropyl phosphate | 1.5 | parts |

The above ingredients are mixed and pulverized homogeneously to obtain a dust.

It is applied after diluted with water by a factor of from 1 to 10000 or directly without dilution.

### Formulation Example 7

### Wettable Powder Preparation

| | | |
|---|---|---|
| Compound No. 1-001 of the present invention | 25 | parts |
| Sodium diisobutylnaphthalenesulfonate | 1 | part |
| Calcium n-dodecylbenzenesulfonate | 10 | parts |
| Alkyl aryl polyglycol ether | 12 | parts |
| Naphthalenesulfonic acid-formalin condensate sodium salt | 3 | parts |
| Silicone emulsion | 1 | part |
| Silicon dioxide | 3 | parts |
| Kaolin | 45 | parts |

### Formulation Example 8

### Water-Soluble Concentrate Preparation

| | |
|---|---|
| Compound No. 1-001 of the present invention | 20 parts |
| Polyoxyethylenelauryl ether | 3 parts |
| Sodium dioctylsulfosuccinate | 3.5 parts |
| Dimethyl sulfoxide | 37 parts |
| 2-Propanol | 36.5 parts |

### Formulation Example 9

### Liquid Preparation for Spraying

| | |
|---|---|
| Compound No. 1-001 of the present invention | 2 parts |
| Dimethyl sulfoxide | 10 parts |
| 2-Propanol | 35 parts |
| Acetone | 53 parts |

### Formulation Example 10

### Liquid Preparation for Percutaneous Administration

| | |
|---|---|
| Compound No. 1-001 of the present invention | 5 parts |
| Hexylene glycol | 50 parts |
| Isopropanol | 45 parts |

### Formulation Example 11

### Liquid Preparation for Percutaneous Administration

| | |
|---|---|
| Compound No. 1-001 of the present invention | 5 parts |
| Propylene glycol monomethyl ether | 50 parts |
| Dipropylene glycol | 45 parts |

### Formulation Example 12

### Liquid Preparation for Percutaneous Administration (by Dripping)

| | |
|---|---|
| Compound No. 1-001 of the present invention | 2 parts |
| Light liquid paraffin | 98 parts |

### Formulation Example 13

### Liquid Preparation for Percutaneous Administration (by Dripping)

| | | |
|---|---|---|
| Compound No. 1-001 of the present invention | 2 | parts |
| Light liquid paraffin | 58 | parts |
| Olive oil | 30 | parts |
| ODO-H | 9 | parts |
| Shin-etsu silicone | 1 | part |

For use as agrochemicals, if necessary, the compounds of the present invention may be mixed with other herbicides, various insecticides, miticides, nematocides, plant growth regulators, synersists, fertilizers, soil conditioners and the like at the time of formulation or application.

Particularly, the combined use with other agrochemicals or plant hormones is expected to reduce the application cost by enabling control at lower doses and lead to a broader insecticidal spectrum and higher insecticidal effect due to the synergistic effect of the other agrochemicals. In such cases, they may be combined with a plurality of known agrochemicals. The agrochemicals to be used in combination with the compounds of the present invention include, for example, the compounds disclosed in The Pesticide Manual, 15th edition, 2009, having the generic names recited below, but are not necessarily restricted thereto.

Fungicide: such as acibenzolar-S-methyl, acylaminobenzamide, acypetacs, aldimorph, ametoctradin, amisulbrom, amobam, ampropylos, anilazine, azaconazole, azithiram, azoxystrobin, barium polysulfide, benalaxyl, benalaxyl-M, benodanil, benomyl, benquinox, bentaluron, benthiavalicarb, benthiazole, benzamacril, benzamorf, bethoxazine, binapacryl, biphenyl, bitertanol, blasticidin-S, bixafen, bordeaux mixture, boscalid, bromoconazole, bupirimate, buthiobate, calcium polysulfide, calcium polysulfide, captafol, captan, carpropamid, carbamorph, carbendazim, carboxin, carvone, cheshunt mixture, chinomethionat, chlobenthiazone, chloraniformethane, chloranil, chlorfenazol, chloroneb, chloropicrin, chlorothalonil, chlorquinox, chlozolinate, climbazole, clotrimazole, copper acetate, copper carbonate, basic, copper hydroxide, copper naphthenate, copper oleate, copper oxychloride, sulfate, copper sulfate, basic, copper zinc chromate, cufraneb, cuprobam, cyazofamid, cyclafuramid, cycloheximide, cyflufenamid, cymoxanil, cypendazole, cyproconazol, cyprodinil, cyprofuram, dazomet, debacarb, decafentin, dehydroacetic acid, dichlofluanid, dichlone, dichlorophen, dichlozoline, diclobutrazol, diclocymet, diclomedine and dicloran.

Fungicide (continued): such as diethofencarb, difenoconazole, diflumetorim, dimethirimol, dimethomorph, dimoxystrobin, diniconazole, diniconazole-M, dinobuton, dinocap, dinocap-4, dinocap-6, dinocton, dinosulfon, dinoterbon, diphenylamine, dipyrithione, ditalimfos, dithianon, dodemorph, dodine, drazoxolon, edifenphos, epoxiconazole, etaconazole, ethaboxam, etem, ethirimol, ethoxyquin, etridiazole, famoxadone, fenarimol, febuconazole, fenamidone, fenaminosulf, fenapanil, fendazosulam, fenfuram), fenhexamid, fenitropan, fenoxanil, fenpiclonil, fenpropidin, fenpyrazamine, fenpropimorph, fentin, ferbam, ferimzone, fluazinam, fludioxonil, flumetover, flumorph, fluopicolide, fluopyram, fluoroimide, fluotrimazole, fluoxastrobin, fluquinconazole, flusilazole, flusulfamide, flutianil, flutolanil, flutriafol, fluxapyroxad, folpe), fosetyl-aluminium, fuberidazole, furalaxyl, furametpyr, furcarbanil, furconazole, furconazole-cis, furmecyclox, furphanate, glyodin, griseofulvin, guazatine, halacrinate, hexachlorobenzene, hexaconazole, hexylthiofos, 8-hydroxyquinoline sulfate, hymexazol, imazalil, imibenconazole, iminoctadine, ipconazole, iprobenfos, iprodione, iprovalicarb, isoprothiolane and isovaledione.

Fungicide (continued): such as kasugamycin, kresoxim-methyl, mancopper, mancozeb, mandipropamid, maneb, mebenil, mecarbinzid, mepanipyrim, mepronil, metalaxyl, metalaxyl-M, metam, metazoxolon, metconazole, methasulfocarb, methfuroxam, methyl isothiocyanate, metiram, metominostrobin, metrafenone, metsulfovax, milneb, myclobutanil, myclozolin, nabam, natamycin, nickel bis(dimethyldithiocarbamate), nitrostyrene, nitrothal-isopropyl, nuarimol, OCH, octhilinone, ofurace, orysastrobin, oxadixyl, oxine copper, oxycarboxin, (oxpoconazole fumarate, pefurzoate, penconazole, penflufen, pencycuron, penthiopyrad, o-phenylphenol, phosdiphen, phthalide, picoxystrobin, piperalin, polycarbamate, polyoxins, polyoxorim, potassium azide, potassium hydrogen carbonate, proquinazid, probenazole, prochloraz, procymidone, propamocarb hydrochloride, propiconazole, propineb, prothiocarb, prothioconazole, pyracarbolid, pyraclostrobin, pyrazophos, pyridinitril, pyrifenox, pyrimethanil, pyriofenone, pyroquilon, pyroxychlor, pyroxyfur, quinomethionate, quinoxyfen, quintozene, quinacetol-sulfate, quinazamid, quinconazole and rabenzazole.

Fungicide (continued): such as sodium azide, sodium hydrogen carbonate, sodium hypochlorite, sulfur, spiroxamine, salycylanilide, silthiofam, simeconazole, tebuconazole, tecnazene, tecoram, tetraconazole, thiabendazole, thiadifluor, thicyofen, thifluzamide, thiochlorfenphim, thiophanate, thiophanate-methyl, thioquinox, thiram, tiadinil, tioxymid, tolclofos-methyl, tolylfluanid, triadimefon, toriadimenol, triamiphos, triarimol, triazoxide, triazbutil, tributyltin oxide, trichiamide, tricyclazole, tridemorph, trifloxystrobin, triflumizole, triforine, triticonazole, validamycin, valifenalate, vinclozolin, zarilamide, zinc sulfate, zineb, ziram, zoxamide and shiitake mushroom mycelium extracts.

Bacteriocides: such as benzalkonium chloride, bithionol, bronopol, cresol, formaldehyde, nitrapyrin, oxolinic acid, oxyterracycline, streptomycin and tecloftalam.

Nematocides: such as aldoxycarb, cadusafos, DBCP, dichlofenthion, DSP, ethoprophos, fenamiphos, fensulfothion, fluensulfone, fosthiazate, fosthietan, imicyafos, isamidofos, isazofos, oxamyl and thionazin.

Miticides: such as acequinocyl, acrinathrin, amitraz, BCI-033 (experimental name), bifenazate, bromopropylate, chinomethionat, chlorobezilate, clofentezine, cyenopyrafen, cyflumetofen, cyhexatine, dicofol, dienochlor, DNOC, etoxazole, fenazaquin, fenbutatin oxide, fenothiocarb, fenpropathrin, fenpyroximate, fluacrypyrim, halfenprox, hexythiazox, milbemectin, propargite, pyridaben, pyrimidifen, S-1870 (experimental name), spirodiclofen, spyromesifen, NNI-0711 (experimental name), CL900167 (experimental name) and tebufenpyrad.

Insecticides: such as abamectin, acephate, acetamipirid, alanycarb, aldicarb, allethrin, azamethiphos, azinphos-methyl, bacillus thuringiensis, bendiocarb, benfluthrin, benfuracarb, bensultap, bifenthrin, bioallethrin, bioresmethrin, bistrifluoron, buprofezin, butocarboxim, carbaryl, carbofuran, carbosulfan, cartap, chlorantraniliprole, chlorethxyfos, chlorfenapyr, chlorfenvinphos, chlorfluazuron, chlormephos, chlorpyrifos, chlorpyrifos-methyl, chromafenozide, clothianidin, cyantraniliprole, cycloprothrin, cyflumetofen, cyfluthrin, beta-cyfluthrin, cyhalothrin, lambda-cyhalothrin, cypermethrin, alpha-cypermethrin, beta-cypermethrin, zeta-cypermethrin, cyromazine, deltamethrin, diacloden, diafenthiuron, diazinon, dichlorvos, diflubenzuron, dimethylvinphos, dinotefuran, diofenolan, disulfoton, dimethoate, emamectin-benzoate, empenthrin, endosulfan, alpha-endosulfan, EPN, esfenvalerate, ethiofencarb, ethiprole, etofenprox, etrimfos, fenitrothion, fenobucarb, fenoxycarb, fenpropathrin, fenthion, fenvalerate, fipronil, flonicamid, flubendiamide, flucythrinate, flufenerim, flufenoxuron, flufenprox, flumethrin, fluvalinate, tau-fluvalinate, fonophos, formetanate, formothion, furathiocarb, flufiprole, flupyradifurone and flometoquin.

Insecticides (continued): such as halofenozide, hexaflumuron, hydramethylnon, imidacloprid, isofenphos, indoxacarb, isoprocarb, isoxathion, lepimectin, lufenuron, malathion, meperfluthrin, metaflumizone, metaldehyde, methamidophos, methidathion, methacrifos, metaflumizone, metalcarb, methomyl, methoprene, methoxychlor, methoxyfenozide, methyl bromide, monocrotophos, muscalure, nitenpyram, novaluron, noviflumuron, omethoate, oxamyl, oxydemeton-methyl, oxydeprofos, parathion, parathion-methyl, pentachlorophenol (PCP), permethrin, phenothrin, phenthoate, phoxim, phorate, phosalone, phosmet, phosphamidon, pirimicarb, pirimiphos-methyl, profenofos, prothiofos, propaphos, protrifenbute, pymetrozine, pyraclofos, pyrethrins, pyridalyl, pyrifluquinazon, pyriprole, pyrafluprole, pyriproxyfen, resmethrin, rotenone, SI-0405 (experimental name), sulprofos, silafluofen, spinetoram, spinosad, spirotetramat, sulfoxaflor, sulfotep, SYJ-159 (experimental name), tebfenozide, teflubenzuron, tefluthorin, terbufos, tetrachlorvinphos, tetramethrin, d-tetramethrin, tetramethylfluthrin, thiacloprid, thiocyclam, thiodicarb, thiamethoxam, thiofanox, thiometon, tolfenpyrad, tralomethrin, trichlorfon, triazuron, triflumuron, vamidothion and ME-5343 (experimental name).

### EXAMPLES

Now, the present invention will be described in further detail by referring to the following specific Examples of syntheses of and tests on the compounds of the present invention. However, the present invention is by no means restricted thereto.

For the preparative medium pressure liquid chromatography, the preparative medium pressure chromatograph; YFLC-Wprep manufactured by Yamazen Science, Inc., (flow rate 18 ml/min, 40-μm silica gel column) was used.

### SYNTHETIC EXAMPLES

### Synthetic Example 1

### Preparation of tert-butyl 3-methyl-1-(pyridin-3-yl)-1H-pyrazol-4-ylcarbamate (Compound No. 1-011 of the present invention)

### Step 1

### Preparation of ethyl 3-methyl-1-(pyridin-3-yl)-1H-pyrazole-4-carboxylate

3.0 g of ethyl 3-methyl-1H-pyrazole-4-carboxylate in 10 ml of N,N-dimethylformamide was mixed with 5.3 g of 3-iodopyridine, 990 mg of copper (I) iodide and 17.0 g of cesium carbonate successively. After the atmosphere in the reaction vessel was replaced by nitrogen gas, the mixture was stirred at 120° C. for 4 hours. After the reaction, the reaction mixture was mixed with 100 ml of water and extracted with ethyl acetate (150 ml×1). The resulting organic layer was dried over anhydrous sodium sulfate, and the solvent was evaporated under reduced pressure. The resulting residue was purified by silica gel column chromatography using n-hexane-ethyl acetate {1:1 (volume ratio, hereinafter the same applies)} as the eluent to give 2.4 g of the desired product as white crystals.

¹H NMR (CDCl₃, Me₄Si, 300 MHz) δ8.98 (d, J=2.4 Hz, 1H), 8.58 (d, J=3.9 Hz, 1H), 8.38 (s, 1H), 8.00-8.10 (m, 1H), 7.42 (dd, J=4.8, 8.1 Hz, 1H), 4.34 (q, J=7.2 Hz, 2H), 2.57 (s, 3H), 1.38 (t, J=7.2 Hz, 3H), (no detectable proton peaks for CO₂H)

### Step 2

### Preparation of 3-methyl-1-(pyridin-3-yl)-1H-pyrazole-4-carboxylic acid

3.9 g of ethyl 3-methyl-1-(pyridin-3-yl)-1H-pyrazole-4-carboxylate was dissolved in 20 mL of ethanol, and 1.9 g of potassium hydroxide and 10 mL of water were added successively. After the addition, the reaction mixture was stirred at 50° C. for 1 hour. After the reaction, the solvent was evaporated under reduced pressure, and the resulting residue was neutralized with aqueous hydrochloric acid and extracted with ethyl acetate (50 ml×2). The resulting organic layer was dried over anhydrous sodium sulfate, and the solvent was evaporated under reduced pressure to give 2.1 g of the desired product as a white solid.

¹H NMR (DMSO-d₆, Me₄Si, 300 MHz) δ9.03 (m, 1H), 8.71 (s, 1H), 8.46 (d, J=2.1 Hz, 1H), 8.18 (d, J=8.4 Hz, 1H), 7.50 (dd, J=8.4 Hz, 4.8 Hz, 1H), 2.40 (s, 3H)

m. p.; 200-210° C.

### Step 3

### Preparation of tert-butyl 3-methyl-1-(pyridin-3-yl)-1H-pyrazol-4-ylcarbamate

To 4.98 g of 3-methyl-1-(pyridin-3-yl)-1H-pyrazole-4-carboxylic acid in 50 ml of 2-methyl-2-propanol, 7.44 g of triethylamine and 8.78 g of diphenylphosphoryl azide were added at room temperature successively. After the addition, the reaction mixture was refluxed with 2-methyl-2-propanol for 4 hours with stirring. After the reaction, the reaction mixture was allowed to cool to room temperature and poured into ice-water and extracted with 50 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (1:1) as the eluent to give 3.67 g of the desired product as a white solid.

¹H NMR (CDCl₃, Me₄Si, 300 MHz) δ8.94 (d, J=1.2 Hz, 1H), 8.46 (dd, J=4.5, 2.7 Hz, 1H), 8.25 (brs, 1H), 7.95 (ddd, J=8.4, 2.7, 1.2 Hz, 1H), 7.33 (dd, J=8.4, 4.5 Hz, 1H), 6.18 (brs, 1H), 2.28 (s, 3H), 1.52 (s, 9H)

### Synthetic Example 2

### Preparation of tert-butyl methyl{3-methyl-1-(pyridin-3-yl)-1H-pyrazol-4-yl}carbamate (Compound No. 1-012 of the present invention)

To 83 mg of 60 wt % sodium hydride (dispersed in mineral oil) in 5 ml of N,N-dimethylformamide,
500 mg of tert-butyl 3-methyl-1-(pyridin-3-yl)-1H-pyrazol-4-ylcarbamate was added under cooling with ice. After the addition, the reaction mixture was allowed to warm to room temperature and stirred at room temperature for 30 minutes. After the stirring, the reaction mixture was mixed with 284 mg of methyl iodide and stirred at room temperature for other 30 minutes. After the reaction, the reaction mixture poured into ice-water and extracted with 10 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (2:1) as the eluent to give 0.44 g of the desired product as a white solid.

¹H NMR (CDCl₃, Me₄Si, 300 MHz) δ8.89 (d, J=2.7 Hz, 1H), 8.49 (dd, J=4.8, 1.2 Hz, 1H), 8.03-7.95 (m, 1H), 7.82 (brs, 1H), 7.36 (dd, J=8.1, 4.8 Hz, 1H), 3.19 (s, 3H), 2.26 (s, 3H), 1.44 (brs, 9H)

### Synthetic Example 3

### Preparation of N-methyl-N-{3-methyl-1-(pyridin-3-yl)-1H-pyrazol-4-yl}-3-(methylthio)propanamide (Compound No. 1-018 of the present invention)

### Step 1

### Preparation of N,3-dimethyl-1-(pyridin-3-yl)-1H-pyrazole-4-amine dihydrochloride (Compound No. A-001)

0.35 g of tert-butyl methyl{3-methyl-1-(pyridin-3-yl)-1H-pyrazol-4-yl}carbamate in 5 ml of 1,4-dioxane was mixed with 1.2 ml of about 4 M hydrogen chloride in 1,4-dioxane and stirred at room temperature for 2 days. After the reaction, the impurities in the reaction mixture were filtered off under reduced pressure, and the filtrate was evaporated under reduced pressure to remove the solvent to give 0.34 g of the desired product as a white solid.

m. p.; 174-179° C.

### Step 2

### Preparation of N-methyl-N-{3-methyl-1-(pyridin-3-yl)-1H-pyrazol-4-yl}-3-(methylthio)propanamide

150 mg of N,3-dimethyl-1-(pyridin-3-yl)-1H-pyrazole-4-amine dihydrochloride in 5 ml of dichloromethane was mixed with 174 mg of triethylamine, 103 mg of 3-(methylthio)propanoic acid, 165 mg of 1-ethyl-3-(3-dimethylaminopropyl)carbodiimide hydrochloride and 10 mg of 4-dimethylaminopyridine successively and stirred at room temperature for 12 hours. After the reaction, the reaction mixture was mixed with 10 ml of water and extracted with 10 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (1:2) as the eluent t to give 78 mg of the desired product as a yellow oil.

¹H NMR (CDCl₃, Me₄Si, 300 MHz) δ8.93 (d, J=2.7 Hz, 1H), 8.56 (dd, J=4.8, 1.2 Hz, 1H), 8.01 (ddd, J=8.4, 2.7, 1.2 Hz, 1H), 7.90 (s, 1H), 7.41 (dd, J=8.4, 4.8 Hz, 1H), 3.22 (s, 3H), 2.77 (t, J=7.2 Hz, 2H), 2.43 (t, J=7.2 Hz, 2H), 2.27 (s, 3H), 2.04 (s, 3H)

### Synthetic Example 4

### Preparation of 2,5-dibromo-N-{1-(pyridin-3-yl)-1H-pyrazol-4-yl}pentanamide (Compound No. 1-025 of the present invention)

To 1.5 g of 1-(pyridin-3-yl)-1H-pyrazole-4-amine dihydrochloride in 20 ml of dichloromethane, 2.3 g of triethylamine and 2.5 g of 2,5-dibromovaleryl bromide were added under cooling with ice successively. After the addition, the reaction mixture was stirred at room temperature for 3 hours. After the reaction, the reaction mixture was mixed with 20 ml of water and extracted with 20 ml of chloroform. The resulting organic layer was dried over anhydrous sodium sulfate, and the solvent was evaporated under reduced pressure. The resulting residue was purified by preparative medium pressure liquid chromatography using n-hexane-ethyl acetate (with a gradient from 9:1 to 1:3) as the eluent to give 1.7 g of the desired product as a white solid.

¹H NMR (CDCl₃, Me₄Si, 300 MHz) δ9.01 (d, J=2.4 Hz, 1H), 8.56 (s, 1H), 8.56 (dd, J=4.8, 1.2 Hz, 1H), 8.14 (brs, 1H), 8.03 (ddd, J=8.1, 2.4, 1.2 Hz, 1H), 7.75 (s, 1H), 7.41 (dd, J=8.1, 4.8 Hz, 1H), 4.53 (dd, J=7.8, 4.8 Hz, 1H), 3.47 (t, J=6.3 Hz, 2H), 2.38-2.50 (m, 1H), 2.24-2.36 (m, 1H), 2.07-2.18 (m, 2H)

### Synthetic Example 5

### Preparation of 3-bromo-1-{1-(pyridin-3-yl)-1H-pyrazol-4-yl}piperidin-2-one (Compound No. 1-027 of the present invention)

To 1.54 g of 2,5-dibromo-N-{1-(pyridin-3-yl)-1H-pyrazol-4-yl}pentanamide in 30 ml of tetrahydrofuran, 0.29 g of 60 wt % sodium hydride (dispersed in mineral oil) was added under cooling with ice. After the addition, the reaction mixture was stirred at room temperature for 4 hours. After the reaction, the reaction mixture was mixed with saturated aqueous ammonium chloride and extracted with 40 ml of ethyl acetate. The resulting organic layer was dried over anhydrous sodium sulfate, and the solvent was evaporated under reduced pressure. The precipitated solid was washed with diisopropyl ether to give 1.11 g of the desired product as a pale yellow solid.

¹H NMR (CDCl₃, Me₄Si, 300 MHz) δ9.02 (d, J=2.7 Hz, 1H), 8.72 (s, 1H), 8.56 (dd, J=4.8, 1.5 Hz, 1H), 8.03 (ddd, J=8.4, 2.7, 1.5 Hz, 1H), 7.82 (s, 1H), 7.41 (dd, J=8.4, 4.8 Hz, 1H), 4.77 (dt, J=4.2, 1.2 Hz, 1H), 3.80-3.94 (m, 2H), 2.43-2.50 (m, 1H), 2.36-2.41 (m, 2H), 2.02-2.10 (m, 1H)

### Synthetic Example 6

### Preparation of 3-(methylthio)-1-{1-(pyridin-3-yl)-1H-pyrazol-4-yl}piperidin-2-one (Compound No. 1-028 of the present invention)

400 mg of 3-bromo-1-(1-(pyridin-3-yl)-1H-pyrazol-4-yl)piperidin-2-one in 2 ml of dimethyl sulfoxide was mixed with 105 mg of sodium methylmercaptan and stirred at room temperature for 5 hours. After the reaction, the reaction mixture was mixed with 5 ml of water and extracted with 10 ml of ethyl acetate. The resulting organic layer was dried over anhydrous sodium sulfate, and the solvent was evaporated under reduced pressure. The precipitated solid was washed with diisopropyl ether to give 304 mg of the desired product as a pale yellow solid.

¹H NMR (CDCl₃, Me₄Si, 300 MHz) δ9.02 (d, J=2.7 Hz, 1H), 8.67 (s, 1H), 8.55 (dd, J=4.8, 1.5 Hz, 1H), 8.04 (ddd, J=8.1, 2.7, 1.5 Hz, 1H), 7.81 (s, 1H), 7.40 (dd, J=8.1, 4.8 Hz, 1H), 3.71-3.88 (m, 2H), 3.53 (t, J=5.1 Hz, 1H), 2.38 (s, 3H), 2.20-2.32 (m, 2H), 2.02-2.10 (m, 2H)

### Synthetic Example 7

### Preparation of tert-butyl 3-chloro-1-(pyridin-3-yl)-1H-pyrazol-4-ylcarbamate (Compound No. 1-046 of the present invention)

### Step 1

### Preparation of ethyl 3-chloro-1H-pyrazole-4-carboxylate

9.71 g of ethyl 1-tert-butyl-3-chloro-1H-pyrazole-4-carboxylate was mixed with 30 ml of concentrated hydrochloric acid under cooling with ice and stirred overnight at room temperature. After the stirring, the reaction mixture was mixed with 10 ml of concentrated hydrochloric acid and stirred for one day and night. After the reaction, the reaction mixture was cooled to 0° C., neutralized with aqueous sodium hydroxide and extracted with 100 ml of ethyl acetate. The resulting organic layer was dried over anhydrous sodium sulfate, and the solvent was evaporated under reduced pressure. The resulting solid was washed with n-hexane to give 6.28 g of the desired product as a white solid.

¹H NMR (CDCl₃, Me₄Si, 300 MHz) δ11.2 (brs, 1H), 8.10 (s, 1H), 4.33 (q, J=7.2 Hz, 2H), 1.36 (t, J=7.2 Hz, 3H)

### Step 2

### Preparation of ethyl 3-chloro-1-(pyridin-3-yl)-1H-pyrazole-4-carboxylate

To 10.2 g of ethyl 3-chloro-1H-pyrazole-4-carboxylate in 150 ml of N,N-dimethylformamide, 15.6 g of 3-iodopyridine, 2.23 g of copper (I) iodide and 33.0 g of cesium carbonate were added successively. After the addition, the atmosphere in the reaction vessel was replaced by nitrogen gas, and the reaction mixture was stirred at 130° C. for 5 hours. After the reaction, the reaction mixture was allowed to cool to room temperature and mixed with 100 ml of 1 N aqueous sodium hydroxide. The impurities in the reaction mixture were filtered off through Celite under reduced pressure, and the filtrate was extracted with 100 ml of ethyl acetate. The resulting organic layer was washed with saturated aqueous sodium chloride and then dried over anhydrous sodium sulfate, and the solvent was evaporated under reduced pressure. The resulting residue was purified by preparative medium pressure liquid chromatography using n-hexane-ethyl acetate (with a gradient of 1:1) as the eluent to give 7.56 g of the desired product as a white solid.

m. p.; 90-93° C.

### Step 3

### Preparation of 3-chloro-1-(pyridin-3-yl)-1H-pyrazole-4-carboxylic acid

1.2 g of ethyl 3-chloro-1-(pyridin-3-yl)-1H-pyrazole-4-carboxylate was dissolved in 15 ml of ethanol, mixed with 0.6 g of potassium hydroxide and 10 ml of water successively and stirred at room temperature for 1 hour. After the reaction, the solvent was evaporated under reduced pressure. The resulting residue was acidified to pH 3 with 1 N aqueous hydrogen chloride, and the solvent was evaporated under reduced pressure to give 2.4 g of the desired product containing potassium chloride as a white solid.

m. p.; 257-263° C.

### Step 4

### Preparation of tert-butyl 3-chloro-1-(pyridin-3-yl)-1H-pyrazol-4-ylcarbamate

2.40 g of 3-chloro-1-(pyridin-3-yl)-1H-pyrazole-4-carboxylic acid in 20 ml of 2-methyl-2-propanol was mixed with 2.45 g of triethylamine and 2.00 g of diphenylphosphoryl azide at room temperature successively and refluxed with 2-methyl-2-propanol for 4 hours with stirring. After the reaction, the reaction mixture was allowed to cool to room temperature, poured into ice-water and extracted with 50 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (1:1) as the eluent to give 0.86 g of the desired product as a white solid.

m. p.; 125-128° C.

### Synthetic Example 8

### Preparation of 1-{1-(pyridin-3-yl)-1H-pyrazol-4-yl}-5,6-dihydropyridin-2(1H)-one and 1-{1-(pyridin-3-yl)-1H-pyrazol-4-yl}-1,6-dihydropyridin-2(1H)-one (Compound No. 1-047 of the present invention)

0.30 g of 3-bromo-1-{1-(pyridin-3-yl)-1H-pyrazol-4-yl}piperidin-2-one in 4.0 ml of 1,4-dioxane was mixed with 0.19 g of 1,8-diazabicyclo[5.4.0]-7-undecene and stirred at 60° C. for 2 hours. After the reaction, the reaction mixture was mixed with 10 ml of water and extracted with 20 ml of chloroform. The resulting organic layer was dried over anhydrous sodium sulfate, and the solvent was evaporated under reduced pressure. The resulting residue was purified by preparative medium pressure liquid chromatography using n-hexane-ethyl acetate (with a gradient of from 9:1 to 1:3) as the eluent to give 0.14 g of a 5:1 mixture of 1-{1-(pyridin-3-yl)-1H-pyrazol-4-yl}-5,6-dihydropyridin-2(1H)-one and 1-{1-(pyridin-3-yl)-1H-pyrazol-4-yl}-1,6-dihydropyridin-2(1H)-one as the desired product in the form of a white solid.

¹H NMR (CDCl₃, Me₄Si, 300 MHz) of 1-{1-(pyridin-3-yl)-1H-pyrazol-4-yl}-5,6-dihydropyridin-2(1H)-one

δ9.02 (d, J=2.4 Hz, 1H), 8.61 (s, 1H), 8.55 (dd, J=4.8, 1.2 Hz, 1H), 8.05 (ddd, J=8.1, 2.4, 1.2 Hz, 1H), 7.81 (s, 1H), 7.41 (dd, J=8.1, 4.8 Hz, 1H), 6.69-6.74 (m, 1H), 6.10 (d, J=9.9 Hz, 1H), 3.92 (t, J=6.9 Hz, 2H), 2.57-2.63 (m, 2H)

¹H NMR (CDCl₃, Me₄Si, 300 MHz) of 1-{1-(pyridin-3-yl)-1H-pyrazol-4-yl}-1,6-dihydropyridin-2(1H)-one

δ9.02 (d, J=2.4 Hz, 1H), 8.72 (s, 1H), 8.55 (dd, J=4.8, 1.2 Hz, 1H), 8.05 (ddd, J=8.1, 2.4, 1.2 Hz, 1H), 7.84 (s, 1H), 7.41 (dd, J=8.1, 4.8 Hz, 1H), 6.69-6.74 (m, 1H), 5.88-5.90 (m, 1H), 4.37-4.40 (m, 1H), 3.17-3.19 (m, 2H)

### Synthetic Example 9

### Preparation of 1-{1-(pyridin-3-yl)-1H-pyrazol-4-yl}piperidin-2-one (Compound No. 1-034 of the present invention)

To 108 mg of a mixture of 1-{1-(pyridin-3-yl)-1H-pyrazol-4-yl}-5,6-dihydropyridin-2(1H)-one and 1-{1-(pyridin-3-yl)-1H-pyrazol-4-yl}-1,6-dihydropyridin-2(1H)-one in 2.5 ml of tetrahydrofuran, 12.3 mg of 5 wt % palladium-activated carbon was added. After the addition, the atmosphere in the reaction vessel was replaced by hydrogen gas, and the reaction mixture was stirred at room temperature for 2 days. After the reaction, the palladium-activated carbon in the reaction mixture was filtered off through Celite, and the filtrate was evaporated under reduced pressure to remove the solvent to give 92.5 mg of the desired product as a white solid.

¹H NMR (CDCl₃, Me₄Si, 300 MHz) δ9.02 (d, J=2.4 Hz, 1H), 8.65 (s, 1H), 8.54 (dd, J=4.8, 1.5 Hz, 1H), 8.05 (ddd, J=8.4, 2.4, 1.5 Hz, 1H), 7.82 (s, 1H), 7.41 (dd, J=8.4, 4.8 Hz, 1H), 3.76 (t, J=6.3 Hz, 2H), 2.61 (t, J=6.3 Hz, 2H), 2.06-1.98 (m, 2H), 1.95-1.87 (m, 2H)

### Synthetic Example 10

### Preparation of N,2-dimethyl-3-(methylthio)-N-{1-(pyridin-3-yl)-1H-pyrazol-4-yl}propanamide (Compound No. 1-043 of the present invention)

170 mg of N-methyl-1-(pyridin-3-yl)-1H-pyrazole-4-amine dihydrochloride in 5 ml of dichloromethane was mixed with 348 mg of triethylamine, 206 mg of 2-methyl-3-(methylthio)propanoic acid, 265 mg of 1-ethyl-3-(3-dimethylaminopropyl)carbodiimide hydrochloride and 10 mg of 4-dimethylaminopyridine successively and stirred at room temperature for 12 hours. After the reaction, the reaction mixture was mixed with 5 ml of water and extracted with 5 ml of dichloromethane. The resulting organic layer was washed with saturated aqueous sodium chloride and dried over anhydrous sodium sulfate, and the solvent was evaporated under reduced pressure. The resulting residue was purified by preparative medium pressure liquid chromatography using n-hexane-ethyl acetate (1:1) as the eluent to give 173 mg of the desired product (a 3:1 mixture of two isomers having the same structure) as a yellow oil.

(The compound contained in a ratio of 3) ¹H NMR (CDCl₃, Me₄Si, 300 MHz) δ8.97 (d, J=2.4 Hz, 1H), 8.59 (d, J=4.8 Hz, 1H), 8.08-7.99 (m, 1H), 8.01 (s, 1H), 7.72 (s, 1H), 7.44 (dd, J=4.8, 8.1 Hz, 1H), 3.29 (s, 3H), 3.06-2.82 (m, 2H), 2.48-2.40 (m, 1H), 2.01 (s, 3H), 1.12 (d, J=6.3 Hz, 3H).
(The compound contained in a ratio of 1) ¹H NMR (CDCl₃, Me₄Si, 300 MHz) δ9.02-8.96 (m, 1H), 8.66 (s, 1H), 8.57-8.51 (m, 1H), 8.08-7.99 (m, 1H), 7.78 (s, 1H), 7.42-7.35 (m, 1H), 3.51 (s, 3H), 3.19-2.82 (m, 2H), 2.60 (dd, J=6.0, 13.2 Hz, 1H), 2.14 (s, 3H), 1.30 (d, J=6.9 Hz, 3H).

### Synthetic Example 11

### Preparation of N-methyl-N-{3-chloro-1-(pyridin-3-yl)-1H-pyrazol-4-yl}ethenesulfonamide (Compound No. 2-003 of the present invention)

To 200 mg of 3-chloro-N-methyl-1-(pyridin-3-yl)-1H-pyrazole-4-amine dihydrochloride in 3 ml of ethylene dichloride, 281 mg of pyridine and 139 mg of 2-chloroethanesulfonyl chloride in 1 ml of ethylene dichloride were added dropwise at 0° C., and the reaction mixture was stirred at room temperature for 16 hours. After the reaction, the reaction mixture was mixed with 5 ml of water and extracted with 10 ml of ethyl acetate. The resulting organic layer was washed with saturated aqueous sodium chloride and dried over anhydrous sodium sulfate, and the solvent was evaporated under reduced pressure to give 150 mg of the desired product as a yellow solid.

¹H NMR (CDCl₃, Me₄Si, 300 MHz) δ8.94 (d, J=2.7 Hz, 1H), 8.58 (dd, J=4.5 Hz, 1.5 Hz, 1H), 8.15 (s, 1H), 7.99 (ddd, J=8.4 Hz, 4.5 Hz, 1.5 Hz, 1H), 7.42 (dd, J=8.4 Hz, 4.5 Hz, 1H), 6.63 (dd, J=16.5 Hz, 9.9 Hz, 1H), 6.28 (d, J=16.5 Hz, 1H), 6.09 (d, J=9.9 Hz, 1H), 3.28 (s, 3H)

### Synthetic Example 12

### Preparation of N-methyl-N-{3-chloro-1-(pyridin-3-yl)-1H-pyrazol-4-yl}-2-methylthioethanesulfonamide (Compound No. 2-002 of the present invention)

150 mg of N-methyl-N-{3-chloro-1-(pyridin-3-yl)-1H-pyrazol-4-yl}ethenesulfonamide was dissolved in 2 ml of 4 M aqueous potassium hydroxide and 1.5 ml of tetrahydrofuran, mixed with 220 mg of S,S′-dimethyl dithiocarbonate and stirred at room temperature for 8 hours. After the reaction, the reaction mixture was mixed with 5 ml of water and extracted with 10 ml of ethyl acetate. The resulting organic layer was washed with saturated aqueous sodium chloride and dried over anhydrous sodium sulfate, and the solvent was evaporated under reduced pressure. The resulting residue was purified by preparative medium pressure liquid chromatography using n-hexane-ethyl acetate (with a gradient of from 1:1 to 0:1) as the eluent to give 120 mg of the desired product as a white solid.

¹H NMR (CDCl₃, Me₄Si, 300 MHz) δ8.94 (d, J=2.7 Hz, 1H), 8.60-8.55 (m, 1H), 8.15 (s, 1H), 8.05-7.95 (m, 1H), 7.50-7.35 (m, 1H), 3.36 (s, 3H), 3.40-3.30 (m, 2H), 3.00-2.85 (m, 2H), 2.16 (s, 3H)

m. p.; 101-103° C.

### Synthetic Example 13

### Preparation of N-{3-chloro-1-(pyridin-3-yl)-1H-pyrazol-4-yl}-N-methyl-4,5-dihydrothiazole-2-amine (Compound No. 3-001 of the present invention)

160 mg of 3-chloro-N-methyl-1-(pyridin-3-yl)-1H-pyrazole-4-amine was dissolved in 5 ml of acetonitrile, mixed with 159 mg of potassium carbonate and 140 mg of 2-chloroethyl isothiocyanate successively and refluxed with acetonitrile for 2 hours with stirring. After the reaction, the reaction mixture was mixed with 5 ml of water and extracted with 10 ml of ethyl acetate. The resulting organic layer was washed with saturated aqueous sodium chloride and dried over anhydrous sodium sulfate. The resulting residue was purified by preparative medium pressure liquid chromatography using ethyl acetate-methanol (with a gradient of from 1:0 to 4:1) to give 140 mg of the desired product as a white solid.

¹H NMR (CDCl₃, Me₄Si, 300 MHz) δ8.92 (d, J=2.4 Hz, 1H), 8.57 (d, J=2.4 Hz, 1H), 8.05 (s, 1H), 8.05-7.95 (m, 1H), 7.45-7.35 (m, 1H), 4.13 (t, J=7.2 Hz, 2H), 3.36 (s, 3H), 3.35 (t, J=7.2 Hz, 2H)

### Synthetic Example 14

### Preparation of methyl N-3-methyl-1-(pyridin-3-yl)-1H-pyrazol-4-yl acetimidate (Compound No. 3-002 of the present invention)

1.5 g of 1-(pyridin-3-yl)-1H-pyrazole-4-amine dihydrochloride in 10 ml of trimethyl orthoformate was mixed with 0.01 g of p-toluenesulfonic acid and stirred at 120° C. for 2 hours. After the reaction, the solvent was evaporated under reduced pressure, and the resulting residue was purified by preparative medium pressure liquid chromatography using ethyl acetate-methanol (with a gradient of from 1:0 to 4:1) as the eluent to obtain 180 mg of the desired product as a white solid.

m.p.; 90-92° C.

### Synthetic Example 15

### Preparation of N-methyl-N-[1-{3-methyl-1-(pyridin-3-yl)-1H-pyrazol-4-ylimino}ethyl]-2-(methylthio)acetamide (Compound No. 3-004 of the present invention)

### Step 1

### Preparation of N-methyl-N′-{3-methyl-1-(pyridin-3-yl)-1H-pyrazol-4-yl)acetimidate

160 mg of methyl N-3-methyl-1-(pyridin-3-yl)-1H-pyrazol-4-yl acetimidate was mixed with 4 ml of 9.8 mol/L methylamine in methanol, and the mixture was stirred in a sealed tube at 100° C. for 2 hours. After the reaction, the solvent was evaporated under reduced pressure to obtain 160 mg of the desired product as a brown oil. The oil was used in the next step without purification.

### Step 2

### Preparation of N-methyl-N-[1-{3-methyl-1-(pyridin-3-yl)-1H-pyrazol-4-ylimino}ethyl]-2-(methylthio)acetamide

160 mg of N-methyl-N′-{3-methyl-1-(pyridin-3-yl)-1H-pyrazol-4-yl)acetimidate in 6 ml of dichloromethane 6 ml was mixed with 104 mg of triethylamine, 148 mg of 3-(methylthio)acetic acid, 200 mg of 1-ethyl-3-(3-dimethylaminopropyl)carbodiimide hydrochloride and 10 mg of 4-dimethylaminopyridine successively, and the reaction mixture was stirred at room temperature for 20 hours. After the reaction, the reaction mixture was mixed with 10 ml of water and extracted with 10 ml of ethyl acetate. The resulting organic layer was dried over anhydrous sodium sulfate, and the solvent was evaporated under reduced pressure. The resulting residue was purified by preparative medium pressure liquid chromatography using ethyl acetate-methanol (with a gradient of from 1:0 to 4:1) as the eluent to obtain 98 mg of the desired product as light brown crystals.

m. p.; 54-57° C.

### Synthetic Example 16

### Preparation of 4-chloro-N-{3-chloro-1-(pyridin-3-yl)-1H-pyrazol-4-yl}butanamide (Compound No. 1-131 of the present invention)

To 1.5 g of 3-chloro-1-(pyridin-3-yl)-1H-pyrazole-4-amine dihydrochloride in 25 ml of dichloromethane, 2.0 g of triethylamine and 0.95 g of 4-chlorobutanoyl chloride were added at 0° C. dropwise successively, and the resulting reaction mixture was stirred at room temperature for 1 hour. After the reaction, the reaction mixture was mixed with 25 ml of water and extracted with 50 ml of chloroform. The resulting organic layer was washed with saturated aqueous sodium chloride and then dried over anhydrous sodium sulfate. The solvent was evaporated under reduced pressure, and the resulting residue was purified by preparative medium pressure liquid chromatography using n-hexane-ethyl acetate (with a gradient of from 4:1 to 1:3) as the eluent to obtain 1.47 g of the desired product as a white solid.

m. p.; 97-98° C.

### Synthetic Example 17

### Preparation of 1-{3-chloro-1-(pyridin-3-yl)-1H-pyrazol-4-yl}pyrrolidin-2-one (Compound No. 1-132 of the present invention)

To 1.45 g of 4-chloro-N-{3-chloro-1-(pyridin-3-yl)-1H-pyrazol-4-yl}butanamide in 20 ml of tetrahydrofuran, 0.22 g of 60 wt % sodium hydride (dispersed in mineral oil) was added at 0° C. After the addition, the reaction mixture was stirred at room temperature for 2 hours. After the reaction, the reaction mixture was mixed with 20 ml of water and extracted with 20 ml of ethyl acetate. The resulting organic layer was washed with saturated aqueous sodium chloride and then dried over anhydrous sodium sulfate, and the solvent was evaporated under reduced pressure. The resulting solid was washed with diisopropyl ether to obtain 1.14 g of the desired product as a yellow solid.

m. p.; 109-110° C.

### Synthetic Example 18

### Preparation of 1-{3-chloro-1-(pyridin-3-yl)-1H-pyrazol-4-yl}-3-methylenepyrrolidin-2-one (Compound No. 1-133 of the present invention)

500 mg of 1-{3-chloro-1-(pyridin-3-yl)-1H-pyrazol-4-yl}pyrrolidin-2-one in 5 ml of dichloromethane was mixed with 290 mg of triethylamine and 550 mg of trimethylsilyl trifluoromethansulfonate at 0° C., and the reaction mixture was stirred 45 minutes. After the stirring, the reaction mixture was mixed with 460 mg of dimethylmethyleneammonium iodide and stirred at room temperature overnight. After the stirring, the reaction mixture was stirred with 10 ml of dichloromethane and 11 ml of 1 N aqueous hydrochloric acid for 10 minutes, then adjusted to pH 14 with 2 N aqueous sodium hydroxide and extracted with 15 ml of dichloromethane. The resulting organic layer was washed with saturated aqueous sodium chloride and then dried over anhydrous sodium sulfate, and the solvent was evaporated under reduced pressure. The resulting residue was purified by preparative medium pressure liquid chromatography using n-hexane-ethyl acetate (with a gradient of from 1:1 to 0:1) as the eluent to obtain 230 mg of the desired product as a white solid.

m. p.; 117-118° C.

### Synthetic Example 19

### Preparation of 1-{3-chloro-1-(pyridin-3-yl)-1H-pyrazol-4-yl}-3-(methylthiomethyl)pyrrolidin-2-one (Compound No. 1-141 of the present invention)

187 mg of 1-{3-chloro-1-(pyridin-3-yl)-1H-pyrazol-4-yl}-3-methylenepyrrolidin-2-one in 3 ml of tetrahydrofuran was mixed with 1 ml of 30% aqueous potassium hydroxide and then with 58 mg of S,S′-dimethyl dithiocarbonate and refluxed with tetrahydrofuran for 3 hours with stirring. After the reaction, the reaction mixture was mixed with 10 ml of water and extracted with 20 ml of chloroform. The resulting organic layer was washed with saturated aqueous sodium chloride and then dried over anhydrous sodium sulfate, and the solvent was evaporated under reduced pressure. The resulting residue was purified by preparative medium pressure liquid chromatography using n-hexane-ethyl acetate (with a gradient of from 3:1 to 0:1) to obtain 214 mg of the desired product as a white solid.

m. p.; 83-84° C.

### Synthetic Example 20

### Preparation of N-{3-chloro-1-(pyridin-3-yl)-1H-pyrazol-4-yl}-N-ethyl-2-(methoxyimino)-3-(methylthio)propanamide (Compound No. 1-143 of the present invention)

### Step 1

### Preparation of ethyl 2-(methoxyimino)-3-(methylthio)propanoate (Compound No. D-001)

2.0 g of ethyl 3-bromo-2-(methoxyimino)propanoate in 30 ml of N,N-dimethylformamide was mixed with 750 mg of sodium methylmercaptan and stirred at room temperature for 2 hours. After the stirring, the reaction mixture was heated to 80° C. and stirred at the same temperature for another one hour. After the reaction, the reaction mixture was mixed with 30 ml of water and extracted with 50 ml of ethyl acetate. The resulting organic layer was washed with saturated aqueous sodium hydrogen carbonate and then with saturated aqueous sodium chloride and dried over anhydrous sodium sulfate. The solvent was evaporated under reduced pressure to obtain 2 g of the desired product as a pale yellow oil.

¹H NMR (CDCl₃, Me₄Si, 300 MHz) δ4.34 (q, J=7.2 Hz, 2H), 4.04 (s, 3H), 3.55 (s, 2H), 2.10 (s, 3H), 1.35 (t, J=7.2 Hz, 3H)

### Step 2

### Preparation of 2-(methoxyimino)-3-(methylthio)propanoic acid (Compound No. E-001)

1.84 g of ethyl 2-(methoxyimino)-3-(methylthio)propanoate in a mixture of 10 ml of water and 10 ml of ethanol was mixed with 430 mg of sodium hydroxide and stirred at room temperature for 2 days. After the reaction, the reaction solution was adjusted to pH 2 with 1 N aqueous hydrochloric acid and extracted with 20 ml of ethyl acetate twice. The resulting organic layer was washed with saturated aqueous sodium chloride and dried over anhydrous sodium sulfate, and the solvent was evaporated under reduced pressure. The resulting solid was washed with 6 ml of a mixture of hexane:isopropyl ether (5:1) to obtain 0.4 g of the desired product as a pale yellow solid.

¹H NMR (CDCl₃, Me₄Si, 300 MHz) δ4.08 (s, 3H), 3.53 (s, 2H), 2.14 (s, 3H)

### Step 3

### Preparation of N-{3-chloro-1-(pyridin-3-yl)-1H-pyrazol-4-yl}-N-ethyl-2-(methoxyimino)-3-(methylthio)propanamide

415 mg of 2-(methoxyimino)-3-(methylthio)propanoic acid in 3 ml of dichloromethane was mixed with 429 mg of oxalyl chloride and 10 mg of N,N-dimethylformamide successively and stirred at room temperature for 1 hour. After the stirring, the reaction solution was added dropwise to a separately prepared solution of 500 mg of 3-chloro-N-ethyl-1-(pyridin-3-yl)-1H-pyrazole-4-amine dihydrochloride and 668 mg of pyridine in 3 ml of ethylene dichloride, and the resulting reaction mixture was stirred at room temperature for 30 minutes. After the reaction, the reaction mixture was washed with 10 ml of water and 10 ml of saturated aqueous sodium hydrogen carbonate and dried over anhydrous sodium sulfate, and the solvent was evaporated under reduced pressure. The resulting residue was purified by preparative medium pressure liquid chromatography using n-hexane-ethyl acetate (1:4) as the eluent to obtain 534 mg of the desired product as a pale yellow oil.

¹H NMR (CDCl₃, Me₄Si, 300 MHz) δ8.90 (d, J=2.4 Hz, 1H), 8.58 (dd, J=4.8, 1.2 Hz, 1H), 8.04-7.95 (m, 1H), 7.98 (s, 1H), 7.43 (dd, J=8.1, 4.8 Hz, 1H), 3.78 (q, J=7.2 Hz, 2H), 3.68 (s, 3H), 3.52 (s, 2H), 1.90 (s, 3H), 1.22 (t, J=7.2 Hz, 3H)

### Synthetic Example 21

### Preparation of tert-butyl 3-chloro-1-(pyridin-3-yl)-1H-pyrazol-4-yl(methyl)carbamate (Compound No. 1-037 of the present invention)

To 0.34 g of 60 wt % sodium hydride (dispersed in mineral oil) in 25 ml of N,N-dimethylformamide, 2.2 g of tert-butyl3-chloro-1-(pyridin-3-yl)-1H-pyrazol-4-ylcarbamate was added under cooling with ice. After the addition, the reaction mixture was warmed to room temperature and stirred at the same temperature for 30 minutes. After the stirring, the reaction mixture was mixed with 1.17 g of methyl iodide and stirred at room temperature for another 30 minutes. After the reaction, the reaction mixture was poured into ice-water and extracted with 100 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (with a gradient of from 9:1 to 1:1) as the eluent to obtain 2.01 g of the desired product as a yellow oil.

¹H NMR (CDCl₃, Me₄Si, 300 MHz) δ8.90 (d, J=2.4 Hz, 1H), 8.56 (dd, J=4.8, 1.5 Hz, 1H), 8.00 (ddd, J=8.1, 2.4, 1.5 Hz, 1H), 7.90 (s, 1H), 7.40 (dd, J=8.1, 4.8 Hz, 1H), 3.22 (s, 3H), 1.46 (s, 9H)

### Synthetic Example 22

### Preparation of N-{3-chloro-1-(pyridin-3-yl)-1H-pyrazol-4-yl}-N-methyl-3-(methylthio)propanamide (Compound No. 1-038 of the present invention)

### Step 1

### Preparation of 3-chloro-N-methyl-1-(pyridin-3-yl)-1H-pyrazole-4-amine dihydrochloride (Compound No. A-003)

1.93 g of tert-butyl 3-chloro-1-(pyridin-3-yl)-1H-pyrazol-4-yl(methyl)carbamate in 25 ml of 1,4-dioxane was mixed with 10 ml of about 4 M hydrogen chloride in 1,4-dioxane and stirred at room temperature for 2 days. After the reaction, the solid precipitated in the reaction solution was collected by filtration under reduced pressure and dried under reduced pressure to obtain 1.63 g of the desired product as a white solid.

m. p.; 159-160° C.

### Step 2

### Preparation of N-{3-chloro-1-(pyridin-3-yl)-1H-pyrazol-4-yl}-N-methyl-3-(methylthio)propanamide

800 mg of 3-chloro-N-methyl-1-(pyridin-3-yl)-1H-pyrazole-4-amine dihydrochloride in 10 ml of dichloromethane was mixed with 720 mg of triethylamine, 685 mg of 3-(methylthio)propanoic acid, 1.63 of g 1-ethyl-3-(3-dimethylaminopropyl)carbodiimide hydrochloride and 695 mg of 4-dimethylaminopyridine successively and stirred at room temperature for 12 hours. After the reaction, the reaction mixture was mixed with 20 ml of water and extracted with 30 ml of chloroform. The resulting organic layer was dried over anhydrous sodium sulfate, and the solvent was evaporated under reduced pressure. The resulting residue was purified by preparative medium pressure liquid chromatography using n-hexane-ethyl acetate (with a gradient of from 3:1 to 0:1) as the eluent to obtain 463 mg of the desired product as a colorless oil.

¹H NMR (CDCl₃, Me₄Si, 300 MHz) δ8.94 (d, J=2.7 Hz, 1H), 8.63 (dd, J=4.5, 1.2 Hz, 1H), 8.04 (ddd, J=8.1, 2.7, 1.2 Hz, 1H), 8.00 (s, 1H), 7.46 (dd, J=8.1, 4.5 Hz, 1H), 3.25 (s, 3H), 2.80 (t, J=7.2 Hz, 2H), 2.48 (t, J=7.2 Hz, 2H), 2.07 (s, 3H)

### Synthetic Example 23

### Preparation of N-{3-chloro-1-(pyridin-3-yl)-1H-pyrazol-4-yl}-N-methyl-3-(methylsulfinyl)propanamide (Compound No. 1-051 of the present invention)

153 mg of N-{3-chloro-1-(pyridin-3-yl)-1H-pyrazol-4-yl}-N-methyl-3-(methylthio)propanamide in 3 ml of acetic acid wax mixed with 56 mg of 30 wt % aqueous hydrogen peroxide and stirred at room temperature for 12 hours. After the reaction, the reaction mixture was mixed with 1 ml of saturated aqueous sodium hydrogensulfite, and the solvent was evaporated under reduced pressure. The resulting residue was purified by preparative medium pressure liquid chromatography using ethyl acetate-methanol (5:1) as the eluent to obtain 145 mg of the desired product as a colorless oil.

¹H NMR (CDCl₃, Me₄Si, 300 MHz) δ8.96 (d, J=2.4 Hz, 1H), 8.63 (dd, J=4.8, 1.2 Hz, 1H), 8.07 (s, 1H), 8.03 (ddd, J=8.4, 2.4, 1.2 Hz, 1H), 7.46 (dd, J=8.4, 4.8 Hz, 1H), 3.27 (s, 3H), 3.11-3.21 (m, 1H), 2.84-2.93 (m, 1H), 2.66-2.76 (m, 2H), 2.60 (s, 3H)

### Synthetic Example 24

### Preparation of N-{3-chloro-1-(pyridin-3-yl)-1H-pyrazol-4-yl}-N-methyl-3-(methylsulfonyl)propanamide (Compound No. 1-052 of the present invention)

158 mg of N-{3-chloro-1-(pyridin-3-yl)-1H-pyrazol-4-yl}-N-methyl-3-(methylthio)propanamide in 3 ml of acetic acid was mixed with 56 mg of 30 wt % aqueous hydrogen peroxide and 8 mg of sodium tungstate dihydride and stirred at room temperature for 12 hours. After the reaction, the reaction mixture was mixed with 1 ml of aqueous saturated sodium hydrogensulfite, and the solvent was evaporated under reduced pressure. The resulting residue was purified by preparative medium pressure liquid chromatography using n-hexane-ethyl acetate (with a gradient of from 1:1 to 0:1) as the eluent to obtain 160 mg of the desired product as a white solid.

m. p.; 90-91° C.

### Synthetic Example 25

### Preparation of N-{3-chloro-1-(pyridin-3-yl)-1H-pyrazol-4-yl}-N,2,2-trimethyl-3-(methylthio)propanamide (Compound No. 1-134 of the present invention)

### Step 1

### Preparation of 3-chloro-N-methyl-1-(pyridin-3-yl)-1H-pyrazole-4-amine (Compound No. B-003)

17.3 g of 3-chloro-N-methyl-1-(pyridin-3-yl)-1H-pyrazole-4-amine dihydrochloride in 200 ml of chloroform and 100 ml of water was mixed with 25.3 g of potassium carbonate and stirred at room temperature for 15 minutes. After the reaction, the reaction solution was separated, and the resulting organic layer was washed with saturated aqueous sodium chloride and then dried over anhydrous sodium sulfate. The solvent was evaporated under reduced pressure, and the resulting solid was washed with diisopropyl ether to obtain 11.1 g of the desired product as a pale yellow solid.

¹H NMR (CDCl₃, Me₄Si, 300 MHz) δ8.87 (d, J=2.7 Hz, 1H), 8.46 (dd, J=4.5 Hz, 1.8 Hz, 1H), 7.94 (ddd, J=8.1 Hz, 2.7 Hz, 1.8 Hz, 1H), 7.34 (dd, J=8.1 Hz, 4.5 Hz, 1H), 7.32 (s, 1H), 3.11 (brs, 1H), 2.85 (s, 3H)

m. p. 105-106° C.

### Step 2

### Preparation of N-{3-chloro-1-(pyridin-3-yl)-1H-pyrazol-4-yl}-N,2,2-trimethyl-3-(methylthio)propanamide

500 mg of 3-chloro-N-methyl-1-(pyridin-3-yl)-1H-pyrazole-4-amine in 8 ml of dichloromethane was mixed with 484 mg of triethylamine and cooled to 0° C. After the cooling, 622 mg of 2,2-dimethyl-3-(methylthio)propanoyl chloride was added dropwise to the reaction solution, and the reaction solution was stirred at room temperature for 12 hours. After the reaction, the reaction mixture was mixed with 5 ml of water and extracted with 10 ml of chloroform. The resulting organic layer was dried over anhydrous sodium sulfate, and the solvent was evaporated under reduced pressure. The resulting residue was purified by preparative medium pressure liquid chromatography using n-hexane-ethyl acetate (with a gradient of from 1:1 to 1:4) as the eluent to obtain 180 mg of the desired product as a colorless oil.

¹H NMR (CDCl₃, Me₄Si, 300 MHz) δ8.95 (d, J=1.5 Hz, 1H), 8.60 (d, J=4.8 Hz, 1H), 8.14 (s, 1H), 8.04 (d, J=8.4 Hz, 1H), 7.45 (dd, J=8.4 Hz, 4.8 Hz, 1H), 3.21 (s, 3H), 2.72 (s, 2H), 2.14 (s, 3H), 1.21 (s, 6H)

### Synthetic Example 26

### Preparation of tert-butyl 3-chloro-1-(pyridin-3-yl)-1H-pyrazol-4-yl(ethyl)carbamate (Compound No. 1-056 of the present invention)

To 0.15 g of 60 wt % sodium hydride (dispersed in mineral oil) in 10 ml of N,N-dimethylformamide, 0.95 g of tert-butyl 3-chloro-1-(pyridin-3-yl)-1H-pyrazol-4-ylcarbamate was added under cooling with ice. After the addition, the reaction mixture was warmed to room temperature and stirred at the same temperature for 30 minutes. After the stirring, the reaction mixture was mixed with 0.55 g of ethyl iodide and stirred at room temperature for another 1 hour. After the reaction, the reaction mixture was poured into ice-water and extracted with 100 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (2:1) as the eluent to obtain 1.06 g of the desired product as a yellow solid.

m. p.; 55-58° C.

### Synthetic Example 27

### Preparation of N-{3-chloro-1-(pyridin-3-yl)-1H-pyrazol-4-yl}-N-ethylacrylamide (Compound No. 1-152 of the present invention)

### Step 1

### Preparation of 3-chloro-N-ethyl-1-(pyridin-3-yl)-1H-pyrazole-4-amine dihydrochloride (Compound No. A-004)

1.0 g of tert-butyl 3-chloro-1-(pyridin-3-yl)-1H-pyrazol-4-yl(ethyl)carbamate in 20 ml of 1,4-dioxane was mixed with 8.1 ml of about 4 M hydrogen chloride in 1,4-dioxane and stirred at room temperature for 12 hours. After the reaction, the solid precipitated in the reaction solution was collected by filtration under reduced pressure and dried under reduced pressure to obtain 0.85 g of the desired product as a white solid.

m. p.; 132-133° C.

### Step 2

### Preparation of 3-chloro-N-ethyl-1-(pyridin-3-yl)-1H-pyrazole-4-amine (Compound No. B-004)

7.4 ml of 1 N aqueous sodium hydroxide was mixed with 1 g of 3-chloro-N-ethyl-1-(pyridin-3-yl)-1H-pyrazole-4-amine dihydrochloride and stirred at room temperature for 3 hours. After the reaction, the reaction mixture was extracted with dichloromethane (20 ml×3). The resulting organic layer was dried over anhydrous sodium sulfate, and the solvent was evaporated under reduced pressure to obtain 660 mg of the desired product as a white solid.

¹H NMR (CDCl₃, Me₄Si, 300 MHz) δ8.86 (d, J=2.7 Hz, 1H), 8.46 (d, J=4.8, 1.2 Hz, 1H), 8.00-7.90 (m, 1H), 7.45 (ddd, J=8.1, 4.8, 1.2 Hz, 1H), 7.33 (s, 1H), 3.01 (q, J=7.2 Hz, 2H), 3.00 (brs, 1H), 1.31 (t, J=7.2 Hz, 3H)

m. p.; 89-90° C.

### Step 3

### Preparation of N-{3-chloro-1-(pyridin-3-yl)-1H-pyrazol-4-yl}-N-ethylacrylamide

380 mg of 3-chloro-N-ethyl-1-(pyridin-3-yl)-1H-pyrazole-4-amine in 8.5 ml of dichloromethane was mixed with 8.5 ml of saturated aqueous sodium hydrogencarbonate and then with 230 mg of acryloyl chloride and stirred at room temperature for 20 minutes. After the reaction, the reaction mixture was extracted with dichloromethane (20 ml×2). The resulting organic layer was dried over anhydrous sodium sulfate, and the solvent was evaporated under reduced pressure. The resulting residue was purified by preparative medium pressure liquid chromatography using n-hexane-ethyl acetate (with a gradient of from 9:1 to 1:1) to obtain 320 mg of the desired product as a white solid.

¹H NMR (CDCl₃, Me₄Si, 300 MHz) δ8.94 (d, J=2.7 Hz, 1H), 8.61 (dd, J=4.8, 1.5 Hz, 1H), 8.00-8.10 (m, 1H), 7.95 (s, 1H), 7.45 (dd, J=8.6, 4.8 Hz, 1H), 6.43 (dd, J=16.8, 1.8 Hz, 1H), 6.17 (dd, 16.8, 10.4 Hz, 1H), 5.63 (dd, 10.4, 1.8 Hz, 1H), 3.77 (q, 7.1 Hz, 2H). 1.20 (t, 7.1 Hz, 3H)

m. p.; 67-68° C.

### Synthetic Example 28

### Preparation of N-{3-chloro-1-(pyridin-3-yl)-1H-pyrazol-4-yl}-N-{3-(methylthio)propyl}acetamide (Compound No. 1-153 of the present invention)

### Step 1

### Preparation of 3-chloro-N-{3-(methylthio)propyl}-1-(pyridin-3-yl)-1H-pyrazole-4-amine (Compound No. C-001)

500 mg of N-{3-chloro-1-(pyridin-3-yl)-1H-pyrazol-4-yl}-3-(methylthio)propanamide in 15 ml of tetrahydrofuran was mixed with 3.3 ml of 0.98M borane-tetrahydrofuran complex in tetrahydrofuran at room temperature and refluxed with tetrahydrofuran for 2 hours with stirring. After the reaction, the reaction solution was cooled to 0° C., mixed with 15 ml of water and then with 10 ml of saturated aqueous sodium hydrogencarbonate and extracted with 150 ml of ethyl acetate. The resulting organic layer was dried over anhydrous sodium sulfate. The resulting residue was purified by silica gel column chromatography using n-hexane-ethyl acetate (1:1) as the eluent to obtain 256 mg of the desired product as a yellow solid.

¹H NMR (CDCl₃, Me₄Si, 300 MHz) δ8.82 (d, J=1.8 Hz, 1H), 8.42 (d, J=5.4 Hz, 1H), 8.21 (dd, J=8.4, 1.8 Hz, 1H), 7.54 (dd, J=8.4, 5.4 Hz, 1H), 7.34 (s, 1H), 3.23 (brs, 1H), 3.21 (t, J=6.9 Hz, 2H), 2.65 (t, J=6.9 Hz, 2H), 2.15 (s, 3H), 1.96 (tt, J=6.9, 6.9 Hz, 2H)

m. p.; 80-81° C.

### Step 2

### Preparation of N-{3-chloro-1-(pyridin-3-yl)-1H-pyrazol-4-yl}-N-{3-(methylthio)propyl}acetamide

150 mg of 3-chloro-N-{3-(methylthio)propyl}-1-(pyridin-3-yl)-1H-pyrazole-4-amine in 2 ml of dichloromethane was mixed with 1 ml of pyridine and cooled to 0° C. Then, to the reaction solution, 163 mg of acetic anhydride was added dropwise, and the reaction solution was stirred at room temperature for 12 hours. After the reaction, the reaction mixture was mixed with 30 ml of water and extracted with 150 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (1:4) as the eluent to obtain 145 mg of the desired product as a colorless oil.

¹H NMR (CDCl₃, Me₄Si, 300 MHz) δ8.95 (d, J=2.7 Hz, 1H), 8.63 (dd, J=4.8, 1.5 Hz, 1H), 8.05 (ddd, J=8.4, 2.7, 1.5 Hz, 1H), 7.97 (s, 1H), 7.46 (dd, J=8.4, 4.8 Hz, 1H), 3.73 (t, J=7.5 Hz, 2H), 2.54 (t, J=7.2 Hz, 2H), 2.10 (s, 3H), 1.97 (s, 3H), 1.95-1.75 (m, 2H)

### Synthetic Example 29

### Preparation of N-{3-chloro-1-(pyridin-3-yl)-1H-pyrazol-4-yl}-N-ethyl-2-methoxy-3-(methylthio)propanamide (Compound No. 1-160 of the present invention)

### Step 1

### Preparation of methyl 2-methoxy-3-(methylthio)propanoate

2.3 g of methyl 2-methoxyacrylate in 10 ml of acetonitrile was mixed with 1.39 g of sodium methylmercaptan and stirred at room temperature for 1 hour. After the reaction, the reaction mixture was mixed with 10 ml of water and extracted with 10 ml of ethyl acetate. The resulting organic layer was dried over anhydrous sodium sulfate, and the solvent was evaporated under reduced pressure to obtain 1.92 g of the desired product as a pale yellow oil.

¹H NMR (CDCl₃, Me₄Si, 300 MHz) δ63.98 (dd, J=6.6, 5.4 Hz, 1H), 3.79 (s, 3H), 3.45 (s, 3H), 3.95-3.75 (m, 2H), 2.19 (s, 3H)

### Step 2

### Preparation of 2-methoxy-3-(methylthio)propanoic acid

1.92 g of methyl 2-methoxy-3-(methylthio)propanoate in 5 ml of methanol was mixed with 0.94 g of sodium hydroxide and 5 ml of water and stirred at room temperature for 1 hour. After the reaction, the reaction mixture was mixed with 10 ml of water, and the aqueous layer was washed with 10 ml of ethyl acetate, then adjusted to pH 2 with 1 N aqueous hydrogen chloride and extracted with 10 ml of ethyl acetate twice. The resulting organic layer was washed with saturated aqueous sodium chloride and dried over anhydrous sodium sulfate, and the solvent was evaporated under reduced pressure to obtain 270 mg of the desired product as a pale yellow oil.

¹H NMR (CDCl₃, Me₄Si, 300 MHz) δ3.98 (dd, J=6.6, 4.2 Hz, 1H), 3.52 (s, 3H), 3.00-2.80 (m, 2H), 2.21 (s, 3H)

### Step 3

### Preparation of N-{3-chloro-1-(pyridin-3-yl)-1H-pyrazol-4-yl}-N-ethyl-2-methoxy-3-(methylthio)propanamide

420 mg of 3-chloro-N-methyl-1-(pyridin-3-yl)-1H-pyrazole-4-amine dihydrochloride in 2 ml of dichloromethane was mixed with 725 mg of pyridine, 240 mg of 2-methoxy-3-(methylthio)propanoic acid, 555 mg of 1-ethyl-3-(3-dimethylaminopropyl)carbodiimide hydrochloride and 10 mg of 4-dimethylaminopyridine successively and stirred at room temperature for 1 hour. After the reaction, the reaction mixture was mixed with 10 ml of water and extracted with 10 ml of chloroform. The resulting organic layer was dried over anhydrous sodium sulfate, and the solvent was evaporated under reduced pressure. The resulting residue was purified by preparative medium pressure liquid chromatography using n-hexane-ethyl acetate (with a gradient of from 3:1 to 3:7) as the eluent to obtain 245 mg of the desired product as a colorless oil.

### Synthetic Example 30

### Preparation of tert-butyl 3-bromo-1-(pyridin-3-yl)-1H-pyrazol-4-ylcarbamate (Compound No. 1-188 of the present invention)

### Step 1

### Preparation of ethyl 3-amino-1-(pyridin-3-yl)-1H-pyrazole-4-carboxylate

To 20 g of ethyl 3-amino-1H-pyrazole-4-carboxylate in 200 ml of N,N-dimethylformamide, 29.1 g of 3-iodopyridine, 7.4 g of copper (I) iodide and 72.7 g of cesium carbonate were added successively. After the addition, the atmosphere in the reaction vessel was replaced by nitrogen gas, and the reaction mixture was stirred at 100° C. for 10 hours. After the reaction, the reaction mixture was mixed with 800 ml of water and 150 g of sodium chloride, and the precipitated solid was filtered off. The filtrate was extracted with ethyl acetate (800 ml×3) and then with 500 ml of n-butanol. The resulting organic layer was dried over anhydrous sodium sulfate, and the solvent was evaporated under reduced pressure. 200 ml of ethyl acetate was added to the residue, and the precipitated solid was filtered off. The filtrate was evaporated to remove the solvent. 15 ml of chloroform was added to the residue, and the precipitated solid was collected by filtration to obtain 3.1 g of the desired product as a brown solid. The filtrate was purified by silica gel column chromatography using n-hexane-ethyl acetate (with a gradient of from 2:3 to 1:4) as the eluent to obtain 3.0 g of the desired product as a brown solid.

m. p.; 140-141° C.

### Step 2

### Preparation of ethyl 3-bromo-1-(pyridin-3-yl)-1H-pyrazole-4-carboxylate

6.1 g of ethyl 3-amino1-(pyridin-3-yl)-1H-pyrazole-4-carboxylate in 50 ml of acetonitrile was mixed with 11.8 g of copper (II) bromide and stirred at 60° C. After the stirring, 4.1 g of tert-butyl nitrite was added dropwise to the reaction solution, and the reaction solution was stirred at 60° C. for 3 hours. After the reaction, the reaction solution was cooled to 0° C., adjusted to pH 4 with 1 N aqueous sulfuric acid and stirred at room temperature for 1 hour. The reaction solution was further adjusted to pH 7 with 1 N aqueous sodium hydroxide, mixed with 150 ml of water and extracted with 300 ml of ethyl acetate. The resulting organic layer was dried over anhydrous sodium sulfate, and the solvent was evaporated under reduced pressure. The resulting residue was purified by silica gel column chromatography using n-hexane-ethyl acetate (with a gradient of from 3:1 to 1:4) as the eluent to obtain 4.5 g of the desired product as a pale yellow solid.

m. p.; 94-95° C.

### Step 3

### Preparation of 3-bromo-1-(pyridin-3-yl)-1H-pyrazole-4-carboxylic acid

4.5 g of ethyl 3-bromo-1-(pyridin-3-yl)-1H-pyrazole-4-carboxylate in 75 ml of methanol was mixed with 1.7 g of potassium hydroxide and 10 ml of water and stirred at room temperature for 11 hours. After the reaction, the reaction solution was adjusted to pH 4 with 2 N aqueous hydrogen chloride, and the solvent was evaporated under reduced pressure. The resulting residue was mixed with 150 ml of water and extracted with ethyl acetate (200 ml×3). The resulting organic layer was dried over anhydrous sodium sulfate, and the solvent was evaporated under reduced pressure to obtain 4 g of the desired product as a white solid.

m. p.; 270-278° C.

### Step 4

### Preparation of tert-butyl 3-bromo-1-(pyridin-3-yl)-1H-pyrazol-4-ylcarbamate

To 4.0 g of 3-bromo-1-(pyridin-3-yl)-1H-pyrazole-4-carboxyli acid and 15 ml of 2-methyl-2-propanol in 15 ml of toluene, 3.77 g of triethylamine and 6.16 g of diphenylphosphoryl azide were added dropwise at room temperature successively. After the addition, the reaction mixture was refluxed with toluene for 4 hours with stirring. After the reaction, the reaction mixture was allowed to cool to room temperature, poured into ice-water and extracted with 300 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (1:1) as the eluent to obtain 4.0 g of the desired product as a pale yellow solid.

m. p.; 109-112° C.

### Synthetic Example 31

### Preparation of tert-butyl 5-methyl-1-(pyridin-3-yl)-1H-pyrazol-4-ylcarbamate (Compound No. 5-001 of the present invention)

### Step 1

### Preparation of tert-butyl-1-(pyridin-3-yl)hydrazinecarboxylate

To 25 g of 3-iodopyridine in 200 ml of N,N-dimethylformamide, 219.3 g of tert-butyl carbazate, 64.3 g of cesium carbonate, 4.32 g of 1,10-phenanthroline, 1.16 g of copper (I) iodide were added successively. After the addition, the atmosphere in the reaction vessel was replaced by nitrogen gas, and the reaction mixture was stirred at 100° C. for 4 hours. After the reaction, the reaction mixture was allowed to cool to room temperature, and the insolubles in the reaction mixture were filtered off through celite under reduced pressure. After addition of 100 ml of 1 N aqueous sodium hydroxide, the filtrate was extracted with ethyl acetate (200 ml×1). The resulting organic layer was dried over anhydrous sodium sulfate, and the solvent was evaporated under reduced pressure to obtain 25.5 g of the desired product as a brown oil.

¹H NMR (CDCl₃, Me₄Si, 300 MHz) δ8.81 (d, J=2.5 Hz, 1H), 8.32 (dd, J=4.8, 1.2 Hz, 1H), 7.90-7.80 (m, 1H), 7.23 (dd, J=8.4, 4.8 Hz, 1H), 4.43 (brs, 2H), 1.52 (s, 9H).

### Step 2

### Preparation of 3-hydrazinylpyridine hydrochloride

25.5 g of tert-butyl-1-(pyridin-3-yl)hydrazinecarboxylate in 200 ml of 1,4-dioxane was mixed with 122 ml of about 4 M hydrogen chloride in 1,4-dioxane and stirred at room temperature for 12 hours. After the reaction, the crystals precipitated in the reaction solution were collected by filtration to obtain 26.5 g of the desired product as a white solid.

m. p.; 169-175° C.

### Step 3

### Preparation of ethyl 5-methyl-1-(pyridin-3-yl)-1H-pyrazole-4-carboxylate

1.0 g of 3-hydrazinylpyridine in 15 ml of ethanol was mixed with 1.27 g of ethyl 2-dimethylaminomethylene 3-oxobutanoate and refluxed with ethanol for 30 minutes with stirring. After the reaction, the ethanol was evaporated from the reaction mixture, and the resulting residue was diluted with ethyl acetate and washed with water. The resulting organic layer was dried over anhydrous sodium sulfate, and the solvent was evaporated under reduced pressure. The resulting residue was purified by eluting a silica gel column with ethyl acetate to obtain 0.94 g of the desired product as a brown oil.

¹H NMR (CDCl₃, Me₄Si, 300 MHz) δ8.73 (d, J=2.4 Hz, 1H), 8.68 (dd, J=4.8, 1.2 Hz, 1H), 8.06 (s, 1H), 7.80 (ddd, J=8.1, 2.4, 1.2 Hz, 1H), 7.46 (dd, J=8.1, 4.8 Hz, 1H), 4.33 (q, J=7.2 Hz, 2H), 2.59 (s, 3H), 1.37 (t, J=7.2 Hz, 3H)

### Step 4

### Preparation of 5-methyl-1-(pyridin-3-yl)-1H-pyrazole-4-carboxylic acid

3.88 g of ethyl 5-methyl-1-(pyridin-3-yl)-1H-pyrazole-4-carboxylate was dissolved in 30 ml of ethanol, then mixed with 1.41 g of potassium hydroxide and 30 ml of water successively and stirred overnight at room temperature. After the reaction, the ethanol was evaporated, and the resulting residue was adjusted to pH 4 with concentrated hydrochloric acid. The crystals precipitated in the reaction mixture were collected by filtration to obtain 2.82 g of the desired product as a pale yellow solid.

m. p.; 192-212° C.

### Step 5

### Preparation of tert-butyl 5-methyl-1-(pyridin-3-yl)-1H-pyrazol-4-ylcarbamate

To 2.7 g of 5-methyl-1-(pyridin-3-yl)-1H-pyrazole-4-carboxylic acid and 15 ml of 2-methyl-2-propanol in 45 ml of toluene, 3.4 g of triethylamine and 5.5 g of diphenylphosphoryl azide were added at room temperature successively. After the addition, the reaction mixture was stirred at 100° C. for 2 hours. After the reaction, the reaction mixture was allowed to cool to room temperature, poured into ice-water and extracted with 50 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (1:3) as the eluent to obtain 2.9 g of the desired product as a pale yellow solid.

m. p.; 89-91° C.

### Synthetic Example 32

### Preparation of N-{3-chloro-1-(pyridin-3-yl)-1H-pyrazol-4-yl}-N-ethyl-2-(ethylthio)propanamide (Compound No. 1-118 of the present invention)

### Step 1

### Preparation of 1-tert-butyl-5-chloro-1H-pyrazole-4-carboxylic acid

10 g of ethyl 1-tert-butyl-5-chloro-1H-pyrazole-4-carboxylate was dissolved in 50 ml of ethanol, and 2.6 g of sodium hydroxide and 20 ml of water were added successively. After the addition, the reaction mixture was stirred at room temperature overnight. After the reaction, the solvent was evaporated under reduced pressure, and the resulting residue was adjusted to pH 3 with aqueous hydrochloric acid. The solid precipitated in the reaction solution was collected by filtration, washed with water and dried under reduced pressure to obtain 6.8 g of the desired product as a white solid.

m. p.; 132-134° C.

### Step 2

### Preparation of tert-butyl 1-tert-butyl-5-chloro-1H-pyrazol-4-ylcarbamate (Compound No. F-001)

To 5.0 g of 1-tert-butyl-5-chloro-1H-pyrazole-4-carboxylic acid and 40 ml of 2-methyl-2-propanol in 40 ml of toluene, 3.25 g of triethylamine and 7.47 g of diphenylphosphoryl azide were added at room temperature successively. After the addition, the reaction mixture was heated with stirring for 1.5 hours under reflux. After the reaction, the reaction mixture was allowed to cool to room temperature, poured into ice-water and extracted with 100 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (6:1) as the eluent to obtain 6.25 g of the desired product as a pale yellow oil.

¹H NMR (CDCl₃, Me₄Si, 300 MHz) δ7.74 (s, 1H), 6.00 (brs, 1H), 1.72 (s, 9H), 1.50 (s, 9H)

### Step 3

### Preparation of tert-butyl 1-tert-butyl-5-chloro-1H-pyrazol-4-yl(ethyl)carbamate (Compound No. G-001)

To 136 mg of 60 wt % sodium hydride (dispersed in mineral oil) in 5 ml of tetrahydrofuran, 850 mg of tert-butyl 1-tert-butyl-5-chloro-1H-pyrazol-4-ylcarbamate was added under cooling with ice. After the addition, the reaction mixture was warmed to room temperature and stirred for 30 minutes. After the stirring, the reaction mixture was mixed with 532 mg of ethyl iodide and stirred at room temperature for another 1 hour. Then, the reaction solution was mixed with 10 ml of N,N-dimethylformamide and stirred for another 2 hours. After the reaction, the reaction mixture was poured into ice-water and extracted with 40 ml of ethyl acetate. The resulting organic layer was dried over anhydrous sodium sulfate, and the solvent was evaporated under reduced pressure. The resulting residue was purified by preparative medium pressure liquid chromatography using n-hexane-ethyl acetate (9:1) as the eluent to obtain 680 mg of the desired product as a white solid.

m. p.; 50-52° C.

### Step 4

### Preparation of 1-tert-butyl-5-chloro-N-ethyl-1H-pyrazole-4-amine hydrochloride (Compound No. H-001)

0.3 g of tert-butyl 1-tert-butyl-5-chloro-1H-pyrazol-4-yl(ethyl)carbamate in 2 ml of 1,4-dioxane was mixed with 2 ml of about 4 M hydrogen chloride in 1,4-dioxane and stirred at room temperature overnight. After the reaction, the solvent was evaporated under reduced pressure to obtain 0.27 g of the desired product as a white solid.

¹H NMR (CDCl₃, Me₄Si, 300 MHz) δ7.78 (s, 1H), 3.69 (s, 1H), 3.41 (q, J=7.2 Hz, 2H), 1.69 (s, 9H), 1.42 (t, J=7.2 Hz, 3H)

### Step 5

### Preparation of N-(1-tert-butyl-5-chloro-1H-pyrazol-4-yl)-N-ethyl-2-(ethylthio)propanamide (Compound No. 1-001)

236 mg of 2-(ethylthio)propanoic acid in 4 ml of dichloromethane was mixed with 300 mg of oxalyl chloride and 10 mg of N,N-dimethylformamide successively and stirred at room temperature for 1 hour. After the stirring, the reaction solution was added dropwise to a separately prepared solution of 280 mg of 1-tert-butyl-5-chloro-N-ethyl-1 H-pyrazole-4-amine hydrochloride and 140 mg of pyridine in 4 ml of dichloromethane, and the reaction was stirred at room temperature for 30 minutes. After the reaction, the reaction solution was washed with 5 ml of water. The resulting organic layer was washed with saturated aqueous sodium chloride and dried over anhydrous sodium sulfate, and the solvent was evaporated under reduced pressure. The resulting residue was purified by preparative medium pressure liquid chromatography using n-hexane-ethyl acetate (4:1) as the eluent to obtain 300 mg of the desired product as a colorless oil.

¹H NMR (CDCl₃, Me₄Si, 300 MHz) δ7.48 (brs, 1H), 3.90-3.50 (m, 2H), 3.30-3.20 (m, 1H), 2.52 (q, J=7.2 Hz, 2H), 1.72 (s, 9H), 1.42 (d, J=7.2 Hz, 3H), 1.14 (t, J=7.2 Hz, 3H), 1.10 (t, J=7.2 Hz, 3H)

### Step 6

### Preparation of N-(3-chloro-1H-pyrazol-4-yl)-N-ethyl-2-(ethylthio)propanamide (Compound No. J-001)

0.2 g of N-(1-tert-butyl-5-chloro-1H-pyrazol-4-yl)-N-ethyl-2-(ethylthio)propanamide was mixed with 2 ml of concentrated hydrochloric acid under cooling with ice and stirred at room temperature overnight. After the reaction, the reaction mixture was cooled to 0° C., neutralized with aqueous sodium hydroxide and extracted with 10 ml of chloroform. The resulting organic layer was dried over anhydrous sodium sulfate, and the solvent was evaporated under reduced pressure to obtain 0.1 g of the desired product as a white solid.

m. p.; 78-80° C.

### Step 7

### Preparation of N-{3-chloro-1-(pyridin-3-yl)-1H-pyrazol-4-yl}-N-ethyl-2-(ethylthio)propanamide

To 100 mg of N-(3-chloro-1H-pyrazol-4-yl)-N-ethyl-2-(ethylthio)propanamide in 2 ml of N,N-dimethylformamide, 72 mg of 3-bromopyridine, 11 mg of copper (I) iodide, 215 mg of cesium carbonate and 16 mg of trans-N,N′-dimethylcyclohexane-1,2-diamine were added successively. After the addition, the atmosphere in the reaction vessel was replaced by nitrogen gas, and the reaction mixture was stirred at 140° C. for 20 hours. After the reaction, the reaction mixture was mixed with 20 ml of ethyl acetate, and the solid precipitated in the reaction mixture was filtered off. The filtrate was washed with 10 ml of water and then with saturated aqueous sodium chloride and dried over anhydrous sodium sulfate, and the solvent was evaporated under reduced pressure. The resulting residue was purified by silica gel column chromatography using n-hexane-ethyl acetate (with a gradient of from 7:3 to 3:7) as the eluent to obtain 21 mg of the desired product as a white solid.

m. p.; 65-66° C.

### Synthetic Example 33

### Preparation of N-{3-chloro-1-(pyridin-3-yl)-1H-pyrazol-4-yl}-2-(ethylthio)propanethioamide (Compound No. 1-353 of the present invention)

800 mg of N-{3-chloro-1-(pyridin-3-yl)-1H-pyrazol-4-yl}-2-(ethylthio)propanamide in 12 ml of toluene was mixed with 1.04 g of Lawesson's Reagent and refluxed with toluene for 3 hours with stirring. After the reaction, the reaction mixture was allowed to cool to room temperature, mixed with 15 ml of saturated aqueous sodium hydrogen carbonate and extracted with 40 ml of ethyl acetate. The resulting organic layer was with saturated aqueous sodium chloride and dried over anhydrous sodium sulfate, and the solvent was evaporated under reduced pressure. The resulting residue was purified by preparative medium pressure liquid chromatography using n-hexane-ethyl acetate (with a gradient of from 9:1 to 1:1) as the eluent to obtain 598 mg of the desired product as a yellow oil.

¹H NMR (CDCl₃, Me₄Si, 300 MHz) δ10.61 (brs, 1H), 9.52 (s, 1H), 8.99 (d, J=2.7 Hz, 1H), 8.58 (dd, J=4.8, 1.2 Hz, 1H), 8.01 (ddd, J=8.4, 2.7, 1.2 Hz, 1H), 7.42 (dd, J=8.4, 4.8 Hz, 1H), 4.21 (q, J=7.2 Hz, 1H), 2.67-2.49 (m, 2H), 1.64 (d, J=7.2 Hz, 3H), 1.29 (t, J=7.5 Hz, 3H)

### Synthetic Example 34

### Preparation of methyl N-3-chloro-1-(pyridin-3-yl)-1H-pyrazol-4-yl-2-(ethylthio)propanimidothioate (Compound No. 3-006 of the present invention)

540 mg of N-{3-chloro-1-(pyridin-3-yl)-1H-pyrazol-4-yl)-2-(ethylthio)propanethioamide in 4.5 ml of acetonitrile was mixed with 230 mg of potassium carbonate and 350 mg of methyl iodide and stirred at 50° C. for 1 hour. After the reaction, the reaction mixture was mixed with 10 ml of water and extracted with 20 ml of ethyl acetate. The resulting organic layer was with saturated aqueous sodium chloride and dried over anhydrous sodium sulfate, and the solvent was evaporated under reduced pressure. The resulting residue was purified by preparative medium pressure liquid chromatography using n-hexane-ethyl acetate (with a gradient of from 9:1 to 1:1) as the eluent to obtain 391 mg of the desired product as a yellow oil. The desired product was a 2:1 mixture of two geometrical isomers.

The isomer contained in a ratio of 2

¹H NMR (CDCl₃, Me₄Si, 300 MHz) δ8.89 (d, J=2.1 Hz, 1H), 8.54 (dd, J=4.8, 1.2 Hz, 1H), 8.01-7.99 (m, 1H), 7.59 (s, 1H), 7.40 (dd, J=8.4, 4.8 Hz, 1H), 4.12 (q, J=7.2 Hz, 1H), 2.71-2.53 (m, 2H), 2.42 (s, 3H), 1.51 (d, J=7.2 Hz, 3H), 1.19 (t, J=7.2 Hz, 3H)

The isomer contained in a ratio of 1

¹H NMR (CDCl₃, Me₄Si, 300 MHz) δ8.92-8.89 (m, 1H), 8.54 (dd, J=4.8, 1.2 Hz, 1H), 8.01-7.99 (m, 1H), 7.93 (s, 1H), 7.40 (dd, J=8.4, 4.8 Hz, 1H), 3.89 (q, J=6.9 Hz, 1H), 2.71-2.53 (m, 2H), 2.65 (s, 3H), 1.69 (d, J=6.9 Hz, 3H), 1.27 (t, J=7.2 Hz, 3H)

### Synthetic Example 35

### Preparation of N-{3-chloro-1-(pyridin-3-yl)-1H-pyrazol-4-yl}-2-(ethylthio)-N′-methoxypropanimidamide (Compound No. 1-356 of the present invention)

284 mg of methyl N-3-chloro-1-(pyridin-3-yl)-1H-pyrazol-4-yl-2-(ethylthio)propanimidothioate in 4 ml of ethanol was mixed with 110 mg of O-methylhydroxylamine hydrochloride and 132 mg of triethylamine successively and stirred at 50° C. for 3 hours. After the stirring, 110 mg of O-methylhydroxylamine hydrochloride and 132 mg of triethylamine were added again, and the reaction solution was stirred at 50° C. for another 4 hours. After the stirring, 220 mg of O-methylhydroxylamine hydrochloride and 265 mg of triethylamine were further added, and the reaction solution was stirred at 50° C. for another 3 hours. After the reaction, the reaction solution was mixed with 10 ml of water and extracted with 20 ml of ethyl acetate. The resulting organic layer was with saturated aqueous sodium chloride and dried over anhydrous sodium sulfate, and the solvent was evaporated under reduced pressure. The resulting residue was purified by preparative medium pressure liquid chromatography using n-hexane-ethyl acetate (with a gradient of from 9:1 to 1:1) as the eluent to obtain 237 mg of the desired product as a yellow oil. The desired product was a 5:2 mixture of two isomers.

The isomer contained in a ratio of 5

¹H NMR (CDCl₃, Me₄Si, 300 MHz) δ8.93 (d, J=2.7 Hz, 1H), 8.52 (dd, J=5.1, 1.2 Hz, 1H), 8.51 (s, 1H), 8.00 (ddd, J=8.4, 2.7, 1.2 Hz, 1H), 7.39 (dd, J=8.4, 5.1 Hz, 1H), 6.92 (brs, 1H), 4.64 (q, J=7.2 Hz, 1H), 3.88 (s, 3H), 2.65-2.44 (m, 2H), 1.45 (d, J=7.2 Hz, 3H), 1.26 (t, J=7.5 Hz, 3H)

The isomer contained in a ratio of 2

¹H NMR (CDCl₃, Me₄Si, 300 MHz) δ8.92 (d, J=2.7 Hz, 1H), 8.58 (dd, J=4.5, 1.2 Hz, 1H), 8.08 (s, 1H), 8.00 (ddd, J=8.4, 2.7, 1.2 Hz, 1H), 7.57 (dd, J=8.4, 4.5 Hz, 1H), 6.38 (brs, 1H), 3.43 (q, J=7.2 Hz, 1H), 3.87 (s, 3H), 2.65-2.44 (m, 2H), 1.47 (d, J=7.2 Hz, 3H), 1.24 (t, J=7.5 Hz, 3H)

### Synthetic Example 36

### Preparation of N-{3-chloro-1-(pyridin-3-yl)-1H-pyrazol-4-yl}-2-(ethylthio)-N-(pyop-2-ynyl)propanamide (Compound No. 1-289 of the present invention)

0.15 g of 60 wt % sodium hydride (dispersed in mineral oil) in 5 ml of tetrahydrofuran was mixed with 0.48 g of N-{3-chloro-1-(pyridin-3-yl)-1H-pyrazol-4-yl)-2-(ethylthio)propanamide under cooling with ice and stirred for 30 minutes. After the stirring, the reaction mixture was mixed with 63 μL of about 9.2 mol/L propargyl bromide in toluene and stirred at room temperature for another 30 minutes. After the reaction, the reaction mixture was poured into 10 ml of ice-water and extracted with 10 ml of ethyl acetate. The resulting organic layer was with saturated aqueous sodium chloride and dried over anhydrous sodium sulfate, and the solvent was evaporated under reduced pressure. The resulting residue was purified by preparative medium pressure liquid chromatography using n-hexane-ethyl acetate (2:1) as the eluent to obtain 0.13 g of the desired product as a pale yellow amorphous substance.

¹H NMR (CDCl₃, Me₄Si, 300 MHz) δ8.95 (d, J=2.4 Hz, 1H), 8.62 (dd, J=4.8, 1.2 Hz, 1H), 8.22 (s, 1H), 8.04 (ddd, J=8.1, 2.4, 1.2 Hz, 1H), 7.45 (dd, J=8.1, 4.8 Hz, 1H), 4.97-4.60 (m, 1H), 4.30-3.95 (m, 1H), 3.39-3.27 (m, 1H), 2.66-2.52 (m, 2H), 2.25 (t, J=2.1 Hz, 1H), 1.48 (d, J=6.9 Hz, 3H), 1.17 (t, J=7.2 Hz, 3H)

### Synthetic Example 37

### Preparation of (S)-(−)-N-{3-chloro-1-(pyridin-3-yl)-1H-pyrazol-4-yl}-N-ethyl-2-methyl-3-(methylthio)propanamide [Compound No. 1-048(S)-(−) of the present invention]

To a solution of 0.905 g of (S)-2-methyl-3-(methylthio)propanoic acid synthesized in accordance with US-A-2005-146823 in 20 ml of dichloromethane, 1.14 g of oxalyl chloride and 10 mg of N,N-dimethylformamide were added successively, and the reaction mixture was stirred at room temperature for 2 hours. After the reaction, the solvent was evaporated under reduced pressure, and the resulting residue was mixed with 1 g of 3-chloro-N-ethyl-1-(pyridin-3-yl)-1H-pyrazole-4-amine and 1.07 g of pyridine in 20 ml of dichloromethane and stirred at room temperature for 1 hour. After the reaction, the reaction solution was washed with 20 ml of water. The resulting organic layer was washed with saturated aqueous sodium chloride and dried over anhydrous sodium sulfate, and the solvent was evaporated under reduced pressure. The resulting residue was purified by preparative medium pressure liquid chromatography using n-hexane-ethyl acetate (1:1) as the eluent and recrystallized from a solvent mixture of n-hexane and ethyl acetate (5:1) to obtain 0.4 g of the desired product as a white solid. The desired product was optically resolved by high performance liquid chromatography using an optically active column under the conditions described later, and 99% of it, in terms of peak area percentage, was detected as a fraction with a retention time of 18.4 min {[α]_{D}^{23.9}−10.00° (CHCl₃, c=0.510)}.

### REFERENCE EXAMPLES

### Reference Example 1

The following intermediates were synthesized in the same manner as in Step 1 in Synthetic Example 3.
A-002; N-ethyl-3-methyl-1-(pyridin-3-yl)-1H-pyrazole-4-amine dihydrochloride

m. p.; 113-116° C.
A-005; N-allyl-3-chloro-1-(pyridin-3-yl)-1H-pyrazole-4-amine

¹H NMR (D₂O, Me₄Si, 300 MHz) δ9.05 (d, J=2.7 Hz, 1H), 8.66 (ddd, J=8.4, 2.4, 1.2 Hz, 1H), 8.59 (ddd, J=5.7, 0.9, 0.6 Hz, 1H), 8.32 (brs, 1H), 8.02 (ddd, J=9.3, 5.7, 0.6 Hz, 1H), 5.33-5.25 (m, 1H), 5.89-5.76 (m, 2H), 3.79 (d, J=6.3 Hz, 2H) (no detectable peaks for NH)
A-009; 3-methyl-1-(pyridin-3-yl)-1H-pyrazole-4-amine dihydrochloride

m. p.; 198-206° C.
A-010; 1-(pyridin-3-yl)-1H-pyrazole-4-amine dihydrochloride

m. p.; 195-196° C. (decomposition)

### Reference Example 2

The following intermediates were synthesized in the same manner as in Step 1 in Synthetic Example 25.
B-001; N,3-dimethyl1-(pyridin-3-yl)-1H-pyrazole-4-amine

m. p.; 74-76° C.
B-002; N-ethyl-3-methyl-1-(pyridin-3-yl)-1H-pyrazole-4-amine

m. p.; 80-81° C.
B-006; 3-chloro-N-n-propyl-1-(pyridin-3-yl)-1H-pyrazole-4-amine

¹H NMR (CDCl₃, Me₄Si, 300 MHz) δ8.86 (d, J=2.7 Hz, 1H), 8.46 (d, J=3.6 Hz, 1H), 7.94 (ddd, J=9.6, 2.7, 1.2 Hz, 1H), 7.37-7.32 (m, 2H), 3.15 (brs, 1H), 3.02 (t, J=7.2 Hz, 2H), 1.69 (q, J=7.2 Hz, 2H), 1.02 (t, J=7.5 Hz, 3H)
B-007; 3-chloro-N-isopropyl-1-(pyridin-3-yl)-1H-pyrazole-4-amine

¹H NMR (CDCl₃, Me₄Si, 300 MHz) δ8.87 (d, J=2.7 Hz, 1H), 8.48 (dd, J=4.8, 1.5 Hz, 1H), 7.96 (ddd, J=8.4, 2.7, 1.5 Hz, 1H), 7.38-7.34 (m, 2H), 3.34 (qq, J=4.8, 4.8 Hz, 1H), 2.85 (brs, 1H), 1.25 (d, J=6.6 Hz, 6H)
B-008; 3-chloro-N-(prop-2-ynyl)-1-(pyridin-3-yl)-1H-pyrazole-4-amine

m. p.; 123-124° C.
B-009; 3-methyl-1-(pyridin-3-yl)-1H-pyrazole-4-amine

m. p.; 120-122° C.
B-010; 3-bromo-N-ethyl-1-(pyridin-3-yl)-1H-pyrazole-4-amine

m. p.; 109-110
B-011; 1-(pyridin-3-yl)-1H-pyrazole-4-amine

m. p.; 88-89° C.
B-012; 3-chloro-1-(pyridin-3-yl)-N-(2,2,2-trifluoroethyl)-1H-pyrazole-4-amine

m. p.; 79-82° C.
B-013; N-ethyl-5-methyl-1-(pyridin-3-yl)-1H-pyrazole-4-amine

¹H NMR (CDCl₃, Me₄Si, 300 MHz) δ8.76-8.70 (m, 1H), 8.57 (dd, J=4.8, 1.2 Hz, 1H), 7.81 (ddd, J=8.1, 2.4, 1.2 Hz, 1H), 7.44-7.36 (m, 1H), 7.42 (s, 1H), 3.11 (q, J=7.2 Hz, 2H), 2.26 (s, 3H), 1.25 (t, J=7.2 Hz, 3H) (no detectable peaks for NH)
B-014; 3-chloro-N-(cyclopropylmethyl)-1-(pyridin-3-yl)-1H-pyrazole-4-amine

m. p.; 72-78° C.
B-015; 3-chloro-N-(2-methoxyethyl)-1-(pyridin-3-yl)-1H-pyrazole-4-amine

m. p.; 68-69° C.
B-016; 3-chloro-1-(pyridin-3-yl)-N-{(trimethylsilyl)methyl}-1H-pyrazole-4-amine

m. p.; 105-111° C.
B-017; N-ethyl-3-methoxy-1-(pyridin-3-yl)-1H-pyrazole-4-amine

¹H NMR (CDC₃, Me₄Si, 300 MHz) δ8.79 (d, J=2.7 Hz, 1H), 8.31 (dd, J=4.8, 1.2 Hz, 1H), 7.81 (ddd, J=8.4, 2.7, 1.2 Hz, 1H), 7.27 (dd, J=8.4, 4.8 Hz, 1H), 7.21 (s, 1H), 4.01 (s, 3H), 3.05 (q, J=7.2 Hz, 2H), 2.63 (brs, 1H), 1.27 (t, J=7.2 Hz, 3H)
B-018; N-ethyl-1-(pyridin-3-yl)-3-(trifluoromethyl)-1H-pyrazole-4-amine

m. p.; 88-89° C.
B-020; N-ethyl-3-(methylsulfonyl)-1-(pyridin-3-yl)-1H-pyrazole-4-amine

m. p.; 139-141° C.
B-020; 3-bromo-N-methyl-1-(pyridin-3-yl)-1H-pyrazole-4-amine

m. p.; 107-109° C.
B-021; 3-chloro-1-(pyridin-3-yl)-1H-pyrazole-4-amine

m. p.; 70-72° C.
B-022; 3-bromo-1-(pyridin-3-yl)-1H-pyrazole-4-amine

m. p.; 138-140° C.

### Reference Example 3

The following intermediate was synthesized in the same manner as in Step 1 in Synthetic Example 28.
C-002; 3-chloro-N-{2-(methylthio)ethyl}-1-(pyridin-3-yl)-1H-pyrazole-4-amine

m. p.; 66-68° C.

### Reference Example 4

The following intermediates were synthesized in the same manner as in Step 1 in Synthetic Example 20.
D-002; ethyl 2-(ethoxyimino)-3-(methylthio)propanoate

¹H NMR (CDCl₃, Me₄Si, 300 MHz) δ4.40-4.25 (m, 4H), 3.57 (s, 2H), 2.11 (s, 3H), 1.38 (t, J=7.1 Hz, 3H), 1.31 (t, J=6.3 Hz, 3H)
D-003; ethyl 2-(isopropoxyimino)-3-(methylthio)propanoate

¹H NMR (CDCl₃, Me₄Si, 300 MHz) δ4.56 (sep, J=6.3 Hz, 1H), 4.34 (q, J=7.1 Hz, 2H), 3.57 (s, 2H), 2.10 (s, 3H), 1.35 (t, J=7.1 Hz, 3H), 1.30 (d, J=6.3 Hz, 6H)
D-004; ethyl 3-(methylthio)-2-(n-propoxyimino)propanoate

¹H NMR (CDCl₃, Me₄Si, 300 MHz) δ4.35 (q, J=7.1 Hz, 2H), 4.22 (t, J=7.1 Hz, 2H), 3.58 (s, 2H), 2.11 (s, 3H), 1.72 (m, 2H), 1.35 (t, J=7.1 Hz, 3H), 0.96 (t, J=7.1 Hz, 3H)
D-005; ethyl 2-(benzyloxyimino)-3-(methylthio)propanoate

¹H NMR (CDCl₃, Me₄Si, 300 MHz) δ7.40-7.35 (m, 5H), 5.28 (s, 2H), 4.33 (q, J=7.1 Hz, 2H), 3.58 (s, 2H), 2.05 (s, 3H), 1.35 (t, J=7.1 Hz, 3H)
D-006; ethyl 2-(butoxyimino)-3-(methylthio)propanoate

¹H NMR (CDCl₃, Me₄Si, 300 MHz) δ4.35 (q, J=7.1 Hz, 2H), 4.27 (t, J=6.5 Hz, 2H), 3.57 (s, 2H), 2.13 (s, 3H), 1.69 (tt, J=7.1, 6.5 Hz, 2H), 1.39 (sxt, J=7.1 Hz, 2H), 1.35 (t, J=7.1 Hz, 3H), 0.94 (t, J=7.1 Hz, 3H)
D-007; ethyl 2-(methoxyimino)-3-(2,2,2-trifluoroethylthio)propanoate

¹H NMR (CDCl₃, Me₄Si, 300 MHz) δ4.37 (q, J=7.1 Hz, 2H), 4.09 (s, 3H), 3.69 (s, 2H), 3.15 (q, J=9.9 Hz, 2H), 1.36 (t, J=7.1 Hz, 3H)
D-008; ethyl (2-methoxyethoxyimino)-3-(methylthio)propanoate

¹H NMR (CDCl₃, Me₄Si, 300 MHz) δ4.40 (t, J=4.8 Hz, 2H), 4.34 (q, J=7.1 Hz, 2H), 3.66 (t, J=4.8 Hz, 2H), 3.60 (s, 2H), 3.37 (s, 3H), 2.11 (s, 3H), 1.35 (t, J=7.1 Hz, 3H)
D-009; ethyl 2-(allyloxyimino)-3-(methylthio)propanoate

¹H NMR (CDCl₃, Me₄Si, 300 MHz) δ5.98 (ddt, J=17.1, 10.5, 5.7 Hz, 1H), 5.33 (dd, J=17.1, 1.5 Hz, 1H), 5.26 (dd, J=10.5, 1.5 Hz, 1H), 4.76 (d, J=5.7 Hz, 2H), 4.34 (q, J=7.1 Hz, 2H), 3.59 (s, 2H), 2.11 (s, 3H), 1.34 (t, J=7.1 Hz, 3H)
D-010; ethyl 2-(methoxyimino)-3-(trifluoromethylsulfonyl)propanoate

¹H NMR (CDCl₃, Me₄Si, 300 MHz) δ4.64 (s, 2H), 4.38 (q, J=7.2 Hz, 2H), 4.19 (s, 3H), 1.36 (t, J=7.2 Hz, 3H)

### Reference Example 5

The following intermediates were synthesized in the same manner as in Step 2 in Synthetic Example 20.
E-002; 2-(ethoxyimino)-3-(methylthio)propanoic acid

¹H NMR (CDCl₃, Me₄Si, 300 MHz) δ4.34 (q, J=7.1 Hz, 2H), 3.54 (s, 2H), 2.14 (s, 3H), 1.34 (t, J=7.1 Hz, 3H) (no detectable peaks for CO₂H)
E-003; 2-(isopropoxyimino)-3-(methylthio)propanoic acid

¹H NMR (CDCl₃, Me₄Si, 300 MHz) δ4.54 (sep, J=6.5 Hz, 1H), 3.53 (s, 2H), 2.12 (s, 3H), 1.31 (d, J=6.5 Hz, 6H) (no detectable peaks for CO₂H)
E-004; 3-(methylthio)-2-(n-propoxyimino)propanoic acid

¹H NMR (CDCl₃, Me₄Si, 300 MHz) δ4.22 (t, J=7.1 Hz, 2H), 3.54 (s, 2H), 2.13 (s, 3H), 1.74 (m, 2H), 0.96 (t, J=7.1 Hz, 3H)(no detectable peaks for CO₂H)
E-005; 2-(benzyloxyimino)-3-(methylthio)propanoic acid

¹H NMR (CDCl₃, Me₄Si, 300 MHz) δ7.40-7.30 (m, 5H), 5.23 (s, 2H), 3.47 (s, 2H), 1.91 (s, 3H) (no detectable peaks for CO₂H)
E-006; 2-(butoxyimino)-3-(methylthio)propanoic acid

¹H NMR (CDCl₃, Me₄Si, 300 MHz) δ4.10 (t, J=6.5 Hz, 2H), 3.47 (s, 2H), 2.01 (s, 3H), 1.57 (tt, J=7.1, 6.5 Hz, 2H), 1.35 (sxt, J=7.1 Hz, 2H), 0.90 (t, J=7.1 Hz, 3H) (no detectable peaks for CO₂H)
E-007; 2-(methoxyimino)-3-(2,2,2-trifluoroethylthio)propanoic acid

¹H NMR (CDCl₃, Me₄Si, 300 MHz) δ3.90 (s, 3H), 3.57 (s, 2H), 3.12 (q, J=9.9 Hz, 2H) (no detectable peaks for CO₂H)
E-008; (2-methoxyethoxyimino)-3-(methylthio)propanoic acid

¹H NMR (CDCl₃, Me₄Si, 300 MHz) δ4.43 (t, J=4.8 Hz, 2H), 3.68 (t, J=4.8 Hz, 2H), 3.57 (s, 2H), 3.39 (s, 3H), 2.15 (s, 3H) (no detectable peaks for CO₂H)
E-009; 2-(allyloxyimino)-3-(methylthio)propanoic acid

¹H NMR (CDCl₃, Me₄Si, 300 MHz) δ5.95 (ddt, J=17.1, 10.5, 5.7 Hz, 1H), 5.40-5.30 (m, 1H), 5.30-5.20 (m, 1H), 4.73 (d, J=5.7 Hz, 2H), 3.54 (s, 2H), 2.13 (s, 3H) (no detectable peaks for CO₂H)
E-010; 2-(methoxyimino)-3-(trifluoromethylsulfonyl)propanoic acid

¹H NMR (CDCl₃, Me₄Si, 300 MHz) δ4.64 (s, 2H), 4.26 (s, 3H) (no detectable peaks for CO₂H)

### Reference Example 6

### Synthesis of methyl 4-amino-1-(pyridin-3-yl)-1H-pyrazole-3-carboxylate

3-Bromopyridine and methyl 4-amino-1H-pyrazole-3-carboxylate were reacted in the same manner as in Step 7 in Synthetic Example 32 to give methyl 4-amino-1-(pyridin-3-yl)-1H-pyrazole-3-carboxylate.

m. p.; 102-108° C.

### Reference Example 7

The following intermediates were synthesized in the same manner as in Step 3 in Synthetic Example 32.

G-002; tert-butyl 1-tert-butyl-5-chloro-1H-pyrazol-4-yl(methyl)carbamate

m. p.; 30-31° C.

### Reference Example 8

The following intermediates were synthesized in the same manner as in Step 4 in Synthetic Example 32.
H-002; 1-tert-butyl-5-chloro-N-methyl-1H-pyrazole-4-amine

¹H NMR (CDCl₃, Me₄Si, 300 MHz) δ7.14 (s, 1H), 2.81 (s, 3H), 1.66 (s, 9H) (no detectable peaks for NH)

### Reference Example 9

The following intermediates were synthesized in the same manner as in Step 5 in Synthetic Example 32.
I-002; N-(1-tert-butyl-5-chloro-1H-pyrazol-4-yl)-N,2-dimethyl-3-(methylthio)propanamide

m. p.; 41-42° C.
I-003; N-(1-tert-butyl-5-chloro-1H-pyrazol-4-yl)-N-ethyl-2-methyl-3-(methylthio)propanamide

m. p.; 52-53° C.

### Reference Example 10

The following intermediates were synthesized in the same manner as in Step 6 in Synthetic Example 32.
J-002; N-(3-chloro-1H-pyrazol-4-yl)-N,2-dimethyl-3-(methylthio)propanamide

m. p.; 91-93° C.
J-003; N-(3-chloro-1H-pyrazol-4-yl)-N-ethyl-2-methyl-3-(methylthio)propanamide

m. p.; 111-112° C.

### Reference Example 11

### Synthesis of 3-chloro-1-(pyridin-3-yl)-1H-pyrazole-4-amine (Compound No. B-021)

3-Bromopyridine and 3-chloro-1H-pyrazole-4-amine were reacted in the same manner as in Step 7 in Synthetic Example 32 to give 3-chloro-1-(pyridin-3-yl)-1H-pyrazole-4-amine.

m. p.; 70-72° C.

The compounds of the present invention can be produced in accordance with the processes and Examples previously described. Examples of the compounds of the present invention in the same manners as in Synthetic Examples 1 to 37 are recited in Tables 4 to 10 and their physical properties are shown in Tables 11 and 12. However, the present invention is by no means restricted thereto. In the Tables, D1-1a, D1-1b, D1-2a, D1-2c, D1-4-a, D1-5e, D1-5f, D1-6d, D1-7b, D1-8c, D1-8f, D1-9f, D1-10e, D1-10f, D1-12a, D1-12f, D1-13d, D1-19a, D1-28a, D1-32a, D1-32b, D1-33a, D1-33b, D1-34a, D1-35b, D1-37a, D1-37b, D1-38b, D1-45d, D1-51a, D1-80a, D1-81a, D1-81b, D1-82a, D1-82b, D1-82c, D1-84d, D1-85d, D1-87a, D1-88a, D1-88b, D1-92c, D1-93a, D1-94b, D1-94c, D1-98a, D1-103b, D1-103c, D1-103d, D1-103e, D1-103f, D1-103g, D1-103h, D1-103i, D1-108a and D1-108b represent the following structures, and the numbers in the structural formulae D1-1b, D1-2c, D1-5e, D1-5f, D1-6d, D1-7b, D1-8f, D1-9f, D1-10e, D1-10f, D1-12e, D1-12f, D1-32b, D1-33b, D1-37b and D1-38b indicate the positions of the substituent X¹, the numbers in the structural formula D1-45d indicate the positions of the substituent X^{1b}, and the numbers in the structural formula D1-108b indicate the positions of the substituent Z. The numbers in the structural formulae in Table 10 indicate the positions of the substituent R².

*1 means resinous. *2 means a resinous mixture of two isomers having the same structure as in Synthetic Example 10, and, if known, the ratio of the isomers is shown in Table 11. *3 means the melting point of a mixture of two compounds having different structures, as obtained in Synthetic Example 8. *4 means the geometrical isomer of the oxime 1-170.

The symbols in Tables 11 and 12 have the following meanings.

s: singlet, d: doublet, t: triplet, q: quartet, qui: quintet, sxt: sextet, sep: septet, m: multiplet, brs: broad peak.

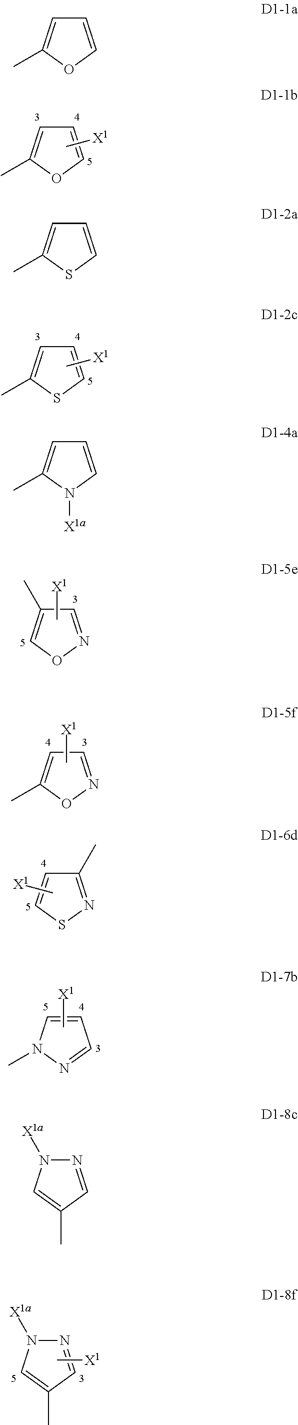

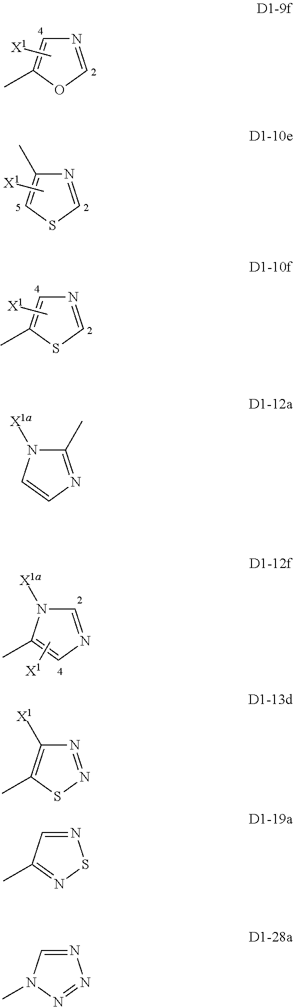

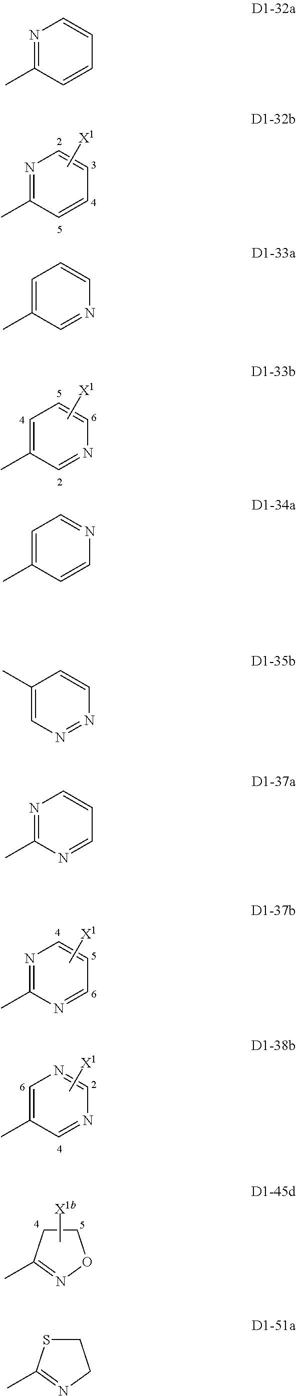

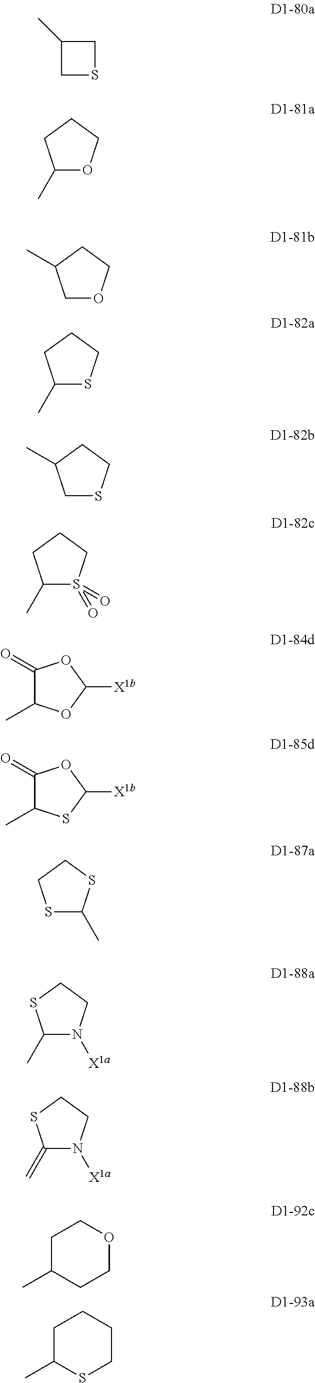

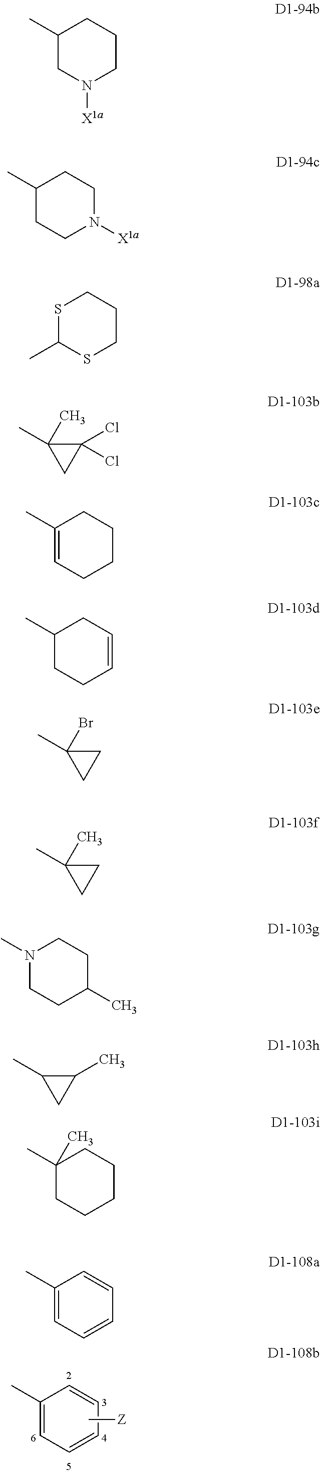

TABLE 4

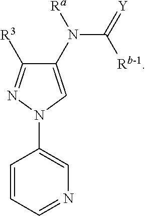

| No. | R³ | Y | R^{a} | R^{b-1} | m.p. (° C.) |
|---|---|---|---|---|---|
| 1-001 | H | O | H | CH₂CF₃ | 178-183 |
| 1-002 | H | O | H | CH₂S(O)₂CH₃ | 202-205 |
| 1-003 | H | O | H | CH₂SCH₃ | 173-176 |
| 1-004 | H | O | H | NHCH₂CF₃ | *1 |
| 1-005 | H | O | H | NHCH₂CH₃ | 145-151 |
| 1-006 | H | O | H | CH₂CH₂SCH₃ | 134-138 |
| 1-007 | H | O | H | CH═C(CH₃)CF₃ | 210-216 |
| 1-008 | H | O | H | CH═CHCF₃ | 248-249 |
| 1-009 | H | O | H | CH═CH{D1-108b(4-CF₃)} | *1 |
| 1-010 | H | NCN | H | NHCH₂CH₃ | 190-197 |
| 1-011 | Me | O | H | OC(CH₃)₃ | 112-116 |
| 1-012 | Me | O | CH₃ | OC(CH₃)₃ | 65-70 |
| 1-013 | Me | O | CH₂CH₃ | OC(CH₃)₃ | 73-76 |
| 1-014 | H | O | H | CH₂CH(OH)CF₃ | 157-158 |
| 1-015 | H | NCN | H | NHCH₂CF₃ | 205-210 |
| 1-016 | Me | NCN | H | NHCH₂CH₃ | 170-175 |
| 1-017 | Me | NCN | H | NHCH₂CF₃ | 215-223 |
| 1-018 | Me | O | CH₃ | CH₂CH₂SCH₃ | *1 |
| 1-019 | Me | O | CH₂CH₃ | CH₂CH₂SCH₃ | *1 |
| 1-020 | H | O | H | OC(CH₃)₃ | 110-115 |
| 1-021 | H | O | CH₃ | OC(CH₃)₃ | *1 |
| 1-022 | H | O | CH₂CH₃ | OC(CH₃)₃ | *1 |
| 1-023 | Me | O | CH₃ | CH(CH₃)CH₂SCH₃ | *1 |
| 1-024 | H | O | H | D1-8c(CH₂CF₃) | 145-155 |
| 1-025 | H | O | H | CH(Br)CH₂CH₂CH₂Br | 114-115 |
| 1-026 | H | O | —CH₂CH₂CH₂— | | *1 |
| 1-027 | H | O | —CH₂CH₂CH₂CH(Br)— | | 120-121 |
| 1-028 | H | O | —CH₂CH₂CH₂CH(SCH₃)— | | 107-108 |
| 1-029 | H | O | —CH₂OCH(CF₃)CH₂— | | 147-148 |
| 1-030 | H | O | —CH₂OCH₂N(CH₂CF₃)— | | 138-141 |
| 1-031 | Me | O | CH₂CH₃ | CH(CH₃)CH₂SCH₃ | *1 |
| 1-031(S)-(+) | | 99% e.e. | [α]_{D}^{24.4} + 32.20° | (CHCl₃, c = 0.682) | *1 |
| 1-032 | Me | O | H | NHCH₂CF₃ | 228-230 |
| 1-033 | Cl | O | CH₂CH₃ | CH₂CH₂SCH₃ | *1 |
| 1-034 | H | O | —CH₂CH₂CH₂CH₂— | | 123-124 |
| 1-035 | H | O | —CH₂CH₂CH₂CH{S(O)₂CH₃}— | | 163-164 |
| 1-036 | H | O | —CH₂CH₂CH₂C(F){S(O)₂CH₃}— | | 173-174 |
| 1-037 | Cl | O | CH₃ | OC(CH₃)₃ | 83-84 |
| 1-038 | Cl | O | CH₃ | CH₂CH₂SCH₃ | *1 |
| 1-039 | Cl | O | CH₃ | CH(CH₃)CH₂SCH₃ | *1 |
| 1-039(S)-(−) | | 99% e.e. | [α]_{D}^{24.4} − 10.76° | (CHCl₃,c = 0.502) | 76-77 |
| 1-040 | Cl | O | CH₂CH₃ | CH₂CH₂S(O)CH₃ | *1 |
| 1-041 | Cl | O | CH₂CH₃ | CH₂CH₂S(O)₂CH₃ | 103-108 |
| 1-042 | H | O | CH₃ | CH₂CH₂SCH₃ | *2(~3:2) |
| 1-043 | H | O | CH₃ | CH(CH₃)CH₂SCH₃ | *2(~3:1) |
| 1-044 | H | O | CH₂CH₃ | CH₂CH₂SCH₃ | *2(~3:1) |
| 1-045 | H | O | CH₂CH₃ | CH(CH₃)CH₂SCH₃ | *2(~5:1) |
| 1-046 | Cl | O | H | OC(CH₃)₃ | 125-128 |
| 1-047 | H | O | (—CH₂CH₂CH═CH—) & (—CH₂CH═CHCH₂—) | | 128-129(*3) |
| 1-048 | Cl | O | CH₂CH₃ | CH(CH₃)CH₂SCH₃ | 64-65 |
| 1-048(R)-(+) | | 84% e.e. | [α]_{D}^{24.1} + 8.95° | (CHCl₃,c = 0.503) | 64-65 |
| 1-048(S)-(−) | | 99% e.e. | [α]_{D}^{23.9} − 10.00° | (CHCl₃,c = 0.510) | 64-65 |
| 1-049 | Cl | O | CH₂CH₃ | CH(CH₃)CH₂S(O)CH₃ | *2(~10:7) |
| 1-050 | Cl | O | CH₂CH₃ | CH(CH₃)CH₂S(O)₂CH₃ | *1 |
| 1-050(S)-(+) | | 99% e.e. | [α]_{D}^{24.4} + 23.20° | (CHCl₃,c = 0.682) | *1 |
| 1-051 | Cl | O | CH₃ | CH₂CH₂S(O)CH₃ | *1 |
| 1-052 | Cl | O | CH₃ | CH₂CH₂S(O)₂CH₃ | 90-91 |
| 1-053 | Cl | O | CH₃ | CH(CH₃)CH₂S(O)CH₃ | *2(~4:1) |
| 1-054 | Cl | O | CH₃ | CH(CH₃)CH₂S(O)₂CH₃ | 153-154 |
| 1-054(S)-(+) | | 96% e.e. | [α]_{D}^{24.4} + 23.20° | (CHCl₃,c = 0.500) | 115-116 |
| 1-055 | H | O | H | CH₂CH₂CH₂Cl | 131-132 |
| 1-056 | Cl | O | CH₂CH₃ | OC(CH₃)₃ | 55-58 |
| 1-057 | H | O | CH₃ | CH₂S(O)₂CH₃ | 152-159 |
| 1-058 | Cl | O | H | CH₂CH₂SCH₃ | 105-115 |
| 1-059 | Me | O | CH₂CH₃ | O{D1-108b(4-NO₂)} | *1 |
| 1-060 | Me | O | CH₂CH₃ | NHCH₂CH₂SCH₃ | *1 |
| 1-061 | Me | O | CH₃ | NHCH₂CH₂SCH₃ | 82-86 |
| 1-062 | Me | O | H | NHCH₂CH₂SCH₃ | 145-147 |
| 1-063 | Cl | O | CH₂CH₂CH₃ | CH₂CH₂SCH₃ | *1 |
| 1-064 | Cl | O | CH₂CH═CH₂ | CH₂CH₂SCH₃ | *1 |
| 1-065 | Cl | O | CH₂CH₃ | CH₂CH(CH₃)SCH₃ | *1 |
| 1-066 | Cl | O | CH₂CH₂CH₃ | CH(CH₃)CH₂SCH₃ | *1 |
| 1-067 | Cl | O | CH₂CH₃ | CH₂OCH₂SCH₃ | *1 |
| 1-068 | Cl | O | CH₂OCH₃ | CH₂CH₂SCH₃ | *1 |
| 1-069 | Me | O | CH₃ | NHCH(CH₃)₂ | 130-132 |
| 1-070 | Me | S | CH₃ | NHCH(CH₃)₂ | 148-150 |
| 1-071 | Cl | O | CH₂CH₃ | CH═CHSCH₃ | 117-118 |
| 1-072 | Me | O | H | NHCH₂CH₂CH₂SCH₃ | 60-70 |
| 1-073 | Cl | O | CH₂CH₃ | CH₂CH(SCH₃)₂ | 76-77 |
| 1-074 | Cl | O | H | CH(CH₃)CH₂SCH₃ | 100-110 |
| 1-075 | Cl | O | CH(CH₃)₂ | CH(CH₃)CH₂SCH₃ | 110-111 |
| 1-076 | Cl | O | CH₂CH═CH₂ | CH(CH₃)CH₂SCH₃ | *1 |
| 1-077 | Cl | O | CH₂CH₃ | CH₂CH₂SC(O)CH₃ | *1 |
| 1-078 | Cl | O | CH(CH₃)₂ | CH₂CH₂SCH₃ | *1 |
| 1-079 | Cl | O | CH₂CH₃ | CH₂SCH₃ | *1 |
| 1-080 | Cl | O | CH₂CH₃ | CH₂S(O)₂CH₃ | 107-108 |
| 1-081 | Me | O | CH₃ | NHCH₂C(O)OCH₃ | 129-132 |
| 1-082 | Cl | O | CH₂CH₃ | CH₂OC(O)CH₃ | 114-116 |
| 1-083 | Cl | O | CH₂CH₃ | D1-82b | *1 |
| 1-084 | Cl | O | CH₃ | CH(CH₂CH₃)CH₂SCH₃ | 62-63 |
| 1-085 | Cl | O | CH₂CH₃ | CH₂CH₂SCH₂CH₃ | *1 |
| 1-086 | Cl | O | CH₂OCH₃ | CH(CH₃)CH₂SCH₃ | *1 |
| 1-087 | Me | O | CH₃ | CH₂CH₂S(O)CH₃ | *1 |
| 1-088 | Me | O | CH₃ | CH₂CH₂S(O)₂CH₃ | *1 |
| 1-089 | Me | O | CH₃ | NHC(O)OCH₂CH₃ | 144-147 |
| 1-090 | Me | O | CH₃ | NHC(Cl)₃ | 110-112 |
| 1-091 | Me | O | CH₃ | NHS(O)₂(D1-108a) | 130-133 |
| 1-092 | Cl | O | CH₂CH₃ | C(O)CH₃ | 73-77 |
| 1-093 | Cl | O | {—C(O)CH═C(CH₃)—} & {—C(O)CH₂C(═CH₂)—} | | 115-125(*3) |
| 1-094 | Me | O | CH₃ | NH₂ | 169-171 |
| 1-095 | Me | O | CH₃ | NHC(O)CH₂SCH₃ | *1 |
| 1-096 | Cl | O | CH₂CH₃ | CH₂S(O)CH₂CH₃ | 145-147 |
| 1-097 | Cl | O | CH₂CH₃ | CH₂S(O)₂CH₂CH₃ | *1 |
| 1-098 | Cl | O | CH₂CH₃ | SCH₃ | *1 |
| 1-099 | Cl | O | CH₂CH₃ | CH(CH₃)₂ | 107-111 |
| 1-100 | Cl | O | CH₂CH₃ | CH₂S(O)CH₃ | 115-116 |
| 1-101 | Cl | O | CH₂CH₃ | CH(CH₃)SCH₃ | 94-96 |
| 1-102 | Cl | O | CH₃ | CH₂SCH₃ | *1 |
| 1-103 | Cl | S | CH₂CH₃ | CH(CH₃)CH₂SCH₃ | *1 |
| 1-104 | Cl | O | CH₂CH₂CH₃ | CH₂CH₂S(O)CH₃ | *1 |
| 1-105 | Cl | O | CH₂CH₂CH₃ | CH₂CH₂S(O)₂CH₃ | *1 |
| 1-106 | Me | O | CH₂CH₃ | CH₂SCH₃ | *1 |
| 1-107 | Cl | O | CH₂CH₃ | CH₂SCH₂CH₃ | *1 |
| 1-108 | Cl | O | CH₂CH₃ | CH₂Br | 220-222 |
| 1-109 | Cl | O | CH₂CH₃ | CH₂SCH₂CH═CH₂ | *1 |
| 1-110 | Me | O | CH₂CH₃ | CH₂CH₂S(O)CH₃ | *1 |
| 1-111 | Me | O | CH₂CH₃ | CH₂CH₂S(O)₂CH₃ | *1 |
| 1-112 | Cl | O | CH₂CH₃ | CH(CH₃)S(O)CH₃ | *1 |
| 1-113 | Cl | O | CH₂CH₃ | CH(CH₃)S(O)₂CH₃ | *1 |
| 1-114 | Cl | O | CH₃ | CH₂S(O)CH₃ | 103-105 |
| 1-115 | Cl | O | CH₃ | CH₂S(O)₂CH₃ | 140-141 |
| 1-116 | Cl | O | CH₂CH₃ | CH(SCH₃)₂ | 93-95 |
| 1-117 | Cl | O | CH₂CH₃ | CH₂CH₂SH | *1 |
| 1-118 | Cl | O | CH₂CH₃ | CH(CH₃)SCH₂CH₃ | 65-66 |
| 1-118(+) | 99% e.e. | | [α]_{D}^{23.9} + 11.93° | (CHCl₃,c = 0.503) | 65-66 |
| 1-118(−) | 94.7% e.e. | | [α]_{D}^{23.8} − 11.18° | (CHCl₃,c = 0.340) | 65-66 |
| 1-119 | Me | O | CH₂CH₃ | N(CH₃)CH₂CH₂SCH₃ | *1 |
| 1-120 | Cl | O | CH₂CH₃ | CH(CH₂CH₃)SCH₃ | *2(~1:1) |
| 1-121 | Cl | O | CH₂CH₃ | CH(F)SCH₃ | *2(~3:2) |
| 1-122 | Cl | O | CH₂CH₃ | CH₂CH₂SCH₂CH₂CH₃ | *1 |
| 1-123 | Cl | O | CH₂CH₃ | CH₂CH₂SCH(CH₃)₂ | *1 |
| 1-124 | Cl | O | CH₂CH₃ | CH₂CH₂SCH₂CF₃ | *1 |
| 1-125 | Cl | O | CH₂CH₃ | CH{OC(O)CH₃}S(O)CH₃ | *2(~3:1) |
| 1-126 | Cl | O | CH₂CH₃ | CH(F)S(O)₂CH₃ | 133-135 |
| 1-127 | Cl | O | CH₂CH₃ | CH{OC(O)CH₃}S(O)₂CH₃ | *2(~3:2) |
| 1-128 | Cl | O | H | CH(Br)CH₂CH₂CH₂Br | 119-120 |
| 1-129 | Cl | O | —CH₂CH₂CH₂CH(Br)— | | 211-212 |
| 1-130 | Cl | O | —CH₂CH₂CH₂CH(SCH₃)— | | 118-119 |
| 1-131 | Cl | O | H | CH₂CH₂CH₂Cl | 97-98 |
| 1-132 | Cl | O | —CH₂CH₂CH₂— | | 109-110 |
| 1-133 | Cl | O | —CH₂CH₂C(═CH₂)— | | 117-118 |
| 1-134 | Cl | O | CH₃ | C(CH₃)₂CH₂SCH₃ | *1 |
| 1-135 | Cl | O | CH₃ | C(═CH₂)CH₂C(O)OH | 128-132 |
| 1-136 | Me | O | —C(O)CH(CH₂SCH₃)NH— | | 231-233 |
| 1-137 | Me | O | —C(O)CH{CH₂S(O)₂CH₃}NH— | | 200-205 |
| 1-138 | Me | O | CH₃ | NHCH₂CH₃ | 148-150 |
| 1-139 | Cl | O | CH₃ | C(CH₃)₂CH₂S(O)₂CH₃ | 130-135 |
| 1-140 | Cl | O | CH₂CH₃ | C(CH₃)₂SCH₃ | 116-117 |
| 1-141 | Cl | O | —CH₂CH₂CH(CH₂SCH₃)— | | 83-84 |
| 1-142 | Cl | O | CH₃ | C(═NOCH₃)CH₂SCH₃ | *1 |
| 1-143 | Cl | O | CH₂CH₃ | C(═NOCH₃)CH₂SCH₃ | *1 |
| 1-144 | Cl | O | CH₂C≡CH | CH₂CH₂SCH₃ | *1 |
| 1-145 | Cl | O | —CH₂CH₂CH{CH₂S(O)₂CH₃}— | | 198-199 |
| 1-146 | Cl | O | CH₂C≡CH | CH₂CH₂S(O)₂CH₃ | *1 |
| 1-147 | Cl | O | CH₂C≡CH | CH(CH₃)CH₂SCH₃ | *1 |
| 1-148 | Cl | O | CH₃ | CH₃ | 55-58 |
| 1-149 | Cl | O | CH₂CH₃ | CH₃ | *1 |
| 1-150 | Cl | O | —CH₂CH₂CH₂CH{S(O)₂CH₃}— | | 207-208 |
| 1-151 | Cl | O | CH₂CH₃ | CH₂CH₂CH₃ | *1 |
| 1-152 | Cl | O | CH₂CH₃ | CH═CH₂ | 67-68 |
| 1-153 | Cl | O | CH₂CH₂CH₂SCH₃ | CH₃ | *1 |
| 1-154 | Cl | O | CH₂CH₃ | CH₂CH₂S(O)CH₂CF₃ | 96-97 |
| 1-155 | Cl | O | CH₂CH₃ | CH₂CH₂S(O)₂CH₂CF₃ | *1 |
| 1-156 | Cl | O | CH₂CH₃ | CH(SCH₃)CH₂SCH₃ | *1 |
| 1-157 | Cl | O | CH₂CH₃ | C(═NOCH₂CH₃)CH₂SCH₃ | *1 |
| 1-158 | Cl | O | H | NHCH₂CF₃ | 228-230 |
| 1-159 | Cl | O | CH₂CH₃ | C(═CH₂)CH₂SCH₃ | *2(~3:2) |
| 1-160 | Cl | O | CH₂CH₃ | CH(OCH₃)CH₂SCH₃ | *2(~3:2) |
| 1-161 | Cl | O | CH₂CH₃ | C(═CH₂)CH₂SCH₂CH₃ | *1 |
| 1-162 | Cl | O | CH₂CH₃ | C(═NOCH₃)CH₂S(O)CH₃ | *2(~4:1) |
| 1-163 | Cl | O | CH₂CH₃ | C(═NOCH₃)CH₂S(O)₂CH₃ | 130-133 |
| 1-164 | Cl | O | H | CH₂SCH₃ | 183-184 |
| 1-165 | H | O | NH₂ | OC(CH₃)₃ | 120-123 |
| 1-166 | H | O | NHC(O)CH₂SCH₃ | OC(CH₃)₃ | *1 |
| 1-167 | Cl | O | CH₃ | CH(SCH₃)CH₂SCH₃ | *1 |
| 1-168 | H | S | —CH₂CH₂CH₂— | | 83-84 |
| 1-169 | Me | O | CH₃ | N(CH₂CH₃)C(O)CH₂SCH₃ | *1 |
| 1-170 | H | NOCH₃ | —CH₂CH₂CH₂— | | 75-76 |
| 1-171 | H | NOCH₃ | —CH₂CH₂CH₂— | | 55-56(*4) |
| 1-172 | Cl | O | CH₂CH₃ | C{═NOCH(CH₃)₂}CH₂SCH₃ | *1 |
| 1-173 | Cl | O | CH₂CH₃ | C(═NOCH₂CH₂CH₃)CH₂SCH₃ | *1 |
| 1-174 | Cl | O | CH₂CH₃ | CH₂C(O)OCH₃ | 102-103 |
| 1-175 | Cl | O | CH₃ | CH₂CN | 145-154 |
| 1-176 | Cl | O | CH₃ | CH(Cl)CH₂SCH₃ | 113-114 |
| 1-177 | Cl | O | CH₃ | CH₂CH₂SCH₂CF₃ | *1 |
| 1-178 | Cl | O | CH₃ | CH₂CH₂S(O)CH₂CF₃ | *1 |
| 1-179 | Cl | O | CH₃ | CH₂CH₂S(O)₂CH₂CF₃ | *1 |
| 1-180 | Me | O | —C(O)CH₂CH₂— | | 155-156 |
| 1-181 | Cl | O | CH₂CH₃ | CH{C(O)OCH₃}CH₂SCH₂CH₃ | *1 |
| 1-182 | Cl | O | CH₂CH₃ | CH(CH₂SCH₃)CH₂SCH₃ | *1 |
| 1-183 | Cl | O | CH₂CH₃ | C(═NOCH₂Ph)CH₂SCH₃ | *1 |
| 1-184 | Cl | O | CH₂CH₃ | C(═NOCH₂CH₂CH₂CH₃)CH₂SCH₃ | *1 |
| 1-185 | Cl | O | CH₃ | CH(CN)CH₂SCH₂CH₃ | *1 |
| 1-186 | Cl | O | CH₂CH₂SCH₃ | CH₃ | *1 |
| 1-187 | Cl | O | —CH₂C(OCH₃)₂— | | 135-138 |
| 1-188 | Br | O | H | OC(CH₃)₃ | 109-112 |
| 1-189 | Br | O | CH₂CH₃ | OC(CH₃)₃ | 84-86 |
| 1-190 | Cl | O | CH₃ | N(CH₃)CH₂CF₃ | *1 |
| 1-191 | Cl | O | CH₃ | C(═NOCH₃)CH₃ | *1 |
| 1-192 | Cl | O | CH₂CH₃ | CH(Cl)CH₂S(O)₂CH₃ | 162-163 |
| 1-193 | Me | O | CH₃ | C(═NOCH₃)CH₂SCH₃ | *1 |
| 1-194 | Me | O | CH₂CH₃ | C(═NOCH₃)CH₂SCH₃ | 71-73 |
| 1-195 | Cl | O | CH₂CH₃ | CH(CN)CH2SCH2CH3 | *1 |
| 1-196 | Cl | O | CH₃ | NHCH₂CF₃ | 199-201 |
| 1-197 | H | O | N(CH₃)C(O)CH(CH₃)SCH₃ | OC(CH₃)₃ | *2(~10:7) |
| 1-198 | Br | O | CH₂CH₃ | CH₂CH₂SCH₃ | *1 |
| 1-199 | Cl | O | CH₂CH₃ | CH(OCH₃)CH₂S(O)CH₃ | *2(~3:1) |
| 1-200 | Cl | O | CH₂CH₃ | CH(OCH₃)CH₂S(O)₂CH₃ | 174-176 |
| 1-201 | Cl | O | CH₂CH₃ | CH(CH₃)CH₂SCH₂CF₃ | *1 |
| 1-202 | Br | O | CH₂CH₃ | CH(CH₃)CH₂SCH₃ | *1 |
| 1-203 | Br | O | CH₂CH₃ | CH₂CH₂S(O)CH₃ | *1 |
| 1-204 | Br | O | CH₂CH₃ | CH₂CH₂S(O)₂CH₃ | *1 |
| 1-205 | Br | O | CH₂CH₃ | CH(CH₃)CH₂S(O)CH₃ | *2(~1:1) |
| 1-206 | Br | O | CH₂CH₃ | CH(CH₃)CH₂S(O)₂CH₃ | *1 |
| 1-207 | H | O | N(CH₃)C(O)CH₂SCH₃ | OC(CH₃)₃ | 91-92 |
| 1-208 | Cl | O | CH₂CH₃ | C(═NOCH₂CH₃)CH₂S(O)CH₃ | *1 |
| 1-209 | Cl | O | CH₂CH₃ | C(═NOCH₂CH₃)CH₂S(O)₂CH₃ | *1 |
| 1-210 | Cl | O | CH₂CH₃ | CH(CH₃)SCH₂CF₃ | *1 |
| 1-211 | Cl | O | CH₂CH₃ | C(═NOCH₃)CH₃ | *1 |
| 1-212 | Cl | O | CH₂CH₃ | Pr-c | 89-91 |
| 1-213 | Cl | O | CH₂CH₃ | D1-103b | *1 |
| 1-214 | Cl | O | CH₂CH₃ | D1-103c | *1 |
| 1-215 | Cl | O | CH₂CH₃ | D1-103d | *1 |
| 1-216 | Cl | O | CH₂CH₃ | CH(Ph)CH₂SCH₃ | *1 |
| 1-217 | Cl | O | CH₂CH₃ | CH(Ph)CH₂S(O)CH₃ | *2(~1:1) |
| 1-218 | Cl | O | CH₂CH₃ | CH(Ph)CH₂S(O)₂CH₃ | *1 |
| 1-219 | Cl | O | CH₂CH₃ | CH(CH₃)CH₂SC(O)CH₃ | *1 |
| 1-220 | Cl | O | CH₂CH₃ | CH₂OCH₂CH₃ | 93-96 |
| 1-221 | Cl | O | CH₂CH₃ | C(═NOCH₃)CH₂SCH₂CF₃ | *1 |
| 1-222 | Cl | O | CH₂CH₃ | C(═NOCH₂CH═CH₂)CH₂SCH₃ | *1 |
| 1-223 | Cl | O | CH₂CH₃ | C(═NOCH₂CH₂OCH₃)CH₂SCH₃ | *1 |
| 1-224 | Cl | O | CH₂CH₃ | CH(Br)CH₃ | 224-225 |
| 1-225 | Cl | O | CH₂CH₃ | CH(CH₃)S(O)CH₂CH₃ | *2(~3:1) |
| 1-226 | Cl | O | CH₂CH₃ | CH(CH₃)S(O)₂CH₂CH₃ | |
| 1-227 | Cl | O | CH₃ | C(═NOCH₃)CH₂S(O)₂CF₃ | *1 |
| 1-228 | Cl | O | CH₂CH₃ | C(═NOCH₃)CH₂S(O)₂CF₃ | *1 |
| 1-229 | Cl | O | CH₂CH₃ | CH(CH₃)CH₂S(O)CH₂CF₃ | *2(~3:1) |
| 1-230 | Cl | O | CH₂CH₃ | CH(CH₃)CH₂S(O)₂CH₂CF₃ | *1 |
| 1-231 | Cl | O | CH₃ | CH(CH₃)CH₂SCH₂CF₃ | *1 |
| 1-232 | Cl | O | CH₂CH₃ | CH₂SCH₂CF₃ | *1 |
| 1-233 | Cl | O | CH₂CH₃ | C(═NOCH₃)CH₂S(O)CH₂CF₃ | *1 |
| 1-234 | Cl | O | CH₂CH₃ | C(═NOCH₃)CH₂S(O)₂CH₂CF₃ | *1 |
| 1-235 | Cl | O | CH₃ | C(═NOCH₃)CH₂SCH₂CF₃ | *1 |
| 1-236 | Cl | O | CH₃ | C(═NOCH₃)CH₂S(O)CH₂CF₃ | *1 |
| 1-237 | Cl | O | CH₃ | C(═NOCH₃)CH₂S(O)₂CH₂CF₃ | *1 |
| 1-238 | H | O | —N═C(CH₂SCH₃)O— | | 130-131 |
| 1-239 | Cl | O | CH₃ | CH(CH₃)SCH₂CH₃ | 108-109 |
| 1-240 | Cl | O | CH₃ | CH(CH₃)SCH₂CF₃ | *1 |
| 1-241 | Cl | O | CH₃ | CH(CH₃)S(O)₂CH₂CF₃ | *1 |
| 1-242 | Cl | O | CH₃ | CH₂SCH₂CH₃ | *1 |
| 1-243 | Cl | O | CH₃ | CH₂S(O)CH₂CH₃ | *1 |
| 1-244 | Cl | O | CH₃ | CH₂S(O)₂CH₂CH₃ | 155-156 |
| 1-245 | Cl | O | CH₂CH₃ | CH(CH₃)SC(O)CH₃ | *1 |
| 1-246 | Cl | O | CH₂CH₃ | CH(CH₃)SCH₂CH₂CH₃ | *1 |
| 1-247 | Cl | O | CH₂CH₃ | CH(CH₃)S(O)₂CH(CH₃)₂ | 137-138 |
| 1-248 | Cl | O | CH₂CH₃ | CH(CH₃)SCH(CH₃)₂ | *1 |
| 1-249 | Cl | O | CH₂CH₃ | CH(CH₃)S(O)₂CH₂CH₂CH₃ | 114-115 |
| 1-250 | Cl | O | CH₂CH₃ | CH(CH₃)S(D1-37a) | 126-128 |
| 1-251 | Cl | O | CH₂CH₃ | CH(CH₃)SCN | *1 |
| 1-252 | Cl | O | CH₂CH₃ | CH(CH₃)SCH₂CN | *1 |
| 1-253 | Cl | O | CH₂CH₃ | CH(CH₃)S(O)CH₂CF₃ | *2(~54:46) |
| 1-254 | Cl | O | CH₂CH₃ | CH(CH₃)S(O)₂CH₂CF₃ | 119-121 |
| 1-255 | Cl | O | CH₃ | CH(CH₃)SCH₃ | 111-112 |
| 1-256 | H | O | N{C(O)OC(CH₃)₃}₂ | OC(CH₃)₃ | *1 |
| 1-257 | H | O | NHC(O)OC(CH₃)₃ | OC(CH₃)₃ | *1 |
| 1-258 | H | O | NHC(O)CH₂CH₂SCH₃ | OC(CH₃)₃ | 129-130 |
| 1-259 | Cl | O | CH₃ | C(═NOCH₃)CH₂SCH₂CH₃ | *1 |
| 1-260 | Cl | O | CH₃ | C(═NOCH₃)CH₂S(O)CH₂CH₃ | *2(~3:2) |
| 1-261 | Cl | O | CH₃ | C(═NOCH₃)CH₂S(O)₂CH₂CH₃ | 105-110 |
| 1-262 | Cl | O | CH₂CH₃ | C(═NOCH₃)CH₂SCH₂CH₃ | *1 |
| 1-263 | Cl | O | CH₂CH₃ | C(═NOCH₃)CH₂S(O)CH₂CH₃ | *2(~4:1) |
| 1-264 | Cl | O | CH₂CH₃ | C(═NOCH₃)CH₂S(O)₂CH₂CH₃ | 110-115 |
| 1-265 | Cl | O | CH₃ | CH(CH₃)S(O)CH₂CH₃ | 129-130 |
| 1-266 | Cl | O | CH₃ | CH(CH₃)S(O)₂CH₂CH₃ | 167-168 |
| 1-267 | Cl | O | CH₂CH₃ | C(═NOCH₂OCH₃)CH₂SCH₃ | *1 |
| 1-268 | Cl | O | CH₂CH₃ | C{═NOCH₂CH₂Si(CH₃)₃}CH₂SCH₃ | *1 |
| 1-269 | Cl | O | CH₂CH₃ | CH(CH₂CH₃)S(O)CH₃ | 105-106 |
| 1-270 | Cl | O | CH₃ | CH(CH₃)S(O)CH₃ | *2(~54:46) |
| 1-271 | Cl | O | CH₃ | CH(CH₃)S(O)₂CH₃ | 151-152 |
| 1-272 | Cl | O | CH₂CH₃ | CH(CH₃)OCH₂CF₃ | 99-101 |
| 1-273 | Cl | O | CH₃ | C(═NOCH₃)CH₂OCH₂CF₃ | *1 |
| 1-274 | Cl | O | CH₂CH₃ | C(═NOCH₃)CH₂OCH₂CF₃ | *1 |
| 1-275 | Cl | O | H | CH(CH₃)SCH₂CH₃ | 104-105 |
| 1-276 | Cl | O | CH₂OCH₃ | CH(CH₃)SCH₂CH₃ | 68-69 |
| 1-277 | Cl | O | CH₂CH₃ | CH(CH₃)SCH₂OCH₃ | *1 |
| 1-278 | Cl | O | CH₂CH₃ | CH(CH₃)SCH₂Pr-c | *1 |
| 1-279 | Cl | O | CH₂CH₃ | CH(CH₃)ON═CHCH₃ | *1 |
| 1-280 | Cl | O | CH₂CH₃ | CH(CH₂CH₃)S(O)₂CH₃ | 116-117 |
| 1-281 | Cl | O | CH₂CH₃ | CH(CH₂CH₃)SCH₂CH₃ | 67-68 |
| 1-282 | Cl | O | CH₂CH₃ | CH(CH₂CH₃)S(O)CH₂CH₃ | 109-110 |
| 1-283 | Cl | O | CH₂CH₃ | CH(CH₂CH₃)S(O)₂CH₂CH₃ | 89-90 |
| 1-284 | Cl | O | CH₂CN | CH(CH₃)SCH₂CH₃ | *1 |
| 1-285 | Cl | O | CH₂CH₃ | CH₂S(O)₂CH₂CF₃ | *1 |
| 1-286 | Cl | O | CH₂CH₃ | CH(CH₃)SCH₂SCH₃ | *1 |
| 1-287 | Cl | O | CH₂CH₃ | CH(CH₃)SCH₂C≡CH | 82-83 |
| 1-288 | Cl | O | CH₃ | CH₂SCH₂CF₃ | *1 |
| 1-289 | Cl | O | CH₂C≡CH | CH(CH₃)SCH₂CH₃ | *1 |
| 1-290 | Br | O | CH₂CH₃ | CH(CH₃)SCH₃ | 93-95 |
| 1-291 | Br | O | CH₂CH₃ | CH(CH₃)SCH₂CH₃ | *1 |
| 1-292 | Cl | O | CH₂CH₃ | CH(CH₃)SCH₂C(O)NHCH₃ | *1 |
| 1-293 | Cl | O | CH₂CH₃ | CH(CH₃)SCH₂C(O)OCH₃ | *1 |
| 1-294 | Cl | O | CH₃ | CH(CH₂CH₃)SCH₃ | 86-87 |
| 1-295 | Cl | O | CH₃ | CH(CH₂CH₃)SCH₂CH₃ | 76-77 |
| 1-296 | Cl | O | CH₂CH₃ | CH₂S(O)CH₂CF₃ | 114-116 |
| 1-297 | Cl | O | CH₃ | CH(CH₂CH₃)S(O)CH₃ | 84-86 |
| 1-298 | Cl | O | CH₃ | CH(CH₂CH₃)S(O)₂CH₃ | 168-170 |
| 1-299 | Cl | O | CH₃ | CH(CH₂CH₃)S(O)₂CH₂CH₃ | 93-95 |
| 1-300 | Cl | O | CH₂C(O)OCH₂CH₃ | CH(CH₃)SCH₂CH₃ | *1 |
| 1-301 | Cl | O | S(O)₂CH₃ | CH(CH₃)SCH₂CH₃ | 125-127 |
| 1-302 | Cl | O | C(O)OCH₃ | CH(CH₃)SCH₂CH₃ | *1 |
| 1-303 | Cl | O | CH₂CH₃ | CH₂C(═NOMe)CF₃ | *1 |
| 1-304 | Cl | O | CH₃ | CH₂S(O)₂CH₂CF₃ | *1 |
| 1-305 | Cl | O | CH₂CH₃ | CH(CH₃)S(═NCN)CH₂CH₃ | *1 |
| 1-306 | Cl | O | CH₂CH₃ | D1-82a | 107-109 |
| 1-307 | Cl | O | CH₂CH₃ | D1-103e | *1 |
| 1-308 | Cl | O | CH₃ | CH₂NHC(O)OC(CH₃)₃ | *1 |
| 1-309 | Cl | O | CH₂OCH₃ | CH(CH₃)S(O)CH₂CH₃ | *1 |
| 1-310 | Cl | O | CH₂OCH₃ | CH(CH₃)S(O)₂CH₂CH₃ | *1 |
| 1-311 | Cl | O | CH₃ | CH₂S(O)CH₂CF₃ | 55-58 |
| 1-312 | Cl | O | CH₃ | CH₂NH₂ HCl | 168-180 |
| 1-313 | Cl | O | CH₃ | CH₂NHS(O)₂CH₃ | *1 |
| 1-314 | Cl | O | CH₃ | CH(CH₂CH₃)S(O)CH₂CH₃ | *2(~52:48) |
| 1-315 | Cl | O | CH₂CH₃ | CH(CH₃)SC(CH₃)₃ | *1 |
| 1-316 | Cl | O | CH₂CH═CH₂ | CH(CH₃)SCH₂CH₃ | 70-74 |
| 1-317 | Cl | O | CH₂CH₂CH₃ | OC(CH₃)₃ | 85-87 |
| 1-318 | Cl | S | CH₂CH₃ | CH(CH₃)SCH₂CH₃ | *1 |
| 1-319 | Cl | O | SC(Cl)₃ | CH(CH₃)SCH₂CH₃ | *1 |
| 1-320 | Cl | O | CH₂CH₃ | CH(CH₃)SCH₂(D1-34a) | *1 |
| 1-321 | Cl | O | CH₂CH₃ | CH(CH₃)SCH₂Si(CH₃)₃ | 98-100 |
| 1-322 | Cl | O | CH₂CH₃ | D1-82c | 192-195 |
| 1-323 | Cl | O | CH₂CH₂CH₃ | CH(CH₃)SCH₂CH₃ | 73-76 |
| 1-324 | Cl | O | CH₂CF₃ | OC(CH₃)₃ | *1 |
| 1-325 | Cl | O | CH₃ | CH₂N(CH₃)S(O)₂CH₃ | 156-158 |
| 1-326 | Cl | O | CH₃ | D1-10e(2-CF₃) | 151-153 |
| 1-327 | Cl | O | CH₃ | D1-19a | 117-119 |
| 1-328 | Cl | O | CH₃ | D1-32b(2-Br) | *1 |
| 1-329 | Cl | O | CH₃ | CH₂(D1-2a) | 96-98 |
| 1-330 | Cl | O | CH₃ | CH₂{D1-8f[X^{1a} = CH₃, X¹ = 3,5-(CH₃)₂} | 150-152 |
| 1-331 | Cl | O | CH₃ | CH₂{D1-33b(6-Cl)} | *1 |
| 1-332 | Cl | O | H | CH₂(D1-32a) | 154-155 |
| 1-333 | Cl | O | CH₃ | D1-1b(5-Br) | *1 |
| 1-334 | Cl | O | CH₃ | D1-33b(2-OCH₃) | 164-166 |
| 1-335 | Cl | O | CH₃ | D1-10f{2-(CH₂SCH₃),4-CH₃} | *1 |
| 1-336 | Cl | O | CH₃ | D1-38b(4-SCH₃) | *1 |
| 1-337 | Cl | O | CH₃ | CH₂(D1-33a) | *1 |
| 1-338 | Cl | O | CH₃ | CH₂(D1-34a) | 121-125 |
| 1-339 | Cl | O | CH₃ | CH₂(D1-28a) | 190-198 |
| 1-340 | Cl | O | CH₂CH₃ | CH(CH₃)SCH₂CH₂CH₂Cl | *1 |
| 1-341 | Cl | O | CH₂CF₃ | CH(CH₃)CH₂SCH₃ | *1 |
| 1-342 | Cl | O | CH₂CF₃ | CH(CH₃)SCH₂CH₃ | *1 |
| 1-343 | Cl | O | CH₂Pr-c | OC(CH₃)₃ | *1 |
| 1-344 | Cl | O | CH₃ | D1-2c(5-SCH₃) | *1 |
| 1-345 | Cl | O | CH₃ | D1-12f(X^{1a} = CH₃, X¹ = 4-NO₂) | 125-131 |
| 1-346 | Cl | O | CH₃ | D1-35b | *1 |
| 1-347 | Cl | O | CH₃ | CH₂{D1-5f(3-CH₃)} | 157-159 |
| 1-348 | Cl | O | CH₃ | D1-5e{3,5-(CH₃)₂} | *1 |
| 1-349 | Cl | O | CH₂CH₃ | D1-13d(CH₃) | *1 |
| 1-350 | Cl | O | CH₂CH₃ | CH₂{D1-8f[X^{1a} = CH₃, X¹ = 3,5-(Cl)₂]} | *1 |
| 1-351 | Cl | O | CH₂CH₃ | CH₂CH₂{D1-7b(3-CF₃,5-Pr-c)} | *1 |
| 1-352 | Cl | O | CH₂CH₃ | CH₂P(═O)(OCH₂CH₃)₂ | *1 |
| 1-353 | Cl | S | H | CH(CH₃)SCH₂CH₃ | *1 |
| 1-354 | Cl | O | CH₂CH₃ | CH(CH₃)S(D1-51a) | *1 |
| 1-355 | Cl | O | CH₂CH₃ | D1-45d{5,5-(CH₃)₂} | *1 |
| 1-356 | Cl | NOCH₃ | H | CH(CH₃)SCH₂CH₃ | *2(~5:2) |
| 1-357 | Cl | O | CH₂CH₃ | C≡CSi(CH₃)₃ | 94-95 |
| 1-358 | Cl | O | CH₂CH₃ | D1-88a{X^{1a} = C(O)OC(CH₃)₃} | 54-55 |
| 1-359 | Cl | O | CH₂CH₃ | C≡CH | *1 |
| 1-360 | Me | O | CH₂CH₃ | CH(CH₃)SCH₂CH₃ | *1 |
| 1-361 | Cl | O | CH₂CH₃ | D1-6d(4,5-Cl₂) | *1 |
| 1-362 | Cl | O | CH₂CH₃ | D1-2c{5-C(O)CH₃} | *1 |
| 1-363 | Cl | O | CH₂CH₃ | CH₂CH₂{D1-7b(3-CF₃,5-CH₃)} | *1 |
| 1-364 | H | O | CH₃ | Pr-c | 62-66 |
| 1-365 | Cl | O | CH₂CH₃ | CH₂NHC(O)OC(CH₃)₃ | 67-69 |
| 1-366 | Cl | O | CH₂CH₃ | D1-88a{X^{1a} = C(O)CH₃} | 140-141 |
| 1-367 | Cl | O | CH₂CH₃ | CH₂C(═NOCH₃)CH₂SCH₃ | *2(~65:35) |
| 1-368 | Cl | O | CH₂CH₃ | D1-87a | 149-150 |
| 1-369 | Cl | O | CH2Pr-c | CH(CH₃)SCH₂CH₃ | 75-80 |
| 1-370 | Me | O | CH₂CH₃ | CH(CH₃)S(O)₂CH₂CH₃ | *1 |
| 1-371 | Cl | O | CH₂Pr-c | CH(CH₃)CH₂SCH₃ | *1 |
| 1-372 | Cl | O | CH₂CH₃ | CH₂C(O)NHCH₃ | 91-93 |
| 1-373 | Cl | O | CH₂CH₃ | D1-2c{5-C(═NOCH₃)CH₃} | *2(~4:3) |
| 1-374 | OCH₃ | O | H | OC(CH₃)₃ | 141-143 |
| 1-375 | Cl | O | CH₂CH₃ | CH₂NHC(O)NHCH₂CH₃ | 160-162 |
| 1-376 | Cl | O | CH₂CH₃ | CH₂NHC(O)CH₂OCH₃ | *1 |
| 1-377 | Cl | O | CH₂CH₃ | D1-33b(6-CN) | *1 |
| 1-378 | Cl | O | CH₂CH₃ | C(O)(D1-2a) | *1 |
| 1-379 | Cl | O | CH₂CH₃ | D1-81a | 81-83 |
| 1-380 | Cl | O | CH₂CH₃ | D1-81b | *1 |
| 1-381 | Cl | O | CH₂CH₃ | CH₂CH₂CH₂CH₂Br | 109-110 |
| 1-382 | Cl | O | —CH₂CH₂CH₂CH₂— | | 125-126 |
| 1-383 | Cl | O | CH₂OCH₂CH₃ | CH(CH₃)SCH₂CH₃ | *1 |
| 1-384 | Cl | O | CH₂OCH₂CH₃ | CH(CH₃)S(O)₂CH₂CH₃ | 128-129 |
| 1-385 | Cl | O | CH₂CH₂OCH₃ | OC(CH₃)₃ | 75-76 |
| 1-386 | Cl | O | CH₂CH₂OCH₃ | CH(CH₃)SCH₂CH₃ | *1 |
| 1-387 | Cl | O | CH₂CH₂OCH₃ | CH(CH₃)S(O)₂CH₂CH₃ | *1 |
| 1-388 | Cl | O | CH₂CH₃ | CH(CH₃)SCH₂Ph | *1 |
| 1-389 | Cl | O | CH₂CH₃ | CH₂(D1-87a) | 117-118 |
| 1-390 | Cl | O | CH₂CH₃ | CH₂(D1-93a) | *1 |
| 1-391 | Cl | O | CH₂CH₃ | CH₂NHC(O)CH₂SCH₃ | 125-126 |
| 1-392 | Cl | O | CH₂CH₃ | CH₂NH₂ | 140-143 |
| 1-393 | Cl | O | CH₂CH₃ | D1-88a{X^{1a} = C(O)CF₃} | 135-137 |
| 1-394 | Cl | O | CH₂CH₃ | CH₂NHC(O)CH₃ | 128-129 |
| 1-395 | OCH₃ | O | CH₂CH₃ | OC(CH₃)₃ | *1 |
| 1-396 | Cl | O | CH₂Si(CH₃)₃ | OC(CH₃)₃ | 80-83 |
| 1-397 | Cl | O | CH₂CH₃ | CH(CH₃)S{D1-32b(3-CF₃)} | *1 |
| 1-398 | Cl | O | CH₂C(═NOCH₃)CH₃ | OC(CH₃)₃ | 97-98 |
| 1-399 | Cl | O | CH₂C(═NOCH₃)CH₃ | CH(CH₃)CH₂SCH₃ | *2(~9:1) |
| 1-400 | Cl | O | CH₂Si(CH₃)₃ | CH(CH₃)CH₂SCH₃ | *1 |
| 1-401 | Cl | O | CH₂Si(CH₃)₃ | CH(CH₃)SCH₂CH₃ | 66-70 |
| 1-402 | Cl | O | CH₂CH₃ | D1-94c{X^{1a} = C(O)OC(CH₃)₃} | *1 |
| 1-403 | Cl | O | CH₂CH₃ | D1-94b{X^{1a} = C(O)OC(CH₃)₃} | *1 |
| 1-404 | Cl | O | CH₂CH₃ | D1-10f{2-CH₂OCH₃, 4-CF₃} | *1 |
| 1-405 | Cl | O | CH₂CH₃ | D1-8f{X^{1a} = CH₃, X¹ = 3-Cl, 5-S(O)₂NH₂} | *1 |
| 1-406 | Cl | O | CH₂CH₃ | D1-4a(X^{1a} = CH₃) | *1 |
| 1-407 | Cl | O | CH₂CH₃ | CH₂NHS(O)₂N(CH₃)₂ | *1 |
| 1-408 | Cl | O | CH₂CH₃ | CH₂NHC(S)NHCH₂CH₃ | 162-164 |
| 1-409 | Cl | O | CH₂CH₃ | CH₂NHC(O)CF₃ | *1 |
| 1-410 | Cl | O | CH₂CH₃ | CH₂NHS(O)₂CF₃ | 140-145 |
| 1-411 | Cl | O | CH₂CH₃ | CH₂N(CH₃)₂ | *1 |
| 1-412 | Cl | O | —CH₂CH₂CH═CH— | | 109-110 |
| 1-413 | Cl | O | H | NHCH═NOCH₃ | >280 |
| 1-414 | Cl | O | H | N═CHN(CH₃)₂ | 105-110 |
| 1-415 | Cl | O | H | NH₂ | >280 |
| 1-416 | Cl | O | CH₂CH₃ | CH(CH₃)NHC(O)OC(CH₃)₃ | 193-195 |
| 1-417 | Cl | O | CH₂CH₃ | CH₂N(CH₃)C(O)OC(CH₃)₃ | *1 |
| 1-418 | Cl | O | CH₂CH₃ | CH₂CH₂C(F)═CF₂ | *1 |
| 1-419 | Cl | O | CH₂CH₃ | CH2{D1-108b(2,3-OCH₂O—)} | 85-89 |
| 1-420 | Cl | O | CH₂CH₃ | CH₂CH₂NHC(O)OC(CH₃)₃ | *1 |
| 1-421 | Cl | O | CH₂CH₃ | CH═CH(D1-33a) | *1 |
| 1-422 | Cl | O | CH₂CH₃ | CH═CH(D1-1a) | *1 |
| 1-423 | Cl | O | CH₂CH₃ | CH═CH(D1-2a) | *1 |
| 1-424 | Cl | O | CH₂CH₃ | C(O)OCH₂CH₃ | *1 |
| 1-425 | Cl | O | CH₂CH₃ | Pen-c | 76-78 |
| 1-426 | Cl | O | CH₂CH₃ | C(CH₃)₂OC(O)CH₃ | *1 |
| 1-427 | Cl | O | CH₂CH₃ | CF₂CF₂Cl | *1 |
| 1-428 | CF₃ | O | H | OC(CH₃)₃ | 86-88 |
| 1-429 | CF₃ | O | CH₂CH₃ | OC(CH₃)₃ | *1 |
| 1-430 | Cl | O | CH₂SCH₃ | CH(CH₃)SCH₂CH₃ | *1 |
| 1-431 | Cl | O | CH₂CH₃ | CH(CH₃)S{D1-12a(X^{1a} = CH₃)} | *1 |
| 1-432 | Cl | O | CH₂CH₃ | CH(CH₃)S(D1-32a) | *1 |
| 1-433 | Cl | O | CH₂CH₃ | CH(CH₃)S{D1-32b(3-NO₂)} | *1 |
| 1-434 | Cl | O | CH₂CH₃ | CH₂SCH₂CH(OCH₃)₂ | *1 |
| 1-435 | CH₂CH₃ | O | H | OC(CH₃)₃ | *1 |
| 1-436 | C(O)OCH₃ | O | H | CH(CH₃)SCH₂CH₃ | 102-104 |
| 1-437 | CH₂OH | O | H | CH(CH₃)SCH₂CH₃ | 87-96 |
| 1-438 | Cl | O | CH₂CH₃ | D1-88a{X^{1a} = C(O)CH₂SCH₃} | 45-47 |
| 1-439 | Cl | O | CH₂CH₃ | CH₂OC(CH₃)₃ | *1 |
| 1-440 | Cl | O | CH₂CH₃ | D1-92c | *1 |
| 1-441 | Cl | O | CH₂CH₃ | CH(CH₃)OCH₂CH═CH₂ | 91-93 |
| 1-442 | Cl | O | CH₂CH₃ | CH(CH₃)OCH₂C≡CH | 105-107 |
| 1-443 | Cl | O | CH₂CH₃ | D1-103h | *1 |
| 1-444 | Cl | O | CH₂CH₃ | CH₂C(CH₃)₃ | *1 |
| 1-445 | Cl | O | CH₂CH₃ | C(CH₃)═CH₂ | *1 |
| 1-446 | Cl | O | CH₂CH₃ | CHCl₂ | 81-83 |
| 1-447 | Cl | O | CH₂CH₃ | D1-103i | 68-70 |
| 1-448 | Cl | O | CH₂CH₃ | CH(Br)C(CH₃)₃ | 99-101 |
| 1-449 | Cl | O | CH₂CH₃ | NHPh | 163-165 |
| 1-450 | Cl | S | H | NHCH₂CH═CH₂ | 168-170 |
| 1-451 | Cl | S | H | NHPr-c | 192-195 |
| 1-452 | Cl | O | CH₂CH₃ | CH₂(D1-98a) | *1 |
| 1-453 | CH₂OCH₃ | O | CH₃ | CH(CH₃)SCH₂CH₃ | *1 |
| 1-454 | Cl | O | C(O)CH₃ | CH₃ | 106-107 |
| 1-455 | Cl | O | CH₂CH₃ | C(═NOCH₃)C(═NOCH₃)CH₃ | 130-131 |
| 1-456 | Cl | O | CH₂CH₃ | CH₂CH₂CH₂CH₂CH₂CH₂CH₂CH₃ | *1 |
| 1-457 | Cl | O | CH₂CH₃ | CH₂CH₂CH₂CH₂CH₂CH₂CH₂CH₂CH₂CH₂CH₃ | *1 |
| 1-458 | Cl | O | CH₂CH₃ | D1-103f | *1 |
| 1-459 | Cl | O | CH₂CH₃ | Bu-c | *1 |
| 1-460 | Cl | O | CH₂CH₃ | CH₂CH(CF₃)₂ | 74-76 |
| 1-461 | Cl | O | CH₂CH₃ | CH₂Pr-c | *1 |
| 1-462 | Cl | O | CH₂CH₃ | CH₂{D1-85d[X^{1b} = C(Cl)₃] | *1 |
| 1-463 | Cl | O | CH₂CH₃ | CH2{D1-84d[X^{1b} = C(Cl)₃] | *1 |
| 1-464 | Cl | O | CH₂CH₃ | C(O)NHPh | 156-158 |
| 1-465 | Cl | O | CH₂CH₃ | CH₂SCH₂CH(═NOCH₃) | *2(~2:1) |
| 1-466 | Cl | O | CH₂CH₃ | NH(D1-33a) | 184-186 |
| 1-467 | SCH₃ | O | H | OC(CH₃)₃ | *1 |
| 1-468 | Cl | O | CH₂CH₃ | D1-103g | 57-60 |
| 1-469 | Cl | O | CH₃ | D1-108b(2-Cl) | *1 |
| 1-470 | Cl | O | CH₃ | D1-108b(3-Cl) | 121-131 |
| 1-471 | Cl | O | CH₃ | D1-108b(4-Cl) | 160-161 |
| 1-472 | Cl | O | CH₂CH₃ | D1-108b(2-SCH₃) | *1 |
| 1-473 | Cl | O | CH₂CH₃ | D1-108b(3-SCH₃) | *1 |
| 1-474 | Cl | O | CH₂CH₃ | D1-108b(4-SCH₃) | *1 |
| 1-475 | Cl | O | CH₂CH₃ | D1-108b(3-SCF₃) | *1 |
| 1-476 | Cl | O | CH₂CH₃ | D1-108b(3,4-OCF₂O—) | *1 |
| 1-477 | Cl | O | CH═NOCH₃ | CH(CH₃)CH₂SCH₃ | *2(~4:1) |
| 1-478 | Cl | S | H | NHCH₂CH₃ | 107-108 |
| 1-479 | H | O | H | CH(CH₃)SCH₂CH₃ | 92-93 |
| 1-480 | H | S | H | CH(CH₃)SCH₂CH₃ | 143-144 |
| 1-481 | H | NOCH₃ | H | CH(CH₃)SCH₂CH₃ | *2(~11:9) |
| 1-482 | CH₂Cl | O | H | CH(CH₃)SCH₂CH₃ | 88-90 |
| 1-483 | Cl | O | CH₂CH₃ | CH(CH₃)CH₂CH₂CH₃ | 60-61 |
| 1-484 | CH₂CH₃ | O | CH₂CH₃ | CH(CH₃)CH₂SCH₃ | *1 |
| 1-485 | Cl | S | CH₂CH₃ | NHPr-c | 142-144 |
| 1-486 | H | NOCH₃ | CH₃ | CH(CH₃)SCH₂CH₃ | *2(~7:1) |
| 1-487 | S(O)₂CH₃ | O | CH₂CH₃ | OC(CH₃)₃ | *1 |
| 1-488 | Cl | O | CH₂CH₃ | CH(CH₃)CH₂S(═NCN)CH₃ | *1 |
| 1-489 | CH₃ | O | CH₂CH₃ | CH(CH₃)CH₂S(O)CH₃ | *1 |
| 1-490 | CH₃ | O | CH₂CH₃ | CH(CH₃)CH₂S(O)₂CH₃ | *1 |
| 1-491 | Cl | O | CH₂CH₃ | CH(CH₃)CH₂S(O)(═NCN)CH₃ | *1 |
| 1-492 | S(O)₂CH₃ | O | H | OC(CH₃)₃ | 155-157 |
| 1-493 | Cl | O | H | CH(CH₃)CH₂S(O)CH₃ | 171-172 |
| 1-494 | Cl | O | H | CH(CH₃)CH₂S(O)₂CH₃ | 180-181 |
| 1-495 | Cl | O | SC(Cl)₃ | CH(CH₃)CH₂SCH₃ | *1 |
| 1-496 | Cl | O | CH═NOCH₃ | CH₂CH₂SCH₃ | *1 |
| 1-497 | Cl | NCH₂CH₃ | CH₃ | SCH₃ | *1 |
| 1-498 | Cl | O | CH₂CH₃ | D1-9f(4-CH₃) | *1 |
| 1-499 | Cl | O | CH₂CH₃ | NHPr-c | 180-182 |
| 1-500 | Cl | O | CH₂CH₃ | N(CH₃)Pr-c | *1 |
| 1-501 | Cl | O | CH₂CH₃ | NHCH₂CF₃ | 152-153 |
| 1-502 | CN | O | CH₂CH₃ | OC(CH₃)₃ | *1 |
| 1-503 | Br | O | CH₃ | OC(CH₃)₃ | *1 |
| 1-504 | Cl | O | CH₂CH₃ | N(CH₃)CH₂CF₃ | *1 |
| 1-505 | Cl | O | CH₂Pr-c | CH₂CH₂SCH₃ | *1 |
| 1-506 | Cl | O | CH₂Pr-c | CH(CH₃)CH₂S(O)CH₃ | *2(~10:4) |
| 1-507 | Cl | O | CH₂Pr-c | CH₂CH₂S(O)CH₃ | *2(~10:3) |
| 1-508 | Cl | O | CH₂Pr-c | CH(CH₃)S(O)CH₂CH₃ | *2(~10:1) |
| 1-509 | Cl | O | CH₂Pr-c | CH(CH₃)CH₂S(O)₂CH₃ | *1 |
| 1-510 | Cl | O | CH₂Pr-c | CH₂CH₂S(O)₂CH₃ | *1 |
| 1-511 | Cl | O | CH₂Pr-c | CH(CH₃)S(O)₂CH₂CH₃ | 123-125 |
| 1-512 | Cl | O | CH₂CH₃ | NH(D1-80a) | 236-237 |
| 1-513 | Cl | O | CH₃ | CH(CH₃)CH₂SCH₂CH₃ | 50-54 |
| 1-514 | Cl | O | CH₂CH₃ | CH(CH₃)CH₂SCH₂CH₃ | *1 |
| 1-515 | Cl | O | CH₃ | CH(CH₃)CH₂S(O)CH₂CH₃ | *2(~2:1) |
| 1-516 | Cl | O | CH₂CH₃ | CH(CH₃)CH₂S(O)CH₂CH₃ | *2(~2:1) |
| 1-517 | Cl | O | CH₃ | CH(CH₃)CH₂S(O)₂CH₂CH₃ | *1 |
| 1-518 | Cl | O | CH₂CH₃ | CH(CH₃)CH₂S(O)₂CH₂CH₃ | *1 |
| 1-519 | Cl | O | CH₂CH₃ | NHC(O)CF₃ | 175-179 |
| 1-520 | Cl | O | CH₂CH₃ | CH₂CH₂CH₂SCH₃ | *1 |
| 1-521 | Cl | O | CH₂CH₃ | CH₂C(O)NHCH₂CF₃ | 115-125 |
| 1-522 | Cl | O | CH₂CN | OC(CH₃)₃ | 77-79 |
| 1-523 | CF₃ | O | CH₂CH₃ | CH(CH₃)CH₂SCH₃ | *1 |
| 1-524 | S(O)₂CH₃ | O | CH₂CH₃ | CH(CH₃)CH₂SCH₃ | *1 |
| 1-525 | CN | O | CH₂CH₃ | CH(CH₃)CH₂SCH₃ | *1 |
| 1-526 | OCH₃ | O | CH₂CH₃ | CH(CH₃)CH₂SCH₃ | *1 |
| 1-527 | Cl | O | CH₂CH₃ | CH₂CH₂CH₂S(O)CH₃ | *1 |
| 1-528 | Cl | O | CH₂CH₃ | CH₂CH₂CH₂S(O)₂CH₃ | *1 |
| 1-529 | Cl | O | CH₂CH₃ | CH₂C(O)N(CH₃)S(O)₂CH₃ | 65-72 |
| 1-530 | Cl | O | CH₂CH₃ | N(CH₂CH₃)CH₂CF₃ | *1 |
| 1-531 | Cl | O | CH₂CH₃ | CH(CH₃)S{D1-108b(4-F)} | *1 |
| 1-532 | Cl | O | CH₂CH₃ | CH(CH₃)S{D1-108b(3-F)} | *1 |
| 1-533 | Cl | O | CH₂CH₃ | CH(CH₃)S{D1-108b(2-F)} | *1 |
| 1-534 | Cl | O | CH₂CH₃ | CH(CH₃)S{D1-108b(4-CH₃)} | *1 |
| 1-535 | Cl | O | CH₂CH₃ | CH(CH₃)S{D1-108b(4-CF₃)} | *1 |
| 1-536 | Cl | O | CH₂CH₃ | N(CH₂SCH₃)CH₂CF₃ | *1 |
| 1-537 | Cl | O | CH₂CN | CH(CH₃)S(O)CH₂CH₃ | 44-47 |
| 1-538 | Cl | O | CH₂CN | CH(CH₃)S(O)₂CH₂CH₃ | 136-138 |
| 1-539 | Cl | O | CH₂CH₃ | CH═CHCH₂SCH₃ | *2(~78:22) |
| 1-540 | Cl | O | CH₂CH₃ | N{CH₂S(O)₂CH₃}CH₂CF₃ | 162-163 |
| 1-541 | Cl | S | CH₂CH₃ | N(CH₃)CH₂CF₃ | *1 |
| 1-542 | Cl | O | CH₂CN | CH₂CH₂SCH₃ | *1 |
| 1-543 | Cl | O | CH₂CN | CH₂CH₂S(O)CH₃ | *1 |
| 1-544 | Cl | O | CH₂CN | CH₂CH₂S(O)₂CH₃ | *1 |
| 1-545 | Br | O | CH₃ | CH(CH₃)CH₂SCH₃ | *1 |
| 1-546 | Br | O | CH₃ | C(═NOCH₃)CH₂SCH₃ | *1 |
| 1-547 | Cl | O | CH₂CN | CH(CH₃)CH₂SCH₃ | 126-127 |
| 1-548 | Me | O | H | NHCH₂CH₃ | 199-200 |
| 1-549 | Me | S | H | NHCH₂CH₃ | 120-122 |
| 1-550 | Cl | O | H | NHCH₂CH₃ | 215-217 |
| 1-551 | Cl | O | CH₃ | NHCH₂CH₃ | 167-168 |
| 1-552 | Cl | S | H | NHCH₃ | 180-183 |
| 1-553 | Cl | S | H | NHCH₂CH₂CH₃ | 167-169 |
| 1-554 | Cl | S | H | NHCH(CH₃)₂ | 176-178 |
| 1-555 | Cl | S | H | NH{D1-108b(4-SCH₃)} | 148-150 |
| 1-556 | Cl | S | H | NHPh | 161-163 |
| 1-557 | Cl | S | H | NH{D1-108b(4-CF₃)} | 216-218 |
| 1-558 | Cl | S | H | NH{D1-108b(3-CF₃)} | 153-155 |
| 1-559 | Cl | S | H | NH{D1-108b(2-CF₃)} | 130-132 |
| 1-560 | Cl | S | H | NHCH₂(D1-108a) | 147-149 |
| 1-561 | Cl | S | H | NHC(O)OCH₂CH₃ | 240-242 |
| 1-562 | Cl | S | CH₃ | NHCH₂CH₃ | 141-142 |
| 1-563 | Cl | O | CH₂CH₃ | NHCH₂CH₃ | 169-171 |
| 1-564 | Cl | S | CH₂CH₃ | NHCH₂CH₃ | 127-128 |
| 1-565 | Me | O | CH₂CH₃ | NHCH₂CH₃ | 115-120 |
| 1-566 | Me | S | CH₂CH₃ | NHCH₂CH₃ | 129-131 |
| 1-567 | Me | S | CH₃ | NHCH₂CH₃ | 167-169 |
| 1-568 | Cl | O | CH₂CN | CH(CH₃)CH₂S(O)CH₃ | *2(~3:2) |
| 1-569 | Cl | O | CH₂CN | CH(CH₃)CH₂S(O) ₂CH₃ | *1 |
| 1-570 | Cl | O | CH₂CH₃ | NHCH₃ | 168-170 |
| 1-571 | Me | O | CH₂CH₃ | NHCH₂CF₃ | 112-113 |
| 1-572 | Cl | O | CH₂CH₃ | NH₂ | 204-206 |
| 1-573 | Cl | O | CH₂CH₃ | N(CH₂OCH₃)CH₂CF₃ | *1 |
| 1-574 | Cl | O | CH₂CH₃ | NCH₂CH₂Cl | 130-132 |
| 1-575 | Cl | O | CH₂CH₃ | NCH₂CH₂CH₃ | 124-128 |
| 1-576 | Cl | O | CH₂CH₃ | NCH(CH₃)₂ | 130-134 |
| 1-577 | Cl | S | H | NHCH₂C≡CH | 210-212 |
| 1-578 | Cl | S | H | NH(D1-33a) | 100-102 |
| 1-579 | Cl | S | H | NHCH₂CF₃ | 177-178 |
| 1-580 | Cl | S | H | N(CH₃)CH₂CH₃ | 134-135 |
| 1-581 | Br | O | CH₃ | CH(CH₃)CH₂S(O)CH₃ | *2(~7:5) |
| 1-582 | Br | O | CH₃ | CH(CH₃)CH₂S(O)₂CH₃ | *1 |
| 1-583 | Br | O | CH₃ | C(═NOCH₃)CH₂S(O)CH₃ | *1 |
| 1-584 | Br | O | CH₃ | C(═NOCH₃)CH₂S(O)₂CH₃ | *1 |
| 1-585 | Br | O | H | NHCH₂CF₃ | >280 |
| 1-586 | Br | O | CH₃ | NHCH₂CF₃ | 209-210 |
| 1-587 | Br | O | CH₂CH₃ | NHCH₂CF₃ | 145-147 |
| 1-588 | Cl | O | CH₂CH₃ | CH(CH₃)(D1-87a) | *1 |
| 1-589 | Cl | O | CH₂CH₃ | D1-108b{2-C(O)OH} | 177-179 |
| 1-590 | Cl | O | CH₂CH₃ | D1-108b{2-C(O)NHS(O)₂CH₃) | *1 |
| 1-591 | Cl | O | —CH₂CH₂N(CH₂CF₃)— | | 130-132 |
| 1-592 | Cl | O | —CH₂CH₂NH— | | 170-172 |
| 1-593 | Cl | O | H | NHCH₂CH₂Cl | 182-184 |
| 1-594 | Cl | S | CH₂CH₃ | NH(D1-34a) | 96-98 |
| 1-595 | Cl | O | CH₂CH₃ | NHCH₂CH═CH₂ | 145-147 |
| 1-596 | Cl | O | CH₂CH₃ | CH(CH₃)CH₂S(O)₂N(CH₃)₂ | *1 |
| 1-597 | Cl | S | CH₂CH₃ | NH(D1-32a) | 241-242 |
| 1-598 | Cl | O | CH₂CH₃ | NHCH₂CN | *1 |
| 1-599 | Cl | O | CH₂CH₃ | NHCH₂(D1-32a) | *1 |
| 1-600 | Cl | S | CH₂CH₃ | NHS(O)₂CF₃ | 249-250 |
| 1-601 | Cl | O | CH₂Ph | CH(CH₃)CH₂SCH₃ | *1 |
| 1-602 | Cl | O | CH₂Ph | CH₂CH₂SCH₃ | *1 |
| 1-603 | Cl | O | CH₂[D1-37b{4,6-(OCH₃)₂}] | CH(CH₃)SCH₂CH₃ | *1 |
| 1-604 | Cl | O | CH₂Ph | CH(CH₃)SCH₂CH₃ | *1 |
| 1-605 | Cl | O | —CH₂CH₂N{CH(CF₃)OCH₂CF₃}— | | 92-94 |
| 1-606 | Cl | O | —CH₂CH₂N(CH₂SCH₃)— | | 101-103 |
| 1-607 | Cl | O | CH₂CH₃ | NHNHC(O)OC(CH₃)₃ | 185-187 |
| 1-608 | Cl | O | CH₂CH₃ | NHNH₂ 2HCl | 172-174 |

Compound No. 1-047 of the present invention is a mixture of Compounds Nos. 1-047a and 1-047b, and Compound No. 1-093 of the present invention is a mixture of Compounds Nos. 1-093a and 1-093b.

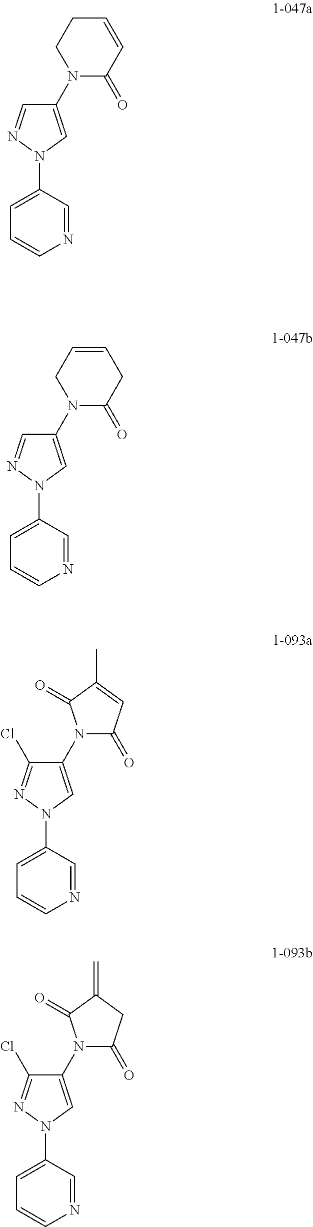 [SMILES: O=C1C=CCCN1c1cnn(-c2cccnc2)c1 ; O=C1CC=CCN1c1cnn(-c2cccnc2)c1 ; CC1=CC(=O)N(c2cn(-c3cccnc3)nc2Cl)C1=O ; C=C1CC(=O)N(c2cn(-c3cccnc3)nc2Cl)C1=O]

TABLE 5

| 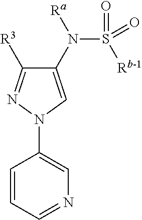 | | | | |
|---|---|---|---|---|
| No. | R³ | R^{a} | R^{b-1} | m.p.(° C.) |
| 2-001 | H | H | CH₃ | 140-150 |
| 2-002 | Cl | CH₃ | CH₂CH₂SCH₃ | 101-103 |
| 2-003 | Cl | CH₃ | CH═CH₂ | *1 |

TABLE 6

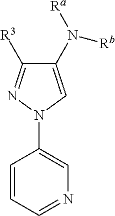

| No. | R³ | R^{a} | R^{b} | m.p.(° C.) |
|---|---|---|---|---|
| 3-001 | Cl | CH₃ | D1-51a | 125-127 |
| 3-002 | Me | | ═C(CH₃)OCH₃ | 90-92 |
| 3-003 | Me | | ═C(CH₃)OCH₂CH₃ | 56-58 |
| 3-004 | Me | | ═C(CH₃)N(CH₃)C(O)CH₂SCH₃ | 54-57 |
| 3-005 | Cl | | ═CHN(CH₃)₂ | 82-85 |
| 3-006 | Cl | | ═C(SCH₃)CH(CH₃)SCH₂CH₃ | *2(~2:1) |
| 3-007 | Cl | | D1-88b{X^{1a} = C(O)CH₂SCH₃} | 126-129 |
| 3-008 | Cl | H | D1-51a | 202-204 |
| 3-009 | H | | ═C(SCH₃)CH(CH₃)SCH₂CH₃ | *2(~2:1) |
| 3-010 | Cl | | ═CHN(OCH₃)C(O)CH(CH₃)CH₂SCH₃ | 58-59 |
| 3-011 | Cl | | ═CHN(OCH₃)C(O)Pr-c | 215-220 |
| 3-012 | Cl | | ═CHN(OCH₃)C(O)CH(CH₃)SCH₂CH₃ | *2(~77:23) |
| 3-013 | Cl | | ═CHN(OCH₃)C(O)CH₂CH₂SCH₃ | 99-100 |

TABLE 7

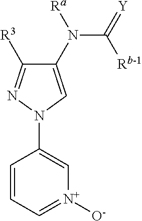

| No. | R³ | Y | R^{a} | R^{b-1} | m.p.(° C.) |
|---|---|---|---|---|---|
| 4-001 | Cl | O | CH₃ | CH(CH₃)CH₂S(O)₂CH₃ | 154-156 |
| 4-002 | Cl | O | CH₂CH₃ | OC(CH₃)₃ | 146-147 |
| 4-003 | Cl | O | CH₂CH₃ | N{CH₂S(O)₂CH₃}CH₂CF | *1 |

TABLE 8

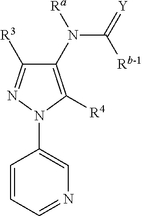

| No. | R³ | R⁴ | Y | R^{a} | R^{b-1} | m.p.(° C.) |
|---|---|---|---|---|---|---|
| 5-001 | H | CH₃ | O | H | OC(CH₃)₃ | 89-91 |
| 5-002 | H | CH₃ | O | CH₂CH₃ | OC(CH₃)₃ | 80-83 |
| 5-003 | H | CH₃ | O | CH₂CH₃ | CH(CH₃)CH₂SCH₃ | *1 |
| 5-004 | H | CH₃ | O | CH₂CH₃ | CH(CH₃)SCH₂CH₃ | *1 |
| 5-005 | H | Cl | O | H | OC(CH₃)₃ | *1 |
| 5-006 | H | Cl | O | H | OC(CH₃)₃ | 87-89 |
| 5-007 | Cl | SCH₂CH₃ | O | CH₂CH₃ | CH(CH₃)SCH₂CH₃ | *1 |

TABLE 9

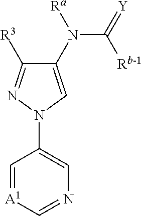

| No. | A¹ | R³ | R^{a} | R^{b-1} | m.p.(° C.) |
|---|---|---|---|---|---|
| 6-001 | N | Cl | CH₂CH₃ | CH(CH₃)SCH₂CH₃ | 154-156 |
| 6-002 | C—CH₃ | Cl | CH₂CH₃ | CH(CH₃)SCH₂CH₃ | *1 |
| 6-003 | C—OCH₃ | Cl | CH₂CH₃ | CH(CH₃)SCH₂CH₃ | *1 |
| 6-004 | C—F | Cl | H | OC(CH₃)₃ | 199-201 |
| 6-005 | C—F | Cl | CH₂CH₃ | OC(CH₃)₃ | 108-110 |
| 6-006 | C—CN | Cl | CH₂CH₃ | CH(CH₃)SCH₂CH₃ | 94-98 |
| 6-007 | C—CF₃ | Cl | CH₂CH₃ | CH(CH₃)SCH₂CH₃ | 100-102 |
| 6-008 | C—F | Cl | CH₂CH₃ | CH(CH₃)SCH₂CH₃ | 83-84 |
| 6-009 | C—F | Cl | CH₂CH₃ | CH(CH₃)CH₂SCH₃ | 80-81 |
| 6-010 | C—NH₂ | H | CH₃ | c-Pr | *1 |

TABLE 10

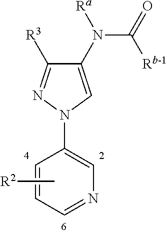

| No. | R² | R³ | R^{a} | R^{b-1} | m.p.(° C.) |
|---|---|---|---|---|---|
| 7-001 | 6-CH₃ | H | CH₃ | c-Pr | 198-199 |
| 7-002 | 6-CH₃ | Cl | CH₂CH₃ | CH(CH₃)SCH₂CH₃ | 57-60 |
| 7-003 | 4-CH₃ | Cl | CH₂CH₃ | CH(CH₃)SCH₂CH₃ | *1 |
| 7-004 | 2-CH₃ | Cl | CH₂CH₃ | CH(CH₃)SCH₂CH₃ | *1 |

TABLE 11

| No. | Proton NMR chemical shift (in CDCl₃): σ(ppm) |
|---|---|
| 1-018; | 8.93 (d, J = 2.7 Hz, 1H), 8.56 (dd, J = 4.8, 1.2 Hz, 1H), 8.01 (ddd, J = 8.4, 2.7, 1.2 Hz, 1H), 7.90 (s, 1H), |
| | 7.41 (dd, J = 8.4, 4.8 Hz, 1H), 3.22 (s, 3H), 2.77 (t, J = 7.2 Hz, 2H), 2.43 (t, J = 7.2 Hz, 2H), 2.27 (s, 3H), |
| | 2.04 (s, 3H) |
| 1-019; | 8.94 (d, J = 2.4 Hz, 1H), 8.56 (dd, J = 1.2, 4.8 Hz, 1H), 8.02 (ddd, J = 1.2, 2.4, 8.1 Hz, 1H), 7.86 (s, 1H), |
| | 7.42 (dd, J = 4.8, 8.1 Hz, 1H), 3.74-3.62 (m, 2H), 2.77 (t, J = 7.2 Hz, 2H), 2.39 (t, J = 7.2 Hz, 2H), 2.26 (s, |
| | 3H), 2.04 (s, 3H), 1.14 (t, J = 7.2 Hz, 3H) |
| 1-021; | 9.02-8.89 (m, 1H), 8.51 (dd, J = 1.2, 4.8 Hz, 1H), 8.30 (brs, 1H), 8.01 (ddd, J = 1.2, 2.7, 9.0 Hz, 1H), |
| | 7.72 (s, 1H), 7.37 (dd, J = 4.8, 9.0 Hz, 1H), 3.28 (s, 3H), 1.51 (s, 9H) |
| 1-022; | 9.01-8.92 (m, 1H), 8.51 (dd, J = 1.2, 4.8 Hz, 1H), 8.30 (brs, 1H), 8.01 (ddd, J = 1.2, 2.7, 9.0 Hz, 1H), |
| | 7.68 (s, 1H), 7.37 (dd, J = 4.8, 9.0 Hz, 1H), 3.71 (q, J = 7.2 Hz, 2H), 1.58 (s, 9H), 1.25 (t, J = 7.2 Hz, 3H) |
| 1-023; | 8.93 (d, J = 2.7 Hz, 1H), 8.55 (dd, J = 1.2, 4.8 Hz, 1H), 8.01 (ddd, J = 1.2, 2.7, 8.1 Hz, 1H), 7.92 (s, 1H), |
| | 7.41 (dd, J = 4.8, 8.1 Hz, 1H), 3.23 (s, 3H), 2.89-2.71 (m, 1H), 2.85 (d, J = 12.3 Hz, 1H), 2.42 (dd, J = 4.5, |
| | 12.3 Hz, 1H), 2.30 (s, 3H), 1.99 (s, 3H), 1.12 (d, J = 6.6 Hz, 3H) |
| 1-026; | 9.02 (d, J = 2.7 Hz, 1H), 8.58 (s, 1H), 8.54 (dd, J = 1.5, 4.8 Hz, 1H), 8.00-8.10 (m, 1H), 7.77 (s, 1H), |
| | 7.40 (dd, J = 4.8, 8.4 Hz, 1H), 3.83 (t, J = 7.2 Hz, 2H), 2.60 (t, J = 8.1 Hz, 2H), 2.20-2.35 (m, 2H) |
| 1-031; | 8.99-8.91 (m, 1H), 8.56 (d, J = 4.8 Hz, 1H), 8.02 (ddd, J = 1.2, 2.4, 8.1 Hz, 1H), 7.90 (s, 1H), 7.41 (dd, |
| | J = 4.8, 8.1 Hz, 1H), 3.90-3.40 (m, 2H), 2.87 (dd, J = 9.3, 12.3 Hz, 1H), 2.79-2.62 (m, 1H), 2.41 (dd, J = 5.1, |
| | 12.3 Hz, 1H), 2.29 (s, 3H), 1.99 (s, 3H), 1.12 (t, J = 6.3 Hz, 3H)1.11 (d, J = 6.6 Hz, 3H) |
| 1-033; | 8.94 (d, J = 2.4 Hz, 1H), 8.62 (dd, J = 1.2, 4.8 Hz, 1H), 8.04 (ddd, J = 1.2, 2.4, 8.4 Hz, 1H), 7.95 (s, 1H), |
| | 7.46 (dd, J = 4.8, 8.4 Hz, 1H), 3.71 (q, J = 7.2 Hz, 2H), 2.79 (dd, J = 7.2, 7.5 Hz, 2H), 2.44 (dd, J = 7.2, 7.5 Hz, |
| | 2H), 2.06 (s, 3H), 1.16 (t, J = 7.2 Hz, 3H) |
| 1-037; | 8.90 (d, J = 2.4 Hz, 1H), 8.56 (dd, J = 4.8, 1.5 Hz, 1H), 8.00 (ddd, J = 8.1, 2.4, 1.5 Hz, 1H), 7.90 (s, 1H), |
| | 7.40 (dd, J = 8.1, 4.8 Hz, 1H), 3.22 (s, 3H), 1.46 (s, 9H) |
| 1-038; | 8.94 (d, J = 2.7 Hz, 1H), 8.63 (dd, J = 4.5, 1.2 Hz, 1H), 8.04 (ddd, J = 8.1, 2.7, 1.2 Hz, 1H), 8.00 (s, 1H), |
| | 7.46 (dd, J = 8.1, 4.5 Hz, 1H), 3.25 (s, 3H), 2.80 (t, J = 7.2 Hz, 2H), 2.48 (t, J = 7.2 Hz, 2H), 2.07 (s, 3H) |
| 1-039; | 8.95 (d, J = 2.4 Hz, 1H), 8.63 (d, J = 4.5 Hz, 1H), 8.04 (ddd, J = 8.1, 2.4, 1.2 Hz, 1H), 8.04 (s, 1H), |
| | 7.46 (dd, J = 8.1, 4.5 Hz, 1H), 3.27 (s, 3H), 2.74-2.90 (m, 2H), 2.48 (dd, J = 12.6, 4.8 Hz, 1H), 2.03 (s, 3H), |
| | 1.17 (d, J = 6.3 Hz, 3H) |
| 1-040; | 8.95 (d, J = 2.4 Hz, 1H), 8.61 (dd, J = 1.8, 4.8 Hz, 1H), 8.02 (ddd, J = 1.2, 2.4, 8.4 Hz, 1H), 8.02 (s, 1H), |
| | 7.44 (dd, J = 4.8, 8.4 Hz, 1H), 3.76 (td, J = 7.2, 13.8 Hz, 1H), 3.67 (td, J = 7.2, 13.8 Hz, 1H), 3.14 (td, J = 7.8, |
| | 12.9 Hz, 1H), 2.86 (td, J = 6.3, 12.9 Hz, 1H), 2.72-2.60 (m, 2H), 2.58 (s, 3H), 1.16 (t, J = 7.2 Hz, 3H) |
| 1-042; | (the isomer contained in a ratio of 3)8.97 (d, J = 2.1 Hz, 1H), 8.63-8.58 (m, 1H), |
| | 8.08-7.99 (m, 1H), 7.96 (s, 1H), 7.69 (s, 1H), 7.44 (dd, J = 4.8, 8.1 Hz, 1H), 3.27 (s, 3H), 2.78 (t, J = 7.2 Hz, 2H), |
| | 2.53 (t, J = 7.2 Hz, 2H), 2.05 (s, 3H) |
| | (the isomer contained in a ratio of 2)9.02-8.97 (m, 1H), 8.61 (s, 1H), 8.53 (d, J = 3.9 Hz, 1H), |
| | 8.08-7.99 (m, 1H), 7.77 (s, 1H), 7.38 (dd, J = 4.8, 8.1 Hz, 1H), 3.43 (s, 3H), 2.94-2.80 (m, 4H), |
| | 2.18 (s, 3H) |
| 1-043; | (the isomer contained in a ratio of 3)8.97 (d, J = 2.4 Hz, 1H), 8.59 (d, J = 4.8 Hz, 1H), |
| | 8.08-7.99 (m, 1H), 8.01 (s, 1H), 7.72 (s, 1H), 7.44 (dd, J = 4.8, 8.1 Hz, 1H), 3.29 (s, 3H), 3.06-2.82 (m, 2H), |
| | 2.48-2.40 (m, 1H), 2.01 (s, 3H), 1.12 (d, J = 6.3 Hz, 3H) |
| | (the isomer contained in a ratio of 1)9.02-8.96 (m, 1H), 8.66 (s, 1H), 8.57-8.51 (m, 1H), |
| | 8.08-7.99 (m, 1H), 7.78 (s, 1H), 7.42-7.35 (m, 1H), 3.51 (s, 3H), 3.19-2.82 (m, 2H), 2.60 (dd, J = 6.0, |
| | 13.2 Hz, 1H), 2.14 (s, 3H), 1.30 (d, J = 6.9 Hz, 3H) |
| 1-044; | (the isomer contained in a ratio of 3)8.98 (d, J = 2.4 Hz, 1H), 8.60 (d, J = 4.8 Hz, 1H), |
| | 8.10-8.02 (m, 1H), 7.94 (s, 1H), 7.73 (s, 1H), 7.45 (dd, J = 4.8, 8.1 Hz, 1H), 3.71 (q, J = 7.2 Hz, 2H), 2.77 (t, J = 7.2 Hz, |
| | 2H), 2.48 (t, J = 7.2 Hz, 2H), 2.03 (s, 3H), 1.16 (t, J = 7.2 Hz, 3H) |
| | (the isomer contained in a ratio of 1)8.98 (d, J = 2.4 Hz, 1H), 8.62 (s, 1H), 8.53 (d, J = 4.8 Hz, |
| | 1H), 8.06-7.99 (m, 1H), 7.66 (s, 1H), 7.39 (dd, J = 4.8, 8.1 Hz, 1H), 3.80 (q, J = 7.2 Hz, 2H), |
| | 2.95-2.79 (m, 4H), 2.18 (s, 3H), 1.36 (t, J = 7.2 Hz, 3H) |
| 1-045; | (the isomer contained in a ratio of 5)8.98 (d, J = 2.4 Hz, 1H), 8.60 (dd, J = 1.2, 4.8 Hz, 1H), |
| | 8.06 (ddd, J = 1.2, 2.4, 8.4 Hz, 1H), 7.99 (s, 1H), 7.70 (s, 1H), 7.45 (dd, J = 4.8, 8.4 Hz, 1H), |
| | 3.94-3.58 (m, 2H), 2.93-2.75 (m, 2H), 2.49-2.35 (m, 1H), 2.01 (s, 3H), 1.16 (t, J = 7.2 Hz, 3H), 1.11 (d, J = 6.6 Hz, |
| | 3H) |
| | (the isomer contained in a ratio of 1)8.98 (d, J = 2.4 Hz, 1H), 8.66 (s, 1H), 8.58-8.54 (m, 1H), |
| | 8.06-7.98 (m, 1H), 7.74 (s, 1H), 7.45-7.37 (m, 1H), 3.94-3.58 (m, 2H), 3.16-2.94 (m, 2H), |
| | 2.67-2.57 (m, 1H), 2.14 (s, 3H), 1.40 (t, J = 7.2 Hz, 3H), 1.31 (d, J = 6.9 Hz, 3H) |
| 1-048; | 8.95 (d, J = 2.7 Hz, 1H), 8.61 (d, J = 4.8 Hz, 1H), 8.07-8.01 (m, 1H), 8.00 (s, 1H), 7.45 (dd, J = 4.8, 8.4 Hz, |
| | 1H), 3.92-3.71 (m, 1H), 3.69-3.50 (m, 1H), 2.85 (dd, J = 9.0, 12.6 Hz, 1H), 2.75-2.61 (m, 1H), |
| | 2.45 (dd, J = 5.1, 12.6 Hz, 1H), 2.01 (s, 3H), 1.16 (t, J = 7.8 Hz, 3H), 1.15 (d, J = 6.9 Hz, 3H) |
| 1-049; | (the isomer contained in a ratio of 10)9.02 (d, J = 2.7 Hz, 1H), 8.59 (dd, J = 1.2, 4.8 Hz, 1H), |
| | 8.22 (s, 1H), 8.06-7.98 (m, 1H), 7.40 (dd, J = 4.8, 8.1 Hz, 1H), 4.18-3.87 (m, 1H), 3.52-3.07 (m, 3H), |
| | 2.74-2.51 (m, 1H), 2.58 (s, 3H), 1.18 (d, J = 6.9 Hz, 3H), 1.15 (t, J = 7.2 Hz, 3H) |
| | (the isomer contained in a ratio of 7)8.98 (d, J = 2.7 Hz, 1H), 8.61 (dd, J = 1.2, 4.8 Hz, 1H), |
| | 8.15 (s, 1H), 8.06-7.98 (m, 1H), 7.43 (dd, J = 4.8, 8.1 Hz, 1H), 4.18-3.87 (m, 1H), 3.52-3.07 (m, 3H), |
| | 2.74-2.51 (m, 1H), 2.58 (s, 3H), 1.25 (d, J = 6.9 Hz, 3H), 1.20 (t, J = 7.2 Hz, 3H) |
| 1-050; | 9.01 (d, J = 2.7 Hz, 1H), 8.61 (dd, J = 1.2, 4.8 Hz, 1H), 8.18 (s, 1H), 8.00 (ddd, J = 1.2, 2.7, 8.1 Hz, 1H), |
| | 7.43 (dd, J = 4.8, 8.1 Hz, 1H), 4.17-4.01 (m, 1H), 3.81 (dd, J = 10.8, 13.5 Hz, 1H), 3.39-3.19 (m, 2H), |
| | 2.93 (s, 3H), 2.87 (dd, J = 2.7, 13.5 Hz, 1H), 1.20 (d, J = 7.2 Hz, 3H), 1.16 (t, J = 7.2 Hz, 3H) |
| 1-051; | 8.96 (d, J = 2.4 Hz, 1H), 8.63 (dd, J = 4.8, 1.2 Hz, 1H), 8.07 (s, 1H), 8.03 (ddd, J = 8.4, 2.4, 1.2 Hz, 1H), |
| | 7.46 (dd, J = 8.4, 4.8 Hz, 1H), 3.27 (s, 3H), 3.11-3.21 (m, 1H), 2.84-2.93 (m, 1H), 2.66-2.76 (m, 2H), |
| | 2.60 (s, 3H) |
| 1-052; | 8.96 (d, J = 2.4 Hz, 1H), 8.64 (d, J = 4.8 Hz, 1H), 8.06 (s, 1H), 8.04 (ddd, J = 8.4, 2.4, 1.5 Hz, 1H), |
| | 7.47 (dd, J = 8.4, 4.8 Hz, 1H), 3.43 (t, J = 7.2 Hz, 2H), 3.27 (s, 3H), 2.97 (s, 3H), 2.75 (t, J = 7.2 Hz, 2H) |
| 1-053; | (the isomer contained in a ratio of 4)8.99-9.03 (m, 1H), 8.60 (d, J = 4.5 Hz, 1H), 8.28 (s, 1H), |
| | 8.00 (dd, J = 8.1, 1.8 Hz, 1H), 7.41 (dd, J = 8.1, 4.5 Hz, 1H), 3.30 (s, 3H), 3.13-3.21 (m, 2H), |
| | 2.57-2.62 (m, 1H), 2.61 (s, 3H), 1.21 (d, J = 6.9 Hz, 3H) |
| | (the isomer contained in a ratio of 1)8.99-9.03 (m, 1H), 8.60 (d, J = 4.5 Hz, 1H), 8.20 (s, 1H), |
| | 8.00 (dd, J = 8.1, 1.8 Hz, 1H), 7.41 (dd, J = 8.1, 4.5 Hz, 1H), 3.26 (s, 3H), 3.13-3.21 (m, 2H), |
| | 2.57-2.62 (m, 1H), 2.61 (s, 3H), 1.21 (d, J = 6.9 Hz, 3H) |
| 1-054; | 9.00 (d, J = 2.7 Hz, 1H), 8.62 (dd, J = 4.5, 1.2 Hz, 1H), 8.22 (s, 1H), 8.00 (ddd, J = 8.1, 2.7, 1.2 Hz, 1H), |
| | 7.43 (dd, J = 8.1, 4.5 Hz, 1H), 3.83 (dd, J = 13.8, 10.5 Hz, 1H), 3.33-3.40 (m, 1H), 3.28 (s, 3H), |
| | 2.95 (s, 3H), 2.89 (dd, J = 13.8, 3.0 Hz, 1H), 1.16 (d, J = 6.9 Hz, 3H) |
| 1-059; | 9.20-8.90 (m, 1H), 8.70-8.45 (m, 1H), 8.20 (d, J = 9.0 Hz, 2H), 8.04 (d, J = 9.0 Hz, 2H), 8.03 (s, 1H), |
| | 7.60-7.40 (m, 1H), 7.25-7.20 (m, 1H), 3.74 (q, J = 6.9 Hz, 2H), 2.36 (s, 3H), 1.28 (t, J = 6.9 Hz, 3H) |
| 1-060; | 8.92 (d, J = 3.0 Hz, 1H), 8.51 (dd, J = 4.8 Hz, 1.8 Hz, 1H), 7.99 (ddd, J = 8.4 Hz, 3.0 Hz, 1.8 Hz, 1H), |
| | 7.95 (s, 1H), 7.38 (dd, J = 8.4 Hz, 3.0 Hz, 1H), 5.06 (brs, 1H), 3.65 (q, J = 7.2 Hz, 2H), 3.39 (q, J = 7.2 Hz, 2H), |
| | 2.60 (t, J = 7.2 Hz, 2H), 2.28 (s, 3H), 2.57 (s, 3H), 1.26 (t, J = 7.2 Hz, 3H) |
| 1-063; | 8.95 (d, J = 2.7 Hz, 1H), 8.62 (dd, J = 4.8, 1.2 Hz, 1H), 8.05 (ddd, J = 8.4, 2.7, 1.2 Hz, 1H), 7.96 (s, 1H), |
| | 7.46 (dd, J = 8.1, 4.8 Hz, 1H), 3.61 (m, 2H), 2.79 (m, 2H), 2.45 (m, 2H), 2.06 (s, 3H), 1.57 (m, 2H), |
| | 0.93 (t, J = 7.2 Hz, 3H) |
| 1-064; | 8.94 (d, J = 2.7 Hz, 1H), 8.63 (dd, J = 4.5, 1.2 Hz, 1H), 8.04 (ddd, J = 8.7, 2.4, 1.2 Hz, 1H), 7.93 (s, 1H), |
| | 7.46 (dd, J = 8.7, 4.5 Hz, 1H), 5.92-5.79 (m, 1H), 5.21-5.12 (m, 2H), 4.26 (brs, 2H), 2.81 (t, J = 7.5 Hz, |
| | 2H), 2.49 (t, J = 7.5 Hz, 2H), 2.07 (s, 3H) |
| 1-065; | 8.95 (d, J = 2.7 Hz, 1H), 8.62 (dd, J = 4.8, 1.5 Hz, 1H), 8.05 (ddd, J = 8.4, 2.7, 1.5 Hz, 1H), 7.95 (s, 1H), |
| | 7.46 (dd, J = 8.4, 4.8 Hz, 1H), 3.65-3.79 (m, 2H), 3.18-3.34 (m, 1H), 2.48 (dd, J = 15.9, 6.3 Hz, 1H), |
| | 2.26 (dd, J = 15.9, 7.8 Hz, 1H), 2.06 (s, 3H), 1.28 (d.J = 6.9 Hz, 3H), 1.17 (t, J = 7.2 Hz, 3H) |
| 1-066; | 8.96 (d, J = 2.7 Hz, 1H), 8.63 (dd, J = 4.5, 1.2 Hz, 1H), 8.07-8.01 (m, 1H), 8.01 (s, 1H), 7.46 (dd, J = 8.1, |
| | 4.5 Hz, 1H), 3.76 (brs, 1H), 3.50 (brs, 1H), 2.86 (dd, J = 12.6, 9.0 Hz, 1H), 2.74-2.70 (m, 1H), 2.47 (dd, |
| | J = 12.6, 5.1 Hz, 1H), 2.02 (s, 3H), 1.58 (tq, J = 7.8, 7.8 Hz, 2H), 1.16 (d, J = 6.6 Hz, 3H), 0.94 (t, J = 7.5 Hz, |
| | 3H) |
| 1-067; | 8.95 (d, J = 2.7 Hz, 1H), 8.62 (d, J = 4.8 Hz, 1H), 8.00-8.10 (m, 1H), 8.00 (s, 1H), 7.46 (dd, J = 4.8, 8.1 Hz, |
| | 1H), 4.74 (s, 2H), 4.06 (s, 2H), 3.72 (q, J = 7.5 Hz, 2H), 2.10 (s, 3H), 1.18 (t, J = 7.5 Hz, 3H) |
| 1-068; | 8.95 (d, J = 2.7 Hz, 1H), 8.65-8.59 (m, 1H), 8.07-8.00 (m, 1H), 7.99 (s, 1H), 7.45 (dd, J = 4.8, 8.1 Hz, 1H), |
| | 5.20-4.80 (m, 2H), 3.44 (s, 3H), 2.80 (t, J = 7.2 Hz, 2H), 2.54 (t, J = 7.2 Hz, 2H), 2.06 (s, 3H) |
| 1-076; | 8.94 (d, J = 2.4 Hz, 1H), 8.63-8.62 (m, 1H), 8.04 (ddd, J = 8.4, 2.7, 1.2 Hz, 1H), 7.99 (s, 1H), 7.46 (dd, |
| | J = 8.4, 4.8 Hz, 1H), 5.92-5.80 (m, 1H), 5.21-5.15 (m, 2H), 4.57 (brs, 1H), 4.00 (brs, 1H), 2.87 (m, 1H), |
| | 2.78-2.71 (m, 1H), 2.48 (m, 1H), 2.02 (s, 3H), 1.18 (d, J = 6.6 Hz, 3H) |
| 1-077; | 8.96 (d, J = 2.7 Hz, 1H), 8.63 (d, J = 4.5 Hz, 1H), 8.08-8.03 (m, 1H), 7.96 (d, J = 1.2 Hz, 1H), 7.46 (dd, J = 8.4, |
| | 4.8 Hz, 1H), 3.71 (q, J = 7.2 Hz, 2H), 3.11 (t, J = 7.2 Hz, 2H), 2.45 (t, J = 7.2 Hz, 2H), 2.28 (s, 3H), |
| | 1.16 (t, J = 7.2 Hz, 3H) |
| 1-078; | 8.96 (d, J = 2.1 Hz, 1H), 8.65-8.63 (m, 1H), 8.07 (ddd, J = 8.7, 3.0, 1.5 Hz, 1H), 7.90 (s, 1H), 7.47 (dd, |
| | J = 8.4, 4.8 Hz, 1H), 5.04 (qq, J = 6.6, 6.6 Hz, 1H), 2.82-2.72 (m, 2H), 2.46-2.29 (m, 2H), 2.06 (s, 3H), |
| | 1.56 (d, J = 6.6 Hz, 3H), 1.06 (d, J = 6.6 Hz, 3H) |
| 1-079; | 8.95 (d, J = 2.7 Hz, 1H), 8.62 (d, J = 4.8 Hz, 1H), 8.08 (s, 1H), 8.00-8.10 (m, 1H), 7.45 (dd, J = 4.8, 8.1 Hz, |
| | 1H), 3.71 (q, J = 7.2 Hz, 2H), 3.08 (s, 2H), 2.22 (s, 3H), 1.18 (t, J = 7.2 Hz, 3H) |
| 1-083; | 8.95 (d, J = 2.4 Hz, 1H), 8.62 (dd, J = 1.5, 4.8 Hz, 1H), 8.00-8.10 (m, 1H), 7.96 (s, 1H), 7.45 (dd, J = 4.8, |
| | 8.1 Hz, 1H), 3.72 (q, J = 7.2 Hz, 2H), 2.70-3.15 (m, 5H), 2.15-2.30 (m, 2H), 1.16 (t, J = 7.2 Hz, 3H) |
| 1-085; | 8.95 (d, J = 2.7 Hz, 1H), 8.63 (dd, J = 4.8, 1.5 Hz, 1H), 8.05 (ddd, J = 8.1, 2.3, 1.2 Hz, 1H), 7.96 (s, 1H), |
| | 7.47 (dd, J = 8.1, 4.5 Hz, 1H), 3.72 (q, J = 7.2 Hz, 2H), 2.82 (t, J = 7.2 Hz, 2H), 2.45 (q, J = 7.2 Hz, 2H), |
| | 2.44 (t, J = 7.5 Hz, 2H), 1.22 (t, J = 7.5 Hz, 3H), 1.16 (t, J = 7.2 Hz, 3H) |
| 1-086; | 8.95 (d, J = 2.4 Hz, 1H), 8.61 (d, J = 4.8 Hz, 1H), 8.06-8.00 (m, 1H), 8.01 (s, 1H), 7.44 (dd, J = 4.8, 8.1 Hz, |
| | 1H), 5.60-4.40 (m, 2H), 3.46 (s, 3H), 2.91-2.70 (m, 2H), 2.52-2.44 (m, 1H), 2.00 (s, 3H), 1.20 (d, |
| | J = 6.3 Hz, 3H) |
| 1-087; | 8.95 (m, 1H), 8.56 (m, 1H), 8.05-7.95 (m, 2H), 7.45-7.35 (m, 1H), 3.20 (s, 3H), 3.20-3.25 (m, 1H), |
| | 2.90-2.80 (m, 1H), 2.80-2.65 (m, 2H), 2.59 (s, 3H), 2.28 (s, 3H) |
| 1-088; | 8.95 (m, 1H), 8.56 (m, 1H), 8.05-7.95 (m, 2H), 7.45-7.35 (m, 1H), 3.41 (t, J = 7.2 Hz, 2H), 3.24 (s, 3H), |
| | 2.96 (s, 3H), 2.69 (t, J = 7.2 Hz, 2H), 2.28 (s, 3H) |
| 1-095; | 8.93 (d, J = 1.5 Hz, 1H), 8.55 (dd, J = 4.5, 1.5 Hz, 1H), 8.03 (s, 1H), 7.99 (dd, J = 4.5, 1.5 Hz, 1H), |
| | 7.41 (dd, J = 8.1, 4.5 Hz, 1H), 3.43 (s, 3H), 3.09 (s, 2H), 2.30 (s, 3H), 2.24 (s, 3H) |
| 1-097; | 8.99 (d, J = 1.5 Hz, 1H), 8.63 (dd, J = 4.5, 1.5 Hz, 1H), 8.22 (s, 1H), 7.99 (ddd, J = 8.1, 4.5, 1.5 Hz, 1H), |
| | 7.44 (ddd, J = 8.1, 4.5, 1.5 Hz, 1H), 3.89 (s, 2H), 3.80-3.50 (m, 2H), 3.56 (m, 2H), 1.44 (t, J = 7.5 Hz, 3H), |
| | 1.20 (t, J = 7.5 Hz, 3H) |
| 1-098; | 8.95 (d, J = 2.7 Hz, 1H), 8.61 (d, J = 4.8 Hz, 1H), 8.00-8.10 (m, 1H), 8.01 (s, 1H), 7.45 (dd, J = 4.8, 8.1 Hz, |
| | 1H), 3.75 (q, J = 7.2 Hz, 2H), 2.30 (s, 3H), 1.20 (t, J = 7.2 Hz, 3H) |
| 1-102; | 8.94 (d, J = 2.4 Hz, 1H), 8.62 (d, J = 4.8 Hz, 1H), 8.13 (s, 1H), 7.95-8.10 (m, 1H), 7.35-7.55 (m, 1H), |
| | 3.26 (s, 3H), 3.12 (s, 2H), 2.23 (s, 3H) |
| 1-103; | 8.97 (d, J = 2.7 Hz, 1H), 8.65 (dd, J = 4.8, 0.9 Hz, 1H), 8.06-8.05 (m, 2H), 7.48 (dd, J = 8.1, 4.5 Hz, 1H), |
| | 4.61 (brs, 1H), 4.02 (brs, 1H), 3.10-2.57 (m, 3H), 2.01 (s, 3H), 1.32-1.24 (m, 6H) |
| 1-104; | 8.98 (d, J = 2.7 Hz, 1H), 8.62 (dd, J = 5.1, 1.5 Hz, 1H), 8.08 (s, 1H), 8.05 (ddd, J = 8.4, 2.4, 1.5 Hz, 1H), |
| | 7.46 (dd, J = 8.4, 5.1 Hz, 1H), 3.69-3.66 (m, 2H), 3.16 (m, 1H), 2.88 (m, 1H), 2.72-2.65 (m, 2H), |
| | 2.60 (s, 3H), 1.58 (m, 2H), 0.93 (t, J = 7.5 Hz, 3H) |
| 1-105; | 8.97 (d, J = 2.7 Hz, 1H), 8.64 (dd, J = 5.1, 0.9 Hz, 1H), 8.04 (ddd, J = 8.7, 2.7, 1.5 Hz, 1H), 8.02 (s, 1H), |
| | 7.46 (dd, J = 8.1, 4.5 Hz, 1H), 3.63 (brs, 1H), 3.43 (brs, 1H), 2.96 (s, 3H), 2.72 (m, 2H), 1.64-1.52 (m, |
| | 4H), 0.94 (t, J = 7.2 Hz, 3H) |
| 1-106; | 8.99 (d, J = 3.0 Hz, 1H), 8.56 (d, J = 5.1 Hz, 1H), 8.07 (dd, J = 6.0 Hz, 1.5 Hz, 1H), 8.06 (s, 1H), |
| | 7.50-7.40 (m, 1H), 3.69 (brs, 2H), 3.08 (s, 2H), 2.30 (s, 3H), 2.22 (s, 3H), 1.20-1.05 (m, 3H) |
| 1-107; | 8.94 (d, J = 1.5 Hz, 1H), 8.61 (dd, J = 4.8 Hz, 1.5 Hz, 1H), 8.07 (s, 1H), 8.02 (ddd, J = 8.7 Hz, 4.8 Hz, 1.5 Hz, |
| | 1H), 7.44 (dd, J = 8.7 Hz, 4.8 Hz, 1H), 3.69 (brs, 2H), 3.10 (s, 2H), 2.67 (q, J = 7.5 Hz, 2H), 1.25 (t, J = 7.5 Hz, |
| | 3H), 1.17 (t, J = 7.5 Hz, 3H) |
| 1-109; | 8.95 (d, J = 1.5 Hz, 1H), 8.62 (dd, J = 4.8, 1.5 Hz, 1H), 8.06 (s, 1H), 8.03 (ddd, J = 8.1, 4.8, 1.5 Hz, 1H), |
| | 7.46 (dd, J = 8.1, 4.8 Hz, 1H), 5.80-5.65 (m, 1H), 5.17 (dd, J = 16.8 Hz, J = 1.5 Hz, 1H), 5.11 (dd, J = 10.8, |
| | 1.5 Hz, 1H), 3.60 (q, J = 7.2 Hz, 2H), 3.26 (d, J = 7.2 Hz, 2H), 3.06 (s, 2H), 1.17 (t, J = 7.2 Hz, 3H) |
| 1-110; | 8.96 (d, J = 1.5 Hz, 1H), 8.55 (dd, J = 4.8, 1.5 Hz, 1H), 8.04 (ddd, J = 8.4, 4.8, 1.5 Hz, 1H), 7.98 (s, 1H), |
| | 7.42 (dd, J = 8.4, 4.8 Hz, 1H), 3.69 (brs, 2H), 3.20-3.05 (m, 1H), 2.95-2.80 (m, 1H), 2.70-2.60 (m, 2H), |
| | 2.59 (s, 3H), 2.28 (s, 3H), 1.15 (t, J = 7.2 Hz, 3H) |
| 1-111; | 8.96 (d, J = 1.5 Hz, 1H), 8.55 (dd, J = 4.8, 1.5 Hz, 1H), 8.04 (ddd, J = 8.4, 4.8, 1.5 Hz, 1H), 7.97 (s, 1H), |
| | 7.43 (dd, J = 8.4, 4.8 Hz, 1H), 3.70 (brs, 2H), 3.41 (t, J = 7.2 Hz, 2H), 2.96 (s, 3H), 2.65 (t, J = 7.2 Hz, 2H), |
| | 2.28 (s, 3H), 1.15 (t, J = 7.2 Hz, 3H) |
| 1-112; | 8.98 (d, J = 2.7 Hz, 1H), 8.62 (d, J = 4.8 Hz, 1H), 8.20 (s, 1H), 7.95-8.10 (m, 1H), 7.40-7.55 (m, 1H), |
| | 3.87 (q, J = 7.2 Hz, 2H), 3.55-3.75 (m, 1H), 2.54 and 2.64 (s, 3H), 1.43 (d, J = 6.9 Hz, 3H), 1.18 and |
| | 1.21 (t, J = 7.2 Hz, 3H) |
| 1-113; | 8.97 (d, J = 2.7 Hz, 1H), 8.62 (dd, J = 1.5, 4.8 Hz, 1H), 8.19 (s, 1H), 7.95-8.05 (m, 1H), 7.44 (dd, J = 4.8, |
| | 9.0 Hz, 1H), 3.98 (q, J = 7.2 Hz, 2H), 3.45-3.60 (m, 1H), 3.00 (s, 3H), 1.65 (d, J = 7.2 Hz, 3H), 1.21 (t, J = 7.2 Hz, |
| | 3H) |
| 1-117; | 8.96 (d, J = 2.4 Hz, 1H), 8.63 (dd, J = 4.5, 1.5 Hz, 1H), 8.06 (ddd, J = 8.4, 2.7, 1.5 Hz, 1H), 7.99 (s, 1H), |
| | 7.47 (dd, J = 8.7, 4.5 Hz, 1H), 3.72 (q, J = 7.2 Hz, 2H), 2.79 (dt, J = 8.4, 6.6 Hz, 2H), 2.49 (t, J = 6.6 Hz, 2H), |
| | 1.68 (t, J = 8.4 Hz, 1H), 1.17 (t, J = 7.2 Hz, 3H) |
| 1-118; | 8.96 (d, J = 2.7 Hz, 1H), 8.61 (d, J = 3.6 Hz, 1H), 8.14 (s, 1H), 8.00-8.10 (m, 1H), 7.46 (dd, J = 4.8, 8.1 Hz, |
| | 1H), 3.63 (brs, 1H), 3.77 (brs, 1H), 3.30 (q, J = 6.9 Hz, 1H), 2.50-2.70 (m, 2H), 1.47 (d, J = 6.9 Hz, |
| | 3H), 1.17 (t, J = 7.5 Hz, 3H), 1.16 (t, J = 7.5 Hz, 3H) |
| 1-119; | 8.93 (d, J = 2.7 Hz, 1H), 8.51 (dd, J = 4.5 Hz, 1.5 Hz, 1H), 8.05-7.95 (m, 2H), 7.38 (dd, J = 8.4 Hz, 4.8 Hz, |
| | 1H), 3.50 (q, J = 7.2 Hz, 2H), 3.39 (t, J = 7.2 Hz, 2H), 2.64 (s, 3H), 2.54 (t, J = 7.2 Hz, 2H), 2.25 (s, 3H), |
| | 2.09 (s, 3H), 1.28 (t, J = 7.2 Hz, 3H) |
| 1-120; | (the isomer contained in a ratio of 1)8.95 (d, J = 2.7 Hz, 1H), 8.61 (d, J = 3.6 Hz, 1H), 8.11 (s, |
| | 1H), 8.10-8.00 (m, 1H), 7.45 (dd, J = 4.8, 8.1 Hz, 1H), 3.85-3.55 (brs, 2H), 2.93 (t, J = 7.2 Hz, 1H), |
| | 2.06 (s, 3H), 1.65 (qui, J = 7.2 Hz, 2H), 1.17 (t, J = 7.2 Hz, 3H), 0.94 (t, J = 7.5 Hz, 3H) |
| | (the isomer contained in a ratio of 1)8.95 (d, J = 2.7 Hz, 1H), 8.61 (d, J = 3.6 Hz, 1H), 8.11 (s, |
| | 1H), 8.10-8.00 (m, 1H), 7.45 (dd, J = 4.8, 8.1 Hz, 1H), 3.85-3.55 (brs, 2H), 2.93 (t, J = 7.2 Hz, 1H), |
| | 2.06 (s, 3H), 2.06 (qui, J = 7.2 Hz, 2H), 1.17 (t, J = 7.2 Hz, 3H), 0.94 (t, J = 7.5 Hz, 3H) |
| 1-121; | (the isomer contained in a ratio of 3)8.95 (d, J = 2.7 Hz, 1H), 8.63 (d, J = 3.6 Hz, 1H), 8.05 (s, |
| | 1H), 8.10-8.00 (m, 1H), 7.46 (dd, J = 8.1, 4.8 Hz, 1H), 5.86 (s, 1H), 3.75 (brs, 2H), 2.23 (d, J = 2.4 Hz, |
| | 3H), 1.20 (t, J = 7.2 Hz, 3H) |
| | (the isomer contained in a ratio of 2) |
| | 8.95 (d, J = 2.7 Hz, 1H), 8.63 (d, J = 3.6 Hz, 1H), 8.05 (s, 1H), 8.10-8.00 (m, 1H), 7.46 (dd, J = 8.1, 4.8 Hz, |
| | 1H), 5.69 (s, 1H), 3.75 (brs, 2H), 2.23 (d, J = 2.4 Hz, 3H), 1.20 (t, J = 7.2 Hz, 3H) |
| 1-122; | 8.94 (d, J = 2.7 Hz, 1H), 8.62 (dd, J = 4.8, 1.2 Hz, 1H), 8.04 (ddd, J = 8.4, 2.7, 1.5 Hz, 1H), 7.95 (s, 1H), |
| | 7.46 (dd, J = 8.4, 4.8 Hz, 1H), 3.71 (q, J = 7.2 Hz, 2H), 2.80 (m, 2H), 2.45 (m, 2H), 2.42 (m, 2H), |
| | 1.57 (m, 2H), 1.16 (t, J = 7.5 Hz, 3H), 0.94 (t, J = 7.5 Hz, 3H) |
| 1-123; | 8.94 (d, J = 2.4 Hz, 1H), 8.62 (dd, J = 4.8, 1.2 Hz, 1H), 8.04 (ddd, J = 8.4, 3.0, 1.5 Hz, 1H), 7.95 (s, 1H), |
| | 7.46 (dd, J = 7.8, 4.8 Hz, 1H), 3.71 (q, J = 7.2 Hz, 2H), 2.86 (m, 1H), 2.82 (t, J = 7.8 Hz, 2H), 2.42 (t, J = 7.8 Hz, |
| | 2H), 1.23 (d, J = 6.9 Hz, 6H), 1.16 (t, J = 7.2 Hz, 3H) |
| 1-124; | 8.95 (d, J = 2.7 Hz, 1H), 8.63 (dd, J = 4.5, 0.9 Hz, 1H), 8.04 (ddd, J = 8.4, 2.7, 1.2 Hz, 1H), 7.94 (s, 1H), |
| | 7.46 (dd, J = 8.4, 4.8 Hz, 1H), 3.71 (q, J = 7.2 Hz, 2H), 3.10 (q, J = 9.9 Hz, 2H), 2.96 (t, J = 6.9 Hz, 2H), |
| | 2.47 (t, J = 6.9 Hz, 2H), 1.17 (t, J = 7.2 Hz, 3H) |
| 1-125; | (the isomer contained in a ratio of 3)8.95 (d, J = 2.7 Hz, 1H), 8.61 (d, J = 4.8 Hz, 1H), 8.16 (s, |
| | 1H), 8.10-7.95 (m, 1H), 7.45 (dd, J = 4.8, 8.7 Hz, 1H), 6.17 (s, 1H), 2.72 (brs, 2H), 2.21 (s, 3H), |
| | 1.61 (s, 3H), 1.18 (t, J = 7.2 Hz, 3H) |
| | (the isomer contained in a ratio of 1)8.95 (d, J = 2.7 Hz, 1H), 8.61 (d, J = 4.8 Hz, 1H), 8.26 (s, |
| | 1H), 8.10-7.95 (m, 1H), 7.45 (dd, J = 4.8, 8.7 Hz, 1H), 6.17 (s, 1H), 2.75 (brs, 2H), 2.16 (s, 3H), |
| | 1.61 (s, 3H), 1.18 (t, J = 7.2 Hz, 3H) |
| 1-127; | (the isomer contained in a ratio of 3)8.97 (d, J = 2.7 Hz, 1H), 8.62 (q, J = 1.2 Hz, 1H), 8.27 (s, |
| | 1H), 8.02 (d, J = 8.1 Hz, 1H), 7.45 (dd, J = 8.1, 4.8 Hz, 1H), 5.94 (s, 1H), 3.12 (brs, 2H), 3.12 (s, 3H), |
| | 2.24 (s, 3H), 1.20 (d, J = 2.4 Hz, 3H) |
| | (the isomer contained in a ratio of 2)8.97 (d, J = 2.7 Hz, 1H), 8.62 (q, J = 1.2 Hz, 1H), 8.27 (s, |
| | 1H), 8.02 (d, J = 8.1 Hz, 1H), 7.45 (dd, J = 8.1, 4.8 Hz, 1H), 5.94 (s, 1H), 4.17 (brs, 2H), 3.12 (s, 3H), |
| | 2.24 (s, 3H), 1.20 (d, J = 2.4 Hz, 3H) |
| 1-134; | 8.95 (d, J = 1.5 Hz, 1H), 8.60 (d, J = 4.8 Hz, 1H), 8.14 (s, 1H), 8.04 (d, J = 8.4 Hz, 1H), 7.45 (dd, J = 8.4 Hz, |
| | 4.8 Hz, 1H), 3.21 (s, 3H), 2.72 (s, 2H), 2.14 (s, 3H), 1.21 (s, 6H) |
| 1-138; | 8.93 (d, J = 1.5 Hz, 1H), 8.53 (dd, J = 4.8 Hz, 1.5 Hz, 1H), 8.00 (ddd, J = 8.4 Hz, 4.8 Hz, 1.5 Hz, 1H), |
| | 7.95 (s, 1H), 7.41 (dd, J = 8.4 Hz, 4.8 Hz, 1H), 4.60 (brs, 1H), 3.25 (q, J = 7.2 Hz, 2H), 3.21 (s, 3H), 2.27 (s, |
| | 3H), 1.08 (t, J = 7.2 Hz, 3H) |
| 1-139; | 8.97 (d, J = 1.5 Hz, 1H), 8.60 (dd, J = 4.8 Hz, 1.5 Hz, 1H), 8.19 (s, 1H), 7.99 (dd, J = 8.4 Hz, 4.8, 1.5 Hz, |
| | 1H), 7.44 (dd, J = 8.4 Hz, 4.8 Hz, 1H), 3.47 (brs, 2H), 3.24 (s, 3H), 3.01 (s, 3H), 1.26 (s, 6H) |
| 1-142; | 8.89 (d, J = 2.1 Hz, 1H), 8.58 (dd, J = 4.8, 1.2 Hz, 1H), 8.04-7.95 (m, 1H), 8.01 (s, 1H), 7.43 (dd, J = 8.1, |
| | 4.8 Hz, 1H), 3.69 (s, 3H), 3.53 (s, 2H), 3.33 (s, 3H), 1.90 (s, 3H) |
| 1-143; | 8.90 (d, J = 2.4 Hz, 1H), 8.58 (dd, J = 4.8, 1.2 Hz, 1H), 8.04-7.95 (m, 1H), 7.98 (s, 1H), 7.43 (dd, J = 8.1, |
| | 4.8 Hz, 1H), 3.78 (q, J = 7.2 Hz, 2H), 3.68 (s, 3H), 3.52 (s, 2H), 1.90 (s, 3H), 1.22 (t, J = 7.2 Hz, 3H) |
| 1-144; | 8.96 (d, J = 2.1 Hz, 1H), 8.39 (d, J = 4.2 Hz, 1H), 8.12 (s, 1H), 8.10-8.00 (m, 1H), 7.47 (dd, J = 4.8, 3.5 Hz, |
| | 1H), 4.48 (brs, 2H), 2.80 (t, J = 7.5 Hz, 2H), 2.50 (t, J = 7.5 Hz, 2H), 2.26 (t, J = 2.7 Hz, 1H), 2.08 (s, 3H) |
| 1-146; | 8.97 (d, J = 2.7 Hz, 1H), 8.63 (d, J = 4.8 Hz, 1H), 8.16 (s, 1H), 8.05-8.00 (m, 1H), 7.46 (dd, J = 4.8, 3.9 Hz, |
| | 1H), 4.45 (brs, 2H), 3.43 (t, J = 7.0 Hz, 2H), 2.97 (s, 3H), 2.76 (t, J = 7.0 Hz, 2H), 2.29 (t, J = 2.4 Hz, 1H) |
| 1-147; | 8.97 (d, J = 2.7 Hz, 1H), 8.63 (d, J = 3.6 Hz, 1H), 8.15 (s, 1H), 8.06-8.03 (m, 1H), 7.46 (dd, J = 8.1, 4.8 Hz, |
| | 1H), 4.65 (brs, 2H), 2.90-2.65 (m, 2H), 2.55-2.45 (m, 1H), 2.25 (t, J = 2.4 Hz, 1H), 2.03 (s, 3H), |
| | 1.18 (d, J = 6.8 Hz, 3H) |
| 1-149; | 8.98 (d, J = 2.7 Hz, 1H), 8.63 (dd, J = 4.8, 1.5 Hz, 1H), 8.07 (ddd, J = 8.4, 2.7, 1.5 Hz, 1H), 8.04 (s, 1H), |
| | 7.47 (dd, J = 8.4, 4.8 Hz, 1H), 3.71 (q, J = 7.2 Hz, 2H), 1.97 (s, 3H), 1.17 (t, J = 7.2 Hz, 3H) |
| 1-151; | 8.95 (d, J = 2.4 Hz, 1H), 8.61 (dd, J = 4.8, 1.5 Hz, 1H), 8.18 (ddd, J = 8.1, 2.4, 1.5 Hz, 1H), 7.92 (s, 1H), |
| | 7.45 (dd, J = 8.4, 4.8 Hz, 1H), 3.70 (q, J = 7.2 Hz, 2H), 2.12 (t, J = 7.2 Hz, 2H), 1.70-1.59 (m, 2H), |
| | 1.15 (t, J = 7.2 Hz, 3H), 0.89 (t, J = 7.5 Hz, 3H) |
| 1-153; | 8.95 (d, J = 2.7 Hz, 1H), 8.63 (dd, J = 4.8, 1.5 Hz, 1H), 8.05 (ddd, J = 8.4, 2.7, 1.5 Hz, 1H), 7.97 (s, 1H), |
| | 7.46 (dd, J = 8.4, 4.8 Hz, 1H), 3.73 (t, J = 7.5 Hz, 2H), 2.54 (t, J = 7.2 Hz, 2H), 2.10 (s, 3H), 1.97 (s, 3H), |
| | 1.95-1.75 (m, 2H) |
| 1-155; | 8.96 (s, 1H), 8.63 (d, J = 4.7 Hz, 1H), 8.03 (s, 1H), 8.05-8.00 (m, 1H), 7.46 (dd, J = 8.3, 4.8 Hz, 1H), |
| | 3.95 (q, J = 9.2 Hz, 2H), 3.73 (q, J = 7.1 Hz, 2H), 3.56 (t, J = 5.7 Hz, 2H), 2.75 (t, J = 6.3 Hz, 2H), 1.19 (t, 7.1 Hz, |
| | 3H) |
| 1-156; | 8.95 (d, J = 2.4 Hz, 1H), 8.62 (dd, J = 4.8, 1.5 Hz, 1H), 8.14 (s, 1H), 8.04 (ddd, J = 8.4, 2.4, 1.5 Hz, 1H), |
| | 7.45 (dd, J = 8.4, 4.8 Hz, 1H), 4.00-3.70 (m, 1H), 3.70-3.40 (m, 1H), 3.36-3.24 (m, 1H), 3.21 (dd, J = 12.9, |
| | 9.9 Hz, 1H), 2.71 (dd, J = 12.9, 4.2 Hz, 1H), 2.11 (s, 3H), 2.10 (s, 3H), 1.19 (t, J = 7.2 Hz, 3H) |
| 1-157; | 8.91 (d, 2.4 Hz, 1H), 8.59 (dd, J = 4.8, 1.2 Hz, 1H), 8.05-8.00 (m, 1H), 7.99 (s, 1H), 7.43 (dd, J = 8.0, |
| | 4.8 Hz, 1H), 3.93 (q, 7.1 Hz, 2H), 3.80 (q, J = 7.1 Hz, 2H), 3.56 (s, 2H), 1.94 (s, 3H), 1.25 (t, 7.1 Hz, 3H), |
| | 1.02 (t, 7.1 Hz, 3H) |
| 1-159; | (the isomer contained in a ratio of 3)8.92 (d, J = 3.0 Hz, 1H), 8.61 (dd, J = 4.8, 1.2 Hz, 1H), |
| | 8.04 (s, 1H), 8.10-7.90 (m, 1H), 7.43 (dd, J = 8.1, 4.8 Hz, 1H), 5.20 (s, 2H), 3.77 (q, J = 7.2 Hz, 2H), |
| | 3.34 (s, 2H), 1.91 (s, 3H), 1.21 (t, J = 7.2 Hz, 3H) |
| | (the isomer contained in a ratio of 2)8.92 (d, J = 3.0 Hz, 1H), 8.61 (dd, J = 4.8, 1.2 Hz, 1H), |
| | 8.04 (s, 1H), 8.10-7.90 (m, 1H), 7.43 (dd, J = 8.1, 4.8 Hz, 1H), 5.14 (s, 2H), 3.77 (q, J = 7.2 Hz, 2H), |
| | 3.34 (s, 2H), 1.91 (s, 3H), 1.21 (t, J = 7.2 Hz, 3H) |
| 1-160; | (the isomer contained in a ratio of 3)8.95 (d, J = 2.7 Hz, 1H), 8.63 (dd, J = 4.8, 1.2 Hz, 1H), |
| | 8.05 (s, 1H), 8.10-7.90 (m, 1H), 7.46 (dd, J = 8.1, 4.8 Hz, 1H), 3.93 (t, J = 6.6 Hz, 1H), 3.82 (brs, 2H), |
| | 3.32 (s, 3H), 2.90-2.65 (m, 2H), 2.06 (s, 3H), 1.19 (t, J = 7.2 Hz, 3H) |
| | (the isomer contained in a ratio of 2)8.95 (d, J = 2.7 Hz, 1H), 8.63 (dd, J = 4.8, 1.2 Hz, 1H), |
| | 8.05 (s, 1H), 8.10-7.90 (m, 1H), 7.46 (dd, J = 8.1, 4.8 Hz, 1H), 3.82 (brs, 2H), 3.72 (t, J = 6.6 Hz, 1H), |
| | 3.32 (s, 3H), 2.90-2.65 (m, 2H), 2.06 (s, 3H), 1.19 (t, J = 7.2 Hz, 3H) |
| 1-161; | 8.92 (d, J = 2.7 Hz, 1H), 8.59 (dd, J = 4.8, 1.2 Hz, 1H), 8.05 (s, 1H), 8.00 (ddd, J = 8.1, 2.7, 1.2 Hz, 1H), |
| | 7.43 (dd, J = 8.1, 4.8 Hz, 1H), 5.17 (s, 2H), 3.77 (q, J = 7.2 Hz, 2H), 3.39 (s, 2H), 3.37 (q, J = 7.2 Hz, 2H), |
| | 1.20 (t, J = 7.2 Hz, 3H), 1.18 (t, J = 7.2 Hz, 3H) |
| 1-162; | (the isomer contained in a ratio of 4)8.97 (d, J = 2.4 Hz, 1H), 8.58 (dd, J = 4.8, 1.2 Hz, 1H), |
| | 8.18 (s, 1H), 8.00 (ddd, J = 8.4, 2.4, 1.2 Hz, 1H), 7.41 (dd, J = 8.4, 4.8 Hz, 1H), 4.05 (d, J = 12.4 Hz, 1H), |
| | 3.92 (d, J = 12.4 Hz, 1H)3.82-3.72 (m, 2H), 3.75 (s, 3H), 2.58 (s, 3H), 1.22 (t, J = 7.2 Hz, 3H) |
| | (the isomer contained in a ratio of 1)9.00-8.95 (m, 1H), 8.61-8.55 (m, 1H), 8.40 (s, 1H), |
| | 8.06-8.00 (m, 1H), 7.45-7.37 (m, 1H), 4.05 (d, J = 12.4 Hz, 1H), 3.92 (d, J = 12.4 Hz, 1H)3.82-3.72 (m, |
| | 2H), 3.79 (s, 3H), 2.69 (s, 3H), 1.17 (t, J = 7.2 Hz, 3H) |
| 1-166; | 8.90 (m, 2H), 8.52 (d, J = 4.8 Hz, 1H), 8.25 (bs, 1H), 7.95-8.05 (m, 1H), 7.70 (s, 1H), 7.38 (m, 1H), |
| | 3.32 (s, 2H), 2.29 (s, 3H), 1.54 (s, 9H) |
| 1-167; | 8.94 (d, J = 2.7 Hz, 1H), 8.62 (dd, J = 4.8, 1.5 Hz, 1H), 8.17 (s, 1H), 8.03 (ddd, J = 8.1, 2.7, 1.5 Hz, 1H), |
| | 7.45 (dd, J = 8.1, 4.8 Hz, 1H), 3.42-3.34 (m, 1H), 3.32-3.17 (m, 1H), 3.29 (s, 3H), 2.73 (dd, J = 13.2, |
| | 4.5 Hz, 1H), 2.12 (s, 3H), 2.10 (s, 3H) |
| 1-169; | 8.89 (d, J = 2.4 Hz, 1H), 8.53 (dd, J = 4.8 Hz, 1.5 Hz, 1H), 8.08 (s, 1H), 7.97 (ddd, J = 9.0 Hz, 2.4 Hz, 1.5 Hz, |
| | 1H), 7.39 (dd, J = 9.0 Hz, 4.8 Hz, 1H), 3.49 (s, 2H), 3.43 (q, J = 6.6 Hz, 2H), 3.30 (s, 3H), 2.34 (s, 3H), |
| | 2.13 (s, 3H), 1.11 (t, J = 6.6 Hz, 3H) |
| 1-172; | 8.90 (d, J = 2.7 Hz, 1H), 8.59 (dd, J = 4.8, 1.2 Hz, 1H), 8.05-8.00 (m, 1H), 7.98 (s, 1H), 7.43 (dd, J = 8.3, |
| | 4.8 Hz, 1H), 4.55 (sep, J = 6.3 Hz, 1H), 3.79 (q, J = 6.8 Hz, 2H), 3.57 (s, 2H), 1.95 (s, 3H), 1.23 (t, J = 6.8 Hz, |
| | 3H), 1.01 (d, J = 6.3 Hz, 6H) |
| 1-173; | 8.92 (d, 2.4 Hz, 1H), 8.59 (dd, J = 4.8, 1.2 Hz, 1H), 8.05-7.95 (m, 1H), 7.98 (s, 1H), 7.44 (dd, J = 8.3, |
| | 4.8 Hz, 1H), 4.25 (m, 2H), 3.85-3.75 (m, 2H), 3.53 (s, 2H), 1.95 (s, 3H), 1.40 (m, 2H), 1.23 (t, J = 7.1 Hz, |
| | 3H), 0.72 (t, J = 7.1 Hz, 3H) |
| 1-176; | 8.95 (d, J = 2.7 Hz, 1H), 8.63 (dd, J = 4.8, 1.5 Hz, 1H), 8.14 (s, 1H), 8.04 (ddd, J = 8.7, 2.7, 1.5 Hz, 1H), |
| | 7.46 (dd, J = 8.7, 4.8 Hz, 1H), 4.12 (t, J = 10.2 Hz, 1H), 3.61 (dd, J = 10.2, 4.2 Hz, 1H), 3.54-3.46 (m, 1H), |
| | 3.31 (s, 3H), 2.12 (s, 3H) |
| 1-177; | 8.94 (d, J = 2.4 Hz, 1H), 8.63 (dd, J = 4.2, 1.2 Hz, 1H), 8.03 (ddd, J = 8.1, 2.4, 1.2 Hz, 1H), 7.97 (s, 1H), |
| | 7.46 (dd, J = 8.1, 4.2 Hz, 1H), 3.25 (s, 3H), 3.11 (q, J = 9.9 Hz, 2H), 2.96 (t, J = 7.2 Hz, 2H), 2.50 (t, J = 7.2 Hz, |
| | 2H) |
| 1-178; | 8.95 (d, J = 2.7 Hz, 1H), 8.62 (dd, J = 4.8, 1.2 Hz, 1H), 8.05 (s, 1H), 8.02 (ddd, J = 8.7, 2.7, 1.2 Hz, 1H), |
| | 7.45 (dd, J = 8.7, 4.8 Hz, 1H), 3.59-3.40 (m, 2H), 3.32-3.24 (m, 1H), 3.22 (s, 3H), 3.14-3.06 (m, 1H), |
| | 2.75 (t, J = 6.6 Hz, 2H) |
| 1-179; | 8.95 (d, J = 2.4 Hz, 1H), 8.63 (dd, J = 4.8, 1.2 Hz, 1H), 8.04 (s, 1H), 8.02 (ddd, J = 8.7, 2.4, 1.2 Hz, 1H), |
| | 7.46 (dd, J = 8.7, 4.8 Hz, 1H), 3.97 (q, J = 9.0 Hz, 2H), 3.57 (t, J = 6.6 Hz, 2H), 3.27 (s, 3H), 2.79 (t, J = 6.6 Hz, |
| | 2H) |
| 1-181; | 8.95 (d, J = 2.4 Hz, 1H), 8.63 (dd, J = 4.8, 1.2 Hz, 1H), 8.04 (s, 1H), 8.05-8.00 (m, 1H), 7.47 (dd, J = 8.1, |
| | 4.8 Hz, 1H), 3.90-3.60 (m, 6H), 3.20-3.00 (m, 2H), 2.60-2.40 (m, 2H), 1.22 (t, J = 7.2 Hz, 3H), |
| | 1.19 (t, J = 7.2 Hz, 3H) |
| 1-182; | 8.97 (d, J = 2.7 Hz, 1H), 8.63 (dd, J = 4.8, 1.2 Hz, 1H), 8.13 (s, 1H), 8.05 (ddd, J = 8.1, 2.7, 1.2 Hz, 1H), |
| | 7.46 (dd, J = 8.1, 4.8 Hz, 1H), 3.78 (brs, 2H), 2.90-2.60 (m, 5H), 2.01 (s, 6H), 1.19 (t, J = 7.2 Hz, 3H) |
| 1-183; | 8.81 (d, J = 2.7 Hz, 1H), 8.59 (d, J = 4.8 Hz, 1H), 7.95-7.85 (m, 1H), 7.88 (s, 1H), 7.41 (dd, J = 8.3, 4.8 Hz, |
| | 1H), 7.15-7.10 (m, 5H), 4.96 (s, 2H), 3.76 (q, J = 7.1 Hz, 2H), 3.57 (s, 2H), 1.95 (s, 3H), 1.23 (t, J = 7.1 Hz, |
| | 3H) |
| 1-184; | 8.92 (d, J = 2.7 Hz, 1H), 8.59 (d, J = 4.8 Hz, 1H), 8.05-7.95 (m, 1H), 7.99 (s, 1H), 7.44 (dd, J = 8.0, 4.8 Hz, |
| | 1H), 3.87 (t, J = 6.5 Hz, 2H), 3.80 (q, J = 7.1 Hz, 2H), 3.55 (s, 2H), 1.95 (s, 3H), 1.40 (tt, J = 7.1, 6.5 Hz, |
| | 2H), 1.23 (t, J = 7.1 Hz, 3H), 1.19 (sxt, J = 7.1 Hz, 2H), 0.75 (t, J = 7.1 Hz, 3H) |
| 1-185; | 8.96 (d, J = 2.7 Hz, 1H), 8.64 (dd, J = 4.8, 1.2 Hz, 1H), 8.16 (s, 1H), 8.01 (ddd, J = 8.1, 2.7, 1.2 Hz, 1H), |
| | 7.46 (dd, J = 8.1, 4.8 Hz, 1H), 3.58 (dd, J = 7.8, 7.2 Hz, 1H), 3.31 (s, 3H), 3.14 (dd, J = 14.4, 7.8 Hz, 1H), |
| | 2.97 (dd, J = 14.4, 7.2 Hz, 1H), 2.56 (q, J = 7.5 Hz, 2H), 1.23 (t, J = 7.5 Hz, 3H) |
| 1-186; | 8.95 (d, J = 2.7 Hz, 1H), 8.63 (d, J = 4.8 Hz, 1H), 8.10-8.00 (ddd, J = 8.1, 2.7, 1.8 Hz, 1H), 8.05 (s, 1H), |
| | 7.46 (dd, J = 8.1, 4.8 Hz, 1H), 3.85 (t, J = 7.5 Hz, 2H), 2.70 (t, J = 7.5 Hz, 2H), 2.13 (s, 3H), 1.98 (s, 3H) |
| 1-190; | 8.91 (d, J = 2.4 Hz, 1H), 8.59 (dd, J = 4.8, 1.5 Hz, 1H), 7.95 (ddd, J = 8.4, 2.4, 1.5 Hz, 1H), 7.81 (s, 1H), |
| | 7.44 (dd, J = 8.4, 4.8 Hz, 1H), 4.02 (q, J = 9.0 Hz, 2H), 3.16 (s, 3H), 2.75 (s, 3H) |
| 1-191; | 9.00-8.90 (m, 1H), 8.65-8.55 (m, 1H), 8.00 (dd, J = 8.1, 1.5 Hz, 1H), 7.90 (s, 1H), 7.50-7.40 (m, 1H), |
| | 3.70 (s, 3H), 3.33 (s, 3H), 1.95 (s, 3H) |
| 1-193; | 8.88 (d, J = 2.4 Hz, 1H), 8.52 (d, J = 4.5 Hz, 1H), 8.00-7.95 (m, 1H), 7.94 (s, 1H), 7.39 (dd, J = 8.4, 4.5 Hz, |
| | 1H), 3.68 (s, 3H), 3.51 (s, 2H), 3.30 (s, 3H), 2.34 (s, 3H), 1.82 (s, 3H) |
| 1-195; | 8.97 (d, J = 2.7 Hz, 1H), 8.65 (dd, J = 4.8, 1.2 Hz, 1H), 8.12 (s, 1H), 8.02 (ddd, J = 8.1, 2.7, 1.2 Hz, 1H), |
| | 7.46 (dd, J = 8.1, 4.8 Hz, 1H), 4.00-3.41 (m, 3H), 3.14 (dd, J = 13.5, 7.5 Hz, 1H)2.97 (dd, J = 13.5, 7.2 Hz, |
| | 1H), 2.97 (q, J = 7.5 Hz, 2H), 1.23 (t, J = 7.5 Hz, 3H), 1.21 (t, J = 7.5 Hz, 3H) |
| 1-197; | (the isomer contained in a ratio of 10)8.97 (d, J = 2.1 Hz, 1H), 8.56-8.53 (m, 1H), 8.25 (brs, |
| | 1H), 8.04-8.00 (m, 1H), 7.81 (s, 1H), 7.39 (dd, J = 8.4, 4.8 Hz, 1H), 3.64-3.45 (m, 1H), 3.17 (s, 3H), |
| | 2.10 (s, 3H), 1.59 (s, 9H), 1.49 (d, J = 7.2 Hz, 3H) |
| | (the isomer contained in a ratio of 7)8.97 (d, J = 2.1 Hz, 1H), 8.56-8.53 (m, 1H), 8.25 (brs, 1H), |
| | 8.04-8.00 (m, 1H), 7.54 (s, 1H), 7.39 (dd, J = 8.4, 4.8 Hz, 1H), 3.64-3.45 (m, 1H), 3.26 (s, 3H), |
| | 2.11 (s, 3H), 1.56 (s, 9H), 1.42 (d, J = 7.2 Hz, 3H) |
| 1-198; | 8.96 (d, J = 2.7 Hz, 1H), 8.63 (dd, J = 4.8, 1.2 Hz, 1H), 8.06 (ddd, J = 8.4, 2.7, 1.2 Hz, 1H), 7.93 (s, 1H), |
| | 7.47 (dd, J = 8.4, 4.8 Hz, 1H), 3.72 (q, J = 7.2 Hz, 2H), 2.80 (t, J = 7.5 Hz, 2H), 2.44 (t, J = 7.5 Hz, 2H), |
| | 2.07 (s, 3H), 1.17 (t, J = 7.2 Hz, 3H) |
| 1-199; | (the isomer contained in a ratio of 3)8.98 (d, J = 2.7 Hz, 1H), 8.62 (dd, J = 4.8, 1.2 Hz, 1H), |
| | 8.06 (s, 1H), 8.10-8.00 (m, 1H), 7.43 (dd, J = 8.1, 4.8 Hz, 1H), 4.50-4.35 (m, 1H), 4.10-3.80 (m, 1H), |
| | 3.75-3.50 (m, 1H), 3.34 (s, 3H), 3.25-3.05 (m, 1H), 2.90-3.30 (m, 1H), 2.61 (s, 3H), 1.19 (t, J = 7.2 Hz, |
| | 3H) |
| | (the isomer contained in a ratio of 1)9.01 (d, J = 2.7 Hz, 1H), 8.62 (dd, J = 4.8, 1.2 Hz, 1H), |
| | 8.18 (s, 1H), 8.10-8.00 (m, 1H), 7.44 (dd, J = 8.1, 4.8 Hz, 1H), 4.50-4.35 (m, 1H), 4.10-3.80 (m, 1H), |
| | 3.75-3.50 (m, 1H), 3.30 (s, 3H), 3.25-3.05 (m, 1H), 2.90-3.30 (m, 1H), 2.64 (s, 3H), 1.20 (t, J = 7.2 Hz, |
| | 3H) |
| 1-201; | 8.96 (d, J = 2.7 Hz, 1H), 8.63 (dd, J = 4.8, 1.2 Hz, 1H), 8.07 (s, 1H), 8.03 (ddd, J = 8.1, 2.7, 1.2 Hz, 1H), |
| | 7.44 (dd, J = 8.1, 4.8 Hz, 1H), 3.90-3.50 (brs, 2H), 3.45 (q, J = 6.9 Hz, 1H), 3.25 (q, J = 9.9 Hz, 2H), |
| | 1.48 (d, J = 6.9 Hz, 3H), 1.17 (t, J = 7.2 Hz, 3H) |
| 1-202; | 8.97 (d, J = 2.7 Hz, 1H), 8.63 (dd, J = 4.8, 1.5 Hz, 1H), 8.06 (ddd, J = 8.4, 2.7, 1.5 Hz, 1H), 7.98 (s, 1H), |
| | 7.46 (dd, J = 8.4, 4.8 Hz, 1H), 4.10-3.30 (m, 2H), 2.86 (dd, J = 12.6, 8.7 Hz, 1H), 2.75-2.55 (m, 1H) |
| | 2.47 (dd, J = 12.6, 5.4 Hz, 1H), 2.02 (s, 3H), 1.18 (t, J = 6.9 Hz, 3H), 1.20-1.10 (m, 3H) |
| 1-203; | 8.97 (d, J = 2.7 Hz, 1H), 8.63 (dd, J = 4.8, 1.5 Hz, 1H), 8.06 (ddd, J = 8.4, 2.7, 1.5 Hz, 1H), 8.02 (s, 1H), |
| | 7.46 (dd, J = 8.4, 4.8 Hz, 1H), 3.85-3.60 (m, 2H), 3.15 (dt, J = 13.5, 7.2 Hz, 1H), 2.95-2.80 (m, 1H) |
| | 2.75-2.60 (m, 2H), 2.59 (s, 3H), 1.18 (t, J = 7.2 Hz, 3H) |
| 1-204; | 8.97 (d, J = 3.0 Hz, 1H), 8.64 (dd, J = 4.8, 1.5 Hz, 1H), 8.06 (ddd, J = 8.7, 3.0, 1.5 Hz, 1H), 7.99 (s, 1H), |
| | 7.46 (dd, J = 8.7, 4.8 Hz, 1H), 3.80-3.65 (m, 2H), 3.50-3.35 (m, 2H), 2.96 (s, 3H), 2.71 (t, J = 6.9 Hz, 2H), |
| | 1.18 (t, J = 7.2 Hz, 3H) |
| 1-205; | (the isomer contained in a ratio of 1)9.05 (d, J = 2.7 Hz, 1H), 8.61 (dd, J = 4.8, 1.5 Hz, 1H), |
| | 8.21 (s, 1H), 8.04 (ddd, J = 8.1, 2.7, 1.5 Hz, 1H), 7.44 (dd, J = 8.1, 4.8 Hz, 1H), 4.30-3.25 (m, 2H), |
| | 4.10-3.25 (m, 1H), 3.25-3.10 (m, 2H)2.59 (s, 3H), 1.27 (d, J = 6.9 Hz, 3H), 1.18 (t, J = 7.2 Hz, 3H) |
| | (the isomer contained in a ratio of 1)9.00 (d, J = 2.7 Hz, 1H), 8.60 (dd, J = 4.8, 1.5 Hz, 1H), |
| | 8.15 (s, 1H), 8.03 (ddd, J = 8.1, 2.7, 1.5 Hz, 1H), 7.41 (dd, J = 8.1, 4.8 Hz, 1H), 4.30-3.25 (m, 2H), |
| | 4.10-3.25 (m, 1H), 3.25-3.10 (m, 2H)2.59 (s, 3H), 1.23 (d, J = 6.9 Hz, 3H), 1.16 (t, J = 7.2 Hz, 3H) |
| 1-206; | 9.02 (d, J = 2.7 Hz, 1H), 8.62 (dd, J = 4.8, 1.2 Hz, 1H), 8.17 (s, 1H), 8.02 (ddd, J = 8.7, 2.7, 1.2 Hz, 1H), |
| | 7.43 (dd, J = 8.7, 4.8 Hz, 1H), 4.25-4.05 (m, 1H), 3.81 (dd, J = 13.5, 11.4 Hz, 1H)3.40-3.15 (m, 2H), |
| | 2.94 (s, 3H), 2.87 (dd, J = 13.5, 5.4 Hz, 1H), 1.22 (d, J = 6.9 Hz, 3H), 1.17 (t, J = 7.2 Hz, 3H) |
| 1-208; | 8.96 (d, J = 2.7 Hz, 1H), 8.58 (d, J = 4.8 Hz, 1H), 8.19 (s, 1H), 8.05-7.95 (m, 1H), 7.40 (dd, J = 8.3, 4.8 Hz, |
| | 1H), 4.48 (s, 2H), 4.10-4.00 (m, 4H), 2.59 (s, 3H), 1.21 (t, J = 7.1 Hz, 3H), 1.03 (t, J = 7.1 Hz, 3H) |
| 1-209; | 8.96 (d, J = 2.7 Hz, 1H), 8.59 (d, J = 4.8 Hz, 1H), 8.08 (s, 1H), 8.00-7.95 (m, 1H), 7.42 (dd, J = 8.1, 4.8 Hz, |
| | 1H), 4.48 (s, 2H), 4.05 (q, J = 7.1 Hz, 2H), 3.80-3.70 (m, 2H), 2.86 (s, 3H), 1.23 (t, J = 7.1 Hz, 3H), |
| | 1.05 (t, J = 7.1 Hz, 3H) |
| 1-210; | 8.96 (d, J = 2.7 Hz, 1H), 8.63 (dd, J = 4.8, 1.2 Hz, 1H), 8.07 (s, 1H), 8.03 (ddd, J = 8.1, 2.7, 1.2 Hz, 1H), |
| | 7.44 (dd, J = 8.1, 4.8 Hz, 1H), 3.90-3.50 (brs, 2H), 3.45 (q, J = 6.9 Hz, 1H), 3.25 (q, J = 9.9 Hz, 2H), |
| | 1.48 (d, J = 6.9 Hz, 3H), 1.17 (d, J = 7.2 Hz, 3H) |
| 1-211; | 8.90 (d, J = 2.7 Hz, 1H), 8.59 (d, J = 4.8 Hz, 1H), 8.01 (ddd, J = 8.1, 2.7, 1.5 Hz, 1H), 7.88 (s, 1H), |
| | 7.43 (dd, J = 8.1, 4.8 Hz, 1H), 3.78 (q, J = 7.2 Hz, 2H), 3.68 (s, 3H), 1.94 (s, 3H), 1.22 (t, J = 7.2 Hz, 3H) |
| 1-213; | 9.04 (d, J = 2.4 Hz, 1H), 8.62 (dd, J = 4.8, 1.5 Hz, 1H), 8.44 (s, 1H), 8.04 (ddd, J = 8.4, 2.4, 1.5 Hz, 1H), |
| | 7.45 (dd, J = 8.4, 4.8 Hz, 1H), 4.40-4.25 (m, 1H), 3.25-3.10 (m, 1H), 2.34 (d, J = 7.2 Hz, 1H), 1.56 (s, 3H), |
| | 1.33 (d, J = 7.2 Hz, 1H), 1.14 (t, J = 7.2 Hz, 3H) |
| 1-214; | 8.90 (d, J = 2.4 Hz, 1H), 8.59 (dd, J = 8.1, 1.5 Hz, 1H), 8.01 (ddd, J = 8.1, 2.4, 1.5 Hz, 1H), 7.83 (s, 1H), |
| | 7.43 (dd, J = 8.1, 4.8 Hz, 1H), 6.00-5.90 (m, 1H), 3.74 (q, J = 7.2 Hz, 2H), 2.25-2.05 (m, 2H), |
| | 2.05-1.90 (m, 2H), 1.80-1.40 (m, 4H), 1.18 (t, J = 7.2 Hz, 3H) |
| 1-215; | 8.94 (d, J = 2.4 Hz, 1H), 8.65-8.55 (m, 1H), 8.10-8.00 (m, 1H), 7.92 (s, 1H), 7.44 (dd, J = 8.4, 4.8 Hz, 1H), |
| | 5.70-5.65 (m, 2H), 3.80-3.60 (m, 2H), 2.55-2.25 (m, 2H), 2.15-1.90 (m, 3H), 1.90-1.70 (m, 2H), |
| | 1.15 (t, J = 7.2 Hz, 3H) |
| 1-216; | 8.74-8.60 (m, 1H), 8.60 (dd, J = 4.8, 1.5 Hz, 1H), 7.95-7.85 (m, 1H), 7.43 (dd, J = 8.4, 4.8 Hz, 1H), |
| | 7.26-7.24 (m, 4H), 7.09-7.03 (m, 2H), 3.90-3.50 (m, 3H), 3.30 (dd, J = 13.2, 9.0 Hz, 1H), 2.70 (dd, J = 13.2, |
| | 6.0 Hz, 1H), 2.05 (s, 3H), 1.12 (t, J = 7.2 Hz, 3H) |
| 1-217; | (the isomer contained in a ratio of 1)8.83-8.74 (m, 1H), 8.60 (dd, J = 4.5, 1.2 Hz, 1H), |
| | 8.05-7.95 (m, 1H), 7.42 (dd, J = 8.1, 4.5 Hz, 1H), 7.29-7.26 (m, 4H), 7.10-7.05 (m, 2H), 4.25-4.05 (m, 2H), |
| | 3.80-3.60 (m, 2H), 2.73 (dd, J = 12.0, 2.7 Hz, 1H), 2.55 (s, 3H), 1.12 (t, J = 7.2 Hz, 3H) |
| | (the isomer contained in a ratio of 1)8.83-8.74 (m, 1H), 8.60 (dd, J = 4.5, 1.2 Hz, 1H), |
| | 8.05-7.95 (m, 1H), 7.42 (dd, J = 8.1, 4.5 Hz, 1H), 7.29-7.26 (m, 4H), 7.10-7.05 (m, 2H), 4.25-4.05 (m, 2H), |
| | 3.80-3.60 (m, 1H), 3.46 (dd, J = 12.9, 5.4 Hz, 1H), 3.08 (dd, J = 12.9, 8.7 Hz, 1H), 2.62 (s, 3H), |
| | 1.15 (t, J = 7.2 Hz, 3H) |
| 1-218; | 9.00-8.88 (m, 1H), 8.62 (d, J = 3.9 Hz, 1H), 8.10-7.95 (m, 1H), 7.44 (dd, J = 9.0, 5.4 Hz, 1H), |
| | 7.30-7.26 (m, 4H), 7.08-7.05 (m, 2H), 4.40-4.00 (m, 4H), 3.16-3.10 (m, 1H), 2.89 (s, 3H), 1.14 (t, J = 7.2 Hz, 3H) |
| 1-219; | 8.98 (s, 1H), 8.61 (d, J = 4.8 Hz, 1H), 8.06 (d, J = 8.1 Hz, 1H), 8.01 (s, 1H), 7.44 (dd, J = 8.1, 4.8 Hz, 1H), |
| | 3.92-3.70 (m, 1H), 3.66-3.48 (m, 1H), 3.12 (dd, J = 13.2, 6.6 Hz, 1H)2.84 (dd, J = 13.2, 7.2 Hz, 1H), |
| | 2.64 (dqd, J = 7.2, 6.9, 6.6 Hz, 1H), 2.26 (s, 3H), 1.19-1.10 (m, 6H) |
| 1-221; | 8.91 (d, J = 2.7 Hz, 1H), 8.60 (d, J = 4.8, 0.9 Hz, 1H), 8.05-7.95 (m, 1H), 7.91 (s, 1H), 7.44 (dd, J = 8.3, |
| | 4.8 Hz, 1H), 3.78 (q, J = 7.1 Hz, 2H), 3.71 (s, 3H), 3.64 (s, 2H), 3.14 (q, J = 9.9 Hz, 2H), 1.23 (t, J = 7.1 Hz, |
| | 3H) |
| 1-222; | 8.91 (d, J = 2.7 Hz, 1H), 8.59 (dd, J = 4.8, 1.2 Hz, 1H), 8.05-7.95 (m, 1H), 7.97 (s, 1H), 7.43 (dd, J = 8.3, |
| | 4.8 Hz, 1H), 5.68 (ddt, J = 17.4, 10.5, 5.7 Hz, 1H), 5.11 (dd, J = 17.4, 1.2 Hz, 1H), 5.00 (dd, J = 10.5, 1.2 Hz, |
| | 1H), 4.37 (d, J = 5.7 Hz, 2H), 3.78 (q, J = 7.1 Hz, 2H), 3.57 (s, 2H), 1.96 (s, 3H), 1.23 (t, J = 7.1 Hz, |
| | 3H) |
| 1-223; | 8.92 (d, J = 2.7 Hz, 1H), 8.58 (dd, J = 4.8, 0.9 Hz, 1H), 8.05-7.95 (m, 1H), 8.00 (s, 1H), 7.43 (dd, J = 8.3, |
| | 4.8 Hz, 1H), 4.02 (t, J = 4.8 Hz, 2H), 3.79 (q, J = 7.1 Hz, 2H), 3.58 (s, 2H), 3.35 (t, J = 4.8 Hz, 2H), |
| | 3.14 (s, 3H), 1.93 (s, 3H), 1.23 (t, J = 7.1 Hz, 3H) |
| 1-225; | (the isomer contained in a ratio of 3)8.97 (d, J = 2.7 Hz, 1H), 8.62 (dd, J = 4.8, 1.2 Hz, 1H), |
| | 8.17 (s, 1H), 8.10-7.95 (m, 1H), 7.44 (dd, J = 8.1, 4.8 Hz, 1H), 3.91 (q, J = 6.9 Hz, 1H), 4.05-3.55 (m, 2H), |
| | 2.90-2.70 (m, 2H), 1.55-1.10 (m, 9H) |
| | (the isomer contained in a ratio of 1)8.97 (d, J = 2.7 Hz, 1H), 8.62 (dd, J = 4.8, 1.2 Hz, 1H), |
| | 8.14 (s, 1H), 8.10-7.95 (m, 1H), 7.44 (dd, J = 8.1, 4.8 Hz, 1H), 3.91 (q, J = 6.9 Hz, 1H), 4.05-3.55 (m, 2H), |
| | 2.60-2.45 (m, 2H), 1.55-1.10 (m, 9H) |
| 1-226; | 8.97 (d, J = 2.7 Hz, 1H), 8.62 (dd, J = 4.8, 1.2 Hz, 1H), 8.20 (s, 1H), 7.99 (ddd, J = 8.1, 2.4, 1.2 Hz, 1H), |
| | 7.43 (dd, J = 8.1, 4.8 Hz, 1H), 4.03 (q, J = 7.2 Hz, 1H), 4.10-3.35 (m, 2H), 3.45-3.10 (m, 2H), 1.64 (d, J = 7.2 Hz, |
| | 3H), 1.38 (t, J = 7.5 Hz, 3H), 1.21 (t, J = 7.2 Hz, 3H) |
| 1-227; | 8.91 (d, J = 2.4 Hz, 1H), 8.59 (dd, J = 4.8, 1.2 Hz, 1H), 7.96-7.89 (m, 1H), 7.91 (s, 1H), 7.42 (dd, J = 8.1, |
| | 4.8 Hz, 1H), 4.73 (s, 2H), 3.80 (s, 3H)3.36 (s, 3H) |
| 1-228; | 8.92 (d, J = 2.4 Hz, 1H), 8.59 (dd, J = 4.8, 1.2 Hz, 1H), 7.94 (ddd, J = 8.1, 2.4, 1.2 Hz, 1H), 7.88 (s, 1H), |
| | 7.42 (dd, J = 8.1, 4.8 Hz, 1H), 4.73 (s, 2H), 3.86-3.73 (m, 2H)3.81 (s, 3H)1.23 (t, J = 7.2 Hz, 3H) |
| 1-229; | (the isomer contained in a ratio of 3)9.00 (d, J = 2.7 Hz, 1H), 8.60 (dd, J = 4.8, 1.2 Hz, 1H), |
| | 8.17 (s, 1H), 8.10-7.95 (m, 1H), 7.50-7.35 (m, 1H), 4.20-3.90 (m, 1H), 3.80-3.15 (m, 5H), |
| | 2.95-2.75 (m, 1H), 1.30-1.10 (m, 6H) |
| | (the isomer contained in a ratio of 1)8.99 (d, J = 2.7 Hz, 1H), 8.60 (dd, J = 4.8, 1.2 Hz, 1H), |
| | 8.19 (s, 1H), 8.10-9.95 (m, 1H), 7.50-7.35 (m, 1H), 4.20-3.90 (m, 1H), 3.80-3.15 (m, 5H), |
| | 2.95-2.75 (m, 1H), 1.30-1.10 (m, 6H) |
| 1-230; | 8.99 (d, J = 2.4 Hz, 1H), 8.62 (dd, J = 4.8, 1.2 Hz, 1H), 8.13 (s, 1H), 8.00 (ddd, J = 8.1, 2.4, 1.2 Hz, 1H), |
| | 7.43 (dd, J = 8.1, 4.8 Hz, 1H), 4.20-3.90 (m, 3H), 3.90-3.70 (m, 1H), 3.40-3.20 (m, 2H), 3.20 (dd, J = 14.1, |
| | 2.7 Hz, 1H), 1.22 (d, J = 7.2 Hz, 3H), 1.17 (t, J = 7.2H, 3H) |
| 1-231; | 8.95 (d, J = 2.4 Hz, 1H), 8.63 (dd, J = 4.8, 1.2 Hz, 1H), 8.03 (ddd, J = 8.1, 2.4, 1.2 Hz, 1H), 8.00 (s, 1H), |
| | 7.46 (dd, J = 8.1, 4.8 Hz, 1H), 3.26 (s, 3H), 3.20-2.95 (m, 3H), 2.85-2.70 (m, 1H), 2.60-2.70 (m, 1H), |
| | 1.16 (d, J = 6.9 Hz, 3H) |
| 1-232; | 8.94 (d, J = 2.7 Hz, 1H), 8.63 (dd, J = 4.8, 1.2 Hz, 1H), 8.04 (ddd, J = 8.1, 2.7, 1.2 Hz, 1H), 8.01 (s, 1H), |
| | 7.46 (dd, J = 8.1, 4.8 Hz, 1H), 3.71 (q, J = 7.2 Hz, 2H), 3.32 (q, J = 9.9 Hz, 2H), 3.25 (s, 2H), 1.18 (t, J = 7.2 Hz, |
| | 3H) |
| 1-233; | 8.96 (d, J = 2.7 Hz, 1H), 8.59 (dd, J = 4.8, 1.2 Hz, 1H), 8.04 (s, 1H), 8.00 (ddd, J = 8.1, 2.7, 1.2 Hz, 1H), |
| | 7.43 (dd, J = 8.1, 4.8 Hz, 1H), 4.25-4.05 (m, 2H), 3.85-3.45 (m, 7H), 1.23 (t, J = 7.1 Hz, 3H) |
| 1-234; | 8.94 (d, J = 2.7 Hz, 1H), 8.58 (dd, J = 4.8, 1.2 Hz, 1H), 8.05-7.95 (m, 1H), 7.97 (s, 1H), 7.41 (dd, J = 8.1, |
| | 4.8 Hz, 1H), 3.88 (q, J = 9.9 Hz, 2H), 3.85-3.75 (m, 7H), 1.24 (t, J = 7.1 Hz, 3H) |
| 1-235; | 8.89 (d, J = 2.7 Hz, 1H), 8.59 (dd, J = 4.8, 1.2 Hz, 1H), 7.99 (ddd, J = 8.1, 2.7, 1.2 Hz, 1H), 7.93 (s, 1H), |
| | 7.43 (dd, J = 8.1, 4.8 Hz, 1H), 3.71 (s, 3H), 3.65 (s, 2H), 3.34 (s, 3H), 3.11 (q, J = 9.9 Hz, 2H) |
| 1-236; | 8.94 (d, J = 2.7 Hz, 1H), 8.59 (dd, J = 4.8, 1.2 Hz, 1H), 8.07 (s, 1H), 8.00-7.95 (m, 1H), 7.42 (dd, J = 8.1, |
| | 4.8 Hz, 1H), 4.25 (m, 2H), 3.80-3.30 (m, 8H) |
| 1-237; | 8.93 (d, J = 2.7 Hz, 1H), 8.58 (dd, J = 4.8, 1.2 Hz, 1H), 7.99 (s, 1H), 8.00-7.90 (m, 1H), 7.42 (dd, J = 8.1, |
| | 4.8 Hz, 1H), 4.59 (s, 2H), 3.95 (q, J = 8.7 Hz, 2H), 3.79 (s, 3H), 3.35 (s, 3H) |
| 1-240; | 8.94 (d, J = 2.4 Hz, 1H), 8.63 (dd, J = 4.8, 1.2 Hz, 1H), 8.08 (s, 1H), 8.01 (ddd, J = 8.1, 2.4, 1.2 Hz, 1H), |
| | 7.45 (dd, J = 8.1, 4.8 Hz, 1H), 3.53 (q, J = 7.2 Hz, 1H), 3.30-3.19 (m, 2H), 3.25 (s, 3H), 1.48 (d, J = 7.2 Hz, |
| | 3H) |
| 1-241; | 8.96 (d, J = 2.4 Hz, 1H), 8.64 (dd, J = 4.8, 1.2 Hz, 1H), 8.18 (s, 1H), 7.99 (ddd, J = 8.7, 2.4, 1.2 Hz, 1H), |
| | 7.45 (dd, J = 8.7, 4.8 Hz, 1H), 4.31-4.00 (m, 3H), 3.34 (s, 3H), 1.67 (d, J = 7.2 Hz, 3H) |
| 1-242; | 8.94 (d, J = 2.7 Hz, 1H), 8.63 (dd, J = 4.5, 1.5 Hz, 1H), 8.12 (s, 1H), 8.03 (ddd, J = 8.4, 2.7, 1.5 Hz, 1H), |
| | 7.46 (dd, J = 8.4, 4.5 Hz, 1H), 3.25 (s, 3H), 3.14 (s, 2H), 2.70 (q, J = 7.2 Hz, 2H), 1.27 (t, J = 7.2 Hz, 3H) |
| 1-243; | 8.97 (d, J = 2.4 Hz, 1H), 8.62 (dd, J = 4.8, 1.2 Hz, 1H), 8.22 (s, 1H), 8.00 (ddd, J = 8.1, 2.4, 1.2 Hz, 1H), |
| | 7.44 (dd, J = 8.1, 4.8 Hz, 1H), 3.69 (d, J = 13.5 Hz, 1H), 3.56 (d, J = 13.5 Hz, 1H), 3.30 (s, 3H), |
| | 3.03-2.88 (m, 2H), 1.35 (t, J = 7.5 Hz, 3H) |
| 1-245; | 8.98 (d, J = 2.7 Hz, 1H), 8.62 (dd, J = 4.8, 1.2 Hz, 1H), 8.12 (s, 1H), 8.05 (ddd, J = 8.1, 2.7, 1.2 Hz, 1H), |
| | 7.45 (dd, J = 8.1, 4.8 Hz, 1H), 4.28 (q, J = 7.2 Hz, 1H), 3.95-3.50 (m, 2H), 2.25 (s, 3H), 1.43 (d, J = 7.2 Hz, |
| | 3H), 1.16 (t, J = 7.2 Hz, 3H) |
| 1-246; | 8.95 (d, J = 2.4 Hz, 1H), 8.61 (dd, J = 4.8, 1.2 Hz, 1H), 8.11 (s, 1H), 8.03 (ddd, J = 8.1, 2.4, 1.2 Hz, 1H), |
| | 7.45 (dd, J = 8.1, 4.8 Hz, 1H), 3.95-3.50 (m, 2H), 3.28 (q, J = 6.9 Hz, 1H), 2.65-2.45 (m, 2H), 1.52 (t, J = 7.2 Hz, |
| | 2H), 1.46 (d, J = 6.9 Hz, 3H), 1.16 (t, J = 7.2 Hz, 3H), 0.94 (t, J = 7.2 Hz, 3H) |
| 1-248; | 8.95 (d, J = 2.4 Hz, 1H), 8.62 (dd, J = 4.8, 1.2 Hz, 1H), 8.10 (s, 1H), 8.03 (ddd, J = 8.1, 2.4, 1.2 Hz, 1H), |
| | 7.45 (dd, J = 8.1, 4.8 Hz, 1H), 3.90-3.50 (m, 2H), 3.34 (q, J = 6.9 Hz, 1H), 2.96 (qui, J = 6.9 Hz, 1H), |
| | 1.49 (d, J = 6.9 Hz, 3H), 1.25-1.05 (m, 9H) |
| 1-251; | 8.97 (d, J = 2.4 Hz, 1H), 8.59 (dd, J = 4.8, 1.2 Hz, 1H), 8.16 (s, 1H), 8.02 (ddd, J = 8.1, 2.4, 1.2 Hz, 1H), |
| | 7.44 (dd, J = 8.1, 4.8 Hz, 1H), 4.07 (q, J = 6.9H, 1H), 3.95-3.55 (m, 2H), 1.60 (d, J = 6.9 Hz, 3H), 1.18 (t, |
| | J = 7.2 Hz, 3H) |
| 1-252; | 8.97 (d, J = 2.4 Hz, 1H), 8.62 (dd, J = 4.8, 1.2 Hz, 1H), 8.14 (s, 1H), 8.03 (ddd, J = 8.1, 2.4, 1.2 Hz, 1H), |
| | 7.45 (dd, J = 8.1, 4.8 Hz, 1H), 3.95-3.30 (m, 5H), 1.54 (d, J = 6.9 Hz, 3H), 1.19 (t, J = 7.2 Hz, 3H) |
| 1-253; | (the isomer contained in a ratio of 54)8.98 (d, J = 2.4 Hz, 1H), 8.64 (dd, J = 4.8, 1.2 Hz, 1H), |
| | 8.10 (s, 1H), 8.04 (ddd, J = 8.1, 2.4, 1.2 Hz, 1H), 7.47 (dd, J = 8.1, 4.8 Hz, 1H), 4.35-3.55 (m, 4H), |
| | 3.55-3.30 (m, 1H), 1.54 (d, J = 6.9 Hz, 3H), 1.18 (t, J = 7.2 Hz, 3H) |
| | (the isomer contained in a ratio of 46)8.98 (d, J = 2.4 Hz, 1H), 8.61 (dd, J = 4.8, 1.2 Hz, 1H), |
| | 8.17 (s, 1H), 7.99 (ddd, J = 8.1, 2.4, 1.2 Hz, 1H), 7.43 (dd, J = 8.1, 4.8 Hz, 1H), 4.35-3.55 (m, 4H), |
| | 3.30-3.05 (m, 1H), 1.37 (d, J = 6.9 Hz, 3H), 1.22 (t, J = 7.2 Hz, 3H) |
| 1-256; | 8.97 (d, J = 2.4 Hz, 1H), 8.52 (dd, J = 4.8, 1.5 Hz, 1H), 8.21 (brs, 1H), 8.02 (ddd, J = 8.4, 2.4, 1.5 Hz, 1H), |
| | 7.66 (s, 1H), 7.38 (dd, J = 8.4, 4.8 Hz, 1H), 1.55-1.50 (m, 27H) |
| 1-257; | 8.97 (brs, 1H), 8.51 (brs, 1H), 8.22 (brs, 1H), 8.05-8.95 (m, 1H), 7.75 (s, 1H), 7.45-7.30 (m, 1H), |
| | 6.73 (brs, 1H), 1.60-1.1.50 (m, 18H) |
| 1-259; | 8.90 (d, J = 2.1 Hz, 1H), 8.59 (dd, J = 4.8, 1.2 Hz, 1H), 8.04-7.94 (m, 1H), 7.99 (s, 1H), 7.42 (dd, J = 8.1, |
| | 4.8 Hz, 1H), 3.69 (s, 3H), 3.56 (s, 2H), 3.33 (s, 3H), 2.33 (q, J = 7.2 Hz, 2H), 1.16 (t, J = 7.2 Hz, 3H) |
| 1-260; | (the isomer contained in a ratio of 3)8.96 (d, J = 2.4 Hz, 1H), 8.60-8.54 (m, 1H), 8.24 (s, 1H), |
| | 7.99 (ddd, J = 8.1, 2.4, 1.2 Hz, 1H), 7.44-7.37 (m, 1H), 3.98 (d, J = 12.6 Hz, 1H), 3.93 (d, J = 12.6 Hz, |
| | 1H), 3.74 (s, 3H), 3.32 (s, 3H), 2.95-2.68 (m, 2H), 1.32 (t, J = 7.2 Hz, 3H) |
| | (the isomer contained in a ratio of 2)8.99 (d, J = 2.7 Hz, 1H), 8.60-8.54 (m, 1H), 8.52 (s, 1H), |
| | 8.07-7.99 (m, 1H), 7.44-7.37 (m, 1H), 3.98 (d, J = 12.6 Hz, 1H), 3.93 (d, J = 12.6 Hz, 1H), 3.76 (s, 3H), |
| | 3.33 (s, 3H), 2.95-2.68 (m, 2H), 1.36 (t, J = 7.2 Hz, 3H) |
| 1-262; | 8.90 (d, J = 2.4 Hz, 1H), 8.59 (dd, J = 4.8, 1.2 Hz, 1H), 8.04-7.94 (m, 1H), 7.97 (s, 1H), 7.43 (dd, J = 8.1, |
| | 4.8 Hz, 1H), 3.78 (q, J = 7.2 Hz, 2H), 3.68 (s, 3H), 3.55 (s, 2H), 2.33 (q, J = 7.2 Hz, 2H), 1.22 (t, J = 7.2 Hz, |
| | 3H), 1.16 (t, J = 7.2 Hz, 3H) |
| 1-263; | (the isomer contained in a ratio of 4)8.97 (d, J = 2.4 Hz, 1H), 8.57 (dd, J = 4.8, 1.2 Hz, 1H), |
| | 8.20 (s, 1H), 8.00 (ddd, J = 8.4, 2.4, 1.2 Hz, 1H), 7.40 (dd, J = 8.4, 4.8 Hz, 1H), 3.98 (d, J = 12.3 Hz, 1H), |
| | 3.90 (d, J = 12.3 Hz, 1H), 3.83-3.69 (m, 2H), 3.74 (s, 3H), 2.95-2.64 (m, 2H), 1.25 (t, J = 7.2 Hz, 3H), |
| | 1.21 (t, J = 7.2 Hz, 3H) |
| | (the isomer contained in a ratio of 1)8.99 (d, J = 2.7 Hz, 1H), 8.60-8.54 (m, 1H), 8.46 (s, 1H), |
| | 8.07-8.00 (m, 1H), 7.46-7.37 (m, 1H), 3.98 (d, J = 12.3 Hz, 1H), 3.90 (d, J = 12.3 Hz, 1H), |
| | 3.83-3.69 (m, 2H), 3.74 (s, 3H), 2.95-2.64 (m, 2H), 1.36 (t, J = 7.2 Hz, 3H), 1.17 (t, J = 7.2 Hz, 3H) |
| 1-267; | 8.95 (d, J = 2.7 Hz, 1H), 8.58 (dd, J = 4.8, 1.5 Hz, 1H), 8.04 (s, 1H), 8.05-7.95 (m, 1H), 7.42 (dd, J = 8.3, |
| | 4.8 Hz, 1H), 4.93 (s, 2H), 3.81 (q, J = 7.1 Hz, 2H), 3.57 (s, 2H), 3.10 (s, 3H), 1.94 (s, 3H), 1.22 (t, J = 7.1 Hz, |
| | 3H) |
| 1-268; | 8.90 (d, J = 3.0 Hz, 1H), 8.59 (dd, J = 4.8, 1.5 Hz, 1H), 7.98 (s, 1H), 8.00-7.95 (m, 1H), 7.41 (dd, J = 8.3, |
| | 4.8 Hz, 1H), 3.94 (t, J = 8.3 Hz, 2H), 3.78 (q, J = 7.1 Hz, 2H), 3.55 (s, 2H), 1.97 (s, 3H), 1.23 (t, J = 7.1 Hz, |
| | 3H), 0.73 (t, J = 8.3 Hz, 2H), −0.075 (s, 9H) |
| 1-270; | (the isomer contained in a ratio of 54)8.96 (d, J = 3.0 Hz, 1H), 8.70-8.55 (m, 1H), 8.19 (s, 1H), |
| | 8.10-7.95 (m, 1H), 7.45 (dd, J = 8.7, 4.8 Hz, 1H), 4.10-3.90 (m, 1H), 3.32 (s, 3H), 2.65 (s, 3H), |
| | 1.43 (d, J = 6.9 Hz, 3H) |
| | (the isomer contained in a ratio of 46)8.96 (d, J = 3.0 Hz, 1H), 8.70-8.55 (m, 1H), 8.22 (s, 1H), |
| | 8.10-7.95 (m, 1H), 7.43 (dd, J = 8.7, 4.8 Hz, 1H), 4.10-3.90 (m, 1H), 3.29 (s, 3H), 2.55 (s, 3H), |
| | 1.38 (d, J = 6.9 Hz, 3H) |
| 1-273; | 8.90 (d, J = 2.4 Hz, 1H), 8.63-8.57 (m, 1H), 7.98 (ddd, J = 8.1, 2.4, 1.2 Hz, 1H), 7.92 (s, 1H), 7.43 (dd, |
| | J = 8.1, 4.5 Hz, 1H), 4.45 (s, 2H), 3.91 (q, J = 8.4 Hz, 2H), 3.76 (s, 3H), 3.33 (s, 3H) |
| 1-274; | 8.91 (d, J = 2.7 Hz, 1H), 8.60 (dd, J = 4.8, 1.2 Hz, 1H), 7.98 (ddd, J = 8.4, 2.7, 1.2 Hz, 1H), 7.90 (s, 1H), |
| | 7.43 (dd, J = 8.4, 4.8 Hz, 1H), 4.43 (s, 2H), 3.91 (q, J = 8.5 Hz, 2H), 3.78 (q, J = 7.2 Hz, 2H), 3.75 (s, 3H), |
| | 1.21 (t, J = 7.2 Hz, 3H) |
| 1-276; | 8.95 (d, J = 2.4 Hz, 1H), 8.61 (dd, J = 4.8, 1.2 Hz, 1H), 8.11 (s, 1H), 8.03 (ddd, J = 8.1, 2.4, 1.2 Hz, 1H), |
| | 7.44 (dd, J = 8.1, 4.8 Hz, 1H), 5.60-5.20 (m, 1H), 4.80-4.40 (m, 1H), 3.46 (s, 3H), 3.37 (q, J = 6.9 Hz, 1H), |
| | 2.66-2.51 (m, 2H), 1.50 (d, J = 6.9 Hz, 3H), 1.16 (t, J = 7.5 Hz, 3H) |
| 1-277; | 8.95 (d, J = 2.4 Hz, 1H), 8.62 (dd, J = 4.8, 1.2 Hz, 1H), 8.11 (s, 1H), 8.05 (ddd, J = 8.1, 2.4, 1.2 Hz, 1H), |
| | 7.46 (dd, J = 8.1, 4.8 Hz, 1H), 4.71 (d, J = 12.0 Hz, 1H), 4.55 (d, J = 12.0 Hz, 1H), 4.00-3.75 (m, 1H), |
| | 3.55 (q, J = 7.2 Hz, 2H), 3.27 (s, 3H), 1.49 (d, J = 6.9 Hz, 3H), 1.17 (t, J = 7.2 Hz, 3H) |
| 1-278; | 8.93 (d, J = 2.4 Hz, 1H), 8.58 (dd, J = 4.8, 1.2 Hz, 1H), 8.13 (s, 1H), 8.01 (ddd, J = 8.1, 2.4, 1.2 Hz, 1H), |
| | 7.43 (dd, J = 8.1, 4.8 Hz, 1H), 3.90-3.45 (m, 2H), 3.32 (q, J = 6.9 Hz, 1H), 2.55-2.35 (m, 2H), 1.44 (d, J = 6.9 Hz, |
| | 3H), 1.14 (t, J = 7.2 Hz, 3H), 0.90-0.70 (m, 1H), 0.60-0.30 (m, 2H), 0.30-0.60 (m, 2H) |
| 1-279; | 8.92 (d, J = 2.7 Hz, 1H), 8.61 (dd, J = 4.8, 1.5 Hz, 1H), 8.10-8.00 (m, 1H), 7.97 (s, 1H), 7.50-7.40 (m, 1H), |
| | 7.30-7.10 (m, 1H), 4.80-4.65 (m, 1H), 3.95-3.75 (m, 1H), 3.75-3.50 (m, 1H), 1.81 (m, 3H), |
| | 1.36 (t, J = 7.2 Hz, 3H), 1.25-1.10 (m, 3H) |
| 1-284; | 8.98 (d, J = 2.7 Hz, 1H), 8.65 (dd, J = 4.8, 1.2 Hz, 1H), 8.32 (s, 1H), 8.02 (ddd, J = 8.4, 2.7, 1.2 Hz, 1H), |
| | 7.47 (dd, J = 8.4, 4.8 Hz, 1H), 5.10-4.80 (m, 1H), 4.40-4.10 (m, 1H), 3.33 (q, J = 6.9 Hz, 1H), |
| | 2.67-2.53 (m, 2H), 1.49 (d, J = 6.9 Hz, 3H), 1.20 (t, J = 7.2 Hz, 3H) |
| 1-285; | 8.96 (d, J = 2.7 Hz, 1H), 8.63 (dd, J = 4.8, 1.2 Hz, 1H), 8.04 (s, 1H), 8.01 (ddd, J = 8.3, 2.7, 1.2 Hz, 1H), |
| | 7.45 (dd, J = 8.3, 4.8 Hz, 1H), 4.75-4.10 (m, 2H), 4.05 (s, 2H), 3.95-3.70 (m, 2H), 1.20 (t, J = 7.1 Hz, 3H) |
| 1-286; | 8.96 (d, J = 2.4 Hz, 1H), 8.62 (dd, J = 4.8, 1.2 Hz, 1H), 8.08 (s, 1H), 8.03 (ddd, J = 8.3, 2.4, 1.2 Hz, 1H), |
| | 7.41 (dd, J = 8.3, 4.8 Hz, 1H), 3.90-3.50 (m, 5H), 2.08 (s, 3H), 1.49 (d, J = 7.1 Hz, 3H), 1.17 (t, J = 7.1 Hz, |
| | 3H) |
| 1-288; | 8.93 (m, 1H), 8.63-8.59 (m, 1H), 8.05 (s, 1H), 8.02 (ddd, J = 8.1, 2.7, 1.2 Hz, 1H), 7.46 (dd, J = 8.1, 4.8 Hz, |
| | 1H), 3.32 (q, J = 9.9 Hz, 2H), 3.28 (s, 2H), 3.26 (s, 3H) |
| 1-289; | 8.95 (d, J = 2.4 Hz, 1H), 8.62 (dd, J = 4.8, 1.2 Hz, 1H), 8.22 (s, 1H), 8.04 (ddd, J = 8.1, 2.4, 1.2 Hz, 1H), |
| | 7.45 (dd, J = 8.1, 4.8 Hz, 1H), 4.97-4.60 (m, 1H), 4.30-3.95 (m, 1H), 3.39-3.27 (m, 1H), |
| | 2.66-2.52 (m, 2H), 2.25 (t, J = 2.1 Hz, 1H), 1.48 (d, J = 6.9 Hz, 3H), 1.17 (t, J = 7.2 Hz, 3H) |
| 1-291; | 8.95 (d, J = 2.4 Hz, 1H), 8.61 (dd, J = 4.8, 1.5 Hz, 1H), 8.10-8.00 (m, 2H), 7.44 (dd, J = 8.3, 2.4 Hz, 1H), |
| | 3.35-3.20 (m, 1H), 3.40-3.20 (m, 2H), 2.65-2.50 (m, 2H), 1.47 (d, J = 7.1 Hz, 3H), 1.17 (t, J = 7.1 Hz, |
| | 3H), 1.16 (t, J = 7.1 Hz, 3H) |
| 1-292; | 9.06 (d, J = 2.7 Hz, 1H), 8.62 (dd, J = 4.8, 1.2 Hz, 1H), 8.25 (s, 1H), 8.08 (ddd, J = 8.0, 2.7, 1.2 Hz, 1H), |
| | 7.44 (dd, J = 8.0, 4.8 Hz, 1H), 6.75 (brs, 1H), 3.75-3.65 (m, 2H), 3.54 (q, J = 7.1 Hz, 1H), 3.25 (d, J = 17.0 Hz, |
| | 1H), 3.18 (d, J = 17.0 Hz, 1H), 2.79 (d, J = 5.1 Hz, 3H), 1.44 (d, J = 7.1 Hz, 3H), 1.15 (t, J = 7.1 Hz, |
| | 3H) |
| 1-293; | 9.00 (d, J = 2.7 Hz, 1H), 8.61 (dd, J = 4.8, 1.2 Hz, 1H), 8.25 (s, 1H), 8.05 (ddd, J = 8.6, 2.7, 1.2 Hz, 1H), |
| | 7.44 (dd, J = 8.6, 4.8 Hz, 1H), 3.66 (s, 3H), 3.60-3.30 (m, 5H), 1.45 (d, J = 6.8 Hz, 3H), 1.16 (t, J = 7.2 Hz, |
| | 3H) |
| 1-300; | 8.94 (d, J = 2.7 Hz, 1H), 8.60 (dd, J = 4.8, 1.2 Hz, 1H), 8.31 (s, 1H), 8.00 (ddd, J = 8.4, 2.7, 1.2 Hz, 1H), |
| | 7.42 (dd, J = 8.4, 4.8 Hz, 1H), 4.80-4.50 (m, 1H), 4.30-3.80 (m, 1H), 4.19 (q, J = 7.2 Hz, 2H), 3.41 (q, J = 6.6 Hz, |
| | 1H), 2.66-2.49 (m, 2H), 1.46 (d, J = 6.6 Hz, 3H), 1.27 (t, J = 7.2 Hz, 3H), 1.17 (t, J = 7.2 Hz, 3H) |
| 1-302; | 8.88 (d, J = 2.4 Hz, 1H), 8.56-8.51 (m, 1H), 7.98 (ddd, J = 8.1, 2.4, 1.5 Hz, 1H), 7.93 (s, 1H), |
| | 7.42-7.34 (m, 1H), 4.82 (q, J = 6.9 Hz, 1H), 3.77 (s, 3H), 2.69-2.44 (m, 2H), 1.52 (d, J = 6.9 Hz, 3H), 1.20 (t, J = 7.2 Hz, |
| | 3H) |
| 1-303; | 8.95 (d, J = 2.7 Hz, 1H), 8.63 (dd, J = 4.8, 1.2 Hz, 1H), 8.05 (ddd, J = 8.1, 2.7, 1.2 Hz, 1H), 7.96 (s, 1H), |
| | 7.46 (dd, J = 8.1, 4.8 Hz, 1H), 3.98 (s, 3H), 3.70 (q, J = 7.2 Hz, 2H), 3.33 (s, 2H), 1.16 (t, J = 7.2 Hz, 3H) |
| 1-304; | 8.95 (d, J = 2.7 Hz, 1H), 8.68-8.61 (m, 1H), 8.15 (s, 1H), 8.00 (ddd, J = 8.1, 2.7, 1.2 Hz, 1H), 7.46 (dd, |
| | J = 8.1, 4.8 Hz, 1H), 4.42-4.20 (m, 2H), 4.08 (s, 2H), 3.33 (s, 3H) |
| 1-305; | 8.85-9.15 (m, 1H), 8.55-8.65 (m, 1H), 7.95-8.55 (m, 2H), 7.35-7.50 (m, 1H), 3.45-4.25 (m, 3H), |
| | 2.45-3.35 (m, 2H), 1.10-1.65 (m, 9H) |
| 1-307; | 8.96 (d, J = 2.4 Hz, 1H), 8.61 (dd, J = 4.8, 1.2 Hz, 1H), 8.17 (s, 1H), 8.04 (ddd, J = 8.1, 2.4, 1.2 Hz, 1H), |
| | 7.45 (dd, J = 8.1, 4.8 Hz, 1H), 3.55-3.90 (m, 2H), 1.45-1.50 (m, 2H), 1.20-1.30 (m, 2H), 1.17 (t, J = 7.2 Hz, |
| | 3H) |
| 1-308; | 8.92 (d, J = 2.4 Hz, 1H), 8.62 (dd, J = 4.8, 1.5 Hz, 1H), 8.05-7.95 (m, 2H), 7.45 (dd, J = 8.4, 4.8 Hz, 1H), |
| | 5.35 (brs, 1H), 3.80-3.75 (m, 2H), 3.26 (s, 3H), 1.42 (s, 9H) |
| 1-309; | 8.98-8.97 (m, 1H), 8.63-8.60 (m, 1H), 8.15 (s, 1H), 8.03-7.98 (m, 1H), 7.46-7.41 (m, 1H), |
| | 5.60-5.40 (m, 1H), 4.70-4.50 (m, 1H), 4.15-3.98 (m, 1H), 3.48 (s, 3H), 2.86-2.49 (m, 2H), 1.41 (d, J = 6.9 Hz, |
| | 3H), 1.38-1.24 (m, 3H) |
| 1-310; | 8.98 (d, J = 2.7 Hz, 1H), 8.62 (dd, J = 4.8, 1.5 Hz, 1H), 8.18 (s, 1H), 8.00 (ddd, J = 8.4, 2.7, 1.5 Hz, 1H), |
| | 7.43 (dd, J = 8.4, 4.8 Hz, 1H), 5.52 (d, J = 10.5 Hz, 1H), 4.57 (d, J = 10.5 Hz, 1H), 4.12 (q, J = 7.2 Hz, 1H), |
| | 3.49 (s, 3H), 3.34-3.20 (m, 1H), 3.19-3.07 (m, 1H), 1.67 (d, J = 7.2 Hz, 3H), 1.38 (t, J = 7.5 Hz, 3H) |
| 1-313; | 8.94 (d, J = 2.4 Hz, 1H), 8.64 (dd, J = 4.8, 1.5 Hz, 1H), 8.03 (s, 1H), 8.02 (ddd, J = 8.4, 2.4, 1.5 Hz, 1H), |
| | 7.45 (dd, J = 8.4, 4.8 Hz, 1H), 5.20-5.10 (m, 1H), 3.77 (d, J = 4.8 Hz, 2H), 3.43 (s, 3H), 3.14 (s, 3H) |
| 1-314; | (the isomer contained in a ratio of 52)8.98 (d, J = 2.7 Hz, 1H), 8.61 (dd, J = 4.8, 1.2 Hz, 1H), |
| | 8.25 (s, 1H), 8.05-7.95 (m, 1H), 7.50-7.35 (m, 1H), 3.84 (dd, J = 11.3, 3.3 Hz, 1H), 3.32 (s, 3H), |
| | 3.00-2.50 (m, 2H), 2.15-1.85 (m, 2H), 1.28 (t, J = 7.1 Hz, 3H), 1.10-0.95 (m, 3H) |
| | (the isomer contained in a ratio of 48)8.97 (d, J = 2.7 Hz, 1H), 8.60 (dd, J = 4.8, 1.2 Hz, 1H), |
| | 8.30 (s, 1H), 8.05-7.95 (m, 1H), 7.50-7.35 (m, 1H), 4.00-3.95 (m, 1H), 3.33 (s, 3H), 3.00-2.50 (m, 2H), |
| | 2.15-1.85 (m, 2H), 1.36 (t, J = 7.1 Hz, 3H), 1.10-0.95 (m, 3H) |
| 1-315; | 8.93 (d, J = 2.7 Hz, 1H), 8.63 (dd, J = 4.8, 1.2 Hz, 1H), 8.04 (s, 1H), 8.05-8.00 (m, 1H), 7.46 (dd, J = 8.3, |
| | 4.8 Hz, 1H), 4.00-3.40 (m, 3H), 1.49 (d, J = 6.5 Hz, 3H), 1.20-1.10 (m, 12H) |
| 1-318; | 8.96 (d, J = 2.7 Hz, 1H), 8.64 (dd, J = 4.8, 1.2 Hz, 1H), 8.17 (s, 1H), 8.04 (ddd, J = 8.4, 2.7, 1.2 Hz, 1H), |
| | 7.47 (dd, J = 8.4, 4.8 Hz, 1H), 4.60-4.43 (m, 1H), 4.15-4.00 (m, 1H), 3.70-3.55 (m, 1H), 2.60 (q, J = 6.9 Hz, |
| | 2H), 1.63 (d, J = 6.9 Hz, 3H), 1.29-1.23 (m, 3H), 1.11 (t, J = 6.9 Hz, 3H) |
| 1-319; | 8.93 (d, J = 2.7 Hz, 1H), 8.60 (dd, J = 4.8, 1.2 Hz, 1H), 8.17 (s, 1H), 8.02 (ddd, J = 8.4, 2.7, 1.2 Hz, 1H), |
| | 7.43 (dd, J = 8.4, 4.8 Hz, 1H), 4.70-3.10 (m, 1H), 2.71-2.59 (m, 1H), 2.56-2.44 (m, 1H), 1.57 (d, J = 6.6 Hz, |
| | 3H), 1.23 (t, J = 7.5 Hz, 3H) |
| 1-320; | 8.91 (d, J = 3.0 Hz, 1H), 8.61 (dd, J = 4.8, 1.5 Hz, 1H), 8.46 (d, J = 6.0 Hz, 2H), 8.00 (ddd, J = 8.3, 3.0, 1.5 Hz, |
| | 1H), 7.89 (s, 1H), 7.45 (dd, J = 8.3, 4.8 Hz, 1H), 7.20 (d, J = 6.0 Hz, 2H), 3.80-3.50 (m, 2H), |
| | 3.78 (d, J = 13.7 Hz, 1H), 3.74 (d, J = 13.7 Hz, 1H), 3.33 (q, J = 6.8 Hz, 1H), 1.47 (d, J = 6.8 Hz, 3H), 1.14 (t, J = 7.1 Hz, |
| | 3H) |
| 1-324; | 8.92 (d, J = 2.4 Hz, 1H), 8.63-8.55 (m, 1H), 8.09-7.90 (m, 2H), 7.42 (dd, J = 8.1, 4.8 Hz, 1H), 4.16 (q, J = 8.4 Hz, |
| | 2H), 1.43 (brs, 9H) |
| 1-328; | 8.89 (d, J = 2.4 Hz, 1H), 8.58 (dd, J = 4.8, 1.5 Hz, 1H), 8.00 (s, 1H), 8.00-7.90 (m, 1H), 7.68 (d, J = 7.2 Hz, |
| | 1H), 7.56 (t, J = 7.2H, 1H), 7.45-7.35 (m, 2H), 3.43 (s, 3H) |
| 1-331; | 8.05-8.00 (m, 2H), 7.91 (s, 1H), 7.60-7.45 (m, 2H), 7.30-7.20 (m, 3H) 3.53 (s, 2H), 3.26 (s, 3H) |
| 1-333; | 8.93 (d, J = 2.7 Hz, 1H), 8.61 (dd, J = 4.8, 1.2 Hz, 1H), 8.01 (ddd, J = 8.4, 2.7, 1.2 Hz, 1H), 7.97 (s, 1H), |
| | 7.44 (dd, J = 8.4, 4.8 Hz, 1H), 6.64 (d, J = 3.6 Hz, 1H), 6.30 (d, J = 3.6 Hz, 1H), 3.40 (s, 3H) |
| 1-335; | 8.86 (d, J = 2.7 Hz, 1H), 8.60 (dd, J = 4.8, 1.2 Hz, 1H), 7.96 (ddd, J = 8.4, 2.7, 1.2 Hz, 1H), 7.86 (s, 1H), |
| | 7.43 (dd, J = 8.4, 4.8 Hz, 1H), 3.79 (s, 2H), 3.40 (s, 3H), 2.57 (s, 3H), 2.01 (s, 3H) |
| 1-336; | 8.85-8.80 (m, 1H), 8.80-8.75 (m, 1H), 8.58 (dd, J = 4.8, 1.5 Hz, 1H), 8.30 (s, 1H), 8.05-7.95 (m, 1H), |
| | 7.95-7.85 (m, 1H), 7.40 (dd, J = 8.1, 4.8 Hz, 1H), 3.43 (s, 3H), 2.59 (s, 3H) |
| 1-337; | 8.88 (d, J = 2.7 Hz, 1H), 8.63 (d, J = 2.7 Hz, 1H), 8.49 (d, J = 2.7 Hz, 1H), 8.30-8.25 (m, 1H), 7.98 (ddd, |
| | J = 8.4, 2.7, 1.5 Hz, 1H), 7.78 (s, 1H), 7.65-7.50 (m, 1H), 7.45 (dd, J = 8.4, 4.8 Hz, 1H), 7.30-7.25 (m, |
| | 1H), 3.56 (s, 2H), 3.25 (s, 3H) |
| 1-340; | 8.96 (d, J = 2.4 Hz, 1H), 8.62 (dd, J = 4.8, 1.2 Hz, 1H), 8.10 (s, 1H), 8.04 (ddd, J = 8.1, 2.4, 1.2 Hz, 1H), |
| | 7.45 (dd, J = 8.1, 4.8 Hz, 1H), 3.85-7.70 (m, 2H), 3.59 (t, J = 6.6 Hz, 2H), 3.33 (q, J = 6.9 Hz, 1H), |
| | 2.73 (t, J = 6.6 Hz, 2H), 1.98 (qui, J = 6.6 Hz, 2H), 1.47 (d, J = 6.9 Hz, 3H), 1.17 (t, J = 7.2 Hz, 3H) |
| 1-341; | 8.94 (d, J = 2.4 Hz, 1H), 8.63-8.56 (m, 1H), 8.10 (s, 1H), 8.03 (ddd, J = 8.1, 2.4, 1.2 Hz, 1H), |
| | 7.48-7.41 (m, 1H), 5.08-4.52 (m, 1H), 3.99-3.35 (m, 1H), 2.85-2.65 (m, 2H), 2.52-2.39 (m, 1H), 1.98 (s, 3H), |
| | 1.14 (d, J = 6.6 Hz, 3H) |
| 1-342; | 8.95 (d, J = 2.4 Hz, 1H), 8.63 (dd, J = 4.8, 1.2 Hz, 1H), 8.21 (brs, 1H), 8.01 (ddd, J = 8.1, 2.4, 1.2 Hz, 1H), |
| | 7.45 (dd, J = 8.1, 4.8 Hz, 1H), 5.08-4.76 (m, 1H), 3.72-3.49 (m, 1H), 3.41-3.20 (m, 1H), |
| | 2.68-2.44 (m, 2H), 1.46 (d, J = 6.9 Hz, 3H), 1.17 (t, J = 7.8 Hz, 3H) |
| 1-343; | 8.91 (d, J = 2.4 Hz, 1H), 8.56 (dd, J = 4.8, 1.2 Hz, 1H), 8.06-7.98 (m, 1H), 7.91 (brs, 1H), 7.41 (dd, J = 8.1, |
| | 4.8 Hz, 1H), 3.42 (d, J = 7.2 Hz, 2H), 1.44 (brs, 9H), 1.08-0.94 (m, 1H), 0.54-0.44 (m, 2H), |
| | 0.22-0.11 (m, 2H) |
| 1-344; | 8.93 (d, J = 2.4 Hz, 1H), 8.62 (dd, J = 4.8, 1.5 Hz, 1H), 8.03 (ddd, J = 8.4, 2.4, 1.5 Hz, 1H), 7.98 (s, 1H), |
| | 7.45 (dd, J = 8.4, 4.8 Hz, 1H), 7.24 (d, J = 4.2 Hz, 1H), 7.02 (d, J = 4.2 Hz, 1H), 3.39 (s, 3H), 2.45 (s, 3H) |
| 1-346; | 9.25-9.15 (m, 1H), 9.15-9.10 (m, 1H), 8.85-8.75 (m, 1H), 8.60 (dd, J = 4.8, 1.5 Hz, 1H), |
| | 7.95-7.85 (m, 1H), 7.82 (s, 1H), 7.50-7.45 (m, 1H), 7.41 (dd, J = 8.4, 4.8 Hz, 1H), 3.45 (s, 3H) |
| 1-348; | 8.83 (d, J = 2.7 Hz, 1H), 8.60 (dd, J = 4.8, 1.5 Hz, 1H), 8.00-7.90 (m, 1H), 7.76 (s, 1H), 7.42 (dd, J = 8.4, |
| | 4.8 Hz, 1H), 3.40 (s, 3H), 2.33 (s, 3H), 2.27 (s, 3H) |
| 1-349; | 8.89 (d, J = 2.7 Hz, 1H), 8.64 (dd, J = 4.8, 1.5 Hz, 1H), 8.00 (ddd, J = 8.4, 2.7, 1.5 Hz, 1H), 7.94 (s, 1H), |
| | 7.46 (dd, J = 8.4, 4.8 Hz, 1H), 3.90 (q, J = 7.2 Hz, 2H), 2.90 (s, 3H), 1.28 (t, J = 7.2 Hz, 3H) |
| 1-350; | 8.94 (d, J = 2.7 Hz, 1H), 8.62 (dd, J = 4.8, 1.5 Hz, 1H), 8.03 (ddd, J = 8.4, 2.7, 1.5 Hz, 1H), 7.93 (s, 1H), |
| | 7.46 (dd, J = 8.4, 4.8 Hz, 1H), 3.75 (s, 3H), 3.72 (q, J = 7.2 Hz, 2H), 3.32 (s, 2H), 1.18 (t, J = 7.2 Hz, 3H) |
| 1-351; | 8.95 (d, J = 2.7 Hz, 1H), 8.61 (dd, J = 4.8, 1.5 Hz, 1H), 7.99 (ddd, J = 8.4, 2.7, 1.5 Hz, 1H), 7.93 (s, 1H), |
| | 7.43 (dd, J = 8.4, 4.8 Hz, 1H), 6.05 (s, 1H), 4.55-4.45 (m, 2H), 3.66 (q, J = 7.2 Hz, 2H), 2.90-2.75 (m, 2H), |
| | 1.95-1.80 (m, 1H), 1.09 (t, J = 7.2 Hz, 3H), 1.05-1.00 (m, 2H), 0.70-0.65 (m, 2H) |
| 1-352; | 8.99 (d, J = 2.7 Hz, 1H), 8.61 (dd, J = 4.8, 1.5 Hz, 1H), 8.34 (s, 1H), 8.03 (ddd, J = 8.4, 2.7, 1.5 Hz, 1H), |
| | 7.43 (dd, J = 8.4, 4.8 Hz, 1H), 4.17-4.08 (m, 4H), 3.85-3.60 (m, 2H), 2.88 (d, J = 21.9 Hz, 2H), |
| | 1.36-1.23 (m, 6H), 1.17 (t, J = 7.2 Hz, 3H) |
| 1-353; | 10.61 (brs, 1H), 9.52 (s, 1H), 8.99 (d, J = 2.7 Hz, 1H), 8.58 (dd, J = 4.8, 1.2 Hz, 1H), 8.01 (ddd, J = 8.4, |
| | 2.7, 1.2 Hz, 1H), 7.42 (dd, J = 8.4, 4.8 Hz, 1H), 4.21 (q, J = 7.2 Hz, 1H), 2.67-2.49 (m, 2H), 1.64 (d, J = 7.2 Hz, |
| | 3H), 1.29 (t, J = 7.5 Hz, 3H) |
| 1-354; | 8.96 (d, J = 2.4 Hz, 1H), 8.62 (dd, J = 4.8, 1.2 Hz, 1H), 8.09 (s, 1H), 8.05 (ddd, J = 8.1, 2.4, 1.2 Hz, 1H), |
| | 7.46 (dd, J = 8.1, 4.8 Hz, 1H), 4.56 (q, J = 6.9 Hz, 1H), 4.10-3.95 (m, 1H), 3.90-3.70 (m, 2H), |
| | 3.65-3.50 (m, 1H), 3.35-3.10 (m, 2H), 1.53 (d, J = 6.9 Hz, 3H), 1.16 (t, J = 7.2 Hz, 3H) |
| 1-355; | 8.93 (d, J = 2.4 Hz, 1H), 8.59 (dd, J = 4.8, 1.2 Hz, 1H), 8.03 (s, 1H), 8.01 (ddd, J = 8.1, 2.4, 1.2 Hz, 1H), |
| | 7.43 (dd, J = 8.1, 4.8 Hz, 1H), 3.81 (q, J = 7.2 Hz, 2H), 3.02 (s, 2H), 1.34 (s, 6H), 1.23 (t, J = 7.2 Hz, 3H) |
| 1-356; | (the isomer contained in a ratio of 5)8.93 (d, J = 2.7 Hz, 1H), 8.52 (dd, J = 5.1, 1.2 Hz, 1H), |
| | 8.51 (s, 1H), 8.00 (ddd, J = 8.4, 2.7, 1.2 Hz, 1H), 7.39 (dd, J = 8.4, 5.1 Hz, 1H), 6.92 (brs, 1H), 4.64 (q, J = 7.2 Hz, |
| | 1H), 3.88 (s, 3H), 2.65-2.44 (m, 2H), 1.45 (d, J = 7.2 Hz, 3H), 1.26 (t, J = 7.5 Hz, 3H) |
| | (the isomer contained in a ratio of 2)8.92 (d, J = 2.7 Hz, 1H), 8.58 (dd, J = 4.5, 1.2 Hz, 1H), |
| | 8.08 (s, 1H), 8.00 (ddd, J = 8.4, 2.7, 1.2 Hz, 1H), 7.57 (dd, J = 8.4, 4.5 Hz, 1H), 6.38 (brs, 1H), 3.43 (q, J = 7.2 Hz, |
| | 1H), 3.87 (s, 3H), 2.65-2.44 (m, 2H), 1.47 (d, J = 7.2 Hz, 3H), 1.24 (t, J = 7.5 Hz, 3H) |
| 1-359; | 8.96 (d, J = 2.7 Hz, 1H), 8.62 (dd, J = 4.8, 1.5 Hz, 1H), 8.05 (ddd, J = 8.3, 2.7, 1.5 Hz, 1H), 8.02 (s, 1H), |
| | 7.45 (dd, J = 8.3, 4.8 Hz, 1H), 3.76 (q, J = 7.2 Hz, 2H), 2.89 (s, 1H), 1.20 (t, J = 7.2 Hz, 3H) |
| 1-360; | 8.96 (d, J = 2.7 Hz, 1H), 8.56 (dd, J = 4.8, 1.5 Hz, 1H), 8.03 (ddd, J = 8.4, 2.7, 1.5 Hz, 1H), 8.01 (s, 1H), |
| | 7.42 (dd, J = 8.4, 4.8 Hz, 1H), 4.00-3.70 (m, 1H), 3.70-3.45 (m, 1H), 3.40-3.25 (m, 1H), 2.58 (q, J = 7.2 Hz, |
| | 2H), 2.28 (s, 3H), 1.44 (d, J = 6.9 Hz, 3H), 1.16 (t, J = 7.8 Hz, 3H), 1.15 (t, J = 7.2 Hz, 3H) |
| 1-361; | 8.83 (d, J = 2.4 Hz, 1H), 8.59 (dd, J = 4.8, 1.5 Hz, 1H), 7.93 (ddd, J = 8.4, 2.4, 1.5 Hz, 1H), 7.89 (s, 1H), |
| | 7.41 (dd, J = 8.4, 4.8 Hz, 1H), 3.93 (q, J = 7.2 Hz, 2H), 1.30 (t, J = 7.2 Hz, 3H) |
| 1-362; | 8.93 (d, J = 2.7 Hz, 1H), 8.63 (dd, J = 4.8, 1.5 Hz, 1H), 8.05 (ddd, J = 8.4, 2.7, 1.5 Hz, 1H), 7.98 (s, 1H), |
| | 7.48 (d, J = 3.9 Hz, 1H), 7.46 (dd, J = 8.4, 4.8 Hz, 1H), 7.40 (d, J = 3.9 Hz, 1H), 3.87 (q, J = 7.2 Hz, 2H), |
| | 2.49 (s, 3H), 1.28 (t, J = 7.2 Hz, 3H) |
| 1-363; | 8.96 (d, J = 2.7 Hz, 1H), 8.61 (dd, J = 4.8, 1.2 Hz, 1H), 7.99 (ddd, J = 8.4, 2.7, 1.2 Hz, 1H), 7.94 (s, 1H), |
| | 7.44 (dd, J = 8.4, 4.8 Hz, 1H), 6.24 (s, 1H), 4.35-4.30 (m, 2H), 3.64 (q, J = 7.2 Hz, 2H), 2.85-2.75 (m, 2H), |
| | 2.36 (s, 3H), 1.07 (t, J = 7.2 Hz, 3H) |
| 1-367; | (the isomer contained in a ratio of 65)8.98-8.92 (m, 1H), 8.62 (dd, J = 4.8, 1.2 Hz, 1H), |
| | 8.08-8.01 (m, 1H), 7.97 (s, 1H), 7.45 (dd, J = 8.1, 4.8 Hz, 1H), 3.78 (s, 3H), 3.70 (q, J = 7.2 Hz, 2H), 3.35 (s, |
| | 2H), 3.28 (s, 2H), 1.98 (s, 3H), 1.15 (t, J = 7.2 Hz, 3H) |
| | (the isomer contained in a ratio of 35)8.98-8.92 (m, 1H), 8.62 (dd, J = 4.8, 1.2 Hz, 1H), |
| | 8.08-8.01 (m, 1H), 7.98 (s, 1H), 7.45 (dd, J = 8.1, 4.8 Hz, 1H), 3.78 (s, 3H), 3.70 (q, J = 7.2 Hz, 2H), 3.47 (s, |
| | 2H), 3.24 (s, 2H), 1.96 (s, 3H), 1.15 (t, J = 7.2 Hz, 3H) |
| 1-370; | 9.05-8.90 (m, 1H), 8.58 (dd, J = 4.8, 1.5 Hz, 1H), 8.14 (s, 1H), 8.10-7.95 (m, 1H), 7.41 (dd, J = 8.1, 4.8 Hz, |
| | 1H), 4.20-4.00 (m, 2H), 3.50-3.30 (m, 1H), 3.10-3.00 (m, 1H), 2.5-2.35 (m, 1H), 2.25 (s, 3H), |
| | 1.59 (d, J = 7.2 Hz, 3H), 1.38 (t, J = 7.2 Hz, 3H), 1.19 (t, J = 7.2 Hz, 3H) |
| 1-371; | 8.94 (d, J = 2.4 Hz, 1H), 8.62 (dd, J = 4.8, 1.2 Hz, 1H), 8.09-8.00 (m, 2H), 7.45 (dd, J = 8.1, 4.8 Hz, 1H), |
| | 3.93-3.20 (m, 2H), 2.86 (dd, J = 12.6, 8.4 Hz, 1H), 2.78-2.64 (m, 1H), 2.47 (dd, J = 12.6, 5.4 Hz, 1H), |
| | 2.01 (s, 3H), 1.16 (d, J = 6.6 Hz, 3H), 1.04-0.90 (m, 1H), 0.54-0.43 (m, 2H), 0.24-0.15 (m, 2H) |
| 1-373; | (the isomer contained in a ratio of 4)9.00-8.90 (m, 1H), 8.65-8.60 (m, 1H), 8.05-8.00 (m, 1H), |
| | 7.94 (s, 1H), 7.50-7.40 (m, 1H), 7.24 (d, J = 4.2 Hz, 1H), 7.00 (d, J = 4.2 Hz, 1H), 3.88 (s, 3H), |
| | 3.87 (q, J = 7.2 Hz, 2H), 2.12 (s, 3H), 1.24 (t, J = 7.2 Hz, 3H) |
| | (the isomer contained in a ratio of 3)9.00-8.90 (m, 1H), 8.65-8.60 (m, 1H), 8.05-8.00 (m, 1H), |
| | 7.98 (s, 1H), 7.50-7.40 (m, 1H), 7.38 (d, J = 4.2 Hz, 1H), 7.19 (d, J = 4.2 Hz, 1H), 3.87 (q, J = 7.2 Hz, |
| | 2H), 3.80 (s, 3H), 2.24 (s, 3H), 1.26 (t, J = 7.2 Hz, 3H) |
| 1-376; | 8.95 (d, J = 2.7 Hz, 1H), |
| | 8.63 (dd, J = 4.8, 1.2 Hz, 1H), 8.04 (ddd, J = 8.3, 2.7, 1.2 Hz, 1H), 8.03 (s, 1H), 7.46 (dd, J = 8.3, 4.8 Hz, |
| | 1H), 7.37 (brs, 1H), 3.90 (s, 2H), 3.88 (s, 2H), 3.74 (q, J = 7.1 Hz, 2H), 3.44 (s, 3H), 1.19 (t, J = 7.1 Hz, |
| | 3H) |
| 1-377; | 8.84 (d, J = 2.7 Hz, 1H), 8.75-8.65 (m, 1H), 8.60 (dd, J = 4.8, 2.1 Hz, 1H), 8.00-7.95 (m, 1H), 7.92 (dd, |
| | J = 8.4, 2.1 Hz, 1H), 7.86 (s, 1H), 7.75-7.60 (m, 1H), 7.43 (dd, J = 8.4, 4.8 Hz, 1H), 3.90 (q, J = 7.2 Hz, 2H), |
| | 1.29 (t, J = 7.2 Hz, 3H) |
| 1-378; | 8.85 (d, J = 2.7 Hz, 1H), 8.59 (dd, J = 4.8, 1.5 Hz, 1H), 7.94 (s, 1H), 7.93 (ddd, J = 8.4, 2.7, 1.5 Hz, 1H), |
| | 7.86 (dd, J = 4.8, 1.2 Hz, 1H), 7.75 (dd, J = 4.2, 1.2 Hz, 1H), 7.41 (dd, J = 8.4, 4.8 Hz, 1H), 7.16 (dd, J = 4.8, |
| | 4.2 Hz, 1H), 3.90 (q, J = 7.2 Hz, 2H), 1.26 (t, J = 7.2 Hz, 3H) |
| 1-380; | 8.95 (d, J = 2.1 Hz, 1H), 8.64 (dd, J = 4.5, 1.5 Hz, 1H), 8.04 (ddd, J = 8.4, 2.1, 1.5 Hz, 1H), 7.94 (s, 1H), |
| | 7.47 (dd, J = 8.4, 4.5 Hz, 1H), 4.00-3.75 (m, 4H), 3.75-3.65 (m, 2H), 3.05-2.95 (m, 1H), |
| | 2.30-2.15 (m, 1H), 2.00-1.90 (m, 1H), 1.16 (t, J = 7.2 Hz, 3H) |
| 1-383; | 8.95 (d, J = 2.7 Hz, 1H), 8.62 (dd, J = 4.8, 1.5 Hz, 1H), 8.10 (brs, 1H), 8.04 (ddd, J = 8.4, 2.7, 1.5 Hz, 1H), |
| | 7.45 (dd, J = 8.4, 4.8 Hz, 1H), 5.52-5.41 (m, 1H), 4.90-4.40 (m, 1H), 3.75-3.61 (m, 2H), |
| | 3.42-3.30 (m, 1H), 2.66-2.52 (m, 2H), 1.49 (d, J = 6.9 Hz, 3H), 1.25 (t, J = 6.9 Hz, 3H), 1.16 (t, J = 7.5 Hz, 3H) |
| 1-386; | 8.94 (d, J = 2.7 Hz, 1H), 8.62 (dd, J = 4.8, 1.5 Hz, 1H), 8.14 (brs, 1H), 8.03 (ddd, J = 8.1, 2.7, 1.5 Hz, 1H), |
| | 7.45 (dd, J = 8.1, 4.8 Hz, 1H), 4.16-4.09 (m, 1H), 3.65-3.50 (m, 3H), 3.40-3.30 (m, 1H), 3.35 (s, 3H), |
| | 2.65-2.51 (m, 2H), 1.47 (d, J = 6.6 Hz, 3H), 1.17 (t, J = 7.5 Hz, 3H) |
| 1-387; | 8.97 (d, J = 2.4 Hz, 1H), 8.63 (dd, J = 4.8, 1.5 Hz, 1H), 8.21 (s, 1H), 8.00 (ddd, J = 8.1, 2.4, 1.5 Hz, 1H), |
| | 7.45 (dd, J = 8.1, 4.8 Hz, 1H), 4.20-4.13 (m, 1H), 4.07 (q, J = 7.2 Hz, 1H), 3.65-4.56 (m, 3H), |
| | 3.40-3.28 (m, 1H), 3.34 (s, 3H), 3.18-3.08 (m, 1H), 1.65 (d, J = 7.2 Hz, 3H), 1.38 (t, J = 7.5 Hz, 3H) |
| 1-388; | 8.96 (d, J = 2.4 Hz, 1H), 8.85 (dd, J = 4.8, 1.2 Hz, 1H), 7.96 (ddd, J = 8.1, 2.4, 1.2 Hz, 1H), 7.70 (s, 1H), |
| | 7.44 (dd, J = 8.1, 4.8 Hz, 1H), 7.30-7.05 (m, 5H), 3.90-3.65 (m, 3H), 3.65-3.40 (m, 1H), 3.30 (q, J = 6.9 Hz, |
| | 1H), 1.48 (d, J = 6.9 Hz, 3H), 1.14 (t, J = 7.2 Hz, 3H) |
| 1-390; | 8.96 (d, J = 2.4 Hz, 1H), 8.62 (dd, J = 4.8, 1.2 Hz, 1H), 8.06 (ddd, J = 8.1, 2.4, 1.2 Hz, 1H), 7.99 (s, 1H), |
| | 7.47 (dd, J = 8.1, 4.8 Hz, 1H), 3.85-3.60 (m, 2H), 3.40-3.20 (m, 1H), 2.80-2.60 (m, 1H), |
| | 2.60-2.45 (m, 1H), 2.40-2.15 (m, 2H), 2.10-2.00 (m, 1H), 2.00-1.70 (m, 2H), 1.60-1.25 (m, 3H), 1.16 (t, J = 7.2 Hz, |
| | 3H) |
| 1-395; | 8.89 (d, J = 2.7 Hz, 1H), 8.50-8.40 (m, 1H), 7.91 (ddd, J = 8.4, 2.7, 1.2 Hz, 1H), 7.81 (brs, 1H), 7.34 (dd, |
| | J = 8.4, 4.8 Hz, 1H), 4.02 (s, 3H), 3.59 (q, J = 7.2 Hz, 2H), 1.47 (s, 9H), 1.15 (t, J = 7.2 Hz, 3H) |
| 1-397; | 8.66 (d, J = 2.4 Hz, 1H), 8.56 (dd, J = 4.8, 1.2 Hz, 1H), 8.26 (d, J = 2.1 Hz, 1H), 7.88 (s, 1H), 7.85 (ddd, J = 8.1, |
| | 2.4, 1.2 Hz, 1H), 7.55 (dd, J = 5.4, 2.1 Hz, 1H), 7.37 (dd, J = 8.1, 4.8 Hz, 1H), 7.18 (d, J = 5.4 Hz, 1H), |
| | 4.91 (q, J = 6.9 Hz, 1H), 3.90-3.60 (m, 2H), 1.56 (d, J = 6.9 Hz, 3H), 1.17 (t, J = 7.2 Hz, 3H) |
| 1-399; | (the isomer contained in a ratio of 9)8.92 (d, J = 2.7 Hz, 1H), 8.63 (dd, J = 5.1, 1.5 Hz, 1H), |
| | 8.03 (ddd, J = 8.1, 2.7, 1.5 Hz, 1H), 7.99 (s, 1H), 7.46 (dd, J = 8.1, 5.1 Hz, 1H), 4.95-4.20 (m, 2H), |
| | 3.81 (s, 3H), 2.88 (dd, J = 12.3, 8.7 Hz, 1H), 2.80-2.75 (m, 1H), 2.48 (dd, J = 12.3, 5.1 Hz, 1H), |
| | 2.03-2.00 (m, 3H), 1.92 (s, 3H), 1.19 (d, J = 6.6 Hz, 3H) |
| | (the isomer contained in a ratio of 1)8.92 (d, J = 2.7 Hz, 1H), 8.63 (dd, J = 5.1, 1.5 Hz, 1H), |
| | 8.03 (ddd, J = 8.1, 2.7, 1.5 Hz, 1H), 7.99 (s, 1H), 7.46 (dd, J = 8.1, 5.1 Hz, 1H), 4.95-4.20 (m, 2H), |
| | 3.72 (s, 3H), 2.88 (dd, J = 12.3, 8.7 Hz, 1H), 2.80-2.75 (m, 1H), 2.48 (dd, J = 12.3, 5.1 Hz, 1H), |
| | 2.03-2.00 (m, 3H), 1.89 (s, 3H), 1.18 (d, J = 6.6 Hz, 3H) |
| 1-400; | 8.92 (d, J = 2.4 Hz, 1H), 8.63-8.57 (m, 1H), 8.02 (ddd, J = 8.1, 2.4, 1.2 Hz, 1H), 7.96 (brs, 1H), 7.44 (dd, |
| | J = 8.1, 4.8 Hz, 1H), 3.40-3.00 (m, 2H), 2.84 (dd, J = 12.3, 9.0 Hz, 1H), 2.82-2.60 (m, 1H)2.44 (dd, |
| | J = 12.3, 4.2 Hz, 1H), 1.99 (s, 3H), 1.13 (d, J = 6.6 Hz, 3H), 0.07 (s, 9H) |
| 1-402; | 8.97 (d, J = 2.7 Hz, 1H), 8.63 (dd, J = 4.8, 1.5 Hz, 1H), 7.96 (s, 1H), 8.07 (ddd, J = 8.4, 2.7, 1.5 Hz, 1H), |
| | 7.47 (dd, J = 8.4, 4.8 Hz, 1H), 4.15-4.00 (m, 2H), 3.75-3.60 (m, 2H), 2.65-2.50 (m, 2H), |
| | 2.45-2.30 (m, 1H), 1.80-1.50 (m, 4H), 1.44 (s, 9H), 1.15 (t, J = 7.2 Hz, 3H) |
| 1-403; | 9.10-9.00 (m, 1H), 8.62 (dd, J = 4.8, 1.5 Hz, 1H), 8.20-8.05 (m, 2H), 7.45-7.40 (m, 1H), |
| | 4.25-4.05 (m, 1H), 4.05-3.85 (m, 1H), 2.85-2.60 (m, 2H), 2.40-2.30 (m, 1H), 1.85-1.75 (m, 2H), 1.70-1.50 (m, |
| | 2H), 1.36 (s, 9H), 1.35-1.20 (m, 2H), 1.13 (t, J = 7.2 Hz, 3H) |
| 1-404; | 8.90-8.80 (m, 1H), 8.60 (dd, J = 4.8, 1.5 Hz, 1H), 7.95-7.85 (m, 1H), 7.84 (s, 1H), 7.41 (dd, J = 8.4, 4.8 Hz, |
| | 1H), 4.61 (s, 2H), 3.86 (q, J = 7.2 Hz, 2H), 3.45 (s, 3H), 1.27 (t, J = 7.2 Hz, 3H) |
| 1-405; | 8.85 (d, J = 2.4 Hz, 1H), 8.57 (dd, J = 4.8, 1.5 Hz, 1H), 7.95-7.90 (m, 1H), 7.86 (s, 1H), 7.38 (dd, J = 8.4, |
| | 4.8 Hz, 1H), 5.82 (brs, 2H), 4.04 (s, 3H), 3.10 (q, J = 7.2 Hz, 2H), 1.25 (t, J = 7.2 Hz, 3H) |
| 1-406; | 8.90 (d, J = 2.4 Hz, 1H), 8.58 (dd, J = 4.8, 1.5 Hz, 1H), 8.05-7.95 (m, 1H), 7.75 (s, 1H), 7.42 (dd, J = 8.1, |
| | 4.8 Hz, 1H), 7.04 (dd, J = 4.2, 1.2 Hz, 1H), 6.13 (dd, J = 3.9, 1.2 Hz, 1H), 5.88 (dd, J = 4.2, 3.9 Hz, 1H), |
| | 3.88 (s, 3H), 3.83 (q, J = 7.2 Hz, 2H), 1.26 (t, J = 7.2 Hz, 3H) |
| 1-407; | 8.96 (d, J = 2.7 Hz, 1H), 8.64 (dd, J = 4.8, 1.2 Hz, 1H), 8.05 (ddd, J = 8.3, 2.7, 1.2 Hz, 1H), 8.03 (s, 1H), |
| | 7.47 (dd, J = 8.3, 4.8 Hz, 1H), 3.73 (q, J = 7.1 Hz, 2H), 3.63 (d, J = 4.5 Hz, 2H), 2.77 (s, 6H), 1.18 (t, J = 7.1 Hz, |
| | 3H) |
| 1-409; | 8.95 (d, J = 3.0 Hz, 1H), 8.66 (dd, J = 4.8, 1.2 Hz, 1H), 8.06 (ddd, J = 8.3, 3.0, 1.2 Hz, 1H), 8.01 (s, 1H), |
| | 7.48 (dd, J = 8.3, 4.8 Hz, 1H), 7.40-7.35 (m, 1H), 3.92 (d, J = 3.9 Hz, 2H), 3.76 (q, J = 7.1 Hz, 2H), |
| | 1.21 (t, J = 7.1 Hz, 3H) |
| 1-411; | 8.95 (d, J = 2.7 Hz, 1H), 8.62 (dd, J = 4.8, 1.2 Hz, 1H), 8.10-8.00 (m, 1H), 7.98 (s, 1H), 7.46 (dd, J = 8.3, |
| | 4.8 Hz, 1H), 3.71 (q, J = 7.1 Hz, 2H), 2.93 (s, 2H), 2.23 (s, 6H), 1.16 (t, J = 7.1 Hz, 3H) |
| 1-417; | 8.95 (d, J = 2.7 Hz, 1H), 8.62 (dd, J = 4.8, 1.2 Hz, 1H), 8.05-7.90 (m, 1H), 8.03 (s, 1H), 7.46 (dd, J = 8.3, |
| | 4.8 Hz, 1H), 3.84 (s, 2H), 3.76 (q, J = 7.1 Hz, 2H), 2.92 (s, 3H), 1.43 (s, 9H), 1.16 (t, J = 7.1 Hz, 3H) |
| 1-418; | 8.95 (d, J = 2.4 Hz, 1H), 8.62 (dd, J = 4.8, 1.5 Hz, 1H), 8.04 (ddd, J = 8.4, 2.4, 1.5 Hz, 1H), 7.93 (s, 1H), |
| | 7.46 (dd, J = 8.4, 4.8 Hz, 1H), 3.71 (q, J = 7.2 Hz, 2H), 2.75-2.55 (m, 2H), 2.38 (t, J = 7.5 Hz, 2H), |
| | 1.17 (t, J = 7.2 Hz, 3H) |
| 1-420; | 8.94 (d, J = 2.7 Hz, 1H), 8.62 (dd, J = 4.8, 1.5 Hz, 1H), 8.03 (ddd, J = 8.4, 2.7, 1.5 Hz, 1H), 7.94 (s, 1H), |
| | 7.45 (dd, J = 8.4, 4.8 Hz, 1H), 5.25-5.15 (m, 1H), 3.70 (q, J = 7.2 Hz, 2H), 3.37 (q, J = 6.0 Hz, 2H), |
| | 2.33 (t, J = 6.0 Hz, 2H), 1.55 (s, 9H), 1.16 (t, J = 7.2 Hz, 3H) |
| 1-421; | 8.97 (d, J = 2.7 Hz, 1H), 8.70-8.60 (m, 2H), 8.55-8.50 (m, 1H), 8.10-8.05 (m, 1H), 7.99 (s, 1H), |
| | 7.73 (d, J = 15.3 Hz, 1H), 7.70-7.65 (m, 1H), 7.46 (dd, J = 8.4, 4.8 Hz, 1H), 7.30-7.20 (m, 1H), 6.49 (d, J = 15.3 Hz, |
| | 1H), 3.83 (q, J = 7.2 Hz, 2H), 1.24 (t, J = 7.2 Hz, 3H) |
| 1-422; | 8.98 (d, J = 2.7 Hz, 1H), 8.65-8.60 (m, 1H), 8.10-8.05 (m, 1H), 7.97 (s, 1H), 7.50 (d, J = 15.3 Hz, 1H), |
| | 7.46 (dd, J = 8.4, 4.8 Hz, 1H), 7.40-7.30 (m, 1H), 6.60-6.55 (m, 1H), 6.45-6.40 (m, 1H), 6.30 (d, J = 15.3 Hz, |
| | 1H), 3.81 (q, J = 7.2 Hz, 2H), 1.22 (t, J = 7.2 Hz, 3H) |
| 1-423; | 8.97 (d, J = 2.7 Hz, 1H), 8.62 (dd, J = 4.8, 1.5 Hz, 1H), 8.06 (ddd, J = 8.4, 2.7, 1.5 Hz, 1H), 7.97 (s, 1H), |
| | 7.85 (d, J = 15.3 Hz, 1H), 7.45 (dd, J = 8.4, 4.8 Hz, 1H), 7.30-7.25 (m, 1H), 7.20-7.15 (m, 1H), 6.99 (dd, |
| | J = 4.8, 3.6 Hz, 1H), 6.21 (d, J = 15.3 Hz, 1H), 3.82 (q, J = 7.2 Hz, 2H), 1.22 (t, J = 7.2 Hz, 3H) |
| 1-424; | 8.94 (d, J = 2.7 Hz, 1H), 8.62 (dd, J = 4.8, 1.5 Hz, 1H), 8.06 (s, 1H), 8.01 (ddd, J = 8.4, 2.7, 1.5 Hz, 1H), |
| | 7.46 (dd, J = 8.4, 4.8 Hz, 1H), 4.13 (q, J = 7.2 Hz, 2H), 3.77 (q, J = 7.2 Hz, 2H), 1.20 (t, J = 7.2 Hz, 3H), |
| | 1.13 (t, J = 7.2 Hz, 3H) |
| 1-426; | 8.97 (d, J = 2.7 Hz, 1H), 8.61 (dd, J = 4.8, 1.2 Hz, 1H), 8.06 (ddd, J = 8.4, 2.7, 1.2 Hz, 1H), 7.97 (s, 1H), |
| | 7.47 (dd, J = 8.4, 4.8 Hz, 1H), 4.10-3.40 (m, 2H), 1.91 (s, 3H), 1.59 (s, 6H), 1.15 (t, J = 7.2 Hz, 3H) |
| 1-427; | 8.95 (d, J = 2.4 Hz, 1H), 8.63 (dd, J = 4.5, 1.5 Hz, 1H), 8.04 (ddd, J = 8.4, 2.4, 1.5 Hz, 1H), 8.00 (s, 1H), |
| | 7.46 (dd, J = 8.4, 4.5 Hz, 1H), 4.15-3.35 (m, 2H), 1.24 (t, J = 7.2 Hz, 3H) |
| 1-429; | 8.97 (d, J = 2.4 Hz, 1H), 8.64 (dd, J = 4.8, 1.5, 1H), 8.15-8.05 (m, 1H), 7.93 (brs, 1H), 7.46 (dd, J = 8.4, |
| | 4.8 Hz, 1H), 3.60 (q, J = 7.2 Hz, 2H), 1.41 (s, 9H), 1.17 (t, J = 7.2 Hz, 3H) |
| 1-430; | 8.98 (d, J = 2.7 Hz, 1H), 8.63 (dd, J = 4.8, 1.5 Hz, 1H), 8.28 (brs, 1H), 8.05 (ddd, J = 8.1, 2.7, 1.5 Hz, 1H), |
| | 7.46 (dd, J = 8.1, 4.8 Hz, 1H), 5.70-5.20 (m, 1H), 4.30-3.80 (m, 1H), 3.40-3.29 (m, 1H), |
| | 2.67-2.50 (m, 2H), 2.24 (s, 3H), 1.48 (d, J = 6.6 Hz, 3H), 1.17 (t, J = 7.5 Hz, 3H) |
| 1-431; | 8.97 (d, J = 2.4 Hz, 1H), 8.61 (dd, J = 4.8, 1.2 Hz, 1H), 8.10-8.05 (m, 1H), 8.05 (s, 1H), 7.44 (dd, J = 8.1, |
| | 4.8 Hz, 1H), 7.00 (s, 1H), 6.96 (s, 1H), 4.17 (q, J = 6.9 Hz, 1H), 3.95-3.75 (m, 1H), 3.69 (s, 3H), |
| | 3.50-3.30 (m, 1H), 1.49 (d, J = 6.9 Hz, 3H), 1.05 (t, J = 7.2 Hz, 3H) |
| 1-432; | 8.65 (d, J = 2.4 Hz, 1H), 8.55 (dd, J = 4.8, 1.2 Hz, 1H), 8.26 (dd, J = 3.9, 1.2 Hz, 1H), 7.90 (s, 1H), |
| | 7.83 (ddd, J = 8.1, 2.4, 1.2 Hz, 1H), 7.60-7.7.55 (m, 1H), 7.40-7.30 (m, 1H), 7.10-7.05 (m, 1H), |
| | 6.80-6.70 (m, 1H), 4.82 (q, J = 6.9 Hz, 1H), 3.95-3.75 (m, 1H), 3.70-3.50 (m, 1H), 1.55 (d, J = 6.9 Hz, 3H), 1.15 (t, |
| | J = 7.2 Hz, 3H) |
| 1-433; | 8.86 (d, J = 2.4 Hz, 1H), 8.80-8.65 (m, 1H), 8.56 (dd, J = 4.8, 1.2 Hz, 1H), 8.11 (dd, J = 9.0, 2.7 Hz, 1H), |
| | 7.94 (ddd, J = 8.1, 2.4, 1.2 Hz, 1H), 7.93 (s, 1H), 7.41 (dd, J = 8.1, 4.8 Hz, 1H), 7.22 (d, J = 9.0 Hz, 1H), |
| | 4.93 (q, J = 6.9 Hz, 1H), 3.90-3.55 (m, 2H), 1.58 (d, J = 6.9 Hz, 3H), 1.17 (t, J = 7.2 Hz, 3H) |
| 1-434; | 8.95 (d, J = 2.4 Hz, 1H), 8.62 (dd, J = 4.8, 1.2 Hz, 1H), 8.07 (s, 1H), 8.03 (ddd, J = 8.1, 2.4, 1.2 Hz, 1H), |
| | 7.45 (dd, J = 8.1, 4.8 Hz, 1H), 4.49 (t, J = 5.4 Hz, 1H), 3.71 (q, J = 7.2 Hz, 2H), 3.34 (s, 6H), 3.21 (s, 2H), |
| | 2.83 (d, J = 5.4 Hz, 2H), 1.17 (t, J = 7.2 Hz, 3H) |
| 1-435; | 8.96 (brs, 1H), 8.43 (dd, J = 4.8, 1.5 Hz, 1H), 8.24 (brs, 1H), 8.00-7.90 (m, 1H), 7.31 (dd, J = 8.4, 4.8 Hz, |
| | 1H), 6.38 (brs, 1H), 2.64 (q, J = 7.5 Hz, 2H), 1.50 (s, 9H), 1.32 (t, J = 7.5 Hz, 3H) |
| 1-439; | 8.94 (d, J = 2.7 Hz, 1H), 8.65-8.60 (m, 1H), 8.05-8.00 (m, 1H), 7.95 (s, 1H), 7.45 (dd, J = 8.4, 4.8 Hz, 1H), |
| | 3.86 (s, 2H), 3.70 (q, J = 7.5 Hz, 2H), 1.17 (t, J = 7.5 Hz, 3H), 1.09 (s, 9H) |
| 1-440; | 8.96 (d, J = 2.4 Hz, 1H), 8.65-8.60 (m, 1H), 8.10-8.05 (m, 1H), 7.94 (s, 1H), 7.47 (dd, J = 8.4, 4.8 Hz, 1H), |
| | 4.00-3.85 (m, 2H), 3.75-3.60 (m, 2H), 3.30-3.20 (m, 2H), 2.55-2.45 (m, 1H), 2.00-1.85 (m, 2H), |
| | 1.60-1.50 (m, 2H), 1.15 (t, J = 7.2 Hz, 3H) |
| 1-443; | 8.96 (d, J = 2.7 Hz, 1H), 8.61 (dd, J = 4.8, 1.2 Hz, 1H), 8.10-8.05 (m, 1H), 7.99 (s, 1H), 7.45 (d, J = 8.1, |
| | 4.8 Hz, 1H), 3.80-3.60 (m, 2H), 1.45-1.35 (m, 1H), 1.25-1.20 (m, 1H), 1.20-1.10 (m, 1H), 1.15 (t, J = 7.2 Hz, |
| | 3H), 1.01 (d, J = 6.0 Hz, 3H), 0.60-0.50 (m, 1H) |
| 1-444; | 8.97 (d, J = 2.4 Hz, 1H), 8.65-8.60 (m, 1H), 8.10-8.05 (m, 1H), 7.95 (s, 1H), 7.46 (dd, J = 8.4, 4.8 Hz, 1H), |
| | 3.69 (q, J = 7.2 Hz, 2H), 2.08 (s, 2H), 1.16 (t, J = 7.2 Hz, 3H), 1.01 (s, 9H) |
| 1-445; | 8.95 (d, J = 2.4 Hz, 1H), 8.60 (dd, J = 4.8, 1.5 Hz, 1H), 8.07 (ddd, J = 8.4, 2.4, 1.5 Hz, 1H), 7.92 (s, 1H), |
| | 7.47 (dd, J = 8.4, 4.8 Hz, 1H), 5.15-5.10 (m, 2H), 3.75 (q, J = 7.2 Hz, 2H), 1.89 (s, 3H), 1.19 (t, J = 7.2 Hz, |
| | 3H) |
| 1-452; | 8.95 (d, J = 2.4 Hz, 1H), 8.62 (dd, J = 4.8, 1.2 Hz, 1H), 8.05 (ddd, J = 8.1, 2.4, 1.2 Hz, 1H), 7.99 (s, 1H), |
| | 7.46 (dd, J = 8.1, 4.8 Hz, 1H), 4.58 (t, J = 7.2 Hz, 1H), 3.73 (q, J = 7.2 Hz, 2H), 3.10-2.75 (m, 4H), |
| | 2.56 (d, J = 7.2 Hz, 2H), 2.20-2.00 (m, 1H), 1.90-1.70 (m, 1H), 1.17 (t, J = 7.2 Hz, 3H) |
| 1-453; | 8.97 (d, J = 2.4 Hz, 1H), 8.59 (dd, J = 4.8, 1.5 Hz, 1H), 8.10 (s, 1H), 8.05 (ddd, J = 8.1, 2.4, 1.5 Hz, 1H), |
| | 7.44 (dd, J = 8.1, 4.8 Hz, 1H), 4.51 (d, J = 11.4 Hz, 1H), 4.44 (d, J = 11.4 Hz, 1H), 3.60-3.40 (m, 1H), |
| | 3.43 (s, 3H), 3.26 (s, 3H), 2.70-2.50 (m, 2H), 1.44 (d, J = 6.9 Hz, 3H), 1.17 (t, J = 7.2 Hz, 3H) |
| 1-456; | 8.95 (d, J = 2.4 Hz, 1H), 8.62 (dd, J = 4.8, 1.2 Hz, 1H), 8.06 (ddd, J = 8.1, 2.4, 1.2 Hz, 1H), 7.92 (s, 1H), |
| | 7.46 (dd, J = 8.1, 4.8 Hz, 1H), 3.70 (q, J = 7.2 Hz, 2H), 2.13 (t, J = 7.2 Hz, 2H), 1.65-1.50 (m, 4H), |
| | 1.30-1.20 (m, 9H), 1.15 (t, J = 7.2 Hz, 2H), 0.88-0.82 (m, 3H) |
| 1-457; | 8.95 (d, J = 2.7 Hz, 1H), 8.61 (dd, J = 4.8, 1.5 Hz, 1H), 8.05 (ddd, J = 8.4, 2.7, 1.5 Hz, 1H), 7.91 (s, 1H), |
| | 7.45 (dd, J = 8.4, 4.8 Hz, 1H), 3.70 (q, J = 7.2 Hz, 2H), 2.13 (t, J = 7.2 Hz, 2H), 1.67-1.50 (m, 4H), |
| | 1.27-1.13 (m, 17H), 0.89-0.84 (m, 3H) |
| 1-458; | 8.93 (d, J = 2.7 Hz, 1H), 8.60 (dd, J = 4.8, 1.2 Hz, 1H), 8.04 (ddd, J = 8.4, 2.7, 1.2 Hz, 1H), 7.92 (s, 1H), |
| | 7.44 (dd, J = 8.4, 4.8 Hz, 1H), 3.62 (q, J = 7.2 Hz, 2H), 1.14 (t, J = 7.2 Hz, 3H), 1.13 (s, 3H), |
| | 1.10-1.05 (m, 2H), 0.50-0.45 (m, 2H) |
| 1-459; | 8.94 (d, J = 2.7 Hz, 1H), 8.65-8.60 (m, 1H), 8.10-8.00 (m, 1H), 7.86 (s, 1H), 7.45 (dd, J = 8.4, 4.8 Hz, 1H), |
| | 3.68 (q, J = 7.2 Hz, 2H), 3.15-3.00 (m, 1H), 2.40-2.20 (m, 2H), 1.95-1.75 (m, 4H), 1.14 (t, J = 7.2 Hz, |
| | 3H) |
| 1-461; | 8.94 (d, J = 2.4 Hz, 1H), 8.61 (dd, J = 4.8, 1.2 Hz, 1H), 8.03 (ddd, J = 8.4, 2.4, 1.2 Hz, 1H), 7.91 (s, 1H), |
| | 7.44 (dd, J = 8.4, 4.8 Hz, 1H), 3.71 (q, J = 7.2 Hz, 2H), 2.07 (d, J = 6.9 Hz, 2H), 1.16 (t, J = 7.2 Hz, 3H), |
| | 1.10-1.00 (m, 1H), 0.55-0.45 (m, 2H), 0.10-0.05 (m, 2H) |
| 1-462; | 8.95 (d, J = 2.7 Hz, 1H), 8.64 (dd, J = 4.5, 1.5 Hz, 1H), 8.05 (ddd, J = 8.4, 2.7, 1.5 Hz, 1H), 7.98 (s, 1H), |
| | 7.47 (dd, J = 8.4, 4.5 Hz, 1H), 5.82 (s, 1H), 4.40 (dd, J = 9.0, 3.3 Hz, 1H), 3.73 (q, J = 7.2 Hz, 2H), |
| | 3.04 (dd, J = 17.1, 3.3 Hz, 1H), 2.75 (dd, J = 17.1, 9.0 Hz, 1H), 1.18 (t, J = 7.2 Hz, 3H) |
| 1-463; | 8.95 (d, J = 2.4 Hz, 1H), 8.64 (dd, J = 4.8, 1.5 Hz, 1H), 8.04 (ddd, J = 8.4, 2.4, 1.5 Hz, 1H), 8.00 (s, 1H), |
| | 7.47 (dd, J = 8.4, 4.8 Hz, 1H), 5.96 (s, 1H), 4.75-4.70 (m, 1H), 3.80-3.60 (m, 2H), 2.86 (dd, J = 17.1, 4.2 Hz, |
| | 1H), 2.85 (dd, J = 17.1, 3.3 Hz, 1H), 1.18 (t, J = 7.2 Hz, 3H) |
| 1-465; | (the isomer contained in a ratio of 2)8.95 (d, J = 2.4 Hz, 1H), 8.62 (dd, J = 4.8, 1.2 Hz, 1H), |
| | 8.63 (s, 1H), 8.03 (ddd, J = 8.1, 2.4, 1.2 Hz, 1H), 7.46 (dd, J = 8.1, 4.8 Hz, 1H), 7.27 (t, J = 6.3 Hz, 1H), |
| | 3.77 (s, 3H), 3.71 (q, J = 7.2 Hz, 2H), 3.30 (d, J = 6.3H, 2H), 3.15 (s, 2H), 1.17 (t, J = 7.2 Hz, 3H) |
| | (the isomer contained in a ratio of 1)8.95 (d, J = 2.4 Hz, 1H), 8.62 (dd, J = 4.8, 1.2 Hz, 1H), |
| | 8.63 (s, 1H), 8.03 (ddd, J = 8.1, 2.4, 1.2 Hz, 1H), 7.46 (dd, J = 8.1, 4.8 Hz, 1H), 6.74 (t, J = 6.3 Hz, 1H), |
| | 3.82 (s, 3H), 3.71 (q, J = 7.2 Hz, 2H), 3.44 (d, J = 6.3H, 2H), 3.17 (s, 2H), 1.17 (t, J = 7.2 Hz, 3H) |
| 1-467; | 8.99 (d, J = 2.7 Hz, 1H), 8.51 (dd, J = 4.8, 1.5 Hz, 1H), 8.35 (s, 1H), 8.01 (ddd, J = 8.1, 2.7, 1.5 Hz, 1H), |
| | 7.37 (dd, J = 8.1, 4.8 Hz, 1H), 6.54 (brs, 1H), 2.50 (s, 3H), 1.54 (s, 9H) |
| 1-469; | 8.70 (d, J = 2.4 Hz, 1H), 8.55 (dd, J = 4.8, 1.5 Hz, 1H), 8.00-7.90 (m, 1H), 7.85-7.80 (m, 1H), 7.82 (s, 1H), |
| | 7.50-7.30 (m, 2H), 7.20-7.15 (m, 2H), 3.45 (s, 3H) |
| 1-472; | 8.65-8.75 (m, 1H), 8.55 (d, J = 3.6 Hz, 1H), 7.80-8.00 (m, 2H), 7.30-7.45 (m, 2H), 7.10-7.25 (m, 2H), |
| | 7.00-7.15 (m, 1H), 3.90 (m, 2H), 2.48 (s, 3H), 1.20-1.40 (m, 3H) |
| 1-473; | 8.77 (d, J = 2.4 Hz, 1H), 8.56 (dd, J = 4.8, 1.5 Hz, 1H), 7.85-7.95 (m, 1H), 7.66 (brs, 1H), 7.38 (dd, J = 8.1, |
| | 4.8 Hz, 1H), 7.25-7.35 (m, 1H), 7.10-7.20 (m, 3H), 3.86 (q, J = 7.2 Hz, 2H), 2.41 (s, 3H), 1.26 (t, J = 7.2 Hz, |
| | 3H) |
| 1-474; | 8.75-8.85 (m, 1H), 8.50-8.60 (m, 1H), 7.85-7.95 (m, 1H), 7.70 (s, 1H), 7.39 (dd, J = 8.7, 4.8 Hz, 1H), |
| | 7.33 (d, J = 8.7 Hz, 2H), 7.08 (d, J = 8.7 Hz, 2H), 3.85 (q, J = 7.2 Hz, 2H), 2.43 (s, 3H), 1.25 (t, J = 7.2 Hz, |
| | 3H) |
| 1-475; | 8.78 (d, J = 3.0 Hz, 1H), 8.57 (dd, J = 4.8, 1.5 Hz, 1H), 7.88 (d, J = 8.1 Hz, 1H), 7.72 (s, 1H), 7.66 (s, 1H), |
| | 7.55-7.65 (m, 2H), 7.30-7.45 (m, 2H), 3.88 (q, J = 7.2 Hz, 2H), 1.27 (t, J = 7.2 Hz, 3H) |
| 1-476; | 8.82 (d, J = 2.4 Hz, 1H), 8.58 (dd, J = 4.8, 1.5 Hz, 1H), 7.94 (ddd, J = 8.4, 2.4, 1.5 Hz, 1H), 7.75 (s, 1H), |
| | 7.40 (dd, J = 8.4, 4.8 Hz, 1H), 7.19 (s, 1H), 7.16 (d, J = 8.1 Hz, 1H), 6.93 (d, J = 8.1 Hz, 1H), 3.85 (q, J = 7.2 Hz, |
| | 2H), 1.26 (t, J = 7.2 Hz, 3H) |
| 1-477; | (the isomer contained in a ratio of 4)8.98-8.93 (m, 2H), 8.63-8.60 (m, 1H), 8.10-8.00 (m, 2H), |
| | 7.50-7.40 (m, 1H), 3.69 (s, 3H), 2.95-2.80 (m, 2H), 2.60-2.45 (m, 1H), 2.05-2.03 (m, 3H), |
| | 1.28-1.20 (m, 3H) |
| | (the isomer contained in a ratio of 1)8.98-8.93 (m, 2H), 8.63-8.60 (m, 1H), 8.10-8.00 (m, 2H), |
| | 7.50-7.40 (m, 1H), 3.68 (s, 3H), 2.95-2.80 (m, 2H), 2.60-2.45 (m, 1H), 2.05-2.03 (m, 3H), |
| | 1.28-1.20 (m, 3H) |
| 1-481; | (the isomer contained in a ratio of 11)8.95 (d, J = 2.4 Hz, 1H), 8.56 (dd, J = 4.8, 1.5 Hz, 1H), |
| | 8.05-8.01 (m, 2H), 7.70 (s, 1H), 7.42 (dd, J = 8.4, 4.8 Hz, 1H), 6.41 (brs, 1H), 3.86 (s, 3H), 3.49 (q, J = 7.2 Hz, |
| | 1H), 2.63-2.56 (m, 2H), 1.43 (d, J = 7.2 Hz, 3H), 1.22 (t, J = 7.5 Hz, 3H) |
| | (the isomer contained in a ratio of 9)8.97 (d, J = 2.7 Hz, 1H), 8.50 (dd, J = 4.8, 1.5 Hz, 1H), |
| | 8.42 (s, 1H), 8.05-8.01 (m, 1H), 7.67 (s, 1H), 7.39 (dd, J = 9.0, 4.8 Hz, 1H), 6.76 (brs, 1H), 4.67 (q, J = 7.2 Hz, |
| | 1H), 3.86 (s, 3H), 2.54-2.45 (m, 2H), 1.47 (d, J = 7.2 Hz, 3H), 1.26 (t, J = 7.2 Hz, 3H) |
| 1-484; | 8.96 (d, J = 2.7 Hz, 1H), 8.56 (dd, J = 4.8, 1.8 Hz, 1H), 8.10-8.00 (m, 1H), 7.90 (brs, 1H), 7.41 (dd, J = 8.7, |
| | 4.8 Hz, 1H), 4.30-3.90 (m, 1H), 3.40-3.10 (m, 1H), 2.90-2.30 (m, 5H), 2.00 (s, 3H), 1.40-1.20 (m, |
| | 3H), 1.20-1.00 (m, 6H) |
| 1-486; | (the isomer contained in a ratio of 7)8.92 (d, J = 2.7 Hz, 1H), 8.49 (dd, J = 4.8, 1.5 Hz, 1H), |
| | 8.02-7.98 (m, 1H), 7.55 (s, 1H), 7.41 (s, 1H), 7.38 (dd, J = 8.1, 4.8 Hz, 1H), 3.86 (s, 3H), 3.71 (q, J = 7.2 Hz, |
| | 1H), 3.24 (s, 3H), 2.62-2.52 (m, 2H), 1.52 (d, J = 7.2 Hz, 3H), 1.21 (t, J = 7.5 Hz, 3H) |
| | (the isomer contained in a ratio of 1)8.95-8.94 (m, 1H), 8.54-8.52 (m, 1H), 8.02-7.98 (m, 1H), |
| | 7.97 (s, 1H), 7.41 (s, 1H), 7.38 (dd, J = 8.1, 4.8 Hz, 1H), 3.82 (s, 3H), 3.71 (q, J = 7.2 Hz, 1H), |
| | 3.17 (s, 3H), 2.62-2.52 (m, 2H), 1.52 (d, J = 7.2 Hz, 3H), 1.21 (t, J = 7.5 Hz, 3H) |
| 1-487; | 8.97 (d, J = 2.4 Hz, 1H), 8.66 (dd, J = 4.8, 1.5 Hz, 1H), 7.91 (ddd, J = 8.4, 2.7, 1.2 Hz, 1H), 7.97 (s, 1H), |
| | 7.47 (dd, J = 8.4, 4.8 Hz, 1H), 3.64 (q, J = 7.2 Hz, 2H), 3.28 (s, 3H), 1.51 (s, 9H), 1.20 (t, J = 7.2 Hz, 3H) |
| 1-488; | 9.20-9.05 (m, 1H), 8.70-8.35 (m, 2H), 8.20-8.10 (m, 1H), 7.50-7.35 (m, 1H), 4.25-4.10 (m, 1H), |
| | 3.60-3.40 (m, 1H), 3.40-3.30 (m, 1H), 3.30-3.15 (m, 1H), 3.05-2.85 (m, 1H), 2.80-2.75 (m, 3H), |
| | 1.30-1.15 (m, 6H) |
| 1-489; | 9.05-8.95 (m, 1H), 8.60-8.50 (m, 1H), 8.15-8.05 (m, 1H), 8.05-8.00 (m, 1H), 7.45-7.35 (m, 1H), |
| | 4.15-3.80 (m, 1H), 3.55-3.30 (m, 1H), 3.20-2.65 (m, 2H), 2.60-2.55 (m, 3H), 2.55-2.50 (m, 1H), |
| | 2.35-2.30 (m, 3H), 1.25-1.15 (m, 3H), 1.15-1.10 (m, 3H) |
| 1-490; | 9.01 (d, J = 2.1 Hz, 1H), 8.56 (dd, J = 4.8, 1.2 Hz, 1H), 8.09 (s, 1H), 8.05-7.95 (m, 1H), 7.41 (dd, J = 8.4, |
| | 4.8 Hz, 1H), 4.30-3.95 (m, 1H), 3.84 (dd, J = 13.5, 10.8 Hz, 1H), 3.45-3.20 (m, 2H), 2.94 (s, 3H), |
| | 2.87 (dd, J = 13.5, 3.0 Hz, 1H), 2.32 (s, 3H), 1.16 (t, J = 6.9 Hz, 3H), 1.15 (d, J = 6.9 Hz, 3H) |
| 1-491; | 9.10-9.00 (m, 1H), 8.65-8.60 (m, 1H), 8.25-8.10 (m, 1H), 8.10-8.00 (m, 1H), 7.50-7.40 (m, 1H), |
| | 4.20-4.00 (m, 2H), 3.45-3.30 (m, 2H), 3.30-3.25 (m, 3H), 3.25-3.15 (m, 1H), 1.30-1.20 (m, 3H), |
| | 1.20-1.15 (m, 3H) |
| 1-495; | 8.93 (d, J = 2.1 Hz, 1H), 8.60 (dd, J = 4.5, 1.5 Hz, 1H), 8.13 (s, 1H), 8.02 (ddd, J = 8.1, 2.1, 1.5 Hz, 1H), |
| | 7.43 (dd, J = 8.1, 4.8 Hz, 1H), 4.30-3.40 (m, 1H), 2.96-2.78 (m, 1H), 2.63 (dd, J = 12.6, 6.0 Hz, 1H), |
| | 2.14 (s, 3H), 1.32 (d, J = 6.9 Hz, 3H) |
| 1-496; | 8.99-8.91 (m, 2H), 8.65-8.60 (m, 1H), 8.10-8.03 (m, 2H), 7.50-7.43 (m, 1H), 3.69-3.68 (m, 3H), |
| | 2.85-2.55 (m, 4H), 2.15-2.04 (m, 3H) |
| 1-497; | 8.92 (d, J = 2.7 Hz, 1H), 8.55 (dd, J = 4.8, 1.2 Hz, 1H), 8.8.00 (ddd, J = 8.4, 2.7, 1.2 Hz, 1H), 7.90 (s, 1H), |
| | 7.41 (dd, J = 8.4, 4.8 Hz, 1H), 3.56 (q, J = 7.2 Hz, 2H), 3.19 (s, 3H), 2.17 (s, 3H), 1.26 (t, J = 7.2 Hz, 3H) |
| 1-498; | 8.92 (d, J = 2.7 Hz, 1H), 8.60 (dd, J = 4.8, 1.8 Hz, 1H), 8.02 (ddd, J = 8.4, 2.7, 1.5 Hz, 1H), 7.92 (s, 1H), |
| | 7.55 (s, 1H), 7.44 (dd, J = 8.4, 4.8 Hz, 1H), 3.85 (q, J = 7.2 Hz, 2H), 2.49 (s, 3H), 1.27 (t, J = 7.2 Hz, 3H) |
| 1-500; | 8.92 (d, J = 2.7 Hz, 1H), 8.58 (dd, J = 4.5, 1.2 Hz, 1H), 8.01 (ddd, J = 8.7, 2.7, 1.2 Hz, 1H), 7.85 (s, 1H), |
| | 7.43 (dd, J = 8.7, 4.5 Hz, 1H), 3.62 (q, J = 7.2 Hz, 2H), 2.84 (s, 3H), 2.00-2.15 (m, 1H), 1.19 (t, J = 7.2 Hz, |
| | 3H), 0.65-0.75 (m, 2H), 0.55-0.65 (m, 2H) |
| 1-502; | 9.15-8.85 (m, 1H), 8.75-8.55 (m, 1H), 8.30-8.00 (m, 2H), 7.55-7.40 (m, 1H), 3.77 (q, J = 7.2 Hz, 2H), |
| | 1.52 (s, 9H), 1.26 (t, J = 7.2 Hz, 3H) |
| 1-503; | 8.97 (d, J = 2.7 Hz, 1H), 8.57 (dd, J = 4.8, 1.2 Hz, 1H), 8.02 (ddd, J = 8.7, 2.7, 1.2 Hz, 1H), 7.87 (brs, 1H), |
| | 7.41 (dd, J = 8.7, 4.8 Hz, 1H), 3.22 (s, 3H), 1.46 (brs, 9H) |
| 1-504; | 8.91 (d, J = 2.4 Hz, 1H), 8.59 (dd, J = 4.8, 1.5 Hz, 1H), 7.96 (ddd, J = 8.4, 2.4, 1.5 Hz, 1H), 7.76 (s, 1H), |
| | 7.43 (dd, J = 8.4, 4.8 Hz, 1H), 3.99 (q, J = 9.3 Hz, 2H), 3.57 (q, J = 7.2 Hz, 2H), 2.72 (s, 3H), 1.20 (t, J = 7.2 Hz, |
| | 3H) |
| 1-505; | 8.96 (d, J = 2.7 Hz, 1H), 8.62 (dd, J = 4.8, 1.2 Hz, 1H), 8.05 (ddd, J = 8.3, 2.7, 1.2 Hz, 1H), 8.01 (s, 1H), |
| | 7.47 (dd, J = 8.3, 4.8 Hz, 1H), 3.48 (d, J = 6.8 Hz, 2H), 2.80 (t, J = 7.4 Hz, 2H), 2.47 (t, J = 7.4 Hz, 2H), |
| | 2.07 (s, 3H), 1.05-0.90 (m, 1H), 0.55-0.40 (m, 2H), 0.25-0.10 (m, 2H) |
| 1-506; | (the isomer contained in a ratio of 10)9.04 (d, J = 2.7 Hz, 1H), 8.59 (dd, J = 4.8, 1.2 Hz, 1H), |
| | 8.31 (s, 1H), 8.10-8.00 (m, 1H), 7.50-7.30 (m, 1H), 4.10-3.05 (m, 5H), 2.61 (s, 3H), 1.23 (d, J = 6.8 Hz, |
| | 3H), 1.05-0.95 (m, 1H), 0.55-0.45 (m, 2H), 0.25-0.15 (m, 2H) |
| | (the isomer contained in a ratio of 4)9.00 (d, J = 2.7 Hz, 1H), 8.55 (dd, J = 4.8, 1.2 Hz, 1H), |
| | 8.25 (s, 1H), 8.10-8.00 (m, 1H), 7.50-7.30 (m, 1H), 4.10-3.05 (m, 5H), 2.61 (s, 3H), 1.23 (d, J = 6.8 Hz, |
| | 3H), 1.05-0.95 (m, 1H), 0.55-0.45 (m, 2H), 0.25-0.15 (m, 2H) |
| 1-507; | (the isomer contained in a ratio of 10)8.98 (d, J = 2.7 Hz, 1H), 8.62 (dd, J = 4.8, 1.2 Hz, 1H), |
| | 8.06 (ddd, J = 8.3, 2.7, 1.2 Hz, 1H), 8.13 (s, 1H), 7.46 (dd, J = 8.3, 4.8 Hz, 1H), 3.60-3.40 (m, 2H), |
| | 3.20-2.75 (m, 2H), 2.70-2.50 (m, 5H), 1.05-0.80 (m, 1H), 0.55-0.40 (m, 2H), 0.25-0.10 (m, 2H) |
| | (the isomer contained in a ratio of 3)8.91 (d, J = 2.7 Hz, 1H), 8.55 (dd, J = 4.8, 1.2 Hz, 1H), |
| | 7.99 (ddd, J = 8.3, 2.7, 1.2 Hz, 1H), 8.13 (s, 1H), 7.40 (dd, J = 8.3, 4.8 Hz, 1H), 3.60-3.40 (m, 2H), |
| | 3.20-2.75 (m, 2H), 2.70-2.50 (m, 5H), 1.05-0.80 (m, 1H), 0.55-0.40 (m, 2H), 0.25-0.10 (m, 2H) |
| 1-508; | (the isomer contained in a ratio of 10)8.98 (d, J = 2.7 Hz, 1H), 8.63 (dd, J = 4.8, 1.2 Hz, 1H), |
| | 8.25-8.00 (m, 2H), 7.44 (dd, J = 8.3, 4.8 Hz, 1H), 3.93 (q.J = 6.8 Hz, 1H), 3.90-3.30 (m, 2H), |
| | 2.90-2.70 (m, 2H), 1.50-1.20 (m, 6H), 1.10-0.95 (m, 1H), 0.55-0.45 (m, 2H), 0.25-0.15 (m, 2H) |
| | (the isomer contained in a ratio of 1)8.98 (d, J = 2.7 Hz, 1H), 8.63 (dd, J = 4.8, 1.2 Hz, 1H), |
| | 8.25-8.00 (m, 2H), 7.44 (dd, J = 8.3, 4.8 Hz, 1H), 3.93 (q.J = 6.8 Hz, 1H), 3.90-3.30 (m, 2H), |
| | 2.65-2.50 (m, 2H), 1.50-1.20 (m, 6H), 1.10-0.95 (m, 1H), 0.65-0.55 (m, 2H), 0.30-0.25 (m, 2H) |
| 1-509; | 9.02 (d, J = 2.7 Hz, 1H), 8.62 (dd, J = 4.8, 1.2 Hz, 1H), 8.23 (s, 1H), 8.05-8.00 (m, 1H), 7.43 (dd, J = 8.3, |
| | 4.8 Hz, 1H), 4.10-3.70 (m, 2H), 3.40-3.10 (m, 2H), 3.05-2.85 (m, 1H), 2.95 (s, 3H), 1.21 (d, J = 6.8 Hz, |
| | 3H), 1.05-0.95 (m, 1H), 0.55-0.45 (m, 2H), 0.25-0.15 (m, 2H) |
| 1-510; | 9.00-8.95 (m, 1H), 8.70-8.60 (m, 1H), 8.20-7.90 (m, 1H), 8.10 (s, 1H), 7.48 (dd, J = 8.3, 4.8 Hz, 1H), |
| | 3.60-3.30 (m, 4H), 2.96 (s, 3H), 2.80-2.70 (m, 2H), 1.05-0.90 (m, 1H), 0.65-0.40 (m, 2H), |
| | 0.30-0.10 (m, 2H) |
| 1-514; | 8.95 (d, J = 2.7 Hz, 1H), 8.60-8.55 (m, 1H), 8.10-7.95 (m, 2H), 7.44 (dd, J = 8.7, 4.8 Hz, 1H), |
| | 3.90-3.70 (m, 1H), 3.70-7.50 (m, 1H), 2.86 (dd, J = 12.6, 9.0 Hz, 1H), 2.70-2.60 (m, 1H), 2.55-2.35 (m, 3H), |
| | 1.20-1.10 (m, 9H) |
| 1-515; | (the isomer contained in a ratio of 2)9.05-9.00 (m, 1H), 8.65-8.55 (m, 1H), 8.30 (s, 1H), |
| | 8.05-7.95 (m, 1H), 7.45-7.35 (m, 1H), 3.40-3.25 (m, 1H), 3.30 (s, 3H), 3.20-3.05 (m, 1H), |
| | 2.80-2.50 (m, 3H), 1.34 (t, J = 7.5 Hz, 3H), 1.20 (d, J = 7.2 Hz, 3H) |
| | (the isomer contained in a ratio of 1)9.00-8.95 (m, 1H), 8.65-8.55 (m, 1H), 8.20 (s, 1H), |
| | 8.05-7.95 (m, 1H), 7.45-7.35 (m, 1H), 3.40-3.25 (m, 1H), 3.25 (s, 3H), 3.20-3.05 (m, 1H), |
| | 2.80-2.50 (m, 3H), 1.32 (t, J = 7.5 Hz, 3H), 1.25 (d, J = 7.2 Hz, 3H) |
| 1-516; | (the isomer contained in a ratio of 2)9.03 (d, J = 2.4 Hz, 1H), 8.65-8.55 (m, 1H), 8.25 (s, 1H), |
| | 8.05-7.95 (m, 1H), 7.45-7.35 (m, 1H), 4.20-4.05 (m, 1H), 3.50-3.30 (m, 1H), 3.30-3.00 (m, 2H), |
| | 2.80-2.50 (m, 3H), 1.34 (t, J = 7.5 Hz, 3H), 1.30-1.10 (m, 6H) |
| | (the isomer contained in a ratio of 1)8.98 (d, J = 2.4 Hz, 1H), 8.65-8.55 (m, 1H), 8.17 (s, 1H), |
| | 8.05-7.95 (m, 1H), 7.45-7.35 (m, 1H), 4.05-3.95 (m, 1H), 3.50-3.30 (m, 1H), 3.30-3.00 (m, 2H), |
| | 2.80-2.50 (m, 3H), 1.31 (t, J = 7.5 Hz, 3H), 1.30-1.10 (m, 6H) |
| 1-517; | 9.00 (d, J = 2.4 Hz, 1H), 8.60 (dd, J = 4.8, 1.8 Hz, 1H), 8.24 (s, 1H), 8.05-7.95 (m, 1H), 7.42 (dd, J = 7.8, |
| | 4.8 Hz, 1H), 3.76 (dd, J = 13.5, 11.1 Hz, 1H), 3.45-3.30 (m, 1H), 3.27 (s, 3H), 3.10-2.95 (m, 2H), |
| | 2.79 (dd, J = 13.5, 2.4 Hz, 1H), 1.41 (t, J = 7.5 Hz, 3H), 1.20 (d, J = 6.9 Hz, 3H) |
| 1-518; | 9.02 (d, J = 2.4 Hz, 1H), 8.61 (dd, J = 4.8, 1.5 Hz, 1H), 8.22 (s, 1H), 8.05-7.95 (m, 1H), 7.42 (dd, J = 8.7, |
| | 4.8 Hz, 1H), 4.15-4.00 (m, 1H), 3.74 (dd, J = 13.5, 11.1 Hz, 1H), 3.40-3.20 (m, 2H), 3.05-2.95 (m, 2H), |
| | 2.77 (dd, J = 13.5, 2.4 Hz, 1H), 1.41 (t, J = 7.2 Hz, 3H), 1.20 (d, J = 6.9 Hz, 3H), 1.16 (t, J = 7.2 Hz, 3H) |
| 1-520; | 8.94 (d, J = 2.7 Hz, 1H), 8.61 (dd, J = 4.8, 1.2 Hz, 1H), 8.04 (ddd, J = 8.1, 2.7, 1.2 Hz, 1H), 7.94 (s, 1H), |
| | 7.45 (dd, J = 8.1, 4.8 Hz, 1H), 3.70 (q, J = 7.2 Hz, 2H), 2.48 (t, J = 6.9 Hz, 2H), 2.26 (t, J = 7.2 Hz, 2H), |
| | 2.04 (s, 3H), 1.91 (tt, J = 7.2, 6.9 Hz, 2H), 1.14 (t, J = 7.2 Hz, 3H) |
| 1-523; | 9.03 (d, J = 2.4 Hz, 1H), 8.70-8.65 (m, 1H), 8.25-8.05 (m, 2H), 7.52 (dd, J = 8.4, 4.8 Hz, 1H), |
| | 4.35-4.05 (m, 1H), 3.35-3.15 (m, 1H), 2.90-2.55 (m, 2H), 2.55-2.40 (m, 1H), 2.03 (s, 3H), 1.32 (d, J = 6.9 Hz, |
| | 3H), 1.18 (t, J = 7.2 Hz, 3H) |
| 1-524; | 9.05-8.95 (m, 1H), 8.75-8.60 (m, 1H), 8.25-8.00 (m, 2H), 7.60-7.40 (m, 1H), 4.50-4.10 (m, 1H), |
| | 3.40-3.20 (m, 3H), 3.30-2.40 (m, 4H), 2.20-2.00 (m, 3H), 1.40-1.30 (m, 3H), 1.25-1.10 (m, 3H) |
| 1-525; | 9.02 (d, J = 2.4 Hz, 1H), 8.75-8.65 (m, 1H), 8.14 (s, 1H), 8.10-8.05 (m, 1H), 7.60-7.45 (m, 1H), |
| | 4.05-3.65 (m, 1H), 2.95-2.85 (m, 1H), 2.80-2.65 (m, 1H), 2.60-2.55 (m, 1H), 2.55-2.45 (m, 1H), 2.13 (s, |
| | 3H), 1.22 (d, J = 6.9 Hz, 3H), 1.30-1.15 (m, 3H) |
| 1-526; | 8.94 (d, J = 2.7 Hz, 1H), 8.50 (dd, J = 4.8, 1.5 Hz, 1H), 7.98 (ddd, J = 8.4, 2.7, 1.5 Hz, 1H), 7.88 (s, 1H), |
| | 7.41 (dd, J = 8.4, 4.8 Hz, 1H), 4.04 (s, 3H), 3.80-3.55 (m, 2H), 2.95-2.70 (m, 2H), 2.50-2.40 (m, 1H), |
| | 2.00 (s, 3H), 1.14 (t, J = 7.2 Hz, 3H), 1.13 (d, J = 6.9 Hz, 3H) |
| 1-527; | 8.96 (d, J = 2.7 Hz, 1H), 8.61 (dd, J = 4.8, 1.2 Hz, 1H), 8.04 (ddd, J = 8.1, 2.7, 1.2 Hz, 1H), 7.98 (s, 1H), |
| | 7.45 (dd, J = 8.1, 4.8 Hz, 1H), 3.77-3.62 (m, 2H), 2.85-2.65 (m, 2H), 2.55 (s, 3H), 2.35 (t, J = 6.9 Hz, 2H), |
| | 2.16-2.02 (m, 2H), 1.15 (t, J = 7.2 Hz, 3H) |
| 1-528; | 8.95 (d, J = 2.7 Hz, 1H), 8.61 (dd, J = 4.8, 1.2 Hz, 1H), 8.02 (ddd, J = 8.1, 2.7, 1.2 Hz, 1H), 7.97 (s, 1H), |
| | 7.44 (dd, J = 8.1, 4.8 Hz, 1H), 3.70 (q, J = 7.2 Hz, 2H), 3.10 (t, J = 7.2 Hz, 2H), 2.90 (s, 3H), 2.38 (t, J = 6.9 Hz, |
| | 2H), 2.15 (tt, J = 7.2, 6.9 Hz, 2H), 1.15 (t, J = 7.2 Hz, 3H) |
| 1-530; | 8.91 (d, J = 2.7 Hz, 1H), 8.60 (dd, J = 4.8, 1.5 Hz, 1H), 7.96 (ddd, J = 8.4, 2.7, 1.5 Hz, 1H), 7.78 (s, 1H), |
| | 7.43 (dd, J = 8.4, 4.8 Hz, 1H), 3.94 (q, J = 9.3 Hz, 2H), 3.57 (q, J = 7.2 Hz, 2H), 3.08 (q, J = 7.2 Hz 2H), |
| | 1.20 (t, J = 7.2 Hz, 3H), 0.97 (t, J = 7.2 Hz, 3H) |
| 1-531; | 8.86 (d, J = 2.4 Hz, 1H), 8.62 (dd, J = 4.8, 1.2 Hz, 1H), 7.93 (ddd, J = 8.1, 2.4, 1.2 Hz, 1H), |
| | 7.60-7.30 (m, 4H), 6.96 (d, J = 8.7 Hz, 1H), 6.93 (d, J = 8.7 Hz, 1H), 3.90-3.70 (m, 1H), 3.70-3.50 (m, 1H), 3.62 (q, |
| | J = 6.9 Hz, 1H), 1.41 (d, J = 6.9 Hz, 3H), 1.15 (t, J = 7.2 Hz, 3H) |
| 1-532; | 8.79 (d, J = 2.4 Hz, 1H), 8.61 (dd, J = 4.8, 1.2 Hz, 1H), 7.92 (ddd, J = 8.1, 2.4, 1.2 Hz, 1H), 7.49 (s, 1H), |
| | 7.43 (dd, J = 8.1, 4.8 Hz, 1H), 7.25-6.85 (m, 4H), 3.90-3.70 (m, 1H), 3.72 (q, J = 6.9 Hz, 1H), |
| | 3.70-3.55 (m, 1H), 1.49 (d, J = 6.9 Hz, 3H), 1.15 (t, J = 7.2 Hz, 3H) |
| 1-533; | 8.85 (d, J = 2.7 Hz, 1H), 8.62 (dd, J = 4.8, 1.2 Hz, 1H), 7.98 (ddd, J = 8.1, 2.7, 1.2 Hz, 1H), 7.67 (s, 1H), |
| | 7.50-7.40 (m, 2H), 7.30-7.20 (m, 1H), 7.06 (ddd, J = 7.5, 7.5, 1.2 Hz, 1H), 6.96 (ddd, J = 8.7, 7.5, 1.2 Hz, |
| | 1H), 3.90-3.70 (m, 1H), 3.73 (q, J = 6.9 Hz, 1H), 3.65-3.45 (m, 1H), 1.45 (d, J = 6.9 Hz, 3H), |
| | 1.13 (t, J = 7.2 Hz, 3H) |
| 1-534; | 8.73 (d, J = 2.7 Hz, 1H), 8.61 (dd, J = 4.8, 1.2 Hz, 1H), 7.91 (ddd, J = 8.1, 2.7, 1.2 Hz, 1H), 7.44 (ddd, J = 8.1, |
| | 2.4, 1.2 Hz, 1H), 7.30-7.20 (m, 2H), 7.06 (s, 1H), 7.04 (d, J = 7.5 Hz, 2H), 3.90-3.70 (m, 1H), |
| | 3.65 (q, J = 6.9 Hz, 1H), 3.65-3.45 (m, 1H), 2.30 (s, 3H), 1.46 (d, J = 6.9 Hz, 3H), 1.14 (t, J = 7.2 Hz, 3H) |
| 1-535; | 8.81 (d, J = 2.1 Hz, 1H), 8.61 (dd, J = 4.8, 1.2 Hz, 1H), 7.60-7.70 (m, 1H), 7.7-7.60 (m, 1H), |
| | 7.50-7.30 (m, 5H), 3.90-3.55 (m, 3H), 1.51 (d, J = 7.2 Hz, 3H), 1.17 (t, J = 7.2 Hz, 3H) |
| 1-536; | 8.91 (d, J = 2.7 Hz, 1H), 8.61 (dd, J = 4.8, 1.5 Hz, 1H), 7.96 (ddd, J = 8.4, 2.7, 1.5 Hz, 1H), 7.79 (s, 1H), |
| | 7.43 (dd, J = 8.4, 4.8 Hz, 1H), 4.22 (q, J = 9.3 Hz, 2H), 4.16 (s, 2H), 3.59 (q, J = 6.9 Hz, 2H), 2.07 (s, 3H), |
| | 1.21 (t, J = 6.9 Hz, 3H) |
| 1-539; | (the isomer contained in a ratio of 78)8.95 (d, J = 2.4 Hz, 1H), 8.61 (dd, J = 4.8, 1.2 Hz, 1H), |
| | 8.04 (ddd, J = 8.1, 2.4, 1.2 Hz, 1H), 7.94 (s, 1H), 7.45 (dd, J = 8.1, 4.8 Hz, 1H), 6.90 (dd, J = 15.0, 7.5 Hz, |
| | 1H), 5.86 (d, J = 15.0 Hz, 1H), 3.76 (q, J = 7.2 Hz, 2H), 3.16 (d, J = 7.5 Hz, 2H), 1.96 (s, 3H), |
| | 1.28-1.11 (m, 3H) |
| | (the isomer contained in a ratio of 22)8.95 (d, J = 2.4 Hz, 1H), 8.61 (dd, J = 4.8, 1.2 Hz, 1H), |
| | 8.05-7.99 (m, 1H), 7.91 (s, 1H), 7.45 (dd, J = 8.1, 4.8 Hz, 1H), 5.86 (d, J = 15.0 Hz, 1H), 5.50-5.38 (m, 1H), |
| | 3.69 (q, J = 7.5 Hz, 2H), 3.00 (d, J = 7.2 Hz, 2H), 1.96 (s, 3H), 1.28-1.11 (m, 3H) |
| 1-541; | 9.50-8.50 (m, 1H), 8.50-7.50 (m, 3H), 7.00-6.90 (m, 1H), 4.69 (q, J = 9.3 Hz, 2H), 3.92 (q, J = 6.9 Hz, |
| | 2H), 2.87 (s, 3H), 1.29 (t, J = 6.9 Hz, 3H) |
| 1-542; | 8.98 (d, J = 2.4 Hz, 1H), 8.66 (d, J = 4.5 Hz, 1H), 8.20 (s, 1H), 8.04 (ddd, J = 8.4, 2.4, 0.9 Hz, 1H), |
| | 7.48 (dd, J = 8.4, 4.5 Hz, 1H), 4.61 (brs, 2H), 2.80 (t, J = 7.2 Hz, 2H), 2.53 (t, J = 7.2 Hz, 2H), 2.08 (s, 3H) |
| 1-543; | 9.00 (d, J = 2.4 Hz, 1H), 8.66 (d, J = 4.8 Hz, 1H), 8.26 (s, 1H), 8.04 (m, 1H), 7.47 (dd, J = 8.4, 4.8 Hz, 1H), |
| | 4.60 (brs, 2H), 3.20-3.11 (m, 1H), 2.95-2.85 (m, 1H), 2.79-2.74 (m, 2H), 2.60 (s, 3H) |
| 1-544; | 9.00 (m, 1H), 8.67 (m, 1H), 8.27 (s, 1H), 8.37 (ddd, J = 8.1, 2.4, 0.9 Hz, 1H), 7.48 (dd, J = 8.1, 4.8 Hz, 1H), |
| | 4.59 (brs, 2H), 3.44 (t, J = 6.9 Hz, 2H), 2.98 (s, 3H), 2.80 (t, J = 6.9 Hz, 2H) |
| 1-545; | 8.95 (d, J = 2.7 Hz, 1H), 8.63 (dd, J = 4.8, 1.5 Hz, 1H), 8.05 (ddd, J = 8.1, 2.7, 1.5 Hz, 1H), 8.01 (s, 1H), |
| | 7.46 (dd, J = 8.1, 4.8 Hz, 1H), 3.26 (s, 3H), 2.87 (dd, J = 12.6, 9.0 Hz, 1H), 2.85-2.65 (m, 1H), 2.48 (dd, |
| | J = 12.6, 5.1 Hz, 1H), 2.03 (s, 3H), 1.18 (d, J = 6.6 Hz, 3H) |
| 1-546; | 8.90 (d, J = 2.4 Hz, 1H), 8.60 (dd, J = 4.5, 1.5 Hz, 1H), 8.02 (ddd, J = 8.1, 2.4, 1.5 Hz, 1H), 7.99 (s, 1H), |
| | 7.43 (dd, J = 8.1, 4.5 Hz, 1H), 3.70 (s, 3H), 3.54 (s, 2H), 3.35 (s, 3H), 1.92 (s, 3H) |
| 1-568; | (the isomer contained in a ratio of 3)9.00-8.90 (m, 1H), 8.65-8.55 (m, 1H), 8.40 (s, 1H), |
| | 8.15-7.95 (m, 1H), 7.50-7.40 (m, 1H), 5.55-5.40 (m, 1H), 5.20-5.00 (m, 1H), 3.70-3.45 (m, 2H), |
| | 3.35-3.15 (m, 1H), 2.53 (s, 3H), 1.27 (d, J = 7.2 Hz, 3H) |
| | (the isomer contained in a ratio of 2) |
| | 9.00-8.90 (m, 1H), 8.65-8.55 (m, 1H), 8.35 (s, 1H), 8.15-7.95 (m, 1H), 7.50-7.40 (m, 1H), |
| | 5.55-5.40 (m, 1H), 5.20-5.00 (m, 1H), 3.70-3.45 (m, 2H), 3.35-3.15 (m, 1H), 2.68 (s, 3H), 1.29 (d, J = 7.2 Hz, |
| | 3H) |
| 1-569; | 9.03 (d, J = 2.7 Hz, 1H), 8.70-8.60 (m, 1H), 8.40 (s, 1H), 8.15-7.95 (m, 1H), 7.45 (dd, J = 4.8, 8.1 Hz, 1H), |
| | 5.10-4.90 (m, 1H), 4.35-4.10 (m, 2H), 3.85-3.70 (m, 1H), 3.40-3.30 (m, 1H), 2.97 (s, 3H), |
| | 1.25 (d, J = 7.2 Hz, 3H) |
| 1-573; | 8.90 (d, J = 2.7 Hz, 1H), 8.59 (dd, J = 4.8, 1.2 Hz, 1H), 7.94 (ddd, J = 8.3, 2.7, 1.2 Hz, 1H), 7.77 (s, 1H), |
| | 7.43 (dd, J = 8.3, 4.8 Hz, 1H), 4.35 (s, 2H), 4.03 (q, J = 9.2 Hz, 2H), 3.61 (q, J = 7.1 Hz, 2H), 3.20 (s, 3H), |
| | 1.20 (t, J = 7.1 Hz, 3H) |
| 1-581; | (the isomer contained in a ratio of 7)9.04 (d, J = 3.0 Hz, 1H), 8.60 (dd, J = 4.8, 1.5 Hz, 1H), |
| | 8.25 (s, 1H), 8.10-7.95 (m, 1H), 7.50-7.35 (m, 1H), 3.35-3.10 (m, 2H), 3.29 (s, 3H), 2.75-2.55 (m, 1H), |
| | 2.60 (s, 3H), 1.23 (d, J = 6.6 Hz, 3H) |
| | (the isomer contained in a ratio of 5) |
| | 8.99 (d, J = 2.4 Hz, 1H), 8.61 (dd, J = 4.8, 1.5 Hz, 1H), 8.18 (s, 1H), 8.10-7.95 (m, 1H), 7.50-7.35 (m, 1H), |
| | 3.35-3.10 (m, 2H), 3.26 (s, 3H), 2.75-2.55 (m, 1H), 2.60 (s, 3H), 1.27 (d, J = 6.6 Hz, 3H) |
| 1-582; | 9.01 (d, J = 2.7 Hz, 1H), 8.62 (dd, J = 4.8, 1.5 Hz, 1H), 8.20 (s, 1H), 8.01 (ddd, J = 8.4, 2.7, 1.5 Hz, 1H), |
| | 7.43 (dd, J = 8, 4.8 Hz, 1H), 3.82 (dd, J = 14.1, 11.4 Hz, 1H), 3.40-3.30 (m, 1H), 3.28 (s, 3H), 2.95 (s, 3H), |
| | 2.89 (dd, J = 14.1, 2.7 Hz, 1H), 1.22 (d, J = 6.9 Hz, 3H) |
| 1-583; | 8.97 (d, J = 2.7 Hz, 1H), 8.58 (dd, J = 4.8, 1.2 Hz, 1H), 8.20 (s, 1H), 8.00 (ddd, J = 8.4, 2.7, 1.2 Hz, 1H), |
| | 7.41 (dd, J = 8.4, 4.8 Hz, 1H), 4.06 (d, J = 12.6 Hz, 1H), 3.95 (d, J = 12.6 Hz, 1H), 3.76 (s, 3H), 3.34 (s, 3H), |
| | 2.60 (s, 3H) |
| 1-584; | 8.96 (d, J = 2.7 Hz, 1H), 8.59 (dd, J = 4.8, 1.5 Hz, 1H), 8.07 (s, 1H), 7.98 (ddd, J = 8.4, 2.7, 1.5 Hz, 1H), |
| | 7.42 (dd, J = 8.4, 4.8 Hz, 1H), 4.47 (s, 2H), 3.80 (s, 3H), 3.37 (s, 3H), 2.88 (s, 3H) |
| 1-588; | 8.93 (d, J = 2.4 Hz, 1H), 8.63 (dd, J = 5.1, 1.2 Hz, 1H), 8.06 (ddd, J = 8.4, 2.4, 1.2 Hz, 1H), 7.98 (s, 1H), |
| | 7.47 (dd, J = 8.4, 5.1 Hz, 1H), 4.72 (d, J = 10.2 Hz, 1H), 4.05-3.45 (m, 2H).3.35-2.50 (m, 5H), 1.23 (d, |
| | J = 6.9 Hz, 3H), 1.18 (t, J = 7.2 Hz, 3H) |
| 1-590; | 8.90-8.80 (m, 1H), 8.52 (s, 1H), 8.50-8.40 (m, 1H), 8.00-7.90 (m, 1H), 7.85-7.75 (m, 3H), |
| | 7.70-7.65 (m, 1H), 7.30-7.25 (m, 1H), 7.15-7.10 (m, 1H), 3.83 (q, J = 7.2 Hz, 2H), 3.54 (s, 3H), 1.19 (t, J = 7.2 Hz, |
| | 3H) |
| 1-596; | 9.02 (d, J = 2.7 Hz, 1H), 8.65-8.55 (m, 1H), 8.19 (s, 1H), 8.05-7.95 (m, 1H), 7.42 (dd, J = 4.8, 8.1 Hz, 1H), |
| | 4.15-3.95 (m, 1H), 3.60-3.30 (m, 2H), 3.25-3.10 (m, 1H), 2.85 (s, 6H), 2.75-2.60 (m, 1H), |
| | 1.21 (d, J = 7.2 Hz, 3H), 1.17 (t, J = 7.2 Hz, 3H) |
| 1-598; | 8.84 (d, J = 2.7 Hz, 1H), 8.56 (dd, J = 4.8, 1.2 Hz, 1H), 8.02 (s, 1H), 7.96 (ddd, J = 8.4, 2.7, 1.2 Hz, 1H), |
| | 7.45-7.41 (m, 1H), 5.50 (m, 1H), 4.14 (d, J = 6.0 Hz, 2H), 3.68 (q, J = 7.2 Hz, 2H), 1.16 (t, J = 7.2 Hz, 3H) |
| 1-599; | 8.95 (d, J = 2.1 Hz, 1H), 8.61 (dd, J = 4.8, 1.5 Hz, 1H), 8.42 (ddd, J = 4.8, 1.5, 0.9 Hz, 1H), |
| | 8.07-8.02 (m, 2H), 7.64 (ddd, J = 7.8, 7.8, 2.1 Hz, 1H), 7.45 (ddd, J = 8.1, 4.8, 0.9 Hz, 1H), 7.32-7.28 (m, 1H), |
| | 7.18-7.10 (m, 1H), 5.67 (m, 1H), 4.52 (d, J = 5.7 Hz, 2H), 3.72 (q, J = 7.2 Hz, 2H), 1.17 (t, J = 7.2 Hz, 3H) |
| 1-601; | 8.74 (d, J = 2.7 Hz, 1H), 8.57 (d, J = 4.8 Hz, 1H), 7.95-7.85 (m, 1H), 7.50-7.20 (m, 7H), 5.65 (brs, 1H), |
| | 4.16 (brs, 1H), 2.92 (dd, J = 12.6, 9.0 Hz, 1H), 2.80-2.70 (m, 1H), 2.51 (dd, J = 12.6, 5.1 Hz, 1H), |
| | 2.00 (s, 3H), 1.21 (d, J = 6.3 Hz, 3H) |
| 1-602; | 8.74 (d, J = 2.4 Hz, 1H), 8.58 (dd, J = 4.5, 1.2 Hz, 1H), 7.91 (ddd, J = 8.4, 2.4, 1.2 Hz, 1H), |
| | 7.45-7.20 (m, 7H), 4.90 (brs, 2H), 2.85 (t, J = 7.2 Hz, 2H), 2.51 (t, J = 7.2 Hz, 2H), 2.08 (s, 3H) |
| 1-603; | 8.85 (d, J = 2.4 Hz, 1H), 8.59 (dd, J = 4.8, 1.5 Hz, 1H), 8.31 (s, 1H), 7.99 (ddd, J = 8.1, 2.4, 1.5 Hz, 1H), |
| | 7.43 (dd, J = 8.1, 4.8 Hz, 1H), 5.91 (s, 1H), 5.45-5.05 (m, 1H), 4.60-4.25 (m, 1H), 3.96 (s, 6H), |
| | 3.48 (q, J = 6.9 Hz, 1H), 2.68-2.50 (m, 2H), 1.51 (d, J = 6.9 Hz, 3H), 1.14 (t, J = 7.5 Hz, 3H) |
| 1-604; | 8.76 (d, J = 2.7 Hz, 1H), 8.56 (dd, J = 4.8, 1.2 Hz, 1H), 7.89 (ddd, J = 8.4, 2.7, 1.2 Hz, 1H), 7.59 (s, 1H), |
| | 7.39 (dd, J = 8.4, 4.8 Hz, 1H), 7.36-7.19 (m, 5H), 5.80-5.20 (m, 1H), 4.40-3.90 (m, 1H), |
| | 3.46-3.27 (m, 1H), 2.71-2.50 (m, 2H), 1.51 (d, J = 6.9 Hz, 3H), 1.17 (t, J = 7.5 Hz, 3H) |
| 2-003; | 8.94 (d, J = 2.7 Hz, 1H), 8.57 (dd, J = 4.8, 1.2 Hz, 1H), 8.15 (s, 1H), 7.99 (ddd, J = 8.1, 2.7, 1.2 Hz, 1H), |
| | 7.42 (dd, J = 8.1, 4.8 Hz, 1H), 6.42 (dd, J = 16.5, 9.9 Hz, 1H), 6.27 (d, J = 16.5 Hz, 1H), 6.09 (d, J = 9.9 Hz, |
| | 1H), 3.28 (s, 3H) |
| 3-006; | (the isomer contained in a ratio of 2)8.89 (d, J = 2.1 Hz, 1H), 8.54 (dd, J = 4.8, 1.2 Hz, 1H), |
| | 8.01-7.99 (m, 1H), 7.59 (s, 1H), 7.40 (dd, J = 8.4, 4.8 Hz, 1H), 4.12 (q, J = 7.2 Hz, 1H), 2.71-2.53 (m, 2H), |
| | 2.42 (s, 3H), 1.51 (d, J = 7.2 Hz, 3H), 1.19 (t, J = 7.2 Hz, 3H) |
| | (the isomer contained in a ratio of 1)8.92-8.89 (m, 1H), 8.54 (dd, J = 4.8, 1.2 Hz, 1H), |
| | 8.01-7.99 (m, 1H), 7.93 (s, 1H), 7.40 (dd, J = 8.4, 4.8 Hz, 1H), 3.89 (q, J = 6.9 Hz, 1H), 2.71-2.53 (m, 2H), |
| | 2.65 (s, 3H), 1.69 (d, J = 6.9 Hz, 3H), 1.27 (t, J = 7.2 Hz, 3H) |
| 3-009; | (the isomer contained in a ratio of 2)8.95 (d, J = 2.7 Hz, 1H), 8.55-8.53 (m, 1H), |
| | 8.05-8.01 (m, 1H), 7.69 (s, 1H), 7.50 (s, 1H), 7.43-7.39 (m, 1H), 4.27 (q, J = 7.2 Hz, 1H), 2.54 (q, J = 7.2 Hz, 2H), |
| | 2.38 (s, 3H), 1.52 (d, J = 7.2 Hz, 3H), 1.19 (t, J = 7.2 Hz, 3H) |
| | (the isomer contained in a ratio of 1)8.97 (d, J = 3.0 Hz, 1H), 8.55-8.53 (m, 1H), |
| | 8.05-8.01 (m, 1H), 8.00 (s, 1H), 7.84 (s, 1H), 7.43-7.39 (m, 1H), 3.89 (q, J = 7.2 Hz, 1H), 2.64 (q, J = 7.2 Hz, 2H), |
| | 2.63 (s, 3H), 1.66 (d, J = 7.2 Hz, 3H), 1.27 (t, J = 7.2 Hz, 3H) |
| 3-010; | (the isomer contained in a ratio of 4)8.98-8.70 (m, 2H), 8.60-8.50 (m, 1H), 8.10-7.95 (m, 1H), |
| | 7.91 (s, 1H), 7.50-7.35 (m, 1H), 4.09 (s, 3H), 3.40-3.25 (m, 1H), 3.00-2.50 (m, 2H), 2.17 (s, 3H), |
| | 1.32 (d, J = 7.2 Hz, 3H) |
| | (the isomer contained in a ratio of 1)8.98-8.70 (m, 2H), 8.60-8.50 (m, 1H), 8.10-7.95 (m, 1H), |
| | 7.91 (s, 1H), 7.50-7.35 (m, 1H), 3.79 (s, 3H), 3.00-2.50 (m, 3H), 2.13-2.00 (m, 3H), |
| | 1.37-1.23 (m, 3H) |
| 3-012; | (the isomer contained in a ratio of 77)8.97 (s, 1H), 8.92 (d, J = 2.7 Hz, 1H), 8.56 (dd, J = 4.8, |
| | 1.2 Hz, 1H), 8.01 (ddd, J = 8.1, 2.7, 1.2 Hz, 1H), 7.90 (s, 1H), 7.41 (dd, J = 8.1, 4.8 Hz, 1H), 4.15 (s, 3H), |
| | 4.15-4.02 (m, 1H), 2.79-2.57 (m, 2H), 1.57 (d, J = 7.2 Hz, 3H), 1.31-1.22 (m, 3H) |
| | (the isomer contained in a ratio of 23)8.92 (d, J = 2.7 Hz, 1H), 8.62 (s, 1H), 8.56 (dd, J = 4.8, |
| | 1.2 Hz, 1H), 8.01 (ddd, J = 8.1, 2.7, 1.2 Hz, 1H), 7.90 (s, 1H), 7.41 (dd, J = 8.1, 4.8 Hz, 1H), |
| | 4.15-4.02 (m, 1H), 3.78 (s, 3H), 2.79-2.57 (m, 2H), 1.50 (d, J = 7.5 Hz, 3H), 1.31-1.22 (m, 3H) |
| 5-003; | 8.80 (d, J = 2.4 Hz, 1H), 8.67 (dd, J = 4.8, 1.2 Hz, 1H), 7.87 (ddd, J = 8.1, 2.4, 1.2 Hz, 1H), 7.61 (brs, 1H), |
| | 7.48 (dd, J = 8.1, 4.8 Hz, 1H), 4.09-3.79 (m, 1H), 3.57-3.27 (m, 1H), 2.93-2.79 (m, 1H), |
| | 2.77-2.57 (m, 1H), 2.39 (dd, J = 12.9, 5.1 Hz, 1H), 2.33 (brs, 3H), 1.99 (s, 3H), 1.14 (t, J = 7.2 Hz, 3H), 1.10 (d, J = 6.9 Hz, |
| | 3H) |
| 5-004; | 8.78 (d, J = 2.4 Hz, 1H), 8.66 (dd, J = 4.8, 1.2 Hz, 1H), 7.90-7.82 (m, 1H), 7.76-7.52 (m, 1H), 7.47 (dd, |
| | J = 8.1, 4.8 Hz, 1H), 4.14-3.81 (m, 1H), 3.55-3.21 (m, 2H), 2.61-2.49 (m, 2H), 2.31 (brs, 3H), 1.43 (d, |
| | J = 6.9 Hz, 3H), 1.15 (t, J = 7.2 Hz, 3H), 1.14 (t, J = 7.2 Hz, 3H) |
| 5-005; | 8.91 (d, J = 2.4 Hz, 1H), 8.67 (dd, J = 4.8, 1.5 Hz, 1H), 7.95 (ddd, J = 8.4, 2.4, 1.5 Hz, 1H), 7.68 (s, 1H), |
| | 7.46 (dd, J = 8.4, 4.8 Hz, 1H), 3.61 (q, J = 7.2 Hz, 2H), 1.45 (s, 9H), 1.18 (t, J = 7.2 Hz, 3H) |
| 5-007; | 8.99 (d, J = 2.4 Hz, 1H), 8.70 (dd, J = 4.8, 1.5 Hz, 1H), 8.04 (ddd, J = 8.1, 2.4, 1.5 Hz, 1H), 7.48 (dd, J = 8.1, |
| | 4.8 Hz, 1H), 4.30-4.15 (m, 1H), 3.85-3.70 (m, 1H), 3.40-3.30 (m, 1H), 2.70-2.50 (m, 4H), 1.52 (d, |
| | 6.9 Hz, 3H), 1.07 (t, J = 7.5 Hz, 3H), 1.25-1.15 (m, 6H) |
| 6-001; | 9.21 (s, 1H), 9.10 (s, 2H), 8.13 (s, 1H), 3.90-3.70 (m, 1H), 3.70-3.50 (s, 1H), 3.25 (q, J = 6.9 Hz, 1H), |
| | 2.70-2.50 (m, 2H), 1.47 (d, J = 6.9 Hz, 3H), 1.17 (t, J = 7.5 Hz, 3H), 1.16 (t, J = 7.5 Hz, 3H) |
| 6-002; | 8.72 (d, J = 1.8 Hz, 1H), 8.45-8.40 (m, 1H), 8.08 (s, 1H), 7.90-7.85 (m, 1H), 3.90-3.70 (m, 1H), |
| | 3.70-3.50 (m, 1H), 3.29 (q, J = 6.9 Hz, 1H), 2.65-2.50 (m, 2H), 2.45 (s, 3H), 1.47 (d, J = 6.9 Hz, 3H), 1.16 (t, |
| | J = 7.2 Hz, 3H), 1.15 (t, J = 7.5 Hz, 3H) |
| 6-003; | 8.49 (d, J = 1.8 Hz, 1H), 8.31 (d, J = 2.4 Hz, 1H), 8.09 (s, 1H), 7.60 (dd, J = 2.4, 1.8 Hz, 1H), 3.86 (s, 3H), |
| | 3.85-3.70 (m, 1H), 3.70-3.55 (m, 1H), 3.29 (q, J = 6.9 Hz, 1H), 2.65-2.50 (m, 2H), 1.47 (d, J = 6.9 Hz, |
| | 3H), 1.17 (t, J = 7.5 Hz, 3H), 1.16 (t, J = 7.2 Hz, 3H) |
| 6-010; | 8.35-8.25 (m, 1H), 8.10-8.00 (m, 1H), 8.00-7.90 (m, 1H), 7.80-7.70 (m, 1H), 7.40-7.35 (m, 1H), |
| | 3.91 (brs, 2H), 3.28 (s, 3H), 1.75-1.60 (m, 1H), 1.15-1.00 (m, 2H), 0.75-0.65 (m, 2H) |
| 7-002; | 8.79 (d, J = 2.7 Hz, 1H), 8.04 (s, 1H), 7.90 (dd, J = 8.4, 2.7 Hz, 1H), 7.28 (d, J = 8.4 Hz, 1H), |
| | 3.85-3.70 (m, 1H), 3.70-3.55 (m, 1H), 3.30 (q, J = 6.9 Hz, 1H), 2.62 (s, 3H), 2.60-2.50 (m, 2H), 1.46 (d, J = 6.9 Hz, |
| | 3H), 1.16 (t, J = 7.2 Hz, 3H), 1.15 (t, J = 7.5 Hz, 3H) |
| 7-003; | 8.57 (s, 1H), 8.55 (d, J = 4.8 Hz, 1H), 7.78 (s, 1H), 7.29 (d, J = 4.8 Hz, 1H), 3.90-3.75 (m, 1H), |
| | 3.70-3.50 (m, 1H), 3.31 (q, J = 6.9 Hz, 1H), 2.70-2.50 (m, 2H), 2.37 (s, 3H), 1.48 (d, J = 6.9 Hz, 3H), 1.17 (t, J = 7.5 Hz, |
| | 3H), 1.16 (t, J = 7.2 Hz, 3H) |
| 7-004; | 8.61 (dd, J = 4.8, 1.8 Hz, 1H), 7.78 (s, 1H), 7.70 (dd, J = 8.4, 1.8 Hz, 1H), 7.30 (dd, J = 8.4, 4.8 Hz, 1H), |
| | 3.95-3.70 (m, 1H), 3.7-3.50 (m, 1H), 3.31 (q, J = 6.9 Hz, 1H), 2.58 (q, J = 7.5 Hz, 2H), 2.57 (s, 3H), |
| | 1.48 (d, J = 6.9 Hz, 3H), 1.18 (t, J = 7.5 Hz, 3H), 1.16 (t, J = 7.2 Hz, 3H) |

TABLE 12

| No. | Proton NMR chemical shift (in DMSO-d6): σ(ppm) |
|---|---|
| 1-004; | 9.02(d, J = 2.7 Hz, 1H), 8.80(s, 1H), 8.46(d, J = 4.8 Hz, 1H), 8.43(s, 1H), |
| | 8.19-8.13(m, 1H), 7.76(s, 1H), 7.50(dd, J = 4.8, 8.7 Hz, 1H), 6.95(t, J = |
| | 6.6 Hz, 1H), 3.97-3.83(m, 2H) |
| 1-009; | 10.62(s, 1H), 9.06(d, J = 2.4 Hz, 1H), 8.70(s, 1H), 8.47(dd, J = 1.2, 4.8 |
| | Hz, 1H), 8.20(ddd, J = 1.2, 2.4, 8.1 Hz, 1H), 7.91(s, 1H), 7.82(d, J = 8.4 |
| | Hz, 2H), 7.77(d, J = 8.4 Hz, 2H), 7.62(d, J = 16.0 Hz, 1H), 7.50(dd, J = |
| | 4.8, 8.1 Hz, 1H), 6.88(d, J = 16.0 Hz, 1H) |

The proton NMR chemical shifts in Tables 11 and 12 were measured by using Me₄Si (tetramethylsilane) as the standard compound at 300 MHz.

Compounds of the present invention having a substituent having an asymmetric carbon were resolved to optically active substances by high performance liquid chromatography using an optically active column by the method described in detail below. However, in the present invention, there is no restriction to the method.

As a high performance liquid chromatograph, 10AVP system, manufactured by Shimadzu Corporation, was used.

The optical resolution was carried out under the following conditions.

Flow rate: 6.0 ml/min Oven temp.: 30° C.

Mobile phase: n-hexane:ethanol=7:3 (volume ratio)

Column: CHIRALPAK AD-H (inner diameter 20 mm, length 250 mm, particle diameter 5 μm), manufactured by Dicel Corporation

Wavelength: 254 nm

The method will be described in reference to Compound No. 1-048 of the present invention. Compound No. 1-048 (400 mg) was dissolved in 4 ml of a solvent mixture of n-hexane:ethanol=1:1 (volume ratio). The resulting solution was subjected to optical resolution by high performance liquid chromatography under the conditions described above in forty 0.1-ml aliquots, and the fractions corresponding to peaks with retention times of 18.4 min and 21.1 min were collected. The solvent was evaporated from each fractions under reduced pressure to obtain 132.5 mg of white crystals (peak area percentage 99%, [α]_{D}^{23.9}−10.00° (CHCl₃, c=0.510)} from the fraction having a retention time of 18.4 min and 128.2 mg of white crystals {peak area percentage 92%, [α]_{D}^{24.1}+8.95° (CHCl₃, c=0.503)} from the fraction having a retention time of 21.1 min.

### TEST EXAMPLES

Now, usefulness of the compounds of the present invention as pesticides will be described in detail by referring to the following Test Examples, but the present invention is by no means restricted thereto.

### Test Example 1

### Insecticidal Test on Nilaparvata lugens

10% emulsifiable concentrates (or 10% wettable powders) of compounds of the present invention were diluted with water containing a spreader to obtain 500 ppm solutions. Rice sheaths were soaked in the solutions for about 10 seconds, dried in air and put in test tubes. In each tube, five 2nd-instar nymphs of Nilaparvata lugens were released, and the tubes were capped with sponge and placed in an incubator at 25° C. 6 Days after, dead insects were counted, and the mortality (%) (the number of dead insects÷the number of released insects×100) was calculated. The test was carried out in duplicate.

Among the compounds tested, the following compounds showed a mortality of at least 90%.

Compounds Nos. 1-002, 1-007, 1-008, 1-011, 1-012, 1-013, 1-014, 1-018, 1-019, 1-020, 1-021, 1-022, 1-023, 1-026, 1-028, 1-031 to 1-054, 1-056 to 1-070, 1-073 to 1-080, 1-082 to 1-089, 1-093 to 1-107, 1-109 to 1-113, 1-115 to 1-124, 1-126, 1-130, 1-131, 1-134, 1-135, 1-140 to 1-144, 1-146 to 1-155, 1-157, 1-158, 1-162 to 1-173, 1-177, 1-178, 1-179, 1-184, 1-188, 1-189, 1-190, 1-193 to 1-199, 1-201 to 1-210, 1-212, 1-213, 1-216, 1-220, 1-221, 1-222, 1-223, 1-225 to 1-229, 1-231 to 1-244, 1-247, 1-248, 1-253, 1-254, 1-255, 1-258 to 1-271, 1-273, 1-275 to 1-278, 1-280 to 1-299, 1-301 to 1-307, 1-309, 1-310, 1-321 to 1-325, 1-327, 1-334, 1-335, 1-340 to 1-350, 1-352, 1-353, 1-355, 1-356, 1-358, 1-360, 1-361, 1-362, 1-364 to 1-367, 1-369 to 1-372, 1-375, 1-378 to 1-415, 1-417, 1-418, 1-421 to 1-430, 1-432, 1-434, 1-435, 1-437, 1-439, 1-441 to 1-444, 1-448, 1-450 to 1-453, 1-454 to 1-461, 1-463, 1-465, 1-467 to 1-476, 1-478 to 1-481, 1-485, 1-486, 1-488 to 1-491, 1-493 to 1-496, 1-498 to 1-518, 1-520 to 1-523, 1-525 to 1-527, 1-528, 1-529, 1-531 to 1-536, 1-540, 1-546 to 1-548, 1-551 to 1-555, 1-557, 1-559, 1-560, 1-562 to 1-567, 1-569 to 1-577, 1-579 to 1-588, 1-591 to 1-593, 1-594, 1-595, 1-596, 1-598, 1-599, 1-602, 1-604, 1-606, 1-607, 1-608, 2-002, 3-003, 3-004, 3-005, 3-006, 3-007, 3-008, 3-009, 3-010, 3-011, 3-012, 3-013, 4-001, 4-002, 5-005, 6-001, 6-002, 6-004, 6-005, 6-006, 6-008, 6-009, 6-010, 7-002, 7-003, 7-004, 1-031(S)-(+), 1-039(S)-(−), 1-048(R)-(+), 1-048(S)-(−), 1-050(S)-(+), 1-054(S)-(+), 1-118(+), 1-118(−), B-006, B-008, B-009, B-012, B-014 to B-016, B-018, B-020, B-021, C-001, C-002 and F-001 of the present invention.

### Test Example 2

### Insecticidal Test on Bemisia argentifolii

In styrol cups having an inner diameter of 7 cm, wet filter paper was laid, and kidney bean leaves cut to 3 cm were laid on the paper. 10% emulsifiable concentrates (or 10% wettable powders) of compounds of the present invention were diluted with water containing a spreader to obtain 500 ppm solutions. 2.5 ml of the solutions were sprayed from a rotary spray tower into the styrol cups (2.5 mg/cm²). The leaves were dried in air, and adults of Bemisia argentifolii were released in the cups. The cups were closed and placed in an incubator at 25° C. 5 Days after, dead insects were counted, and the mortality was calculated by using the same equation as in Test Example 1. The test was carried out in duplicate.

Among the compounds tested, the following compounds showed a mortality of at least 90%.

Compounds Nos. 1-011, 1-012, 1-013, 1-018 to 1-023, 1-028, 1-031 to 1-034, 1-037, 1-038 to 1-040, 1-041, 1-042, 1-044, 1-045, 1-046, 1-048 to 1-054, 1-056, 1-059, 1-060, 1-061, 1-063 to 1-070, 1-073, 1-074, 1-076 to 1-090, 1-094 to 1-098, 1-100 to 1-107, 1-109 to 1-113, 1-115 to 1-124, 1-126, 1-127, 1-130, 1-134, 1-138 to 1-144, 1-146, 1-147, 1-149, 1-151, 1-152, 1-153, 1-154, 1-155, 1-157, 1-162, 1-163, 1-165, 1-167, 1-172, 1-173, 1-177, 1-178, 1-179, 1-183, 1-184, 1-188, 1-189, 1-190, 1-193, 1-194, 1-198, 1-201 to 1-206, 1-208, 1-209, 1-210, 1-212, 1-213, 1-220, 1-221, 1-222, 1-223, 1-225 to 1-237, 1-239 to 1-244, 1-248, 1-253, 1-254, 1-255, 1-259 to 1-267, 1-270, 1-271, 1-276, 1-284, 1-288 to 1-291, 1-294 to 1-296, 1-299, 1-303, 1-304, 1-305, 1-308 to 1-311, 1-313 to 1-319, 1-321 to 1-325, 1-327, 1-334, 1-335, 1-356, 1-360, 1-367, 1-369, 1-370, 1-383, 1-384, 1-389, 1-394, 1-396, 1-397, 1-407, 1-408, 1-409, 1-412, 1-414, 1-421, 1-426, 1-428, 1-432, 1-435, 1-438, 1-439, 1-441, 1-443, 1-444, 1-448, 1-458 to 1-461, 1-469, 1-475, 1-478, 1-479, 1-488, 1-490, 1-491, 1-493 to 1-496, 1-499, 1-501, 1-503 to 1-507, 1-509, 1-510, 1-513 to 1-520, 1-522, 1-523, 1-527, 1-528, 1-530, 1-535, 1-536, 1-546, 1-548, 1-551, 1-562 to 1-567, 1-569 to 1-573, 1-575, 1-576, 1-579, 1-581 to 1-584, 1-586 to 1-588, 1-602, 1-606, 3-001, 3-002, 3-004, 3-005, 3-010, 3-012, 3-013, 4-001, 5-004, 6-001, 6-004, 6-008, 6-009, 1-031(S)-(+), 1-039(S)-(−), 1-048(R)-(+), 1-048(S)-(−), 1-050(S)-(+), 1-054(S)-(+), 1-118(+) and 1-118(−) of the present invention.

### Test Example 3

### Insecticidal Test on Myzus persicae

Wet absorbent cotton was laid on glass dishes having an inner diameter of 3 cm, and covered with leaves of Canarium album cut into circles of the same diameter, and 4 apterous adults of Myzus persicae were released. After a day, 10% emulsifiable concentrates (or 10% wettable powders) of compounds of the present invention were diluted with water containing a spreader to obtain 500 ppm solutions, and the solution were sprayed from a rotary spray tower (2.5 mg/cm²). The dishes were covered with lids and placed in an incubator at 25° C. 6 Days after, dead insects were counted, and the mortality was calculated by using the same equation as in Test Example 1. The test was carried out in duplicate.

Among the compounds tested, the following compounds showed a mortality of at least 90%.

Compounds Nos. 1-001, 1-002, 1-003, 1-011, 1-012, 1-013, 1-014, 1-018, 1-019, 1-020, 1-021, 1-022, 1-023, 1-026, 1-027, 1-028, 1-031 to 1-033, 1-035 to 1-068, 1-073 to 1-089, 1-092 to 1-107, 1-109 to 1-129, 1-132, 1-133, 1-134, 1-136, 1-137, 1-139 to 1-144, 1-146 to 1-155, 1-157, 1-158, 1-162 to 1-166, 1-168 to 1-174, 1-177, 1-178, 1-179, 1-183, 1-184, 1-186, 1-188 to 1-196, 1-198 to 1-210, 1-212 to 1-219, 1-220, 1-221, 1-222, 1-223, 1-225 to 1-244, 1-245, 1-247, 1-248, 1-252 to 1-255, 1-259 to 1-325, 1-327 to 1-349, 1-351 to 1-356, 1-360 to 1-365, 1-367 to 1-394, 1-396, 1-397, 1-399 to 1-411, 1-413, 1-414, 1-415, 1-417 to 1-421, 1-423, 1-425, 1-426, 1-427, 1-428, 1-430 to 1-437, 1-439 to 1-444, 1-446 to 1-448, 1-450 to 1-461, 1-463, 1-465, 1-467 to 1-470, 1-473, 1-475, 1-476, 1-477, 1-478 to 1-486, 1-488 to 1-491, 1-493 to 1-501, 1-503 to 1-523, 1-525 to 1-528, 1-529, 1-530 to 1-536, 1-540, 1-546 to 1-548, 1-551 to 1-554, 1-559, 1-560, 1-562 to 1-566, 1-568 to 1-576, 1-579 to 1-589, 1-591 to 1-593, 1-595, 1-596, 1-597, 1-598 to 1-607, 2-002, 3-001 to 3-004, 3-005, 3-006, 3-007, 3-008, 3-009, 3-010, 3-011, 3-012, 3-013, 4-001, 4-002, 5-004, 5-005, 5-007, 6-001, 6-002, 6-004, 6-005, 6-008, 6-009, 7-001 to 7-004, 1-031(S)-(+), 1-039(S)-(−), 1-048(R)-(+), 1-048(S)-(−), 1-050(S)-(+), 1-054(S)-(+), 1-118(+), 1-118(−), A-003, A-004, A-009, B-001 to B-004, B-006, B-008, B-009, B-014 to B-016, B-018, B-020, C-001, C-002, G-002, H-001, 1-001 and J-001 of the present invention.

### Test Example 4

### Insecticidal Test on Aphis gossypii

Wet absorbent cotton was laid on glass dishes having an inner diameter of 3 cm, and covered with leaves of cucumber cut into circles of the same diameter, and 4 apterous adults of Aphis gossypii were released. After a day, 10% emulsifiable concentrates (or 10% wettable powders) of compounds of the present invention were diluted with water containing a spreader to obtain 500 ppm solutions, and the solution were sprayed from a rotary spray tower (2.5 mg/cm²). The dishes were covered with lids and placed in an incubator at 25° C. 6 Days after, dead insects were counted, and the mortality was calculated by using the same equation as in Test Example 1. The test was carried out in duplicate.

Among the compounds tested, the following compounds shoed a mortality of at least 90%.

Compounds Nos. 1-001, 1-002, 1-011, 1-019, 1-020, 1-023, 1-026, 1-028, 1-031, 1-032, 1-033, 1-035 to 1-054, 1-056, 1-057, 1-058, 1-063 to 1-068, 1-073 to 1-080, 1-082 to 1-089, 1-092 to 1-107, 1-109 to 1-118, 1-120 to 1-124, 1-126, 1-134, 1-137, 1-139 to 1-144, 1-146, 1-147, 1-148, 1-149, 1-151, 1-153, 1-154, 1-155, 1-157, 1-158, 1-162, 1-163, 1-165, 1-172, 1-173, 1-175 to 1-178, 1-182, 1-184, 1-186 to 1-194, 1-196, 1-198 to 1-210, 1-212 to 1-223, 1-225 to 1-229, 1-231 to 1-236, 1-238, 1-239, 1-240, 1-242, 1-243, 1-244, 1-246 to 1-249, 1-252, 1-254, 1-255, 1-259 to 1-271, 1-275 to 1-299, 1-301 to 1-323, 1-325, 1-326, 1-327, 1-330 to 1-335, 1-337 to 1-340, 1-343 to 1-356, 1-358, 1-360, 1-362, 1-364 to 1-367, 1-369 to 1-373, 1-375 to 1-387, 1-389, 1-390, 1-392 to 1-397, 1-399 to 1-410, 1-413 to 1-418, 1-420, 1-421, 1-425, 1-426, 1-427, 1-428, 1-429 to 1-435, 1-438 to 1-444, 1-448, 1-450, 1-451, 1-452, 1-454, 1-455, 1-456, 1-457, 1-459, 1-460, 1-461, 1-464, 1-465, 1-469, 1-470, 1-472 to 1-481, 1-484 to 1-486, 1-488 to 1-523, 1-527 to 1-538, 1-541 to 551, 1-554, 1-560, 1-562 to 1-567, 1-569, 1-570, 1-571, 1-573 to 1-577, 1-579 to 1-588, 1-591, 1-593, 1-595, 1-600 to 1-604, 3-001 to 3-013, 4-001, 4-002, 4-003, 5-001, 6-001, 6-002, 6-004, 6-008 to 6-010, 7-002, 7-003, 7-004, 1-031(S)-(+), 1-039(S)-(−), 1-048(R)-(+), 1-048(S)-(−), 1-050(S)-(+), 1-054(S)-(+), 1-118(+), 1-118(−), A-001, A-009, B-001 to B-003, B-006, B-008, B-009, B-014 to B-016 and B-019 to B-022 of the present invention.

### Test Example 5

### Soil Irrigation Test on Myzus persicae

10% emulsifiable concentrates of compounds of the present invention were diluted with tap water to obtain 100 ppm solutions. The soil around the bases of cabbage seedlings (at the 2.5 true leaf stage) planted in plastic cups was irrigated with 10 ml of the solutions, and the cups were placed in a greenhouse. One day after the irrigation, adults of Myzus persicae were released at a ratio of 20 insects per seedling, and the seedlings were incubated in the greenhouse. 6 Days after the release of the insects, living insects were counted, and the control value was calculated from the following equation.

Control value (%)={1−(Cb×Tai)/(Cai×Tb)}×100

Cb: the number of insects in a non-treated area before treatment

Cai: the final number of living insects in a non-treated area

Tb: the number of insects in a treated area before treatment

Tai: the final number of living insects in a treated area

Among the compounds tested, the following compounds showed a control value of at least 90%.

Compounds Nos. 1-011, 1-013, 1-019, 1-023, 1-031 to 1-033, 1-035, 1-036, 1-038, 1-039, 1-041 to 1-045, 1-048, 1-049, 1-050, 1-052, 1-053, 1-054, 1-057, 1-063, 1-073 to 1-076, 1-079, 1-080, 1-084, 1-086, 1-095, 1-096, 1-097, 1-099 to 1-104, 1-106, 1-107, 1-111 to 1-116, 1-118, 1-120, 1-122, 1-124, 1-134, 1-143, 1-146, 1-151, 1-154, 1-155, 1-156, 1-157, 1-158, 1-160, 1-162, 1-163, 1-177, 1-178, 1-179, 1-190, 1-193, 1-194, 1-196, 1-198, 1-201 to 1-210, 1-212, 1-225, 1-226, 1-229 to 1-237, 1-240, 1-242, 1-243, 1-244, 1-246, 1-247, 1-248, 1-249, 1-253, 1-255, 1-259, 1-261 to 1-267, 1-269, 1-270, 1-271, 1-275, 1-276, 1-281, 1-284, 1-285, 1-314, 1-316, 1-318, 1-319, 1-322, 1-323, 1-353, 1-360, 1-369, 1-370, 1-371, 1-383, 1-384, 1-389, 1-390, 1-478, 1-479, 1-484, 1-488 to 1-490, 1-493 to 1-496, 1-498 to 1-501, 1-504 to 1-508, 1-511, 1-513 to 1-518, 1-521, 1-537, 1-538, 1-542, 1-545, 1-547, 1-550, 1-551, 3-004, 3-010, 3-012, 6-001, 6-008, 6-009, 1-031(S)-(+), 1-039(S)-(−), 1-048(R)-(+), 1-048(S)-(−), 1-054(S)-(+), 1-118(+) and 1-118(−) of the present invention.

### Test Example 6

### Test on the Effect of Seed Treatment on Aphis glycines

2.4 mg of compounds of the present invention were diluted with 97.6 μl of acetone. Four soybean seeds were put in each 50 ml plastic tube, and the solutions of compounds of the present invention were poured onto the seeds and stirred until the acetone evaporated completely so that the seeds were evenly coated with the compounds. The treated seeds were sown in pots, 4 seeds per pot, and placed in a greenhouse. After the primary leaf folded out, two adults of Aphis glycines were released per seedling. 7 Days after the release of the insects, living insects were counted, and the control value was calculated from the following equation.

Control value (%)={1−(Cb×Tai)/(Cai×Tb)}×100

wherein

Cb: the number of insects in a non-treated area before treatment

Cai: the final number of living insects in a non-treated area

Tb: the number of insects in a treated area before treatment

Tai: the final number of living insects in a treated area

Among the compounds tested, the following compounds showed a control value of at least 90%.

Compounds Nos. 1-031, 1-033, 1-035, 1-038 to 1-042, 1-048 to 1-054, 1-276, 1-048(R)-(+), 1-048(S)-(−), 1-118(+) and 1-118(−) of the present invention.

### INDUSTRIAL APPLICABILITY

The novel pyrazole derivatives of the present invention are very useful compounds which are excellent in pesticidal activities, especially in insecticidal and miticidal activities, and have little harmful effect on non-target organisms such as mammals, fishes and beneficial insects.

The entire disclosures of Japanese Patent Application No. 2011-025875 filed on Feb. 9, 2011, Japanese Patent Application No. 2011-106993 filed on May 12, 2011, Japanese Patent Application No. 2011-143871 filed on Jun. 29, 2011, Japanese Patent Application No. 2011-176256 filed on Aug. 11, 2011, Japanese Patent Application No. 2011-223837 filed on Oct. 11, 2011, Japanese Patent Application No. 2011-234671 filed on Oct. 26, 2011 and Japanese Patent Application No. 2011-252596 filed on Nov. 18, 2011 including specifications, claims and summaries are incorporated herein by reference in their entireties.

1. A pyrazole derivative of formula (1) or a salt thereof: 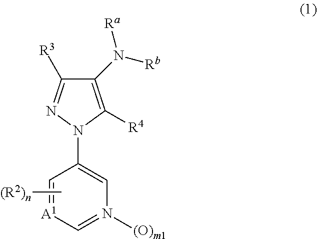
wherein:
A¹ is —N(—O)ₘ₂ or —CR¹;
R¹ and R³ are each independently a hydrogen atom, a halogen atom, cyano, nitro, —OH, —SH, —NH₂, C₁-C₆ alkyl, (C₁-C₆) alkyl optionally substituted with R^{28a}, C₃-C₈ cycloalkyl, (C₃-C₈) cycloalkyl optionally substituted with R^{28a}, C₂-C₆ alkenyl, (C₂-C₆) alkenyl optionally substituted with R^{28a}, C₃-C₈ cycloalkenyl, (C₃-C₈) cycloalkenyl optionally substituted with R^{28a}, C₂-C₆ alkynyl, (C₂-C₆) alkynyl optionally substituted with R^{28a}, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylcarbonyl, C₃-C₈ cycloalkylcarbonyl, C₁-C₆ haloalkylcarbonyl, C₃-C₈ halocycloalkylcarbonyl, C₁-C₆ alkoxycarbonyl, C₁-C₆ haloalkoxycarbonyl, C₁-C₆ alkylaminosulfonyl, di(C₁-C₆ alkyl)aminosulfonyl, C₁-C₆ alkylamino, or di(C₁-C₆ alkyl)amino;
R² is a halogen atom, cyano, nitro, —OH, —SH, —NH₂, C₁-C₆ alkyl, (C₁-C₆) alkyl optionally substituted with R^{28a}, C₃-C₈ cycloalkyl, (C₃-C₈) cycloalkyl optionally substituted with R^{28a}, C₂-C₆ alkenyl, (C₂-C₆) alkenyl optionally substituted with R^{28a}, C₃-C₈ cycloalkenyl, (C₃-C₈) cycloalkenyl optionally substituted with R^{28a}, C₂-C₆ alkynyl, (C₂-C₆) alkynyl optionally substituted with R^{28a}, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylcarbonyl, C₃-C₈ cycloalkylcarbonyl, C₁-C₆ haloalkylcarbonyl, C₃-C₈ halocycloalkylcarbonyl, C₁-C₆ alkoxycarbonyl, C₁-C₆ haloalkoxycarbonyl, C₁-C₆ alkylaminosulfonyl, di(C₁-C₆ alkyl)aminosulfonyl, C₁-C₆ alkylamino, or di(C₁-C₆ alkyl)amino, provided that when n is an integer of at least 2, each R² may be identical with or different from one another;
R⁴ is a hydrogen atom, a halogen atom, cyano, nitro, —OH, —SH, —NH₂, C₁-C₆ alkyl, (C₁-C₆) alkyl optionally substituted with R^{28a}, C₃-C₈ cycloalkyl, (C₃-C₈) cycloalkyl optionally substituted with R^{28a}, C₂-C₆ alkenyl, (C₂-C₆) alkenyl optionally substituted with R^{28a}, C₃-C₈ cycloalkenyl, (C₃-C₈) cycloalkenyl optionally substituted with R^{28a}, C₂-C₆ alkynyl, (C₂-C₆) alkynyl optionally substituted with R^{28a}, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylcarbonyl, C₃-C₈ cycloalkylcarbonyl, C₁-C₆ haloalkylcarbonyl, C₃-C₈ halocycloalkylcarbonyl, C₁-C₆ alkylamino, or di(C₁-C₆ alkyl)amino;
R^{a} is a hydrogen atom, cyano, C₁-C₁₅ alkyl, (C₁-C₁₅) alkyl optionally substituted with R⁵, C₃-C₁₅ cycloalkyl, (C₃-C₁₅) cycloalkyl optionally substituted with R⁵, C₂-C₁₅ alkenyl, (C₂-C₁₅) alkenyl optionally substituted with R⁵, C₃-C₁₂ cycloalkenyl, (C₃-C₁₅) cycloalkenyl optionally substituted with R⁵, C₂-C₁₂ alkynyl, (C₂-C₁₅) alkynyl optionally substituted with R⁵, —OR⁶, —S(O)ᵣR⁶, —C(O)R^{7a}, —C(O)OR^{6a}, —NR^{8c}R^{8d}, —C(═NR^{8b})R^{7a}, —S(O)ᵣN(R^{8a})R⁸, phenyl, phenyl substituted with (Z)_{q}, naphthyl, naphthyl substituted with (Z)_{q}, or D1-1 to D1-99,
R^{b} is —S(O)ᵣR⁶, —C(O)R⁷, —C(S)R⁷, —C(O)OR^{6a}, —C(O)SR^{6a}, —C(S)OR^{6a}, —C(S)SR^{6a}, —C(O)N(Ra)R⁸, —C(S)N(R^{8a})R⁸, —C(O)N(R^{8b})N(R^{8a})R⁸, —C(O)N(R^{8a})OR^{6a}, —C(═NR^{8b})OR^{6a}, C(═NR^{8b})SR^{6a}, —C(═NR^{8b})N(R^{8a})R⁸, —C(═NR^{8b})R⁷, D1-49, D1-51, D1-53, D1-59, D1-61, or D1-63, or R^{b} forms ═C(R^{b2})R^{b3} together with R^{a};
R^{b2} is a hydrogen atom, C₁-C₁₅ alkyl, or —S(O)ᵣR⁶;
R^{b3} is (C₁-C₁₅) alkyl optionally substituted with R¹⁴, —OR⁶, —S(O)ᵣR⁶ or —N(R^{8b})R⁸, or R^{b3} forms, together with R^{b2}, a C₄-C₆ alkylene chain, or a C₄-C₆ alkenylene chain to form a 5 to 7-membered ring together with the carbon atom attached to R^{b3} and R^{b2}, wherein the alkylene chain or the alkenylene chain may contain from 1 to 3 oxygen atoms, sulfur atoms or nitrogen atoms and may optionally be substituted with a halogen atom, a cyano group, a nitro group, C₁-C₆ alkyl, (C₁-C₁₂) alkyl optionally substituted with R¹⁴, —S(O)ᵣR⁶, —C(O)R⁷, —C(S)R⁷, —C(O)OR^{6a}, —C(O)SR^{6a}, —C(S)OR^{6a}, —C(S)SR^{6a}, —C(O)N(R^{8a})R⁸, —C(S)N(R^{8a})R⁸, —C(O)N(R^{8b})N(R^{8a})R⁸, —C(O)N(R^{8a})OR⁶, —C(═NR^{8b})OR^{6a}, —C(═NR^{8b})SR^{6a}, —C(═NR^{8b})N(R^{8a})R⁸, —C(═NR^{8b})R⁷, phenyl, phenyl substituted with (Z)_{q}, an oxo group, a thioxo group, ═NR^{8b} or a C₁-C₆ alkylidene group,
R⁵ is a halogen atom, cyano, nitro, C₃-C₁₅ cycloalkyl, (C₃-C₁₅) cycloalkyl optionally substituted with R¹⁴, —OH, —OR¹¹, —SH, —S(O)ᵣR¹¹, —C(O)R¹², —C(O)OR^{11a}, —C(O)SR^{6a}, —C(S)OR^{6a}, —C(S)SR^{6a}, —C(O)N(R^{13a})R¹³, —C(S)N(R^{13a})R¹³, —C(═NOH)R¹², —C(═NOR¹¹)R¹², —N(R^{13a})R¹³, —Si(R^{9a})(R^{9b})R⁹, —P(O)(OR¹⁰)₂, —P(S)(OR¹⁰)₂, phenyl, phenyl substituted with (Z³)_{q}, naphthyl, naphthyl substituted with (Z)_{q} or D1-1 to D1-99, each of R⁶ and R^{6a} is independently C₁-C₁₅ alkyl, (C₁-C₁₅) alkyl optionally substituted with R¹⁴, C₃-C₁₅ cycloalkyl, (C₃-C₁₅) cycloalkyl optionally substituted with R¹⁴, C₂-C₁₅ alkenyl, (C₂-C₁₅) alkenyl optionally substituted with R¹⁴, C₃-C₁₅ cycloalkenyl, (C₃-C₁₅) cycloalkenyl optionally substituted with R¹⁴, C₂-C₁₅ alkynyl, (C₂-C₁₅) alkynyl optionally substituted with R¹⁴, phenyl, phenyl substituted with (Z)_{q}, naphthyl, naphthyl substituted with (Z)_{q} or D1-1 to D1-99;
R⁷ is a hydrogen atom, C₁-C₁₅ alkyl, (C₁-C₁₅) alkyl optionally substituted with R¹⁴, C₃-C₁₅ cycloalkyl, (C₃-C₁₅) cycloalkyl optionally substituted with R¹⁴, C₂-C₁₅ alkenyl, (C₂-C₁₅) alkenyl optionally substituted with R¹⁴, C₃-C₁₅ cycloalkenyl, (C₃-C₁₅) cycloalkenyl optionally substituted with R¹⁴, C₂-C₁₅ alkynyl, (C₂-C₁₅) alkynyl optionally substituted with R¹⁴, —C(O)R¹², —C(O)OR¹¹, —C(O)N(R^{13a})R¹³, —C(S)N(R^{13a})R¹³, —C(═NOH)R¹², —C(═NOR¹¹)R¹², —C{═NN(R^{13a})R¹³}R¹², phenyl, phenyl substituted with (Z)_{q}, naphthyl, naphthyl substituted with (Z)_{q} or D1-1 to D1-99, or R⁷ forms, together with R^{a}, a C₂-C₆ alkylene chain or a C₂-C₆ alkenylene chain containing a double bond to form a 4 to 8-membered ring together with the carbon atom attached to R⁷ and the nitrogen atom attached to R^{a}, wherein the alkylene chain or the alkenylene chain may contain one or two oxygen atoms, sulfur atoms or nitrogen atoms and may optionally be substituted with a halogen atom, cyano, nitro, C₁-C₆ alkyl, (C₁-C₆) alkyl optionally substituted with R¹⁴, —OH, —OR¹¹, —SH, —S(O)ᵣR¹¹, an oxo group, a thioxo group, ═NR^{8b} or a C₁-C₆ alkylidene group;
R^{7a} is a hydrogen atom or R^{6a};
R⁸ is a hydrogen atom, cyano, R^{6a}, —S(O)ᵣR¹¹, —C(O)R¹², —C(O)OR^{11a}, —C(O)N(R^{13a})R¹³, —C(S)N(R^{13a})R¹³, —C(═NOH)R¹², —C(═NOR¹¹)R¹², —S(O)₂N(R^{13a})R¹³, phenyl, phenyl substituted with (Z)_{q}, naphthyl, naphthyl substituted with (Z)_{q} or D1-1 to D1-99, or R⁸ forms, together with R^{8a}, a C₂-C₇ alkylene chain to form a 3 to 8-membered ring together with the nitrogen atom attached to R⁸ and R^{8a}, wherein the alkylene chain may contain an oxygen atom, a sulfur atom or a nitrogen atom and may optionally be substituted with a halogen atom, cyano, C₁-C₆ alkyl, (C₁-C₆) alkyl optionally substituted with R¹⁴, phenyl, phenyl substituted with (Z)_{q}, an oxo group or a thioxo group, or R⁸ may form ═C(R^{8f})R^{8e} together with R^{8a};
R^{8a} is a hydrogen atom, cyano, C₁-C₁₅ alkyl, (C₁-C₁₅) alkyl optionally substituted with R^{14a}, C₃-C₁₅ cycloalkyl, (C₃-C₁₅) cycloalkyl optionally substituted with R^{14a}, C₂-C₁₅ alkenyl, (C₂-C₁₅) alkenyl optionally substituted with R^{14a}, C₃-C₁₅ cycloalkenyl, (C₃-C₁₅) cycloalkenyl optionally substituted with R^{14a}, C₂-C₁₅ alkynyl or (C₂-C₁₅) alkynyl optionally substituted with R^{14a}, or R^{15a} forms, together with R^{a}, a C₂-C₅ alkylene chain to form a 5 to 8-membered ring together with the nitrogen atom attached to R^{8a} and the nitrogen atom attached to R^{a}, wherein the alkylene chain may contain an oxygen atom, a sulfur atom or a nitrogen atom and may optionally be substituted with a halogen atom, cyano, nitro, C₁-C₆ alkyl, (C₁-C₆) alkyl optionally substituted with R¹⁴, —OH, —OR¹¹, —SH, —S(O)ᵣR¹¹, an oxo group, a thioxo group, or ═NR^{8b};
R^{8b} is a hydrogen atom, cyano, nitro, C₁-C₁₅ alkyl, (C₁-C₁₅) alkyl optionally substituted with R¹⁴, C₃-C₁₅ cycloalkyl, (C₃-C₁₅) cycloalkyl optionally substituted with R¹⁴, C₂-C₁₅ alkenyl, (C₂-C₁₅) alkenyl optionally substituted with R¹⁴, C₃-C₁₅ cycloalkenyl, (C₃-C₁₅) cycloalkenyl optionally substituted with R¹⁴, C₂-C₁₅ alkynyl, (C₂-C₁₅) alkynyl optionally substituted with R¹⁴, —OR¹¹, —S(O)ᵣR¹¹, —C(O)OR^{11a}, —C(O)R¹², —C(O)N(R^{13a})R¹³, —C(S)N(R^{13a})R¹³, or —S(O)₂N(R^{13a})R¹³;
R^{8c} and R^{8d} are each independently a hydrogen atom, C₁-C₆ alkyl, —S(O)ᵣR¹¹, —C(O)OR^{11a}, —C(O)R¹², —C(O)N(R^{13a})R¹³, or —C(S)N(R^{13a})R¹³;
R^{8e} is a hydrogen atom, C₁-C₁₅ alkyl, or —S(O)ᵣR¹¹;
R^{8f} is C₁-C₁₅ alkyl, (C₁-C₁₅) alkyl optionally substituted with R¹⁴, —OR¹¹, —S(O)ᵣR¹¹, —N(R^{13a})R¹³, phenyl, phenyl substituted with (Z)_{q}, naphthyl, naphthyl substituted with (Z)_{q}, or D1-1 to D1-99;
R⁹, R^{9a}, and R^{9b} are each independently C₁-C₆ alkyl;
R¹⁰ is a hydrogen atom or C₁-C₆ alkyl;
R¹¹ and R^{11a} are each independently C₁-C₆ alkyl, (C₁-C₆) alkyl optionally substituted with R^{14a}, C₂-C₆ alkenyl, (C₂-C₆) alkenyl optionally substituted with R^{14a}, C₃-C₈ cycloalkenyl, (C₃-C₈) cycloalkenyl optionally substituted with R^{14a}, C₂-C₆ alkynyl, (C₂-C₆) alkynyl optionally substituted with R^{14a}, C₃-C₈ cycloalkyl, (C₃-C₈) cycloalkyl optionally substituted with R^{14a}, —Si(R^{9a})(R^{9b})R⁹, phenyl, phenyl substituted with (Z)_{q}, naphthyl, naphthyl substituted with (Z)_{q}, or D1-1 to D1-99;
R¹² is a hydrogen atom, R^{11a}, —C(O)R¹⁶, —C(═NOH)R¹⁶, —C(═NOR¹⁵)R¹⁶, phenyl, phenyl substituted with (Z)_{q}, naphthyl, naphthyl substituted with (Z)_{q}, or D1-1 to D1-99;
R¹³ and R^{13a} are each independently a hydrogen atom, R^{11a}, —S(O)ᵣR¹⁵, —C(O)OR^{15a}, —C(O)R¹⁶, —C(O)N(R^{17a})R¹⁷, —C(S)N(R^{17a})R¹⁷, —S(O)₂N(R^{17a})R¹⁷, phenyl, phenyl substituted with (Z)_{q}, naphthyl, naphthyl substituted with (Z)_{q}, or D1-1 to D1-99, or R¹³ and R^{13a} form, together with each other, a C₂-C₇ alkylene chain to form a 3 to 8-membered ring together with the nitrogen atom attached to R¹³ and R^{13a}, wherein the alkylene chain may contain an oxygen atom, a sulfur atom or a nitrogen atom and may optionally be substituted with a halogen atom, cyano, C₁-C₆ alkyl, (C₁-C₆) alkyl optionally substituted with R¹⁴, phenyl, phenyl substituted with (Z)_{q}, an oxo group or a thioxo group;
R¹⁴ and R^{14a} are each independently a halogen atom, cyano, nitro, C₃-C₈ cycloalkyl, (C₃-C₈) cycloalkyl optionally substituted with R¹⁹, —OH, —OR¹⁵, —SH, —S(O)ᵣR¹⁵, —S(═NR^{17b})R^{15a}, —S(O)(═NR^{17b})R^{15a}, —C(O)OH, —C(O)OR^{15a}, —C(O)SR^{15a}, —C(S)OR^{15a}, —C(S)SR^{15a}, —C(O)R¹⁶, —C(O)N(R^{17a})R¹⁷, —C(S)N(R^{17a})R¹⁷, —N(R^{17a})R¹⁷, —C(═NOH)R¹⁶, —C(═NOR¹⁵)R¹⁶, —ON═C(R^{16a})R¹⁶, —S(O)₂N(R^{17a})R¹⁷, —Si(R^{9a})(R^{9b})R⁹, —P(O)(OR¹⁰)₂, —P(S)(OR¹⁰)₂, phenyl, phenyl substituted with (Z)_{q}, naphthyl, naphthyl substituted with (Z)_{q}, or D1-1 to D1-99;
R¹⁵ and R^{15a} are each independently cyano, C₁-C₆ alkyl, (C₁-C₆) alkyl optionally substituted with R¹⁹, C₂-C₆ alkenyl, (C₂-C₆) alkenyl optionally substituted with R¹⁹, C₂-C₆ alkynyl, (C₂-C₆) alkynyl optionally substituted with R¹⁹, C₃-C₈ cycloalkyl, (C₃-C₈) cycloalkyl optionally substituted with R¹⁹, C₃-C₈ cycloalkenyl, (C₃-C₈) cycloalkenyl optionally substituted with R¹⁹, —S(O)ᵣR²⁰, —C(O)OR^{20a}, —C(O)R²¹, —C(O)N(R^{22a})R²², —C(S)N(R^{22a})R²², phenyl, phenyl substituted with (Z)_{q2}, naphthyl, naphthyl substituted with (Z)_{q}, or D1-1 to D1-99;
R¹⁶ and R^{16a} are each independently a hydrogen atom, C₁-C₆ alkyl, (C₁-C₆) alkyl optionally substituted with R¹⁹, C₂-C₆ alkenyl, (C₂-C₆) alkenyl optionally substituted with R¹⁹, C₂-C₆ alkynyl, (C₂-C₆) alkynyl optionally substituted with R¹⁹, C₃-C₈ cycloalkyl, (C₃-C₈) cycloalkyl optionally substituted with R¹⁹, C₃-C₈ cycloalkenyl, (C₃-C₈) cycloalkenyl optionally substituted with R¹⁹, —C(O)R²¹, phenyl, phenyl substituted with (Z)_{q}, naphthyl, naphthyl substituted with (Z)_{q}, or D1-1 to D1-99;
R¹⁷ and R^{17a} are each independently a hydrogen atom, cyano, C₁-C₆ alkyl, (C₁-C₆) alkyl optionally substituted with R¹⁹, C₂-C₆ alkenyl, (C₂-C₆) alkenyl optionally substituted with R¹⁹, C₂-C₆ alkynyl, (C₂-C₆) alkynyl optionally substituted with R¹⁹, C₃-C₈ cycloalkyl, (C₃-C₈) cycloalkyl optionally substituted with R¹⁹, C₃-C₈ cycloalkenyl, (C₃-C₈) cycloalkenyl optionally substituted with R¹⁹, —CHO, —S(O)ᵣR²⁰, —C(O)R²¹, —C(O)OR^{20a}, —C(O)N(R^{22a})R²², —C(S)N(R^{22a})R²² or —S(O)₂N(R^{22a})R²², or R¹⁷ and R^{17a} forms, together with each other, a C₂-C₇ alkylene chain to form a 3 to 8-membered ring together with the nitrogen atom attached to R¹⁷ and R^{17a}, wherein the alkylene chain may contain an oxygen atom, a sulfur atom or a nitrogen atom and may optionally be substituted with a halogen atom, cyano, C₁-C₆ alkyl, (C₁-C₆) alkyl optionally substituted with R¹⁴, phenyl, phenyl substituted with (Z)_{q}, an oxo group or a thioxo group, R^{17b} is a hydrogen atom, cyano, nitro, —OR²⁰, —S(O)ᵣR²⁰, —C(O)OR^{20a}, —C(O)R²¹, —C(O)N(R^{22a})R²², —C(S)N(R^{22a})R²², or —S(O)₂N(R^{22a})R²²;
R¹⁹ is a halogen atom, cyano, C₃-C₈ cycloalkyl, —OR²⁰, —S(O)ᵣR²⁰, —C(O)OR^{20a},
—C(O)N(R^{22a})R²², —C(S)N(R^{22a})R²², —C(═NOR²⁰)R²¹, —Si(R^{9a})(R^{9b})R⁹, phenyl, phenyl substituted with (Z)_{q}, naphthyl, naphthyl substituted with (Z)_{q}, or D1-1 to D1-99;
R²⁰ and R²¹ are each independently a hydrogen atom, C₁-C₆ alkyl, (C₁-C₆) alkyl optionally substituted with R³², C₂-C₆ alkenyl, (C₂-C₆) alkenyl optionally substituted with R³², C₂-C₆ alkynyl, (C₂-C₆) alkynyl optionally substituted with R³², C₃-C₈ cycloalkyl, (C₃-C₈) cycloalkyl optionally substituted with R³², C₃-C₈ cycloalkenyl, (C₃-C₈) cycloalkenyl optionally substituted with R³², phenyl, phenyl substituted with (Z)_{q}, naphthyl, naphthyl substituted with (Z)_{q}, or D1-1 to D1-99;
R^{20a}, R²² and R^{22a} is independently a hydrogen atom, C₁-C₆ alkyl, (C₁-C₆) alkyl optionally substituted with R³², C₂-C₆ alkenyl, (C₂-C₆) alkenyl optionally substituted with R³², C₂-C₆ alkynyl, (C₂-C₆) alkynyl optionally substituted with R³², C₃-C₈ cycloalkyl, (C₃-C₈) cycloalkyl optionally substituted with R³², C₃-C₈ cycloalkenyl, (C₃-C₈) cycloalkenyl optionally substituted with R³², phenyl, phenyl substituted with (Z)_{q}, naphthyl, naphthyl substituted with (Z)_{q}, or D1-1 to D1-99;
D1-1 to D1-99 are rings having the following structures, respectively; 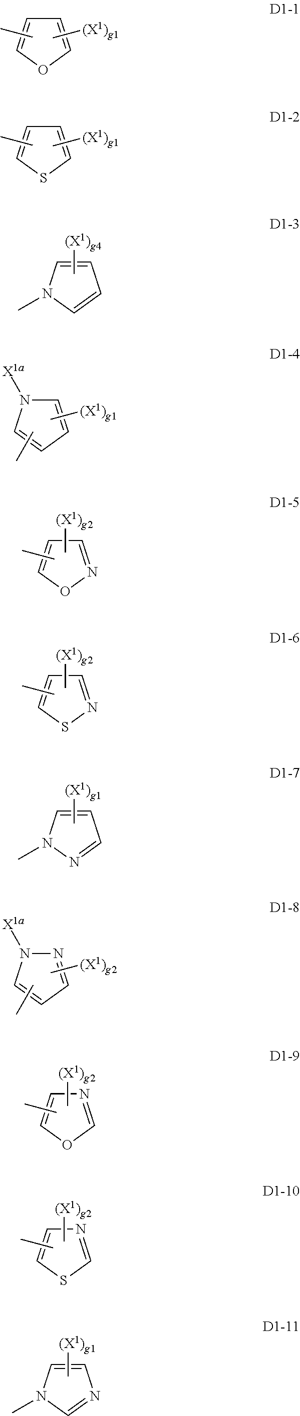 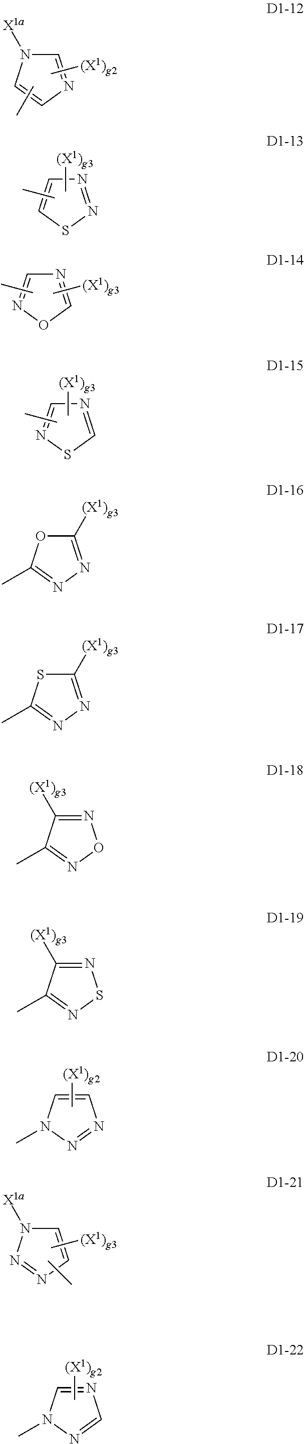 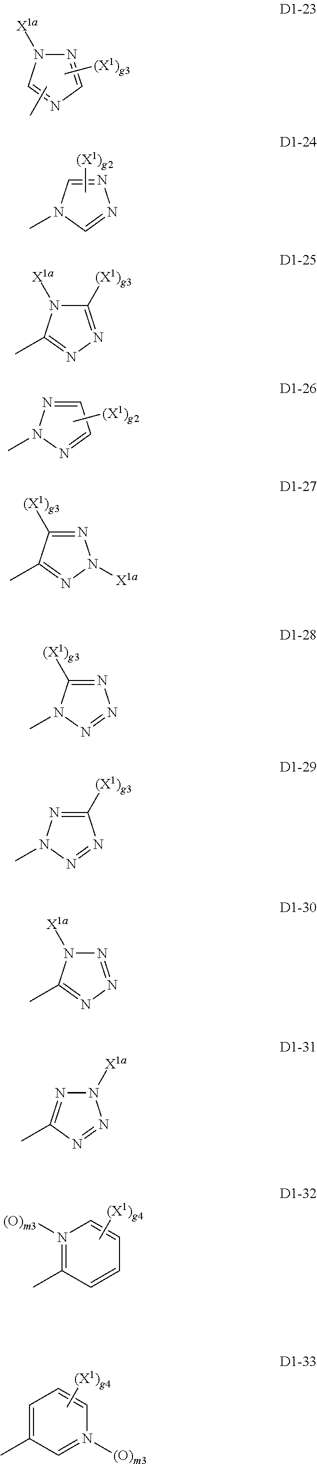 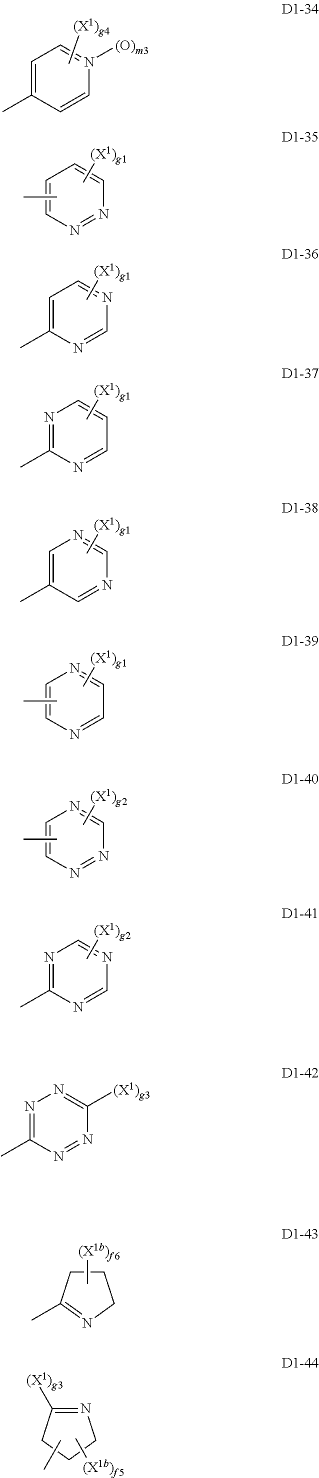 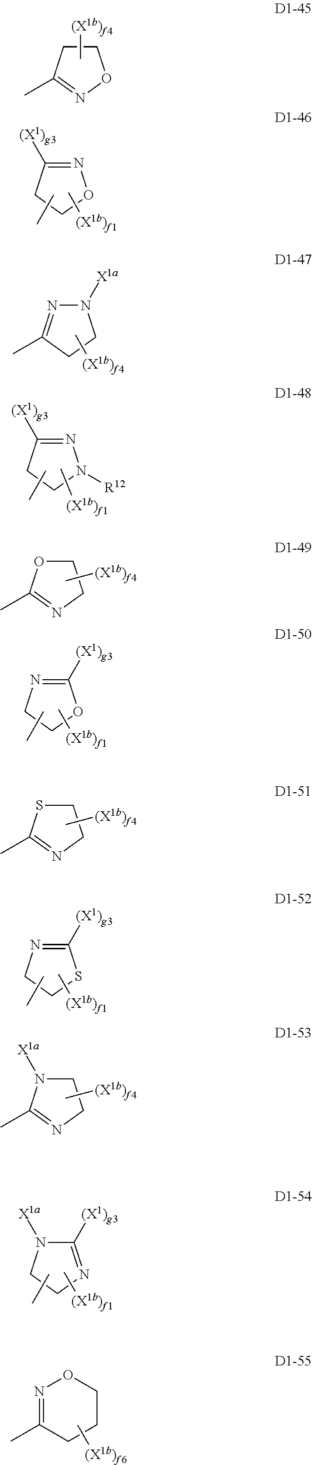 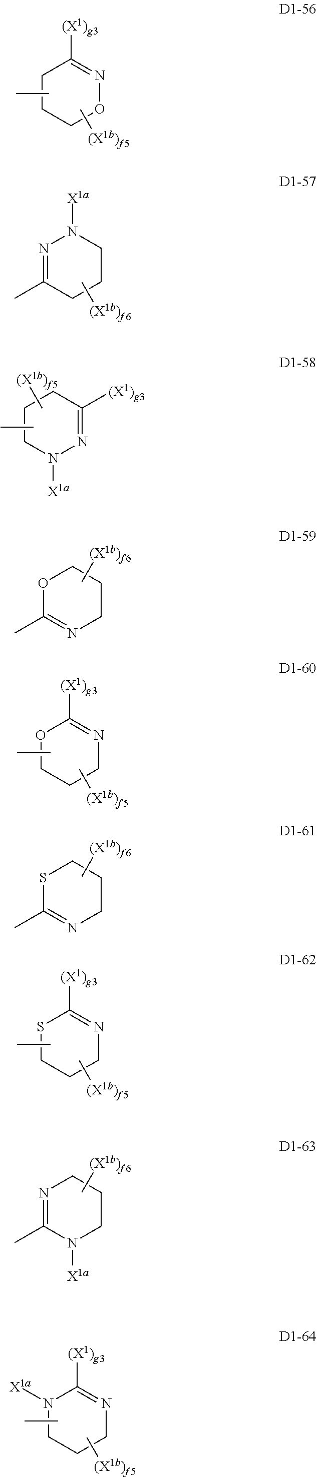 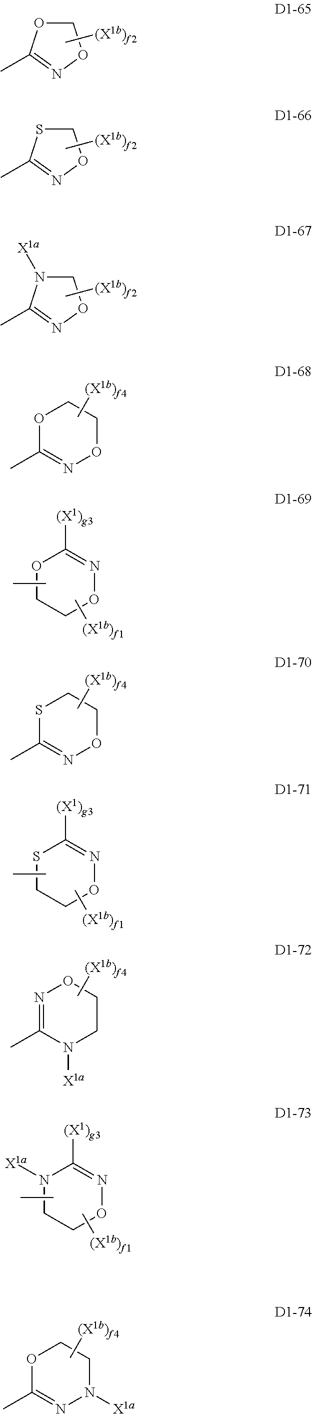 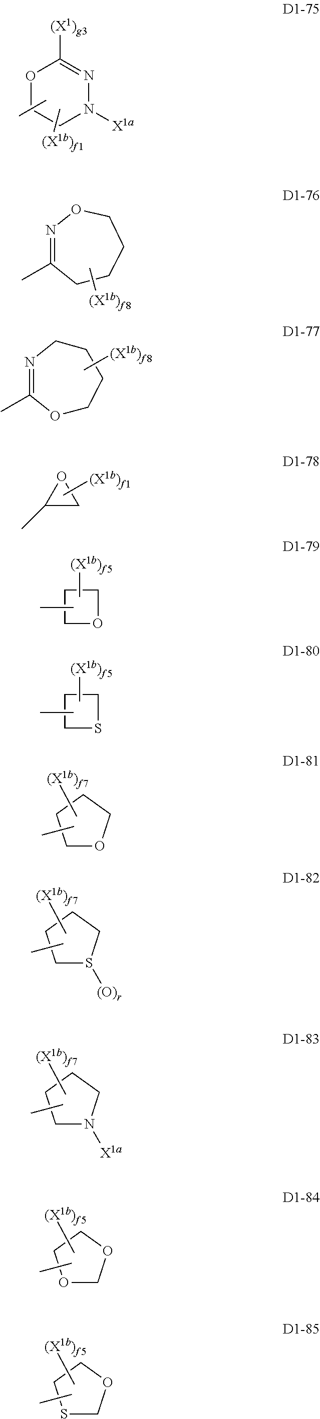 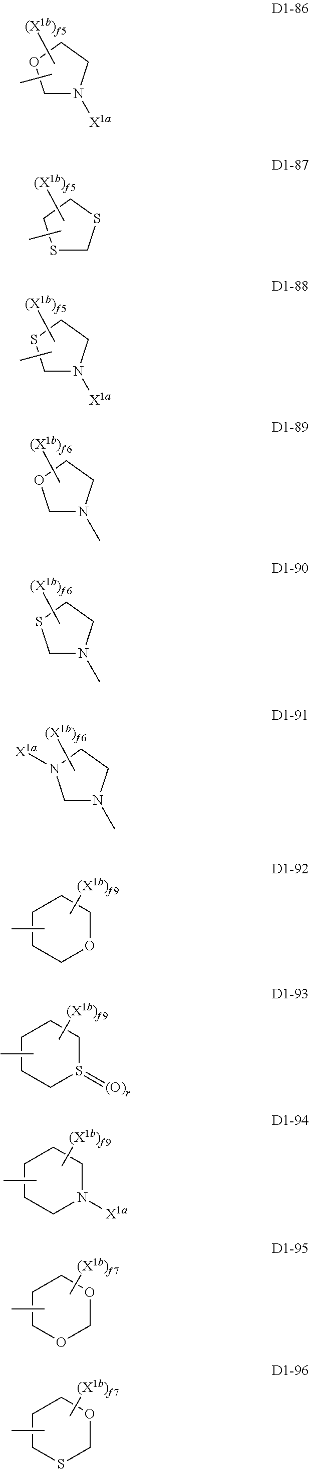 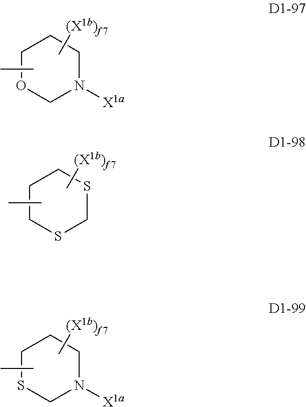
X¹ is a halogen atom, cyano, nitro, —OH, —SH or R²⁴, provided that when g1, g2, or g4 is an integer of at least 2, each X¹ may be identical with or different from one another, and when there are two neighboring X¹'s, the two neighboring X¹'s may form —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —CH₂CH₂CH₂S—, or —OCH₂CH₂S— to form, together with the carbon atoms attached to the X¹'s, a 5-membered ring or 6-membered ring which may have one or more hydrogen atoms on the ring-constituting carbon atoms optionally replaced by one or more halogen atoms, one or more C₁-C₆ alkyl groups, one or more C₁-C₆ haloalkyl groups, one or more C₁-C₆ alkoxy groups, one or more C₁-C₆ alkylthio groups, one or more C₁-C₆ alkylsulfinyl groups, or one or more C₁-C₆ alkylsulfonyl groups;
X^{1a} is a hydrogen atom, cyano, —OH, or R²⁴;
X^{1b} is a halogen atom, cyano, nitro, —OH, —SH, or R²⁴, provided that when f1, f2, f4, f5, f6, f7, f8, or f9 is an integer of at least 2, each X^{1b} may be identical with or different from one another, and when there are two X^{1b}'s on the same carbon, the two X^{1b}'s may form oxo, thioxo, imino, C₁-C₆ alkylimino, C₁-C₆ alkoxyimino, or C₁-C₆ alkylidene together with each other;
Z is a halogen atom, cyano, nitro, —OH, —SH, or R²⁴, provided that q is an integer of at least 2, each Z may be identical with or different from one another, and when there are two neighboring Z's, the two neighboring Z's may form —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —CH₂CH₂CH₂S—, or —OCH₂CH₂S— to form, together with the carbon atoms attached to the Z's, a 5-membered ring or a 6-membered ring which may have one or more hydrogen atoms on the ring-constituting carbon atoms optionally replaced by one or more halogen atoms, one or more C₁-C₆ alkyl groups, one or more C₁-C₆ haloalkyl groups, one or more C₁-C₆ alkoxy groups, one or more C₁-C₆ alkylthio groups, one or more C₁-C₆ alkylsulfinyl groups, or one or more C₁-C₆ alkylsulfonyl groups;
Z³ is a halogen atom, nitro, —OH, —SH, or R²⁴, provided that q is an integer of at least 2, each Z³ may be identical with or different from one another, and when there are two neighboring Z³'s, the neighboring two Z's may form —CH₂CH₂CH₂—, —CH₂CH₂O—, —CH₂OCH₂—, —OCH₂O—, —CH₂CH₂S—, —CH₂SCH₂—, —CH₂CH₂CH₂CH₂—, —CH₂CH₂CH₂O—, —CH₂CH₂OCH₂—, —CH₂OCH₂O—, —OCH₂CH₂O—, —CH₂CH₂CH₂S—, or —OCH₂CH₂S— to form, together with the carbon atoms attached to the Z³'s, a 5-membered ring or a 6-membered ring which may have one or more hydrogen atoms on the ring-constituting carbon atoms optionally replaced by one or more halogen atoms, one or more C₁-C₆ alkyl groups, one or more C₁-C₆ haloalkyl groups, one or more C₁-C₆ alkoxy groups, one or more C₁-C₆ alkylthio groups, one or more C₁-C₆ alkylsulfinyl groups, or one or more C₁-C₆ alkylsulfonyl groups;
R²⁴ is a C₁-C₆ alkyl, (C₁-C₆) alkyl optionally substituted with R²⁸, C₂-C₆ alkenyl, (C₂-C₆) alkenyl optionally substituted with R²⁸, C₃-C₈ cycloalkenyl, (C₃-C₈) cycloalkenyl optionally substituted with R²⁸, C₂-C₆ alkynyl, (C₂-C₆) alkynyl optionally substituted with R²⁸, C₃-C₈ cycloalkyl, (C₃-C₈) cycloalkyl optionally substituted with R²⁸, —OR²⁵, —S(O)ᵣR²⁵, —C(O)OH, —C(O)OR^{25a}, —C(O)R²⁶, —C(O)N(R^{27a})R²⁷, —C(S)N(R^{27a})R²⁷, —N(R^{27a})R²⁷, —C(═NOR²⁵)R²⁶, or —S(O)₂N(R^{27a})R²⁷;
R²⁵, R^{25a}, and R²⁶ are each independently C₁-C₆ alkyl or (C₁-C₆) alkyl optionally substituted with R^{32a};
R²⁷ and R^{27a} are each independently a hydrogen atom, C₁-C₆ alkyl, —S(O)ᵣR³⁴, or —C(O)R³⁴;
R²⁸ and R^{28a} are each independently a halogen atom, —OH, —OR²⁹, —SH, or —S(O)ᵣR²⁹;
R²⁹ and R³³ are each independently C₁-C₆ alkyl;
R³² and R^{32a} are each independently a halogen atom, —OR³³, or —S(O)ᵣR³³;
R³⁴ is C₁-C₆ alkyl, C₃-C₈ cycloalkyl, or C₁-C₆ haloalkyl;
n, g1, and f1 are each independently an integer of from 0 to 3,
g2 and f2 are each independently an integer of from 0 to 2;
g3 is an integer of from 0 to 1;
g4 and f4 are each independently an integer of from 0 to 4;
f5 is an integer of from 0 to 5;
f6 is an integer of from 0 to 6;
f7 is an integer of from 0 to 7;
f8 is an integer of from 0 to 8;
f9 is an integer of from 0 to 9;
q is an integer of from 1 to 5,
q2 is an integer of from 0, 1, 3, 4, or 5;
m1, m2, and m3 are each independently an integer of from 0 or 1; and
r is an integer of from 0 to 2.

2. The pyrazole derivative or a salt thereof according to claim 1, wherein, in formula (1):
R^{b} is —C(O)R⁷, —C(S)R⁷, —C(O)OR^{6a}, —C(O)SR^{6a}, —C(S)OR^{6a}, —C(S)SR^{6a}, —C(O)N(R^{8a})R⁸, —C(S)N(R^{8a})R⁸, —C(O)N(R^{8b})N(R^{8a})R⁸, —C(O)N(R^{8a})OR^{6a}, —C(═NR^{8b})OR^{6a}, —C(═NR^{8b})SR^{6a}, —C(═NR^{8b})N(R^{8a})R⁸, —C(═NR^{8b})R⁷, D1-49, D1-51, D1-53, D1-59, D1-61, or D1-63, or R^{b} forms ═C(R^{b2})R^{b3} together with R^{a}.

3. The pyrazole derivative or a salt thereof according to claim 2, wherein, in formula (1):
A¹ is —CR¹;
R¹ and R³ are each independently a hydrogen atom, a halogen atom, cyano, —NH₂, C₁-C₆ alkyl, (C₁-C₆) alkyl optionally substituted with R^{28a}, C₁-C₆ alkoxy, C₁-C₆ alkylthio, C₁-C₆ alkylsulfinyl, C₁-C₆ alkylsulfonyl, or C₁-C₆ alkoxycarbonyl;
R² is a halogen atom, C₁-C₆ alkyl, or C₁-C₆ alkoxy; and
R⁴ is a hydrogen atom, a halogen atom, cyano, —NH₂, C₁-C₆ alkyl, (C₁-C₆) alkyl optionally substituted with R^{28a}, C₁-C₆ alkoxy, C₁-C₆ alkylthio, C₁-C₆ alkylsulfinyl, or C₁-C₆ alkylsulfonyl.

4. The pyrazole derivative or a salt thereof according to claim 2, wherein, in formula (1);
A¹ is —N(—O)ₘ₂;
R² is a halogen atom, C₁-C₆ alkyl or C₁-C₆ alkoxy; and
R³ and R⁴ are each independently a hydrogen atom, a halogen atom, C₁-C₆ alkyl, or C₁-C₆ alkoxy.

5. The pyrazole derivative or a salt thereof according to claim 3, wherein, in formula (1);
R¹ is a hydrogen atom, a halogen atom, cyano, —NH₂, C₁-C₆ alkyl, C₁-C₆ haloalkyl, or C₁-C₆ alkoxy;
R² is C₁-C₆ alkyl;
R³ is a hydrogen atom, a halogen atom, cyano, C₁-C₆ alkyl, (C₁-C₆) alkyl optionally substituted with R^{28a}, C₁-C₆ alkoxy, C₁-C₆ alkylthio, C₁-C₆ alkylsulfonyl, or C₁-C₆ alkoxycarbonyl;
R⁴ is a hydrogen atom, a halogen atom, C₁-C₆ alkyl, or C₁-C₆ alkylthio;
R^{a} is a hydrogen atom, C₁-C₆ alkyl, (C₁-C₆) alkyl optionally substituted with R⁵, C₂-C₆ alkenyl, C₂-C₆ alkynyl, —S(O)ᵣR⁶, —C(O)R^{7a}, —C(O)OR^{6a}, —NR^{8c}R^{8d} or —C(═NR^{8b})R^{7a},
R^{b} is —C(O)R⁷, —C(S)R⁷, —C(O)OR^{6a}, —C(O)SR^{6a}, —C(O)N(R^{8a})R⁸, —C(S)N(R^{8a})R⁸,
—C(O)N(R^{8b})N(R^{8a})R⁸, —C(═NR^{8b})SR^{6a}, —C(═NR^{8b})N(R^{8a})R⁸, —C(═NR^{8b})R⁷, or D1-51, or R^{b} forms ═C(R^{b2})R^{b3} together with R^{a};
R^{b2} is a hydrogen atom, C₁-C₆ alkyl, or —S(O)ᵣR⁶;
R^{b3} is (C₁-C₆) alkyl optionally substituted with R¹⁴, —OR⁶, or —N(R^{8b})R⁸, or R^{b3} forms, together with R^{b2}, a C₄ alkylene chain to form a 5-membered ring together with the carbon atom attached to R^{b3} and R^{b2}, wherein the alkylene chain may contain a sulfur atom and a nitrogen atom and may optionally be substituted with —C(O)R⁷;
R⁵ is a halogen atom, cyano, C₃-C₈ cycloalkyl, —OR¹¹, —S(O)ᵣR¹¹, —C(O)OR^{11a}, —C(═NOR¹¹)R¹², —Si(R^{9a})(R^{9b})R⁹, phenyl, or D1-37;
R⁶ is C₁-C₆ alkyl or (C₁-C₆) alkyl optionally substituted with R¹⁴;
R^{6a} is C₁-C₆ alkyl or phenyl substituted with (Z)_{q};
R⁷ is C₁-C₁₅ alkyl, (C₁-C₆) alkyl optionally substituted with R¹⁴, C₃-C₈ cycloalkyl, (C₃-C₈) cycloalkyl optionally substituted with R¹⁴, C₂-C₆ alkenyl, (C₂-C₆) alkenyl optionally substituted with R¹⁴, C₃-C₈ cycloalkenyl, C₂-C₆ alkynyl, (C₂-C₆) alkynyl optionally substituted with R¹⁴, —C(O)OR^{11a}, —C(O)R¹², —C(O)N(R^{13a})R¹³, —C(═NOR¹¹)R¹², phenyl substituted with (Z)_{q}, D1-1, D1-2, D1-4, D1-5, D1-6, D1-8, D1-9, D1-10, D1-12, D1-13, D1-19, D1-32, D1-33, D1-35, D1-38, D1-45, D1-81, D1-82, D1-87, D1-88, D1-92, or D1-94, or R⁷ forms, together with R^{a}, a C₂-C₄ alkylene chain or a C₂-C₄ alkenylene chain containing a double bond to form a 4 to 6-membered ring together with the carbon atom attached to R⁷ and the nitrogen atom attached to R^{a}, wherein the alkylene chain may contain one or two oxygen atoms or nitrogen atoms and may optionally be substituted with a halogen atom, C₁-C₆ alkyl, (C₁-C₆) alkyl optionally substituted with R¹⁴, —OR¹¹, —S(O)ᵣR¹¹, an oxo group, or a methylidene group;
R^{7a} is a hydrogen atom or C₁-C₆ alkyl;
R⁸ is a hydrogen atom, C₁-C₆ alkyl, (C₁-C₆) alkyl optionally substituted with R¹⁴, C₃-C₈ cycloalkyl, C₂-C₆ alkenyl, C₂-C₆ alkynyl, —C(O)R¹², —C(O)OR^{11a}, —C(═NOR¹¹)R¹², —S(O)₂R^{11a}, phenyl, phenyl substituted with (Z)_{q}, D1-32, D1-33, D1-34, or D1-80,
R^{8a} is a hydrogen atom, C₁-C₆ alkyl, or (C₁-C₆) alkyl optionally substituted with R^{14a}, or R^{8a} forms, together with R^{a}, a C₂-C₃ alkylene chain to form a 5 to 6-membered ring together with the nitrogen atom attached to R^{8a} and the nitrogen atom attached to R^{a}, wherein the alkylene chain may contain an oxygen atom and may optionally be substituted with (C₁-C₆) alkyl optionally substituted with R¹⁴ or an oxo group, or R⁸ and R^{8a} forms, together with each other, a C₅ alkylene chain to form a 6-membered ring together with the nitrogen atom attached to R⁸ and R^{8a}, wherein the alkylene chain may optionally be substituted with C₁-C₆ alkyl, or R⁸ forms ═C(R^{8f})R^{8e} together with R^{8a};
R^{8b} is a hydrogen atom, cyano, C₁-C₆ alkyl, or —OR¹¹;
R^{8c} is a hydrogen atom, C₁-C₆ alkyl, or —C(O)OR^{11a};
R^{8d} is a hydrogen atom, —C(O)OR^{11a}, or —C(O)R¹²;
R^{8e} is a hydrogen atom;
R^{8f} is —N(R^{13a})R¹³;
R⁹, R^{9a}, R^{9b} and R¹⁰ are each independently C₁-C₆ alkyl;
R¹¹ is C₁-C₆ alkyl, (C₁-C₆) alkyl optionally substituted with R^{14a}, or C₁-C₆ alkenyl;
R^{11a} is C₁-C₆ alkyl, (C₁-C₆) alkyl optionally substituted with R^{14a}, or phenyl;
R¹² is a hydrogen atom, C₁-C₆ alkyl, (C₁-C₆) alkyl optionally substituted with R^{14a}, C₃-C₈ cycloalkyl, —C(═NOR¹⁵)R¹⁶, or D1-2;
R¹³ is C₁-C₆ alkyl or phenyl;
R^{13a} is a hydrogen atom or C₁-C₆ alkyl;
R¹⁴ is a halogen atom, cyano, C₃-C₈ cycloalkyl, —OH, —OR¹⁵, —SH, —S(O)ᵣR¹⁵, —S(═NR^{17b})R^{15a}, —S(O)(═NR^{17b})R^{15a}, —C(O)OH, —C(O)OR^{15a}, —C(O)N(R^{17a})R¹⁷, —C(═NOR¹⁵)R¹⁶, —N(R^{17a})R¹⁷, —ON═C(R^{16a})R¹⁶, —S(O)₂N(R^{17a})R¹⁷, —Si(R^{9a})(R^{9b})R⁹, —P(O)(OR¹⁰)₂, phenyl, phenyl substituted with (Z)_{q}, D1-1, D1-2, D1-5, D1-7, D1-8, D1-28, D1-32, D1-33, D1-34, D1-84, D1-85, D1-87, D1-93 or D1-98;
R^{14a} is a halogen atom, —OR¹⁵, —S(O)ᵣR¹⁵, —Si(R^{9a})(R^{9b})R⁹, or phenyl;
R¹⁵ is cyano, C₁-C₆ alkyl, C₂-C₆ alkenyl, C₂-C₆ alkynyl, (C₁-C₆) alkyl optionally substituted with R¹⁹, —C(O)R²¹, phenyl substituted with (Z)_{q2}, D1-12, D1-32, D1-37, or D1-51;
R^{15a} is C₁-C₆ alkyl;
R¹⁶ is C₁-C₆ alkyl or (C₁-C₆) alkyl optionally substituted with R¹⁹;
R^{16a} is a hydrogen atom
R¹⁷ is a hydrogen atom, C₁-C₆ alkyl, (C₁-C₆) alkyl optionally substituted with R¹⁹, —S(O)ᵣR²⁰, —C(O)OR^{20a}, —C(O)R²¹, —C(O)N(R^{22a})R²², —C(S)N(R^{22a})R²², —C(═NOR²⁰)R²¹, —S(O)₂N(R^{22a})R²², or phenyl;
R^{17a} is a hydrogen atom or C₁-C₆ alkyl, or R^{17a} forms ═C(R^{17c})R^{17d} together with R¹⁷;
R^{17b} is cyano;
R^{17c} is —N(R^{22a})R²²;
R^{17d} is a hydrogen atom;
R¹⁹ is a halogen atom, cyano, C₃-C₈ cycloalkyl, —OR²⁰, —S(O)ᵣR²⁰, —C(O)OR^{20a}, —C(O)N(R^{22a})R²², —C(═NOR²⁰)R²¹, —Si(R^{9a})(R^{9b})R⁹, phenyl, or D1-34;
R²⁰ is C₁-C₆ alkyl or (C₁-C₆) alkyl optionally substituted with R³²;
R^{20a} is C₁-C₆ alkyl;
R²¹ is a hydrogen atom, C₁-C₆ alkyl, or (C₁-C₆) alkyl optionally substituted with R³²;
R²² is C₁-C₆ alkyl;
R^{22a} is a hydrogen atom or C₁-C₆ alkyl;
X¹ is a halogen atom, cyano, nitro, C₁-C₆ alkyl, (C₁-C₆) alkyl optionally substituted with R²⁸, C₃-C₈ cycloalkyl, —OR²⁵, —S(O)ᵣR²⁵, —C(O)R²⁶, —C(═NOR²⁵)R²⁶, or —S(O)₂N(R^{27a})R²⁷, provided that when g2 is an integer of 2, each X¹ may be identical with or different from each other;
X^{1a} is C₁-C₆ alkyl, (C₁-C₆) alkyl optionally substituted with R²⁸, —C(O)OR^{25a}, or —C(O)R²⁶;
X^{1b} is (C₁-C₆) alkyl optionally substituted with R²⁸, provided that when f5 is an integer of 3, each X^{1b} may be identical with or different from one another, and when there are two X^{1b}'s on the same carbon, the two X^{1b}'s may form oxo together with each other;
Z is a halogen atom, nitro, C₁-C₆ alkyl, (C₁-C₆) alkyl optionally substituted with R²⁸, —S(O)ᵣR²⁵, —C(O)OH, or —C(O)N(R^{27a})R²⁷, provided that when q is an integer of at least 2, and there are two neighboring Z's, the two neighboring Z's may form —OCH₂O— to form, together with the carbon atoms attached to the Z's, a 5-membered ring which may have one or more hydrogen atoms on the ring-constituting carbon atoms optionally replaced by one or more halogen atoms;
R²⁵ and R²⁶ are each independently C₁-C₆ alkyl or (C₁-C₆) alkyl optionally substituted with R^{32a};
R^{25a}, R²⁹, R³³, and R³⁴ is independently C₁-C₆ alkyl;
R²⁷ is a hydrogen atom or —S(O)ᵣR³⁴;
R^{27a} is a hydrogen atom;
R²⁸ and R^{28a} are each independently a halogen atom, —OH, —OR²⁹, or —S(O)ᵣR²⁹;
R³² is a halogen atom, —OR³³, or —S(O)ᵣR³³;
R^{32a} is a halogen atom or —S(O)ᵣR³³;
n is an integer of from 0 to 1;
g1 and g2 are each independently an integer of from 0 to 2;
g3, f7, f9, and m3 are each independently an integer of 0;
g4 is an integer of from 0 to 1;
f5 is an integer of from 0 to 3;
q is an integer of from 1 to 2;
q2 is an integer of 1; and
r is an integer of from 0 to 2.

6. The pyrazole derivative or a salt thereof according to claim 1, wherein, in formula (1):
R¹, R², and R⁴ are hydrogen atoms;
R^{a} is a hydrogen atom or C₁-C₆ alkyl;
R^{b} is —S(O)ᵣR⁶;
R⁶ is C₁-C₆ alkyl, C₂-C₆ alkenyl, or (C₁-C₆) alkyl optionally substituted with R¹⁴;
R¹⁴ is —S(O)ᵣR¹⁵;
R¹⁵ is C₁-C₆ alkyl; and
r is an integer of from 0 to 2.

7. The pyrazole derivative or a salt thereof according to claim 5, wherein, in formula (1):
R⁷ is C₁-C₁₅ alkyl, (C₁-C₆) alkyl optionally substituted with R¹⁴, C₃-C₈ cycloalkyl, (C₃-C₈) cycloalkyl optionally substituted with R¹⁴, C₂-C₆ alkenyl, (C₂-C₆) alkenyl optionally substituted with R¹⁴, C₃-C₈ cycloalkenyl, C₂-C₆ alkynyl, (C₂-C₆) alkynyl optionally substituted with R¹⁴, —C(O)OR^{11a}, —C(O)R¹², —C(O)N(R^{13a})R¹³, —C(═NOR¹¹)R¹², phenyl substituted with (Z)_{q}, D1-1, D1-2, D1-4, D1-5, D1-6, D1-8, D1-9, D1-10, D1-12, D1-13, D1-19, D1-32, D1-33, D1-35, D1-38, D1-45, D1-81, D1-82, D1-87, D1-88, D1-92 or D1-94;
R^{8a} is a hydrogen atom, C₁-C₆ alkyl or (C₁-C₆) alkyl optionally substituted with R^{14a}, or R⁸ forms, together with R^{8a}, a C₅ alkylene chain to form a 6-membered ring together with the nitrogen atom attached to R⁸ and R^{8a}, wherein the alkylene chain may optionally be substituted with C₁-C₆ alkyl, or R⁸ and R^{8a} forms ═C(R^{8f})R^{8e} together with each other.

8. The pyrazole derivative or a salt thereof according to claim 5, wherein, in formula (1):
R⁷ forms, together with R^{a}, a C₂-C₄ alkylene chain or a C₂-C₄ alkenylene chain containing a double bond to form a 4 to 6-membered ring together with the carbon atom attached to R⁷ and the nitrogen atom attached to R^{a}, wherein the alkylene chain may contain one or two oxygen atoms or nitrogen atoms and may optionally be substituted with a halogen atom, C₁-C₆ alkyl, (C₁-C₆) alkyl optionally substituted with R¹⁴, —OR¹¹, —S(O)ᵣR¹¹, an oxo group, or a methylidene group;
R⁸ is a hydrogen atom, C₁-C₆ alkyl, (C₁-C₆) alkyl optionally substituted with R¹⁴, C₃-C₈ cycloalkyl, C₂-C₆ alkenyl, C₂-C₆ alkynyl, —C(O)R¹², —C(O)OR^{11a}, —C(═NOR¹¹)R¹², —S(O)₂R^{11a}, phenyl, phenyl substituted with (Z)_{q}, D1-32, D1-33, D1-34 or D1-80;
R^{8a} forms, together with R^{a}, a C₂-C₃ alkylene chain to form a 5 to 6-membered ring together with the nitrogen atom attached to R^{8a} and the nitrogen atom attached to R^{a}, wherein the alkylene chain may contain an oxygen atom and may optionally be substituted with (C₁-C₆) alkyl optionally substituted with R¹⁴ or an oxo group;
R¹⁴ is a halogen atom or —S(O)ᵣR¹⁵; and
R¹⁵ is C₁-C₆ alkyl.

9. The pyrazole derivative or a salt thereof according to claim 4, wherein, in formula (1):
R⁴ is a hydrogen atom;
R³ is a halogen atom;
R^{a} is C₁-C₆ alkyl;
R^{b} is —C(O)R⁷;
R⁷ is (C₁-C₆) alkyl optionally substituted with R¹⁴;
R¹⁴ is —S(O)ᵣR¹⁵;
R¹⁵ is C₁-C₆ alkyl; and
m2, n, and r are integers of 0.

10. A pesticide comprising an active ingredient comprising a pyrazole derivative or a salt thereof according to claim 1.

11. An agrochemical comprising an active ingredient comprising a pyrazole derivative or a salt thereof according to claim 1.

12. A parasticide for an internal or external parasite in or on a mammal or bird, which comprises an active ingredient comprising a pyrazole derivative or a salt thereof according to claim 1.

13. An insecticide or miticide comprising an active ingredient comprising a pyrazole derivative or a salt thereof according to claim 1.

14. A seed treatment agent comprising an active ingredient comprising a pyrazole derivative or a salt thereof according to claim 1.

15. The seed treatment agent according to claim 14, which treats seeds by dipping.

16. A soil treatment agent comprising an active ingredient comprising a pyrazole derivative or a salt thereof according to claim 1.

17. The soil treatment agent according to claim 16, which treats soil by irrigation.
